(19) United States
(12) Patent Application Publication
(10) Pub. No.: US 2026/0167656 A1
(43) Pub. Date: Jun. 18, 2026

(54) PHOSPHORAMIDATE COMPOUNDS AND USES THEREOF

(71) Applicant: ALLORION THERAPEUTICS INC, Natick, MA (US)

(72) Inventors: Tiansheng WANG, Concord, MA (US); Qiang DING, Guangzhou (CN); Fang LI, Lexington, MA (US)

(21) Appl. No.: 19/119,356

(22) PCT Filed: Oct. 7, 2023
(86) PCT No.: PCT/CN2023/123253

§ 371 Date: Apr. 8, 2025

(30) Foreign Application Priority Data
Oct. 9, 2022 (WO) PCT/CN2022/124090

(51) Int. Cl. C07F 9/6558; A61K 31/675; A61P 35/00; C07B 59/00; C07F 9/564; C07F 9/6561

(52) U.S. Cl. CPC C07F 9/65583; A61K 31/675; A61P 35/00; C07B 59/004; C07F 9/564; C07F 9/65586; C07F 9/6561; C07B 2200/05

(57) ABSTRACT

Provided herein are novel compounds (e.g., Formula I, II, or III), pharmaceutical compositions, and methods of using such as for treating cancer.

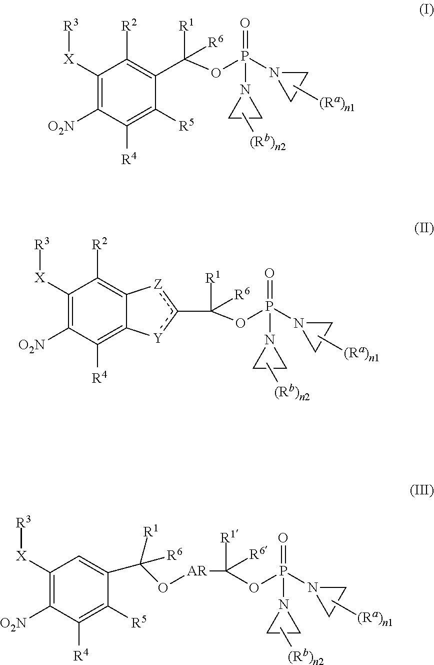

### CROSS-REFERENCE TO RELATED APPLICATIONS

This application claims priority to International Patent Application No. PCT/CN2022/124090, filed on Oct. 9, 2022, the entirety of which is incorporated herein by reference.

### FIELD

In various embodiments, the present disclosure generally relates to novel compounds, compositions comprising the same, methods of preparing and methods of using the same, e.g., for treating or preventing various diseases or disorders described herein.

### BACKGROUND

Aldo-keto reductase family 1 member C3 (AKR1C3) is a member of the aldo-keto reductase (AKR) superfamily, which catalyzes the conversion of aldehydes and ketones to their corresponding alcohols by using NADH and/or NADPH as cofactors. AKR1C3 was known to be overexpressed in various cancers such as prostate cancer, non-small cell lung cancer, etc. AKR1C3 is also a biomarker of NRF2 activation. Aberrant activation of NRF2 pathway, such as those caused by gain of function genetic alterations in NRF2 or loss of function genetic alterations in KEAP1 or CUL3, can lead to elevated expression of its target genes, including AKR1C3. Aberrant activation of NRF2 pathway has been implicated in various cancers and associated with poor prognosis. Novel compounds that can selectively target or utilize aberrant activation of NRF2 pathway and/or AKR1C3 are needed.

### BRIEF SUMMARY

In various embodiments, the present disclosure is based in part on the discovery of novel compounds that can selectively inhibit the growth of cancer cells with abnormal AKR1C3 activities and/or overexpression of AKR1C3. As detailed in the Examples section herein, the tested exemplified compounds herein typically can have a lower IC₅₀ value in inhibiting cancer cell growth in the absence of an AKR1C3 inhibitor compared to those observed in the presence of an AKR1C3 inhibitor. Thus, it is expected that the novel compounds herein can selectively target those cancers characterized as having abnormal AKR1C3 activity and/or an overexpressed level of AKR1C3, for example, as a result of aberrant activation of NRF2 pathway, and can have a better safety profile.

Some embodiments of the present disclosure are directed to a compound of Formula I, II, or III, or a pharmaceutically acceptable salt thereof,

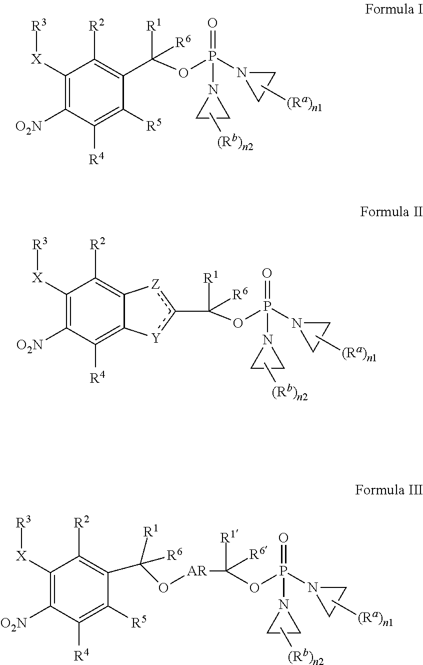

wherein the variables are defined herein. In some embodiments, the compound of Formula I can be characterized as having a structure according to any of the subformulae described herein such as: Formula I-1, I-2, 1-3, I-4, I-5, I-6, I-7, 1-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8 as defined herein. In some embodiments, the compound of Formula II can be characterized as having a structure according to any of the subformulae described herein such as: II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1 as defined herein. In some embodiments, the compound of Formula III can be characterized as having a structure according to any of the subformulae described herein such as: III-1, III-2, III-3, or III-4. In some embodiments, the present disclosure provides a compound according to any of those described in Tables A1 to A18, or a pharmaceutically acceptable salt thereof. In some embodiments, the present disclosure provides a compound according to Examples 1-569, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure provides a method of treating or preventing cancer, such as those having abnormal AKR1C3 activity and/or overexpression of AKRIC3, in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., I-1, 1-2, 1-3, 1-4, 1-5, I-6, 1-7, 1-8, 1-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof, or a pharmaceutical composition comprising the same.

In some embodiments, the present disclosure also provides a method of treating or preventing cancer in a subject in need thereof, which comprises administering to the subject an effective amount of a compound of the present disclosure (e.g., a compound of Formula I (e.g., I-1, 1-2, 1-3, I-4, 1-5, I-6, I-7, 1-8, 1-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof) or an effective amount of a pharmaceutical composition described herein. In some embodiments, the cancer is characterized as having an abnormal AKR1C3 activity and/or overexpression of AKR1C3. In some embodiments, the cancer is characterized as having an NRF2/KEAP1 pathway mutation which causes an aberrant NRF2 activation.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intravenous injection.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as additional anticancer agents described herein.

It is to be understood that both the foregoing summary and the following detailed description are exemplary and explanatory only, and are not restrictive of the invention herein.

### DETAILED DESCRIPTION

In various embodiments, the present disclosure provides compounds and compositions that are useful for treating or preventing various diseases or disorders described herein, e.g., cancer.

### Compounds

Typically, the compounds disclosed herein are substrates of AKRIC3, which can be activated by AKR1C3, for example, to release a phosphoramidate.

### Formula I

In some embodiments, the present disclosure provides a compound of Formula I, or a pharmaceutically acceptable salt thereof:

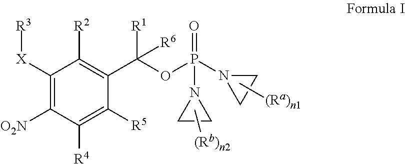

wherein:
(1) R¹ is hydrogen (in additional embodiment, deuterium), optionally substituted C₁₋₄ alkyl, optionally substituted C₂₋₄ alkenyl, or optionally substituted C₂₋₄ alkynyl; and
   R², R⁴, and R⁵ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring; or
(2) R¹ and R², together with the intervening atoms, are joined to form an optionally substituted 4-8 membered carbocyclic or heterocyclic ring, and
   R⁴ and R⁵ are as defined in (1); or
(3) R¹ and R⁵, together with the intervening atoms, are joined to form an optionally substituted 4-8 membered carbocyclic or heterocyclic ring; and
   R² and R⁴ are as defined in (1); or
(4) R⁴ and R⁵, together with the intervening atoms, are joined to form an optionally substituted 4-8 membered ring; and
   R¹ and R² are as defined in (1) or (2); and
wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
R³ is hydrogen, an optionally substituted C₁₋₄ alkyl, or an optionally substituted 3-10 membered ring;
R⁶ is hydrogen (in additional embodiment, deuterium), optionally substituted C₁₋₄ alkyl, optionally substituted C₂₋₄ alkenyl, or optionally substituted C₂₋₄ alkynyl; the integers n1 and n2 are each independently 0, 1, 2, 3, or 4;
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atom(s), are joined together to form an optionally substituted 3-6 membered ring, and any remaining instances of R^{a} and/or R^{b} are as defined above.

In some embodiments, the compound of Formula I (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula I can exist in the form of an individual enantiomer and/or diastereomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as an individual enantiomer substantially free (e.g., with less than 20%, less than 10%, less than 5%, less than 1%, by weight, by HPLC or SFC area, or both, or with a non-detectable amount) of the other enantiomer, for example, the compound can have an enantiomeric excess (“ee”) of greater than 60%, such as 80% ee or greater, 90% ee or greater, 95% ee or greater, 98% ee or greater, 99% ee or greater, etc. In some embodiments, when applicable, the compound of Formula I (including any of the applicable sub-formulae as described herein) can also exist as a mixture of stereoisomers in any ratio, such as a racemic mixture.

In some embodiments, the compound of Formula I (including any of the applicable sub-formulae as described herein) can exist as an isotopically labeled compound, particularly, a deuterated analog, wherein one or more of the hydrogen atoms of the compound of Formula I is/are substituted with a deuterium atom with an abundance above its natural abundance, e.g., a CD₃ analog when the compound has a CH₃ group.

It should be apparent to those skilled in the art that in certain cases, the compound of Formula I may exist as a mixture of tautomers. The present disclosure is not limited to any specific tautomer. Rather, the present disclosure encompasses any and all of such tautomers whether or not explicitly drawn or referred to.

As shown herein, compounds with various cyclizations among R¹, R², R⁴, and R⁵ in accordance with Formula I can be activated by AKR1C3 to release the phosphoramidate moiety in Formula I.

For example, in some embodiments according to Formula I, R¹ and R², together with the intervening atoms, are joined to form an optionally substituted 4-8 membered carbocyclic or heterocyclic ring, wherein R⁴ and R⁵ are defined herein, for example, in some embodiments, both R⁴ and R⁵ are hydrogen. As used herein, the “4-8 membered carbocyclic or heterocyclic ring” refers to any ring system that is not fully aromatic and has 4-8 ring members, and in the case of a heterocyclic ring, contains one or more ring heteroatoms. It should be noted that when substituted, the 4-8 membered carbocyclic or heterocyclic ring can be substituted at any available position(s), and two or more substituents can also be optionally joined with the intervening atom(s) to form additional fused, bridged, or spiro ring structure(s), unless the substituent(s) is specified to be otherwise. Carbocyclic or heterocyclic rings that have different designations of ring members should be understood similarly.

In some embodiments according to Formula I, R¹ and R², together with the intervening atoms, are joined to form an optionally substituted 5-7 membered carbocyclic ring. Typically, when substituted, the 5-7 membered carbocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring. In one embodiment, two substituents are optionally joined with the intervening atom(s) to form an optionally substituted 3-6 membered ring. In one embodiment, a “C₁₋₄ heteroalkyl having one or two heteroatoms” has one oxygen atom, one nitrogen atom, one oxygen and one nitrogen atom, two oxygen atoms, or two nitrogen atoms. Non-limiting examples of “C₁₋₄ heteroalkyl having one or two heteroatoms” include C₁₋₄ alkoxy, NH(C₁₋₄ alkyl), N(C₁₋₃ alkyl)(C₁₋₃ alkyl), provided that the total number of carbon is less than 4; —CH₂—O—C₁₋₃ alkyl; —CH₂—OH; —CH₂—NH₂, —CH₂—NH(C₁₋₃ alkyl); —CH₂—N(CH₃)₂; etc. As used herein, a “3-6 membered ring” refers to any ring having 3-6 ring members, which can be carbocyclic, heterocyclic, aryl, or heteroaryl, wherein a ring carbon atom may exist as C═O, such as in a pyrrolidinone ring, ring nitrogen and sulfur atoms can be optionally oxidized. Non-limiting examples of “3-6 membered ring” include cyclopropyl, cyclobutyl, azetidinyl, oxetanyl, 5-membered heteroaryl, phenyl, 6-membered heteroaryl, 5 or 6 membered heterocyclyl, etc. When the “3-6 membered ring” is said to be substituted, the substituent(s) can be attached at any available position(s), and two or more substituents can also be optionally joined with the intervening atom(s) to form additional fused, bridged, or spiro ring structure(s), unless the substituent(s) is specified to be otherwise. Rings that have different designations of ring members herein should be understood similarly.

In some specific embodiments according to Formula I, the compound can be characterized as having a structure according to Formula I-1, I-2, or I-3:

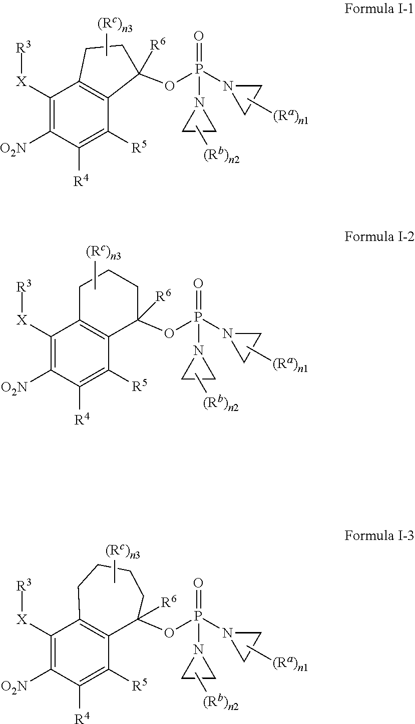

wherein:
the integer n3 is 0, 1, or 2; and
R^{c} at each occurrence is independently oxo, F, OH, NH₂, C₁₋₄ alkyl optionally substituted with F, or C₁₋₄ heteroalkyl having one or two heteroatoms and optionally substituted with F; or two instances of R^{c} are joined with the intervening atom(s) to form an optionally substituted 3-6 membered ring; and
the other variables are defined herein.

In some embodiments, n3 is 0. For example, in some embodiments, the compound of Formula I-1 can be characterized as having a structure according to Formula I-1-A or I-1-B:

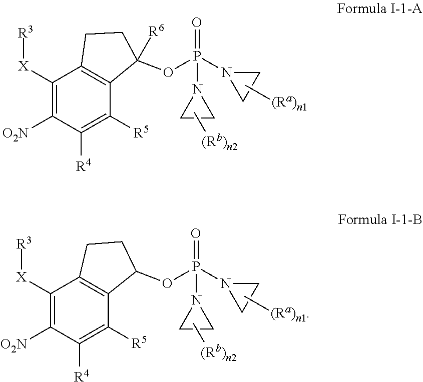

As shown herein, one of the enantiomers in accordance with Formula I-1 may have a more potent antiproliferation activity. In some embodiments, the compound of Formula I-1 can be an individual isomer having 80% ee or higher, such as having 90% ee or higher, 95% ee or higher, 98% ee or higher, or 99% ee or higher, or the other enantiomer is not detectable by HPLC or SFC. For example, in some embodiments, the compound of Formula I-1-B can be a substantially pure enantiomer according to Formula I-1-B-E1:

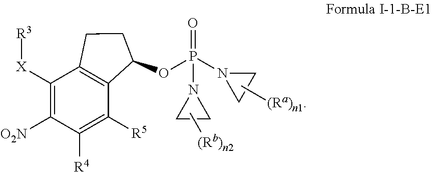

A “substantially pure enantiomer” as used herein refers to such enantiomer that has an 80% ee or higher, such as having 90% ee or higher, 95% ee or higher, 98% ee or higher, or 99% ee or higher, or the other enantiomer is not detectable by HPLC or SFC.

In some embodiments, the compound of Formula I-1-B can be a substantially pure enantiomer according to Formula I-1-B-E2:

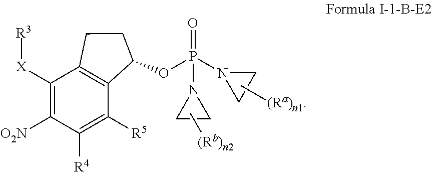

In some embodiments, n3 in Formula I-1, I-2, or I-3 can also be 1 or 2. For example, in some embodiments, n3 is 1 or 2, and R^{c} at each occurrence is independently oxo, F, OH, NH₂, C₁₋₄ alkyl optionally substituted with F, or C₁₋₄ heteroalkyl having one or two heteroatoms and optionally substituted with F. In one embodiment, two instances of R^{c} are joined with the intervening atom(s) to form an optionally substituted 3-6 membered ring. In one embodiment, two germinal R^{c} are joined with the intervening atom(s) to form an optionally substituted 3-6 membered ring, which forms a spiro ring system with the ring formed by R¹ and R². In one embodiment, two adjacent R^{c} are joined with the intervening atom(s) to form an optionally substituted 3-6 membered ring, which forms a fused ring system with the ring formed by R′ and R². In one embodiment, two non-germinal and non-adjacent R^{c} are joined with the intervening atom(s) to form an optionally substituted 3-6 membered ring, which forms a bridged ring system with the ring formed by R¹ and R².

In some embodiments according to Formula I, R¹ and R², together with the intervening atoms, are joined to form an optionally substituted 5-7 membered heterocyclic ring having one or two ring heteroatoms independently selected from O, N, and S. In some embodiments, the 5-7 membered heterocyclic ring has one ring heteroatom, which can be oxygen or nitrogen, particularly, oxygen. Typically, when substituted, the 5-7 membered heterocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring. In one embodiment, two substituents are optionally joined with the intervening atom(s) to form an optionally substituted 3-6 membered ring.

In some specific embodiments, the compound of Formula I can be characterized as having a structure according to Formula I-4, I-5, or I-6:

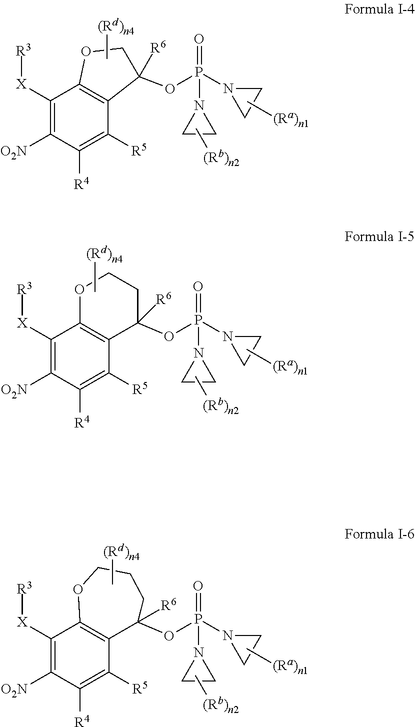

wherein:
the integer n4 is 0, 1, or 2; and
R^{d} at each occurrence is independently oxo, F, OH, NH₂, C₁₋₄ alkyl optionally substituted with F, or C₁₋₄ heteroalkyl having one or two heteroatoms and optionally substituted with F; or two instances of R^{d} are joined with the intervening atom(s) to form an optionally substituted 3-6 membered ring; and
the other variables are defined herein.

In some embodiments, n4 is 0. For example, in some embodiments, the compound of Formula I-5 can be characterized as having a structure according to Formula I-5-A or I-5-B:

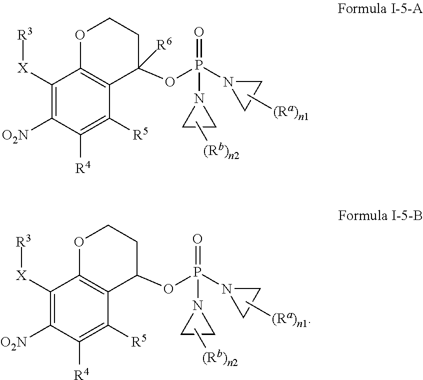

In some embodiments, the compound of Formula I-5 can be an individual isomer having 80% ee or higher, such as having 90% ee or higher, 95% ee or higher, 98% ee or higher, or 99% ee or higher, or the other enantiomer is not detectable by HPLC or SFC. For example, in some embodiments, the compound of Formula I-5-B can be a substantially pure enantiomer according to Formula I-5-B-E1:

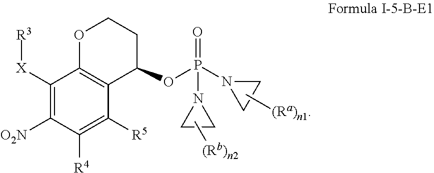

In some embodiments, the compound of Formula I-5-B can be a substantially pure enantiomer according to Formula I-5-B-E2:

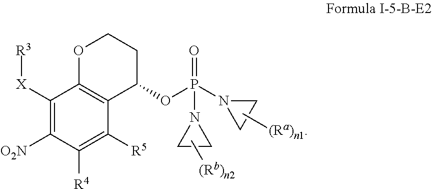

In some embodiments, n4 in Formula I-4, I-5, or I-6 can also be 1 or 2. For example, in some embodiments, n4 is 1 or 2, and R^{d} at each occurrence is independently oxo, F, OH, NH₂, C₁₋₄ alkyl optionally substituted with F, or C₁₋₄ heteroalkyl having one or two heteroatoms and optionally substituted with F.

In some embodiments, in Formula I, R¹ and R⁵, together with the intervening atoms, are joined to form an optionally substituted 4-8 membered carbocyclic or heterocyclic ring, whereas R² and R⁴ are defined herein, for example, both R² and R⁴ can be hydrogen.

In some embodiments according to Formula I, R¹ and R⁵, together with the intervening atoms, are joined to form an optionally substituted 5-7 membered carbocyclic ring. Typically, when substituted, the 5-7 membered carbocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring. In one embodiment, two substituents are optionally joined with the intervening atom(s) to form an optionally substituted 3-6 membered ring.

In some specific embodiments according to Formula I, the compound can be characterized as having a structure according to Formula I-7, I-8, or I-9:

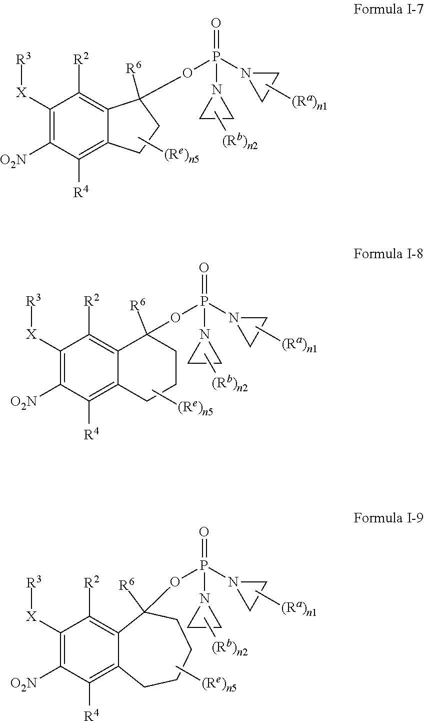

wherein:
the integer n5 is 0, 1, or 2; and
R^{e} at each occurrence is independently oxo, F, OH, NH₂, C₁₋₄ alkyl optionally substituted with F, or C₁₋₄ heteroalkyl having one or two heteroatoms and optionally substituted with F; or two instances of R^{e} are joined with the intervening atom(s) to form an optionally substituted 3-6 membered ring; and
the other variables are defined herein.

In some embodiments, n5 is 0. For example, in some embodiments, the compound of Formula I-7 can be characterized as having a structure according to Formula I-7-A or I-7-B:

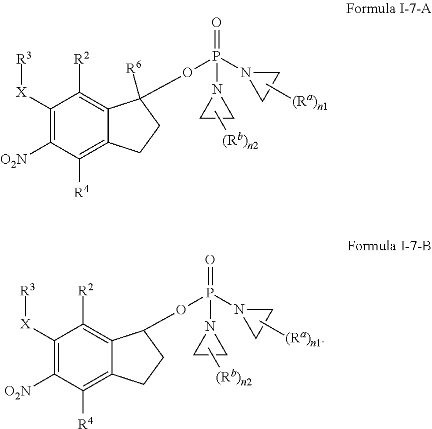

In some embodiments, the compound of Formula I-7 can be an individual isomer having 80% ee or higher, such as having 90% ee or higher, 95% ee or higher, 98% ee or higher, or 99% ee or higher, or the other enantiomer is not detectable by HPLC or SFC. For example, in some embodiments, the compound of Formula I-7-B can be a substantially pure enantiomer according to Formula I-7-B-E1:

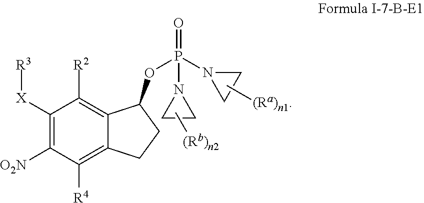

In some embodiments, the compound of Formula I-7-B can be a substantially pure enantiomer according to Formula I-7-B-E2:

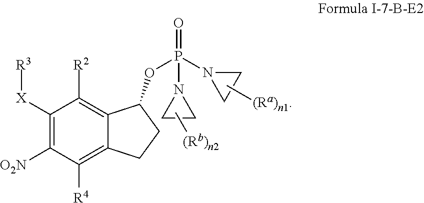

In some embodiments, n5 in Formula I-7, I-8, or I-9 can also be 1 or 2. For example, in some embodiments, n5 is 1 or 2, and R^{e} at each occurrence is independently oxo, F, OH, NH₂, C₁₋₄ alkyl optionally substituted with F, or C₁₋₄ heteroalkyl having one or two heteroatoms and optionally substituted with F.

In some embodiments according to Formula I, R¹ and R⁵, together with the intervening atoms, are joined to form an optionally substituted 5-7 membered heterocyclic ring having one or two ring heteroatoms independently selected from O, N, and S. In some embodiments, the 5-7 membered heterocyclic ring has one ring heteroatom, which can be oxygen or nitrogen, particularly, oxygen. Typically, when substituted, the 5-7 membered heterocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring. In one embodiment, two substituents are optionally joined with the intervening atom(s) to form an optionally substituted 3-6 membered ring.

In some specific embodiments, the compound of Formula I can be characterized as having a structure according to Formula I-10, I-11, or I-12:

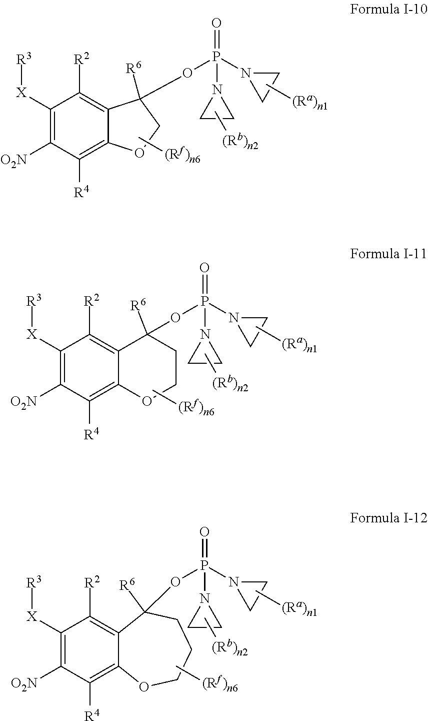

wherein:
the integer n6 is 0, 1, or 2; and
R^{f} at each occurrence is independently oxo, F, OH, NH₂, C₁₋₄ alkyl optionally substituted with F, or C₁₋₄ heteroalkyl having one or two heteroatoms and optionally substituted with F; or two instances of R^{f} are joined with the intervening atom(s) to form an optionally substituted 3-6 membered ring; and
the other variables are defined herein.

In some embodiments, n6 is 0. For example, in some embodiments, the compound of Formula I-11 can be characterized as having a structure according to Formula I-11-A or I-11-B:

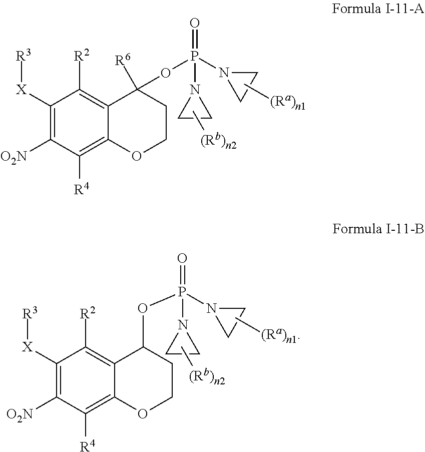

In some embodiments, the compound of Formula I-11 can be an individual isomer having 80% ee or higher, such as having 90% ee or higher, 95% ee or higher, 98% ee or higher, or 99% ee or higher, or the other enantiomer is not detectable by HPLC or SFC. For example, in some embodiments, the compound of Formula I-11-B can be a substantially pure enantiomer according to Formula I-11-B-E1:

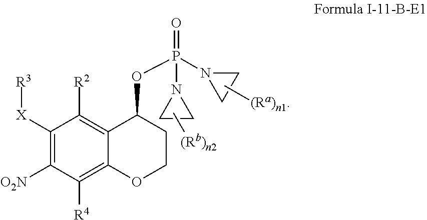

In some embodiments, the compound of Formula I-11-B can be a substantially pure enantiomer according to Formula I-11-B-E2:

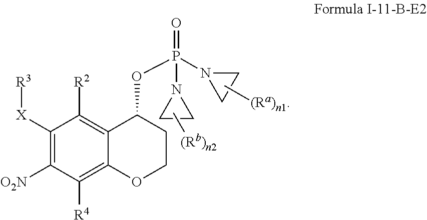

In some embodiments, n6 in Formula I-10, I-11, or I-12 can also be 1 or 2. For example, in some embodiments, n6 is 1 or 2, and R^{f} at each occurrence is independently oxo, F, OH, NH₂, C₁₋₄ alkyl optionally substituted with F, or C₁₋₄ heteroalkyl having one or two heteroatoms and optionally substituted with F.

In some embodiments according to Formula I, R⁴ and R⁵, together with the intervening atoms, can be joined to form an optionally substituted 4-8 membered ring.

In some embodiments according to Formula I, R¹, R², R⁴, and R⁵ do not form any ring structure among each other. In such embodiments, R¹ is hydrogen, optionally substituted C₁₋₄ alkyl, optionally substituted C₂₋₄ alkenyl, or optionally substituted C₂₋₄ alkynyl; and R², R⁴, and R⁵ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring. For example, in some embodiments, R¹ can be hydrogen or C₁₋₄ alkyl optionally substituted with F, such as methyl, CF₃, etc.; and each of R², R⁴, and R⁵ can be hydrogen. In another embodiment, R¹ is deuterium.

Typically, the integers n1 and n2 in Formula I are each independently 0, 1, or 2. In some embodiments, at least one of n1 and n2 is not 0.

In some embodiments, the integers n1 and n2 in Formula I are both 0 or both 1. For example, in some embodiments, the compound of Formula I (e.g., Formula I-1, I-1-A, I-1-B, I-1-B-E1, I-1-B-E2, I-2, I-3, I-4, I-5, I-6, I-5-A, I-5-B, I-5-B-E1, I-5-B-E2, I-7, 1-8, I-9, I-7-A, I-7-B, I-7-B-E1, I-7-B-E2, I-10, I-11, I-12, I-11A, I-11-B, I-11-B-E1, I-11-B-E2, etc.) is characterized as having both n1 and n2 as 0.

In some embodiments, the compound of Formula I (e.g., Formula I-1, I-1-A, I-1-B, I-1-B-E1, I-1-B-E2, I-2, I-3, I-4, I-5, I-6, I-5-A, I-5-B, I-5-B-E1, I-5-B-E2, I-7, I-8, I-9, I-7-A, I-7-B, I-7-B-E1, I-7-B-E2, I-10, I-11, I-12, I-11A, I-11-B, I-11-B-E1, I-11-B-E2, etc.) is characterized as having both n1 and n2 as 1. For example, in some embodiments, the compound of Formula I can be characterized as having a structure according to Formula I-13:

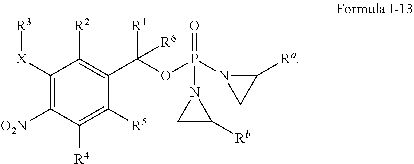

When n1 and n2 are both 1, R^{a} and R^{b} in Formula I (e.g., Formula I-1, I-1-A, I-1-B, I-1-B-E1, I-1-B-E2, I-2, I-3, I-4, I-5, I-6, I-5-A, I-5-B, I-5-B-E1, I-5-B-E2, I-7, I-8, I-9, I-7-A, I-7-B, I-7-B-E1, I-7-B-E2, I-10, I-11, I-12, I-11A, I-11-B, I-11-B-E1, I-11-B-E2, I-13, etc.) can be the same or different as defined herein. In some embodiments, n1 and n2 are both 1, R^{a} and R^{b} in Formula I are the same, which can be a C₁₋₄ alkyl, such as methyl, ethyl, or isopropyl.

In some embodiments, n1 and n2 are both 1, R^{a} and R^{b} in Formula I are both methyl. For example, in some embodiments, the compound of Formula I-13 can be characterized as having a structure according to Formula I-13-A or I-13-B:

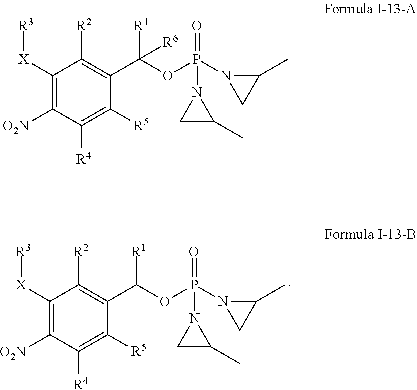

In some embodiments, the compound of Formula I-13 can be an individual isomer having 80% ee or higher, such as having 90% ee or higher, 95% ee or higher, 98% ee or higher, or 99% ee or higher, or the other enantiomer is not detectable by HPLC or SFC. For example, in some embodiments, the compound of Formula I-13-B can be a substantially pure enantiomer according to Formula I-13-B-E1:

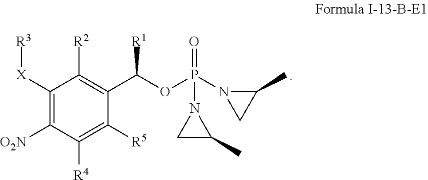

In some embodiments, the compound of Formula I-13-B can be a substantially pure enantiomer according to Formula I-13-B-E2:

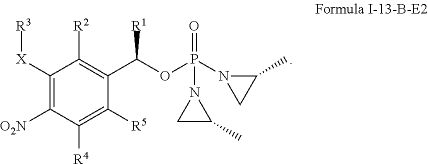

In some embodiments, the compound of Formula I-13-B can be a substantially pure enantiomer according to Formula I-13-B-E3:

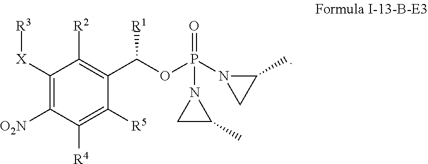

In some embodiments, the compound of Formula I-13-B can be a substantially pure enantiomer according to Formula I-13-B-E4:

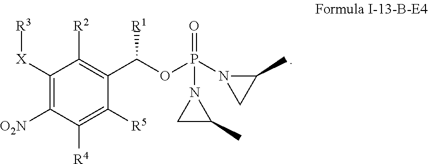

Various groups are suitable for R⁶ in Formula I. However, typically, R⁶ in Formula I (e.g., any of the applicable subformulae herein) is hydrogen. In one embodiment, R⁶ is deuterium. In one embodiment, R⁶ is optionally substituted C₁₋₄ alkyl. In one embodiment, R⁶ is CH₃. In one embodiment, R⁶ is CF₃.

Various groups are suitable for R⁴ in Formula I. Typically, R⁴ in Formula I (e.g., any of the applicable subformulae herein) is hydrogen.

When R² is not forming a ring with R¹, R² in Formula I (e.g., any of the applicable subformulae herein) is also typically hydrogen. Other definitions of R² are described herein.

When R⁵ is not forming a ring with R¹ or R⁴, R⁵ in Formula I (e.g., any of the applicable subformulae herein) is also typically hydrogen. Other definitions of R⁵ are described herein.

X in Formula I (e.g., any of the applicable subformulae herein) is typically O. In one embodiment, X is S. In one embodiment, X is NR¹⁰. In one embodiment, X is NH.

In some embodiments, X in Formula I (e.g., any of the applicable subformulae herein) can be a C₁₋₄ alkylene, such as CH₂.

In some embodiments, X in Formula I (e.g., any of the applicable subformulae herein) can be a C₁₋₄ heteroalkylene, such as those having one or two heteroatoms independently O, S, and N. For example, in some embodiments, X in Formula I (e.g., any of the applicable subformulae herein) can be —O—CH₂—. In one embodiment, X is —CH₂—O—. Unless otherwise specified, the left point of attachment of X is to R³.

R³ in Formula I (e.g., any of the applicable subformulae herein) is typically an optionally substituted 3-10 membered ring. For example, in some embodiments, R³ is an optionally substituted phenyl, an optionally substituted 5 or 6-membered heteroaryl, or an optionally substituted bicyclic heteroaryl (e.g., 8-10 membered bicyclic heteroaryl).

In some embodiments, R³ in Formula I (e.g., any of the applicable subformulae herein) is phenyl.

In some embodiments, R³ in Formula I (e.g., any of the applicable subformulae herein) is a phenyl, which is substituted with 1-3 substituents each independently selected from halogen, CN, OH, NH₂, COOH, CONH₂, G¹, OG¹, NHG¹, NG¹G¹, C(O)G¹, COOG¹, CONHG¹, CONG¹G¹, OC(O)G¹, OCOOG¹, OCONHG¹, OCONG¹G¹, NHG¹C(O)G¹, NHG¹COOG¹, NHG¹CONHG¹, NHG¹CONG¹G¹, NG¹G¹C(O)G¹, NG¹G¹COOG¹, NG¹G¹CONHG¹, NG¹G¹CONG¹G¹, SO₂G¹, SO₂NHG¹, or SO₂NG¹G¹, wherein G¹ at each occurrence is independently an optionally substituted C₁₋₄ alkyl, an optionally substituted C₂₋₄ alkenyl, an optionally substituted C₂₋₄ alkynyl, or an optionally substituted 3-6 membered ring structure, such as cyclopropyl, cyclobutyl, phenyl, pyridyl, etc., or two G¹ of an NG¹G¹ can join together with the nitrogen atom to form an optionally substituted 4-8 membered heterocyclic ring. In some embodiments, R³ in Formula I (e.g., any of the applicable subformulae herein) is a phenyl, which is substituted with 1-3 substituents each independently selected from halogen, CN, OH, NH₂, COOH, CONH₂, G¹, OG¹, SG₁, NHG¹, NG¹G¹, C(O)G¹, COOG¹, CONHG¹, CONG¹G¹, OC(O)G¹, OCOOG¹, OCONHG¹, OCONG¹G¹, NHC(O)G¹, NHCOOG¹, NHCONHG¹, NHCONG¹G¹, NG¹C(O)G¹, NG¹COOG¹, NG¹CONHG¹, NG¹CONG¹G¹, SO₂G¹, SO₂NHG¹, or SO₂NG¹G¹, wherein G¹ at each occurrence is independently an optionally substituted C₁₋₄ alkyl, an optionally substituted C₂₋₄ alkenyl, an optionally substituted C₂₋₄ alkynyl, or an optionally substituted 3-6 membered ring structure, such as cyclopropyl, cyclobutyl, phenyl, pyridyl, etc., or two G¹ of an NG¹G¹ can join together with the nitrogen atom to form an optionally substituted 4-8 membered heterocyclic ring.

In some embodiments, R³ in Formula I (e.g., any of the applicable subformulae herein) is a phenyl, which is substituted with 1-3 substituents each independently selected from F, Cl, CN, OH, NH₂, COOH, CONH₂, G², OG², NHG², NG²G², C(O)G², COOG², CONHG², CONG²G², SO₂G², SO₂NHG², or SO₂NG²G², wherein G² at each occurrence is independently a C₁₋₄ alkyl, C₃₋₆ cycloalkyl, 3-6 membered heterocyclic ring having 1-2 ring heteroatoms, phenyl, or 5 or 6-membered heteroaryl, each of which is optionally substituted with 1-3 substituents independently selected from F, Cl, OH, NH₂, C₁₋₄ alkyl optionally substituted with 1-3 F, or C₁₋₄ heteroalkyl having 1 or 2 heteroatoms and optionally substituted with 1-3 F, or two G² of an NG²G² can join together with the nitrogen atom to form an optionally substituted 4-8 membered heterocyclic ring having 0 or 1 additional ring heteroatom.

In some embodiments, R³ in Formula I (e.g., any of the applicable subformulae herein) is a phenyl, which is substituted with 1-3 (such as 1 or 2) substituents each independently selected from
(i) Halogen or CN, particularly, F or Cl,
(ii) C₁₋₄ alkyl optionally substituted with 1-3 F, such as CF₃,
(iii) C₁₋₄ alkoxy optionally substituted with 1-3 F, such as OCF₃,
(iv) S—C₁₋₄ alkyl optionally substituted with 1-3 F, such as —S—CH₃,
(v) SO₂—C₁₋₄ alkyl optionally substituted with 1-3 F, such as —SO₂—CH₃,
(vi) 3-4 membered ring, such as cyclopropyl or cyclobutyl,
(vii) O-(3-4 membered ring), such as O-cyclopropyl, O-cyclobutyl, or O-oxetanyl, etc.
(viii) CONH(C₁₋₄ alkyl), or CON(C₁₋₄ alkyl)(C₁₋₄ alkyl), such as CONHCH₃ or CON(CH₃)₂,
(ix) CONH(3-4 membered ring), such as CONH(cyclopropyl) or CONH(cyclobutyl),
(x) SO₂NH(C₁₋₄ alkyl), or SO₂N(C₁₋₄ alkyl)(C₁₋₄ alkyl), such as SO₂NHCH₃ or SO₂N(CH₃)₂,
(xi) SO₂NH(3-4 membered ring),
(xii) CO-(4-7 membered heterocyclyl), e.g., the 4-7 membered heterocyclyl is azetidine, pyrrolidine, piperazine, piperidine, etc., which is optionally substituted with 1-3 substituents independently F or methyl;
(xiii) CO—NH(5 or 6 membered heteroaryl), e.g., CO—NH-pyridyl,
(xiv) phenyl optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.
(xv) 6-membered heteroaryl such as pyridine, pyrimidine, pyridone, or pyrimidinone, etc., which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.
(xvi) 5-membered heteroaryl such as pyrazine, oxadiazole, etc., which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.; and
(xvii) 5-7 membered heterocyclic ring having 1-2 ring heteroatoms, such as 5- or 6-membered lactam ring, which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.

In some embodiments, R³ in Formula I (e.g., any of the applicable subformulae herein) can also be a 6-membered heteroaryl such as pyridyl

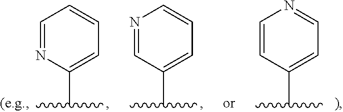

which is optionally substituted with 1-3 (such as 1 or 2) substituents each independently selected from
(i) Halogen or CN, particularly, F or Cl,
(ii) C₁₋₄ alkyl optionally substituted with 1-3 F, such as CF₃,
(iii) C₁₋₄ alkoxy optionally substituted with 1-3 F, such as OCF₃,
(iv) S—C₁₋₄ alkyl optionally substituted with 1-3 F, such as —S—CH₃,
(v) SO₂—C₁₋₄ alkyl optionally substituted with 1-3 F, such as —SO₂—CH₃,
(vi) 3-4 membered ring, such as cyclopropyl or cyclobutyl,
(vii) O-(3-4 membered ring), such as O-cyclopropyl, O-cyclobutyl, or O-oxetanyl, etc.
(viii) CONH(C₁₋₄ alkyl), or CON(C₁₋₄ alkyl)(C₁₋₄ alkyl), such as CONHCH₃ or CON(CH₃)₂,
(ix) CONH(3-4 membered ring), such as CONH(cyclopropyl) or CONH(cyclobutyl),
(x) SO₂NH(C₁₋₄ alkyl), or SO₂N(C₁₋₄ alkyl)(C₁₋₄ alkyl), such as SO₂NHCH₃ or SO₂N(CH₃)₂,
(xi) SO₂NH (3-4 membered ring),
(xii) CO-(4-7 membered heterocyclyl), e.g., the 4-7 membered heterocyclyl is azetidine, pyrrolidine, piperazine, piperidine, etc., which is optionally substituted with 1-3 substituents independently F or methyl;
(xiii) CO—NH(5 or 6 membered heteroaryl), e.g., CO—NH-pyridyl,
(xiv) phenyl optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.
(xv) 6-membered heteroaryl such as pyridine, pyrimidine, pyridone, or pyrimidinone, etc., which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.
(xvi) 5-membered heteroaryl such as pyrazine, oxadiazole, etc., which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.; and
(xvii) 5-7 membered heterocyclic ring having 1-2 ring heteroatoms, such as 5- or 6-membered lactam ring, which is optionally substituted with 1 or 2 substituents independently selected from halogen (e.g., F), CN, C₁₋₄ alkyl optionally substituted with 1-3 F, C₁₋₄ alkoxy optionally substituted with 1-3 F, and a 3-4 membered ring such as cyclopropyl, cyclobutyl, etc.

In some embodiments, R³ in Formula I (e.g., any of the applicable subformulae herein) can be a phenyl or pyridyl which is substituted with 1-3 substituents each independently selected from the following: (1) F, Cl, Br, or CN, (2) C₁₋₄ alkyl optionally substituted with 1-3 F, such as methyl, ethyl, CF₃, (3) C₁₋₄ alkoxy optionally substituted with 1-3 F, such as methoxy, ethoxy, isopropoxy, OCF₃, OCH₂CF₃, etc., (4) C₁₋₄ alkythio optionally substituted with 1-3 F, such as CH₃S—, (5) C₁₋₄ alkylsulfone optionally substituted with 1-3 F, such as CH₃SO₂—, (6) a 3-6 membered ring, such as cyclopropyl, cyclobutyl, or oxetanyl, (7) (3-6 membered ring)-O, such as cyclopropoxy, cyclobutoxy,

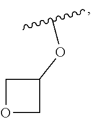 [SMILES: *OC1COC1]

etc., (8) SO₂N(C₁₋₄ alkyl)(C₁₋₄ alkyl), such as SO₂N(CH₃)₂, etc.

In some embodiments, R³ in Formula I (e.g., any of the applicable subformulae herein) can be a monosubstituted phenyl or pyridyl, such as

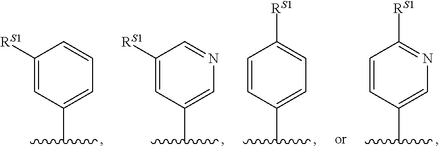

wherein R^{S1} is selected from: (1) F, Cl, Br, or CN, (2) C₁₋₄ alkyl optionally substituted with 1-3 F, such as methyl, ethyl, CF₃, (3) C₁₋₄ alkoxy optionally substituted with 1-3 F, such as methoxy, ethoxy, isopropoxy, OCF₃, OCH₂CF₃, etc., (4) C₁₋₄ alkythio optionally substituted with 1-3 F, such as CH₃S—, (5) C₁₋₄ alkylsulfone optionally substituted with 1-3 F, such as CH₃SO₂—, (6) a 3-4 membered ring, such as cyclopropyl, cyclobutyl, or oxetanyl, (7) (3-4 membered ring)-O, such as cyclopropoxy, cyclobutoxy,

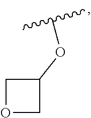 [SMILES: *OC1COC1]

etc., (8) SO₂N(C₁₋₄ alkyl)(C₁₋₄ alkyl), such as SO₂N(CH₃)₂, etc.

In some more specific embodiments, R³ in Formula I (e.g., any of the applicable subformulae herein) can be selected from:

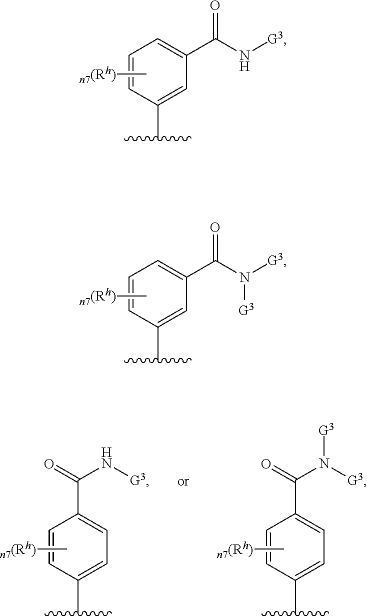

wherein:
G³ at each occurrence is independently an optionally substituted C₁₋₄ alkyl or optionally substituted 3-6 membered ring, such as cyclopropyl, cyclobutyl, oxetanyl

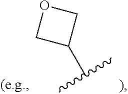 [SMILES: *C1COC1]

etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-7 membered (in additional embodiment, 4-8 membered) heterocyclic ring having 0 or 1 additional ring heteroatom, wherein the 4-7 membered (in additional embodiment, 4-8 membered) heterocyclic ring is optionally substituted with 1-3 substituents each independently oxo, F, C₁₋₄ alkyl, OH, NH₂, or C₁₋₄ heteroalkyl having 1-2 heteroatoms; and wherein:
the integer n7 is 0, 1, or 2; and
R^{h} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

In one embodiment, the compound is a compound having a structure according to Formula IV-1, IV-7, or IV-8, or a pharmaceutically acceptable salt thereof:

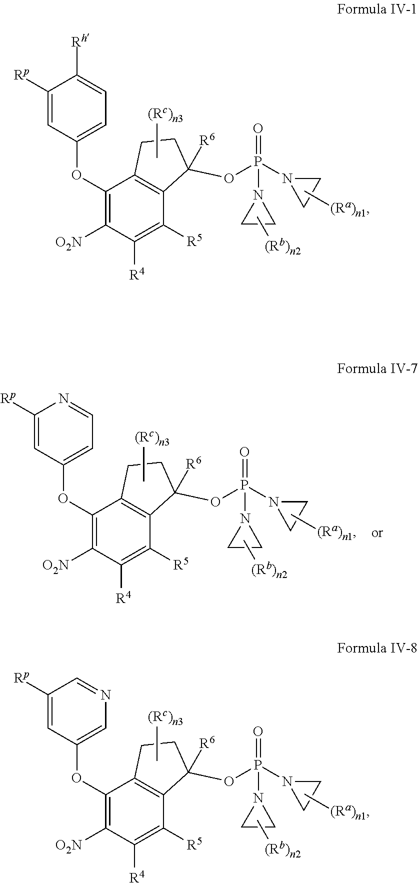

wherein:
R^{P} is halogen, CN, OH, NH₂, COOH, CONH₂, G³, OG³, SG₃, NHG³, NG³G³, C(O)G³, COOG³, CONHG³, CONG³G³, OC(O)G³, OCOOG³, OCONHG³, OCONG³G³, NHC(O)G³, NHCOOG³, NHCONHG³, NHCONG³G³, NG³C(O)G³, NG³COOG³, NG³CONHG³, NG³CONG³G³, SO₂G³, SO₂NHG³, or SO₂NG³G³, P G³ at each occurrence is independently an optionally substituted C₁₋₄ alkyl or optionally substituted 3-6 membered ring, such as cyclopropyl, cyclobutyl, oxetanyl

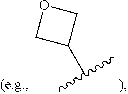 [SMILES: *C1COC1]

etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-7 membered (in additional embodiment, 4-8 membered) heterocyclic ring having 0 or 1 additional ring heteroatom, wherein the 4-7 membered (in additional embodiment, 4-8 membered) heterocyclic ring is optionally substituted with 1-3 substituents each independently oxo, F, C₁₋₄ alkyl, OH, NH₂, or C₁₋₄ heteroalkyl having 1-2 heteroatoms;
R^{h′} is hydrogen or R^{h}; and
R^{h} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

In some embodiments, each G³ is independently methyl,

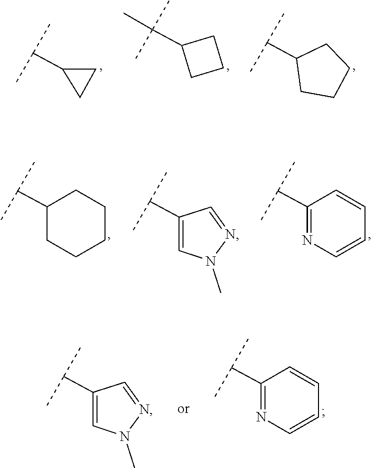

or two G³ together with the nitrogen atom they are both attached to are joined to form

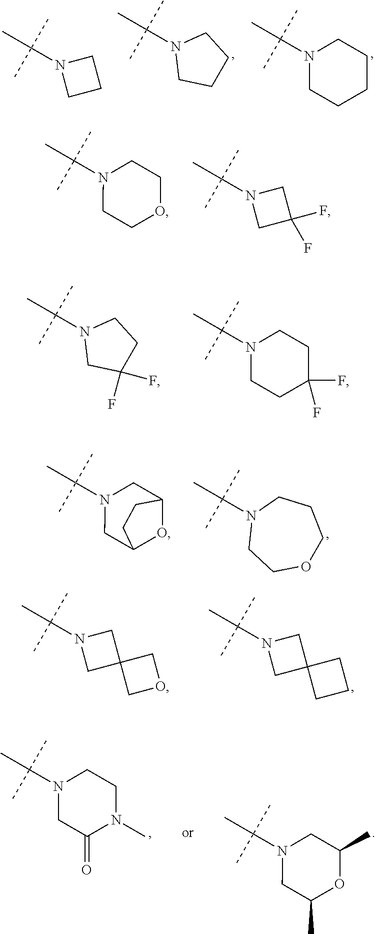

In one embodiment, each G³ is a C₁₋₄ alkyl such as methyl. In one embodiment, each G³ is a C₁₋₄ alkyl optionally substituted with F such as CF₃. For example, in some embodiments, R³ can be

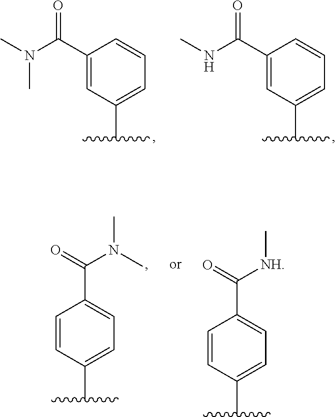

In some embodiments, R³ is

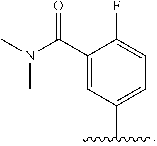 [SMILES: *c1ccc(F)c(C(=O)N(C)C)c1]

In some embodiments, each G³ is a deuterated analog of C₁₋₄ alkyl such as —CD₃. In some embodiments, R³ is

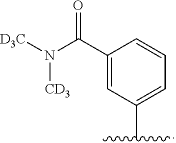 [SMILES: *c1cccc(C(=O)N(C([2H])([2H])[2H])C([2H])([2H])[2H])c1]

In some embodiments, R³ is

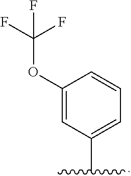 [SMILES: *c1cccc(OC(F)(F)F)c1]

In some embodiments, R³ is

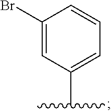 [SMILES: *c1cccc(Br)c1]

In some embodiments, R³ is

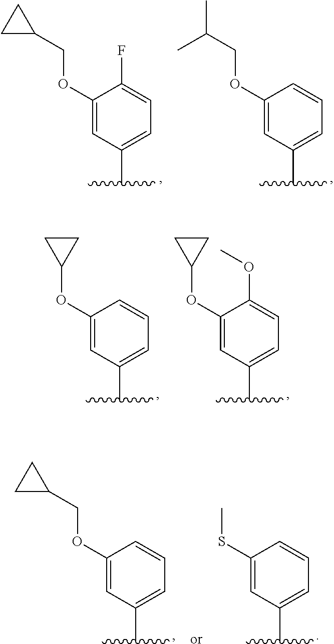

In some embodiments, NHG³ is NH-(cyclopropyl) or NH-(cyclobutyl). In some embodiments, in NG³G³, one instance of G³ is cyclopropyl or cyclobutyl and the other instance of G³ is a C₁₋₄ alkyl such as methyl. For example, in some embodiments, R³ can be

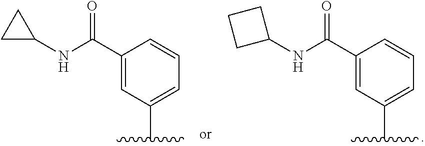 [SMILES: *c1cccc(C(=O)NC2CC2)c1 ; *c1cccc(C(=O)NC2CCC2)c1]

In some embodiments, NHG³ is NH-pyridyl, such as

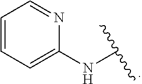 [SMILES: *Nc1ccccn1]

In some embodiments, in NG³G³, the two G³ together with the nitrogen atom they are both attached to are joined to form a 4-7 membered (in additional embodiment, 4-8 membered) heterocyclic ring having 0 or 1 additional ring heteroatom, such as an azetidine, pyrrolidine, morpholine, piperidine, piperazine, azepane, oxazepane ring, etc., wherein the 4-7 membered (in additional embodiment, 4-8 membered) heterocyclic ring is optionally substituted with 1-3 substituents as defined herein, for example, F, methyl, etc. For example, in some embodiments, R³ can be

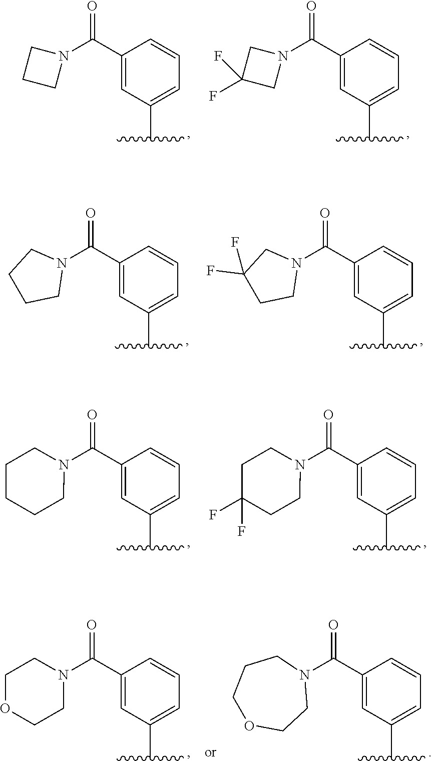

In some embodiments, R³ can be

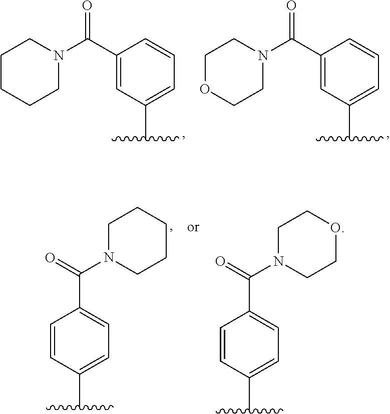

The integer n7 can be 0, i.e., the phenyl group of R³ is not further substituted (it also corresponds to: R^{h′} is hydrogen).

In some embodiments, the integer n7 can be 1, and R^{h} can be a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. For example, in some embodiments, R^{h} can be a halogen (e.g., F).

In some more specific embodiments, R³ in Formula I (e.g., any of the applicable subformulae herein) can be selected from:

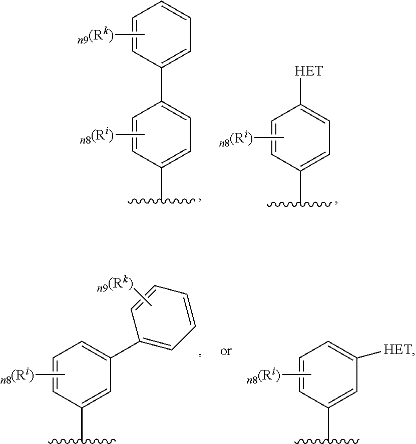

wherein:
the integers n8 and n9 are independently 0, 1, or 2;
HET is a 5 or 6 membered heterocyclyl or heteroaryl optionally substituted with 1-2 R^{j}; and
each of Rⁱ, R^{j}, and R^{k} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F or OH, or C₁₋₄ heteroalkyl having 1-2 heteroatoms and optionally substituted with F.

In one embodiment, the compound is a compound having a structure according to Formula IV-2, IV-3, IV-4, IV-5, IV-4-1, or IV-5-1, or a pharmaceutically acceptable salt thereof:

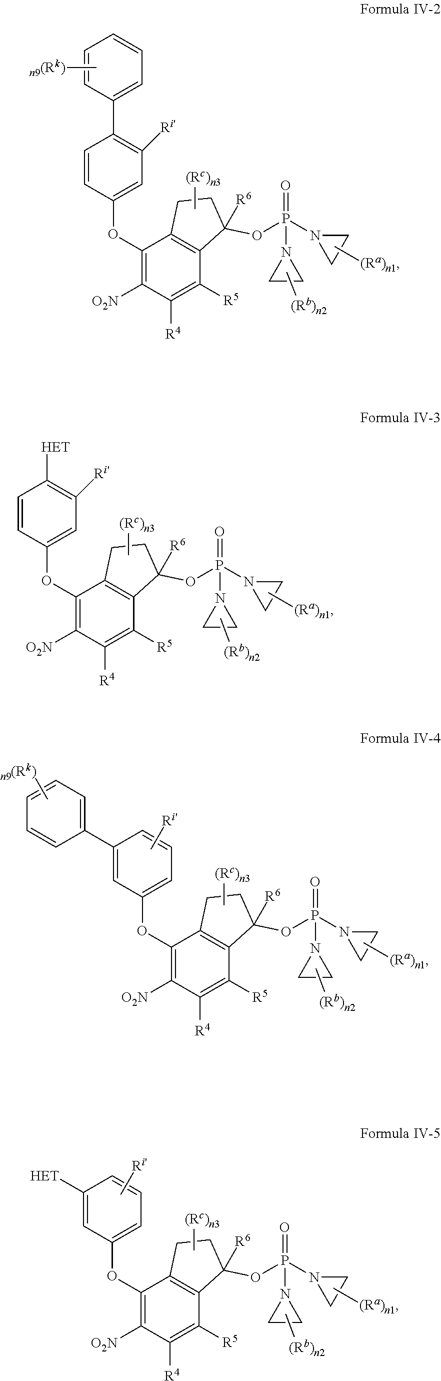

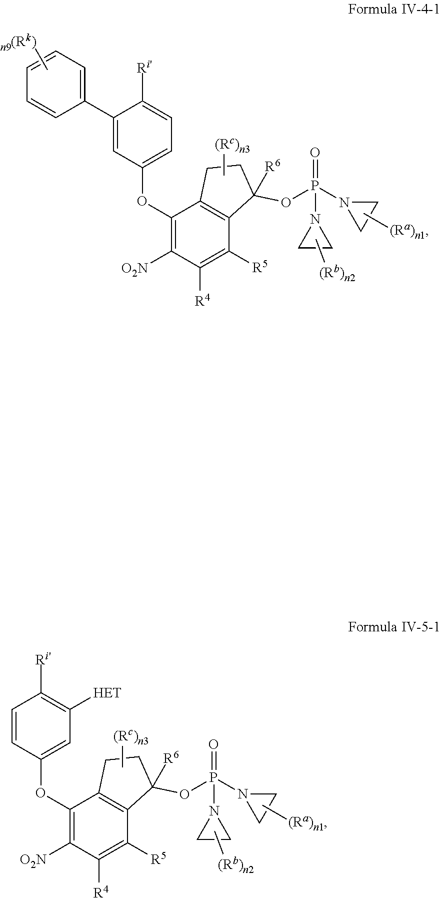

wherein R^{i′} is hydrogen or Rⁱ.

In some embodiments, the integer n8 is 0 (it also corresponds to: R^{i′} is hydrogen). In some embodiments, the integer n8 is 1 and Rⁱ is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. For example, in some embodiments, n8 is 1 and Rⁱ is a halogen (e.g., F).

In some embodiments, the integer n9 is 0. In some embodiments, the integer n9 is 1 and R^{k} is a halogen (e.g., F or Cl), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. For example, in some embodiments, n9 is 1 and R^{k} is a halogen (e.g., F or Cl) or C₁₋₄ alkoxy optionally substituted with F. In some embodiments,

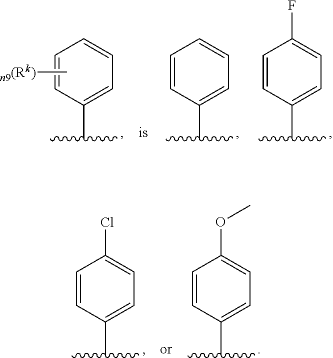

For example, in some embodiments, R³ in Formula I (e.g., any of the applicable subformulae herein) can be

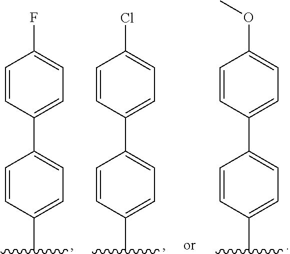

In some embodiments, R³ is

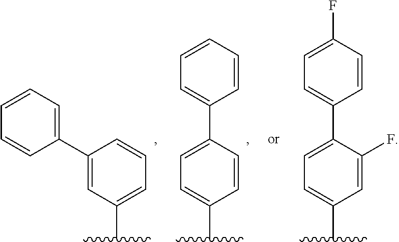

HET is typically a 5 or 6-membered heteroaryl. For example, in some embodiments, HET is a 6-membered heteroaryl having 1 or 2 ring nitrogen atoms, for example, a pyridyl (e.g., 2-pyridyl, 3-pyridyl, or 4-pyridyl) or pyrimidinyl, etc. In some embodiments, the 6-membered heteroaryl having 1 or 2 ring nitrogen atoms is optionally substituted with one R^{j}, which is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. In some embodiments, HET is a pyridyl

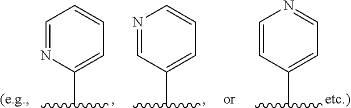

or pyrimidinyl

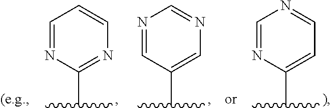

optionally substituted with 1-2 R^{j}, wherein R^{j} at each occurrence is independently F, OH, C₁₋₄ alkyl optionally substituted with F, or C₁₋₄ heteroalkyl having 1-2 heteroatoms and optionally substituted with F. In some embodiments, HET is pyrazinyl

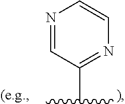 [SMILES: *c1cnccn1]

optionally substituted with 1-2 R^{j}, wherein R^{j} at each occurrence is independently F, OH, C₁₋₄ alkyl optionally substituted with F, or C₁₋₄ heteroalkyl having 1-2 heteroatoms and optionally substituted with F. For example, in some embodiments, R^{j} is F, CF₃, etc. In some embodiments, HET is

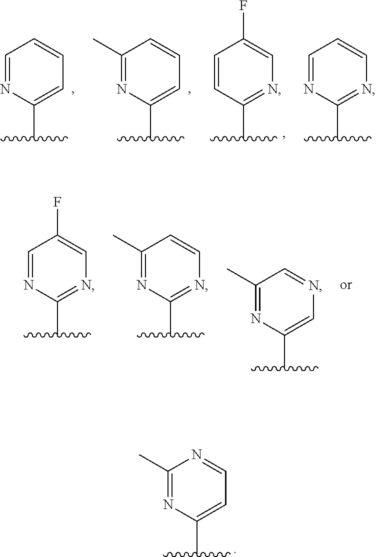

In some embodiments, R³ in Formula I (e.g., any of the applicable subformulae herein) can be

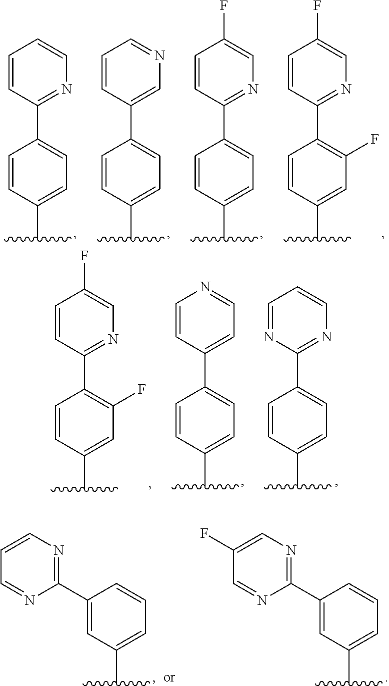

In some embodiments, R³ is

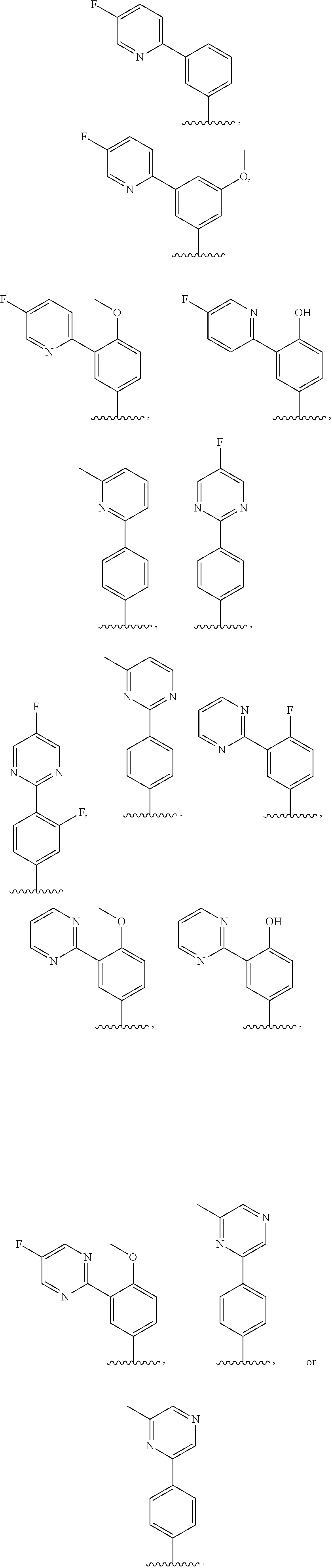

In some embodiments, HET is a pyridone

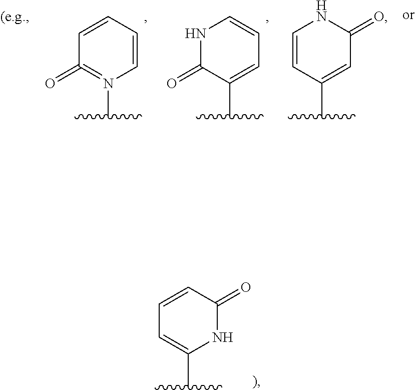

or pyrimidinone

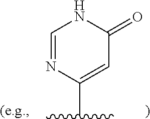 [SMILES: *c1cc(=O)[nH]cn1]

which is optionally substituted with one R^{j}, which is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. For example, in some embodiments, HET can be

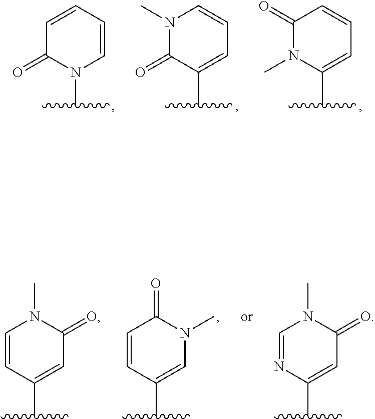

For example, in some embodiments, R³ in Formula I (e.g., any of the applicable subformulae herein) can be

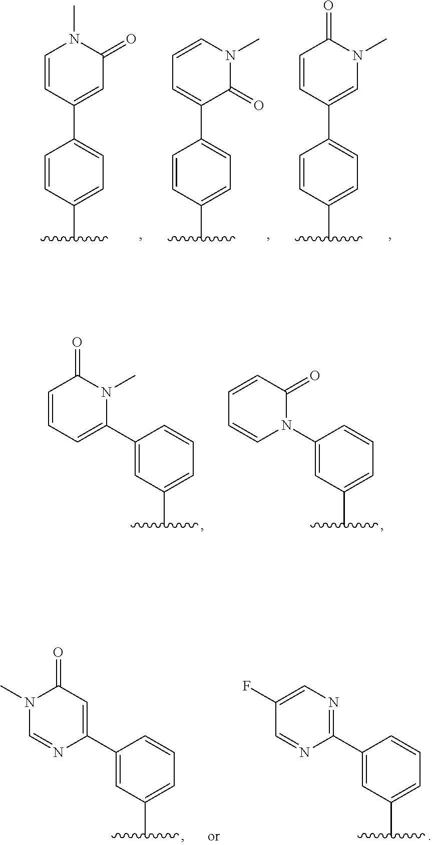

In some embodiments, HET is a 5-membered heteroaryl having 1-3 ring heteroatoms, for example, a pyrazole, oxadiazole, etc. In some embodiments, the 5-membered heteroaryl having 1-3 ring heteroatoms is optionally substituted with one R^{j}, which is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. In some embodiments, HET is a pyrazole or oxadiazole

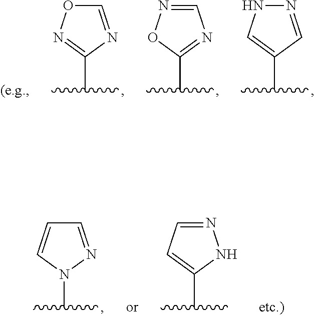

optionally substituted with 1-2 R^{j}, wherein R^{j} at each occurrence is independently F, OH, C₁₋₄ alkyl optionally substituted with F, C₁₋₄ heteroalkyl having 1-2 heteroatoms and optionally substituted with F or 3-4 membered ring such as cyclopropyl or cyclobutyl. In some embodiments, HET is a triazole

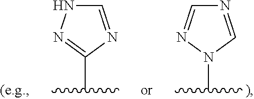 [SMILES: *c1nc[nH]n1 ; *n1cncn1]

tetrazole

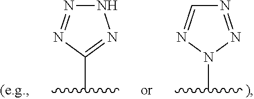 [SMILES: *c1nn[nH]n1 ; *n1ncnn1]

thiadiazole

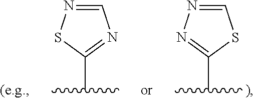 [SMILES: *c1ncns1 ; *c1nncs1]

thiazole

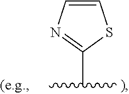 [SMILES: *c1nccs1]

oxazole

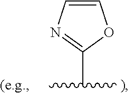 [SMILES: *c1ncco1]

or imidazole

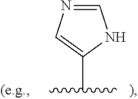 [SMILES: *c1cnc[nH]1]

optionally substituted with 1-2 R^{j}, wherein R^{j} at each occurrence is independently F, OH, C₁₋₄ alkyl optionally substituted with F, C₁₋₄ heteroalkyl having 1-2 heteroatoms and optionally substituted with F or 3-4 membered ring such as cyclopropyl or cyclobutyl. For example, in some embodiments, R^{j} is F, CH₃, CF₃, cyclopropyl, etc. For example, in some embodiments, HET can be

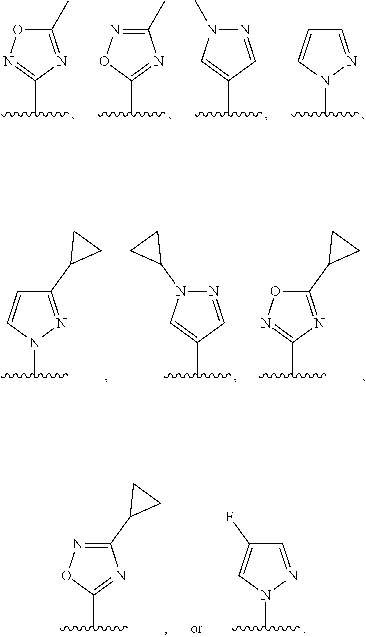

In some embodiments, HET is

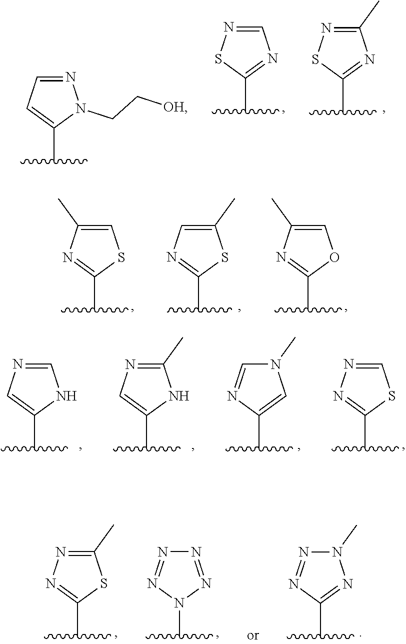

For example, in some embodiments, R³ in Formula I (e.g., any of the applicable subformulae herein) can be

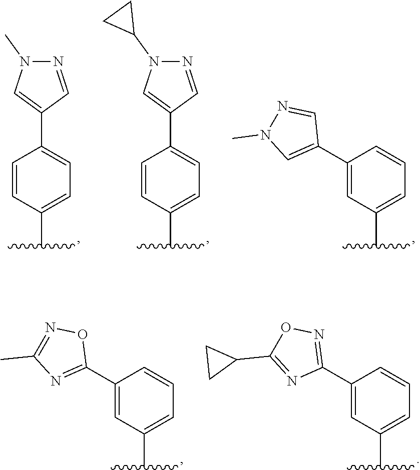

In some embodiments, R³ is

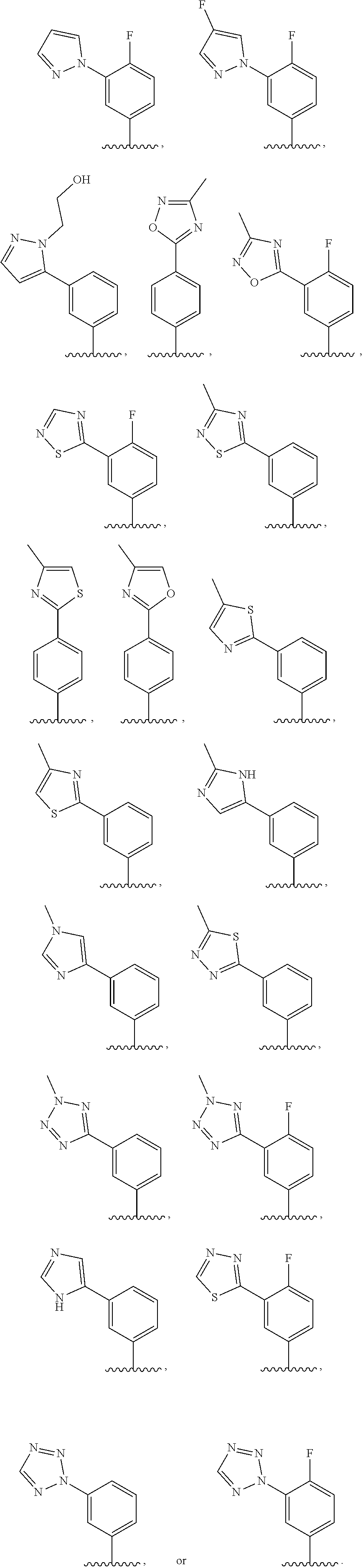

In some embodiments, HET is a 5 or 6-membered heterocyclyl having 1 or 2 ring heteroatoms, for example,

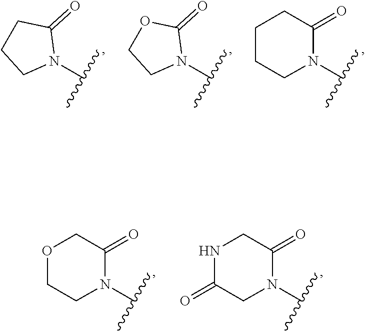 [SMILES: *N1CCCC1=O ; *N1CCOC1=O ; *N1CCCCC1=O ; *N1CCOCC1=O ; *N1CC(=O)NCC1=O]

etc. In some embodiments, the 5 or 6-membered heterocyclyl having 1 or 2 ring heteroatoms is optionally substituted with one or two R^{j}, which is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F. For example, in some embodiments, HET can be

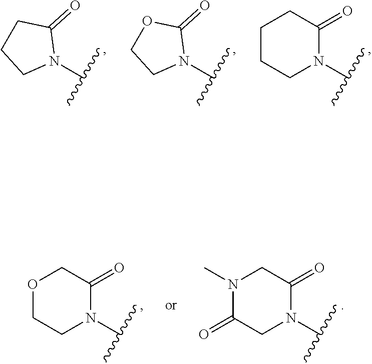

For example, in some embodiments, R³ in Formula I (e.g., any of the applicable subformulae herein) can be

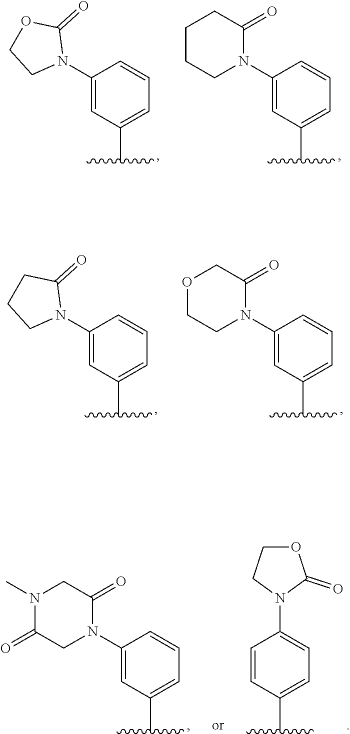

In some embodiments, R³ in Formula I (e.g., any of the applicable subformulae herein) can be

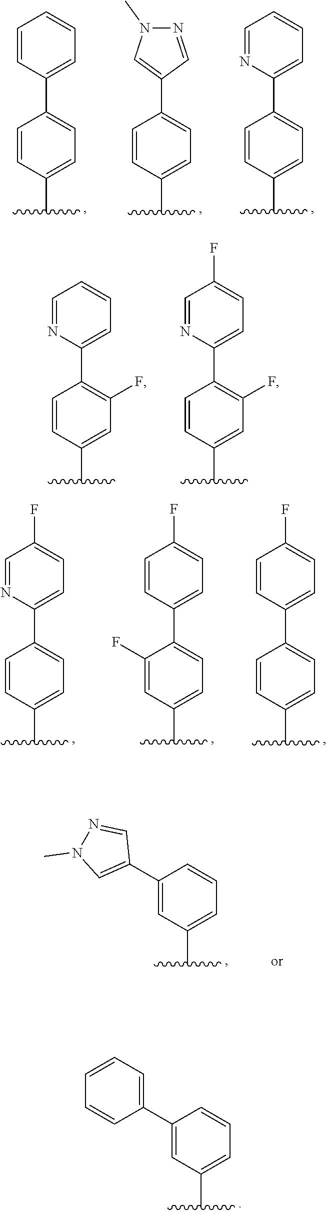

In some more specific embodiments, R³ in Formula I (e.g., any of the applicable subformulae herein) can be

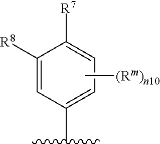

wherein:
the integer n10 is 0, 1, or 2; and
R⁷ and R⁸, together with the intervening atoms, are joined to form a 4-8 membered ring, which is optionally substituted with 1-3 Rⁿ; and
R^{m} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F; and
Rⁿ at each occurrence is independently oxo (as valency permits), halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

In one embodiment, the compound is a compound having a structure according to Formula IV-6, or a pharmaceutically acceptable salt thereof:

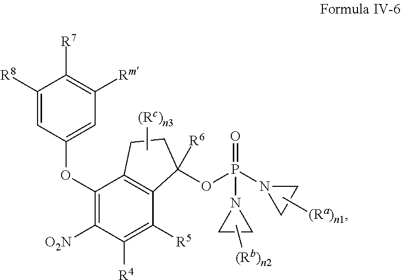

wherein R^{m′} is hydrogen or R^{m}.

The ring formed from R⁷ and R⁸ and the intervening atoms are not particularly limited. Typically, the ring formed is (1) a heterocyclic ring having one or two ring heteroatoms, for example, having one nitrogen, two nitrogens, or one nitrogen and one oxygen, or (2) a heteroaryl ring having 1-3 ring heteroatoms independently selected from O, N, and S. The ring formed may be optionally substituted with 1-3 Rⁿ as defined herein. When substituted, the Rⁿ can be attached to any available position as applicable.

In some embodiments, the ring formed from R⁷ and R⁸ and the intervening atoms a 5 or 6 membered heteroaryl ring having 1-3 ring heteroatoms, which is optionally substituted with 1-2 Rⁿ as defined herein. For example, in some embodiments, R³ can be

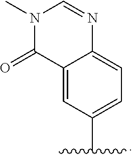 [SMILES: *c1ccc2ncn(C)c(=O)c2c1]

In some embodiments, R⁷ and R⁸, together with the intervening atoms, are joined to form a 4-7 membered heterocyclyl ring having 1 or 2 ring heteroatoms, such as one nitrogen, two nitrogens, two oxygens, or one nitrogen and one oxygen, which is optionally substituted with 1-2 Rⁿ as defined herein. For example, in some embodiments, R³ can be

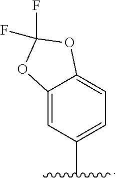 [SMILES: *c1ccc2c(c1)OC(F)(F)O2]

For example, in some more specific embodiments, R³ can be

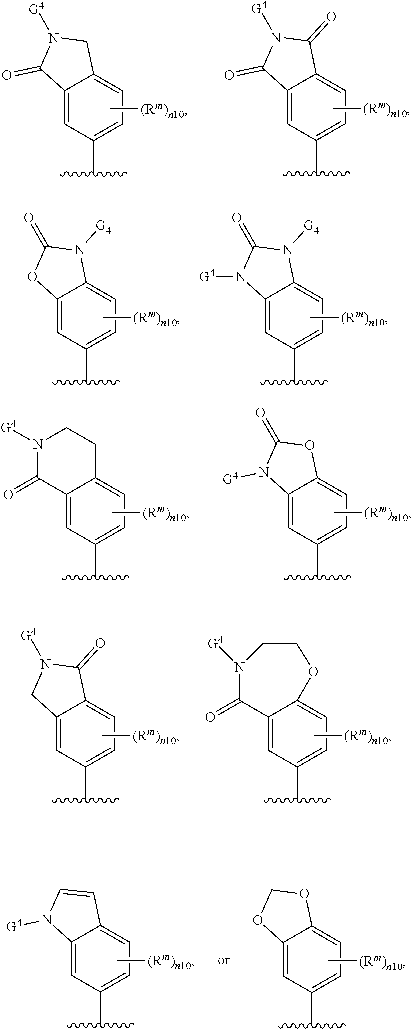

wherein:
R^{m} and n10 are defined herein, and
G⁴ at each occurrence is independently an optionally substituted C₁₋₄ alkyl, or optionally substituted 3-6 membered ring, such as cyclopropyl, cyclobutyl, oxetanyl

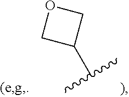 [SMILES: *C1COC1]

etc. When substituted, the C₁₋₄ alkyl or 3-6 membered ring is substituted with 1-3 substituents each independently F, OH, C₁₋₄ alkyl optionally substituted with F, or C₁₋₄ heteroalkyl having 1-2 heteroatoms and optionally substituted with F. For example, in some embodiments, G⁴ is methyl or where there are two G⁴, then both are methyl. In some embodiments, G⁴ is cyclopropyl or where there are two G⁴, one instance of G⁴ is cyclopropyl and the other instance of G⁴ is defined herein.

The integer n10 is typically 0 (it corresponds to: R^{m′} is hydrogen).

In some embodiments, n10 is 1, and R^{m} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

In some embodiments, R³ in Formula I (e.g., any of the applicable subformulae herein) can be as

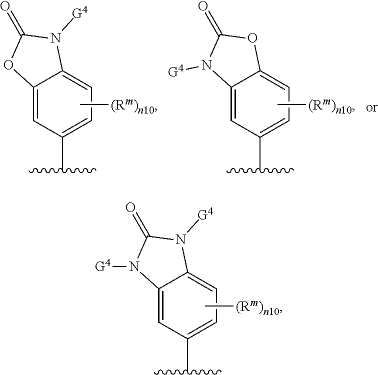

defined herein, for example,

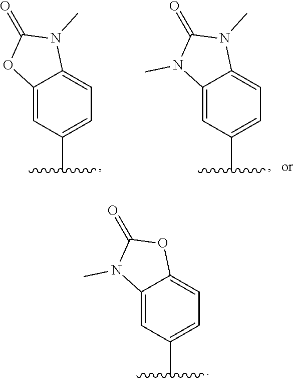 [SMILES: *c1ccc2c(c1)oc(=O)n2C ; *c1ccc2c(c1)n(C)c(=O)n2C ; *c1ccc2oc(=O)n(C)c2c1]

In some embodiments, R³ in Formula I (e.g., any of the applicable subformulae herein) can be

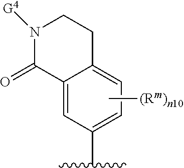

as defined herein, for example,

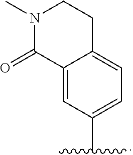 [SMILES: *c1ccc2c(c1)C(=O)N(C)CC2]

In some embodiments, R³ in Formula I (e.g., any of the applicable subformulae herein) can be

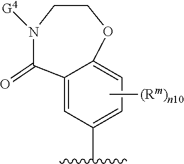

as defined herein, for example,

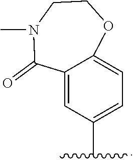 [SMILES: *c1ccc2c(c1)C(=O)N(C)CCO2]

In some embodiments, R³ in Formula I (e.g., any of the applicable subformulae herein) can be

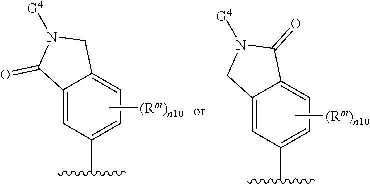

as defined herein. In some embodiments, R³ in Formula I (e.g., any of the applicable subformulae herein) can be

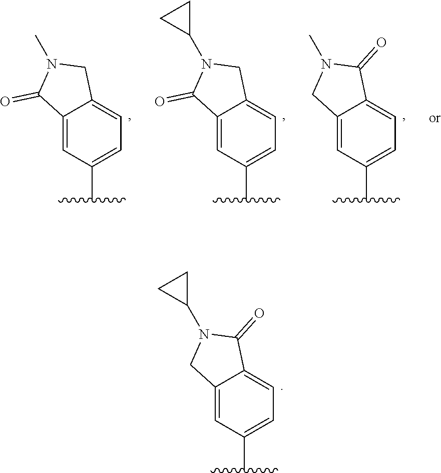

In some embodiments, R³ can be

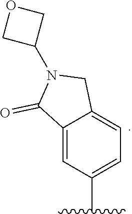 [SMILES: *c1ccc2c(c1)C(=O)N(C1COC1)C2]

In some embodiments, R³ in Formula I (e.g., any of the applicable subformulae herein) is

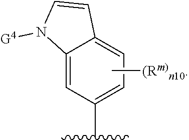

In some embodiments, R³ is

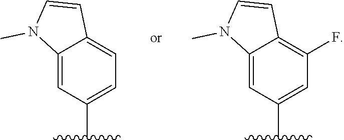 [SMILES: *c1ccc2ccn(C)c2c1 ; *c1cc(F)c2ccn(C)c2c1]

In some embodiments, R³ in Formula I (e.g., any of the applicable subformulae herein) is

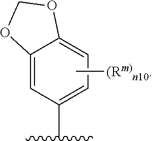

In some embodiments, R³ is

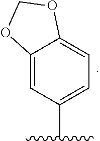 [SMILES: *c1ccc2c(c1)OCO2]

In some embodiments, the present disclosure also provides the following non-limiting exemplary embodiments A1-A14 according to Formula I.

Embodiment A1. A compound of Formula I, I-1, I-1-A, I-1-B, I-1-B-E1, I-1-B-E2, I-2, I-3, I-4, I-5, I-6, I-5-A, I-5-B, I-5-B-E1, I-5-B-E2, I-7, I-8, I-9, I-7-A, I-7-B, I-7-B-E1, I-7-B-E2, I-10, I-11, I-12, I-11A, I-11-B, I-11-B-E1, I-11-B-E2, I-13, I-13-B-E1, I-13-B-E2, I-13-B-E3, or I-13-B-E4, or a pharmaceutically acceptable salt thereof,
wherein the variables for each formula include any of those defined herein in any combination.

Embodiment A2. The compound of Embodiment A1, or a pharmaceutically acceptable salt thereof, wherein as applicable, R¹ is hydrogen or C₁₋₄ alkyl optionally substituted with F.

Embodiment A3. The compound of Embodiment A1, or a pharmaceutically acceptable salt thereof, wherein as applicable, R¹ is methyl or CF₃.

Embodiment A4. The compound of any of Embodiments A1-A3, or a pharmaceutically acceptable salt thereof, wherein as applicable, R² is hydrogen.

Embodiment A5. The compound of any of Embodiments A1-A4, or a pharmaceutically acceptable salt thereof, wherein as applicable, R⁴ is hydrogen.

Embodiment A6. The compound of any of Embodiments A1-A5, or a pharmaceutically acceptable salt thereof, wherein as applicable, R⁵ is hydrogen.

Embodiment A7. The compound of any of Embodiments A1-A6, or a pharmaceutically acceptable salt thereof, wherein as applicable, R⁶ is hydrogen.

Embodiment A8. The compound of any of Embodiments A1-A7, or a pharmaceutically acceptable salt thereof, wherein as applicable, n1 and n2 are both 0.

Embodiment A9. The compound of any of Embodiments A1-A7, or a pharmaceutically acceptable salt thereof, wherein as applicable, n1 and n2 are both 1, and R^{a} and R^{b} are both methyl.

Embodiment A10. The compound of any of Embodiments A1-A9, or a pharmaceutically acceptable salt thereof, wherein X is O.

Embodiment A11. The compound of any of Embodiments A1-A10, or a pharmaceutically acceptable salt thereof, wherein R³ is

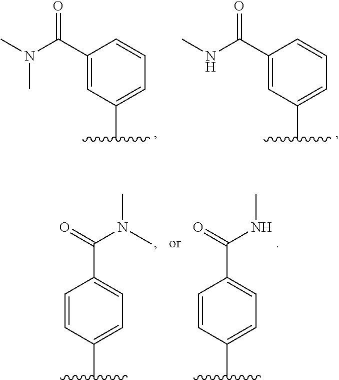

Embodiment A12. The compound of any of Embodiments A1-A10, or a pharmaceutically acceptable salt thereof, wherein R³ is

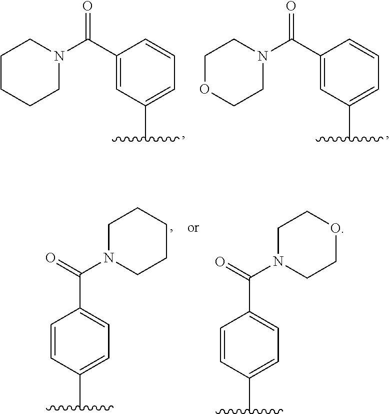

Embodiment A13. The compound of any of Embodiments A1-A10, or a pharmaceutically acceptable salt thereof, wherein R³ is

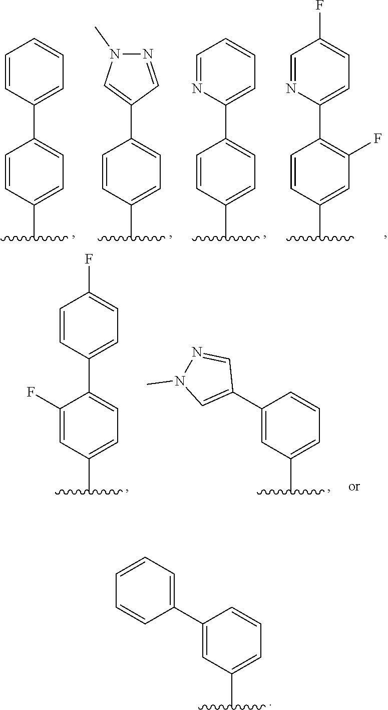

Embodiment A14. The compound of any of Embodiments A1-A10, or a pharmaceutically acceptable salt thereof, wherein R³ is

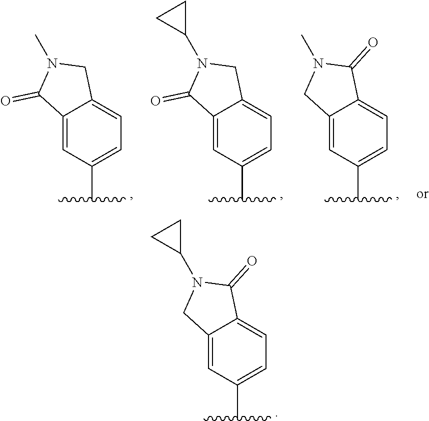

### Formula II

In some embodiments, the present disclosure provides a compound of Formula II, or a pharmaceutically acceptable salt thereof:

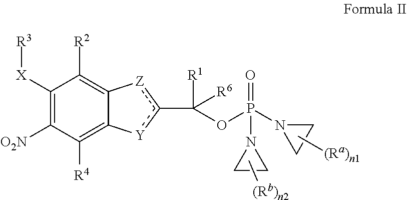

wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
Y and Z are each independently O, S, N, NR¹¹ or CR12, as valency permits, provided that the 5-membered ring containing Y and Z is aromatic; wherein:
   (i) R¹¹ is hydrogen, an optionally substituted C₁₋₄ alkyl, an optionally substituted 3-6 membered ring, or a nitrogen protecting group, R¹² is hydrogen, halogen, CN, an optionally substituted C₁₋₄ alkyl, an optionally substituted C₁₋₄ heteroalkyl, or an optionally substituted 3-6 membered ring; or
   (ii) R¹¹ or R¹², as applicable, together with R¹ and the intervening atoms are joined together to form an optionally substituted 4-8 membered ring (in one embodiment, an optionally substituted 5-8 membered ring);
R¹ is defined in (ii) or is hydrogen (in additional embodiment, deuterium), optionally substituted C₁₋₄ alkyl, optionally substituted C₂₋₄ alkenyl, or optionally substituted C₂₋₄ alkynyl;
R² and R⁴ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring;
R³ is hydrogen, an optionally substituted C₁₋₄ alkyl, or an optionally substituted 3-10 membered ring;
R⁶ is hydrogen (in additional embodiment, deuterium), optionally substituted C₁₋₄ alkyl, optionally substituted C₂₋₄ alkenyl, or optionally substituted C₂₋₄ alkynyl; the integers n1 and n2 are each independently 0, 1, 2, 3, or 4; and
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atoms, are joined together to form an optionally substituted 3-6 membered ring, and any remaining instances of R^{a} and/or R^{b} are as defined above.

In some embodiments, the compound of Formula II (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula II can exist in the form of an individual enantiomer and/or diastereomer, as applicable, or a mixture of stereoisomers, including racemic mixtures and mixtures enriched in one or more stereoisomers. In some embodiments, when applicable, the compound of Formula II (including any of the applicable sub-formulae as described herein) can exist as an individual enantiomer substantially free (e.g., with less than 20%, less than 10%, less than 5%, less than 1%, by weight, by HPLC or SFC area, or both, or with a non-detectable amount) of the other enantiomer, for example, the compound can have an enantiomeric excess (“ee”) of greater than 60%, such as 80% ee or greater, 90% ee or greater, 95% ee or greater, 98% ee or greater, 99% ee or greater, etc. In some embodiments, when applicable, the compound of Formula II (including any of the applicable sub-formulae as described herein) can also exist as a mixture of stereoisomers in any ratio, such as a racemic mixture.

In some embodiments, the compound of Formula II (including any of the applicable sub-formulae as described herein) can exist as an isotopically labeled compound, particularly, a deuterated analog, wherein one or more of the hydrogen atoms of the compound of Formula II is/are substituted with a deuterium atom with an abundance above its natural abundance, e.g., a CD₃ analog when the compound has a CH₃ group.

It should be apparent to those skilled in the art that in certain cases, the compound of Formula II may exist as a mixture of tautomers. The present disclosure is not limited to any specific tautomer. Rather, the present disclosure encompasses any and all of such tautomers whether or not explicitly drawn or referred to.

The Y and Z in Formula II are not particularly limited, so long as the 5-membered ring containing Y and Z is aromatic, for example, a furan, thiophene, pyrole, imidazole, etc.

In some embodiments, the 5-membered ring containing Y and Z in Formula II is a furan ring. For example, in some embodiments, the compound of Formula II can be characterized as having a structure according to Formula II-1:

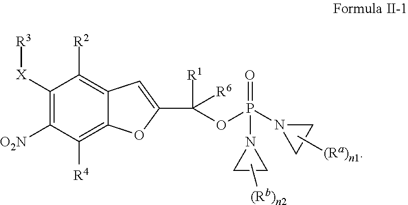

In one embodiment, the compound is a compound having a structure according to Formula V-1, or a pharmaceutically acceptable salt thereof:

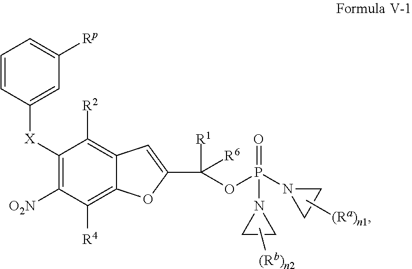

wherein:
R^{P} is halogen, CN, OH, NH₂, COOH, CONH₂, G³, OG³, SG₃, NHG³, NG³G³, C(O)G³, COOG³, CONHG³, CONG³G³, OC(O)G³, OCOOG³, OCONHG³, OCONG³G³, NHC(O)G³, NHCOOG³, NHCONHG³, NHCONG³G³, NG³C(O)G³, NG³COOG³, NG³CONHG³, NG³CONG³G³, SO₂G³, SO₂NHG³, or SO₂NG³G³,
G³ at each occurrence is independently an optionally substituted C₁₋₄ alkyl or optionally substituted 3-6 membered ring, such as cyclopropyl, cyclobutyl, oxetanyl

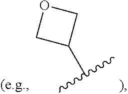 [SMILES: *C1COC1]

etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-7 membered (in additional embodiment, 4-8 membered) heterocyclic ring having 0 or 1 additional ring heteroatom, wherein the 4-7 membered (in additional embodiment, 4-8 membered) heterocyclic ring is optionally substituted with 1-3 substituents each independently oxo, F, C₁₋₄ alkyl, OH, NH₂, or C₁₋₄ heteroalkyl having 1-2 heteroatoms.

In some embodiments, R¹ in Formula II (e.g., II-1) can be hydrogen or C₁₋₄ alkyl optionally substituted with F, such as methyl, CF₃, etc. In one embodiment, R¹ is deuterium.

The variables R³, R⁶, X, R^{a}, R^{b}, n1, and n2 and any other variables (e.g., R¹, R², R⁴, G³, R^{h}, etc.) for Formula II or its subformulae (e.g., Formula II-1) can be any of those respective variable as defined herein for Formula I or its subformulae.

For example, typically, the integers n1 and n2 in Formula II are each independently 0, 1, or 2. In some embodiments, at least one of n1 and n2 is not 0. In some embodiments, the integers n1 and n2 in Formula II are both 0. In some embodiments, the integers n1 and n2 in Formula II are both 1. For example, in some embodiments, the compound of Formula II can be characterized as having a structure according to Formula II-2:

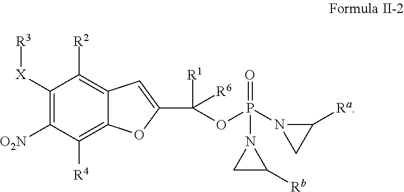

When n1 and n2 are both 1, R^{a} and R^{b} in Formula II (e.g., Formula II-1 or II-2) can be the same or different as defined herein. In some embodiments, n1 and n2 are both 1, R^{a} and R^{b} in Formula II are the same, which can be a C₁₋₄ alkyl, such as methyl, ethyl, or isopropyl.

Typically, R⁶ in Formula II (e.g., any of the applicable subformulae herein) is hydrogen. In one embodiment, R⁶ is deuterium. In one embodiment, R⁶ is optionally substituted C₁₋₄ alkyl. In one embodiment, R⁶ is CH₃. In one embodiment, R⁶ is CF₃. For example, in some embodiments, the compound of Formula II can be characterized as having a structure according to Formula II-3 or II-4:

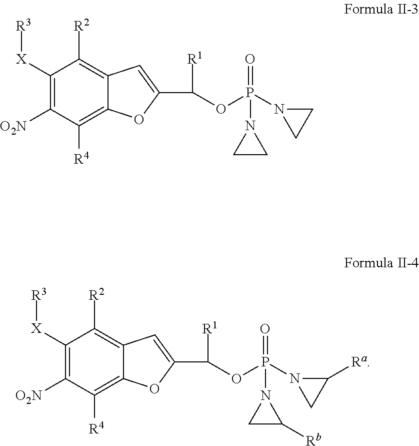

In some embodiments, the compound of Formula II can be characterized as having a structure according to Formula II-3-A or II-4-A:

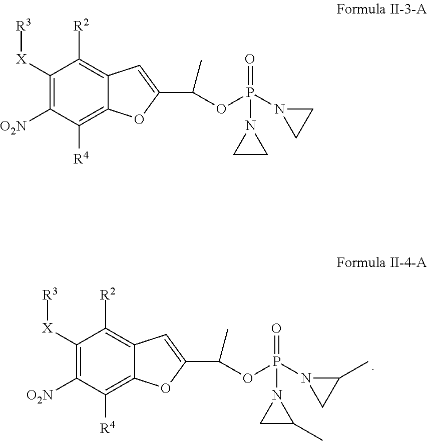

In some embodiments, the compound of Formula II can be characterized as having a structure according to Formula II-3-B or II-4-B:

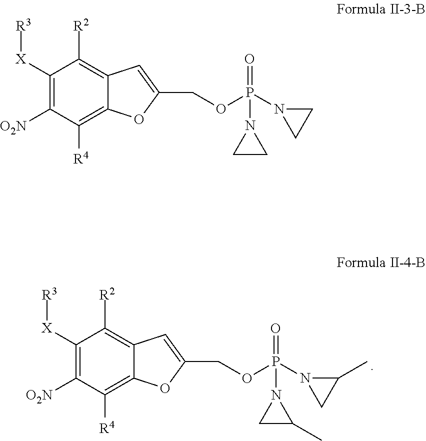

In some embodiments, the compound of Formula II-3-A can be a substantially pure enantiomer according to Formula II-3-A-E1:

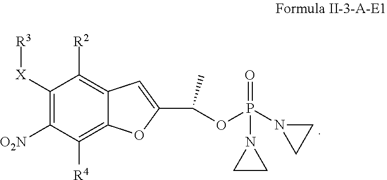

In some embodiments, the compound of Formula II-3-A can be a substantially pure enantiomer according to Formula II-3-A-E2:

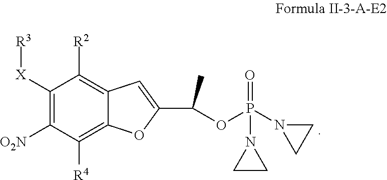

In some embodiments, the compound of Formula II-4-A can be a substantially pure enantiomer according to Formula II-4-A-E1:

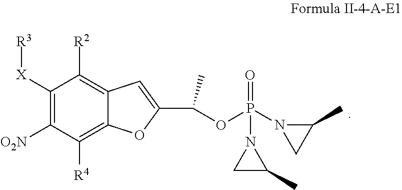

In some embodiments, the compound of Formula II-4-A can be a substantially pure enantiomer according to Formula II-4-A-E2:

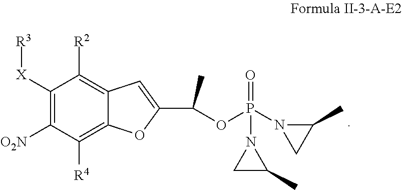

In some embodiments, the compound of Formula II-4-A can be a substantially pure enantiomer according to Formula II-4-A-E3:

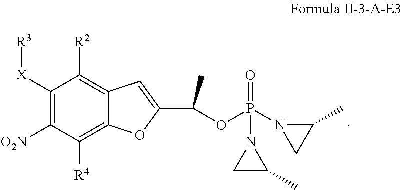

In some embodiments, the compound of Formula II-4-A can be a substantially pure enantiomer according to Formula II-4-A-E4:

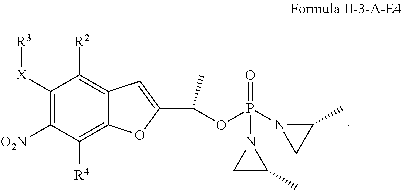

In some embodiments, the compound of Formula II-4-B can be a substantially pure enantiomer according to Formula II-4-B-E1:

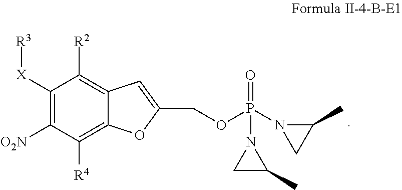

In some embodiments, the compound of Formula II-4-B can be a substantially pure enantiomer according to Formula II-4-B-E2:

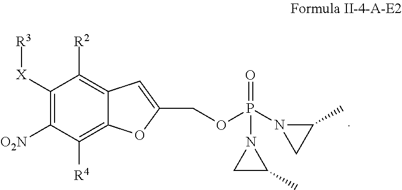

Typically, R⁴ in Formula II (e.g., any of the applicable subformulae herein) is hydrogen.

R² in Formula II (e.g., any of the applicable subformulae herein) is also typically hydrogen.

X in Formula II (e.g., any of the applicable subformulae herein) is typically O. In one embodiment, X is S. In one embodiment, X is NR¹⁰. In one embodiment, X is NH.

In some embodiments, X in Formula II (e.g., any of the applicable subformulae herein) can be a C₁₋₄ alkylene, such as CH₂.

In some embodiments, X in Formula II (e.g., any of the applicable subformulae herein) can be a C₁₋₄ heteroalkylene, such as those having one or two heteroatoms independently O, S, and N. For example, in some embodiments, X in Formula II (e.g., any of the applicable subformulae herein) can be —O—CH₂—. In one embodiment, X is —CH₂—O—. Unless otherwise specified, the left point of attachment of X is to R³.

R³ in Formula II (e.g., any of the applicable subformulae herein) can be any of those defined in connection with Formula I and its subformulae.

For example, in some embodiments, R³ in Formula II and its subformulae can be

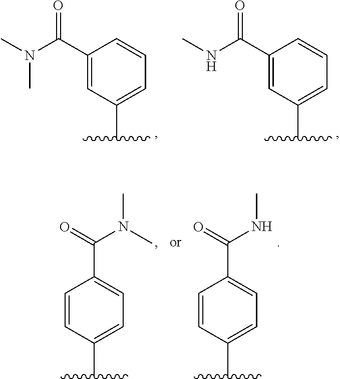

In some embodiments, R³ is

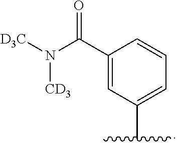 [SMILES: *c1cccc(C(=O)N(C([2H])([2H])[2H])C([2H])([2H])[2H])c1]

In some embodiments, R³ is

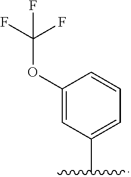 [SMILES: *c1cccc(OC(F)(F)F)c1]

In some embodiments, R³ is

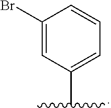 [SMILES: *c1cccc(Br)c1]

In some embodiments, R³ in Formula II and its subformulae can be

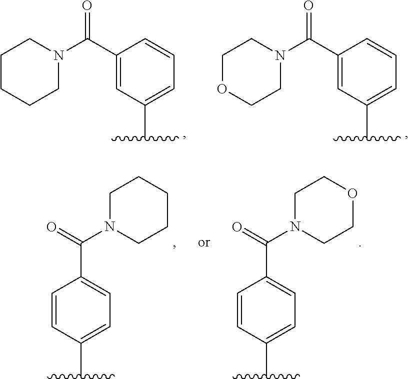

In some embodiments, R³ in Formula II (e.g., any of the applicable subformulae herein) can be

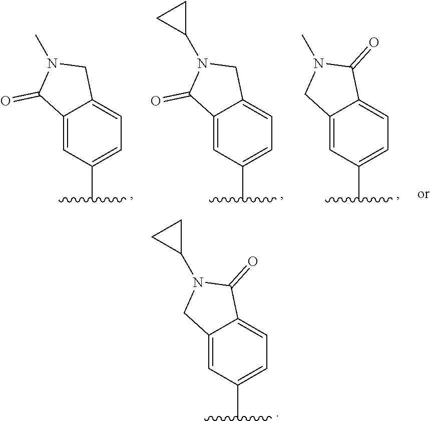

In some embodiments, R³ in Formula II (e.g., any of the applicable subformulae herein) can be

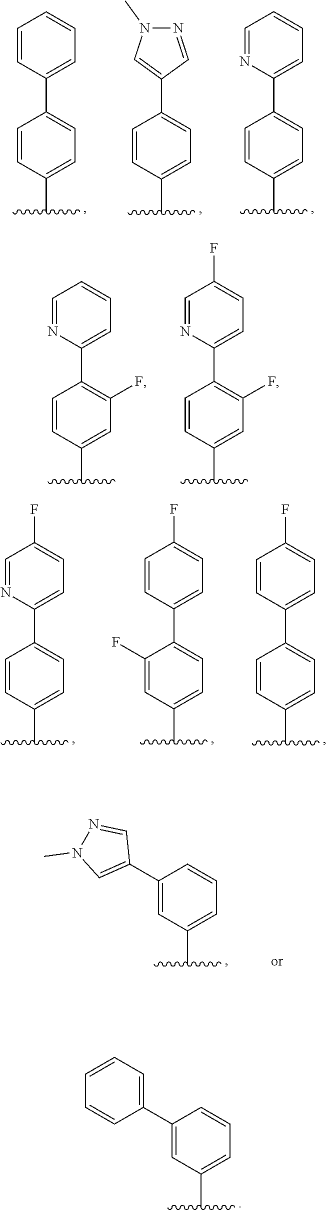

In some embodiments, the present disclosure also provides the following non-limiting exemplary embodiments B1-B14 according to Formula II.

Embodiment B1. A compound of Formula II, II-1, II-2, II-3, II-4, II-3-A, II-3-B, II-4-A, II-4-B, II-3-A-E1, II-3-A-E2, II-4-A-E1, II-4-A-E2, II-4-A-E3, II-4-A-E4, II-4-B-E1, II-4-B-E2, or a pharmaceutically acceptable salt thereof,
wherein the variables for each formula include any of those defined herein in any combination.

Embodiment B2. The compound of Embodiment B1, or a pharmaceutically acceptable salt thereof, wherein R¹ is hydrogen or C₁₋₄ alkyl optionally substituted with F.

Embodiment B3. The compound of Embodiment B1, or a pharmaceutically acceptable salt thereof, wherein R¹ is hydrogen.

Embodiment B4. The compound of Embodiment B1, or a pharmaceutically acceptable salt thereof, wherein as applicable, R¹ is methyl.

Embodiment B5. The compound of any of Embodiments B1-B4, or a pharmaceutically acceptable salt thereof, wherein R² is hydrogen.

Embodiment B6. The compound of any of Embodiments B1-B5, or a pharmaceutically acceptable salt thereof, wherein R⁴ is hydrogen.

Embodiment B7. The compound of any of Embodiments B1-B6, or a pharmaceutically acceptable salt thereof, wherein R⁶ is hydrogen.

Embodiment B8. The compound of any of Embodiments B1-B7, or a pharmaceutically acceptable salt thereof, wherein as applicable, n1 and n2 are both 0.

Embodiment B9. The compound of any of Embodiments B1-B7, or a pharmaceutically acceptable salt thereof, wherein as applicable, n1 and n2 are both 1, and R^{a} and R^{b} are both methyl.

Embodiment B10. The compound of any of Embodiments B1-B9, or a pharmaceutically acceptable salt thereof, wherein X is O.

Embodiment B11. The compound of any of Embodiments B1-B10, or a pharmaceutically acceptable salt thereof, wherein R³ is

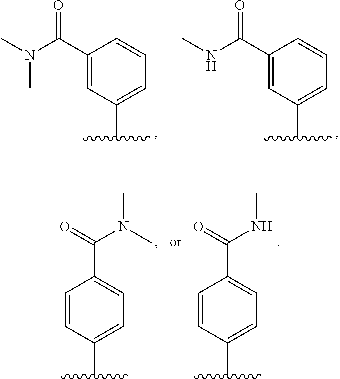

Embodiment B12. The compound of any of Embodiments B1-B10, or a pharmaceutically acceptable salt thereof, wherein R³ is

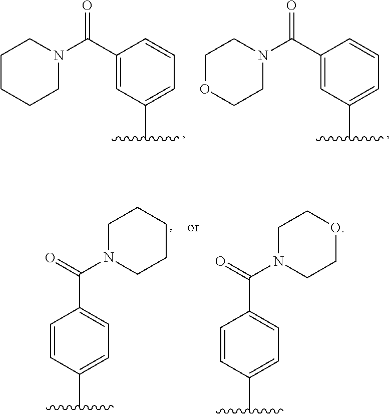

Embodiment B13. The compound of any of Embodiments B1-B10, or a pharmaceutically acceptable salt thereof, wherein R³ is

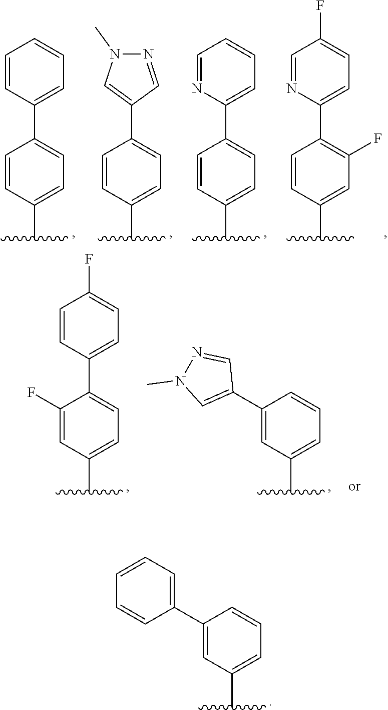

Embodiment B14. The compound of any of Embodiments B1-B10, or a pharmaceutically acceptable salt thereof, wherein R³ is

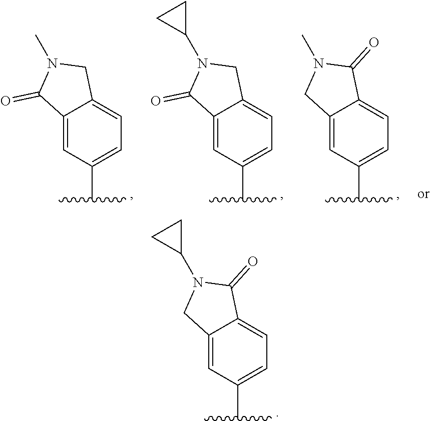

### Formula III

In some embodiments, the present disclosure provides a compound of Formula III, or a pharmaceutically acceptable salt thereof:

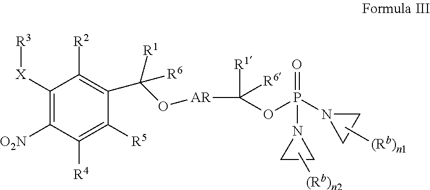

(1) R¹ is hydrogen (in additional embodiment, deuterium), optionally substituted C₁₋₄ alkyl, optionally substituted C₂₋₄ alkenyl, or optionally substituted C₂₋₄ alkynyl; and
   R², R⁴, and R⁵ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring; or
(2) R¹ and R², together with the intervening atoms, are joined to form an optionally substituted 4-8 membered carbocyclic or heterocyclic ring, and
   R⁴ and R⁵ are as defined in (1); or
(3) R¹ and R⁵, together with the intervening atoms, are joined to form an optionally substituted 4-8 membered carbocyclic or heterocyclic ring; and
   R² and R⁴ are as defined in (1); or
(4) R⁴ and R⁵, together with the intervening atoms, are joined to form an optionally substituted 4-8 membered ring; and
   R¹ and R² are as defined in (1) or (2); and
wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
AR represents an optionally substituted arylene or optionally substituted heteroarylene;
R³ is hydrogen, an optionally substituted C₁₋₄ alkyl, or an optionally substituted 3-10 membered ring;
R^{1′} is hydrogen (in additional embodiment, deuterium), optionally substituted C₁₋₄ alkyl, optionally substituted C₂₋₄ alkenyl, or optionally substituted C₂₋₄ alkynyl;
R⁶ and R^{6′} are each independently hydrogen (in additional embodiment, deuterium), optionally substituted C₁₋₄ alkyl, optionally substituted C₂₋₄ alkenyl, or optionally substituted C₂₋₄ alkynyl;
the integers n1 and n2 are each independently 0, 1, 2, 3, or 4; and
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atoms, are joined together to form an optionally substituted 3-6 membered ring, and any remaining instances of R^{a} and/or R^{b} are as defined above.

In some embodiments, the compound of Formula III (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula III can exist in the form of an individual enantiomer and/or diastereomer, as applicable, or a mixture of stereoisomers, including racemic mixtures and mixtures enriched in one or more stereoisomers. In some embodiments, when applicable, the compound of Formula III (including any of the applicable sub-formulae as described herein) can exist as an individual enantiomer substantially free (e.g., with less than 20%, less than 10%, less than 5%, less than 1%, by weight, by HPLC or SFC area, or both, or with a non-detectable amount) of the other enantiomer, for example, the compound can have an enantiomeric excess (“ee”) of greater than 60%, such as 80% ee or greater, 90% ee or greater, 95% ee or greater, 98% ee or greater, 99% ee or greater, etc. In some embodiments, when applicable, the compound of Formula III (including any of the applicable sub-formulae as described herein) can also exist as a mixture of stereoisomers in any ratio, such as a racemic mixture.

In some embodiments, the compound of Formula III (including any of the applicable sub-formulae as described herein) can exist as an isotopically labeled compound, particularly, a deuterated analog, wherein one or more of the hydrogen atoms of the compound of Formula III is/are substituted with a deuterium atom with an abundance above its natural abundance, e.g., a CD₃ analog when the compound has a CH₃ group.

It should be apparent to those skilled in the art that in certain cases, the compound of Formula III may exist as a mixture of tautomers. The present disclosure is not limited to any specific tautomer. Rather, the present disclosure encompasses any and all of such tautomers whether or not explicitly drawn or referred to.

The variables R¹, R², R³, R₄, R⁵, R⁶, X, R^{a}, R^{b}, n1, and n2 and any other variables for Formula III or its subformulae can be any of those respective variable as defined herein for Formula I or its subformulae. Further, the variable R^{1′} and R^{6′} can have any of the definitions of R¹ and R⁶, respectively, as defined herein for Formula I or its subformulae. In Formula III, R¹ and R^{1′} can be the same or different. Similarly, R⁶ and R^{6′} in Formula III can also be the same or different

In some embodiments, AR represents an optionally substituted phenylene. For example, AR can be a phenylene which is optionally substituted with 1-4 substituents independently selected from halogen, CN, OH, NH₂, optionally substituted C₁₋₄ alkyl, an optionally substituted C₂₋₄ alkenyl, an optionally substituted C₂₋₄ alkynyl, optionally substituted C₁₋₄ heteroalkyl, or an optionally substituted 3-6 membered ring structure, such as cyclopropyl, etc. In some embodiments, AR can be a tetrafluorophenylene,

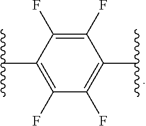 [SMILES: *c1c(F)c(F)c(*)c(F)c1F]

In some embodiments, AR represents an optionally substituted 5 or 6-membered heteroarylene. In some embodiments, AR can represent an optionally substituted bicyclic arylene or heteroarylene.

In some embodiments, the compound of Formula III can be characterized as having a structure according to Formula III-1, III-2, III-3, or III-4:

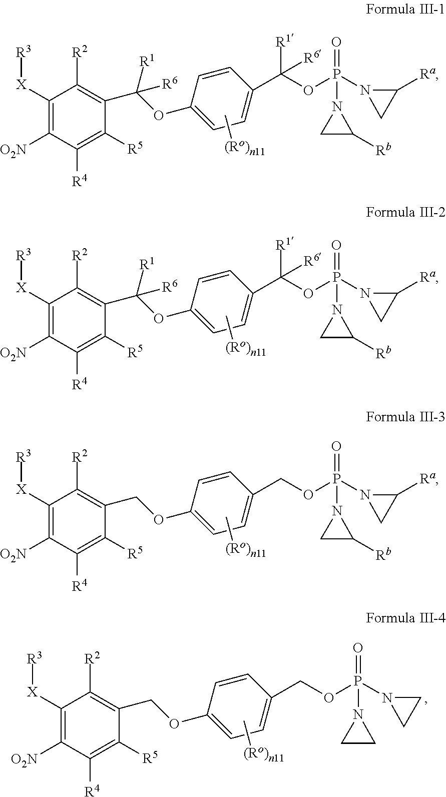

wherein:
n11 is 0, 1, 2, 3, or 4,
R^{o} at each occurrence is independently halogen (e.g., F), CN, OH, NH₂, optionally substituted C₁₋₄ alkyl, an optionally substituted C₂₋₄ alkenyl, an optionally substituted C₂₋₄ alkynyl, optionally substituted C₁₋₄ heteroalkyl, or an optionally substituted 3-6 membered ring structure, such as cyclopropyl, etc.; and
the other variables are defined herein.

In some embodiments, the integers n1 and n2 in Formula III are both 1, and R^{a} and R^{b} in Formula III (e.g., III-1, III-2, or III-3) are the same, which can be a C₁₋₄ alkyl, such as methyl, ethyl, or isopropyl.

Typically, R⁶ and R^{6′} in Formula III (e.g., any of the applicable subformulae herein) are both hydrogen. In one embodiment, R⁶ is deuterium. In one embodiment, R^{6′} is deuterium.

In some embodiments, R¹ and R^{1′} in Formula III (e.g., any of the applicable subformulae herein) are independently hydrogen or methyl, such as both being hydrogen or both being methyl. In one embodiment, R¹ is deuterium. In one embodiment, R^{1′} is deuterium.

In some embodiments, R⁴ and R⁵ in Formula III (e.g., any of the applicable subformulae herein) are both hydrogen.

In some embodiments, R² in Formula III (e.g., any of the applicable subformulae herein) is hydrogen.

X in Formula III (e.g., any of the applicable subformulae herein) is typically O. In one embodiment, X is S. In one embodiment, X is NR¹⁰. In one embodiment, X is NH.

In some embodiments, X in Formula III (e.g., any of the applicable subformulae herein) can be a C₁₋₄ alkylene, such as CH₂.

In some embodiments, X in Formula III (e.g., any of the applicable subformulae herein) can be a C₁₋₄ heteroalkylene, such as those having one or two heteroatoms independently O, S, and N. For example, in some embodiments, X in Formula III (e.g., any of the applicable subformulae herein) can be —O—CH₂—. In one embodiment, X is —CH₂—O—. Unless otherwise specified, the left point of attachment of X is to R³.

R³ in Formula III (e.g., any of the applicable subformulae herein) can be any of those defined in connection with Formula I and its subformulae.

For example, in some embodiments, R³ in Formula III and its subformulae can be

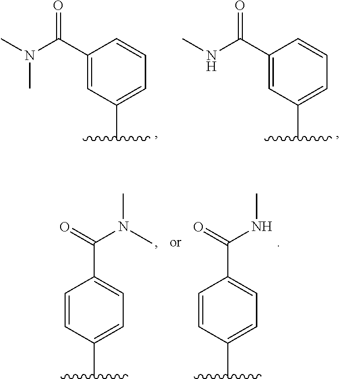

In some embodiments, R³ in Formula III and its subformulae can be

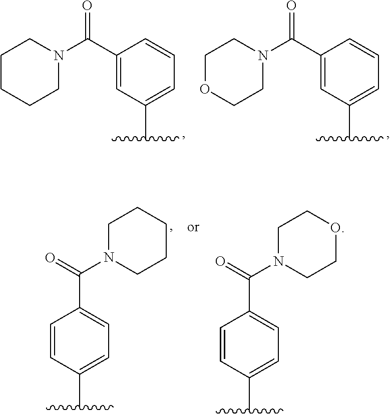

In some embodiments, R³ in Formula III (e.g., any of the applicable subformulae herein) can be

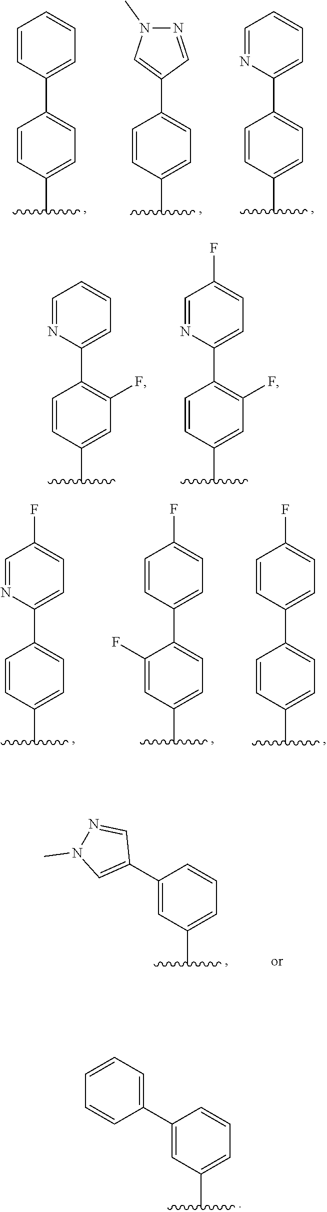

In one embodiment of any applicable formula provided herein, the carbon connected to R⁶ has S-configuration. In one embodiment of any applicable formula provided herein, the carbon connected to R⁶ has R-configuration. In one embodiment of any applicable formula provided herein, the carbon connected to R^{6′} has S-configuration. In one embodiment of any applicable formula provided herein, the carbon connected to R^{6′} has R-configuration.

In some embodiments, the present disclosure provides a compound selected from any of the following shown in Tables A1 to A10, or a pharmaceutically acceptable salt thereof:

TABLE Al
List compounds

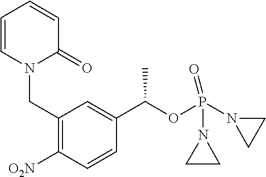 [SMILES: C[C@H](OP(=O)(N1CC1)N1CC1)c1ccc([N+](=O)[O-])c(Cn2ccccc2=O)c1]

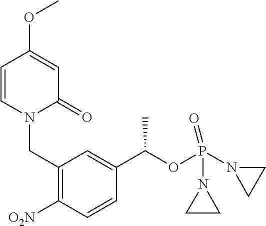 [SMILES: COc1ccn(Cc2cc([C@H](C)OP(=O)(N3CC3)N3CC3)ccc2[N+](=O)[O-])c(=O)c1]

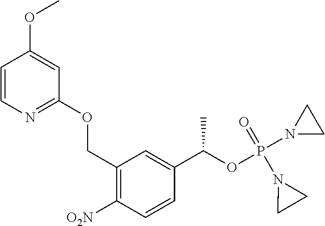 [SMILES: COc1ccnc(OCc2cc([C@H](C)OP(=O)(N3CC3)N3CC3)ccc2[N+](=O)[O-])c1]

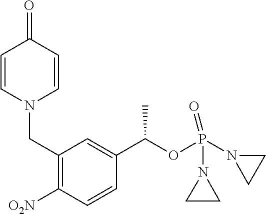 [SMILES: C[C@H](OP(=O)(N1CC1)N1CC1)c1ccc([N+](=O)[O-])c(Cn2ccc(=O)cc2)c1]

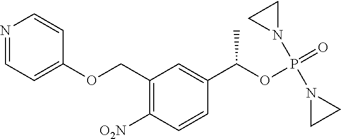 [SMILES: C[C@H](OP(=O)(N1CC1)N1CC1)c1ccc([N+](=O)[O-])c(COc2ccncc2)c1]

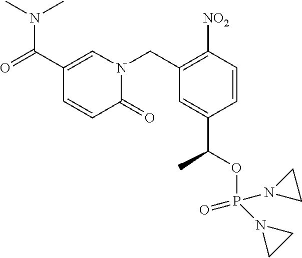 [SMILES: C[C@H](OP(=O)(N1CC1)N1CC1)c1ccc([N+](=O)[O-])c(Cn2cc(C(=O)N(C)C)ccc2=O)c1]

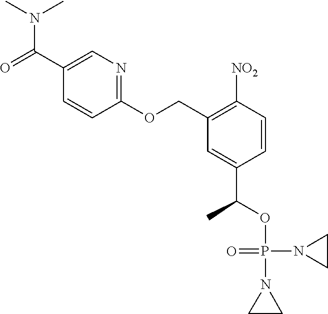 [SMILES: C[C@H](OP(=O)(N1CC1)N1CC1)c1ccc([N+](=O)[O-])c(COc2ccc(C(=O)N(C)C)cn2)c1]

TABLE A2
List of compounds

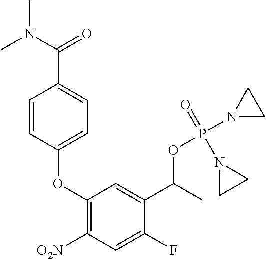 [SMILES: CC(OP(=O)(N1CC1)N1CC1)c1cc(Oc2ccc(C(=O)N(C)C)cc2)c([N+](=O)[O-])cc1F]

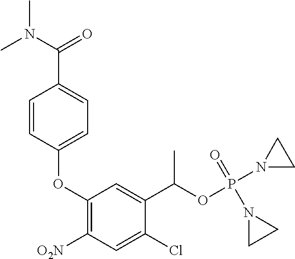 [SMILES: CC(OP(=O)(N1CC1)N1CC1)c1cc(Oc2ccc(C(=O)N(C)C)cc2)c([N+](=O)[O-])cc1Cl]

TABLE A3
List of compounds

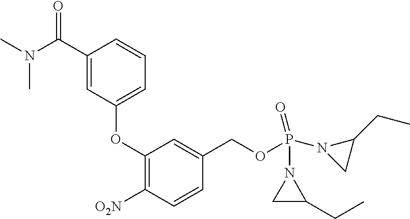 [SMILES: CCC1CN1P(=O)(OCc1ccc([N+](=O)[O-])c(Oc2cccc(C(=O)N(C)C)c2)c1)N1CC1CC]

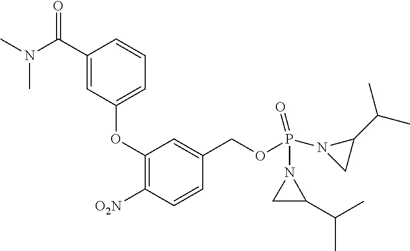 [SMILES: CC(C)C1CN1P(=O)(OCc1ccc([N+](=O)[O-])c(Oc2cccc(C(=O)N(C)C)c2)c1)N1CC1C(C)C]

TABLE A4
List of compounds

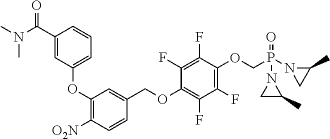

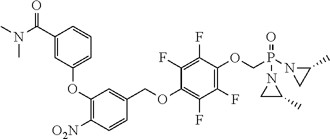

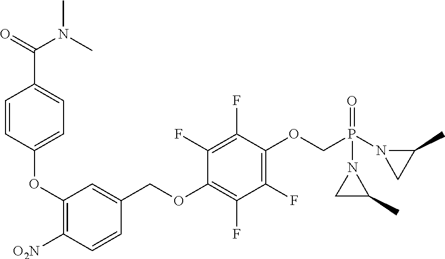 [SMILES: C[C@H]1CN1P(=O)(COc1c(F)c(F)c(OCc2ccc([N+](=O)[O-])c(Oc3ccc(C(=O)N(C)C)cc3)c2)c(F)c1F)N1C[C@@H]1C]

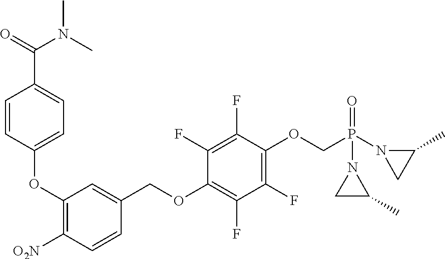 [SMILES: C[C@@H]1CN1P(=O)(COc1c(F)c(F)c(OCc2ccc([N+](=O)[O-])c(Oc3ccc(C(=O)N(C)C)cc3)c2)c(F)c1F)N1C[C@H]1C]

TABLE A5
List of compounds

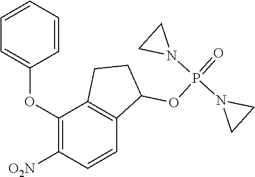 [SMILES: O=[N+]([O-])c1ccc2c(c1Oc1ccccc1)CCC2OP(=O)(N1CC1)N1CC1]

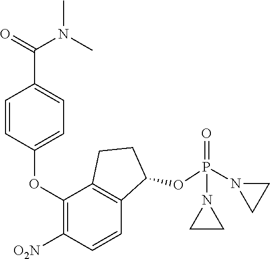 [SMILES: CN(C)C(=O)c1ccc(Oc2c([N+](=O)[O-])ccc3c2CC[C@@H]3OP(=O)(N2CC2)N2CC2)cc1]

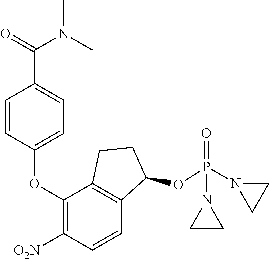 [SMILES: CN(C)C(=O)c1ccc(Oc2c([N+](=O)[O-])ccc3c2CC[C@H]3OP(=O)(N2CC2)N2CC2)cc1]

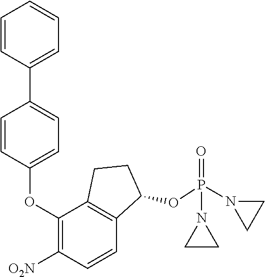 [SMILES: O=[N+]([O-])c1ccc2c(c1Oc1ccc(-c3ccccc3)cc1)CC[C@@H]2OP(=O)(N1CC1)N1CC1]

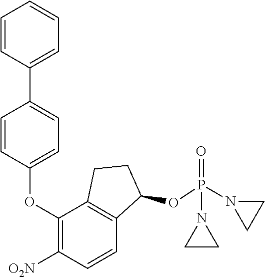 [SMILES: O=[N+]([O-])c1ccc2c(c1Oc1ccc(-c3ccccc3)cc1)CC[C@H]2OP(=O)(N1CC1)N1CC1]

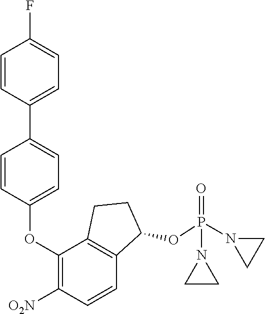 [SMILES: O=[N+]([O-])c1ccc2c(c1Oc1ccc(-c3ccc(F)cc3)cc1)CC[C@@H]2OP(=O)(N1CC1)N1CC1]

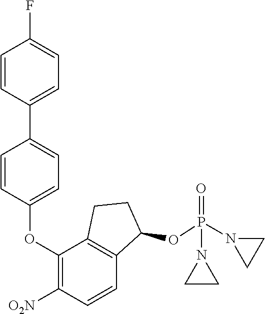 [SMILES: O=[N+]([O-])c1ccc2c(c1Oc1ccc(-c3ccc(F)cc3)cc1)CC[C@H]2OP(=O)(N1CC1)N1CC1]

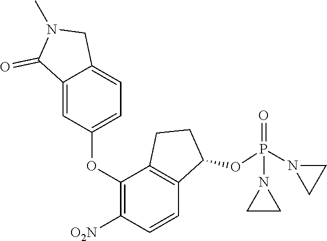 [SMILES: CN1Cc2ccc(Oc3c([N+](=O)[O-])ccc4c3CC[C@@H]4OP(=O)(N3CC3)N3CC3)cc2C1=O]

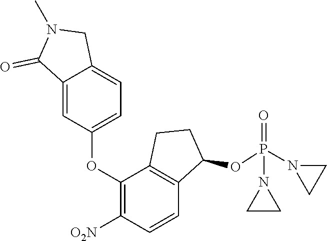 [SMILES: CN1Cc2ccc(Oc3c([N+](=O)[O-])ccc4c3CC[C@H]4OP(=O)(N3CC3)N3CC3)cc2C1=O]

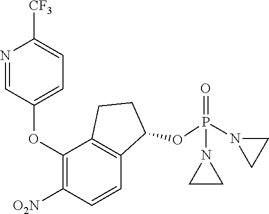 [SMILES: O=[N+]([O-])c1ccc2c(c1Oc1ccc(C(F)(F)F)nc1)CC[C@@H]2OP(=O)(N1CC1)N1CC1]

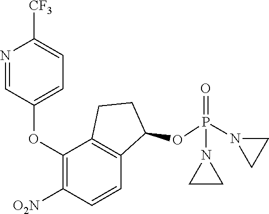 [SMILES: O=[N+]([O-])c1ccc2c(c1Oc1ccc(C(F)(F)F)nc1)CC[C@H]2OP(=O)(N1CC1)N1CC1]

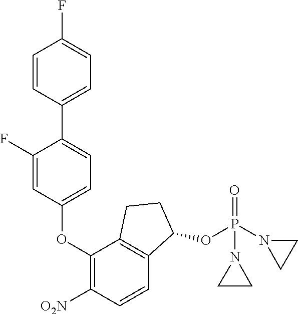 [SMILES: O=[N+]([O-])c1ccc2c(c1Oc1ccc(-c3ccc(F)cc3)c(F)c1)CC[C@@H]2OP(=O)(N1CC1)N1CC1]

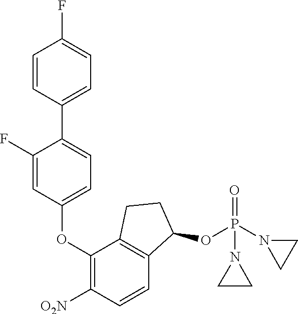 [SMILES: O=[N+]([O-])c1ccc2c(c1Oc1ccc(-c3ccc(F)cc3)c(F)c1)CC[C@H]2OP(=O)(N1CC1)N1CC1]

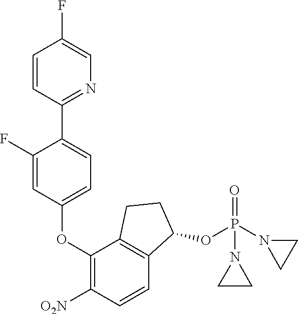 [SMILES: O=[N+]([O-])c1ccc2c(c1Oc1ccc(-c3ccc(F)cn3)c(F)c1)CC[C@@H]2OP(=O)(N1CC1)N1CC1]

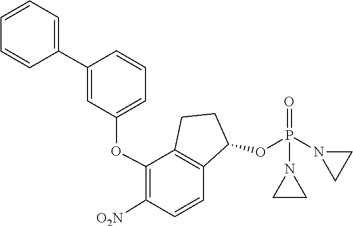 [SMILES: O=[N+]([O-])c1ccc2c(c1Oc1cccc(-c3ccccc3)c1)CC[C@@H]2OP(=O)(N1CC1)N1CC1]

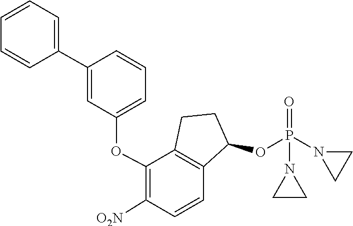 [SMILES: O=[N+]([O-])c1ccc2c(c1Oc1cccc(-c3ccccc3)c1)CC[C@H]2OP(=O)(N1CC1)N1CC1]

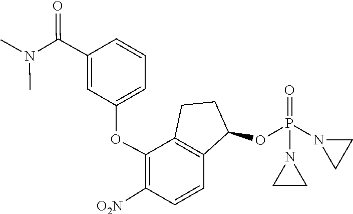 [SMILES: CN(C)C(=O)c1cccc(Oc2c([N+](=O)[O-])ccc3c2CC[C@H]3OP(=O)(N2CC2)N2CC2)c1]

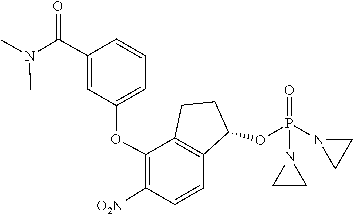 [SMILES: CN(C)C(=O)c1cccc(Oc2c([N+](=O)[O-])ccc3c2CC[C@@H]3OP(=O)(N2CC2)N2CC2)c1]

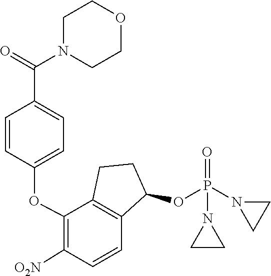 [SMILES: O=C(c1ccc(Oc2c([N+](=O)[O-])ccc3c2CC[C@H]3OP(=O)(N2CC2)N2CC2)cc1)N1CCOCC1]

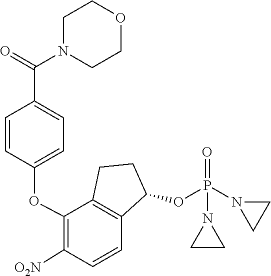 [SMILES: O=C(c1ccc(Oc2c([N+](=O)[O-])ccc3c2CC[C@@H]3OP(=O)(N2CC2)N2CC2)cc1)N1CCOCC1]

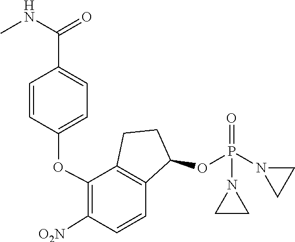 [SMILES: CNC(=O)c1ccc(Oc2c([N+](=O)[O-])ccc3c2CC[C@H]3OP(=O)(N2CC2)N2CC2)cc1]

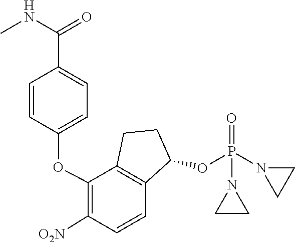 [SMILES: CNC(=O)c1ccc(Oc2c([N+](=O)[O-])ccc3c2CC[C@@H]3OP(=O)(N2CC2)N2CC2)cc1]

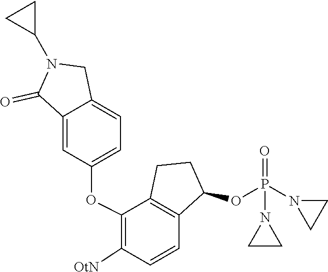

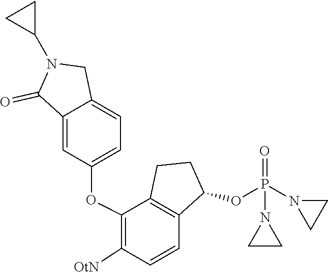

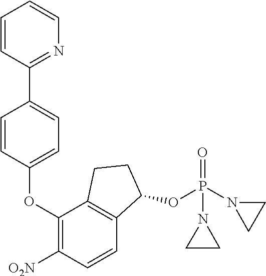 [SMILES: O=[N+]([O-])c1ccc2c(c1Oc1ccc(-c3ccccn3)cc1)CC[C@@H]2OP(=O)(N1CC1)N1CC1]

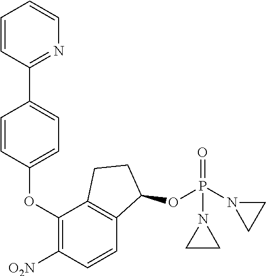 [SMILES: O=[N+]([O-])c1ccc2c(c1Oc1ccc(-c3ccccn3)cc1)CC[C@H]2OP(=O)(N1CC1)N1CC1]

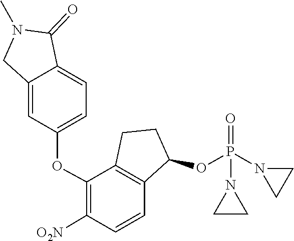 [SMILES: CN1Cc2cc(Oc3c([N+](=O)[O-])ccc4c3CC[C@H]4OP(=O)(N3CC3)N3CC3)ccc2C1=O]

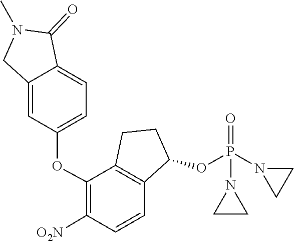 [SMILES: CN1Cc2cc(Oc3c([N+](=O)[O-])ccc4c3CC[C@@H]4OP(=O)(N3CC3)N3CC3)ccc2C1=O]

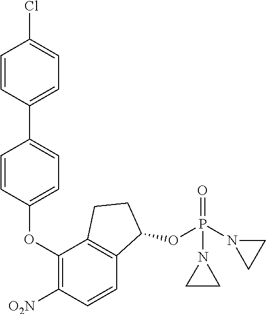 [SMILES: O=[N+]([O-])c1ccc2c(c1Oc1ccc(-c3ccc(Cl)cc3)cc1)CC[C@@H]2OP(=O)(N1CC1)N1CC1]

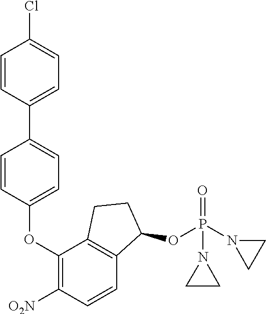 [SMILES: O=[N+]([O-])c1ccc2c(c1Oc1ccc(-c3ccc(Cl)cc3)cc1)CC[C@H]2OP(=O)(N1CC1)N1CC1]

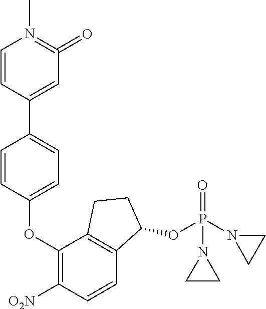

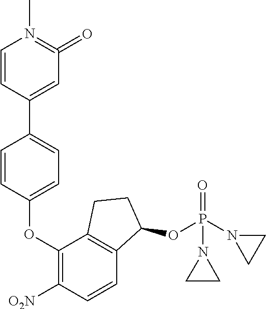

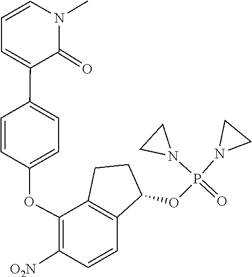 [SMILES: Cn1cccc(-c2ccc(Oc3c([N+](=O)[O-])ccc4c3CC[C@@H]4OP(=O)(N3CC3)N3CC3)cc2)c1=O]

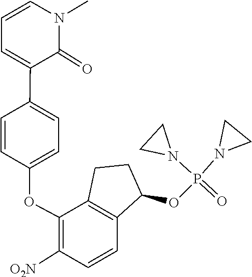 [SMILES: Cn1cccc(-c2ccc(Oc3c([N+](=O)[O-])ccc4c3CC[C@H]4OP(=O)(N3CC3)N3CC3)cc2)c1=O]

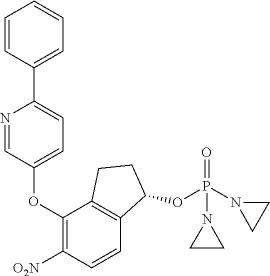 [SMILES: O=[N+]([O-])c1ccc2c(c1Oc1ccc(-c3ccccc3)nc1)CC[C@@H]2OP(=O)(N1CC1)N1CC1]

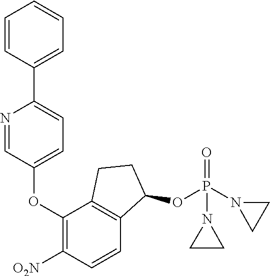 [SMILES: O=[N+]([O-])c1ccc2c(c1Oc1ccc(-c3ccccc3)nc1)CC[C@H]2OP(=O)(N1CC1)N1CC1]

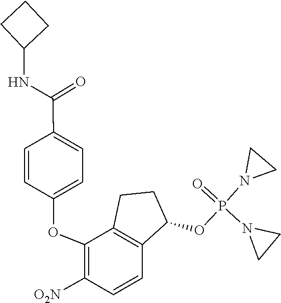 [SMILES: O=C(NC1CCC1)c1ccc(Oc2c([N+](=O)[O-])ccc3c2CC[C@@H]3OP(=O)(N2CC2)N2CC2)cc1]

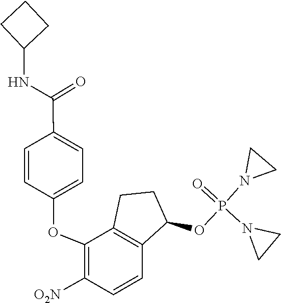 [SMILES: O=C(NC1CCC1)c1ccc(Oc2c([N+](=O)[O-])ccc3c2CC[C@H]3OP(=O)(N2CC2)N2CC2)cc1]

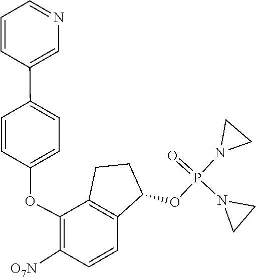

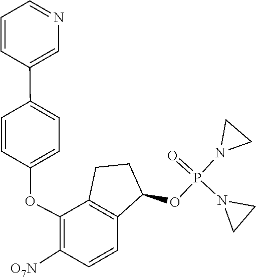

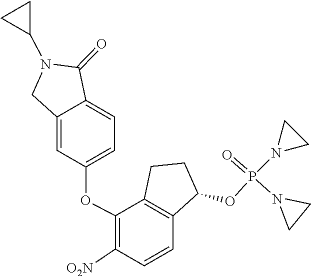 [SMILES: O=C1c2ccc(Oc3c([N+](=O)[O-])ccc4c3CC[C@@H]4OP(=O)(N3CC3)N3CC3)cc2CN1C1CC1]

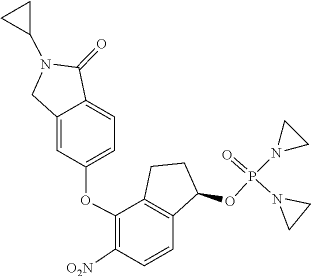 [SMILES: O=C1c2ccc(Oc3c([N+](=O)[O-])ccc4c3CC[C@H]4OP(=O)(N3CC3)N3CC3)cc2CN1C1CC1]

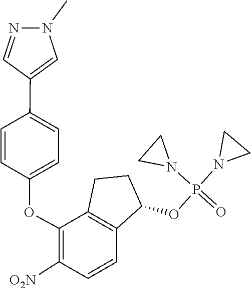 [SMILES: Cn1cc(-c2ccc(Oc3c([N+](=O)[O-])ccc4c3CC[C@@H]4OP(=O)(N3CC3)N3CC3)cc2)cn1]

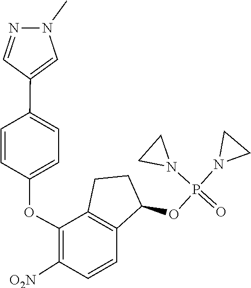 [SMILES: Cn1cc(-c2ccc(Oc3c([N+](=O)[O-])ccc4c3CC[C@H]4OP(=O)(N3CC3)N3CC3)cc2)cn1]

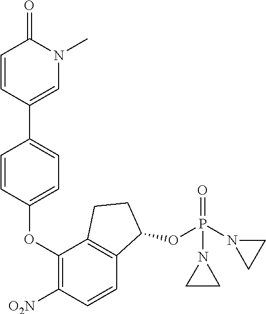 [SMILES: Cn1cc(-c2ccc(Oc3c([N+](=O)[O-])ccc4c3CC[C@@H]4OP(=O)(N3CC3)N3CC3)cc2)ccc1=O]

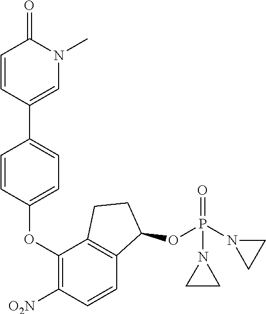 [SMILES: Cn1cc(-c2ccc(Oc3c([N+](=O)[O-])ccc4c3CC[C@H]4OP(=O)(N3CC3)N3CC3)cc2)ccc1=O]

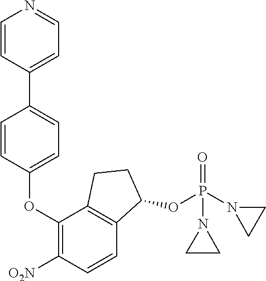 [SMILES: O=[N+]([O-])c1ccc2c(c1Oc1ccc(-c3ccncc3)cc1)CC[C@@H]2OP(=O)(N1CC1)N1CC1]

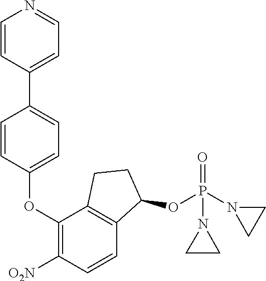 [SMILES: O=[N+]([O-])c1ccc2c(c1Oc1ccc(-c3ccncc3)cc1)CC[C@H]2OP(=O)(N1CC1)N1CC1]

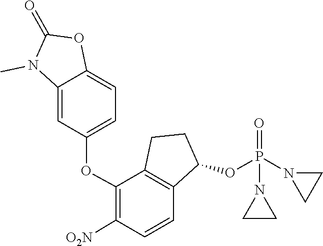 [SMILES: Cn1c(=O)oc2ccc(Oc3c([N+](=O)[O-])ccc4c3CC[C@@H]4OP(=O)(N3CC3)N3CC3)cc21]

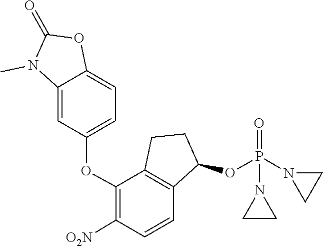 [SMILES: Cn1c(=O)oc2ccc(Oc3c([N+](=O)[O-])ccc4c3CC[C@H]4OP(=O)(N3CC3)N3CC3)cc21]

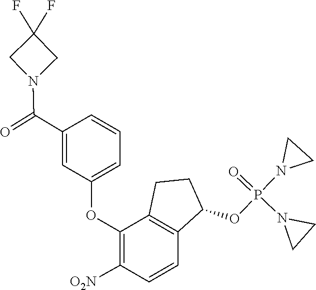 [SMILES: O=C(c1cccc(Oc2c([N+](=O)[O-])ccc3c2CC[C@@H]3OP(=O)(N2CC2)N2CC2)c1)N1CC(F)(F)C1]

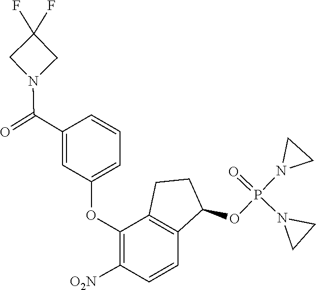 [SMILES: O=C(c1cccc(Oc2c([N+](=O)[O-])ccc3c2CC[C@H]3OP(=O)(N2CC2)N2CC2)c1)N1CC(F)(F)C1]

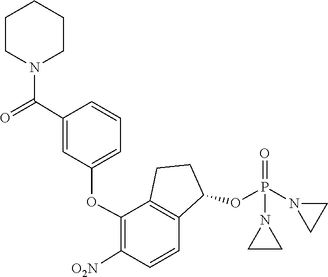 [SMILES: O=C(c1cccc(Oc2c([N+](=O)[O-])ccc3c2CC[C@@H]3OP(=O)(N2CC2)N2CC2)c1)N1CCCCC1]

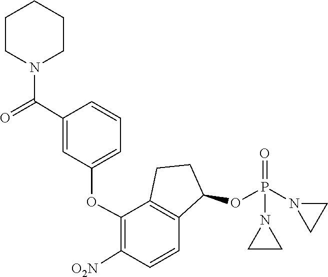 [SMILES: O=C(c1cccc(Oc2c([N+](=O)[O-])ccc3c2CC[C@H]3OP(=O)(N2CC2)N2CC2)c1)N1CCCCC1]

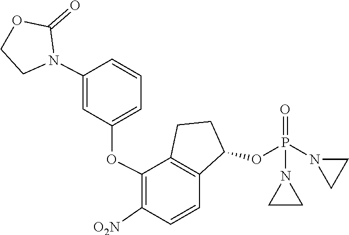 [SMILES: O=C1OCCN1c1cccc(Oc2c([N+](=O)[O-])ccc3c2CC[C@@H]3OP(=O)(N2CC2)N2CC2)c1]

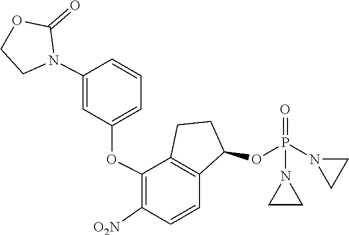 [SMILES: O=C1OCCN1c1cccc(Oc2c([N+](=O)[O-])ccc3c2CC[C@H]3OP(=O)(N2CC2)N2CC2)c1]

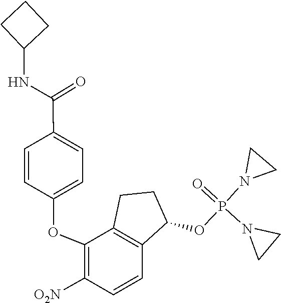 [SMILES: O=C(NC1CCC1)c1ccc(Oc2c([N+](=O)[O-])ccc3c2CC[C@@H]3OP(=O)(N2CC2)N2CC2)cc1]

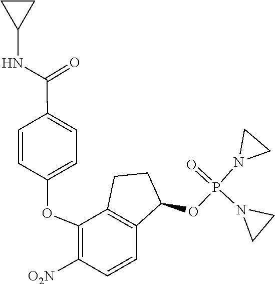 [SMILES: O=C(NC1CC1)c1ccc(Oc2c([N+](=O)[O-])ccc3c2CC[C@H]3OP(=O)(N2CC2)N2CC2)cc1]

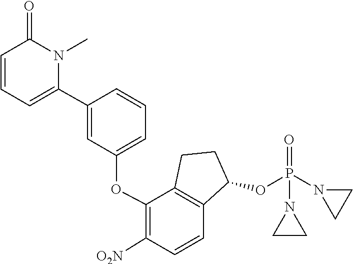 [SMILES: Cn1c(-c2cccc(Oc3c([N+](=O)[O-])ccc4c3CC[C@@H]4OP(=O)(N3CC3)N3CC3)c2)cccc1=O]

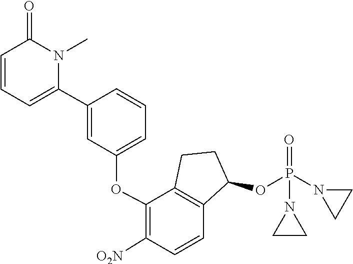 [SMILES: Cn1c(-c2cccc(Oc3c([N+](=O)[O-])ccc4c3CC[C@H]4OP(=O)(N3CC3)N3CC3)c2)cccc1=O]

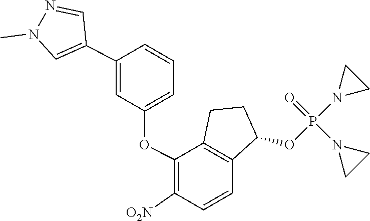 [SMILES: Cn1cc(-c2cccc(Oc3c([N+](=O)[O-])ccc4c3CC[C@@H]4OP(=O)(N3CC3)N3CC3)c2)cn1]

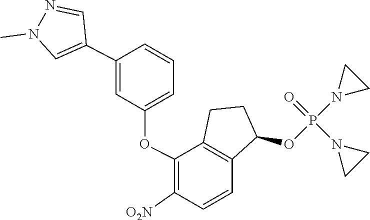 [SMILES: Cn1cc(-c2cccc(Oc3c([N+](=O)[O-])ccc4c3CC[C@H]4OP(=O)(N3CC3)N3CC3)c2)cn1]

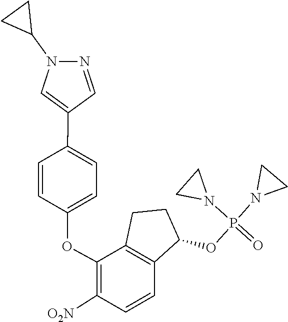 [SMILES: O=[N+]([O-])c1ccc2c(c1Oc1ccc(-c3cnn(C4CC4)c3)cc1)CC[C@@H]2OP(=O)(N1CC1)N1CC1]

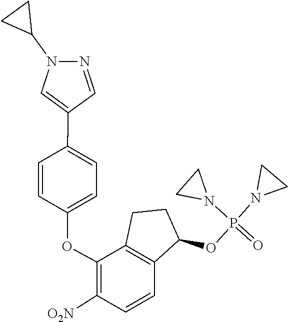 [SMILES: O=[N+]([O-])c1ccc2c(c1Oc1ccc(-c3cnn(C4CC4)c3)cc1)CC[C@H]2OP(=O)(N1CC1)N1CC1]

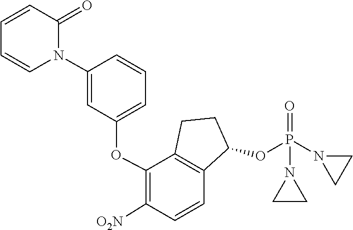 [SMILES: O=c1ccccn1-c1cccc(Oc2c([N+](=O)[O-])ccc3c2CC[C@@H]3OP(=O)(N2CC2)N2CC2)c1]

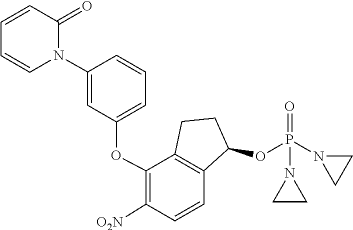 [SMILES: O=c1ccccn1-c1cccc(Oc2c([N+](=O)[O-])ccc3c2CC[C@H]3OP(=O)(N2CC2)N2CC2)c1]

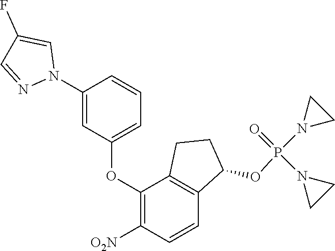 [SMILES: O=[N+]([O-])c1ccc2c(c1Oc1cccc(-n3cc(F)cn3)c1)CC[C@@H]2OP(=O)(N1CC1)N1CC1]

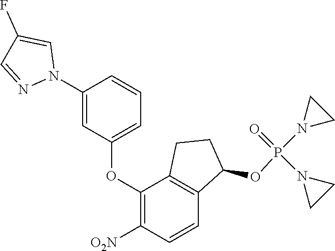 [SMILES: O=[N+]([O-])c1ccc2c(c1Oc1cccc(-n3cc(F)cn3)c1)CC[C@H]2OP(=O)(N1CC1)N1CC1]

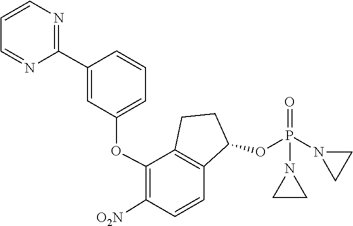 [SMILES: O=[N+]([O-])c1ccc2c(c1Oc1cccc(-c3ncccn3)c1)CC[C@@H]2OP(=O)(N1CC1)N1CC1]

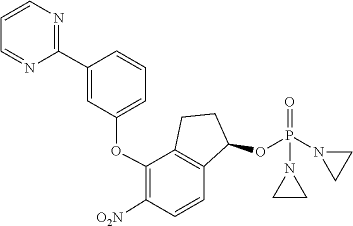 [SMILES: O=[N+]([O-])c1ccc2c(c1Oc1cccc(-c3ncccn3)c1)CC[C@H]2OP(=O)(N1CC1)N1CC1]

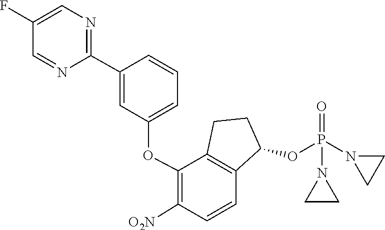 [SMILES: O=[N+]([O-])c1ccc2c(c1Oc1cccc(-c3ncc(F)cn3)c1)CC[C@@H]2OP(=O)(N1CC1)N1CC1]

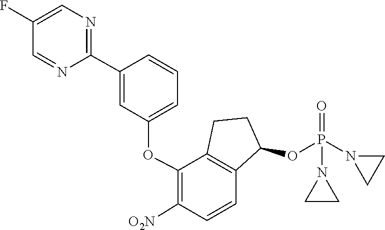 [SMILES: O=[N+]([O-])c1ccc2c(c1Oc1cccc(-c3ncc(F)cn3)c1)CC[C@H]2OP(=O)(N1CC1)N1CC1]

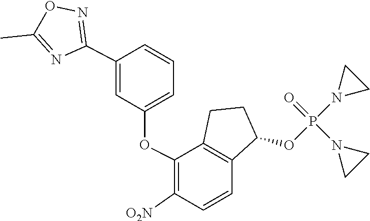 [SMILES: Cc1nc(-c2cccc(Oc3c([N+](=O)[O-])ccc4c3CC[C@@H]4OP(=O)(N3CC3)N3CC3)c2)no1]

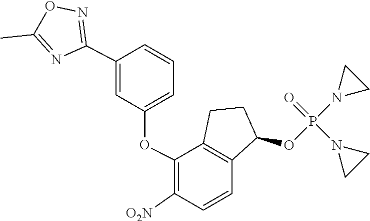 [SMILES: Cc1nc(-c2cccc(Oc3c([N+](=O)[O-])ccc4c3CC[C@H]4OP(=O)(N3CC3)N3CC3)c2)no1]

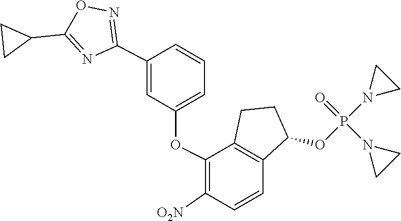 [SMILES: O=[N+]([O-])c1ccc2c(c1Oc1cccc(-c3noc(C4CC4)n3)c1)CC[C@@H]2OP(=O)(N1CC1)N1CC1]

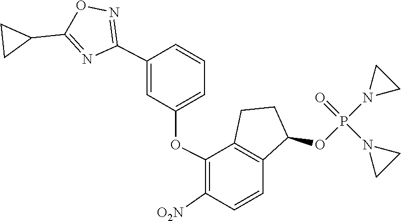 [SMILES: O=[N+]([O-])c1ccc2c(c1Oc1cccc(-c3noc(C4CC4)n3)c1)CC[C@H]2OP(=O)(N1CC1)N1CC1]

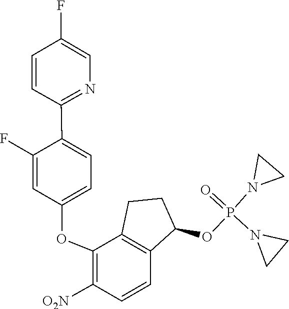 [SMILES: O=[N+]([O-])c1ccc2c(c1Oc1ccc(-c3ccc(F)cn3)c(F)c1)CC[C@H]2OP(=O)(N1CC1)N1CC1]

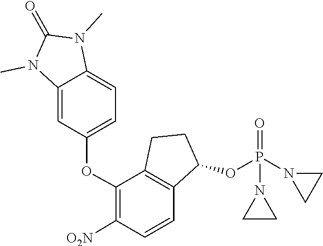 [SMILES: Cn1c(=O)n(C)c2cc(Oc3c([N+](=O)[O-])ccc4c3CC[C@@H]4OP(=O)(N3CC3)N3CC3)ccc21]

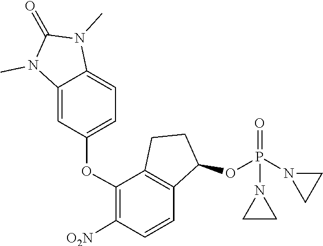 [SMILES: Cn1c(=O)n(C)c2cc(Oc3c([N+](=O)[O-])ccc4c3CC[C@H]4OP(=O)(N3CC3)N3CC3)ccc21]

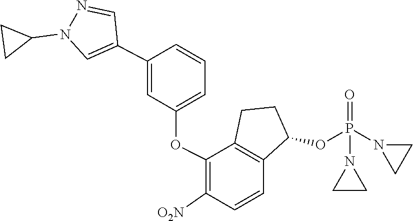 [SMILES: O=[N+]([O-])c1ccc2c(c1Oc1cccc(-c3cnn(C4CC4)c3)c1)CC[C@@H]2OP(=O)(N1CC1)N1CC1]

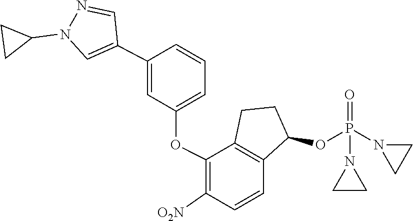 [SMILES: O=[N+]([O-])c1ccc2c(c1Oc1cccc(-c3cnn(C4CC4)c3)c1)CC[C@H]2OP(=O)(N1CC1)N1CC1]

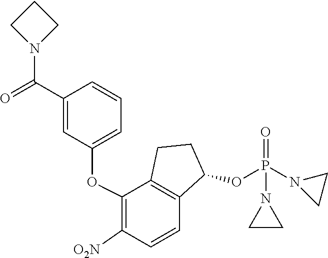 [SMILES: O=C(c1cccc(Oc2c([N+](=O)[O-])ccc3c2CC[C@@H]3OP(=O)(N2CC2)N2CC2)c1)N1CCC1]

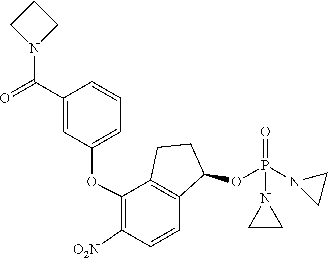 [SMILES: O=C(c1cccc(Oc2c([N+](=O)[O-])ccc3c2CC[C@H]3OP(=O)(N2CC2)N2CC2)c1)N1CCC1]

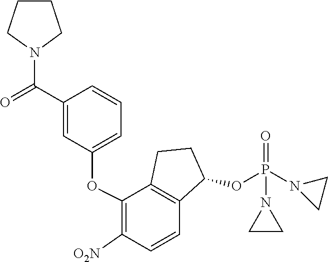 [SMILES: O=C(c1cccc(Oc2c([N+](=O)[O-])ccc3c2CC[C@@H]3OP(=O)(N2CC2)N2CC2)c1)N1CCCC1]

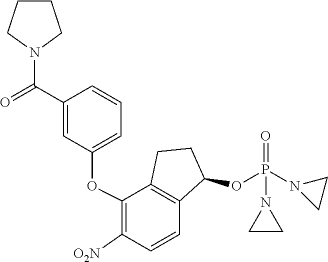 [SMILES: O=C(c1cccc(Oc2c([N+](=O)[O-])ccc3c2CC[C@H]3OP(=O)(N2CC2)N2CC2)c1)N1CCCC1]

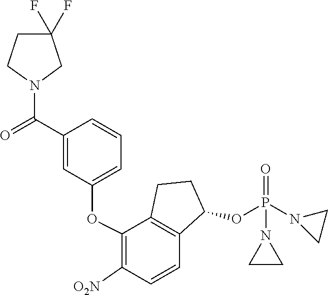 [SMILES: O=C(c1cccc(Oc2c([N+](=O)[O-])ccc3c2CC[C@@H]3OP(=O)(N2CC2)N2CC2)c1)N1CCC(F)(F)C1]

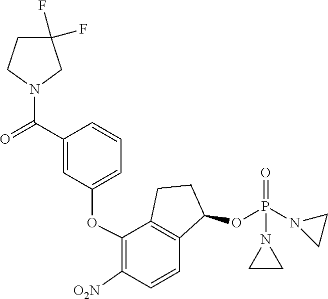 [SMILES: O=C(c1cccc(Oc2c([N+](=O)[O-])ccc3c2CC[C@H]3OP(=O)(N2CC2)N2CC2)c1)N1CCC(F)(F)C1]

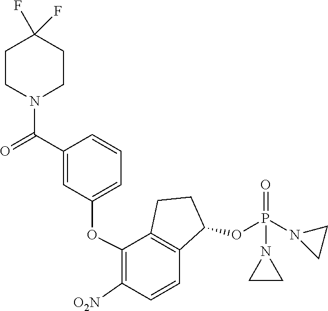 [SMILES: O=C(c1cccc(Oc2c([N+](=O)[O-])ccc3c2CC[C@@H]3OP(=O)(N2CC2)N2CC2)c1)N1CCC(F)(F)CC1]

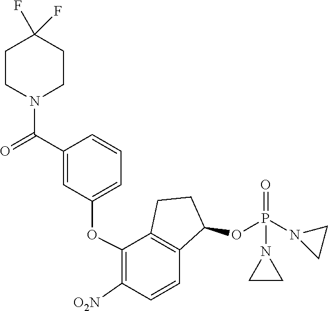 [SMILES: O=C(c1cccc(Oc2c([N+](=O)[O-])ccc3c2CC[C@H]3OP(=O)(N2CC2)N2CC2)c1)N1CCC(F)(F)CC1]

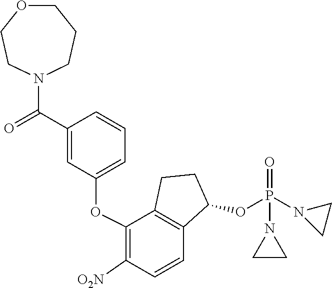 [SMILES: O=C(c1cccc(Oc2c([N+](=O)[O-])ccc3c2CC[C@@H]3OP(=O)(N2CC2)N2CC2)c1)N1CCCOCC1]

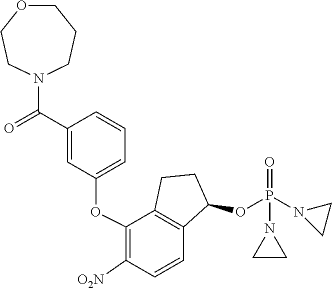 [SMILES: O=C(c1cccc(Oc2c([N+](=O)[O-])ccc3c2CC[C@H]3OP(=O)(N2CC2)N2CC2)c1)N1CCCOCC1]

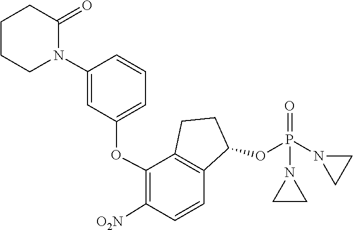 [SMILES: O=C1CCCCN1c1cccc(Oc2c([N+](=O)[O-])ccc3c2CC[C@@H]3OP(=O)(N2CC2)N2CC2)c1]

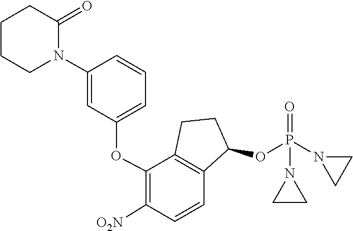 [SMILES: O=C1CCCCN1c1cccc(Oc2c([N+](=O)[O-])ccc3c2CC[C@H]3OP(=O)(N2CC2)N2CC2)c1]

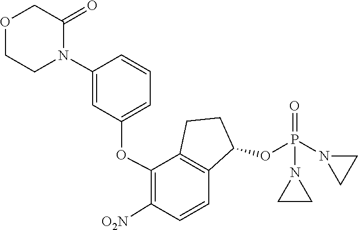 [SMILES: O=C1COCCN1c1cccc(Oc2c([N+](=O)[O-])ccc3c2CC[C@@H]3OP(=O)(N2CC2)N2CC2)c1]

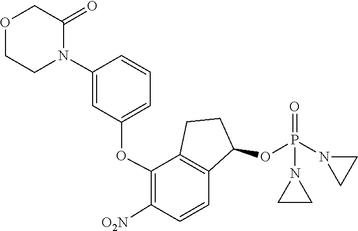 [SMILES: O=C1COCCN1c1cccc(Oc2c([N+](=O)[O-])ccc3c2CC[C@H]3OP(=O)(N2CC2)N2CC2)c1]

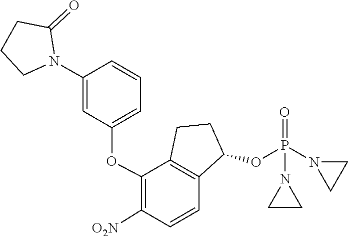 [SMILES: O=C1CCCN1c1cccc(Oc2c([N+](=O)[O-])ccc3c2CC[C@@H]3OP(=O)(N2CC2)N2CC2)c1]

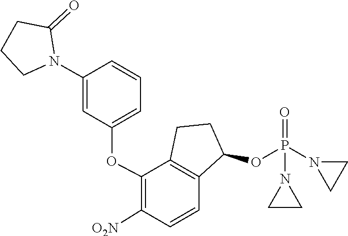 [SMILES: O=C1CCCN1c1cccc(Oc2c([N+](=O)[O-])ccc3c2CC[C@H]3OP(=O)(N2CC2)N2CC2)c1]

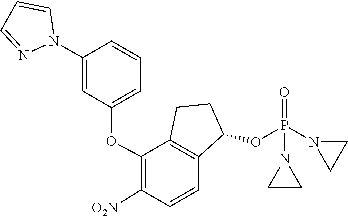 [SMILES: O=[N+]([O-])c1ccc2c(c1Oc1cccc(-n3cccn3)c1)CC[C@@H]2OP(=O)(N1CC1)N1CC1]

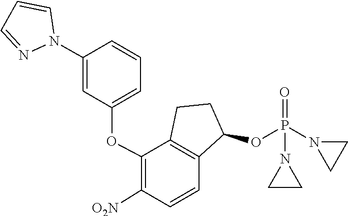 [SMILES: O=[N+]([O-])c1ccc2c(c1Oc1cccc(-n3cccn3)c1)CC[C@H]2OP(=O)(N1CC1)N1CC1]

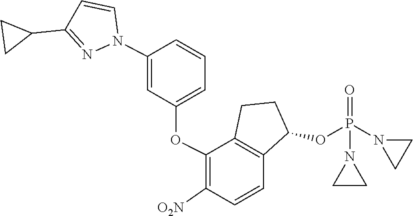 [SMILES: O=[N+]([O-])c1ccc2c(c1Oc1cccc(-n3ccc(C4CC4)n3)c1)CC[C@@H]2OP(=O)(N1CC1)N1CC1]

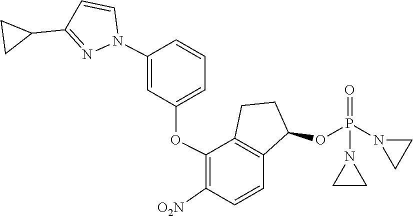 [SMILES: O=[N+]([O-])c1ccc2c(c1Oc1cccc(-n3ccc(C4CC4)n3)c1)CC[C@H]2OP(=O)(N1CC1)N1CC1]

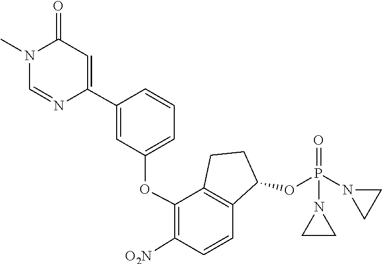 [SMILES: Cn1cnc(-c2cccc(Oc3c([N+](=O)[O-])ccc4c3CC[C@@H]4OP(=O)(N3CC3)N3CC3)c2)cc1=O]

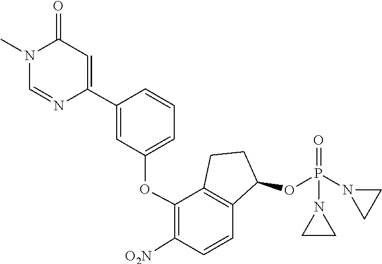 [SMILES: Cn1cnc(-c2cccc(Oc3c([N+](=O)[O-])ccc4c3CC[C@H]4OP(=O)(N3CC3)N3CC3)c2)cc1=O]

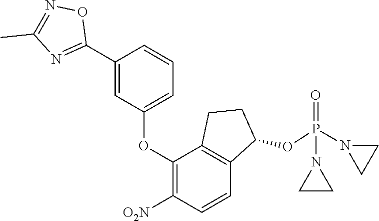 [SMILES: Cc1noc(-c2cccc(Oc3c([N+](=O)[O-])ccc4c3CC[C@@H]4OP(=O)(N3CC3)N3CC3)c2)n1]

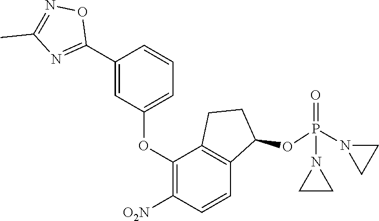 [SMILES: Cc1noc(-c2cccc(Oc3c([N+](=O)[O-])ccc4c3CC[C@H]4OP(=O)(N3CC3)N3CC3)c2)n1]

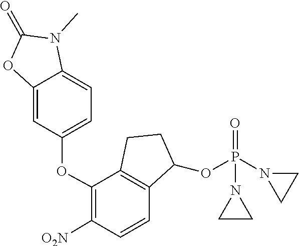 [SMILES: Cn1c(=O)oc2cc(Oc3c([N+](=O)[O-])ccc4c3CCC4OP(=O)(N3CC3)N3CC3)ccc21]

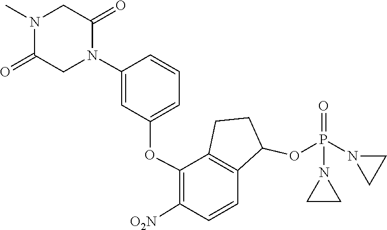 [SMILES: CN1CC(=O)N(c2cccc(Oc3c([N+](=O)[O-])ccc4c3CCC4OP(=O)(N3CC3)N3CC3)c2)CC1=O]

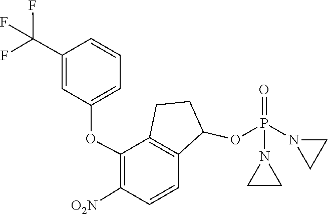 [SMILES: O=[N+]([O-])c1ccc2c(c1Oc1cccc(C(F)(F)F)c1)CCC2OP(=O)(N1CC1)N1CC1]

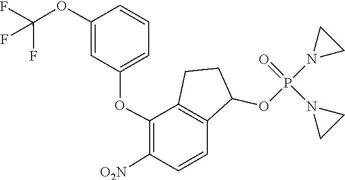 [SMILES: O=[N+]([O-])c1ccc2c(c1Oc1cccc(OC(F)(F)F)c1)CCC2OP(=O)(N1CC1)N1CC1]

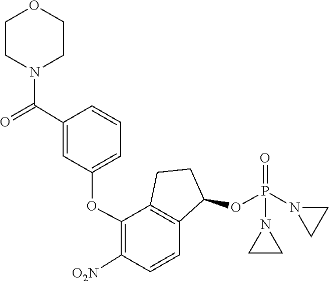 [SMILES: O=C(c1cccc(Oc2c([N+](=O)[O-])ccc3c2CC[C@H]3OP(=O)(N2CC2)N2CC2)c1)N1CCOCC1]

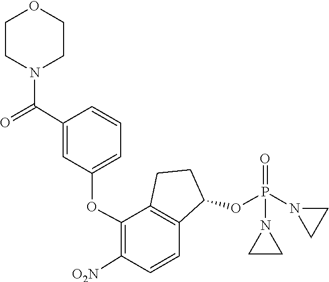 [SMILES: O=C(c1cccc(Oc2c([N+](=O)[O-])ccc3c2CC[C@@H]3OP(=O)(N2CC2)N2CC2)c1)N1CCOCC1]

TABLE A6
List of compounds

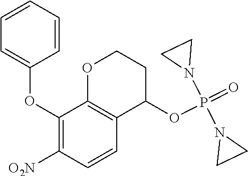 [SMILES: O=[N+]([O-])c1ccc2c(c1Oc1ccccc1)OCCC2OP(=O)(N1CC1)N1CC1]

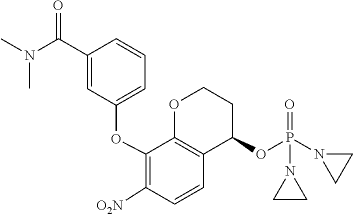 [SMILES: CN(C)C(=O)c1cccc(Oc2c([N+](=O)[O-])ccc3c2OCC[C@H]3OP(=O)(N2CC2)N2CC2)c1]

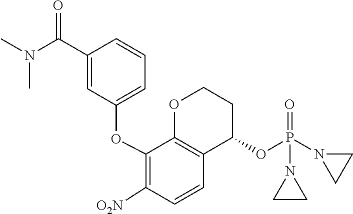 [SMILES: CN(C)C(=O)c1cccc(Oc2c([N+](=O)[O-])ccc3c2OCC[C@@H]3OP(=O)(N2CC2)N2CC2)c1]

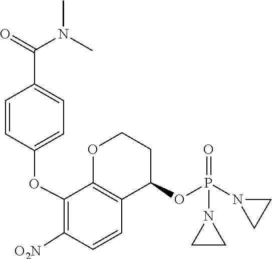 [SMILES: CN(C)C(=O)c1ccc(Oc2c([N+](=O)[O-])ccc3c2OCC[C@H]3OP(=O)(N2CC2)N2CC2)cc1]

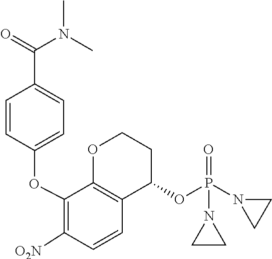 [SMILES: CN(C)C(=O)c1ccc(Oc2c([N+](=O)[O-])ccc3c2OCC[C@@H]3OP(=O)(N2CC2)N2CC2)cc1]

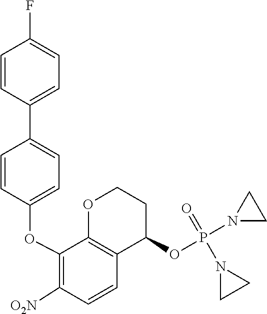 [SMILES: O=[N+]([O-])c1ccc2c(c1Oc1ccc(-c3ccc(F)cc3)cc1)OCC[C@H]2OP(=O)(N1CC1)N1CC1]

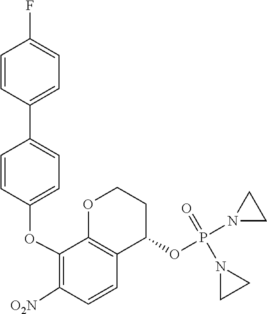 [SMILES: O=[N+]([O-])c1ccc2c(c1Oc1ccc(-c3ccc(F)cc3)cc1)OCC[C@@H]2OP(=O)(N1CC1)N1CC1]

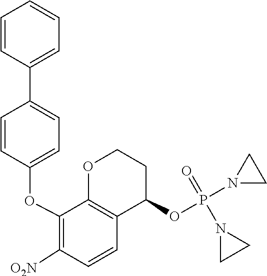 [SMILES: O=[N+]([O-])c1ccc2c(c1Oc1ccc(-c3ccccc3)cc1)OCC[C@H]2OP(=O)(N1CC1)N1CC1]

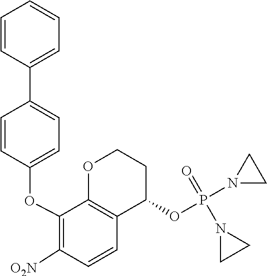 [SMILES: O=[N+]([O-])c1ccc2c(c1Oc1ccc(-c3ccccc3)cc1)OCC[C@@H]2OP(=O)(N1CC1)N1CC1]

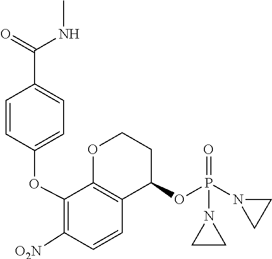 [SMILES: CNC(=O)c1ccc(Oc2c([N+](=O)[O-])ccc3c2OCC[C@H]3OP(=O)(N2CC2)N2CC2)cc1]

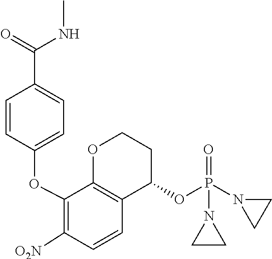 [SMILES: CNC(=O)c1ccc(Oc2c([N+](=O)[O-])ccc3c2OCC[C@@H]3OP(=O)(N2CC2)N2CC2)cc1]

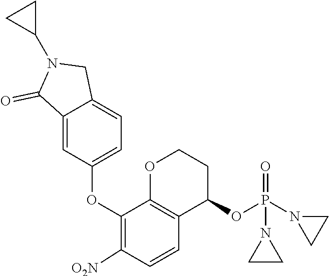 [SMILES: O=C1c2cc(Oc3c([N+](=O)[O-])ccc4c3OCC[C@H]4OP(=O)(N3CC3)N3CC3)ccc2CN1C1CC1]

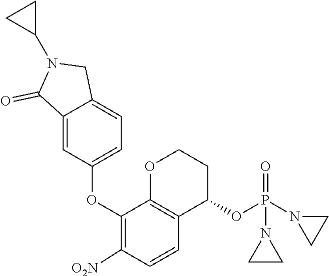 [SMILES: O=C1c2cc(Oc3c([N+](=O)[O-])ccc4c3OCC[C@@H]4OP(=O)(N3CC3)N3CC3)ccc2CN1C1CC1]

TABLE A7
List of compounds

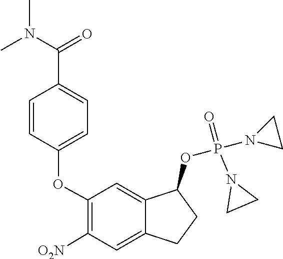 [SMILES: CN(C)C(=O)c1ccc(Oc2cc3c(cc2[N+](=O)[O-])CC[C@@H]3OP(=O)(N2CC2)N2CC2)cc1]

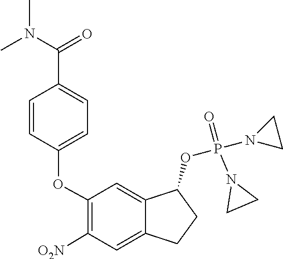 [SMILES: CN(C)C(=O)c1ccc(Oc2cc3c(cc2[N+](=O)[O-])CC[C@H]3OP(=O)(N2CC2)N2CC2)cc1]

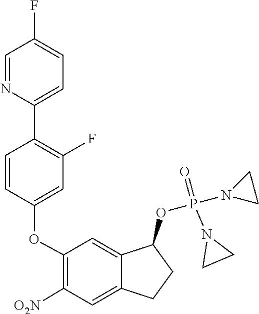 [SMILES: O=[N+]([O-])c1cc2c(cc1Oc1ccc(-c3ccc(F)cn3)c(F)c1)[C@@H](OP(=O)(N1CC1)N1CC1)CC2]

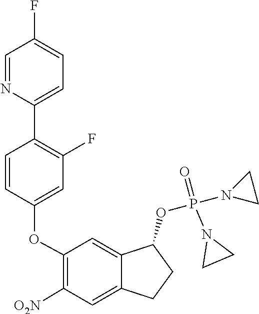 [SMILES: O=[N+]([O-])c1cc2c(cc1Oc1ccc(-c3ccc(F)cn3)c(F)c1)[C@H](OP(=O)(N1CC1)N1CC1)CC2]

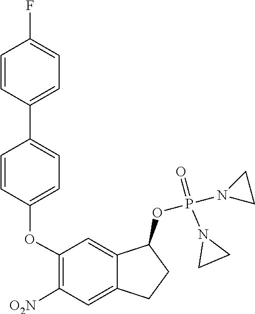 [SMILES: O=[N+]([O-])c1cc2c(cc1Oc1ccc(-c3ccc(F)cc3)cc1)[C@@H](OP(=O)(N1CC1)N1CC1)CC2]

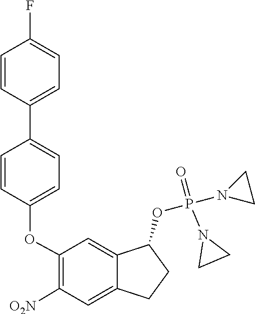 [SMILES: O=[N+]([O-])c1cc2c(cc1Oc1ccc(-c3ccc(F)cc3)cc1)[C@H](OP(=O)(N1CC1)N1CC1)CC2]

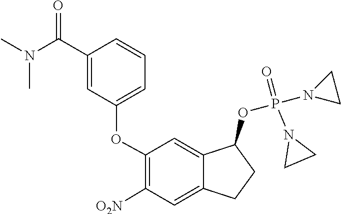 [SMILES: CN(C)C(=O)c1cccc(Oc2cc3c(cc2[N+](=O)[O-])CC[C@@H]3OP(=O)(N2CC2)N2CC2)c1]

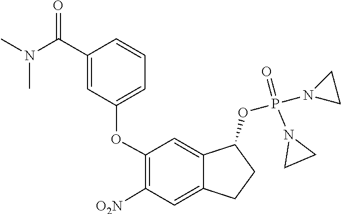 [SMILES: CN(C)C(=O)c1cccc(Oc2cc3c(cc2[N+](=O)[O-])CC[C@H]3OP(=O)(N2CC2)N2CC2)c1]

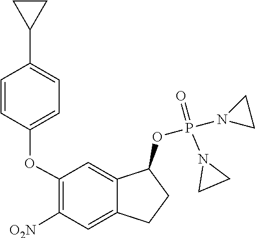 [SMILES: O=[N+]([O-])c1cc2c(cc1Oc1ccc(C3CC3)cc1)[C@@H](OP(=O)(N1CC1)N1CC1)CC2]

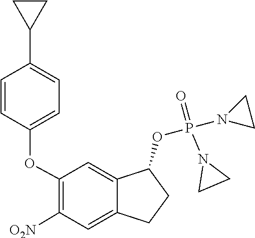 [SMILES: O=[N+]([O-])c1cc2c(cc1Oc1ccc(C3CC3)cc1)[C@H](OP(=O)(N1CC1)N1CC1)CC2]

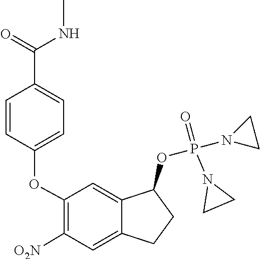 [SMILES: CNC(=O)c1ccc(Oc2cc3c(cc2[N+](=O)[O-])CC[C@@H]3OP(=O)(N2CC2)N2CC2)cc1]

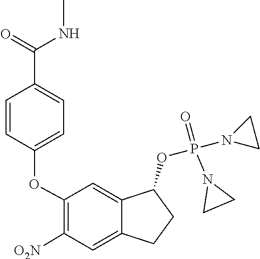 [SMILES: CNC(=O)c1ccc(Oc2cc3c(cc2[N+](=O)[O-])CC[C@H]3OP(=O)(N2CC2)N2CC2)cc1]

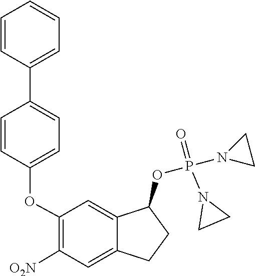 [SMILES: O=[N+]([O-])c1cc2c(cc1Oc1ccc(-c3ccccc3)cc1)[C@@H](OP(=O)(N1CC1)N1CC1)CC2]

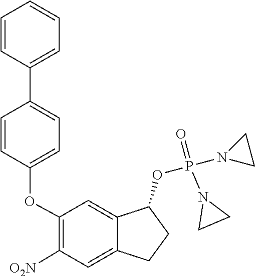 [SMILES: O=[N+]([O-])c1cc2c(cc1Oc1ccc(-c3ccccc3)cc1)[C@H](OP(=O)(N1CC1)N1CC1)CC2]

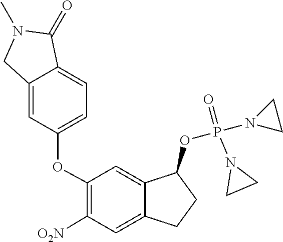 [SMILES: CN1Cc2cc(Oc3cc4c(cc3[N+](=O)[O-])CC[C@@H]4OP(=O)(N3CC3)N3CC3)ccc2C1=O]

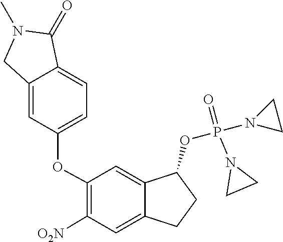 [SMILES: CN1Cc2cc(Oc3cc4c(cc3[N+](=O)[O-])CC[C@H]4OP(=O)(N3CC3)N3CC3)ccc2C1=O]

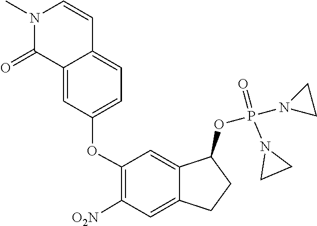 [SMILES: Cn1ccc2ccc(Oc3cc4c(cc3[N+](=O)[O-])CC[C@@H]4OP(=O)(N3CC3)N3CC3)cc2c1=O]

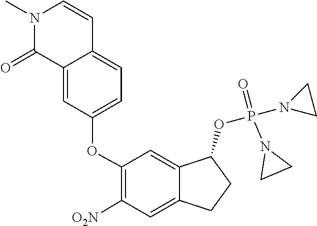 [SMILES: Cn1ccc2ccc(Oc3cc4c(cc3[N+](=O)[O-])CC[C@H]4OP(=O)(N3CC3)N3CC3)cc2c1=O]

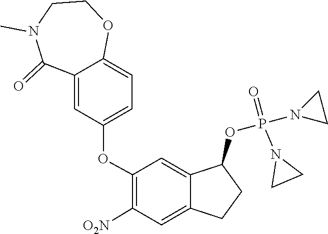 [SMILES: CN1CCOc2ccc(Oc3cc4c(cc3[N+](=O)[O-])CC[C@@H]4OP(=O)(N3CC3)N3CC3)cc2C1=O]

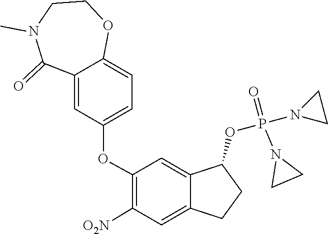 [SMILES: CN1CCOc2ccc(Oc3cc4c(cc3[N+](=O)[O-])CC[C@H]4OP(=O)(N3CC3)N3CC3)cc2C1=O]

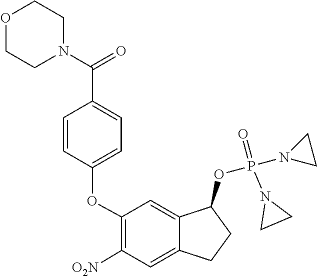 [SMILES: O=C(c1ccc(Oc2cc3c(cc2[N+](=O)[O-])CC[C@@H]3OP(=O)(N2CC2)N2CC2)cc1)N1CCOCC1]

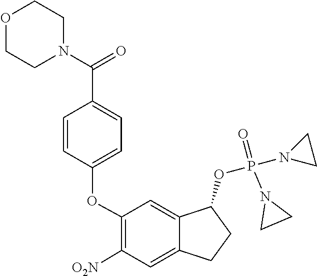 [SMILES: O=C(c1ccc(Oc2cc3c(cc2[N+](=O)[O-])CC[C@H]3OP(=O)(N2CC2)N2CC2)cc1)N1CCOCC1]

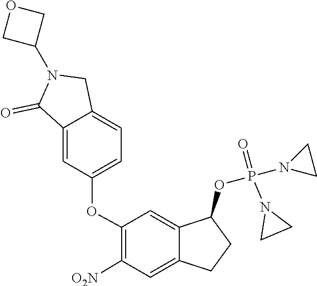 [SMILES: O=C1c2cc(Oc3cc4c(cc3[N+](=O)[O-])CC[C@@H]4OP(=O)(N3CC3)N3CC3)ccc2CN1C1COC1]

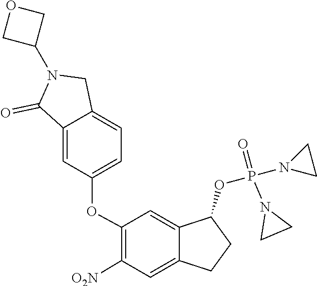 [SMILES: O=C1c2cc(Oc3cc4c(cc3[N+](=O)[O-])CC[C@H]4OP(=O)(N3CC3)N3CC3)ccc2CN1C1COC1]

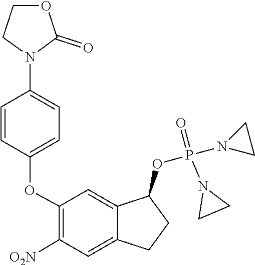 [SMILES: O=C1OCCN1c1ccc(Oc2cc3c(cc2[N+](=O)[O-])CC[C@@H]3OP(=O)(N2CC2)N2CC2)cc1]

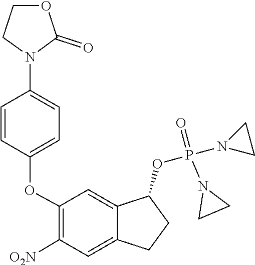 [SMILES: O=C1OCCN1c1ccc(Oc2cc3c(cc2[N+](=O)[O-])CC[C@H]3OP(=O)(N2CC2)N2CC2)cc1]

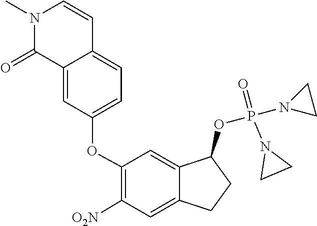 [SMILES: Cn1ccc2ccc(Oc3cc4c(cc3[N+](=O)[O-])CC[C@@H]4OP(=O)(N3CC3)N3CC3)cc2c1=O]

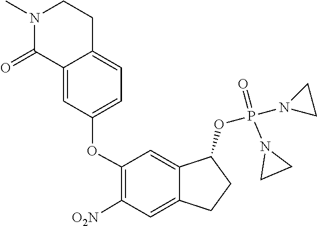 [SMILES: CN1CCc2ccc(Oc3cc4c(cc3[N+](=O)[O-])CC[C@H]4OP(=O)(N3CC3)N3CC3)cc2C1=O]

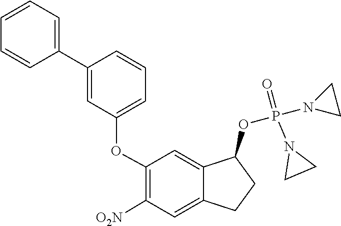 [SMILES: O=[N+]([O-])c1cc2c(cc1Oc1cccc(-c3ccccc3)c1)[C@@H](OP(=O)(N1CC1)N1CC1)CC2]

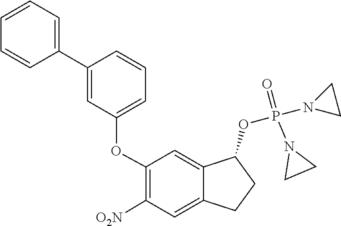 [SMILES: O=[N+]([O-])c1cc2c(cc1Oc1cccc(-c3ccccc3)c1)[C@H](OP(=O)(N1CC1)N1CC1)CC2]

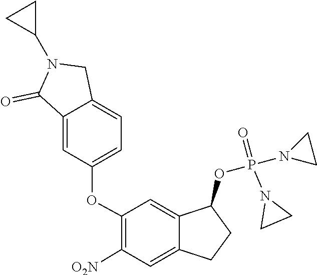 [SMILES: O=C1c2cc(Oc3cc4c(cc3[N+](=O)[O-])CC[C@@H]4OP(=O)(N3CC3)N3CC3)ccc2CN1C1CC1]

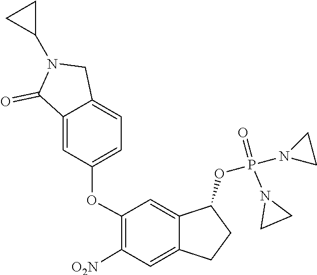 [SMILES: O=C1c2cc(Oc3cc4c(cc3[N+](=O)[O-])CC[C@H]4OP(=O)(N3CC3)N3CC3)ccc2CN1C1CC1]

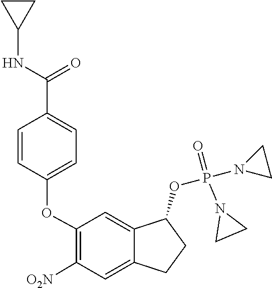 [SMILES: O=C(NC1CC1)c1ccc(Oc2cc3c(cc2[N+](=O)[O-])CC[C@H]3OP(=O)(N2CC2)N2CC2)cc1]

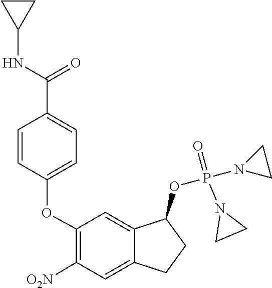 [SMILES: O=C(NC1CC1)c1ccc(Oc2cc3c(cc2[N+](=O)[O-])CC[C@@H]3OP(=O)(N2CC2)N2CC2)cc1]

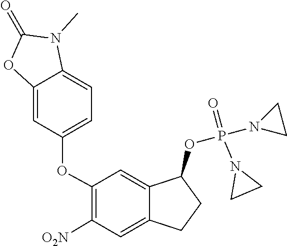 [SMILES: Cn1c(=O)oc2cc(Oc3cc4c(cc3[N+](=O)[O-])CC[C@@H]4OP(=O)(N3CC3)N3CC3)ccc21]

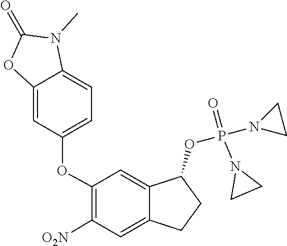 [SMILES: Cn1c(=O)oc2cc(Oc3cc4c(cc3[N+](=O)[O-])CC[C@H]4OP(=O)(N3CC3)N3CC3)ccc21]

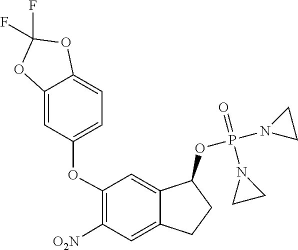 [SMILES: O=[N+]([O-])c1cc2c(cc1Oc1ccc3c(c1)OC(F)(F)O3)[C@@H](OP(=O)(N1CC1)N1CC1)CC2]

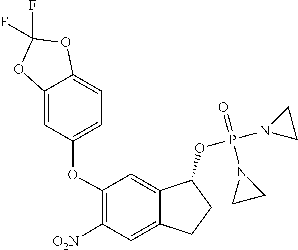 [SMILES: O=[N+]([O-])c1cc2c(cc1Oc1ccc3c(c1)OC(F)(F)O3)[C@H](OP(=O)(N1CC1)N1CC1)CC2]

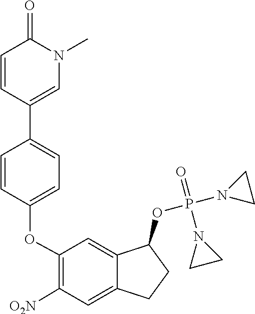 [SMILES: Cn1cc(-c2ccc(Oc3cc4c(cc3[N+](=O)[O-])CC[C@@H]4OP(=O)(N3CC3)N3CC3)cc2)ccc1=O]

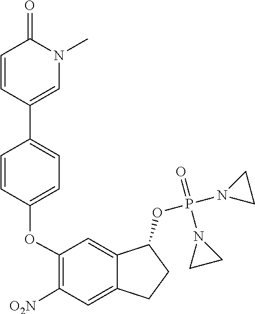 [SMILES: Cn1cc(-c2ccc(Oc3cc4c(cc3[N+](=O)[O-])CC[C@H]4OP(=O)(N3CC3)N3CC3)cc2)ccc1=O]

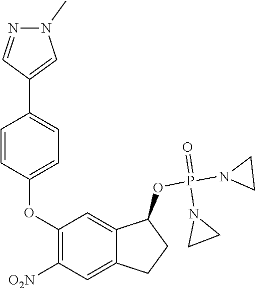 [SMILES: Cn1cc(-c2ccc(Oc3cc4c(cc3[N+](=O)[O-])CC[C@@H]4OP(=O)(N3CC3)N3CC3)cc2)cn1]

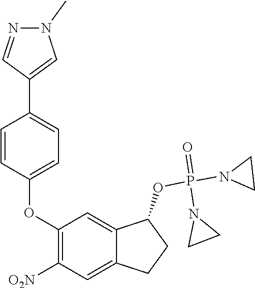 [SMILES: Cn1cc(-c2ccc(Oc3cc4c(cc3[N+](=O)[O-])CC[C@H]4OP(=O)(N3CC3)N3CC3)cc2)cn1]

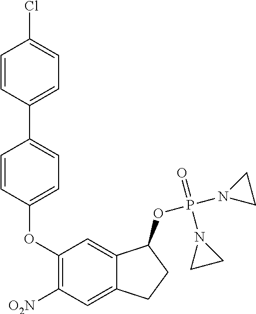 [SMILES: O=[N+]([O-])c1cc2c(cc1Oc1ccc(-c3ccc(Cl)cc3)cc1)[C@@H](OP(=O)(N1CC1)N1CC1)CC2]

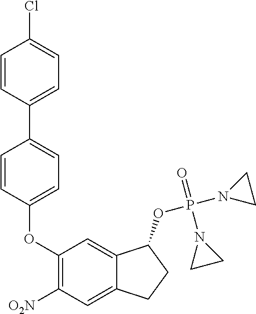 [SMILES: O=[N+]([O-])c1cc2c(cc1Oc1ccc(-c3ccc(Cl)cc3)cc1)[C@H](OP(=O)(N1CC1)N1CC1)CC2]

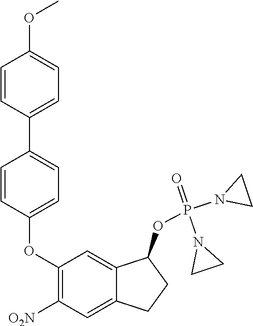 [SMILES: COc1ccc(-c2ccc(Oc3cc4c(cc3[N+](=O)[O-])CC[C@@H]4OP(=O)(N3CC3)N3CC3)cc2)cc1]

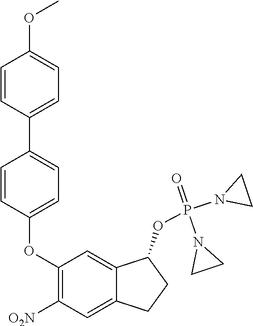 [SMILES: COc1ccc(-c2ccc(Oc3cc4c(cc3[N+](=O)[O-])CC[C@H]4OP(=O)(N3CC3)N3CC3)cc2)cc1]

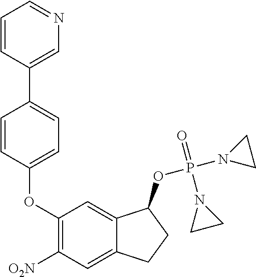 [SMILES: O=[N+]([O-])c1cc2c(cc1Oc1ccc(-c3cccnc3)cc1)[C@@H](OP(=O)(N1CC1)N1CC1)CC2]

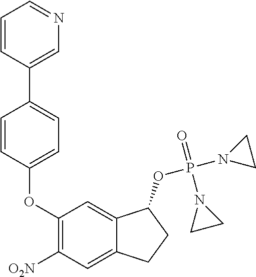 [SMILES: O=[N+]([O-])c1cc2c(cc1Oc1ccc(-c3cccnc3)cc1)[C@H](OP(=O)(N1CC1)N1CC1)CC2]

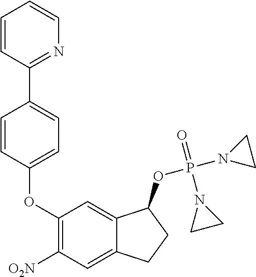 [SMILES: O=[N+]([O-])c1cc2c(cc1Oc1ccc(-c3ccccn3)cc1)[C@@H](OP(=O)(N1CC1)N1CC1)CC2]

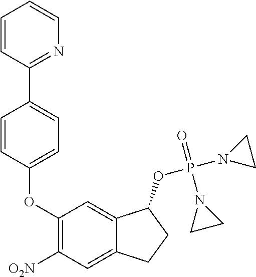 [SMILES: O=[N+]([O-])c1cc2c(cc1Oc1ccc(-c3ccccn3)cc1)[C@H](OP(=O)(N1CC1)N1CC1)CC2]

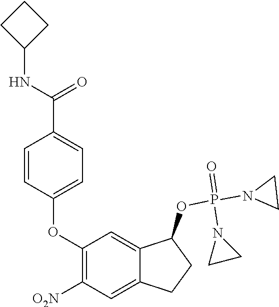 [SMILES: O=C(NC1CCC1)c1ccc(Oc2cc3c(cc2[N+](=O)[O-])CC[C@@H]3OP(=O)(N2CC2)N2CC2)cc1]

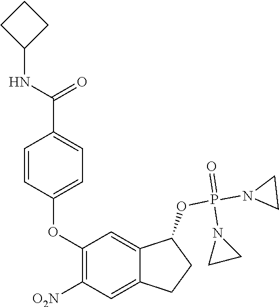 [SMILES: O=C(NC1CCC1)c1ccc(Oc2cc3c(cc2[N+](=O)[O-])CC[C@H]3OP(=O)(N2CC2)N2CC2)cc1]

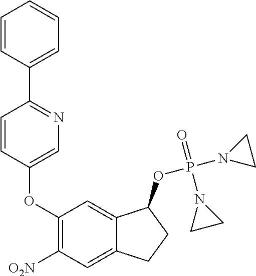 [SMILES: O=[N+]([O-])c1cc2c(cc1Oc1ccc(-c3ccccc3)nc1)[C@@H](OP(=O)(N1CC1)N1CC1)CC2]

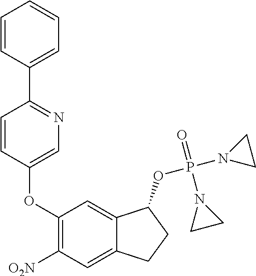 [SMILES: O=[N+]([O-])c1cc2c(cc1Oc1ccc(-c3ccccc3)nc1)[C@H](OP(=O)(N1CC1)N1CC1)CC2]

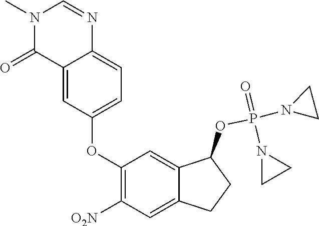 [SMILES: Cn1cnc2ccc(Oc3cc4c(cc3[N+](=O)[O-])CC[C@@H]4OP(=O)(N3CC3)N3CC3)cc2c1=O]

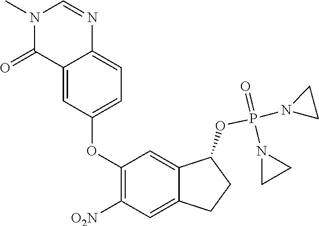 [SMILES: Cn1cnc2ccc(Oc3cc4c(cc3[N+](=O)[O-])CC[C@H]4OP(=O)(N3CC3)N3CC3)cc2c1=O]

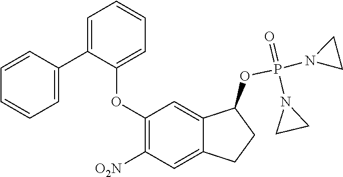 [SMILES: O=[N+]([O-])c1cc2c(cc1Oc1ccccc1-c1ccccc1)[C@@H](OP(=O)(N1CC1)N1CC1)CC2]

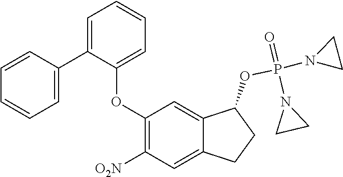 [SMILES: O=[N+]([O-])c1cc2c(cc1Oc1ccccc1-c1ccccc1)[C@H](OP(=O)(N1CC1)N1CC1)CC2]

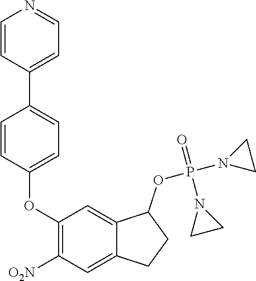 [SMILES: O=[N+]([O-])c1cc2c(cc1Oc1ccc(-c3ccncc3)cc1)C(OP(=O)(N1CC1)N1CC1)CC2]

TABLE A8
List of compounds

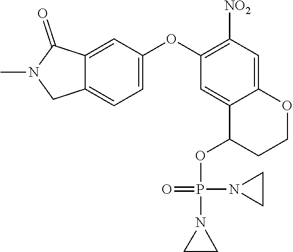 [SMILES: CN1Cc2ccc(Oc3cc4c(cc3[N+](=O)[O-])OCCC4OP(=O)(N3CC3)N3CC3)cc2C1=O]

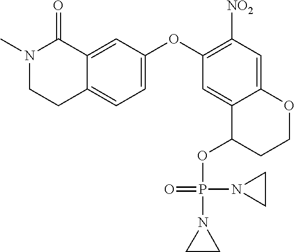 [SMILES: CN1CCc2ccc(Oc3cc4c(cc3[N+](=O)[O-])OCCC4OP(=O)(N3CC3)N3CC3)cc2C1=O]

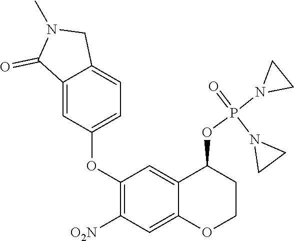 [SMILES: CN1Cc2ccc(Oc3cc4c(cc3[N+](=O)[O-])OCC[C@@H]4OP(=O)(N3CC3)N3CC3)cc2C1=O]

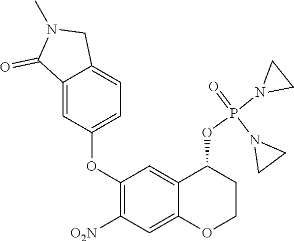 [SMILES: CN1Cc2ccc(Oc3cc4c(cc3[N+](=O)[O-])OCC[C@H]4OP(=O)(N3CC3)N3CC3)cc2C1=O]

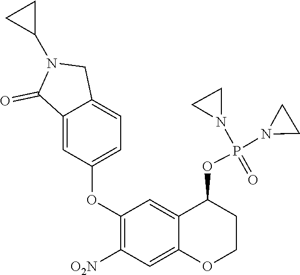 [SMILES: O=C1c2cc(Oc3cc4c(cc3[N+](=O)[O-])OCC[C@@H]4OP(=O)(N3CC3)N3CC3)ccc2CN1C1CC1]

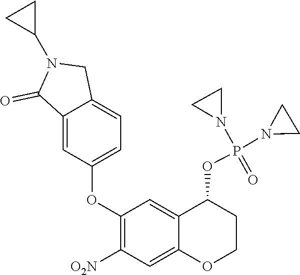 [SMILES: O=C1c2cc(Oc3cc4c(cc3[N+](=O)[O-])OCC[C@H]4OP(=O)(N3CC3)N3CC3)ccc2CN1C1CC1]

TABLE A9
List of compounds

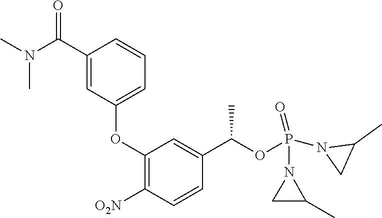

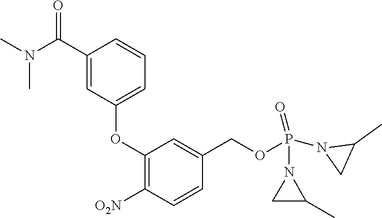

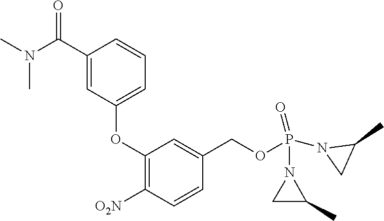

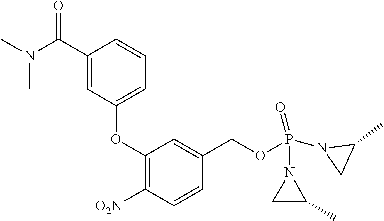

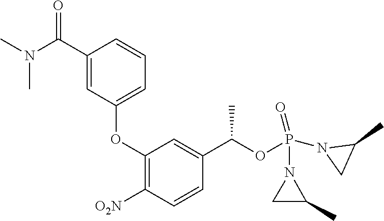

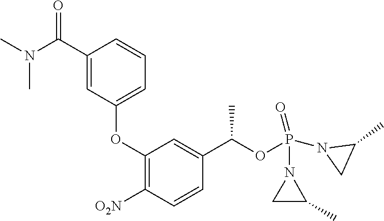

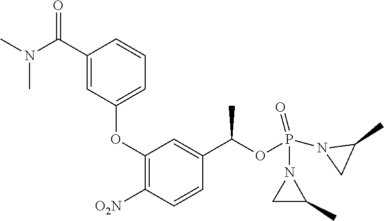

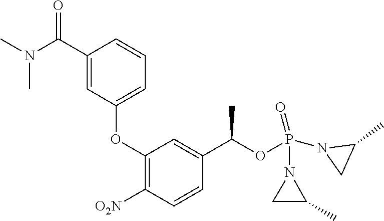

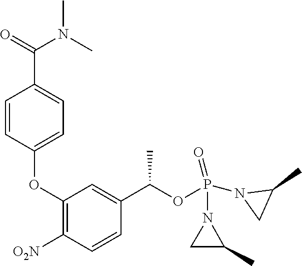 [SMILES: C[C@H](OP(=O)(N1C[C@@H]1C)N1C[C@@H]1C)c1ccc([N+](=O)[O-])c(Oc2ccc(C(=O)N(C)C)cc2)c1]

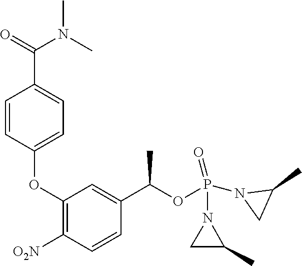 [SMILES: C[C@@H](OP(=O)(N1C[C@@H]1C)N1C[C@@H]1C)c1ccc([N+](=O)[O-])c(Oc2ccc(C(=O)N(C)C)cc2)c1]

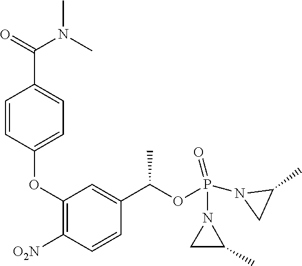 [SMILES: C[C@H](OP(=O)(N1C[C@H]1C)N1C[C@H]1C)c1ccc([N+](=O)[O-])c(Oc2ccc(C(=O)N(C)C)cc2)c1]

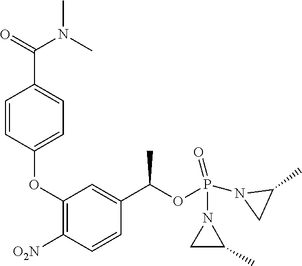 [SMILES: C[C@@H]1CN1P(=O)(O[C@H](C)c1ccc([N+](=O)[O-])c(Oc2ccc(C(=O)N(C)C)cc2)c1)N1C[C@H]1C]

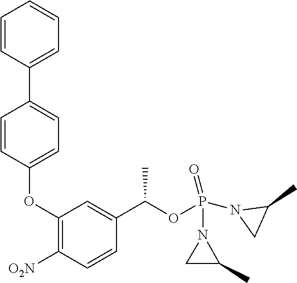 [SMILES: C[C@H](OP(=O)(N1C[C@@H]1C)N1C[C@@H]1C)c1ccc([N+](=O)[O-])c(Oc2ccc(-c3ccccc3)cc2)c1]

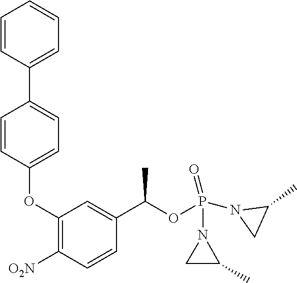 [SMILES: C[C@@H]1CN1P(=O)(O[C@H](C)c1ccc([N+](=O)[O-])c(Oc2ccc(-c3ccccc3)cc2)c1)N1C[C@H]1C]

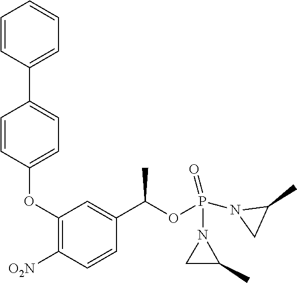 [SMILES: C[C@@H](OP(=O)(N1C[C@@H]1C)N1C[C@@H]1C)c1ccc([N+](=O)[O-])c(Oc2ccc(-c3ccccc3)cc2)c1]

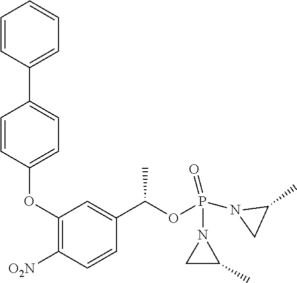 [SMILES: C[C@H](OP(=O)(N1C[C@H]1C)N1C[C@H]1C)c1ccc([N+](=O)[O-])c(Oc2ccc(-c3ccccc3)cc2)c1]

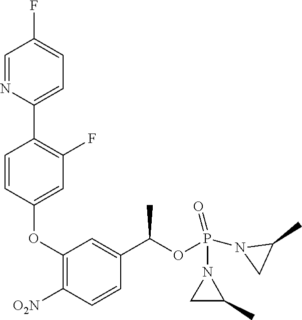 [SMILES: C[C@@H](OP(=O)(N1C[C@@H]1C)N1C[C@@H]1C)c1ccc([N+](=O)[O-])c(Oc2ccc(-c3ccc(F)cn3)c(F)c2)c1]

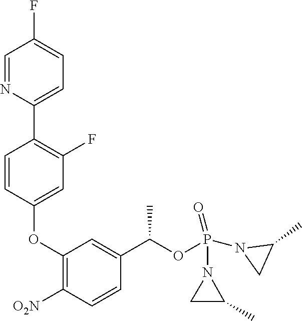 [SMILES: C[C@H](OP(=O)(N1C[C@H]1C)N1C[C@H]1C)c1ccc([N+](=O)[O-])c(Oc2ccc(-c3ccc(F)cn3)c(F)c2)c1]

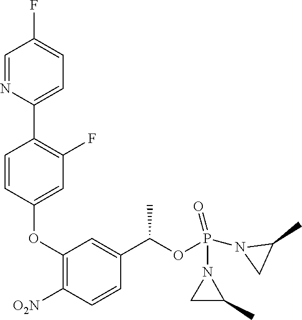 [SMILES: C[C@H](OP(=O)(N1C[C@@H]1C)N1C[C@@H]1C)c1ccc([N+](=O)[O-])c(Oc2ccc(-c3ccc(F)cn3)c(F)c2)c1]

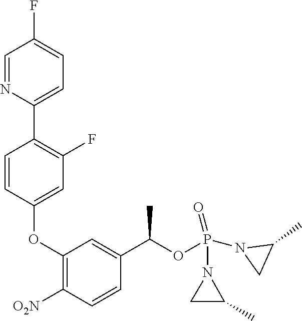 [SMILES: C[C@@H]1CN1P(=O)(O[C@H](C)c1ccc([N+](=O)[O-])c(Oc2ccc(-c3ccc(F)cn3)c(F)c2)c1)N1C[C@H]1C]

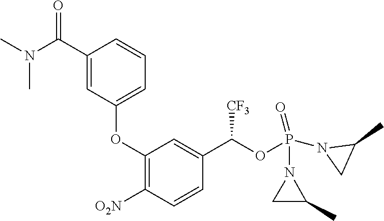

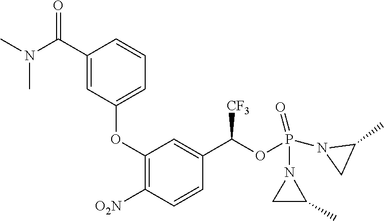

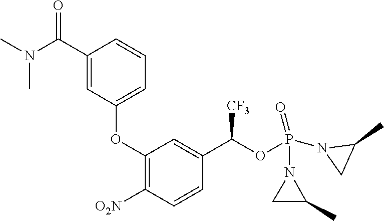

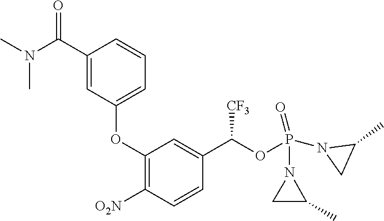

TABLE A10
List of compounds

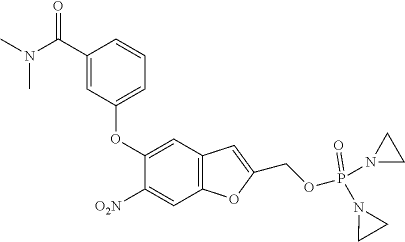 [SMILES: CN(C)C(=O)c1cccc(Oc2cc3cc(COP(=O)(N4CC4)N4CC4)oc3cc2[N+](=O)[O-])c1]

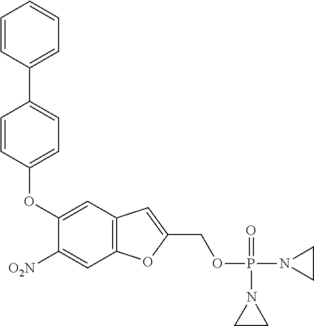 [SMILES: O=[N+]([O-])c1cc2oc(COP(=O)(N3CC3)N3CC3)cc2cc1Oc1ccc(-c2ccccc2)cc1]

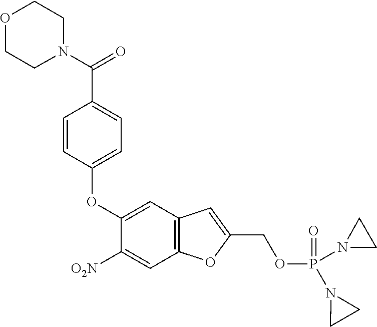 [SMILES: O=C(c1ccc(Oc2cc3cc(COP(=O)(N4CC4)N4CC4)oc3cc2[N+](=O)[O-])cc1)N1CCOCC1]

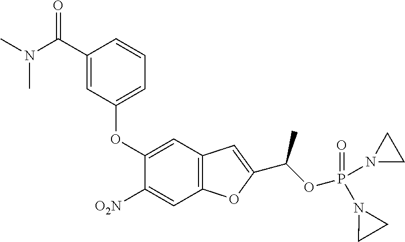 [SMILES: C[C@@H](OP(=O)(N1CC1)N1CC1)c1cc2cc(Oc3cccc(C(=O)N(C)C)c3)c([N+](=O)[O-])cc2o1]

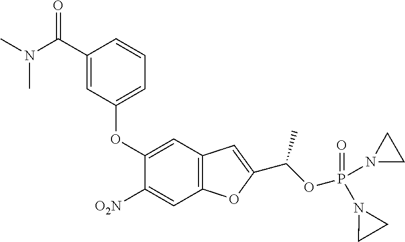 [SMILES: C[C@H](OP(=O)(N1CC1)N1CC1)c1cc2cc(Oc3cccc(C(=O)N(C)C)c3)c([N+](=O)[O-])cc2o1]

TABLE A11
List of compounds

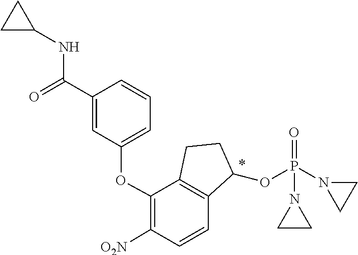 [SMILES: O=C(NC1CC1)c1cccc(Oc2c([N+](=O)[O-])ccc3c2CCC3OP(=O)(N2CC2)N2CC2)c1]

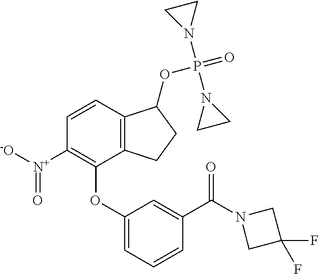

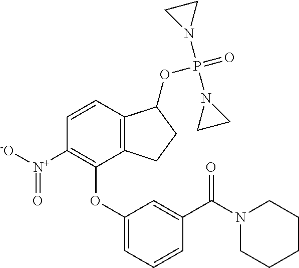

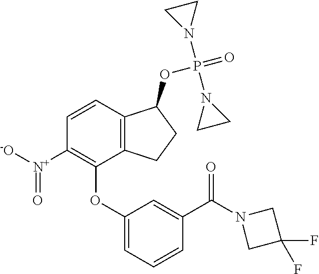

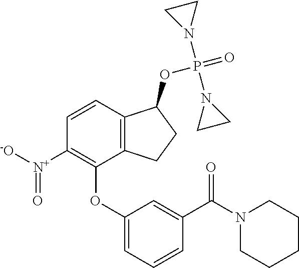

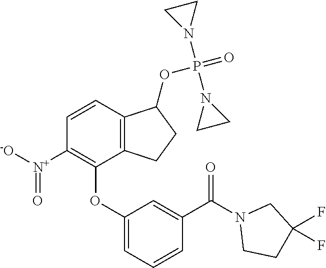

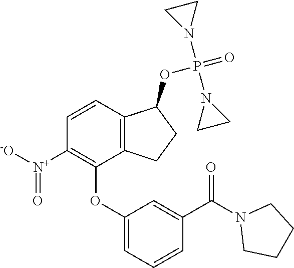

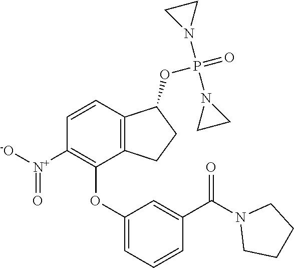

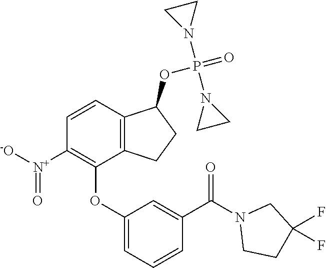

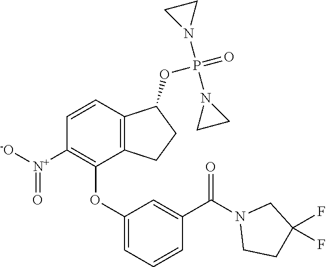

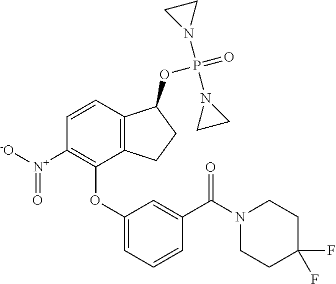

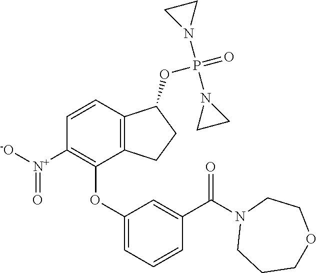

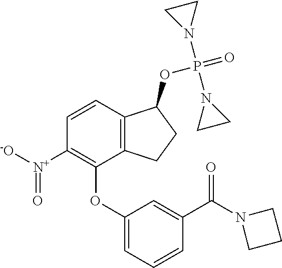

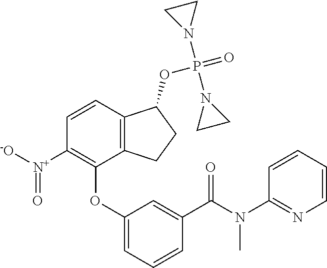

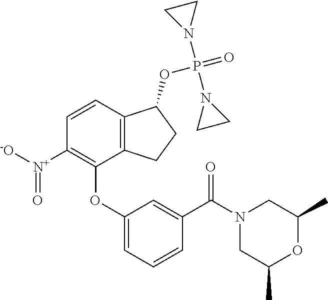

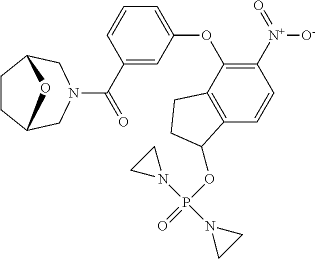 [SMILES: O=C(c1cccc(Oc2c([N+](=O)[O-])ccc3c2CCC3OP(=O)(N2CC2)N2CC2)c1)N1C[C@H]2CC[C@@H](C1)O2]

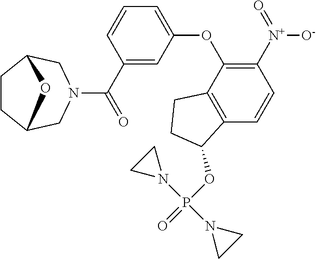 [SMILES: O=C(c1cccc(Oc2c([N+](=O)[O-])ccc3c2CC[C@H]3OP(=O)(N2CC2)N2CC2)c1)N1C[C@H]2CC[C@@H](C1)O2]

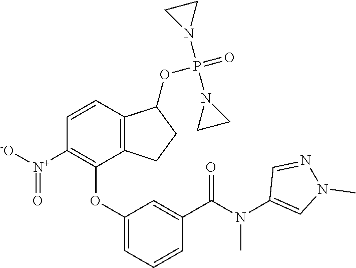

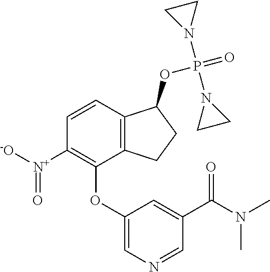

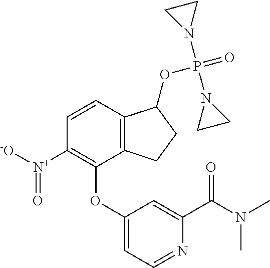

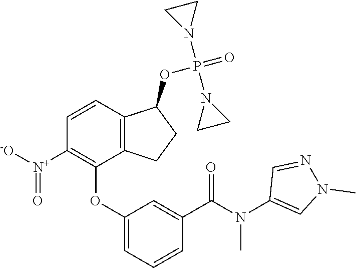

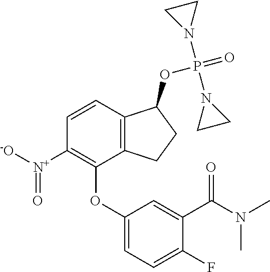

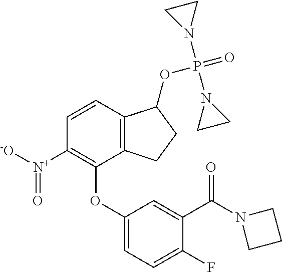

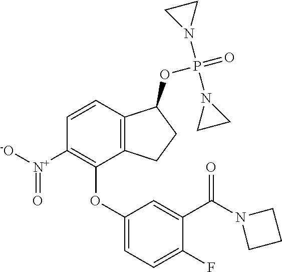

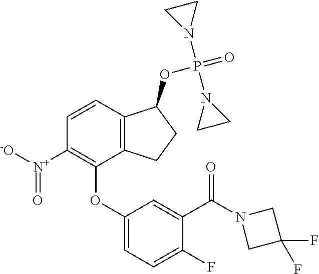

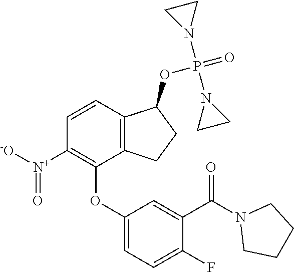

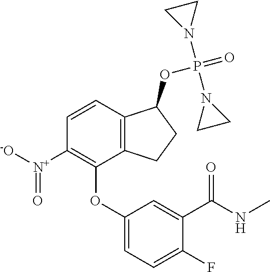

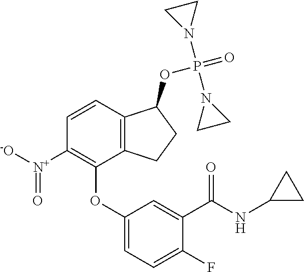

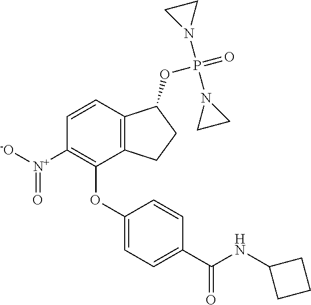

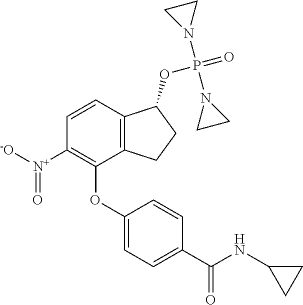

TABLE A12
List of compounds

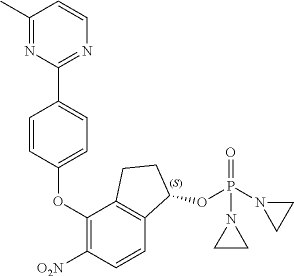

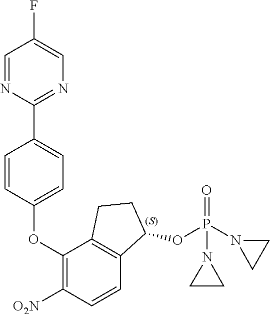

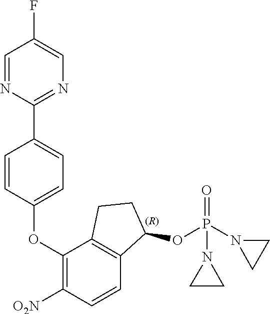

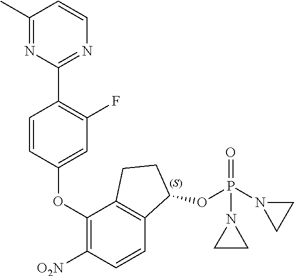

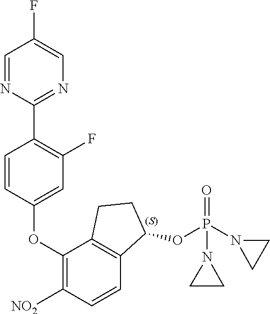

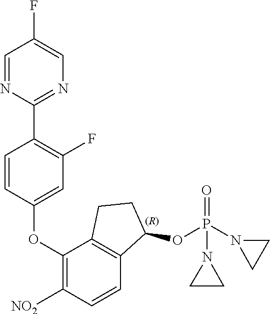

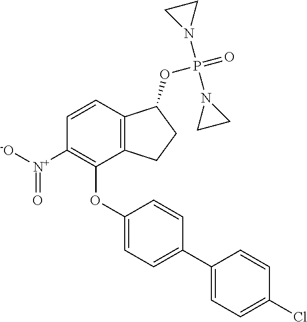

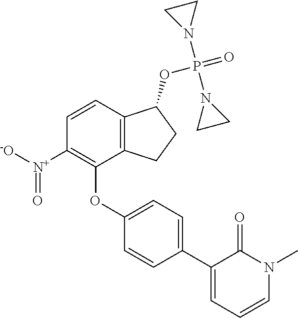

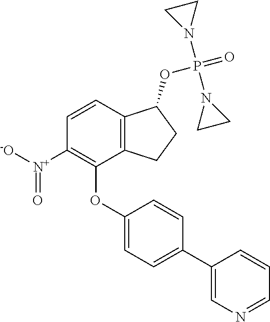

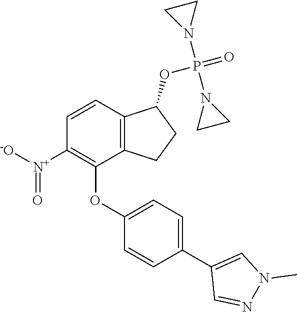

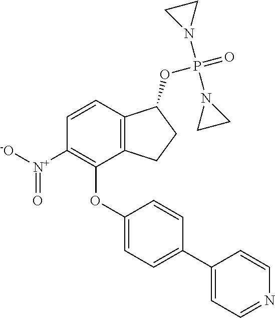

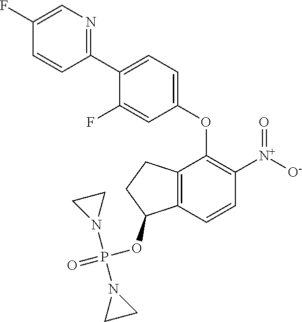 [SMILES: O=[N+]([O-])c1ccc2c(c1Oc1ccc(-c3ccc(F)cn3)c(F)c1)CC[C@@H]2OP(=O)(N1CC1)N1CC1]

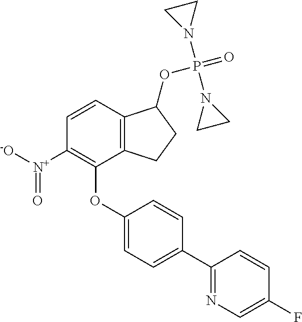

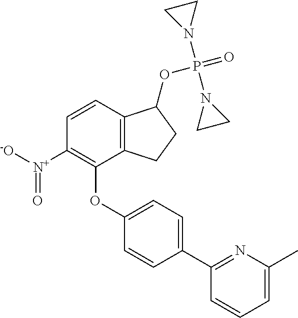

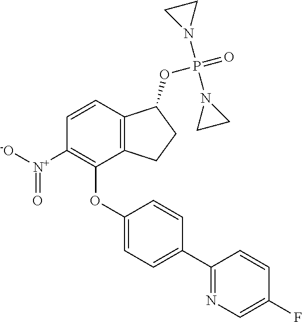

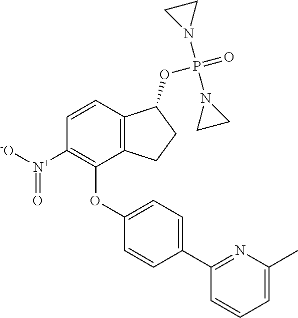

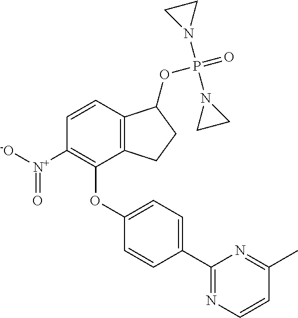

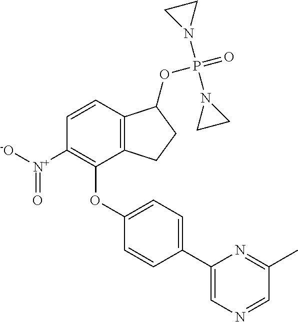

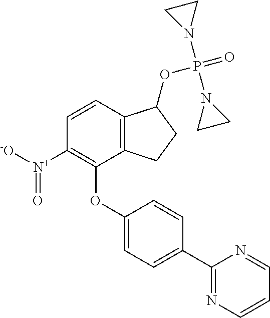

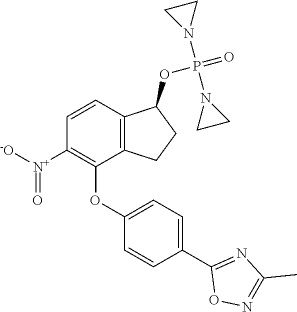

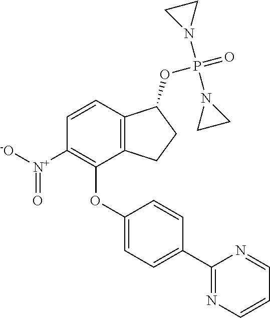

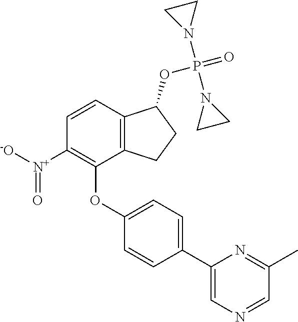

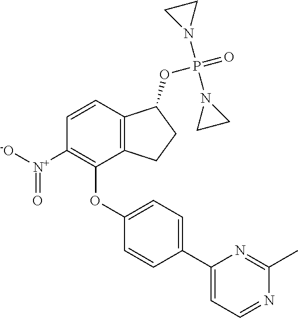

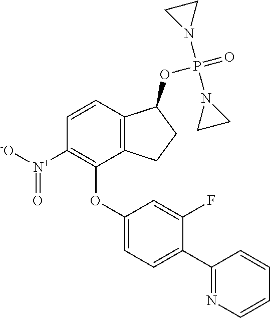

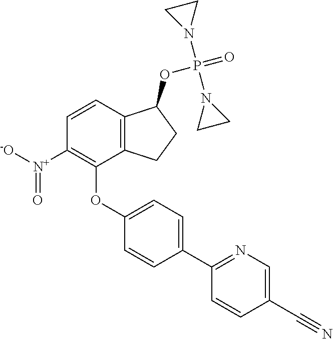

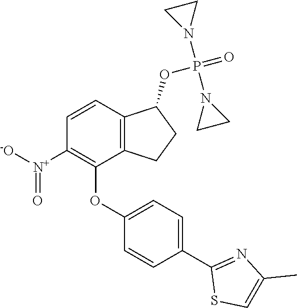

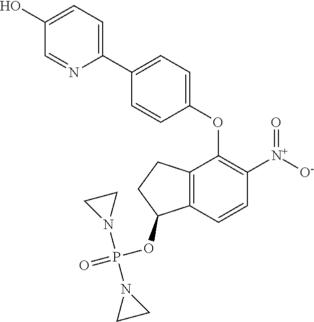 [SMILES: O=[N+]([O-])c1ccc2c(c1Oc1ccc(-c3ccc(O)cn3)cc1)CC[C@@H]2OP(=O)(N1CC1)N1CC1]

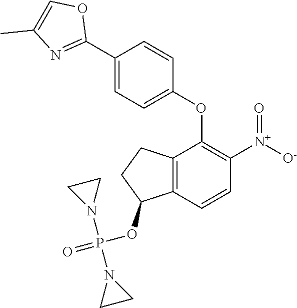 [SMILES: Cc1coc(-c2ccc(Oc3c([N+](=O)[O-])ccc4c3CC[C@@H]4OP(=O)(N3CC3)N3CC3)cc2)n1]

TABLE A13
List of compounds

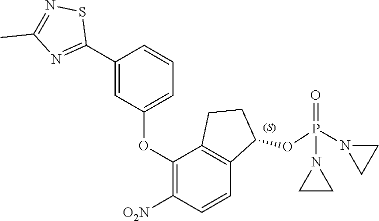

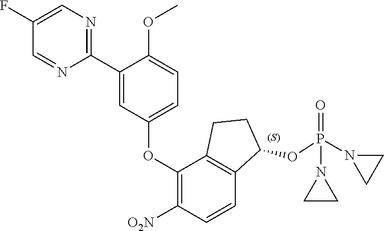

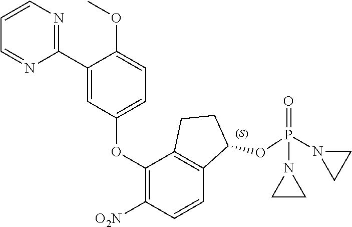

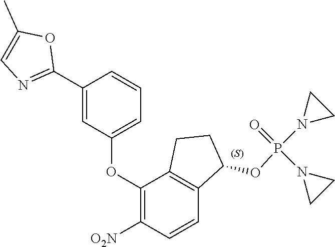

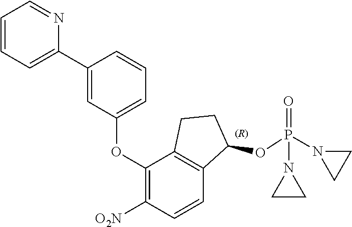

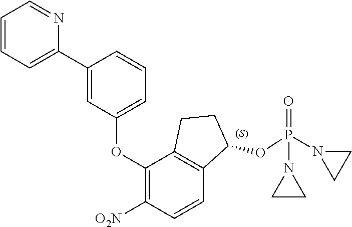

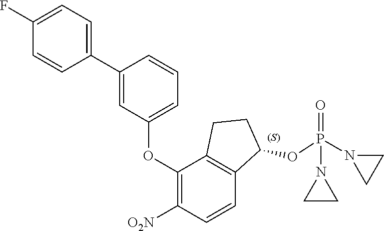

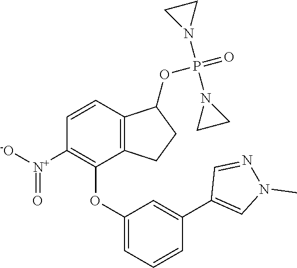

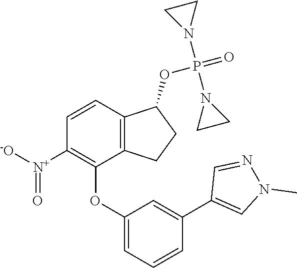

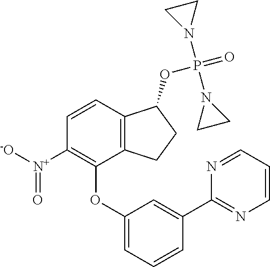

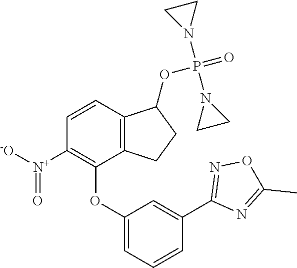

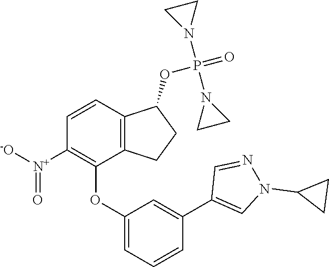

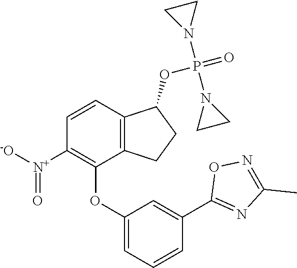

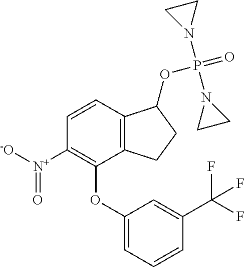

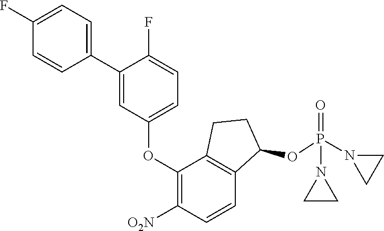 [SMILES: O=[N+]([O-])c1ccc2c(c1Oc1ccc(F)c(-c3ccc(F)cc3)c1)CC[C@H]2OP(=O)(N1CC1)N1CC1]

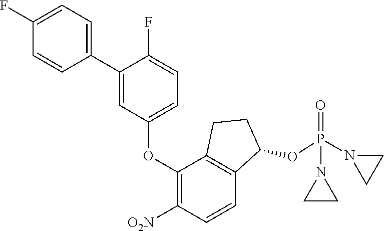 [SMILES: O=[N+]([O-])c1ccc2c(c1Oc1ccc(F)c(-c3ccc(F)cc3)c1)CC[C@@H]2OP(=O)(N1CC1)N1CC1]

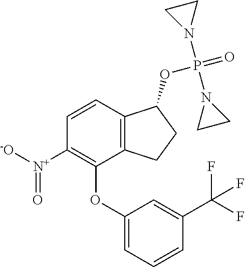

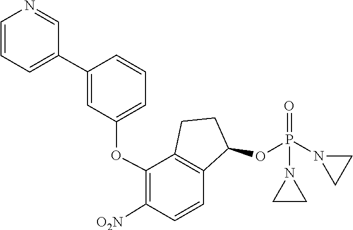 [SMILES: O=[N+]([O-])c1ccc2c(c1Oc1cccc(-c3cccnc3)c1)CC[C@H]2OP(=O)(N1CC1)N1CC1]

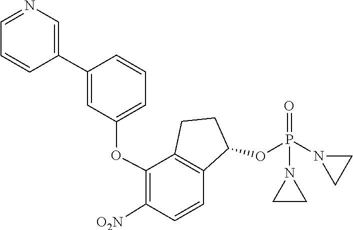 [SMILES: O=[N+]([O-])c1ccc2c(c1Oc1cccc(-c3cccnc3)c1)CC[C@@H]2OP(=O)(N1CC1)N1CC1]

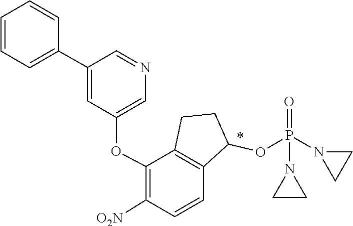 [SMILES: O=[N+]([O-])c1ccc2c(c1Oc1cncc(-c3ccccc3)c1)CCC2OP(=O)(N1CC1)N1CC1]

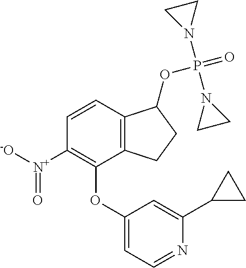

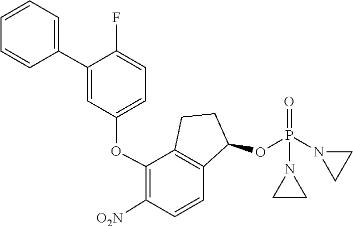 [SMILES: O=[N+]([O-])c1ccc2c(c1Oc1ccc(F)c(-c3ccccc3)c1)CC[C@H]2OP(=O)(N1CC1)N1CC1]

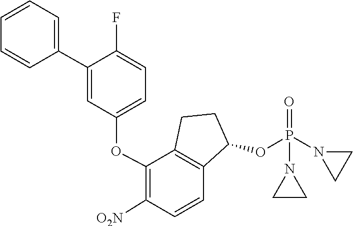 [SMILES: O=[N+]([O-])c1ccc2c(c1Oc1ccc(F)c(-c3ccccc3)c1)CC[C@@H]2OP(=O)(N1CC1)N1CC1]

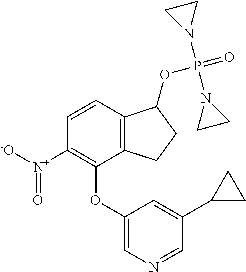

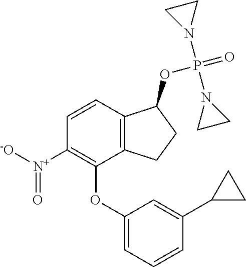

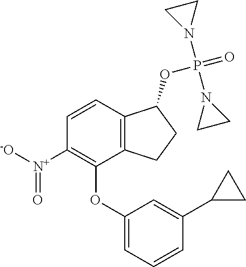

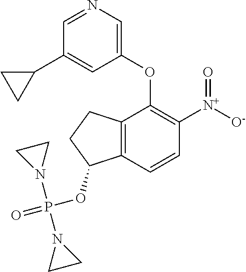 [SMILES: O=[N+]([O-])c1ccc2c(c1Oc1cncc(C3CC3)c1)CC[C@H]2OP(=O)(N1CC1)N1CC1]

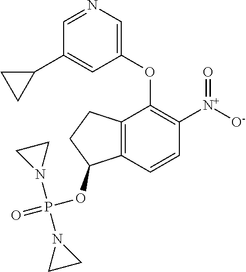 [SMILES: O=[N+]([O-])c1ccc2c(c1Oc1cncc(C3CC3)c1)CC[C@@H]2OP(=O)(N1CC1)N1CC1]

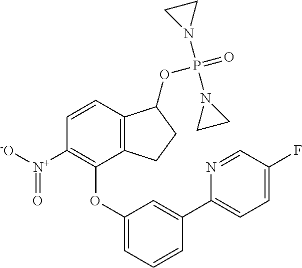

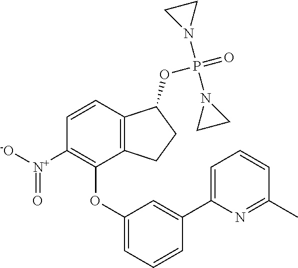

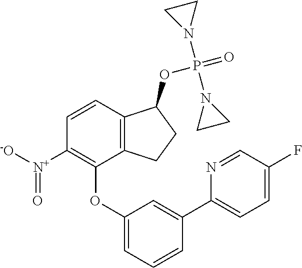

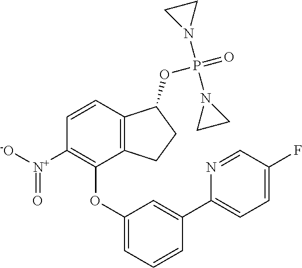

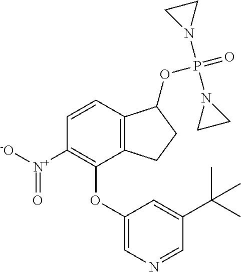

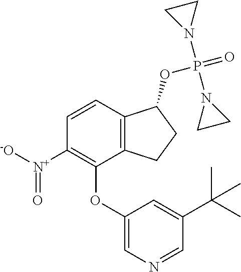

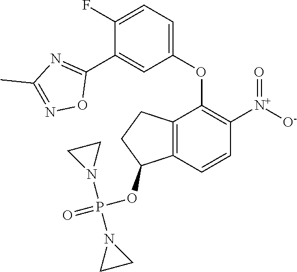 [SMILES: Cc1noc(-c2cc(Oc3c([N+](=O)[O-])ccc4c3CC[C@@H]4OP(=O)(N3CC3)N3CC3)ccc2F)n1]

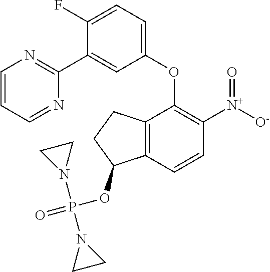 [SMILES: O=[N+]([O-])c1ccc2c(c1Oc1ccc(F)c(-c3ncccn3)c1)CC[C@@H]2OP(=O)(N1CC1)N1CC1]

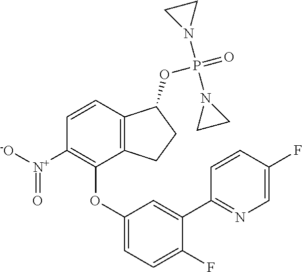

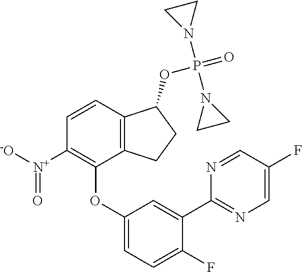

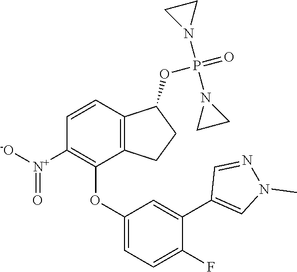

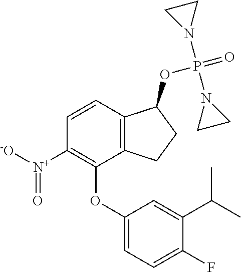

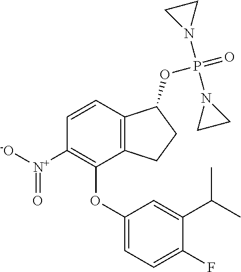

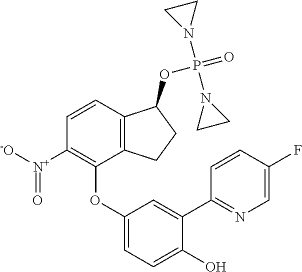

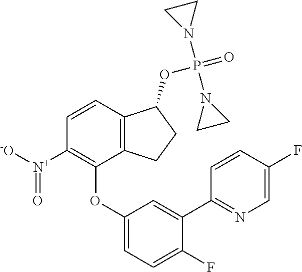

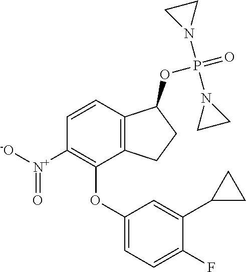

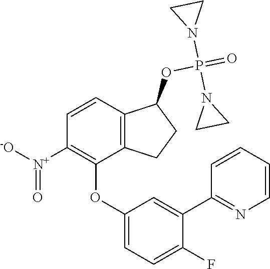

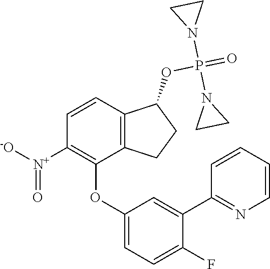

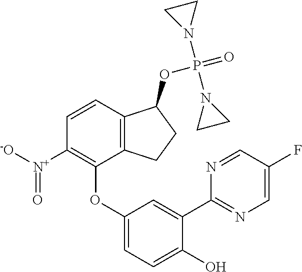

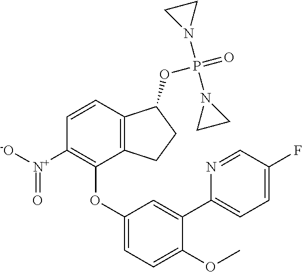

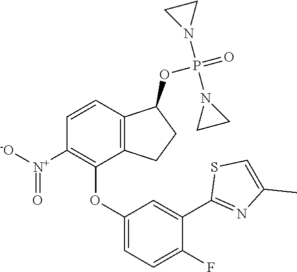

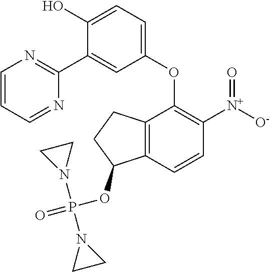 [SMILES: O=[N+]([O-])c1ccc2c(c1Oc1ccc(O)c(-c3ncccn3)c1)CC[C@@H]2OP(=O)(N1CC1)N1CC1]

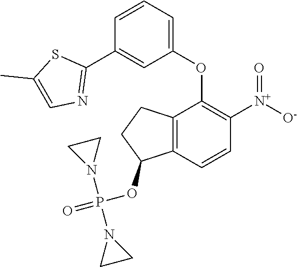 [SMILES: Cc1cnc(-c2cccc(Oc3c([N+](=O)[O-])ccc4c3CC[C@@H]4OP(=O)(N3CC3)N3CC3)c2)s1]

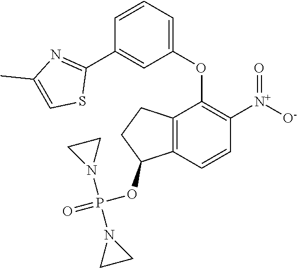 [SMILES: Cc1csc(-c2cccc(Oc3c([N+](=O)[O-])ccc4c3CC[C@@H]4OP(=O)(N3CC3)N3CC3)c2)n1]

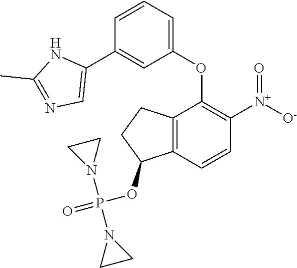 [SMILES: Cc1ncc(-c2cccc(Oc3c([N+](=O)[O-])ccc4c3CC[C@@H]4OP(=O)(N3CC3)N3CC3)c2)[nH]1]

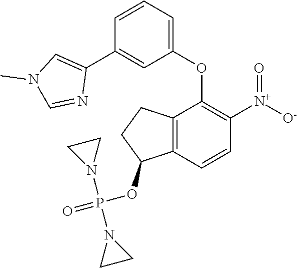 [SMILES: Cn1cnc(-c2cccc(Oc3c([N+](=O)[O-])ccc4c3CC[C@@H]4OP(=O)(N3CC3)N3CC3)c2)c1]

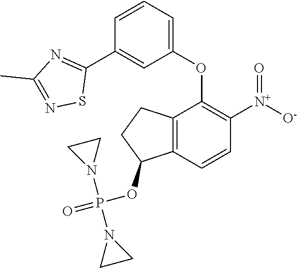 [SMILES: Cc1nsc(-c2cccc(Oc3c([N+](=O)[O-])ccc4c3CC[C@@H]4OP(=O)(N3CC3)N3CC3)c2)n1]

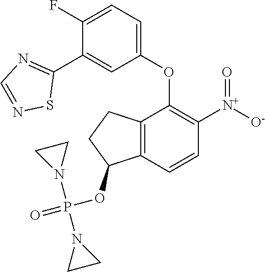 [SMILES: O=[N+]([O-])c1ccc2c(c1Oc1ccc(F)c(-c3ncns3)c1)CC[C@@H]2OP(=O)(N1CC1)N1CC1]

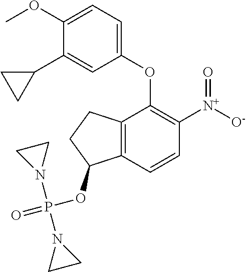 [SMILES: COc1ccc(Oc2c([N+](=O)[O-])ccc3c2CC[C@@H]3OP(=O)(N2CC2)N2CC2)cc1C1CC1]

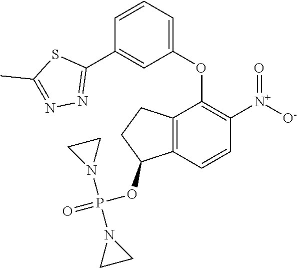 [SMILES: Cc1nnc(-c2cccc(Oc3c([N+](=O)[O-])ccc4c3CC[C@@H]4OP(=O)(N3CC3)N3CC3)c2)s1]

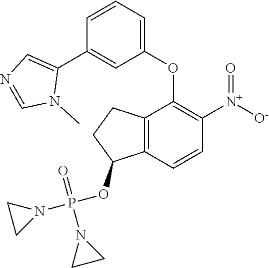 [SMILES: Cn1cncc1-c1cccc(Oc2c([N+](=O)[O-])ccc3c2CC[C@@H]3OP(=O)(N2CC2)N2CC2)c1]

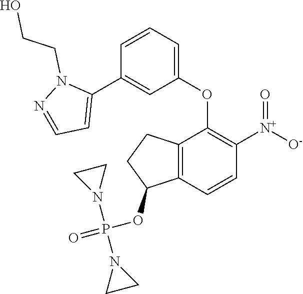 [SMILES: O=[N+]([O-])c1ccc2c(c1Oc1cccc(-c3ccnn3CCO)c1)CC[C@@H]2OP(=O)(N1CC1)N1CC1]

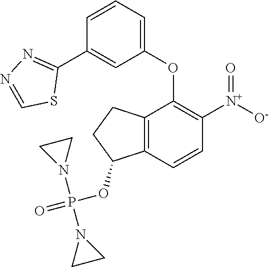 [SMILES: O=[N+]([O-])c1ccc2c(c1Oc1cccc(-c3nncs3)c1)CC[C@H]2OP(=O)(N1CC1)N1CC1]

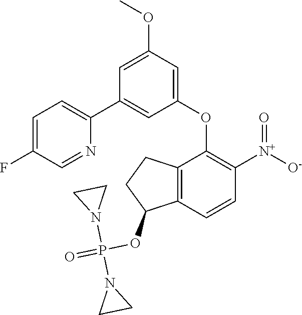 [SMILES: COc1cc(Oc2c([N+](=O)[O-])ccc3c2CC[C@@H]3OP(=O)(N2CC2)N2CC2)cc(-c2ccc(F)cn2)c1]

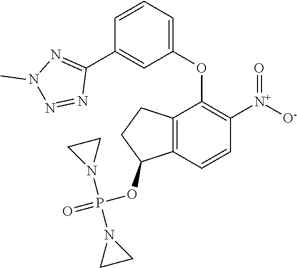 [SMILES: Cn1nnc(-c2cccc(Oc3c([N+](=O)[O-])ccc4c3CC[C@@H]4OP(=O)(N3CC3)N3CC3)c2)n1]

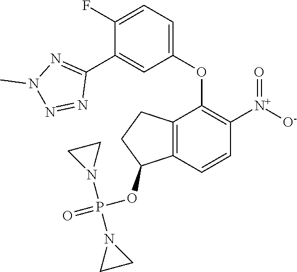 [SMILES: Cn1nnc(-c2cc(Oc3c([N+](=O)[O-])ccc4c3CC[C@@H]4OP(=O)(N3CC3)N3CC3)ccc2F)n1]

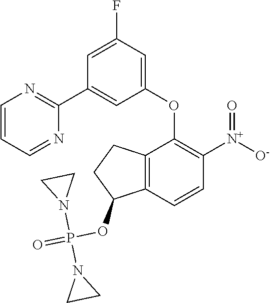 [SMILES: O=[N+]([O-])c1ccc2c(c1Oc1cc(F)cc(-c3ncccn3)c1)CC[C@@H]2OP(=O)(N1CC1)N1CC1]

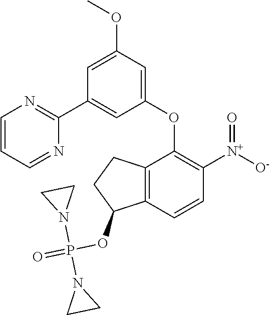 [SMILES: COc1cc(Oc2c([N+](=O)[O-])ccc3c2CC[C@@H]3OP(=O)(N2CC2)N2CC2)cc(-c2ncccn2)c1]

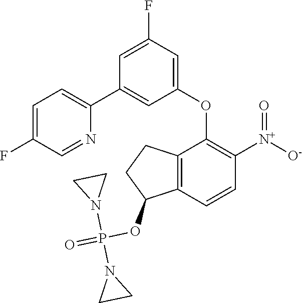 [SMILES: O=[N+]([O-])c1ccc2c(c1Oc1cc(F)cc(-c3ccc(F)cn3)c1)CC[C@@H]2OP(=O)(N1CC1)N1CC1]

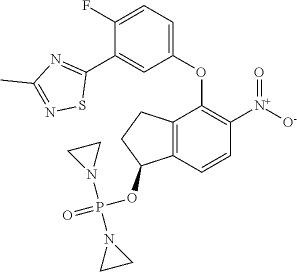 [SMILES: Cc1nsc(-c2cc(Oc3c([N+](=O)[O-])ccc4c3CC[C@@H]4OP(=O)(N3CC3)N3CC3)ccc2F)n1]

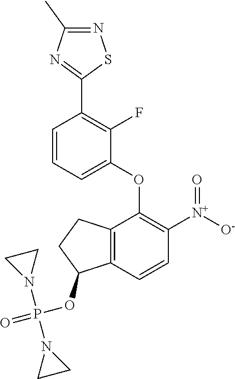 [SMILES: Cc1nsc(-c2cccc(Oc3c([N+](=O)[O-])ccc4c3CC[C@@H]4OP(=O)(N3CC3)N3CC3)c2F)n1]

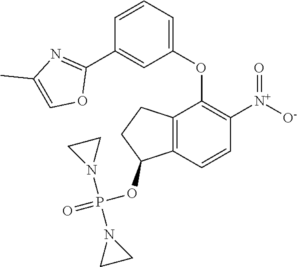 [SMILES: Cc1coc(-c2cccc(Oc3c([N+](=O)[O-])ccc4c3CC[C@@H]4OP(=O)(N3CC3)N3CC3)c2)n1]

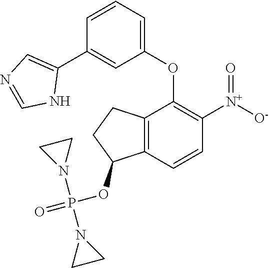 [SMILES: O=[N+]([O-])c1ccc2c(c1Oc1cccc(-c3cnc[nH]3)c1)CC[C@@H]2OP(=O)(N1CC1)N1CC1]

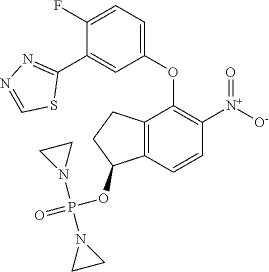 [SMILES: O=[N+]([O-])c1ccc2c(c1Oc1ccc(F)c(-c3nncs3)c1)CC[C@@H]2OP(=O)(N1CC1)N1CC1]

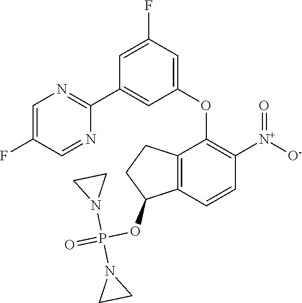 [SMILES: O=[N+]([O-])c1ccc2c(c1Oc1cc(F)cc(-c3ncc(F)cn3)c1)CC[C@@H]2OP(=O)(N1CC1)N1CC1]

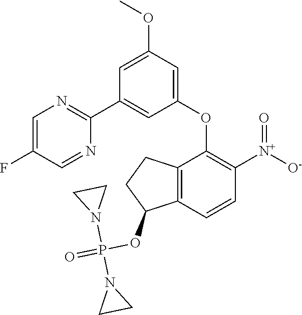 [SMILES: COc1cc(Oc2c([N+](=O)[O-])ccc3c2CC[C@@H]3OP(=O)(N2CC2)N2CC2)cc(-c2ncc(F)cn2)c1]

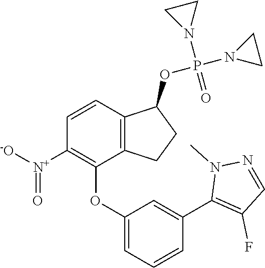

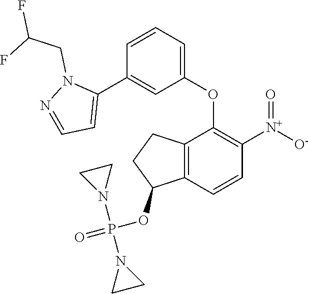 [SMILES: O=[N+]([O-])c1ccc2c(c1Oc1cccc(-c3ccnn3CC(F)F)c1)CC[C@@H]2OP(=O)(N1CC1)N1CC1]

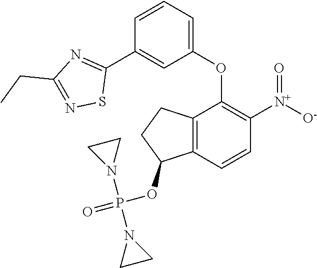 [SMILES: CCc1nsc(-c2cccc(Oc3c([N+](=O)[O-])ccc4c3CC[C@@H]4OP(=O)(N3CC3)N3CC3)c2)n1]

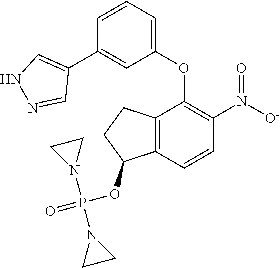 [SMILES: O=[N+]([O-])c1ccc2c(c1Oc1cccc(-c3cn[nH]c3)c1)CC[C@@H]2OP(=O)(N1CC1)N1CC1]

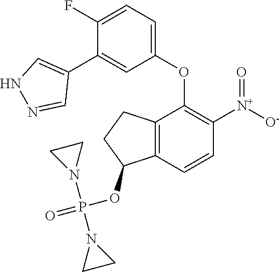 [SMILES: O=[N+]([O-])c1ccc2c(c1Oc1ccc(F)c(-c3cn[nH]c3)c1)CC[C@@H]2OP(=O)(N1CC1)N1CC1]

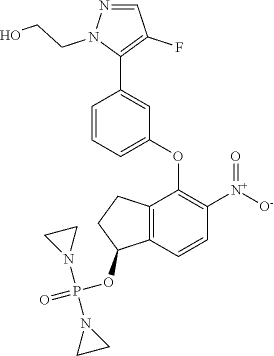 [SMILES: O=[N+]([O-])c1ccc2c(c1Oc1cccc(-c3c(F)cnn3CCO)c1)CC[C@@H]2OP(=O)(N1CC1)N1CC1]

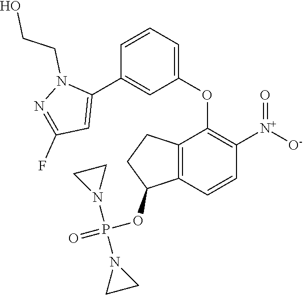 [SMILES: O=[N+]([O-])c1ccc2c(c1Oc1cccc(-c3cc(F)nn3CCO)c1)CC[C@@H]2OP(=O)(N1CC1)N1CC1]

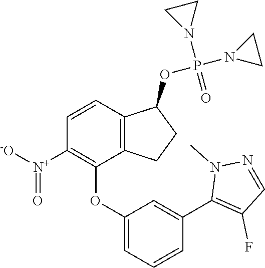

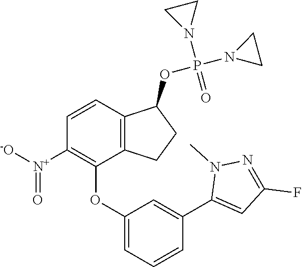

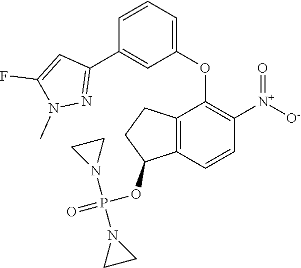 [SMILES: Cn1nc(-c2cccc(Oc3c([N+](=O)[O-])ccc4c3CC[C@@H]4OP(=O)(N3CC3)N3CC3)c2)cc1F]

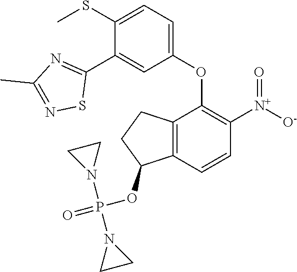 [SMILES: CSc1ccc(Oc2c([N+](=O)[O-])ccc3c2CC[C@@H]3OP(=O)(N2CC2)N2CC2)cc1-c1nc(C)ns1]

TABLE A14
List of compounds

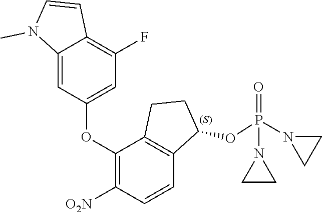

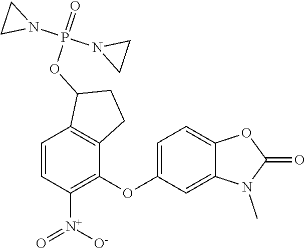 [SMILES: Cn1c(=O)oc2ccc(Oc3c([N+](=O)[O-])ccc4c3CCC4OP(=O)(N3CC3)N3CC3)cc21]

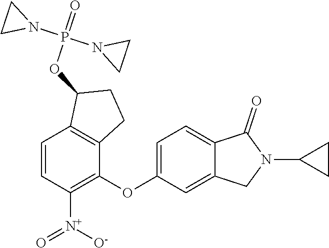 [SMILES: O=C1c2ccc(Oc3c([N+](=O)[O-])ccc4c3CC[C@@H]4OP(=O)(N3CC3)N3CC3)cc2CN1C1CC1]

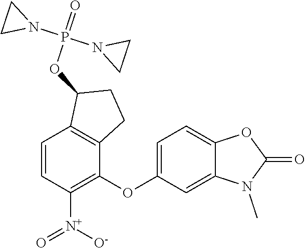 [SMILES: Cn1c(=O)oc2ccc(Oc3c([N+](=O)[O-])ccc4c3CC[C@@H]4OP(=O)(N3CC3)N3CC3)cc21]

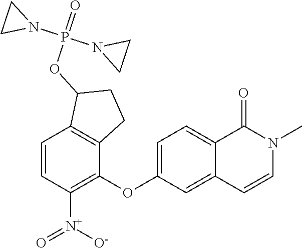 [SMILES: Cn1ccc2cc(Oc3c([N+](=O)[O-])ccc4c3CCC4OP(=O)(N3CC3)N3CC3)ccc2c1=O]

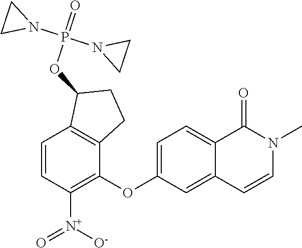 [SMILES: Cn1ccc2cc(Oc3c([N+](=O)[O-])ccc4c3CC[C@@H]4OP(=O)(N3CC3)N3CC3)ccc2c1=O]

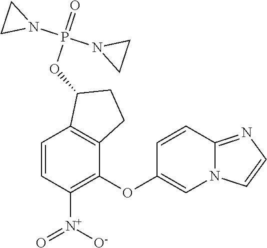 [SMILES: O=[N+]([O-])c1ccc2c(c1Oc1ccc3nccn3c1)CC[C@H]2OP(=O)(N1CC1)N1CC1]

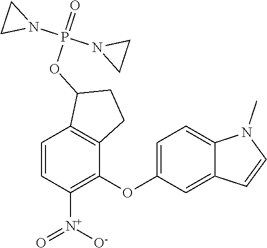 [SMILES: Cn1ccc2cc(Oc3c([N+](=O)[O-])ccc4c3CCC4OP(=O)(N3CC3)N3CC3)ccc21]

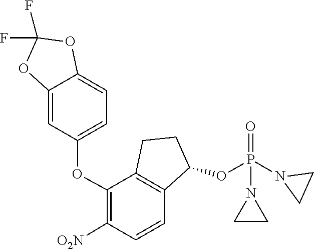 [SMILES: O=[N+]([O-])c1ccc2c(c1Oc1ccc3c(c1)OC(F)(F)O3)CC[C@@H]2OP(=O)(N1CC1)N1CC1]

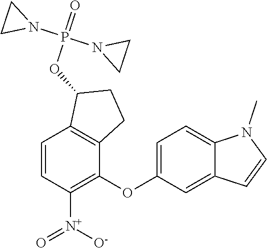 [SMILES: Cn1ccc2cc(Oc3c([N+](=O)[O-])ccc4c3CC[C@H]4OP(=O)(N3CC3)N3CC3)ccc21]

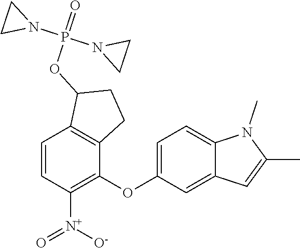 [SMILES: Cc1cc2cc(Oc3c([N+](=O)[O-])ccc4c3CCC4OP(=O)(N3CC3)N3CC3)ccc2n1C]

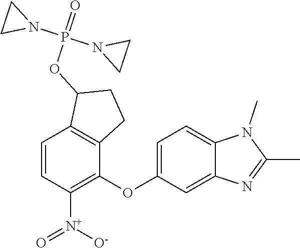 [SMILES: Cc1nc2cc(Oc3c([N+](=O)[O-])ccc4c3CCC4OP(=O)(N3CC3)N3CC3)ccc2n1C]

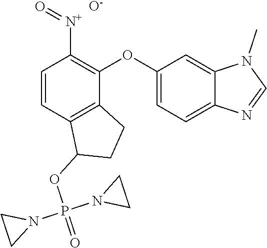

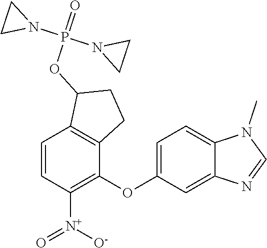 [SMILES: Cn1cnc2cc(Oc3c([N+](=O)[O-])ccc4c3CCC4OP(=O)(N3CC3)N3CC3)ccc21]

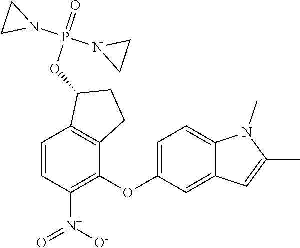 [SMILES: Cc1cc2cc(Oc3c([N+](=O)[O-])ccc4c3CC[C@H]4OP(=O)(N3CC3)N3CC3)ccc2n1C]

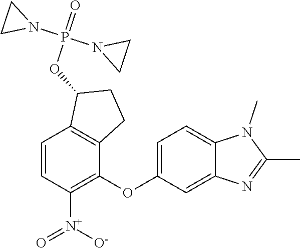 [SMILES: Cc1nc2cc(Oc3c([N+](=O)[O-])ccc4c3CC[C@H]4OP(=O)(N3CC3)N3CC3)ccc2n1C]

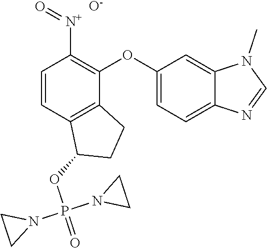

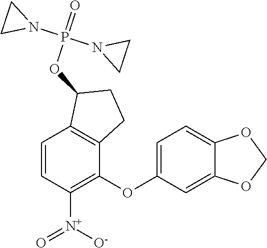 [SMILES: O=[N+]([O-])c1ccc2c(c1Oc1ccc3c(c1)OCO3)CC[C@@H]2OP(=O)(N1CC1)N1CC1]

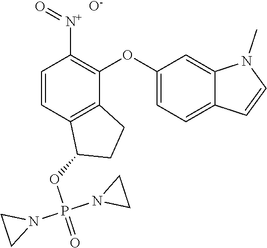

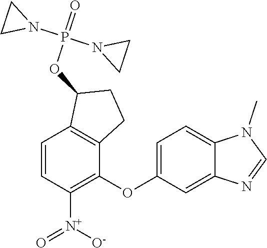 [SMILES: Cn1cnc2cc(Oc3c([N+](=O)[O-])ccc4c3CC[C@@H]4OP(=O)(N3CC3)N3CC3)ccc21]

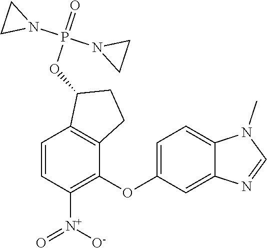 [SMILES: Cn1cnc2cc(Oc3c([N+](=O)[O-])ccc4c3CC[C@H]4OP(=O)(N3CC3)N3CC3)ccc21]

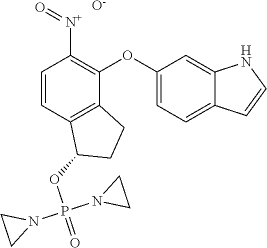

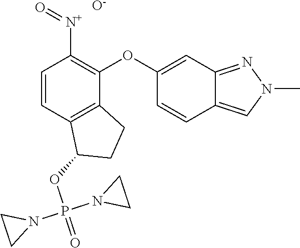

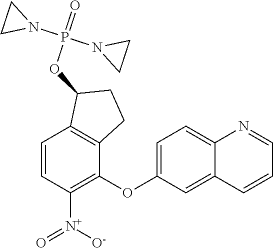 [SMILES: O=[N+]([O-])c1ccc2c(c1Oc1ccc3ncccc3c1)CC[C@@H]2OP(=O)(N1CC1)N1CC1]

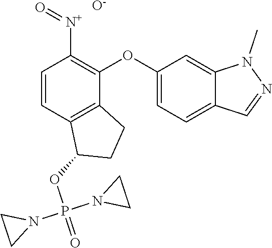

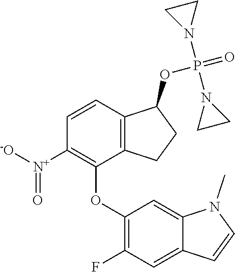

TABLE A15
List of compounds

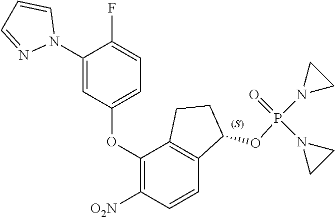

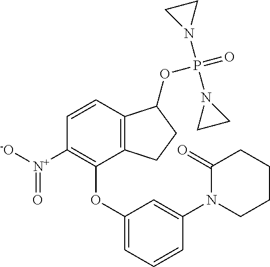

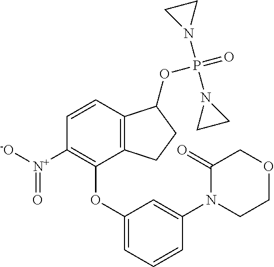

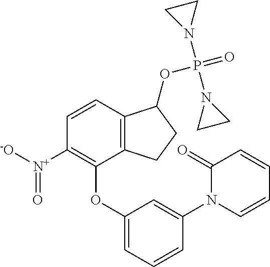

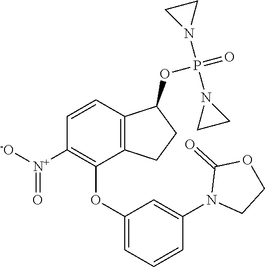

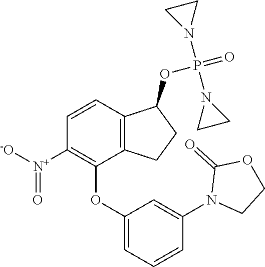

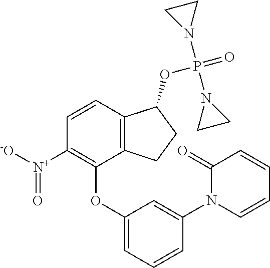

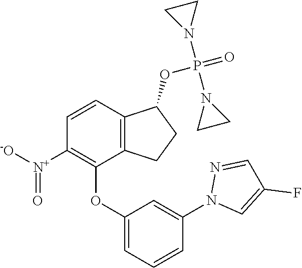

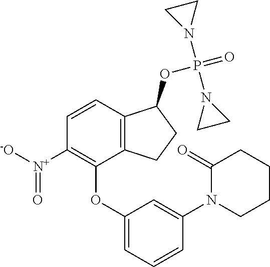

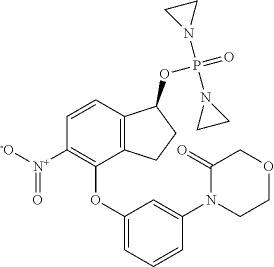

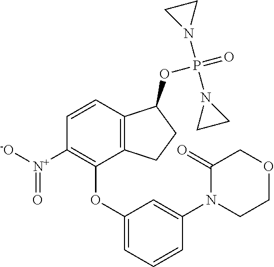

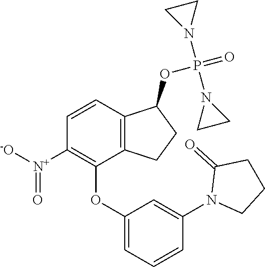

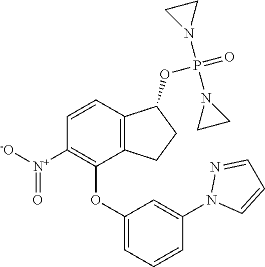

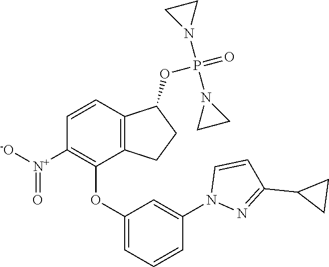

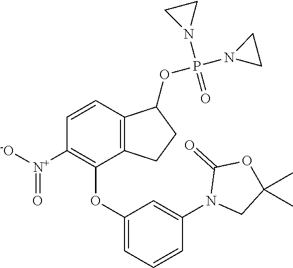

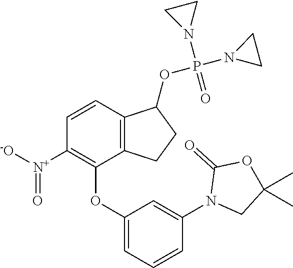

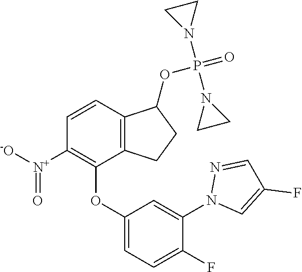

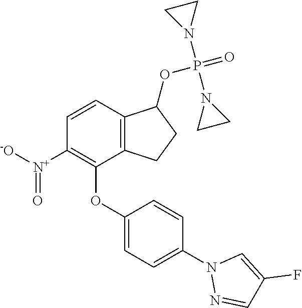

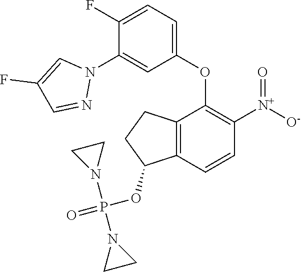 [SMILES: O=[N+]([O-])c1ccc2c(c1Oc1ccc(F)c(-n3cc(F)cn3)c1)CC[C@H]2OP(=O)(N1CC1)N1CC1]

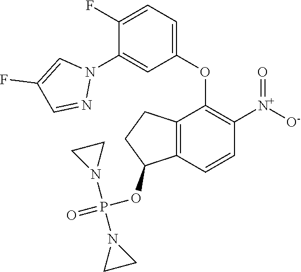 [SMILES: O=[N+]([O-])c1ccc2c(c1Oc1ccc(F)c(-n3cc(F)cn3)c1)CC[C@@H]2OP(=O)(N1CC1)N1CC1]

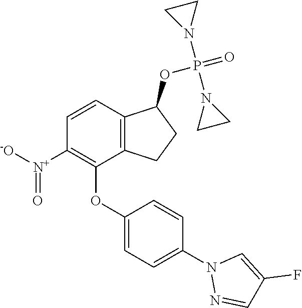

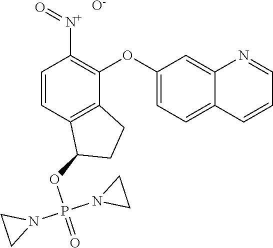

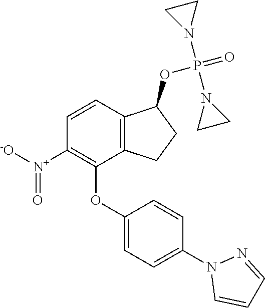

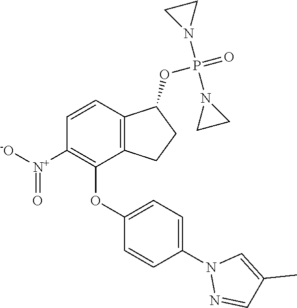

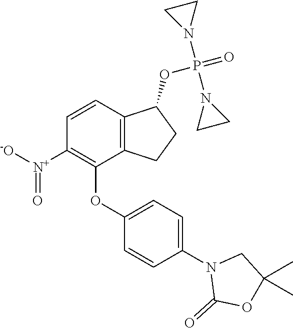

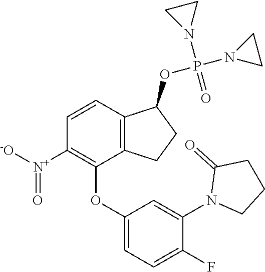

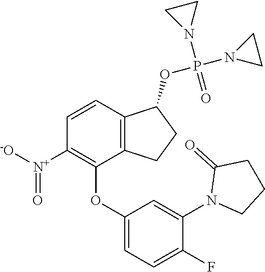

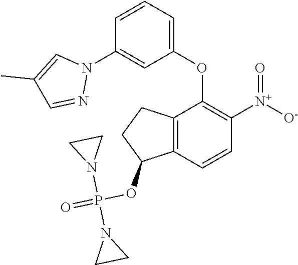 [SMILES: Cc1cnn(-c2cccc(Oc3c([N+](=O)[O-])ccc4c3CC[C@@H]4OP(=O)(N3CC3)N3CC3)c2)c1]

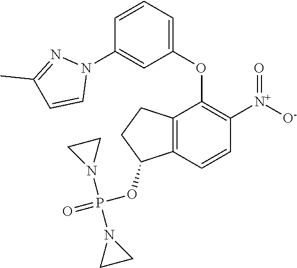 [SMILES: Cc1ccn(-c2cccc(Oc3c([N+](=O)[O-])ccc4c3CC[C@H]4OP(=O)(N3CC3)N3CC3)c2)n1]

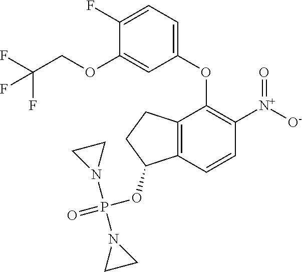 [SMILES: O=[N+]([O-])c1ccc2c(c1Oc1ccc(F)c(OCC(F)(F)F)c1)CC[C@H]2OP(=O)(N1CC1)N1CC1]

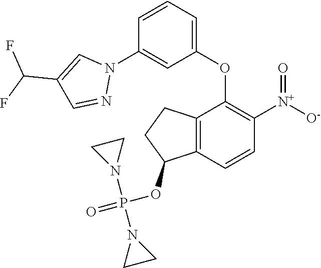 [SMILES: O=[N+]([O-])c1ccc2c(c1Oc1cccc(-n3cc(C(F)F)cn3)c1)CC[C@@H]2OP(=O)(N1CC1)N1CC1]

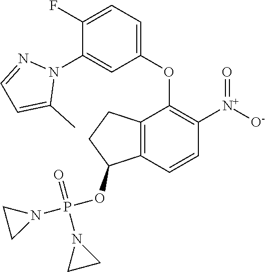 [SMILES: Cc1ccnn1-c1cc(Oc2c([N+](=O)[O-])ccc3c2CC[C@@H]3OP(=O)(N2CC2)N2CC2)ccc1F]

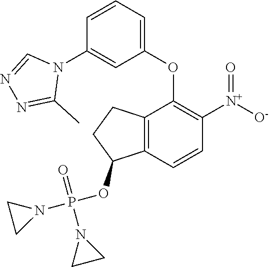 [SMILES: Cc1nncn1-c1cccc(Oc2c([N+](=O)[O-])ccc3c2CC[C@@H]3OP(=O)(N2CC2)N2CC2)c1]

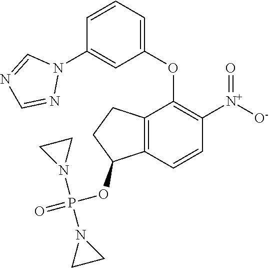 [SMILES: O=[N+]([O-])c1ccc2c(c1Oc1cccc(-n3cncn3)c1)CC[C@@H]2OP(=O)(N1CC1)N1CC1]

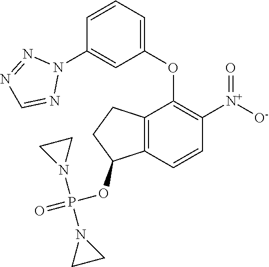 [SMILES: O=[N+]([O-])c1ccc2c(c1Oc1cccc(-n3ncnn3)c1)CC[C@@H]2OP(=O)(N1CC1)N1CC1]

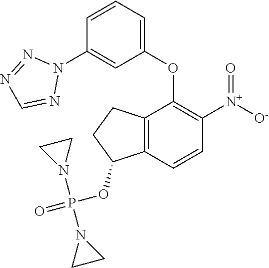 [SMILES: O=[N+]([O-])c1ccc2c(c1Oc1cccc(-n3ncnn3)c1)CC[C@H]2OP(=O)(N1CC1)N1CC1]

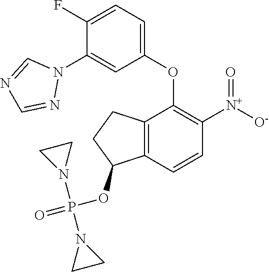 [SMILES: O=[N+]([O-])c1ccc2c(c1Oc1ccc(F)c(-n3cncn3)c1)CC[C@@H]2OP(=O)(N1CC1)N1CC1]

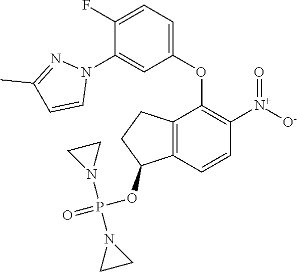 [SMILES: Cc1ccn(-c2cc(Oc3c([N+](=O)[O-])ccc4c3CC[C@@H]4OP(=O)(N3CC3)N3CC3)ccc2F)n1]

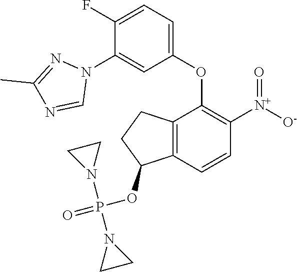 [SMILES: Cc1ncn(-c2cc(Oc3c([N+](=O)[O-])ccc4c3CC[C@@H]4OP(=O)(N3CC3)N3CC3)ccc2F)n1]

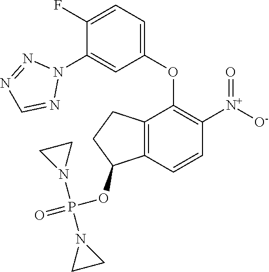 [SMILES: O=[N+]([O-])c1ccc2c(c1Oc1ccc(F)c(-n3ncnn3)c1)CC[C@@H]2OP(=O)(N1CC1)N1CC1]

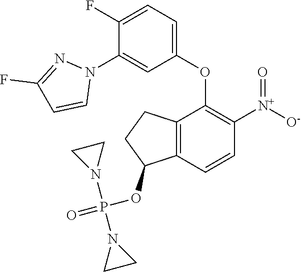 [SMILES: O=[N+]([O-])c1ccc2c(c1Oc1ccc(F)c(-n3ccc(F)n3)c1)CC[C@@H]2OP(=O)(N1CC1)N1CC1]

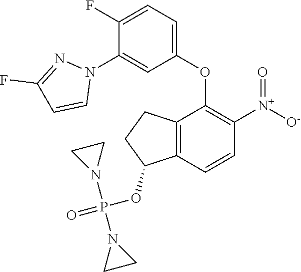 [SMILES: O=[N+]([O-])c1ccc2c(c1Oc1ccc(F)c(-n3ccc(F)n3)c1)CC[C@H]2OP(=O)(N1CC1)N1CC1]

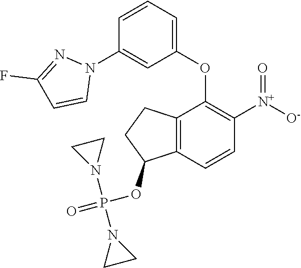 [SMILES: O=[N+]([O-])c1ccc2c(c1Oc1cccc(-n3ccc(F)n3)c1)CC[C@@H]2OP(=O)(N1CC1)N1CC1]

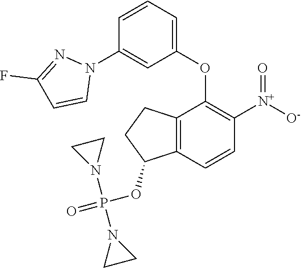 [SMILES: O=[N+]([O-])c1ccc2c(c1Oc1cccc(-n3ccc(F)n3)c1)CC[C@H]2OP(=O)(N1CC1)N1CC1]

TABLE A16
List of compounds

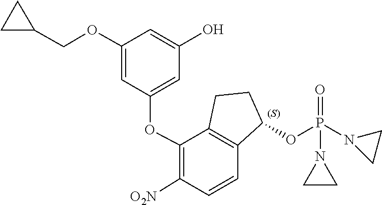

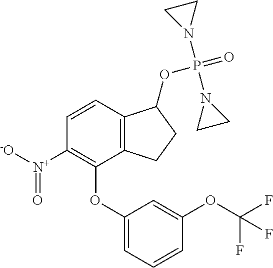

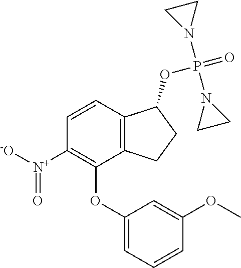

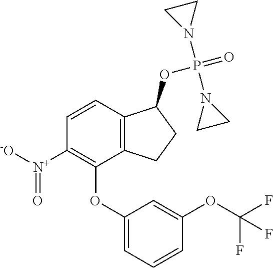

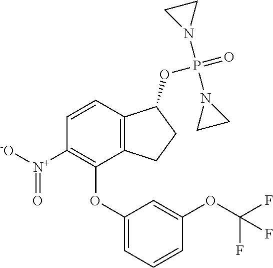

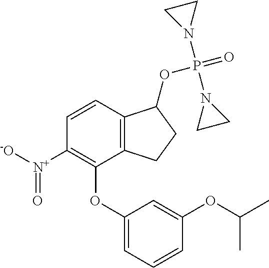

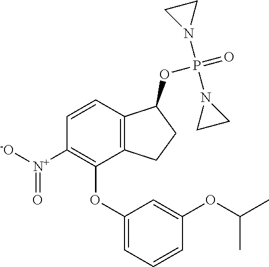

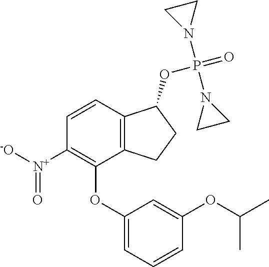

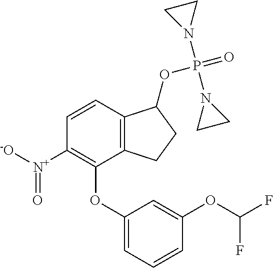

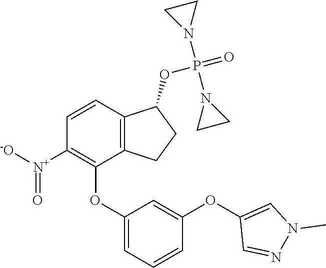

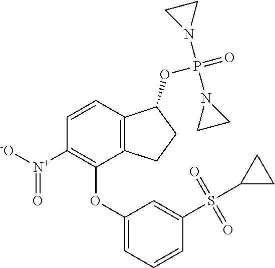

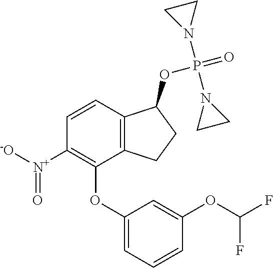

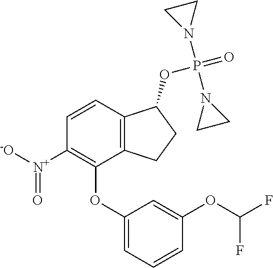

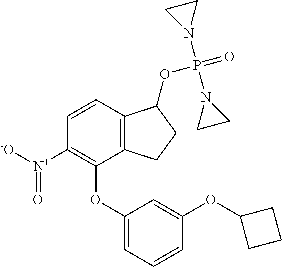

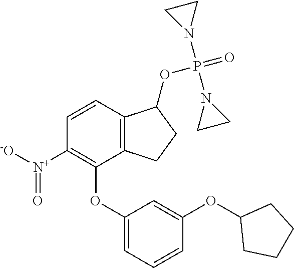

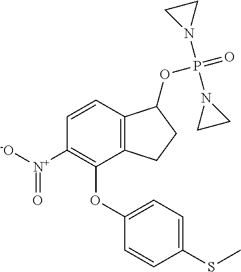

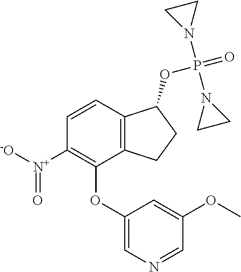

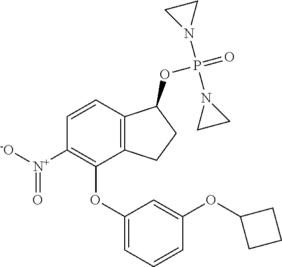

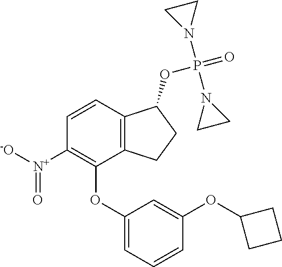

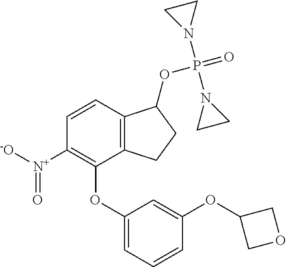

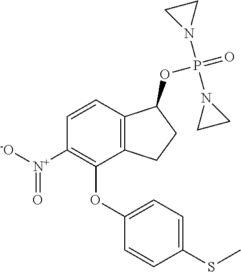

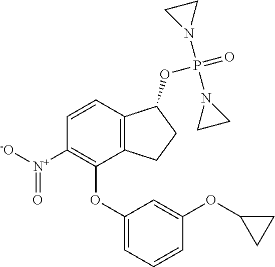

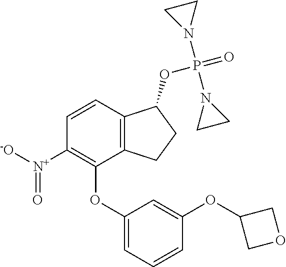

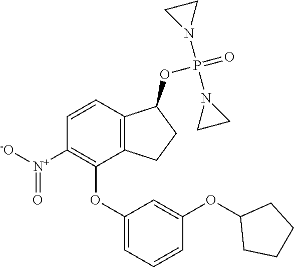

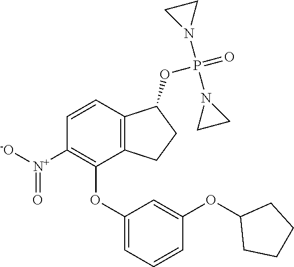

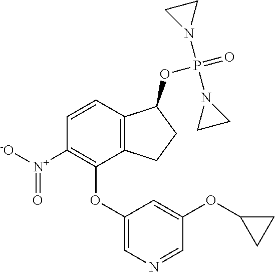

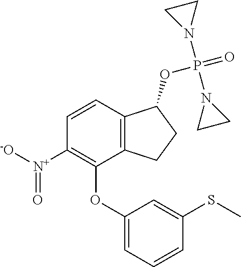

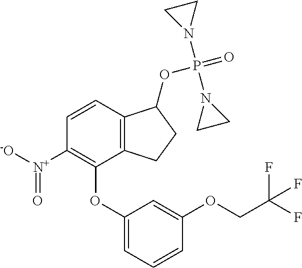

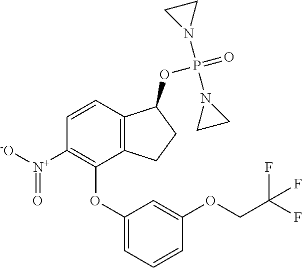

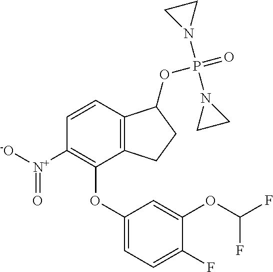

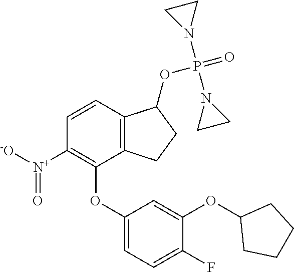

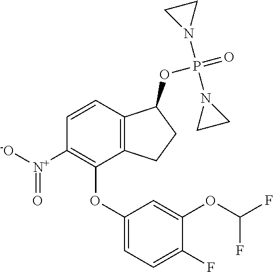

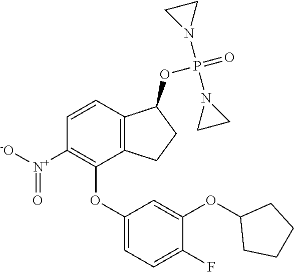

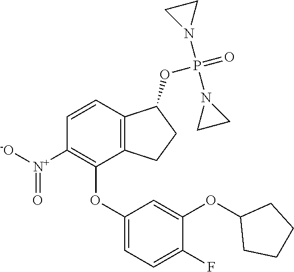

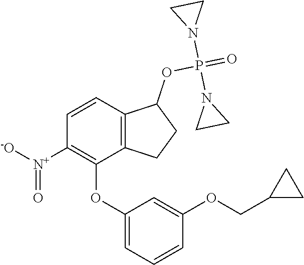

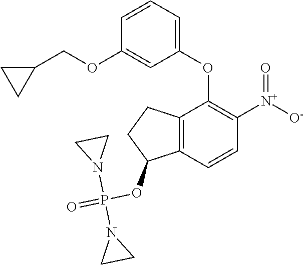 [SMILES: O=[N+]([O-])c1ccc2c(c1Oc1cccc(OCC3CC3)c1)CC[C@@H]2OP(=O)(N1CC1)N1CC1]

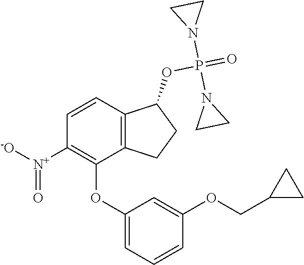

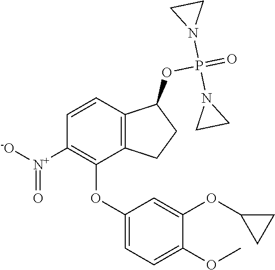

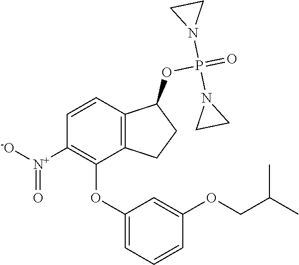

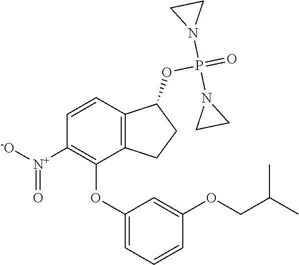

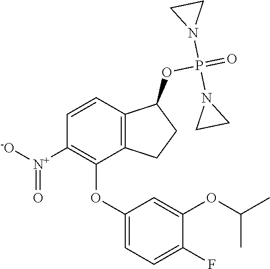

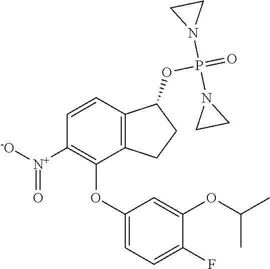

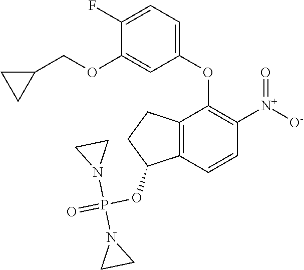 [SMILES: O=[N+]([O-])c1ccc2c(c1Oc1ccc(F)c(OCC3CC3)c1)CC[C@H]2OP(=O)(N1CC1)N1CC1]

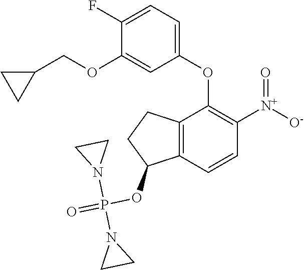 [SMILES: O=[N+]([O-])c1ccc2c(c1Oc1ccc(F)c(OCC3CC3)c1)CC[C@@H]2OP(=O)(N1CC1)N1CC1]

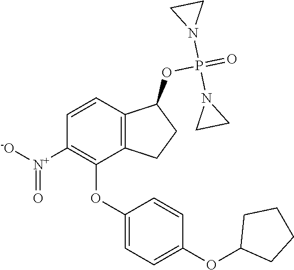

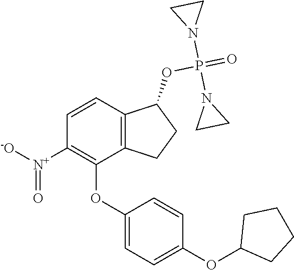

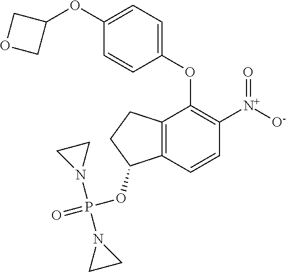 [SMILES: O=[N+]([O-])c1ccc2c(c1Oc1ccc(OC3COC3)cc1)CC[C@H]2OP(=O)(N1CC1)N1CC1]

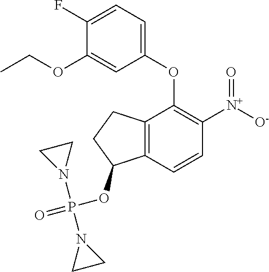 [SMILES: CCOc1cc(Oc2c([N+](=O)[O-])ccc3c2CC[C@@H]3OP(=O)(N2CC2)N2CC2)ccc1F]

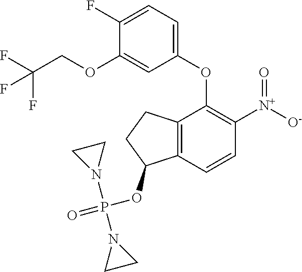 [SMILES: O=[N+]([O-])c1ccc2c(c1Oc1ccc(F)c(OCC(F)(F)F)c1)CC[C@@H]2OP(=O)(N1CC1)N1CC1]

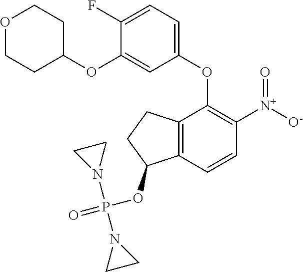 [SMILES: O=[N+]([O-])c1ccc2c(c1Oc1ccc(F)c(OC3CCOCC3)c1)CC[C@@H]2OP(=O)(N1CC1)N1CC1]

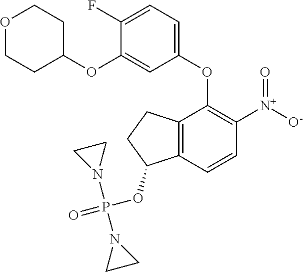 [SMILES: O=[N+]([O-])c1ccc2c(c1Oc1ccc(F)c(OC3CCOCC3)c1)CC[C@H]2OP(=O)(N1CC1)N1CC1]

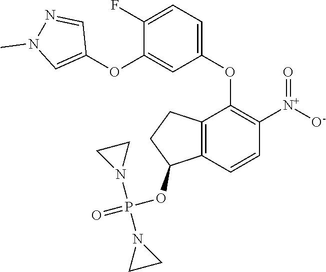 [SMILES: Cn1cc(Oc2cc(Oc3c([N+](=O)[O-])ccc4c3CC[C@@H]4OP(=O)(N3CC3)N3CC3)ccc2F)cn1]

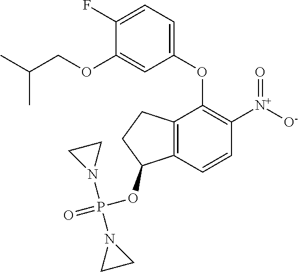 [SMILES: CC(C)COc1cc(Oc2c([N+](=O)[O-])ccc3c2CC[C@@H]3OP(=O)(N2CC2)N2CC2)ccc1F]

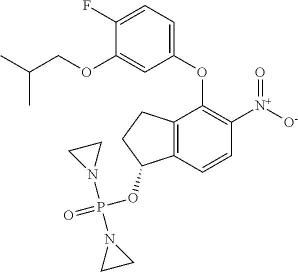 [SMILES: CC(C)COc1cc(Oc2c([N+](=O)[O-])ccc3c2CC[C@H]3OP(=O)(N2CC2)N2CC2)ccc1F]

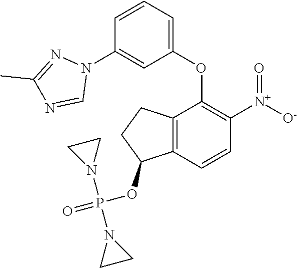 [SMILES: Cc1ncn(-c2cccc(Oc3c([N+](=O)[O-])ccc4c3CC[C@@H]4OP(=O)(N3CC3)N3CC3)c2)n1]

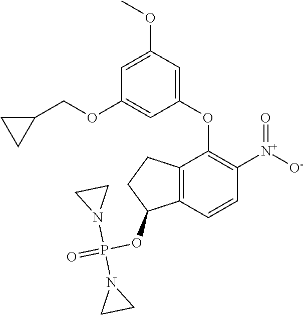 [SMILES: COc1cc(OCC2CC2)cc(Oc2c([N+](=O)[O-])ccc3c2CC[C@@H]3OP(=O)(N2CC2)N2CC2)c1]

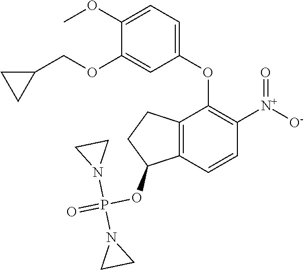 [SMILES: COc1ccc(Oc2c([N+](=O)[O-])ccc3c2CC[C@@H]3OP(=O)(N2CC2)N2CC2)cc1OCC1CC1]

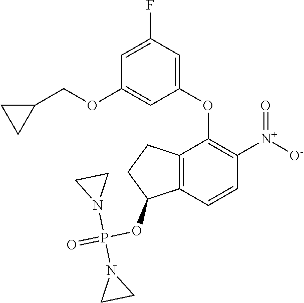 [SMILES: O=[N+]([O-])c1ccc2c(c1Oc1cc(F)cc(OCC3CC3)c1)CC[C@@H]2OP(=O)(N1CC1)N1CC1]

TABLE A17
List of compounds

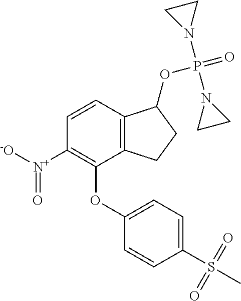

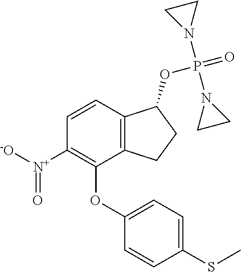

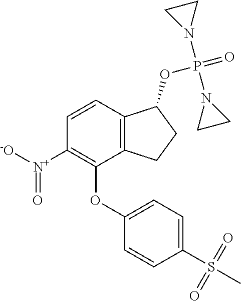

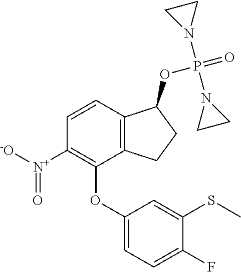

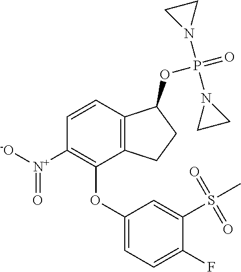

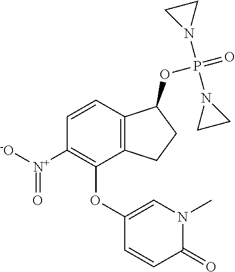

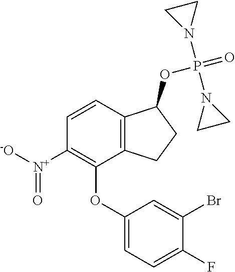

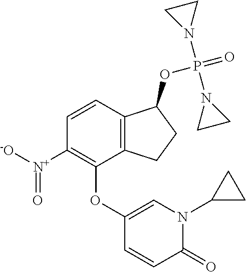

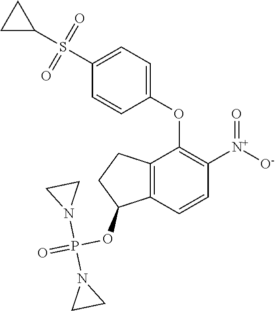 [SMILES: O=[N+]([O-])c1ccc2c(c1Oc1ccc(S(=O)(=O)C3CC3)cc1)CC[C@@H]2OP(=O)(N1CC1)N1CC1]

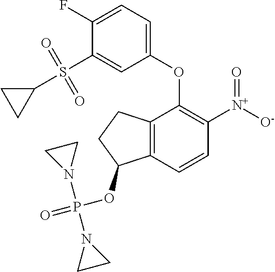 [SMILES: O=[N+]([O-])c1ccc2c(c1Oc1ccc(F)c(S(=O)(=O)C3CC3)c1)CC[C@@H]2OP(=O)(N1CC1)N1CC1]

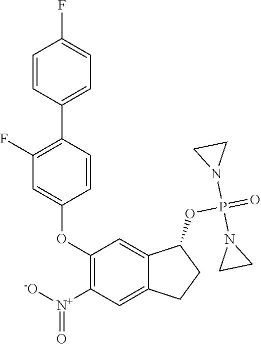

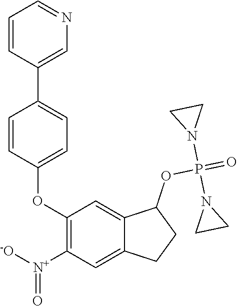

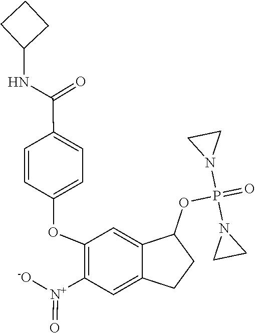

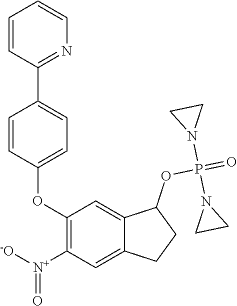

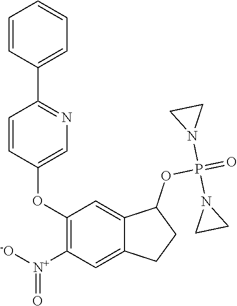

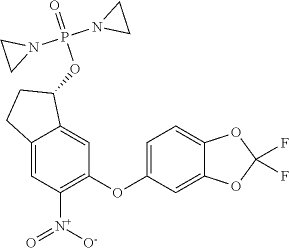 [SMILES: O=[N+]([O-])c1cc2c(cc1Oc1ccc3c(c1)OC(F)(F)O3)[C@@H](OP(=O)(N1CC1)N1CC1)CC2]

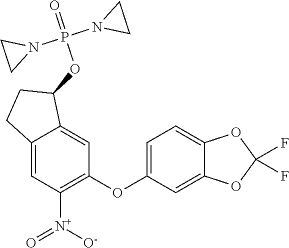 [SMILES: O=[N+]([O-])c1cc2c(cc1Oc1ccc3c(c1)OC(F)(F)O3)[C@H](OP(=O)(N1CC1)N1CC1)CC2]

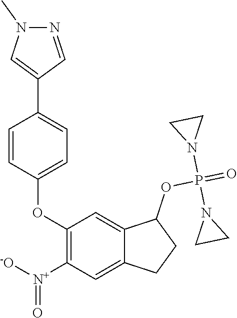

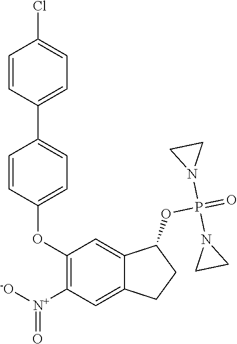

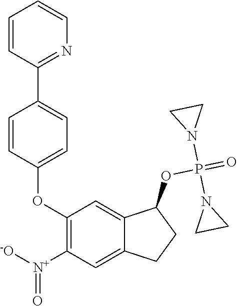

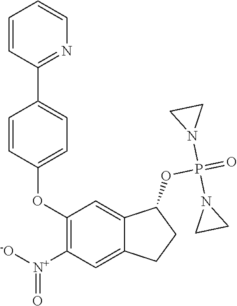

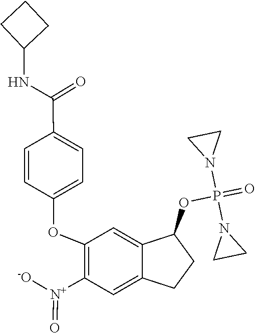

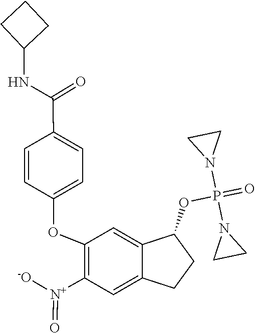

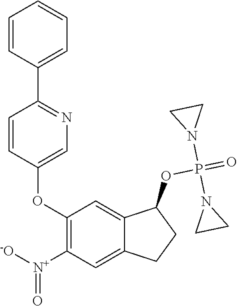

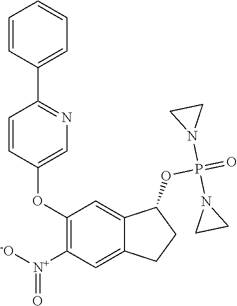

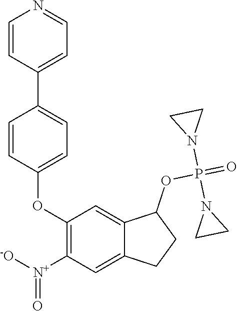

TABLE A18
List of compounds

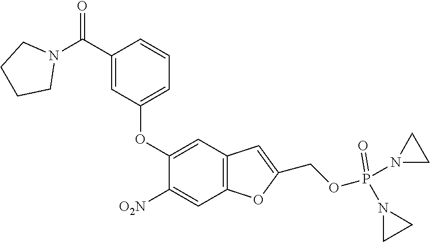 [SMILES: O=C(c1cccc(Oc2cc3cc(COP(=O)(N4CC4)N4CC4)oc3cc2[N+](=O)[O-])c1)N1CCCC1]

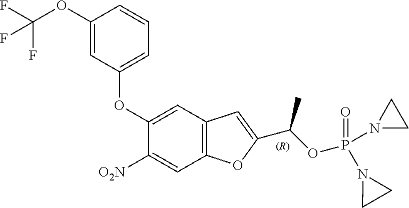

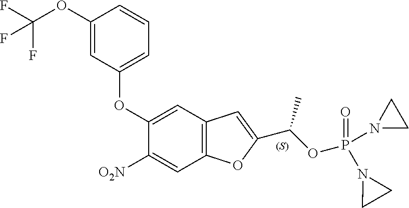

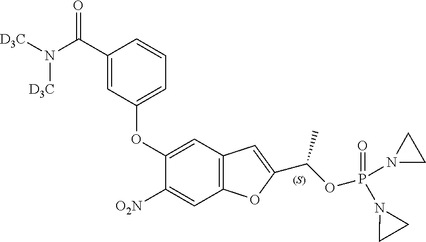

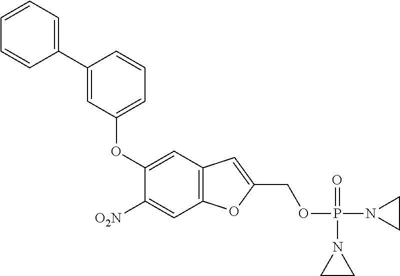 [SMILES: O=[N+]([O-])c1cc2oc(COP(=O)(N3CC3)N3CC3)cc2cc1Oc1cccc(-c2ccccc2)c1]

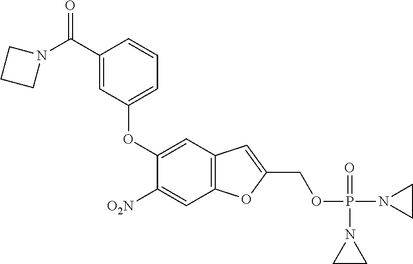 [SMILES: O=C(c1cccc(Oc2cc3cc(COP(=O)(N4CC4)N4CC4)oc3cc2[N+](=O)[O-])c1)N1CCC1]

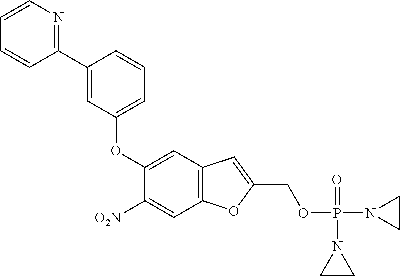 [SMILES: O=[N+]([O-])c1cc2oc(COP(=O)(N3CC3)N3CC3)cc2cc1Oc1cccc(-c2ccccn2)c1]

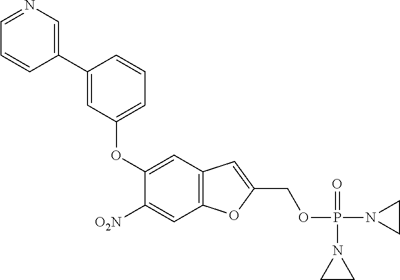 [SMILES: O=[N+]([O-])c1cc2oc(COP(=O)(N3CC3)N3CC3)cc2cc1Oc1cccc(-c2cccnc2)c1]

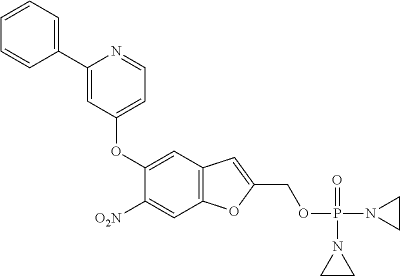 [SMILES: O=[N+]([O-])c1cc2oc(COP(=O)(N3CC3)N3CC3)cc2cc1Oc1ccnc(-c2ccccc2)c1]

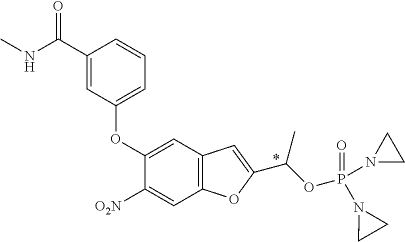 [SMILES: CNC(=O)c1cccc(Oc2cc3cc(C(C)OP(=O)(N4CC4)N4CC4)oc3cc2[N+](=O)[O-])c1]

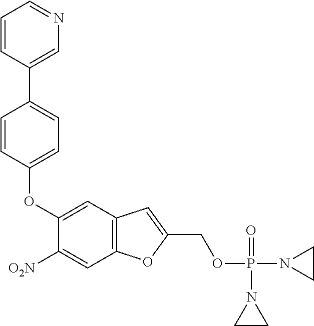 [SMILES: O=[N+]([O-])c1cc2oc(COP(=O)(N3CC3)N3CC3)cc2cc1Oc1ccc(-c2cccnc2)cc1]

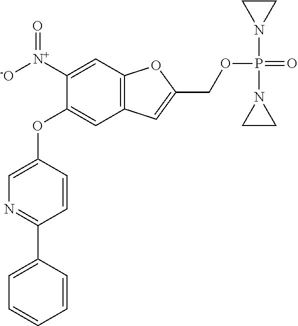

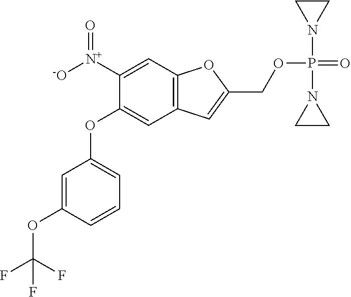

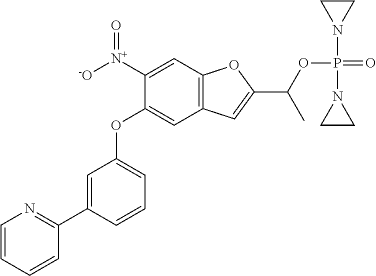

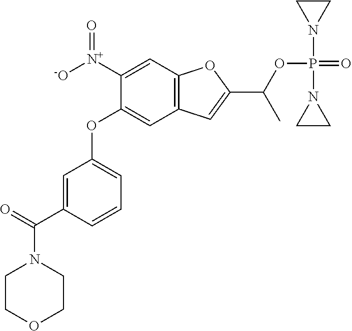

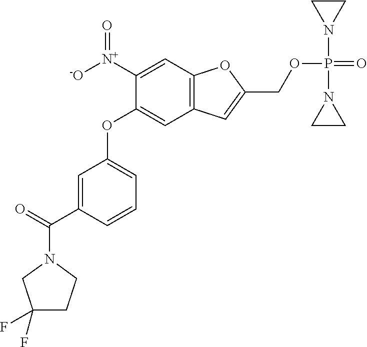

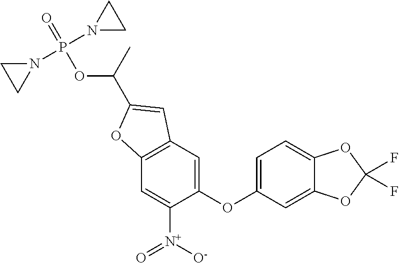 [SMILES: CC(OP(=O)(N1CC1)N1CC1)c1cc2cc(Oc3ccc4c(c3)OC(F)(F)O4)c([N+](=O)[O-])cc2o1]

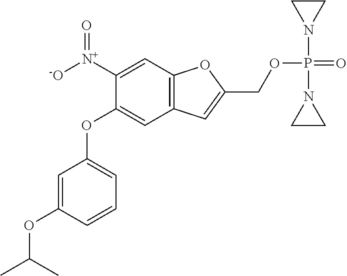

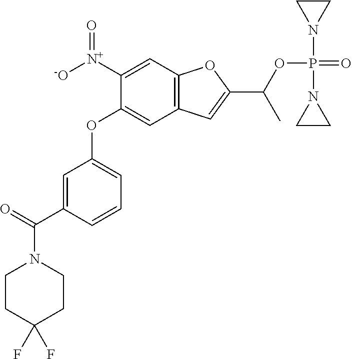

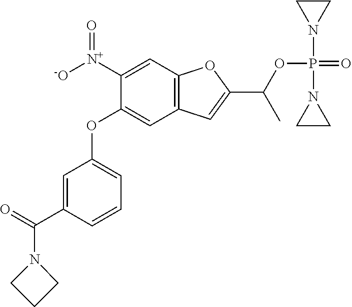

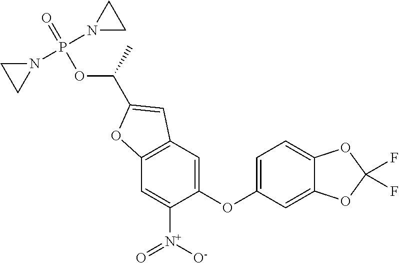 [SMILES: C[C@@H](OP(=O)(N1CC1)N1CC1)c1cc2cc(Oc3ccc4c(c3)OC(F)(F)O4)c([N+](=O)[O-])cc2o1]

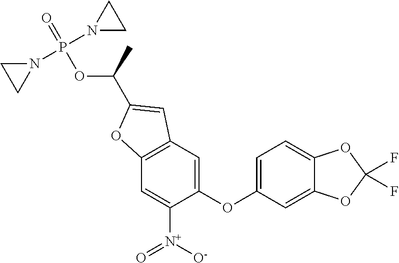 [SMILES: C[C@H](OP(=O)(N1CC1)N1CC1)c1cc2cc(Oc3ccc4c(c3)OC(F)(F)O4)c([N+](=O)[O-])cc2o1]

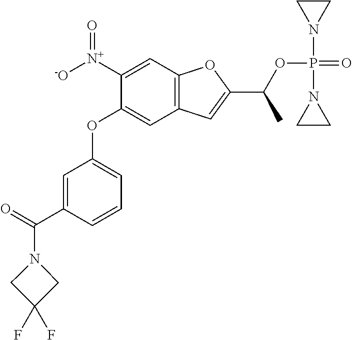

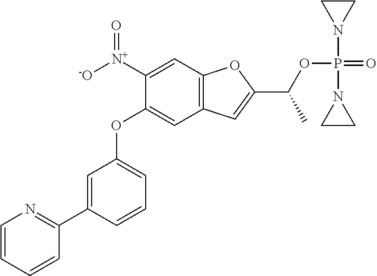

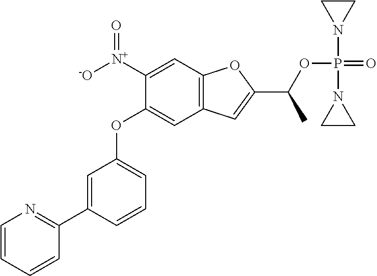

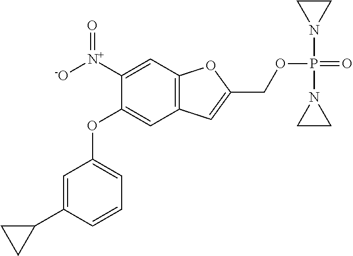

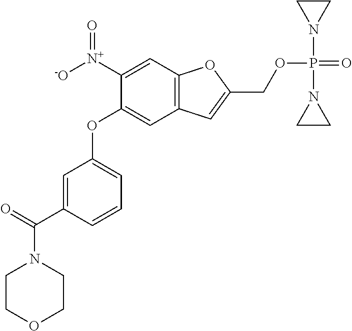

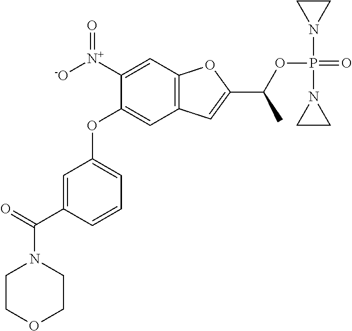

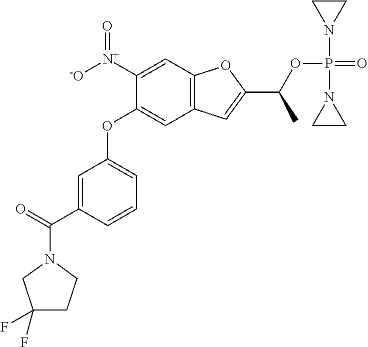

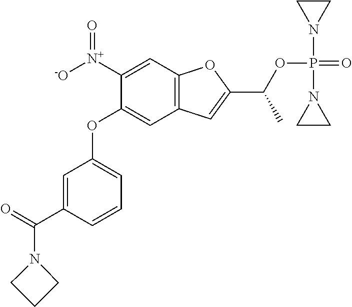

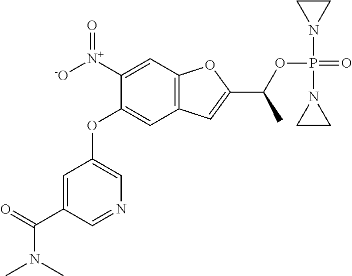

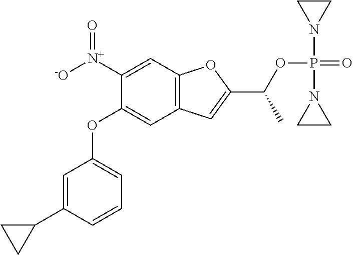

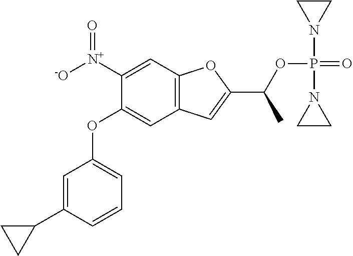

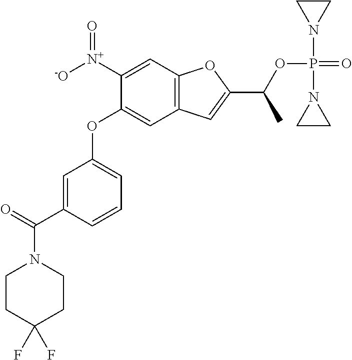

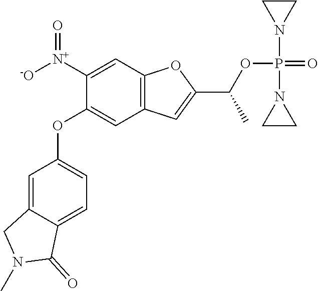

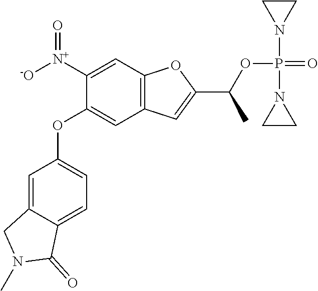

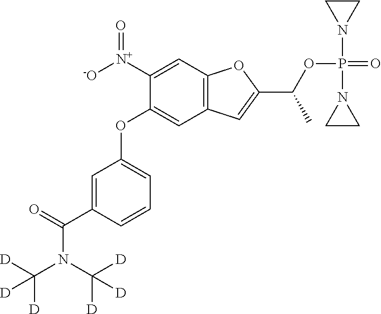

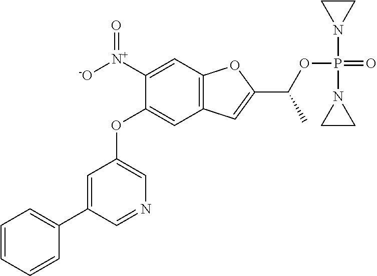

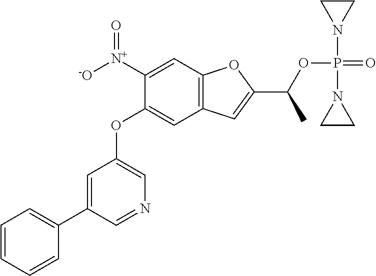

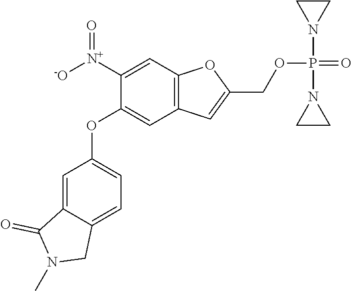

Compounds of Tables A1 to A18 can exist in various stereoisomeric forms, such as individual isomer, an individual enantiomer and/or diastereomer, as applicable, or a mixture of stereoisomers, including racemic mixtures and mixtures enriched in one or more stereoisomers. In some embodiments, when applicable, a compound shown Tables A1 to A18 can exist as an individual enantiomer substantially free (e.g., with less than 20%, less than 10%, less than 5%, less than 1%, by weight, by HPLC or SFC area, or both, or with a non-detectable amount) of the other enantiomer, such as having an enantiomeric excess of 60% or above, e.g., having 80% ee or above, 90% ee or above, 95% ee or above, 98% ee or above, or 99% ee or above. In some embodiments, when applicable, a compound shown Tables A1 to A18 can also exist as a mixture of stereoisomers in any ratio, such as a racemic mixture.

In some embodiments, to the extent applicable, the genus of compounds described herein also excludes any specifically known single compounds prior to this disclosure. In some embodiments, to the extent applicable, any sub-genus or species of compounds prior to this disclosure that are entirely within a genus of compounds described herein can also be excluded from such genus herein.

The compounds of the present disclosure can be readily synthesized by those skilled in the art in view of the present disclosure. Exemplified syntheses are also shown in the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., I-1, 1-2, 1-3, I-4, 1-5, I-6, I-7, I-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, 1-2, I-3, I-4, I-5, I-6, I-7, 1-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof, e.g., in a therapeutically effective amount. In any of the embodiments described herein, the pharmaceutical composition can comprise a therapeutically effective amount (e.g., for treating a cancer described herein) of a compound selected from any of Examples 1-569, or any of the specific compounds disclosed in Tables A1-A18 herein, or a pharmaceutically acceptable salt thereof.

The pharmaceutical composition herein can be formulated for delivery via any of the known routes of delivery, which include but not limited to administering orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present disclosure can be used alone, in combination with each other, or in combination with one or more additional therapeutic agents, e.g., in combination with an additional anticancer therapeutic agent, such as any of those approved on the market, such as those approved by the U.S. Food and Drug Administration or other similar government agencies.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as a cancer described herein, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of cancer, such as a cancer characterized as having abnormal AKR1C3 activity and/or overexpression of AKR1C3, for example, in an NRF2/KEAP1 mutated cancer.

In some embodiments, the present disclosure provides a method of treating or preventing a cancer in a subject in need thereof, the method comprising administering to the subject a therapeutically effective amount of one or more compounds of the present disclosure (e.g., Formula I (e.g., I-1, 1-2, 1-3, I-4, I-5, I-6, I-7, I-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof), or pharmaceutical compositions herein. Typically, the cancer is characterized as having abnormal AKR1C3 activity and/or overexpression of AKR1C3. In one embodiment, the cancer is liver cancer. In one embodiment, the cancer is non-small cell lung cancer. In one embodiment, the cancer is melanoma. In one embodiment, the cancer is prostate cancer, such as castration resistant prostate cancer.

In some embodiments, the present disclosure provides a method of treating or preventing a NRF2/KEAP1 mutated cancer in a subject in need thereof, the method comprising administering to the subject a therapeutically effective amount of one or more compounds of the present disclosure (e.g., Formula I (e.g., I-1, I-2, I-3, I-4, I-5, I-6, I-7, I-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, or a pharmaceutically acceptable salt thereof), or pharmaceutical compositions herein, wherein the NRF2/KAEP1 mutation results in an aberrant NRF2 activity, for example, leading to overexpression of AKR1C3. In one embodiment, the cancer is liver cancer. In one embodiment, the cancer is non-small cell lung cancer. In one embodiment, the cancer is melanoma. In one embodiment, the cancer is prostate cancer, such as castration resistant prostate cancer.

The types of cancer suitable to be treated with the methods herein are not particularly limited. For example, in some embodiments, the cancer is selected from the group consisting of a cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid; and acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyo sarcoma, renal cell tumor, topical skin lesion, veticulum cell sarcoma, and Wilm's tumor. In some embodiments, the cancer is liver cancer, non-small cell lung cancer, melanoma, renal cell carcinoma, or prostate cancer. In any of the embodiments described herein, the cancer can be characterized as having an abnormal AKR1C3 activity/AKR1C3 overexpression, which for example, may be caused by a NRF2/KEAP1 mutation. In one embodiment, the cancer is liver cancer. In one embodiment, the cancer is non-small cell lung cancer. In one embodiment, the cancer is melanoma. In one embodiment, the cancer is prostate cancer, such as castration resistant prostate cancer.

In some embodiments, the method comprises first determining the AKR1C3 reductase level of the cancer, such as by methods using an AKR1C3 antibody, and then administering a therapeutically effective amount of a compound of the present disclosure or a pharmaceutically acceptable composition provided herein to the subject if the AKR1C3 level is equal to or greater than a predetermined value, for example, if the AKR1C3 level is greater than the AKR1C3 level in a control healthy cell.

In some embodiments, the method comprises prior to administration, determining an intratumoral AKR1C3 reductase level in a sample isolated from the cancer subject and selecting the subject for the therapy if the AKR1C3 level is equal to or greater than a predetermined level, for example, if the AKR1C3 level is greater than the AKR1C3 level in a control healthy cell.

AKR1C3 levels can be measured following routine methods well known to the ordinarily skilled artisan. In some embodiments, provided herein is a kit comprising a means for isolating a sample from a patient and determining an intratumoral AKR1C3 reductase level of the cancer in the sample using an AKR1C3 antibody; and a means for determining whether a compound of the present disclosure or composition provided herein should be administered. Methods of determining the therapeutically effective amount, appropriate mode of administration of the compounds and compositions provided herein will be apparent to the ordinarily skilled artisan in view of the present disclosure and based on other methods known to them.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intravenous injection.

Compounds of the present disclosure can be used as a monotherapy or in a combination therapy. In some embodiments according to the methods described herein, one or more compounds of the present disclosure can be administered as the only active ingredient(s). In some embodiments according to the methods described herein, one or more compounds of the present disclosure can also be co-administered with an additional therapeutic agent, either concurrently or sequentially in any order, to the subject in need thereof. The additional therapeutic agent can typically be an additional anticancer therapeutic agent, such as any of those approved on the market, such as those approved by the U.S. Food and Drug Administration or other similar government agencies.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I, II, or III, or a subformula thereof, as applicable, are independently selected. Non-limiting useful groups for the variables in compounds of Formula I, II, or III, or a subformula thereof, as applicable, include any of the respective groups, individually or in any combination, as shown in the Examples or in the specific compounds described in Tables A1 to A18 herein. Using variable R³ as an example, in some embodiments, compounds of Formula I, II, or III can include a R³ group according to any of the R³ groups shown in the Examples or in the specific compounds described in Tables A1-A18 herein, without regard to the other variables shown in the specific compounds. In some embodiments, compounds of Formula I, II, or III can include a R³ group according to any of the R³ groups shown in the Examples or in the specific compounds described in Tables A1-A18 herein in combination at least one other variable (e.g, X) according to the Examples or the specific compounds described in Tables A1-A18 herein, wherein the R³ and at least one other variable can derive from the same compound or a different compound. Any of such combinations are contemplated and within the scope of the present disclosure.

The described embodiments of the present disclosure can be combined. Such combination is contemplated and within the scope of the present disclosure. For example, it is contemplated that the definition(s) of any one or more of R¹, R², R³, R₄, R⁵, R⁶, R^{a}, R^{b}, n1, n2, and X of Formula I (e.g., Formula I-1, I-2, I-3, I-4, I-5, I-6, 1-7, I-8, I-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8) can be combined with the definition of any one or more of the other(s) of R¹, R², R³, R₄, R⁵, R⁶, R^{a}, R^{b}, n1, n2, and X, as applicable, and the resulted compounds from the combination are within the scope of the present disclosure.

The symbol, [symbol] , displayed perpendicular to (or otherwise crossing) a bond, such as

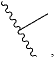

indicates the point at which the displayed moiety is attached to the remainder of the molecule. It should be noted that the immediately connected group or groups may be shown beyond the symbol, [symbol] , to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, 1N 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s), for example, with respect to that particular chiral center or axial chirality, the compound can have an enantiomeric excess (“ee”) of greater than 60%, such as 80% ee or greater, 90% ee or greater, 95% ee or greater, 98% ee or greater, 99% ee or greater, etc. The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures. For example, those skilled in the art would understand Formula I includes the corresponding compound in any stereoisomeric forms and mixtures of various isomers.

Unless otherwise contradictory from context, when the stereochemical configuration for a chiral center in a compound prepared in the examples is drawn stereo specifically (e.g., with widget and/or dash bonds), either without additional designation or being designated “R” (or “(R)”) or “S” (or “(S)”), it means the two enantiomers at that chiral center were separated and absolute stereochemistry was known, or only one enantiomer was obtained and absolute stereochemistry was known. Unless otherwise contradictory from context, when no stereochemistry is specifically drawn for a chiral center in a compound prepared in the examples (e.g., with a straight bond) but “*” is used to indicate the chiral center, it means the absolute stereochemistry is undetermined although the compound itself has been isolated as a single stereoisomer and is enantiomerically pure or enriched. Unless otherwise contradictory from context, when no stereochemistry is specifically drawn for a chiral center in a compound prepared in the examples (e.g., with a straight bond) and no “*” is used to indicate the chiral center, it means the compound is a racemic mixture at that chiral center.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃-4, C₄-6, C₄-5, and C₅-6.

As used herein, the term “compound(s) of the present disclosure” refers to any of the compounds described herein according to Formula I (e.g., I-1, 1-2, 1-3, 1-4, 1-5, I-6, 1-7, I-8, 1-9, I-10, I-11, I-12, I-13, I-1-A, I-1-B, I-1-B-E1, I-1-B-E2, I-5-B-E1, I-5-B-E2, I-7-A, I-7-B, I-7-B-E1, I-7-B-E2, I-11-A, I-11-B, I-11-B-E1, I-11-B-E2, I-13-A, I-13-B, I-13-B-E1, I-13-B-E2, I-13-B-E3, I-13-B-E4, IV-1, IV-2, IV-3, IV-4, IV-5, IV-6, IV-7, or IV-8), Formula II (e.g., II-1, II-2, II-3, II-4, II-3-A, II-4-A, II-3-B, II-4-B, II-3-A-E1, II-3-A-E2, II-4-A-E1, II-4-A-E2, II-4-A-E3, II-4-A-E4, II-4-B-E1, II-4-B-E2, or V-1), Formula III (e.g., III-1, III-2, III-3, or III-4), any of Examples 1-569, or any of the specific compounds disclosed in Tables A1-A18 herein, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). To be clear, compounds of Examples 1-569 refer to the compounds in the Examples section labeled with an integer only, such as 1, 2, etc. up to 569. See e.g., Illustration 1-51 and characterization tables herein. Exemplified synthesis and characterizations of Examples 1-569 are shown in the Examples section. Detailed exemplified procedures were shown in the Illustration examples, e.g., 1-51. Hydrates and solvates of the compounds of the present disclosure are considered compositions of the present disclosure, wherein the compound(s) is in association with water or solvent, respectively. In some embodiments, the compound of the present disclosure can be any of those described in Embodiments A1 to A14 or B1 to B14.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I . Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein Ral is an alkyl.

As used herein, the term “cycloalkoxy” as used by itself or as part of another group refers to a radical of the formula OR^{a1}, wherein Ral is a cycloalkyl.

As used herein, the term “haloalkyl” as used by itself or as part of another group refers to an alkyl substituted with one or more fluorine, chlorine, bromine and/or iodine atoms. In some embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In some embodiments, the cycloalkyl is a monocyclic ring. As used herein, the term “cycloalkylene” as used by itself or as part of another group refers to a divalent radical derived from a cycloalkyl group, for example,

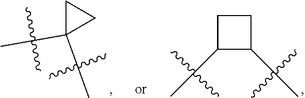 [SMILES: *C1(*)CC1 ; *C1CCC1*]

etc.

“Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings, and the point of attachment can be on any ring. As used herein, the term “heterocyclylene” as used by itself or as part of another group refers to a divalent radical derived from the heterocyclyl group defined herein. The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4 membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆-14 aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). As used herein, the term “arylene” as used by itself or as part of another group refers to a divalent radical derived from the aryl group defined herein.

“Aralkyl” as used by itself or as part of another group refers to an alkyl substituted with one or more aryl groups, in one embodiment, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, in one embodiment, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, in one embodiment, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Unless otherwise indicated, when the number of substituent(s) for a “substituted” group is not specified, it means the group is substituted with one or more substituents, as permitted by valency. For example, unless otherwise indicated, a C₁₋₄ alkyl optionally substituted with F refers to a C₁₋₄ alkyl optionally substituted with one or more F, including, e.g., CF₃. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable, each of which can be optionally isotopically labeled, such as deuterated. Two of the optional substituents can join to form a ring structure, such as an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N (except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, −C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(—N—CN)—NH₂, C(═NH)—NH₂, —C(—NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₅)alkyl groups, (C₂-C₈)alkenyl groups, (C₂-C₅)alkynyl groups, (C₃-C₁₀) cycloalkyl groups, halogen (F, Cl, Br or I), halogenated (C₁-C₅)alkyl groups (for example but not limited to —CF₃), —O—(C₁-C₅)alkyl groups, —OH, —S—(C₁-C₈)alkyl groups, —SH, —NH(C₁-C₈)alkyl groups, —N((C₁-C₅)alkyl)₂ groups, —NH₂, —C(O)NH₂, —C(O)NH(C₁-C₅)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₅)alkyl)C(O)H, —N((C₁-C₈)alkyl)C(O)(C₁-C₈)alkyl groups, —NHC(O)NH₂, —NHC(O)NH(C₁-C₅)alkyl groups, —N((C₁-C₈)alkyl)C(O)NH₂ groups, —NHC(O)N((C₁-C₅)alkyl)₂ groups, —N((C₁-C₅)alkyl)C(O)N((C₁-C₈)alkyl)₂ groups, —N((C₁-C₈)alkyl)C(O)NH((C₁-C₅)alkyl), —C(O)H, —C(O)(C₁-C₅)alkyl groups, —CN, —NO₂, —S(O)(C₁-C₈)alkyl groups, —S(O)₂(C₁-C₅)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₅)alkyl groups, —N((C₁-C₈)alkyl)S(O)₂(C₁-C₈)alkyl groups, —(C₁-C₈)alkyl-O—(C₁-C₈)alkyl groups, —O—(C₁-C₅)alkyl-O—(C₁-C₈)alkyl groups, —C(O)OH, —C(O)O(C₁-C₅)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₅)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆-14 aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆-14 aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from deuterium, F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from deuterium, F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C¹⁻⁴ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, in one embodiment a mammal, in another embodiment a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc, which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Exemplary embodiments of steps for performing the synthesis of products described herein are described in greater detail infra. Some of the Examples discussed herein can be prepared by separating from the corresponding racemic mixtures. As would be understood by a person of ordinary skill in the art, the compounds described in the Examples section immediately prior to the chiral separation step, e.g., by supercritical fluid chromatography (SFC), exist in racemic and/or stereoisomeric mixture forms, the bolded but not wedged bonds are used in the chemical structure drawings to indicate relative stereochemistry. It should be understood that the enantiomeric excesses (“ee”) reported for these examples are only representative from the exemplified procedures herein and not limiting; those skilled in the art would understand that such enantiomers with a different ee, such as a higher ee, can be obtained in view of the present disclosure.

Table of Abbreviations

| | |
|---|---|
| ACN | acetonitrile |
| aq. | aqueous |
| B₂pin₂ | bis(pinacolato)diboron |
| Boc | tert-butyloxycarbonyl |
| Cbz | carbonylbenzyloxy |
| CDI | carbonyldiimidazole |
| Cpd | Compound |
| DCE | 1,2-dichloroethane |
| DCM | dichloromethane |
| DEPBT | (3-(diethoxyphosphoryloxy)-1,2,3-benzotriazin-4(3H)-one) |
| DEAD | diethyl azodicarboxylate |
| DIAD | diisopropyl azodicarboxylate |
| DIPEA | N,N-diisopropylethylamine |
| DMAP | N,N-dimethylpyridin-4-amine |
| DMC | dimethyl carbonate |
| DMEDA | N,N′-dimethylethylenediamine |
| DMF | dimethylformamide |
| DMP | 1,1-bis(acetyloxy)-3-oxo-3H-1λ⁵,2-benziodaoxol-1-yl acetate |
| DMSO | dimethyl sulfoxide |
| DMT | dimercaptotriazine |
| DPPA | {[azido(phenoxy)phosphoryl]oxy}benzene |
| EA | Ethyl acetate |
| EDC | 1-ethyl-3-(3-dimethylaminopropyl)carbodiimide |
| eq | equivalent or equivalents |
| FA | formic acid |
| h | hour or hours |
| HATU | 1-[bis(dimethylamino)methylene]-1H-1,2,3-triazolo[4,5-b]pyridinium-3- |
| | oxide hexafluorophosphate |
| HOAt | 1-hydroxy-7-azabenzotriazole |
| HOBt | 1-hydroxybenzotriazole |
| HPLC | high pressure liquid chromatography |
| LCMS | liquid chromatography mass spectrometry |
| LDA | lithium diisopropylamide |
| mCPBA | meta-chloroperbenzoic acid |
| min | minute or minutes |
| MTBE | methyl tert-butyl ether |
| NBS | N-bromosuccinimide |
| NCS | 1-chloropyrrolidine-2,5-dione |
| NMI | N-methylimidazole |
| NMR | nuclear magnetic resonance |
| Pbf | 2,2,4,6,7-pentamethyl-2,3-dihydrobenzofuran-5-yl)sulfonyl |
| Pd₂(dba)₃ | tris(dibenzylideneacetone)dipalladium(0) |
| Pd(dppf)Cl₂ | [1,1′-bis(diphenylphosphino)ferrocene]dichloropalladium(II) |
| PE/Pet Ether | petroleum ether |
| Py | pyridine |
| PyBOP | benzotriazol-1-yloxytripyrrolidinophosphonium hexafluorophosphate |
| RT | room temperature |
| T3P | propanephosphonic anhydride |
| TBAF | Tetrabutylammonium fluoride |
| TCFH | N,N,N′,N′-tetramethylchloroformamidinium hexafluorophosphate |
| TEA | triethylamine |
| TFA | trifluoroacetic acid |
| THF | tetrahydrofuran |
| TLC | thin layer chromatography |
| TMSCl | Trimethylsilyl chloride |
| TMSI | Trimethylsilyl iodide |
| TFA | trifluoroacetic acid |
| XantPhos | 4,5-bis(diphenylphosphino)-9,9-dimethylxanthene |
| XantPhos Pd | (6-diphenylphosphanyl-10H-phenoxazin-4-yl)- |
| G4 | diphenylphosphane; methanesulfonic acid; N-methyl-2- |
| | phenylaniline; palladium |
| XPhos | 2-dicyclohexylphosphino-2′,4′,6′-triisopropylbiphenyl |
| XPhos Pd G3 | dicyclohexyl-[2-[2,4,6-tri(propan-2- |
| | yl)phenyl]phenyl]phosphane; methanesulfonate; palladium; 2-phenylaniline |

### Analytical Instrumentation and Purification

NMR Instrument Details: Varian 400 MHz, Probe-1: Auto XID, Probe 2: ATB.

LCMS Instrument Details: Shimadzu LCMS-2010EV system coupled to SPD-M20A PDA and ELS detectors. Softa model 400.

### Synthesis of 3-hydroxy-N,N-dimethylbenzamide (Int. 1)

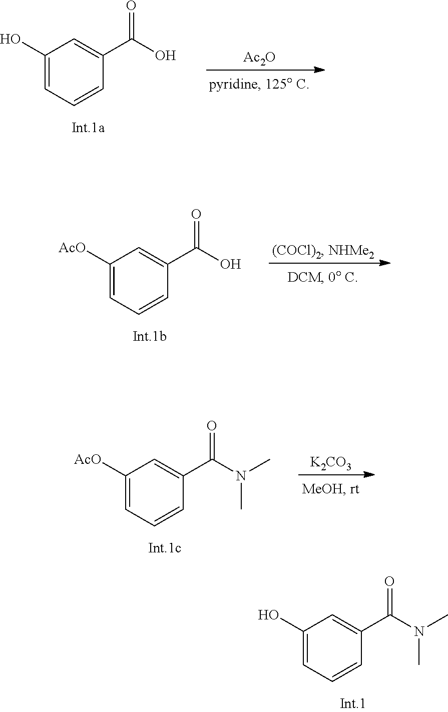

3-acetoxybenzoic acid

To a solution of 3-hydroxybenzoic acid (10.0 g, 0.14 mol, 1.0 eq.) in pyridine (100 mL) was added acetic anhydride (50 mL) and the mixture was stirred at 125° C. for 2 hrs under nitrogen. After completion, the reaction mixture was cooled down in an ice bath, then neutralized carefully with aqueous NaHCO₃ (30 mL, sat.), acidified with 12 N HCl until the pH was adjusted to pH=2. The suspension was filtered, the filter cake was washed with H₂O (100 mL) and dried in vacuum to give 3-acetoxybenzoic acid (12.0 g, 66.7 mmol, 92%) as a brown solid. LC-MS (ESI): m/z 181 [M+H]+. ¹H NMR (400 MHz, DMSO-d₆) δ 8.59-8.55 (m, 1H), 7.86-7.81 (m, 1H), 7.68-7.64 (m, 1H), 7.55 (t, J=7.9 Hz, 1H), 7.41-7.37 (m, 1H), 2.29 (s, 3H).

3-(dimethylcarbamoyl)phenyl acetate

To a stirred suspension of 3-acetoxybenzoic acid (6.0 g, 33.3 mmol, 1.0 eq.) in dry DCM (60 mL) was added oxalic dichloride (14.3 mL, 166.5 mmol, 5.0 eq.) drop-wisely at 0° C. under nitrogen. After stirring at 0° C. for 4 hrs, a solution of dimethylamine (33.3 mL, 66.6 mmol, 2M in THF, 2.0 eq.) was added drop-wisely below 0° C. and the mixture was stirred for another 20 hrs. After completion, the resulting mixture was poured into ice HCl aqueous solution (20 mL, 1 M), extracted with DCM (50 mL×2). The combined organic phases were dried over anhydrous Na₂SO₄, filtered and concentrated under reduced pressure. The residue was purified by Biotage® C18 column chromatography to give 3-(dimethylcarbamoyl)phenyl acetate (6.4 g, 31.0 mmol, 99%) as a yellow solid. LC-MS (ESI): m/z 208 [M+H]⁺.

3-hydroxy-N,N-dimethylbenzamide

To a solution of 3-(dimethylcarbamoyl)phenyl acetate (6.4 g, 31.0 mmol, 1.0 eq.) in MeOH (70 mL) was added K₂CO₃ (4.5 g, 32.6 mmol, 1.05 eq.) under nitrogen. The reaction mixture was stirred at room temperature for 18 hrs. The suspension was filtered and the filter cake was washed with MeOH (20 mL). The combined filtrates were concentrated to give 3-hydroxy-N,N-dimethylbenzamide (5.3 g, 32.1 mmol, 98%) as a colorless oil, which was used in next step directly without further purification. LC-MS (ESI): m/z 166 [M+H]+.

### Synthesis of 2-cyclopropyl-6-hydroxyisoindolin-1-one (Int.2, top piece)

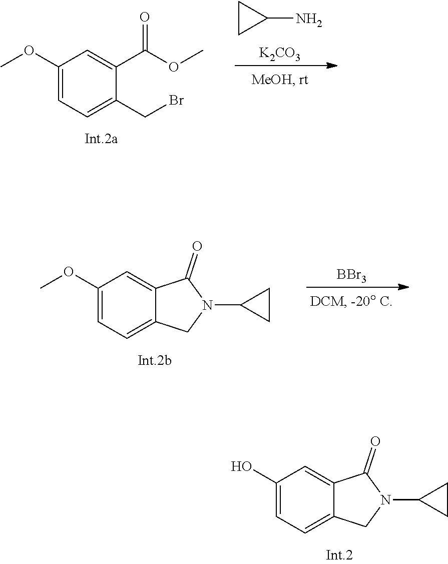

2-cyclopropyl-6-methoxyisoindolin-1-one

To a solution of methyl 2-(bromomethyl)-5-methoxybenzoate (300 mg, 1.16 mmol, 1.0 eq.) and cyclopropanamine (72.72 mg, 1.27 mmol, 1.1 eq.) in MeOH (5 mL) was added dipotassium carbonate (400.07 mg, 2.90 mmol, 2.5 eq.) at room temperature. The resulting mixture was stirred at room temperature for 18 hrs. After completion, the reaction mixture was concentrated under reduced pressure. The residue was purified by flash column chromatography on silica gel to give 2-cyclopropyl-6-methoxyisoindolin-1-one (200 mg, 0.98 mmol, 85%) as a white solid. LC-MS (ESI): m/z 204.2 [M+H]⁺.

2-cyclopropyl-6-hydroxyisoindolin-1-one

To a solution of 2-cyclopropyl-6-methoxyisoindolin-1-one (100 mg, 0.49 mmol) in DCM (5 mL) was added drop-wisely tribromoborane (0.07 mL, 0.74 mmol, 1.5 eq.) in DCM (1 mL) at −20° C. The reaction mixture was stirred at −20° C. for 2 hrs under nitrogen atmosphere. After completion, the reaction mixture was poured into ice-water (5 mL) at 0° C., then extracted with DCM (10 mL×3). The combined organic layers were dried over anhydrous Na₂SO₄, and concentrated under reduce pressure to give 2-cyclopropyl-6-hydroxyisoindolin-1-one (60 mg, 0.32 mmol, 64%) as a white solid. LC-MS (ESI): m/z 190.1 [M+H]⁺.

### Synthesis of 6-hydroxy-2-methylisoindolin-1-one (Int.3)

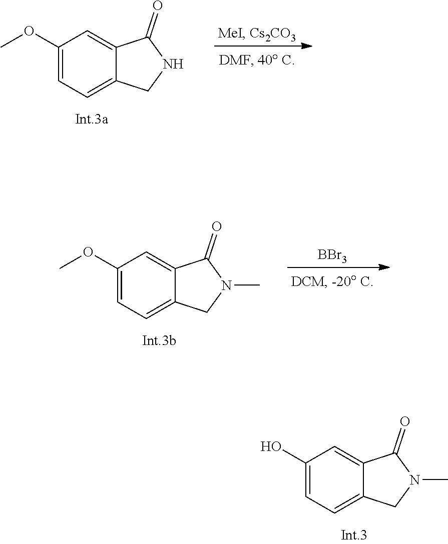

6-methoxy-2-methylisoindolin-1-one

To a solution 6-methoxyisoindolin-1-one (200 mg, 1.23 mmol, 1.0 eq.) in DMF (5 mL) were added CS₂CO₃ (798.68 mg, 2.45 mmol, 2.0 eq.) and iodomethane (0.15 mL, 2.40 mmol, 2.0 eq.) at 25° C. The reaction mixture was heated to 40° C. and stirred for 2 hrs under nitrogen atmosphere. After completion, the reaction mixture was cooled to room temperature, poured into water (10 mL) and extracted with EtOAc (5 mL×2). The combined organic layers were washed with brine (5 mL), dried over anhydrous Na₂SO₄, filtered and concentrated under reduced pressure to give 6-methoxy-2-methylisoindolin-1-one (180 mg, 1.02 mmol, 83%) as a white solid. LC-MS (ESI): m/z=178 [M+H]⁺.

6-hydroxy-2-methylisoindolin-1-one

To a solution of 6-methoxy-2-methylisoindolin-1-one (180 mg, 1.02 mmol, 1.0 eq.) in DCM (5 mL) was added drop-wisely tribromoborane (0.19 mL, 2.03 mmol, 2.0 eq.) at −20° C. The reaction mixture was stirred at −20° C. for 2 hrs under nitrogen atmosphere. After completion, the reaction mixture was poured into ice-water (5 mL) at 0° C., then extracted with DCM (5 mL×2). The combined organic layers were washed with brine (5 mL), dried over anhydrous Na₂SO₄, and concentrated under reduce pressure to give 6-hydroxy-2-methylisoindolin-1-one (160 mg, 0.981 mmol, 97%) as a yellow solid, which was used in next step directly without further purification. LC-MS (ESI): m/z 164 [M+H]⁺.

### Synthesis of 4-fluoro-5-nitro-2,3-dihydro-1H-inden-1-ol (Int.4)

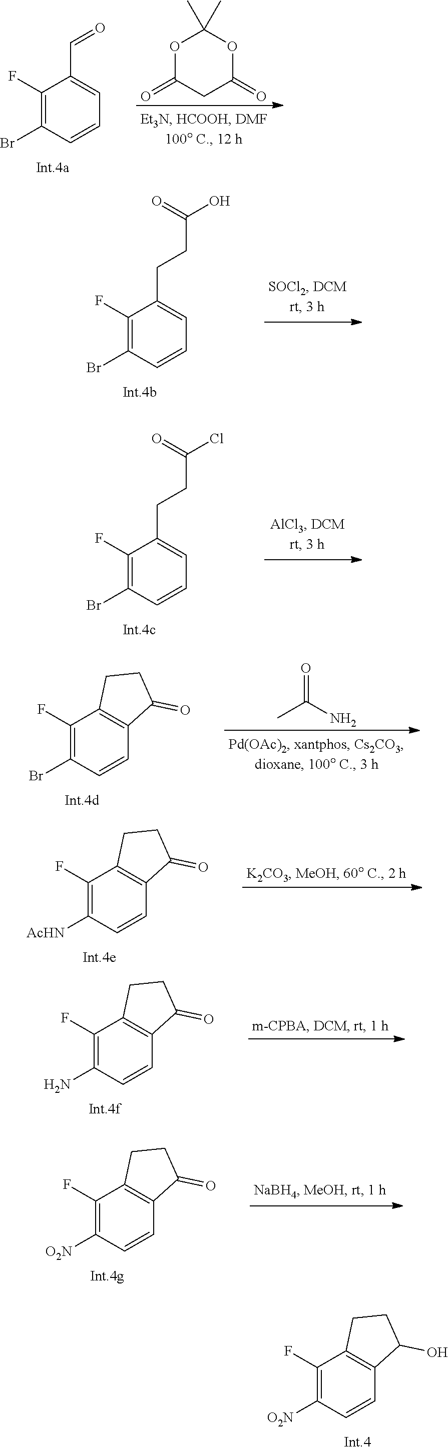

3-(3-bromo-2-fluorophenyl)propanoic acid

Into Et₃N (38.7 g, 382 mmol, 1.30 equiv) was added HCOOH (40.6 g, 882 mmol, 3.00 equiv) dropwise at 0° C. The mixture was stirred for 15 min at room temperature. To the above mixture was added DMF (500 mL), 3-bromo-2-fluorobenzaldehyde (59.7 g, 294 mmol, 1.00 equiv) and meldrum's acid (42.4 g, 294 mmol, 1.00 equiv) at room temperature. The resulting mixture was stirred for additional 12 h at 100° C. The mixture was allowed to cool down to room temperature and poured into conc. HCl/ice water (1/10 v/v) (3.0 L). The precipitated solids were collected by filtration and washed with water (3×300 mL). The solid was dried under reduced pressure. This resulted in 3-(3-bromo-2-fluorophenyl) propanoic acid (64.5 g, crude) as an off-white solid. LC-MS (ESI): m/z 244.80, 246.80 [M−H]⁻

3-(3-bromo-2-fluorophenyl)propanoyl chloride

To a stirred solution of 3-(3-bromo-2-fluorophenyl) propanoic acid (80.0 g, 324 mmol, 1.00 equiv) and DMF (0.50 mL, 6.48 mmol, 0.02 equiv) in DCM (500 mL) was added SOCl₂ (116 g, 972 mmol, 3.00 equiv) dropwise at 0° C. The resulting mixture was stirred for 3 h at room temperature. The resulting mixture was concentrated under reduced pressure To afford 3-(3-bromo-2-fluorophenyl) propanoyl chloride (82.0 g, crude) as a yellow oil.

5-bromo-4-fluoro-2,3-dihydroinden-1-one

To a stirred solution of 3-(3-bromo-2-fluorophenyl) propanoyl chloride (82.0 g, 308.8 mmol, 1.00 equiv) in DCM (500.0 mL) was added AlCl₃ (61.8 g, 463.3 mmol, 1.50 equiv) in portions at 0° C. The resulting mixture was stirred for 15 min at 0° C. and then warm up to room temperature to continue to stir for 3 h under nitrogen atmosphere. The resulting mixture was poured into ice water (1000 mL). The resulting mixture was filtered, the filter cake was washed with DCM (3×100 mL). The filtrate was extracted with CH₂Cl₂ (3× 400 mL). The combined organic layers were washed with brine (2×500 mL), dried over anhydrous Na₂SO₄. After filtration, the filtrate was concentrated under reduced pressure. The residue was purified by trituration with PE/EA (6/1) (400 mL) to afford 5-bromo-4-fluoro-2,3-dihydroinden-1-one (65.3 g, crude) as a light yellow solid. ¹H NMR (400 MHz, DMSO-d₆) δ 7.81-7.71 (m, 1H), 7.42 (d, J=8.1 Hz, 1H), 3.20-3.07 (m, 2H), 2.80-2.64 (m, 2H).

N-(4-fluoro-1-oxo-2,3-dihydroinden-5-yl)acetamide

A mixture of 5-bromo-4-fluoro-2,3-dihydroinden-1-one (5.0 g, 21.8 mmol, 1.00 equiv), acetamide (2.6 g, 43.7 mmol, 2.00 equiv), Pd(OAc) 2 (250 mg, 1.09 mmol, 0.05 equiv), XantPhos (630 mg, 1.09 mmol, 0.05 equiv) and Cs₂CO₃ (14.2 g, 43.7 mmol, 2.00 equiv) in dioxane (50.0 mL) was stirred for 2 h at 100° C. under nitrogen atmosphere. The mixture was allowed to cool down to room temperature. The resulting mixture was diluted with CH₂Cl₂ (100 mL). The resulting mixture was filtered, the filter cake was washed with CH₂Cl₂ (3×30 mL). The filtrate was concentrated under reduced pressure. The residue was purified by silica gel column chromatography, eluted with PE/EA (5:1) to afford N-(4-fluoro-1-oxo-2,3-dihydroinden-5-yl) acetamide (1.07 g, 23.7%) as a yellow solid. LC-MS (ESI): m/z=205.85 [M−H]

5-amino-4-fluoro-2,3-dihydroinden-1-one

A mixture of N-(4-fluoro-1-oxo-2,3-dihydroinden-5-yl) acetamide (1.10 g, 5.16 mmol, 1.00 equiv) and K₂CO₃ (1.40 g, 10.3 mmol, 2.00 equiv) in MeOH (10.0 mL) was stirred for 2 h at 60° C. The mixture was allowed to cool down to room temperature. The resulting mixture was concentrated under reduced pressure. The resulting mixture was diluted with water (100 mL). The resulting mixture was extracted with EtOAc (3×100 mL). The combined organic layers were washed with brine (2× 100 mL), dried over anhydrous Na₂SO₄. After filtration, the filtrate was concentrated under reduced pressure. This resulted in 5-amino-4-fluoro-2,3-dihydroinden-1-one (780 mg, crude) as a yellow solid. LC-MS (ESI): m/z 166.20 [M+H]⁺

4-fluoro-5-nitro-2,3-dihydroinden-1-one

To a stirred solution of 5-amino-4-fluoro-2,3-dihydroinden-1-one (4.60 g, 27.9 mmol, 1.00 equiv) (crude) in DCM (60.0 mL) was added m-CPBA (15.0 g, 86.9 mmol, 3.12 equiv) in portions at 0° C. The resulting mixture was stirred for 1 h at room temperature. The reaction was quenched with sat. NaHCO₃ (aq.) at 0° C. The resulting mixture was extracted with CH₂Cl₂ (3×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford 4-fluoro-5-nitro-2,3-dihydroinden-1-one (1.1 g, 20.2%) as a yellow solid. ¹H NMR (400 MHz, DMSO-d₆) δ 8.21 (dd, J=8.2, 6.4 Hz, 1H), 7.71 (d, J=8.2 Hz, 1H), 3.32-3.21 (m, 2H), 2.92-2.81 (m, 2H).

4-fluoro-5-nitro-2,3-dihydro-1H-inden-1-ol

To a stirred solution of 4-fluoro-5-nitro-2,3-dihydroinden-1-one (1.10 g, 5.64 mmol, 1.00 equiv) in MeOH (12.0 mL) was added NaBH₄ (640 mg, 16.9 mmol, 3.00 equiv) in portions at 0° C. The resulting mixture was stirred for 1 h at room temperature. The reaction was quenched with sat. NH₄Cl (aq.) at 0° C. The resulting mixture was diluted with water (50 mL). The resulting mixture was extracted with CH₂Cl₂ (3×80 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (1:1) to afford 4-fluoro-5-nitro-2,3-dihydro-1H-inden-1-ol (910 mg, 81.9%) as a yellow solid.

### Synthesis of 6-fluoro-5-nitro-2,3-dihydro-1H-inden-1-ol (Int.5)

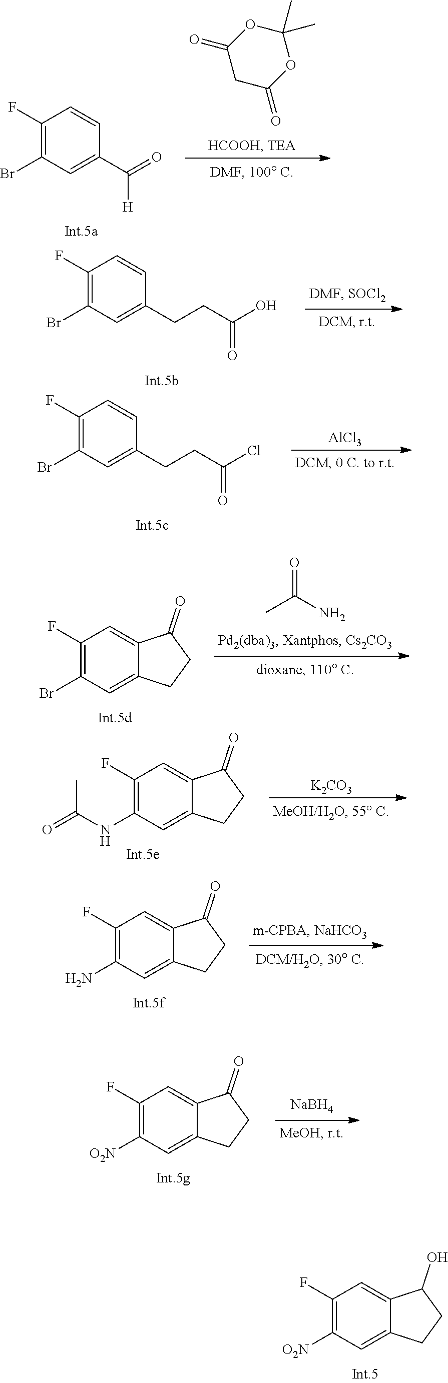

3-(3-bromo-4-fluorophenyl)propanoic acid

A mixture of TEA (32.5 g, 322 mmol, 1.3 eq.) and HCOOH (34.2 g, 744 mmol, 3.0 eq.) was stirred at room temperature for 15 min, then DMF (1.5 L) was added. To the solution were added 3-bromo-4-fluorobenzaldehyde (50 g, 248 mmol, 1.0 eq.) and 2,2-dimethyl-1,3-dioxane-4,6-dione (35.7 g, 248 mmol, 1.0 eq.). The reaction solution was stirred at 100° C. overnight. After completion, the reaction solution was poured into concentrated HCl/ice water (1 L) and the suspension was filtered to give 3-(3-bromo-2-fluorophenyl)propanoic acid (57 g, 231 mol, 93%) as a white solid, which was used in next step directly without further purification. LC-MS (ESI): m/z 247 [M+H]⁺.

3-(3-bromo-4-fluorophenyl)propanoyl chloride

To a solution of 3-(3-bromo-2-fluorophenyl) propanoic acid (57 g, 231 mmol, 1.0 eq.) in DCM (500 mL) was added SOCl₂ (50 mL, 693 mmol, 3.0 eq.) followed with DMF (169 mg, 2.31 mmol, 0.01 eq.). The solution was stirred at room temperature for 12 hrs. After completion, the reaction solution was concentrated under reduced pressure to give crude 3-(3-bromo-2-fluorophenyl) propanoyl chloride (58 g, quant) as a yellow oil, which was used in next step directly without further purification. LC-MS (ESI): m/z 265 [M+H]⁺.

5-bromo-6-fluoro-2,3-dihydro-1H-inden-1-one

To a solution of AlCl₃ (43.9 g, 330 mmol, 1.5 eq.) in DCM (500 mL) was added the solution of 3-(3-bromo-2-fluorophenyl) propanoyl chloride (58 g, 220 mmol, 1.0 eq.) in DCM (500 mL) at 0° C. The solution was stirred at 0° C. for 15 min, then warmed up to room temperature and stirred for 3 hrs. After completion, the solution was poured into ice water (500 mL) and the resulting suspension were filtered. The filtrate was extracted with DCM (500 mL×2). The organic layers were combined, dried over anhydrous Na₂SO₄, and concentrated under reduced pressure. The residue was purified by flash column chromatography on silica gel to afford 5-bromo-6-fluoro-2,3-dihydro-1H-inden-1-one (19.5 g, 85.5 mmol, 39%) as a white solid. LC-MS (ESI): m/z 229 [M+H]⁺.

N-(6-fluoro-1-oxo-2,3-dihydro-1H-inden-5-yl)acetamide

To a solution of 5-bromo-6-fluoro-2,3-dihydro-1H-inden-1-one (1.00 g, 4.39 mmol, 1.0 eq.) in dioxane (30 mL) were added acetamide (518 mg, 8.77 mmol, 2.0 eq.), CS₂CO₃ (2.86 g, 8.77 mmol, 2.0 eq.), Pd₂(dba)₃ (403 mg, 0.44 mmol, 0.1 eq.) and Xantphos (510 mg, 0.88 mmol, 0.2 eq.). The solution was stirred at 110° C. for 12 hrs under nitrogen atmosphere. After completion, the reaction solution was cooled to room temperature, then extracted with EtOAc (30 mL×3). The organic layers were combined, dried over anhydrous Na₂SO₄ and concentrated under reduced pressure. The residue was purified by flash column chromatography on silica gel to afford N-(6-fluoro-1-oxo-2,3-dihydro-1H-inden-5-yl)acetamide (850 mg, 4.10 mmol, 94%) as a yellow solid. LC-MS (ESI): m/z 208 [M+H]⁺.

5-amino-6-fluoro-2,3-dihydro-1H-inden-1-one

To a solution of N-(6-fluoro-1-oxo-2,3-dihydro-1H-inden-5-yl) acetamide (6.00 g, 29.0 mmol, 1.0 eq.) in MeOH (60 mL) was added K₂CO₃ (60 mL, sat., aq.). The reaction solution was stirred at 55° C. for 16 hrs. After completion, the reaction solution was diluted with H₂O (60 mL) and extracted with EtOAc (60 mL×3). The organic layers were combined, dried over anhydrous Na₂SO₄ and concentrated under reduced pressure. The residue was purified by flash column chromatography on silica gel to afford 5-amino-6-fluoro-2,3-dihydro-1H-inden-1-one (4.3 g, 25.9 mmol, 89%) as a yellow solid. LC-MS (ESI): m/z 166 [M+H]⁺.

6-fluoro-5-nitro-2,3-dihydro-1H-inden-1-one

To a solution of 5-amino-6-fluoro-2,3-dihydro-1H-inden-1-one (8.9 g, 53.9 mmol, 1.0 eq.) and NaHCO₃ (23.5 g, 270 mmol, 5.0 eq.) in a mixture of DCM (450 mL) and H₂O (20 mL) was added m-CPBA (55.0 g, 85 wt %, 270 mmol, 5.0 eq.) in portions. After addition, the reaction mixture was stirred at 30° C. for 2 hrs. After completion, the reaction mixture was cooled down in an ice bath, then neutralized carefully with NaHCO₃ (aq.) until the pH was adjusted to pH=8. The resulting mixture was extracted with EtOAc (150 mL×3). The combined organic layers were washed with brine (10 mL×2), dried over anhydrous Na₂SO₄ and concentrated under reduced pressure. The residue was purified by flash column chromatography on silica gel to afford 6-fluoro-5-nitro-2,3-dihydro-1H-inden-1-one (4.2 g, 21.5 mmol, 40%) as a yellow oil. LC-MS (ESI): m/z 196 [M+H]⁺.

6-fluoro-5-nitro-2,3-dihydro-1H-inden-1-ol

To a solution of 6-fluoro-5-nitro-2,3-dihydro-1H-inden-1-one (45 mg, 0.23 mmol, 1.0 eq.) in MeOH (5 mL) was added NaBH₄ (13 mg, 0.35 mmol, 1.5 eq.). The reaction solution was stirred at room temperature for 1 hr. After completion, the reaction mixture was concentrated under reduced pressure. The residue was poured into water (10 mL) and extracted with EtOAc (10 mL×3). The organic layers were combined and washed with brine (50 mL), dried over anhydrous Na₂SO₄ and concentrated under reduced pressure to afford 6-fluoro-5-nitro-2,3-dihydro-1H-inden-1-ole (35 mg, 0.18 mmol, 78%) as an oil. LC-MS (ESI): m/z 198 [M+H]⁺.

### Synthesis of 4-fluoro-5-nitro-2,3-dihydro-1H-inden-1-yl di(aziridin-1-yl)phosphinate (Int.6)

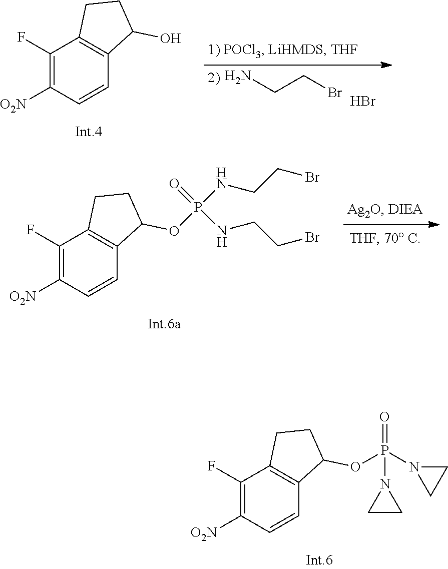

(2-bromoethyl)({[(2-bromoethyl)amino][(6-fluoro-5-nitro-2,3-dihydro-1H-inden-1-yl)oxy]phosphoryl})amine

To a solution of 4-fluoro-5-nitro-2,3-dihydro-1H-inden-1-ol (400 mg, 2.03 mmol, 1.0 eq.) in THF (5 mL) was added LiHMDS (2.23 mL, 1 M in THF, 2.23 mmol, 1.1 eq.) dropwise at −65° C. under N₂ and the resulting solution was stirred at −65° C. for 20 min under N₂. POCl₃ (621 mg, 4.06 mmol, 2.0 eq.) was added and the resulting mixture was stirred at −65° C. for 20 min. 2-Bromoethylamine hydrobromide (2497 mg, 12.2 mmol, 6.0 eq.) and TEA (2.46 g, 24.4 mmol, 12.0 eq.) were added into the above mixture. The resulting mixture was warmed up to room temperature and stirred for 1 hr. After completion, the reaction mixture was quenched with NH₄Cl solution (50 mL, sat., aq.) and extracted with EtOAc (50 mL×2). The organic layers were combined and washed with brine (20 mL), dried over anhydrous Na₂SO₄ and concentrated under reduced pressure. The residue was purified by flash column chromatography on silica gelt to afford 4-fluoro-5-nitro-2,3-dihydro-1H-indenyl P,P-bis[(2-bromoethyl)amino]phosphinate (200 mg, 0.41 mmol, 20%) as a yellow oil. LC-MS (ESI): m/z 488 [M+H]⁺.

4-fluoro-5-nitro-2,3-dihydro-1H-inden-1-yl di(aziridin-1-yl)phosphinate

To a solution of 4-fluoro-5-nitro-2,3-dihydro-1H-indenyl P,P-bis[(2-bromoethyl)amino]phosphinate (200 mg, 0.41 mmol, 1.0 eq.) in THF (5 mL) were added Ag₂O (951 mg, 4.10 mmol, 10.0 eq.) and DIEA (529 mg, 4.10 mmol, 10.0 eq.). The resulting solution was stirred at 70° C. for 18 hrs under N₂. After completion, the reaction mixture was filtered, and the filtrate was concentrated under reduced pressure. The residue was purified by Biotage® C18 column chromatography to afford 4-fluoro-5-nitro-2,3-dihydro-1H-inden-1-yl di(aziridin-1-yl)phosphinate (84 mg, 0.26 mmol, 63%) as a solid. LC-MS (ESI): m/z 328 [M+H]⁺.

### Illustration 1. Synthesis of (S)-1-(3-((4-methoxy-2-oxopyridin-1 (2H)-yl)methyl)-4-nitrophenyl)ethyl di(aziridin-1-yl)phosphinate (2) & (S)-1-(3-(((4-methoxypyridin-2-yl)oxy)methyl)-4-nitrophenyl)ethyl di(aziridin-1-yl)phosphinate (3) (General Procedure 1)

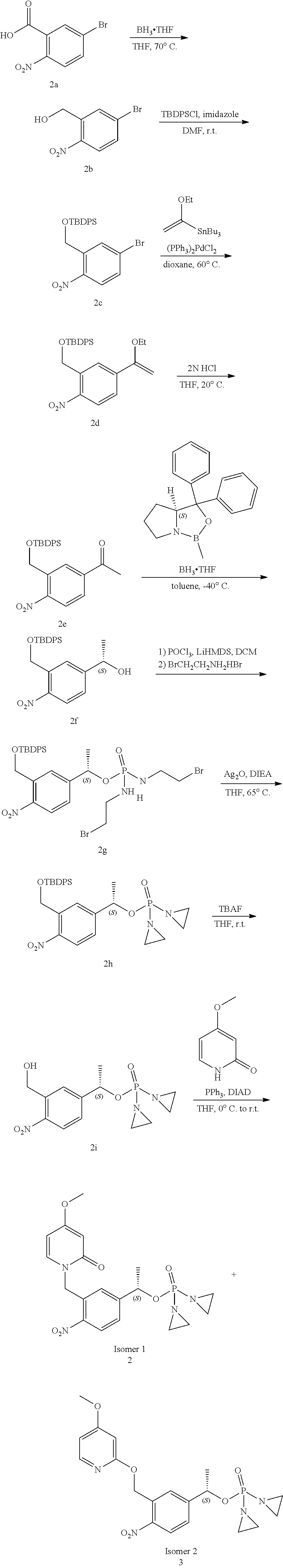

(5-bromo-2-nitrophenyl)methanol

To a solution of 5-bromo-2-nitrobenzoic acid (5.0 g, 20.3 mmol, 1.0 eq.) in THF (20 mL) was added BH₃·THF (41 mL, 1 N, 41 mmol, 2.0 eq.) at room temperature under nitrogen. The solution was heated at 70° C. for 3 hours. After completion, the reaction mixture was cooled to 0° C., and then quenched with 2 N HCl solution (20 mL, aq.). The mixture was extracted with EtOAc (50 mL×2). The organic layers were combined and washed with brine (50 mL), dried over anhydrous Na₂SO₄ and concentrated under reduced pressure to afford (5-bromo-2-nitrophenyl) methanol (4.0 g, 17.2 mmol, 85%) as a yellow solid. ¹H NMR (400 MHz, CDCl₃) δ 8.01-7.98 (m, 2H), 7.60 (dd, J=8.7, 2.2 Hz, 1H), 5.02 (s, 2H).

((5-bromo-2-nitrobenzyl)oxy)(tert-butyl)diphenylsilane

To a solution of (5-bromo-2-nitrophenyl) methanol (4.0 g, 4.0 g, 17.2 mmol, 1.0 eq.) in DMF (40 mL) were added Imidazole (1.41 g, 20.7 mmol, 1.2 eq.) and TBDPSCl (4.46 g, 17.2 mmol, 1.0 eq.) at room temperature. After addition, the reaction mixture was stirred at room temperature for 16 hrs. After completion, the reaction mixture was diluted with water (100 mL) and extracted with EtOAc (100 mL×2). The organic layers were combined and washed with brine (50 mL), dried over anhydrous Na₂SO₄, and concentrated under reduced pressure. The residue was purified by flash column chromatography on silica gel to afford ((5-bromo-2-nitrobenzyl)oxy) (tert-butyl)diphenylsilane (7.2 g, 15.3 mmol, 89%) as a yellow oil. ¹H NMR (400 MHz, CDCl₃) δ 8.31-8.18 (m, 1H), 7.96 (d, J=8.7 Hz, 1H), 7.69-7.63 (m, 4H), 7.56 (dd, J=8.7, 2.2 Hz, 1H), 7.45-7.36 (m, 6H), 5.12 (s, 2H), 1.14 (s, 9H).

tert-butyl((5-(1-ethoxyvinyl)-2-nitrobenzyl)oxy)diphenylsilane

To a solution of ((5-bromo-2-nitrobenzyl)oxy) (tert-butyl)diphenylsilane (7.2 g, 15.3 mmol, 1.0 eq.) in dioxane (110 mL) were added tributyl(1-ethoxyvinyl) stannane (5.2 mL, 15.3 mmol, 1.0 eq.) and (PPh₃)₂PdCl₂ (0.32 g, 0.46 mmol, 0.03 eq.). The mixture was evaporated and backfilled with nitrogen for three times. The mixture was stirred at 60° C. for 16 hrs. After completion, the reaction mixture was cooled to room temperature, quenched with KF solution (100 mL) and extracted with EtOAc (20 mL×3). The combined organic layers were dried over anhydrous Na₂SO₄, filtered and concentrated under reduced pressure. The residue was purified by flash column chromatography on silica gel to give tert-butyl ((5-(1-ethoxyvinyl)-2-nitrobenzyl)oxy)diphenylsilane (6.1 g, 13.2 mmol, 86%) as a yellow solid. ¹H NMR (400 MHz, CDCl₃) δ 8.51-8.39 (m, 1H), 8.07 (d, J=8.6 Hz, 1H), 7.75-7.60 (m, 5H), 7.49-7.33 (m, 6H), 5.16 (s, 2H), 4.85 (d, J=3.0 Hz, 1H), 4.40 (d, J=3.0 Hz, 1H), 3.96 (q, J=7.0 Hz, 2H), 1.45 (t, J=7.0 Hz, 3H), 1.15 (s, 9H).

1-(3-(((tert-butyldiphenylsilyl)oxy)methyl)-4-nitrophenyl)ethan-1-one

To a solution of tert-butyl ((5-(1-ethoxyvinyl)-2-nitrobenzyl)oxy)diphenylsilane (6.1 g, 13.2 mmol, 1.0 eq.) in THF (40 mL) was added HCl aqueous solution (40 mL, 2 N) and the mixture was stirred at 20° C. for 2 hrs under nitrogen atmosphere. After completion, the mixture was poured into water (20 mL) and extracted with EtOAc (10 mL×3). The organic layers were combined, dried over anhydrous Na₂SO₄ and concentrated under reduced pressure to give 1-(3-(((tert-butyldiphenylsilyl)oxy)methyl)-4-nitrophenyl)ethan-1-one (15.6 g, quant) as a yellow oil, which was used in next step directly without further purification. ¹H NMR (400 MHz, CDCl₃) δ 8.72-8.53 (m, 1H), 8.11 (d, J=8.5 Hz, 1H), 8.00 (d, J=1.9 Hz, 1H), 7.75-7.61 (m, 4H), 7.51-7.31 (m, 6H), 5.17 (s, 2H), 2.66 (s, 3H), 1.15 (s, 9H).

(S)-1-(3-(((tert-butyldiphenylsilyl)oxy)methyl)-4-nitrophenyl)ethan-1-ol

To a solution of (S)-1-methyl-3,3-diphenyltetrahydro-1H,3H-pyrrolo[1,2-c][1,3,2]oxazaborole (1.50 g, 5.17 mmol, 0.3 eq.) in toluene (6 mL) was added BH₃·THF (15 mL, 1 N, 15.0 mmol, 1.1 eq.) at 0° C. under nitrogen. The solution was stirred at 0° C. for 30 min, then cooled to −40° C. A solution of 1-(3-(((tert-butyldiphenylsilyl)oxy)methyl)-4-nitrophenyl)ethan-1-one (5.6 g, 12.9 mmol, 1.0 eq.) in THF (60 mL) was added slowly to the above mixture at −40° C. After addition, the reaction mixture was stirred at −40° C. for 2 hrs. MeOH (20 mL) was added into the reaction mixture at −40° C., and the solution was stirred for 30 min. The mixture was concentrated under reduced pressure. The residue was purified by flash column chromatography on silica gel to give (S)-1-(3-(((tert-butyldiphenylsilyl)oxy)methyl)-4-nitrophenyl)ethan-1-ol (4.3 g, 9.87 mmol, 76%) as a yellow solid. ¹H NMR (400 MHz, CDCl₃) δ 8.07 (d, J=8.4 Hz, 2H), 7.73-7.59 (m, 4H), 7.48-7.32 (m, 7H), 5.17 (s, 2H), 5.08-4.91 (m, 1H), 1.53 (d, J=6.5 Hz, 3H), 1.14 (s, 9H).

(S)-1-(3-(((tert-butyldiphenylsilyl)oxy)methyl)-4-nitrophenyl)ethyl di((2-bromoethyl)amino)phosphinate

To a solution of (S)-1-(3-(((tert-butyldiphenylsilyl)oxy)methyl)-4-nitrophenyl)ethan-1-ol (3.5 g, 8.04 mmol, 1.0 eq.) in THF (20 mL) was added LiHMDS (12 mL, 12.0 mmol, 1N in THE solution, 1.5 eq.) at −40° C. under nitrogen atmosphere and the resulting mixture was stirred for 20 min. To the mixture was added POCl₃ (1.5 mL, 16.1 mmol, 2.0 eq.) at −40° C. and the reaction solution was stirred for another 20 min. Then, 2-bromoethan-1-amine hydrobromide (1.88 g, 9.18 mmol, 1.1 eq.) and DIEA (10.6 mL, 64.3 mmol, 8.0 eq.) were added. The resulting mixture was warmed up to room temperature and stirred for 1 hr. After completion, the reaction mixture was quenched with sat. NH₄Cl solution (40 mL) and extracted with DCM (40 mL×3). The organic layers were combined and washed with water (10 mL), dried over anhydrous Na₂SO₄ and concentrated under reduced pressure. The residue was purified by Biotage® C18 column chromatography to give

(S)-1-(3-(((tert-butyldiphenylsilyl)oxy)methyl)-4-nitrophenyl)ethyl di((2-bromoethyl)amino)phosphinate (2.8 g, 3.85 mmol, 48%) as a yellow solid. ¹H NMR (400 MHz, CDCl₃) δ 8.14-8.04 (m, 2H), 7.68-7.63 (m, 4H), 7.47-7.34 (m, 7H), 5.63-5.60 (m, 1H), 5.18 (s, 2H), 3.52-3.48 (m, 2H), 3.41-3.34 (m, 2H), 3.27-3.05 (m, 4H), 1.64 (d, J=6.5 Hz, 3H), 1.15 (s, 9H).
(S)-1-(3-(((tert-butyldiphenylsilyl)oxy)methyl)-4-nitrophenyl)ethyl di(aziridin-1-yl)phosphinate

To a solution of (S)-1-(3-(((tert-butyldiphenylsilyl)oxy)methyl)-4-nitrophenyl)ethyl di((2-bromoethyl)amino)phosphinate (2.8 g, 3.85 mmol, 1.0 eq.) and DIEA (3.2 mL, 19.2 mmol, 5.0 eq.) in THF (20 mL) was added Ag₂O (4.46 g, 19.2 mmol, 5.0 eq.). The mixture was stirred at 65° C. for 16 hrs under N₂. After completion, the reaction mixture was cooled to room temperature and filtered. The filtrate was concentrated under reduced pressure. The residue was purified by Biotage® C18 column chromatography to give (S)-1-(3-(((tert-butyldiphenylsilyl)oxy)methyl)-4-nitrophenyl)ethyl di(aziridin-1-yl)phosphinate (1.1 g, 1.95 mmol, 51%) as a yellow oil. ¹H NMR (400 MHz, CDCl₃) δ 8.11-8.06 (m, 2H), 7.69-7.64 (m, 4H), 7.47-7.36 (m, 7H), 5.80-5.65 (m, 1H), 5.16 (s, 2H), 2.27-1.98 (m, 8H), 1.65 (d, J=6.5 Hz, 3H), 1.14 (s, 9H). ³¹P NMR (162 MHz, CDCl₃) δ 30.01 (s). LC-MS (ESI): m/z 588.3 [M+Na]⁺.

(S)-1-(3-(hydroxymethyl)-4-nitrophenyl)ethyl di(aziridin-1-yl)phosphinate

To a solution of (S)-1-(3-(((tert-butyldiphenylsilyl)oxy)methyl)-4-nitrophenyl)ethyl di(aziridin-1-yl)phosphinate (1.1 g, 1.95 mmol, 1.0 eq.) in THF (3 mL) was added TBAF (2.9 mL, 2.91 mmol, 1.5 eq., 1M) and the resulting mixture was stirred at room temperature for 30 min under N₂. After completion, the reaction mixture was diluted with water (20 mL) and extracted with EtOAc (30 mL×3). The organic layers were combined and washed with water (20 mL), dried over anhydrous Na₂SO₄ and concentrated under reduced pressure. The residue was purified by Biotage® C18 column chromatography to give (S)-1-(3-(hydroxymethyl)-4-nitrophenyl)ethyl di(aziridin-1-yl)phosphinate (340 mg, 1.04 mmol, 53%) as a yellow oil. ¹H NMR (400 MHz, CDCl₃) δ 8.12 (d, J=8.4 Hz, 1H), 7.79 (s, 1H), 7.50-7.48 (m, 1H), 5.74-5.67 (m, 1H), 5.00 (s, 2H), 2.25-2.01 (m, 8H), 1.64 (d, J=6.6 Hz, 3H). ³¹P NMR (162 MHz, CDCl₃) δ 30.27 (s). LC-MS (ESI): m/z 328.1 [M+H]⁺.

(S)-1-(3-((4-methoxy-2-oxopyridin-1 (2H)-yl)methyl)-4-nitrophenyl)ethyl di(aziridin-1-yl)phosphinate & (S)-1-(3-(((4-methoxypyridin-2-yl)oxy)methyl)-4-nitrophenyl)ethyl di(aziridin-1-yl)phosphinate

To a solution of 4-methoxypyridin-2 (1H)-one (50 mg, 0.40 mmol, 1.5 eq.), PPh₃ (210 mg, 0.80 mmol, 3.0 eq.) and(S)-1-(3-(hydroxymethyl)-4-nitrophenyl)ethyl di(aziridin-1-yl)phosphinate (87.2 mg, 0.27 mmol, 1.0 eq.) in THF (12 mL) was added DIAD (0.13 mL, 0.67 mmol, 2.5 eq.) at 0° C. The resulting mixture was warmed up to room temperature and stirred for 1 hr under N₂. After completion, the reaction solution was concentrated under reduced pressure. The residue was purified by RP-prep HPLC to give (S)-1-(3-((4-methoxy-2-oxopyridin-1(2H)-yl)methyl)-4-nitrophenyl)ethyl di(aziridin-1-yl)phosphinate (35 mg, 0.08 mmol, 30%) as a white solid and (S)-1-(3-(((4-methoxypyridin-2-yl)oxy)methyl)-4-nitrophenyl)ethyl di(aziridin-1-yl)phosphinate (1.2 mg) as a yellow oil.

Isomer 1(2), 35 mg, 0.08 mmol, 30%, ¹H NMR (400 MHz, CDCl₃) δ 8.12 (d, J=8.5 Hz, 1H), 7.49 (dd, J=8.5, 1.7 Hz, 1H), 7.18 (d, J=7.6 Hz, 1H), 7.08 (s, 1H), 6.01-5.94 (m, 2H), 5.65-5.55 (m, 1H), 5.46 (d, J=1.9 Hz, 2H), 2.21-1.98 (m, 8H), 1.56 (d, J=6.5 Hz, 3H). ³¹P NMR (162 MHz, CDCl₃) δ 29.89 (s). LC-MS (ESI): m/z 435.2 [M+H]⁺.

Isomer 2(3), 1.2 mg, ¹H NMR (400 MHz, CDCl₃) δ 8.19-8.08 (m, 1H), 7.97-7.87 (m, 1H), 7.76-7.67 (m, 1H), 7.54-7.41 (m, 1H), 6.57-6.44 (m, 1H), 6.41-6.27 (m, 1H), 5.87-5.73 (m, 2H), 5.72-5.47 (m, 1H), 3.85 (s, 3H), 2.23-1.90 (m, 8H), 1.60 (d, J=6.4 Hz, 3H). ³¹P NMR (162 MHz, CDCl₃) δ 30.24 (s). LC-MS (ESI): m/z 435.2 [M+H]⁺.

### Illustration 2. Synthesis of 4-((3-(3-(dimethylcarbamoyl)phenoxy)-4-nitrobenzyl)oxy)-2,3,5,6-tetrafluorobenzyl bis((S)-2-methylaziridin-1-yl)phosphinate (12) (General Procedure 5)

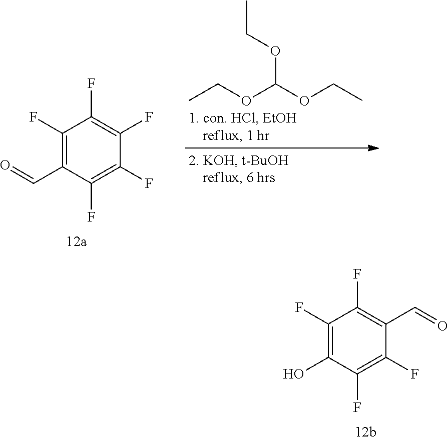

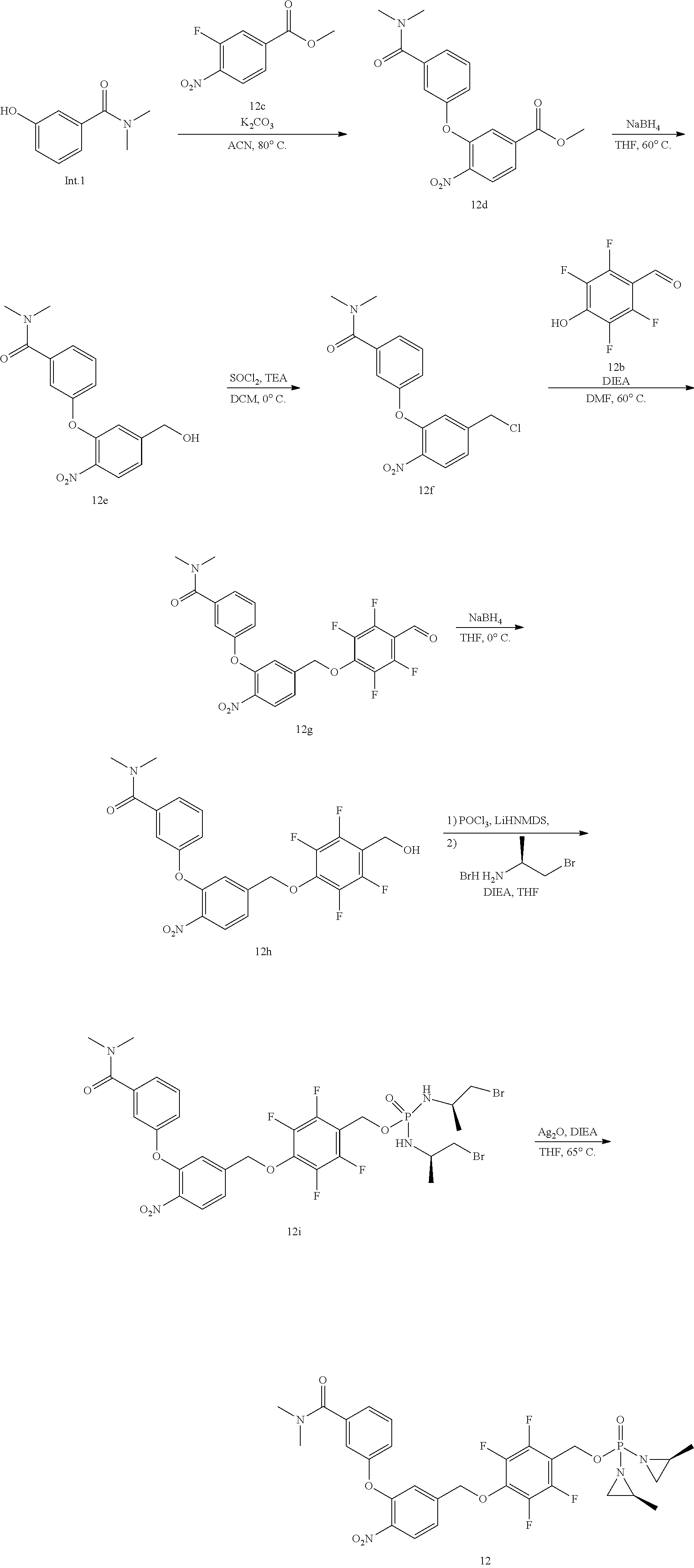

2,3,5,6-tetrafluoro-4-hydroxybenzaldehyde

A solution of 2,3,4,5,6-pentafluorobenzaldehyde (10.0 g, 51.0 mmol, 1.0 eq.), (diethoxymethoxy) ethane (11.0 mL, 66.3 mmol, 1.3 eq.) and 12 N HCl (0.15 mL) in EtOH (30 mL) was heated under reflux for overnight. The reaction mixture was concentrated under reduced pressure to give 1-(diethoxymethyl)-2,3,4,5,6-pentafluorobenzene (11.2 g, 41.4 mmol, 75%) as a colorless oil. The residue was dissolved in t-BuOH (150 mL) and KOH (9.3 g, 165.8 mmol, 4.0 eq.) was added at room temperature, then the reaction mixture was stirred at 85° C. for 4 hrs. After completion, the reaction mixture was cooled down in an ice bath, then neutralized carefully with HCl (12 N) until the pH was adjusted to pH=3. The resulting mixture was extracted with EtOAc (100 mL×2), and the combined organic layers were washed with brine, dried over with anhydrous Na₂SO₄, then concentrated under reduced pressure. The residue was purified by flash column chromatography on silica gel to afford 2,3,5,6-tetrafluoro-4-hydroxybenzaldehyde (5.1 g, 26.3 mmol, 57%) as a white solid. LCMS (ESI): m/z 193 [M−H]⁻.

methyl 3-(3-(dimethylcarbamoyl)phenoxy)-4-nitrobenzoate

To a solution of 3-hydroxy-N,N-dimethylbenzamide (580 mg, 3.51 mmol, 1.0 eq.) and methyl 3-fluoro-4-nitrobenzoate (699 mg, 3.51 mmol, 1.0 eq.) in DMF (5 mL) was added K₂CO₃ (534 mg, 3.86 mmol, 1.1 eq.) under nitrogen and the reaction mixture was stirred at 80° C. for 18 hrs. After completion, the reaction mixture was diluted with H₂O (25 mL), extracted with EtOAc (25 mL×3). The organic layers were combined and washed with brine (20 mL), dried over anhydrous Na₂SO₄, and concentrated under reduced pressure. The residue was purified by Biotage® C18 column chromatography to afford methyl 3-(3-(dimethylcarbamoyl)phenoxy)-4-nitrobenzoate (1.1 g, 3.19 mmol, 91%) as a white solid. LCMS (ESI): m/z 345 [M+H]⁺.

3-(5-(hydroxymethyl)-2-nitrophenoxy)-N,N-dimethylbenzamide

To a solution of methyl 3-(3-(dimethylcarbamoyl)phenoxy)-4-nitrobenzoate (1.1 g, 3.2 mmol, 1.0 eq.) in THF (20 mL) was added NaBH₄ (969 mg, 25.5 mmol, 8.0 eq.) in portions under nitrogen and then the reaction mixture was stirred at 60° C. for 18 hrs. After completion, the reaction mixture was quenched by adding sat. NH₄Cl aqueous solution (20 mL), then extracted with EtOAc (40 mL×3). The organic layers were combined and washed with brine (20 mL), dried over anhydrous Na₂SO₄, and concentrated under reduced pressure. The residue was purified by Biotage® C18 column chromatography to afford 3-(5-(hydroxymethyl)-2-nitrophenoxy)-N,N-dimethylbenzamide (1.0 g, 3.2 mmol, 98%) as a white solid. LC-MS (ESI) m/z 317 [M+H]⁺. ¹H NMR (400 MHz, CDCl₃) δ 7.95 (d, J=8.4 Hz, 1H), 7.41 (t, J=7.9 Hz, 1H), 7.21-7.15 (m, 2H), 7.13-7.10 (m, 1H), 7.08-7.06 (m, 1H), 7.03-7.02 (m, 1H), 4.64 (s, 2H), 3.07 (s, 3H), 2.97 (s, 3H).

3-(5-(chloromethyl)-2-nitrophenoxy)-N,N-dimethylbenzamide

To a solution of 3-(5-(hydroxymethyl)-2-nitrophenoxy)-N,N-dimethylbenzamide (900 mg, 2.8 mmol, 1.0 eq.) in DCM (15 mL) was added SOCl₂ (0.4 mL, 5.7 mmol, 2.0 eq.) dropwise at 0° C. under nitrogen. After addition, the mixture was stirred at room temperature for 1 hr. Then TEA (0.4 mL, 2.8 mmol, 1.0 eq.) was added and the reaction mixture was stirred at room temperature for additional 30 min. After completion, the reaction mixture was cooled down in an ice bath, then quenched with NaHCO₃ solution (aq.). The resulting mixture was extracted with DCM (30 mL×2), and the combined organic layers were washed with water (10 mL×2), dried over anhydrous Na₂SO₄, then concentrated under reduced pressure to give crude 3-(5-(chloromethyl)-2-nitrophenoxy)-N,N-dimethylbenzamide (820 mg, quant) as a yellow solid, which was used in next step directly without further purification. LCMS (ESI): m/z 335 [M+H]⁺. ¹H NMR (400 MHz, DMSO-d₆) δ 7.97 (d, J=8.4 Hz, 1H), 7.44 (t, J=7.9 Hz, 1H), 7.27 (d, J=1.8 Hz, 1H), 7.25-7.22 (m, 1H), 7.12-7.09 (m, 1H), 7.08-7.06 (m, 2H), 4.51 (s, 2H), 3.09 (s, 3H), 2.97 (s, 3H).

3-(5-(chloromethyl)-2-nitrophenoxy)-N,N-dimethylbenzamide

A solution of 3-(5-(chloromethyl)-2-nitrophenoxy)-N,N-dimethylbenzamide (820 mg, 2.45 mmol, 1.0 eq.), 2,3,5,6-tetrafluoro-4-hydroxybenzaldehyde (1.4 g, 7.3 mmol, 3.0 eq.) and DIEA (1.2 mL, 7.3 mmol, 3.0 eq.) in DMF (15 mL) was stirred at 60° C. for 16 hrs. After completion, the reaction mixture was concentrated under reduced pressure. The residue was purified by Biotage® C18 column chromatography to give 3-(5-(chloromethyl)-2-nitrophenoxy)-N,N-dimethylbenzamide (150 mg, 0.29 mmol, 12%) as a yellow solid. ¹H NMR (400 MHz, CDCl₃) δ 10.22 (s, 1H), 8.01 (d, J=8.4 Hz, 1H), 7.45-7.40 (m, 1H), 7.30 (dd, J=8.4, 1.7 Hz, 1H), 7.26-7.24 (m, 1H), 7.10 (m, 3H), 5.37 (s, 2H), 3.10 (s, 3H), 2.98 (s, 3H).

N,N-dimethyl-3-(2-nitro-5-((2,3,5,6-tetrafluoro-4-(hydroxymethyl)phenoxy)methyl)phenoxy)benzamide

To a solution of 3-(5-(chloromethyl)-2-nitrophenoxy)-N,N-dimethylbenzamide (150 mg, 0.30 mmol, 1.0 eq.) in THF (5 mL) was added NaBH₄ (34 mg, 0.61 mmol, 2.0 eq.) in portions at 0° C. and the reaction mixture was stirred at 0° C. for 1 hr. After completion, the reaction mixture was quenched with adding water (10 mL), extracted with DCM (20 mL×3). The combined organic phases were dried over anhydrous Na₂SO₄, filtered and concentrated under reduced pressure. The residue was purified by flash column chromatography on silica gel to give N,N-dimethyl-3-(2-nitro-5-((2,3,5,6-tetrafluoro-4-(hydroxymethyl)phenoxy)methyl)phenoxy)benzamide (120 mg, 0.24 mmol, 80%) as a white solid. LCMS (ESI): m/z 495 [M+H]⁺.

4-((3-(3-(dimethylcarbamoyl)phenoxy)-4-nitrobenzyl)oxy)-2,3,5,6-tetrafluorobenzyl di((R)-1-bromopropan-2-yl amino)phosphinate

To a solution of N,N-dimethyl-3-(2-nitro-5-((2,3,5,6-tetrafluoro-4-(hydroxymethyl)phenoxy)methyl)phenoxy)benzamide (210 mg, 0.43 mmol, 1.0 eq.) in THF (10 mL) was added LiHMDS (0.64 mL, 0.64 mmol, 1.5 eq.) at −78° C. under nitrogen. After addition, the mixture was stirred for 20 min at this temperature. Then, POCl₃ (130.26 mg, 0.85 mmol, 2.0 eq.) was added and the mixture was stirred at −78° C. for 20 min, (S)-1-bromopropan-2-amine hydrobromide (557.93 mg, 2.55 mmol, 5.9 eq.) and DIEA (0.562 mL, 3.40 mmol, 5.9 eq.) were added into the mixture. After addition, the reaction mixture was warmed to room temperature and stirred for 1 hr. After completion, the reaction mixture was quenched with sat. NH₄Cl solution (20 mL), extracted with DCM (80 mL). The organic phase was washed with water (10 mL), dried over anhydrous Na₂SO₄, filtered and concentrated under reduced pressure. The residue was purified by Biotage® C18 column chromatography to give 4-((3-(3-(dimethylcarbamoyl)phenoxy)-4-nitrobenzyl)oxy)-2,3,5,6-tetrafluorobenzyl di((R)-1-bromopropan-2-yl amino)phosphinate (45 mg, 55 μmol, 13%) as a yellow oil. LCMS (ESI): m/z 813.0 [M+H]⁺.

4-((3-(3-(dimethylcarbamoyl)phenoxy)-4-nitrobenzyl)oxy)-2,3,5,6-tetrafluorobenzyl bis((S)-2-methylaziridin-1-yl)phosphinate

To a solution of 4-((3-(3-(dimethylcarbamoyl)phenoxy)-4-nitrobenzyl)oxy)-2,3,5,6-tetrafluorobenzyl di((R)-1-bromopropan-2-yl amino)phosphinate (45 mg, 55 μmol, 1.0 eq.) and DIEA (35.71 mg, 0.28 mmol, 5.0 eq.) in THF (5 mL) was added Ag₂O (64.03 mg, 0.28 mmol, 5.0 eq.) and the reaction mixture was stirred at 65° C. for 16 hrs. After completion, the reaction mixture was cooled to room temperature and filtered. The filtrate was concentrated under reduced pressure to give a residue. The residue was purified by Biotage® C18 column chromatography to give 4-((3-(3-(dimethylcarbamoyl)phenoxy)-4-nitrobenzyl)oxy)-2,3,5,6-tetrafluorobenzyl bis((S)-2-methylaziridin-1-yl)phosphinate (22.2 mg, 34 μmol, 62%) as a colorless oil. ¹H NMR (400 MHz, CDCl₃) δ 8.01 (d, J=8.4 Hz, 1H), 7.48-7.44 (m, 1H), 7.34-7.31 (m, 1H), 7.28-7.25 (m, 1H), 7.15-7.09 (m, 3H), 5.25 (s, 2H), 5.21 (d, J=5.8 Hz, 2H), 3.11 (s, 3H), 3.00 (s, 3H), 2.64-2.52 (m, 2H), 2.43-2.33 (m, 2H), 1.96-1.85 (m, 2H), 1.30-1.28 (m, 6H). ³¹P NMR (162 MHz, CDCl₃) δ 29.22 (s) ¹⁹F NMR (376 MHz, CDCl₃) δ −143.13-143.20 (m), −156.15-−156.24 (m). LCMS (ESI): m/z 653.2 [M+H]⁺.

### Illustration 3. Synthesis of 5-nitro-4-phenoxy-2,3-dihydro-1H-inden-1-yl di(aziridin-1-yl)phosphinate (16) (General Procedure 8, 6,5 core 2-method A)

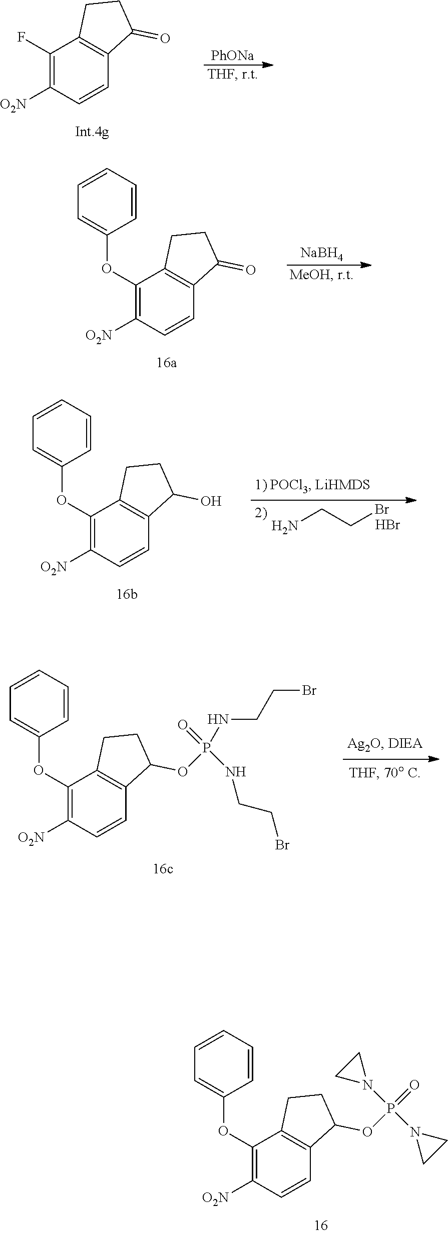

5-nitro-4-phenoxy-2,3-dihydro-1H-inden-1-one

To a solution of 4-fluoro-5-nitro-2,3-dihydro-1H-inden-1-one (30 mg, 0.15 mmol, 1.0 eq.) in THF (3 mL) was added phenoxysodium (19.6 mg, 0.17 mmol, 1.1 eq.) at 0° C. The solution was warmed up at room temperature and stirred for 3 hrs. After completion, the solution was concentrated under reduced pressure. The residue was purified by flash column chromatography on silica gel to afford 5-nitro-4-phenoxy-2,3-dihydro-1H-inden-1-one (28 mg, 0.10 mmol, 68%) as a yellow solid. ¹H NMR (400 MHz, CDCl₃) δ 7.91 (d, J=8.2 Hz, 1H), 7.71 (d, J=8.2 Hz, 1H), 7.38-7.30 (m, 2H), 7.16-7.09 (m, 1H), 6.97-6.89 (m, 2H), 2.85 (dd, J=7.2, 4.6 Hz, 2H), 2.75-2.67 (m, 2H).

5-nitro-4-phenoxy-2,3-dihydro-1H-inden-1-ol

To a solution of 5-nitro-4-phenoxy-2,3-dihydro-1H-inden-1-one (100 mg, 0.37 mmol, 1.0 eq.) in MeOH (5 mL) was added NaBH₄ (33.8 mg, 1.86 mmol, 5.0 eq.). The reaction solution was stirred at room temperature for 1 hr. After completion, the solution was concentrated under reduced pressure. The residue was purified by flash column chromatography on silica gel to afford 5-nitro-4-phenoxy-2,3-dihydro-1H-inden-1-ol (88 mg, 0.32 mmol, 87%) as a green oil.

5-nitro-4-phenoxy-2,3-dihydro-1H-inden-1-yl di((2-bromoethyl)amino)phosphinate

To a solution of 5-nitro-4-phenoxy-2,3-dihydro-1H-inden-1-ol (40 mg, 0.15 mmol, 1.0 eq.) in THF (10 mL) was added LiHMDS (0.15 mL, 1N in THE solution, 0.15 mmol, 1.0 eq.) at −40° C. under nitrogen atmosphere and the resulting mixture was stirred for 20 min. To the mixture was added POCl₃ (0.03 mL, 0.30 mmol, 2.0 eq.) at −40° C. and the reaction solution was stirred for another 20 min. Then, 2-bromoethan-1-amine hydrobromide (109.5 mg, 0.89 mmol, 6.0 eq.) and DIEA (0.194 mL, 1.18 mmol, 8.0 eq.) were added into the mixture and stirred at −40° C. for 10 min. The reaction mixture was warmed up at room temperature and stirred for 1 hr. After completion, the reaction mixture was concentrated under reduced pressure. The residue was purified by Biotage® C18 column chromatography to give 5-nitro-4-phenoxy-2,3-dihydro-1H-inden-1-yl di((2-bromoethyl)amino)phosphinate (30 mg, 0.055 mmol, 37%) as a yellow oil. LCMS (ESI): m/z 584 [M+Na]⁺. ¹H NMR (400 MHz, CDCl₃) δ 7.89 (d, J=8.3 Hz, 1H), 7.55 (d, J=8.4 Hz, 1H), 7.35-7.28 (m, 2H), 7.11-7.06 (m, 1H), 6.88 (d, J=8.7 Hz, 2H), 5.92-5.84 (m, 1H), 3.54-3.29 (m, 9H), 3.14-2.97 (m, 1H), 2.89-2.77 (m, 1H), 2.65-2.46 (m, 2H), 2.24-2.11 (m, 1H).

5-nitro-4-phenoxy-2,3-dihydro-1H-inden-1-yl di(aziridin-1-yl)phosphinate

To a solution of 5-nitro-4-phenoxy-2,3-dihydro-1H-inden-1-yl di((2-bromoethyl)amino)phosphinate (30 mg, 0.055 mmol, 1.0 eq.) in THF (10 mL) were added Ag₂O (63.52 mg, 0.27 mmol, 4.9 eq.) and DIEA (0.018 mL, 0.11 mmol, 2.0 eq.). The solution was stirred at 70° C. for 12 hrs under nitrogen atmosphere. After completion, the reaction mixture was filtered and the filtrate was concentrated under reduced pressure. The residue was purified by RP-prep HPLC to give 5-nitro-4-phenoxy-2,3-dihydro-1H-inden-1-yl di(aziridin-1-yl)phosphinate (12 mg, 0.031 mmol, 56%) as a yellow oil. ¹H NMR (400 MHz, CDCl₃) δ 7.88 (d, J=8.2 Hz, 1H), 7.50 (d, J=8.2 Hz, 1H), 7.35-7.28 (m, 2H), 7.08 (t, J=7.4 Hz, 1H), 6.87 (d, J=7.8 Hz, 2H), 6.06-5.94 (m, 1H), 2.95-2.76 (m, 1H), 2.66-2.45 (m, 2H), 2.34-2.13 (m, 9H). ³¹P NMR (162 MHz, CDCl₃) δ 29.97 (s). LC-MS (ESI): m/z 402.2 [M+H]⁺.

### Illustration 4. (S)-4-(4-(dimethylcarbamoyl)phenoxy)-5-nitro-2,3-dihydro-1H-inden-1-yl di(aziridin-1-yl)phosphinate & (R)-4-(4-(dimethylcarbamoyl)phenoxy)-5-nitro-2,3-dihydro-1H-inden-1-yl di(aziridin-1-yl)phosphinate (17&18) (General Procedure 8, 6,5 core 2-method B)

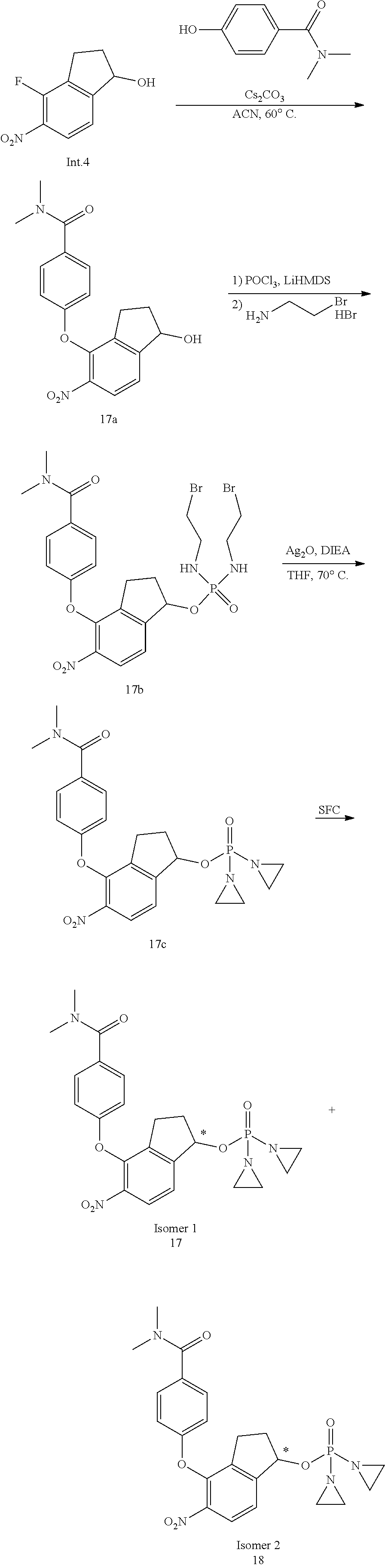

4-((1-hydroxy-5-nitro-2,3-dihydro-1H-inden-4-yl)oxy)-N,N-dimethylbenzamide

To a solution of 4-fluoro-5-nitro-2,3-dihydro-1H-inden-1-ol (180 mg, 0.91 mmol, 1.0 eq.) and 4-hydroxy-N,N-dimethylbenzamide (226 mg, 1.37 mmol, 1.5 eq.) in ACN (10 mL) was added Cs₂CO₃ (592 mg, 1.82 mmol, 2.0 eq.). The reaction mixture was stirred at 60° C. for 2 hrs. After completion, the reaction mixture was concentrated under reduced pressure. The residue was purified by Biotage® C18 column chromatography to give 4-((1-hydroxy-5-nitro-2,3-dihydro-1H-inden-4-yl)oxy)-N,N-dimethylbenzamide (120 mg, 0.35 mmol, 18%) as a gray solid. LC-MS (ESI): m/z 343 [M+H]⁺.

4-(4-(dimethylcarbamoyl)phenoxy)-5-nitro-2,3-dihydro-1H-inden-1-yl di((2-bromoethyl)amino)phosphinate

To a solution of 4-((1-hydroxy-5-nitro-2,3-dihydro-1H-inden-4-yl)oxy)-N,N-dimethylbenzamide (150 mg, 0.44 mmol, 1.0 eq.) in THF (30 mL) was added LiHMDS (0.88 mL, 1 M in THF, 0.88 mmol, 2.0 eq.) dropwise at −60° C. under N₂ and the resulting solution was stirred at −60° C. for 20 min under N₂. POCl₃ (0.082 mL, 0.89 mmol, 2.0 eq.) in THF (10 mL) was quickly added and the resulting mixture was stirred at −60° C. for 15 min. 2-Bromoethylamine hydrobromide (628 mg, 3.07 mmol, 7.0 eq.) and TEA (0.73 mL, 5.26 mmol, 12.0 eq.) were added and the mixture was stirred at −60° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 4-(4-(dimethylcarbamoyl)phenoxy)-5-nitro-2,3-dihydro-1H-inden-1-yl di((2-bromoethyl)amino)phosphinate (70 mg, 110 μmol, 25%) as a yellow oil. LC-MS (ESI): m/z 633.1 [M+H]⁺.

(S)-4-(4-(dimethylcarbamoyl)phenoxy)-5-nitro-2,3-dihydro-1H-inden-1-yl di(aziridin-1-yl)phosphinate & (R)-4-(4-(dimethylcarbamoyl)phenoxy)-5-nitro-2,3-dihydro-1H-inden-1-yl di(aziridin-1-yl)phosphinate

To a solution of 4-(4-(dimethylcarbamoyl)phenoxy)-5-nitro-2,3-dihydro-1H-inden-1-yl di((2-bromoethyl)amino)phosphinate (70 mg, 0.11 mmol, 1.0 eq.) in THF (10 mL) were added Ag₂O (256 mg, 1.10 mmol, 10.0 eq.) and DIEA (0.18 mL, 1.10 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 4-(4-(dimethylcarbamoyl)phenoxy)-5-nitro-2,3-dihydro-1H-inden-1-yl di(aziridin-1-yl)phosphinate (40 mg, 85 μmol, 77%) as a solid in a stereo isomeric mixture form, which was further separated by Chiral SFC to give:

Isomer 1(17), Retention time: 3.963 min, >99% ee. LC-MS (ESI): m/z 473.2 [M+H]⁺; ¹H NMR (400 MHz, CDCl₃) δ 7.91 (d, J=8.2 Hz, 1H), 7.54 (d, J=8.2 Hz, 1H), 7.40 (d, J=8.6 Hz, 2H), 6.88 (d, J=8.7 Hz, 2H), 6.06-5.94 (m, 1H), 3.09 (s, 3H), 3.03 (s, 3H), 2.91-2.78 (m, 1H), 2.67-2.49 (m, 2H), 2.35-2.13 (m, 9H). ³¹P NMR (162 MHz, CDCl₃) δ 29.99 (s).

Isomer 2(18), Retention time: 6.352 min, >99% ee. LC-MS (ESI): m/z 473.2 [M+H]⁺; ¹H NMR (400 MHz, CDCl₃) δ 7.91 (d, J=8.2 Hz, 1H), 7.54 (d, J=8.2 Hz, 1H), 7.40 (d, J=8.7 Hz, 2H), 6.88 (d, J=8.7 Hz, 2H), 6.09-5.93 (m, 1H), 3.09 (s, 3H), 3.03 (s, 3H), 2.90-2.79 (m, 1H), 2.67-2.51 (m, 2H), 2.36-2.09 (m, 9H). ³¹P NMR (162 MHz, CDCl₃) δ 29.99 (s).

Analytical method: Column: ChiralPak AD, 250×4.6 mm I.D., 5 μm, Mobile phase: A for CO₂ and B for ethanol (0.05% DEA), Gradient: 8 min @B 40%, Flow rate: 2.0 mL/min, Back pressure: 100 bar, Column temperature: 35° C.

SFC Method: Instrument: Waters Thar 80 preparative SFC, Column: ChiralPak AD, 250×21.2 mm I.D., 5 μm, Mobile phase: A for CO₂ and B for ETOH+0.1% NH₃H₂O, Gradient: B 35%, Flow rate: 40 mL/min, Back pressure: 100 bar, Column temperature: 35° C., Wavelength: 220 nm, Cycle-time: 10 min, Eluted time: 2.0 H.

### Illustration 5. Synthesis of (S)-4-([1,1′-biphenyl]-4-yloxy)-5-nitro-2,3-dihydro-1H-inden-1-yl di(aziridin-1-yl)phosphinate and (R)-4-([1,1′-biphenyl]-4-yloxy)-5-nitro-2,3-dihydro-1H-inden-1-yl di(aziridin-1-yl)phosphinate (19&20)

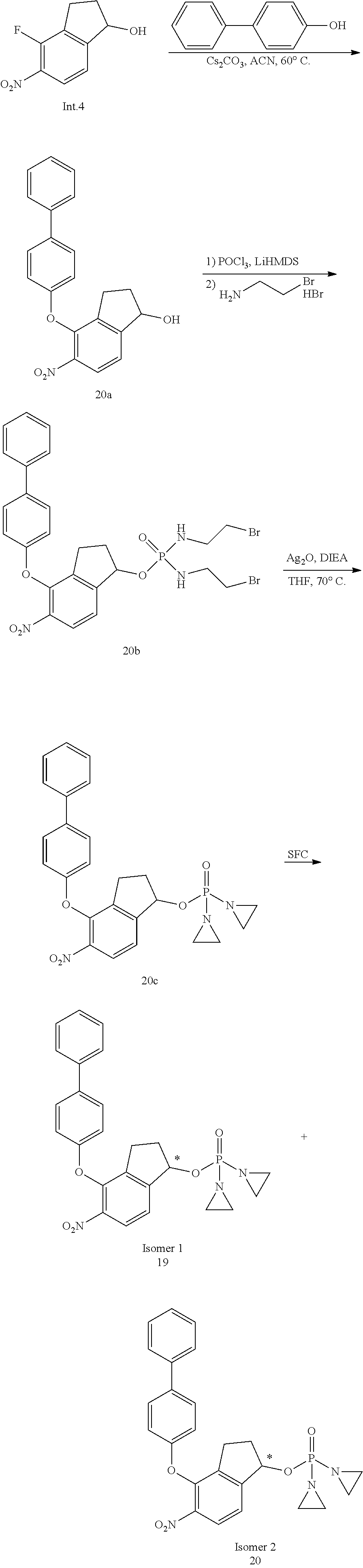

4-([1,1′-biphenyl]-4-yloxy)-5-nitro-2,3-dihydro-1H-inden-1-ol

To a solution of 4-fluoro-5-nitro-2,3-dihydro-1H-inden-1-ol (200 mg, 1.02 mmol, 1.0 eq.) and [1,1′-biphenyl]-4-ol (262 mg, 1.53 mmol, 1.5 eq.) in ACN (10 mL) was added Cs₂CO₃ (665 mg, 2.04 mmol, 2.0 eq.). The reaction mixture was stirred at 60° C. for 2 hrs. After completion, the reaction mixture was concentrated under reduced pressure. The residue was purified by Biotage® C18 column chromatography to give 5-nitro-4-(4-(pyridin-2-yl)phenoxy)-2,3-dihydro-1H-inden-1-ol (150 mg, 0.43 mmol, 42%) as a solid. LC-MS (ESI): m/z 348 [M+H]⁺.

4-([1,1′-biphenyl]-4-yloxy)-5-nitro-2,3-dihydro-1H-inden-1-yl di((2-bromoethyl)amino)phosphinate

To a solution of 5-nitro-4-(4-phenylphenoxy)-2,3-dihydro-1H-inden-1-ol (40 mg, 0.12 mmol, 1.0 eq.) in THF (15 mL) was added LiHMDS (0.23 mL, 1 M in THF, 0.23 mmol, 2.0 eq.) dropwise at −60° C. under N₂ and the resulting solution was stirred at −60° C. for 20 min under N₂. POCl₃ (153 mg, 0.23 mmol, 2.0 eq.) in THF (5 mL) was added and the resulting mixture was stirred at −60° C. for 15 min. 2-Bromoethylamine hydrobromide (423 mg, 2.07 mmol, 6.0 eq.) and TEA (412 mg, 4.08 mmol, 12.0 eq.) were added and the mixture was stirred at −60° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 4-([1,1′-biphenyl]-4-yloxy)-5-nitro-2,3-dihydro-1H-inden-1-yl di((2-bromoethyl)amino)phosphinate (20 mg, 31 μmol, 27%) as a yellow oil. LC-MS (ESI): m/z 638 [M+H]⁺.

4-([1,1′-biphenyl]-4-yloxy)-5-nitro-2,3-dihydro-1H-inden-1-yl di(aziridin-1-yl)phosphinate

To a solution of 4-([1,1′-biphenyl]-4-yloxy)-5-nitro-2,3-dihydro-1H-inden-1-yl di((2-bromoethyl)amino)phosphinate (20 mg, 31 μmol, 1.0 eq.) in THF (10 mL) were added Ag₂O (73 mg, 0.31 mmol, 10.0 eq.) and DIEA (40 mg, 0.31 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 4-([1,1′-biphenyl]-4-yloxy)-5-nitro-2,3-dihydro-1H-inden-1-yl di(aziridin-1-yl)phosphinate (3.0 mg, 6.0 μmol, 20%) as a white solid in a stereo isomeric mixture form. ¹H NMR (400 MHz, CDCl₃) δ 7.91 (d, J=8.2 Hz, 1H), 7.60-7.51 (m, 5H), 7.45-7.41 (m, 2H), 7.35-7.31 (m, 1H), 6.95-6.93 (m, 2H), 6.07-5.96 (m, 1H), 2.96-2.85 (m, 1H), 2.70-2.51 (m, 2H), 2.33-2.12 (m, 9H). ³¹P NMR (162 MHz, CDCl₃) δ 30.02 (s). LC-MS (ESI): m/z 500.2 [M+Na]—.

(S)-4-([1,1′-biphenyl]-4-yloxy)-5-nitro-2,3-dihydro-1H-inden-1-yl di(aziridin-1-yl)phosphinate and (R)-4-([1,1′-biphenyl]-4-yloxy)-5-nitro-2,3-dihydro-1H-inden-1-yl di(aziridin-1-yl)phosphinate

4-([1,1′-biphenyl]-4-yloxy)-5-nitro-2,3-dihydro-1H-inden-1-yl di(aziridin-1-yl)phosphinate (80 mg, 0.17 mmol) was further separated by Chiral SFC to give:

Isomer 1 (19), 20.1 mg, 42 μmol, 25%, Retention time: 4.803 min, >99% ee. LC-MS (ESI): m/z 500.2 [M+Na]⁺; ¹H NMR (400 MHz, CDCl₃) δ 7.90 (d, J=8.0 Hz, 1H), 7.62-7.47 (m, 5H), 7.47-7.38 (m, 2H), 7.37-7.29 (m, 1H), 6.94 (d, J=7.7 Hz, 2H), 6.10-5.94 (m, 1H), 2.97-2.84 (m, 1H), 2.73-2.51 (m, 2H), 2.37-2.12 (m, 9H). ³¹P NMR (162 MHz, CDCl₃) δ 29.99 (s).

Isomer 2 (20), 30.7 mg, 64 μmol, 38%, Retention time: 5.694 min, 99% ee. LC-MS (ESI): m/z 500.2 [M+Na]⁺; ¹H NMR (400 MHz, CDCl₃) δ 7.90 (d, J=8.0 Hz, 1H), 7.59-7.47 (m, 5H), 7.46-7.37 (m, 2H), 7.36-7.28 (m, 1H), 6.94 (d, J=7.9 Hz, 2H), 6.09-5.93 (m, 1H), 3.01-2.80 (m, 1H), 2.77-2.49 (m, 2H), 2.39-2.03 (m, 9H). ³¹P NMR (162 MHz, CDCl₃) ¿ 29.99 (s).

Analytical method: Column: ChiralPak IH, 100×4.6 mm I.D., 5 μm; Mobile phase: A for CO₂ and B for ethanol (0.05% DEA); Gradient: 8 min @ 20% B; Flow rate: 2.5 mL/min; Column temperature: 40° C.

SFC Method: Instrument: SHIMADZU PREP SOLUTION SFC, Column: ChiralPak IH, 250×21.2 mm I.D., 5 μm, Mobile phase: A for CO₂ and B for MEOH+0.1% NH₃H₂O, Gradient: B 25%, Flow rate: 40 mL/min, Back pressure: 100 bar, Column temperature: 35° C., Wavelength: 254 nm, Cycle-time: 6 min, Eluted time: 2 h.

### Illustration 6. Synthesis of (S)-4-((2,4′-difluoro-[1,1′-biphenyl]-4-yl)oxy)-5-nitro-2,3-dihydro-1H-inden-1-yl di(aziridin-1-yl)phosphinate & (R)-4-((2,4′-difluoro-[1,1′-biphenyl]-4-yl)oxy)-5-nitro-2,3-dihydro-1H-inden-1-yl di(aziridin-1-yl)phosphinate (27&28)

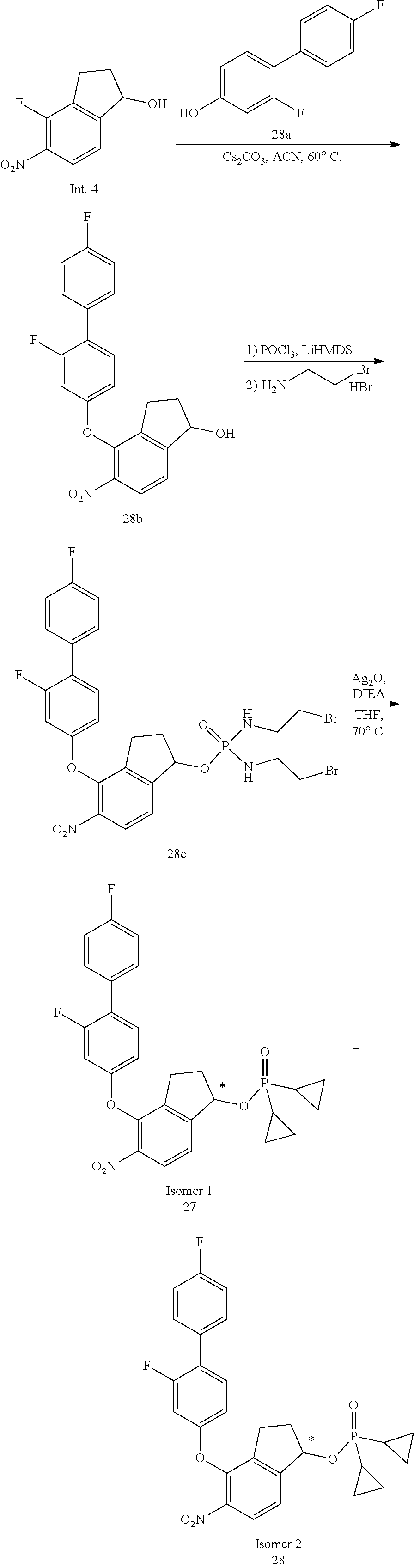

4-((2,4′-difluoro-[1,1′-biphenyl]-4-yl)oxy)-5-nitro-2,3-dihydro-1H-inden-1-ol

To a solution of 4-fluoro-5-nitro-2,3-dihydro-1H-inden-1-ol (150 mg, 0.76 mmol, 1.0 eq.) and 2,4′-difluoro-[1,1′-biphenyl]-4-ol (235 mg, 1.14 mmol, 1.5 eq.) in ACN (5 mL) was added Cs₂CO₃ (496 mg, 1.52 mmol, 2.0 eq.). The reaction mixture was stirred at 60° C. for 2 hrs. After completion, the reaction mixture was diluted with H₂O (10 mL) and extracted with EtOAc (20 mL×3). The organic layers were combined and washed with brine, dried over anhydrous Na₂SO₄ and concentrated under reduced pressure. The residue was purified by flash column chromatography on silica gel to give 4-((2,4′-difluoro-[1,1′-biphenyl]-4-yl)oxy)-5-nitro-2,3-dihydro-1H-inden-1-ol (170 mg, 0.44 mmol, 58%) as a yellow solid. LC-MS (ESI): m/z 384 [M+H]⁺.

4-((2,4′-difluoro-[1,1′-biphenyl]-4-yl)oxy)-5-nitro-2,3-dihydro-1H-inden-1-yl di((2-bromoethyl)amino)phosphinate

To a solution of 4-((2,4′-difluoro-[1,1′-biphenyl]-4-yl)oxy)-5-nitro-2,3-dihydro-1H-inden-1-ol (70 mg, 0.18 mmol, 1.0 eq.) in THF (10 mL) was added LiHMDS (0.22 mL, 1 M in THF, 0.22 mmol, 1.2 eq.) dropwise at −78° C. under N₂ and the resulting solution was stirred at −78° C. for 15 min. POCl₃ (55 mg, 0.37 mmol, 2.0 eq.) in THF (5 mL) was added and the resulting mixture was stirred at −78° C. for 15 min. 2-Bromoethylamine hydrobromide (221 mg, 1.08 mmol, 6.0 eq.) and TEA (218 mg, 2.16 mmol, 12.0 eq.) were added. The resulting mixture was warmed up to room temperature and stirred for 1 hr. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 4-((2,4′-difluoro-[1,1′-biphenyl]-4-yl)oxy)-5-nitro-2,3-dihydro-1H-inden-1-yl di((2-bromoethyl)amino)phosphinate (50 mg, 74 μmol, 41%) as a yellow oil. LC-MS (ESI): m/z 696 [M+Na]⁺.

4-((2,4′-difluoro-[1,1′-biphenyl]-4-yl)oxy)-5-nitro-2,3-dihydro-1H-inden-1-yl di(aziridin-1-yl)phosphinate

To a solution of 4-((2,4′-difluoro-[1,1′-biphenyl]-4-yl)oxy)-5-nitro-2,3-dihydro-1H-inden-1-yl di((2-bromoethyl)amino)phosphinate (100 mg, 0.15 mmol, 1.0 eq.) in THF (5 mL) were added Ag₂O (206 mg, 0.89 mmol, 6.0 eq.) and DIEA (115 mg, 0.89 mmol, 6.0 eq.). The resulting solution was stirred at 70° C. for 18 hrs under N₂ After completion, the reaction mixture was filtered and the filtrate was concentrated under reduced pressure. The residue was purified by Biotage® C18 column chromatography to afford 4-((2,4′-difluoro-[1,1′-biphenyl]-4-yl)oxy)-5-nitro-2,3-dihydro-1H-inden-1-yl di(aziridin-1-yl)phosphinate (30 mg, 58 μmol, 39%) as a white solid in a stereo isomeric mixture form. ¹H NMR (400 MHz, CDCl₃) δ 7.94 (d, J=8.2 Hz, 1H), 7.57 (d, J=8.2 Hz, 1H), 7.51-7.43 (m, 2H), 7.32 (t, J=8.6 Hz, 1H), 7.17-7.06 (m, 2H), 6.75-6.66 (m, 2H), 6.07-5.99 (m, 1H), 3.00-2.90 (m, 1H), 2.76-2.56 (m, 2H), 2.31-2.16 (m, 9H). ³¹P NMR (162 MHz, CDCl₃) δ 30.02 (s). ¹⁹F NMR (376 MHz, CDCl₃) δ −114.53 (s), −114.69 (s). LC-MS (ESI): m/z 536.1 [M+Na]⁺.

(S)-4-((2,4′-difluoro-[1,1′-biphenyl]-4-yl)oxy)-5-nitro-2,3-dihydro-1H-inden-1-yl di(aziridin-1-yl)phosphinate and (R)-4-((2,4′-difluoro-[1,1′-biphenyl]-4-yl)oxy)-5-nitro-2,3-dihydro-1H-inden-1-yl di(aziridin-1-yl)phosphinate

4-((2,4′-difluoro-[1,1′-biphenyl]-4-yl)oxy)-5-nitro-2,3-dihydro-1H-inden-1-yl di(aziridin-1-yl)phosphinate (50 mg, 97.5 μmol) was further separated by Chiral SFC to give:

Isomer 1 (27), 16.4 mg, 32 μmol, 33%, Retention time: 2.872 min, 99% ee. LC-MS (EST): m/z=536.1 [M+Na]⁺; ¹H NMR (400 MHz, CDCl₃) δ 7.93 (d, J=8.2 Hz, 1H), 7.56 (d, J=8.2 Hz, 1H), 7.49-7.43 (m, 2H), 7.32 (t, J=8.6 Hz, 1H), 7.11 (t, J=8.7 Hz, 2H), 6.76-6.65 (m, 2H), 6.07-5.99 (m, 1H), 3.01-2.90 (m, 1H), 2.75-2.56 (m, 2H), 2.36-2.13 (m, 9H). ³¹P NMR (162 MHz, CDCl₃) δ 30.02 (s). ¹⁹F NMR (376 MHz, CDCl₃) δ −114.52 (s), −114.69 (s).

Isomer 2 (28), 18.4 mg, 35 μmol, 37%, Retention time: 4.362 min, 99% ee. LC-MS (ESI): m/z 536.1 [M+Na]⁺; ¹H NMR (400 MHz, CDCl₃) δ 7.93 (d, J=8.2 Hz, 1H), 7.56 (d, J=8.2 Hz, 1H), 7.50-7.42 (m, 2H), 7.32 (t, J=8.6 Hz, 1H), 7.15-7.08 (m, 2H), 6.75-6.66 (m, 2H), 6.06-5.99 (m, 1H), 3.00-2.90 (m, 1H), 2.75-2.56 (m, 2H), 2.34-2.14 (m, 9H). ³¹P NMR (162 MHz, CDCl₃) δ 30.02 (s). ¹⁹F NMR (376 MHz, CDCl₃) δ −114.52 (s), −114.69 (s).

Analytical method: Column: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

SFC Method: Instrument: Waters Thar 80 preparative SFC, Column: ChiralPak C-IG, 250×21.2 mm I.D., 5 μm, Mobile phase: A for CO₂ and B for MEOH+0.1% NH₃H₂O, Gradient: B 40%, Flow rate: 40 mL/min, Back pressure: 100 bar, Column temperature: 35° C., Wavelength: 220 nm, Cycle-time: 20 min, Eluted time: 2 H.

### Illustration 7. Synthesis of (S)-4-([1,1′-biphenyl]-3-yloxy)-5-nitro-2,3-dihydro-1H-inden-1-yl di(aziridin-1-yl)phosphinate & (R)-4-([1,1′-biphenyl]-3-yloxy)-5-nitro-2,3-dihydro-1H-inden-1-yl di(aziridin-1-yl)phosphinate (30&31)

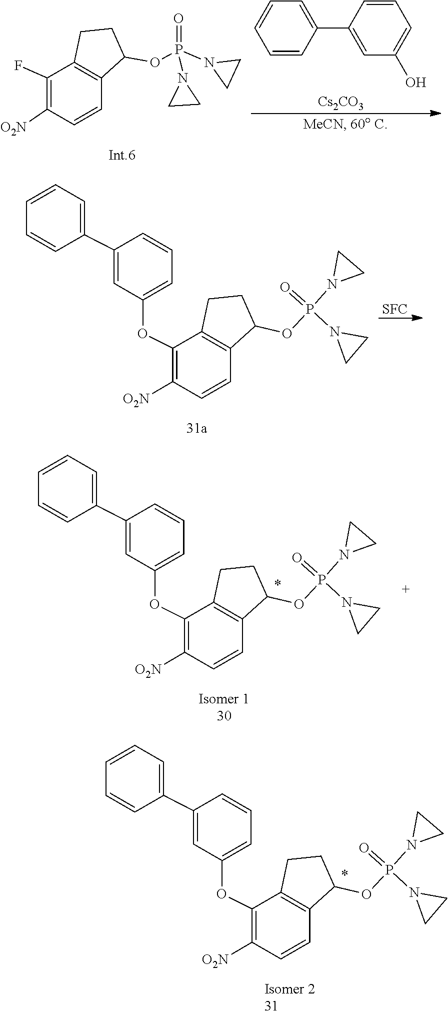

4-([1,1′-biphenyl]-3-yloxy)-5-nitro-2,3-dihydro-1H-inden-1-yl di(aziridin-1-yl)phosphinate

To a solution of 4-fluoro-5-nitro-2,3-dihydro-1H-inden-1-yl di(aziridin-1-yl)phosphinate (20 mg, 61.2 μmol, 1.0 eq.) and [1,1′-biphenyl]-3-ol (15.6 mg, 91.8 μmol, 1.5 eq.) in MeCN (5 mL) was added Cs₂CO₃ (39.9 mg, 122.4 μmol, 2.0 eq.). The reaction mixture was stirred at 60° C. for 2 hrs. After completion, the reaction mixture was concentrated under reduced pressure. The residue was purified by Biotage® C18 column chromatography to give 4-([1,1′-biphenyl]-3-yloxy)-5-nitro-2,3-dihydro-1H-inden-1-yl di(aziridin-1-yl)phosphinate (4.7 mg, 9.85 μmol, 16%) as a white solid in a stereo isomeric mixture form. ¹H NMR (400 MHz, CDCl₃) δ 7.90 (d, J=8.2 Hz, 1H), 7.56-7.50 (m, 3H), 7.46-7.39 (m, 2H), 7.39-7.29 (m, 3H), 7.12-7.09 (m, 1H), 6.85-6.80 (m, 1H), 6.04-5.96 (m, 1H), 2.95-2.85 (m, 1H), 2.69-2.49 (m, 2H), 2.28-2.18 (m, 9H). ³¹P NMR (162 MHz, CDCl₃) δ 29.90 (s). LC-MS (ESI): m/z 500.2 [M+Na]⁺.

(S)-4-([1,1′-biphenyl]-3-yloxy)-5-nitro-2,3-dihydro-1H-inden-1-yl di(aziridin-1-yl)phosphinate & (R)-4-([1,1′-biphenyl]-3-yloxy)-5-nitro-2,3-dihydro-1H-inden-1-yl di(aziridin-1-yl)phosphinate
4-([1,1′-biphenyl]-3-yloxy)-5-nitro-2,3-dihydro-1H-inden-1-yl di(aziridin-1-yl)phosphinate (75 mg, 0.16 mmol) was purified by Chiral SFC to give:

Isomer 1 (30), 33.9 mg, 71 μmol, 45%, Retention time: 2.148 min, >99% ee. LC-MS (ESI): m/z 478.2 [M+H]⁺; ¹H NMR (400 MHz, CDCl₃) δ 7.82 (d, J=8.2 Hz, 1H), 7.49-7.41 (m, 3H), 7.39-7.32 (m, 2H), 7.31-7.21 (m, 3H), 7.05-7.01 (m, 1H), 6.79-6.72 (m, 1H), 5.97-5.88 (m, 1H), 2.88-2.76 (m, 1H), 2.61-2.43 (m, 2H), 2.21-2.08 (m, 9H). ³¹P NMR (162 MHz, CDCl₃) δ 29.90 (s).

Isomer 2 (31), 34.9 mg, 73 μmol, 47%, Retention time: 2.544 min, 99% ee. LC-MS (ESI): m/z 478.2 [M+H]⁺; ¹H NMR (400 MHz, CDCl₃) δ 7.82 (d, J=8.2 Hz, 1H), 7.49-7.41 (m, 3H), 7.39-7.32 (m, 2H), 7.30-7.21 (m, 3H), 7.07-7.01 (m, 1H), 6.78-6.72 (m, 1H), 5.95-5.88 (m, 1H), 2.87-2.77 (m, 1H), 2.62-2.42 (m, 2H), 2.22-2.08 (m, 8H). ³¹P NMR (162 MHz, CDCl₃) δ 29.90 (s).

Analytical method: Column: ChiralPak IB, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

SFC Method: Instrument: Waters Thar 80 preparative SFC, Column: ChiralPak IB, 250×21.2 mm I.D., 5 μm, Mobile phase: A for CO₂ and B for MEOH+0.1% NH₃H₂O, Gradient: B 27%, Flow rate: 40 mL/min, Back pressure: 100 bar, Column temperature: 35° C., Wavelength: 220 nm, Cycle-time: 15 min, Eluted time: 2.0 H.

### Illustration 8. Synthesis of (R)-4-(3-(dimethylcarbamoyl)phenoxy)-5-nitro-2,3-dihydro-1H-inden-1-yl di(aziridin-1-yl)phosphinate & (S)-4-(3-(dimethylcarbamoyl)phenoxy)-5-nitro-2,3-dihydro-1H-inden-1-yl di(aziridin-1-yl)phosphinate (32&33)

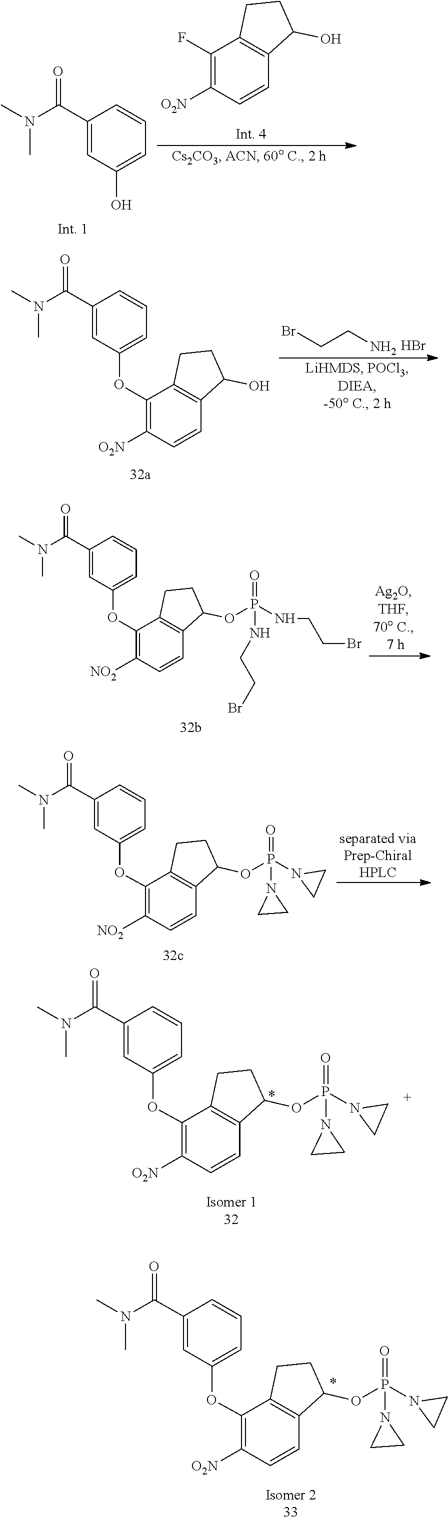

3-[(1-hydroxy-5-nitro-2,3-dihydro-1H-inden-4-yl)oxy]-N,N-dimethylbenzamide

A mixture of 4-fluoro-5-nitro-2,3-dihydro-1H-inden-1-ol (910 mg, 4.62 mmol, 1.00 equiv), 3-hydroxy-N, N-dimethylbenzamide (840 mg, 5.09 mmol, 1.10 equiv) and Cs₂CO₃ (3.00 g, 9.23 mmol, 2.00 equiv) in MeCN (10.0 mL) was stirred for 2 h at 60° C. The mixture was allowed to cool down to room temperature. The reaction was quenched with water at room temperature. The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford 3-[(1-hydroxy-5-nitro-2,3-dihydro-1H-inden-4-yl)oxy]-N,N-dimethylbenzamide (960 mg, 60.8%) as a yellow solid. LC-MS (ESI): m/z 343.20 [M+H]⁺

4-[3-(dimethylcarbamoyl)phenoxy]-5-nitro-2,3-dihydro-1H-inden-1-yl di((2-bromoethyl)amino)phosphinate

To a stirred solution of 3-[(1-hydroxy-5-nitro-2,3-dihydro-1H-inden-4-yl)oxy]-N,N-dimethylbenzamide (830 mg, 2.42 mmol, 1.00 equiv) in THF (170 mL) was added LiHMDS (6.10 mL, 6.06 mmol, 2.50 equiv, 1.0 mol/L in THF) dropwise at −50° C. under nitrogen atmosphere. The resulting mixture was stirred for 20 min at −50° C. under nitrogen atmosphere. To the above mixture was added POCl₃ (929 mg, 6.06 mmol, 2.50 equiv) dropwise at −50° C. The resulting mixture was stirred for additional 20 min at −50° C. To the above mixture was added 2-bromoethan-1-amine hydrobromide (3.0 g, 14.5 mmol, 6.00 equiv) and DIEA (2.5 g, 19.4 mmol, 8.00 equiv) at −50° C. The resulting mixture was stirred for additional 30 min at −50° C. and then stirred for 1 h at room temperature under nitrogen atmosphere. The reaction was quenched with water at room temperature. The resulting mixture was extracted with CH₂Cl₂ (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with CH₂Cl₂/MeOH (10:1) to afford 4-[3-(dimethylcarbamoyl)phenoxy]-5-nitro-2,3-dihydro-1H-inden-1-yl di((2-bromoethyl)amino)phosphinate (929 mg, 60.4%) as a yellow solid. LC-MS (ESI): m/z 633.10, 635.10, 637.05 [M+H]⁺

4-[3-(dimethylcarbamoyl)phenoxy]-5-nitro-2,3-dihydro-1H-inden-1-yl bis(aziridin-1-yl)phosphinate

A mixture of 4-[3-(dimethylcarbamoyl)phenoxy]-5-nitro-2,3-dihydro-1H-inden-1-yl di((2-bromoethyl)amino)phosphinate (500 mg, 0.79 mmol, 1.00 equiv), Ag₂O (895 mg, 3.86 mmol, 4.9 equiv) and DIEA (204 mg, 1.58 mmol, 2.00 equiv) in THF (166 mL) was stirred for 7 h at 70° C. under nitrogen atmosphere. The mixture was allowed to cool down to room temperature. The resulting mixture was filtered, the filter cake was washed with CH₂Cl₂ (3×50 mL). The filtrate was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 30% B to 35% B in 7 min, 35% B; Wave Length: 254/220 nm;) to afford 4-[3-(dimethylcarbamoyl)phenoxy]-5-nitro-2,3-dihydro-1H-inden-1-yl bis(aziridin-1-yl)phosphinate (237 mg, 63.6%) as an off-white oil. LC-MS (ESI): m/z 473.25 [M+H]⁺

(R)-4-(3-(dimethylcarbamoyl)phenoxy)-5-nitro-2,3-dihydro-1H-inden-1-yl di(aziridin-1-yl)phosphinate & (S)-4-(3-(dimethylcarbamoyl)phenoxy)-5-nitro-2,3-dihydro-1H-inden-1-yl di(aziridin-1-yl)phosphinate

The compound 4-[3-(dimethylcarbamoyl)phenoxy]-5-nitro-2,3-dihydro-1H-inden-1-ylbis(aziridin-1-yl)phosphinate (237 mg) was separated by Prep-Chiral HPLC with the following conditions: Column: CHIRAL ART Cellulose-SZ, 3*25 cm, 5 μm; Mobile Phase A: Hex (0.5% 2M NH₃-MeOH)—HPLC, Mobile Phase B: IPA--HPLC; Flow rate: 40 mL/min; Gradient: 50% B to 50% B in 34 min; Wave Length: 200/220 nm; RT1 (min): 21.5; RT2 (min): 27.5; Sample Solvent: MeOH: DCM=1:1; InJection Volume: 0.4 mL; Number Of Runs: 14.

Isomer 1 (32), 40.7 mg, 17.2%, ee>99.0%, LC-MS (ESI): m/z 473.25 [M+H]⁺ ¹H NMR (400 MHz, DMSO-d₆) δ 8.04 (d, J=8.2 Hz, 1H), 7.55 (d, J=8.2 Hz, 1H), 7.43 (t, J=7.9 Hz, 1H), 7.13 (dt, J=7.6, 1.2 Hz, 1H), 7.01 (ddd, J=8.3, 2.7, 1.0 Hz, 1H), 6.91-6.82 (m, 1H), 5.94 (q, J=6.7 Hz, 1H), 2.94 (s, 3H), 2.85 (s, 3H), 2.79-2.66 (m, 1H), 2.61-2.52 (m, 2H), 2.20-2.01 (m, 9H). ³¹P NMR (162 MHz, DMSO-d₆) δ 30.10 (d, J=2.8 Hz).

Isomer 2 (33), 49.1 mg, 20.7, ee>99.0%, LC-MS (ESI): m/z 473.25 [M+H]^{+ 1}H NMR (400 MHz, DMSO-d₆) δ 8.04 (d, J=8.2 Hz, 1H), 7.55 (d, J=8.2 Hz, 1H), 7.43 (t, J=7.9 Hz, 1H), 7.13 (dt, J=7.6, 1.3 Hz, 1H), 7.01 (dd, J=7.9, 2.7 Hz, 1H), 6.91-6.85 (m, 1H), 5.94 (q, J=6.5 Hz, 1H), 2.94 (s, 3H), 2.85 (s, 3H), 2.78-2.66 (m, 1H), 2.62-2.52 (m, 2H), 2.26-1.98 (m, 9H). ³¹P NMR (162 MHz, DMSO-d₆) δ 30.10.

### Illustration 9 Synthesis of (R)-4-((2-cyclopropyl-3-oxoisoindolin-5-yl)oxy)-5-nitro-2,3-dihydro-1H-inden-1-yl di(aziridin-1-yl)phosphinate & (S)-4-((2-cyclopropyl-3-oxoisoindolin-5-yl)oxy)-5-nitro-2,3-dihydro-1H-inden-1-yl di(aziridin-1-yl)phosphinate (38&39)

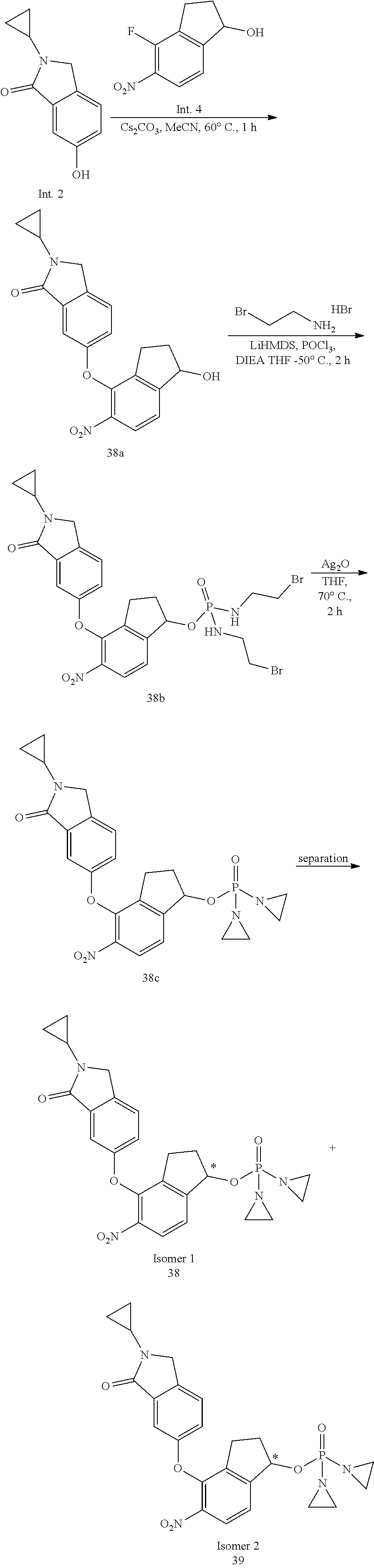

2-cyclopropyl-6-[(1-hydroxy-5-nitro-2,3-dihydro-1H-inden-4-yl)oxy]-3H-isoindol-1-one

To a stirred solution 4-fluoro-5-nitro-2,3-dihydro-1H-inden-1-ol (500 mg, 2.54 mmol, 1.00 equiv) and 2-cyclopropyl-6-hydroxy-3H-isoindol-1-one (528 mg, 2.79 mmol, 1.10 equiv) in MeCN (15.0 mL) was added Cs₂CO₃ (1.65 g, 5.07 mmol, 2.00 equiv). The resulting mixture was stirred for 1 h at 60° C. The reaction was quenched with water (100 mL) and extracted with EtOAc (3×10 0 mL). The combined organic layers were washed with brine (2×30 mL), dried over anhydrous na₂SO₄. After filtration, the filtrate was concentrated under reduced pressure. The residue was purified by silica gel column chromatography, eluted with PE/EA (1:1) to afford 2-cyclopropyl-6-[(1-hydroxy-5-nitro-2,3-dihydro-1H-inden-4-yl)oxy]-3H-isoindol-1-one (340 mg, 36.5%) as a brown solid. LC-MS (ESI): m/z 367.15 [M+H]⁺

4-[(2-cyclopropyl-3-oxo-1H-isoindol-5-yl)oxy]-5-nitro-2,3-dihydro-1H-inden-1-yl di((2-bromoethyl)amino)phosphinate

To a stirred solution 2-cyclopropyl-6-[(1-hydroxy-5-nitro-2,3-dihydro-1H-inden-4-yl)oxy]-3H-isoindol-1-one (500 mg, 1.36 mmol, 1.00 equiv) in THF (65.0 mL) was added LiHMDS (2.05 mL, 2.05 mmol, 1.0 M in THF, 1.50 equiv) dropwise at −50° C. under nitrogen atmosphere. The mixture was stirred for additional 20 min at −50° C. To the above mixture was added POCl₃ (314 mg, 2.05 mmol, 1.50 equiv) dropwise. The resulting mixture was stirred for additional 20 min at −50° C. Then to the above mixture was added 2-bromoethanamine hydrobromide (1.68 g, 8.19 mmol, 6.00 equiv) and DIEA (1.41 g, 10.9 mmol, 8.00 equiv). The resulting mixture was stirred for additional 2 h at room temperature. The reaction was quenched by the addition of water (30 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase A: Water (0.1% NH₃·H₂O), mobile phase B: MeCN, 10% to 90% gradient in 30 min; detector, UV 254 nm. To afford 4-[(2-cyclopropyl-3-oxo-1H-isoindol-5-yl)oxy]-5-nitro-2,3-dihydro-1H-inden-1-yl di((2-bromoethyl)amino)phosphinate (690 mg, 76.8%) as a brown oil. LC-MS (ESI): m/z 657.00, 659.00, 660.95 [M+H]⁺

4-[(2-cyclopropyl-3-oxo-1H-isoindol-5-yl)oxy]-5-nitro-2,3-dihydro-1H-inden-1-yl bis(aziridin-1-yl)phosphinate

To a stirred solution 4-[(2-cyclopropyl-3-oxo-1H-isoindol-5-yl)oxy]-5-nitro-2,3-dihydro-1H-inden-1-yl di((2-bromoethyl)amino)phosphinate (650 mg, 0.99 mmol, 1.00 equiv) and Ag₂O (1.14 g, 4.93 mmol, 5.00 equiv) in THF (10.0 mL) was added DIEA (638 mg, 4.94 mmol, 5.00 equiv) at room temperature under nitrogen atmosphere. The resulting mixture was stirred for 2 h at 70° C. The resulting mixture was filtered, the filter cake was washed with EtOAc (3×30 mL). The filtrate was concentrated under reduced pressure. The residue was purified by Prep-HPLC with the following conditions (Column: XB C18, Mobile Phase A: Water (10 mmol/L NH₃ H₂O), Mobile Phase B: ACN; Flow rate: 40 mL/min; Gradient: 15% B to 55% B in 40 min; Wave Length: 254/220 nm). This resulted in 4-[(2-cyclopropyl-3-oxo-1H-isoindol-5-yl)oxy]-5-nitro-2,3-dihydro-1H-inden-1-yl bis(aziridin-1-yl)phosphinate (357 mg, 72.8%) as a white solid. LC-MS (ESI): m/z 497.25 [M+H]⁺

(R)-4-((2-cyclopropyl-3-oxoisoindolin-5-yl)oxy)-5-nitro-2,3-dihydro-1H-inden-1-yl di(aziridin-1-yl)phosphinate & (S)-4-((2-cyclopropyl-3-oxoisoindolin-5-yl)oxy)-5-nitro-2,3-dihydro-1H-inden-1-yl di(aziridin-1-yl)phosphinate

The compound 4-[(2-cyclopropyl-3-oxo-1H-isoindol-5-yl)oxy]-5-nitro-2,3-dihydro-1H-inden-1-yl bis(aziridin-1-yl)phosphinate was separated by Chiral HPLC with the following conditions: CHIRALPAK IE, 3*25 cm, 5 μm; Mobile Phase A: HEX: MtBE=1: 1(1: 1 (0.5% 2 M NH₃-MEOH), Mobile Phase B: MeOH—HPLC; Flow rate: 40 mL/min; Gradient: 30% B to 30% B in 22 min; Wave Length: 212/260 nm; RT1 (min): 16.7; RT2 (min): 19.2; Sample Solvent: MeOH: DCM=1: 1-HPLC; InJection Volume: 0.6 mL; Number Of Runs: 11.

Isomer 1 (38), 107.4 mg, 22.25%, ee>99%, LC-MS (ESI): m/z 497.25 [M+H]^{+ 1}H NMR (300 MHz, DMSO-d₆) δ 8.06 (d, J=8.2 Hz, 1H), 7.68-7.48 (m, 2H), 7.26 (dd, J=8.3, 2.5 Hz, 1H), 6.95 (d, J=2.5 Hz, 1H), 5.96 (q, J=6.5, 6.0 Hz, 1H), 4.37 (s, 2H), 3.02-2.84 (m, 1H), 2.83-2.62 (m, 1H), 2.61-2.51 (m, 2H), 2.29-1.88 (m, 9H), 0.93-0.68 (m, 4H). ³¹P NMR (121 MHz, DMSO-d₆) δ 30.09.

Isomer 2 (39), 88.6 mg, 18.36%, ee>99%, LC-MS (ESI): m/z 497.25 [M+H]+1H NMR (300 MHz, DMSO-d₆) δ 8.06 (d, J=8.2 Hz, 1H), 7.64-7.52 (m, 2H), 7.27 (dd, J=8.3, 2.5 Hz, 1H), 6.95 (d, J=2.4 Hz, 1H), 6.03-5.90 (m, 1H), 4.37 (s, 2H), 3.00-2.85 (m, 1H), 2.81-2.65 (m, 1H), 2.61-2.52 (m, 2H), 2.23-1.97 (m, 9H), 0.93-0.71 (m, 4H). ³¹P NMR (121 MHz, DMSO-d₆) δ 30.09.

### Illustration 10. Synthesis of (S)-5-nitro-4-(4-(pyridin-2-yl)phenoxy)-2,3-dihydro-1H-inden-1-yl di(aziridin-1-yl)phosphinate and (R)-5-nitro-4-(4-(pyridin-2-yl)phenoxy)-2,3-dihydro-1H-inden-1-yl di(aziridin-1-yl)phosphinate (40&41)

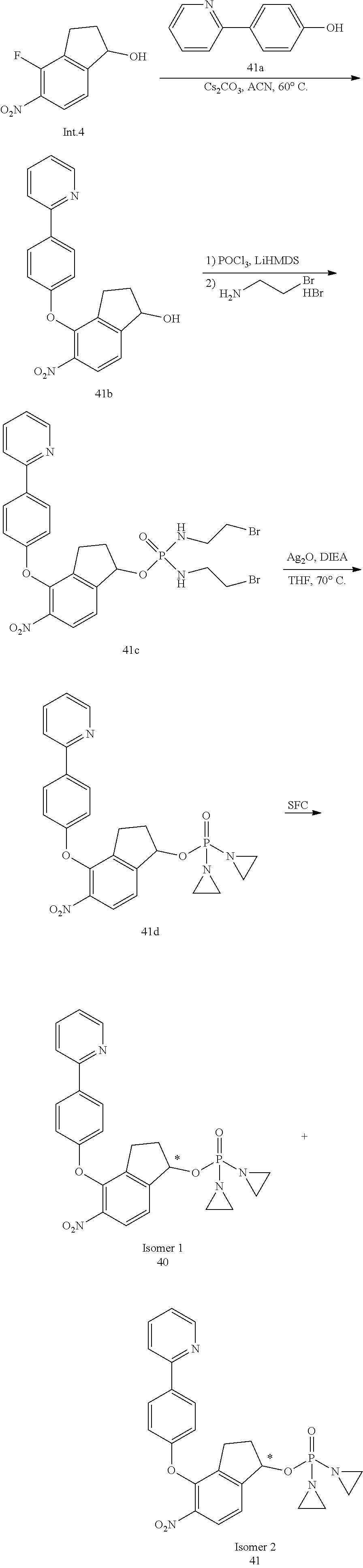

5-nitro-4-(4-(pyridin-2-yl)phenoxy)-2,3-dihydro-1H-inden-1-ol

To a solution of 4-fluoro-5-nitro-2,3-dihydro-1H-inden-1-ol (200 mg, 1.02 mmol, 1.0 eq.) and 4-(pyridin-2-yl)phenol (262 mg, 1.53 mmol, 1.5 eq.) in ACN (10 mL) was added Cs₂CO₃ (665 mg, 2.04 mmol, 2.0 eq.). The reaction mixture was stirred at 60° C. for 2 hrs. After completion, the reaction mixture was concentrated under reduced pressure. The residue was purified by Biotage® C18 column chromatography to give 5-nitro-4-(4-(pyridin-2-yl)phenoxy)-2,3-dihydro-1H-inden-1-ol (3, 150 mg, 0.43 mmol, 42%) as a gray solid. LC-MS (ESI): m/z 349 [M+H]⁺.

5-nitro-4-(4-(pyridin-2-yl)phenoxy)-2,3-dihydro-1H-inden-1-yl di((2-bromoethyl)amino)phosphinate

To a solution of 5-nitro-4-(4-(pyridin-2-yl)phenoxy)-2,3-dihydro-1H-inden-1-ol (120 mg, 0.34 mmol, 1.0 eq.) in THF (15 mL) was added LiHMDS (0.68 mL, 1 M in THF, 0.68 mmol, 2.0 eq.) dropwise at −60° C. under N₂ and the resulting solution was stirred at −60° C. for 20 min under N₂. POCl₃ (104 mg, 0.68 mmol, 2.0 eq.) in THF (5 mL) was added and the resulting mixture was stirred at −60° C. for 15 min. 2-Bromoethylamine hydrobromide (423 mg, 2.07 mmol, 6.0 eq.) and TEA (412 mg, 4.08 mmol, 12.0 eq.) were added and the mixture was stirred at −60° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 5-nitro-4-(4-(pyridin-2-yl)phenoxy)-2,3-dihydro-1H-inden-1-yl di((2-bromoethyl)amino)phosphinate (170 mg, 0.27 mmol, 77%) as a yellow oil. LC-MS (ESI): m/z 639 [M+H]⁺.

5-nitro-4-(4-(pyridin-2-yl)phenoxy)-2,3-dihydro-1H-inden-1-yl di(aziridin-1-yl)phosphinate

To a solution of 5-nitro-4-(4-(pyridin-2-yl)phenoxy)-2,3-dihydro-1H-inden-1-yl di((2-bromoethyl)amino)phosphinate (210 mg, 0.33 mmol, 1.0 eq.) in THF (10 mL) were added Ag₂O (766 mg, 3.30 mmol, 10.0 eq.) and DIEA (426 mg, 3.30 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 5-nitro-4-(4-(pyridin-2-yl)phenoxy)-2,3-dihydro-1H-inden-1-yl di(aziridin-1-yl)phosphinate 126 mg, 0.26 mmol, 80%) as a yellow oil in a stereo isomeric mixture form. ¹H NMR (400 MHz, CDCl₃) δ 8.79-8.59 (m, 1H), 8.00-7.87 (m, 3H), 7.83-7.62 (m, 2H), 7.59-7.50 (m, 1H), 7.25-7.16 (m, 1H), 7.05-6.86 (m, 2H), 6.15-5.74 (m, 1H), 2.98-2.82 (m, 1H), 2.73-2.48 (m, 2H), 2.37-2.09 (m, 9H). ³¹P NMR (162 MHz, CDCl₃) δ 29.92 (s). LC-MS (ESI): m/z 479.1 [M+H]⁺.

(S)-5-nitro-4-(4-(pyridin-2-yl)phenoxy)-2,3-dihydro-1H-inden-1-yl di(aziridin-1-yl)phosphinate & (R)-5-nitro-4-(4-(pyridin-2-yl)phenoxy)-2,3-dihydro-1H-inden-1-yl di(aziridin-1-yl)phosphinate

5-nitro-4-(4-(pyridin-2-yl)phenoxy)-2,3-dihydro-1H-inden-1-yl di(aziridin-1-yl)phosphinate (126 mg, 0.26 mmol) was further separated by Chiral SFC to give:

Isomer 1(40), 50 mg, 0.11 mmol, 32%, Retention time: 1.941 min, >99% ee. LC-MS (ESI): m/z 479.1 [M+H]⁺; ¹H NMR (400 MHz, CDCl₃) δ 8.58 (d, J=4.6 Hz, 1H), 7.92-7.79 (m, 3H), 7.71-7.56 (m, 2H), 7.45 (d, J=8.2 Hz, 1H), 7.16-7.10 (m, 1H), 6.93-6.85 (m, 2H), 6.00-5.85 (m, 1H), 2.91-2.69 (m, 1H), 2.61-2.41 (m, 2H), 2.21-2.10 (m, 9H). ³¹P NMR (162 MHz, CDCl₃) δ 29.90 (s).

Isomer 2(41), 50 mg, 0.11 mmol, 32%, Retention time: 2.317 min, 95% ee. LC-MS (ESI): m/z 479.1 [M+H]⁺; ¹H NMR (400 MHz, CDCl₃) δ 8.67 (d, J=4.6 Hz, 1H), 7.99-7.86 (m, 3H), 7.78-7.71 (m, 1H), 7.71-7.64 (m, 1H), 7.54 (d, J=8.2 Hz, 1H), 7.26-7.19 (m, 1H), 7.05-6.91 (m, 2H), 6.10-5.89 (m, 1H), 2.95-2.83 (m, 1H), 2.70-2.49 (m, 2H), 2.32-2.18 (m, 9H). ³¹P NMR (162 MHz, CDCl₃) δ 29.91 (s).

Analytical method: Column: ChiralPak IH, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

SFC Method: Instrument: Waters Thar 80 preparative SFC, Column: ChiralPak AD, 250×21.2 mm I.D., 5 μm, Mobile phase: A for CO₂ and B for MEOH+0.1% NH₃H₂O, Gradient: B 30%, Flow rate: 40 mL/min, Back pressure: 100 bar, Column temperature: 35° C., Wavelength: 220 nm, Cycle-time: 10 min, Eluted time: 2 H.

### Illustration 11. Synthesis of (S)-4-([1,1′-biphenyl]-3-yloxy)-5-nitro-2,3-dihydro-1H-inden-1-yl di(aziridin-1-yl)phosphinate & (R)-4-([1,1′-biphenyl]-3-yloxy)-5-nitro-2,3-dihydro-1H-inden-1-yl di(aziridin-1-yl)phosphinate (42&43)

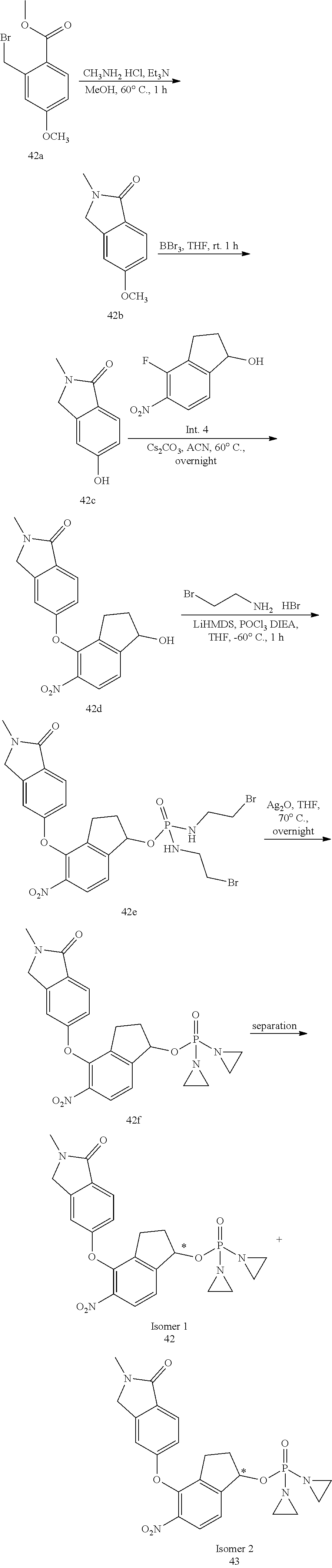

5-methoxy-2-methyl-3H-isoindol-1-one

To a stirred solution of methyl 2-(bromomethyl)-4-methoxybenzoate (3.00 g, 11.6 mmol, 1.00 equiv) and CH₃NH₂HCl (1.56 g, 23.2 mmol, 2.00 equiv) in MeOH (30.0 mL) were added Et₃N (3.52 g, 34.7 mmol, 3.00 equiv). The resulting mixture was stirred for 1 h at 60° C. The resulting mixture was quenched with water at room temperature, and extracted with CH₂Cl₂ (3×100 mL). The combined organic layers were dried over anhydrous Na₂SO₄. After filtration, the filtrate was concentrated under reduced pressure. The residue was purified by silica gel column chromatography, eluted with PE/EA (1:1) to afford 5-methoxy-2-methyl-3H-isoindol-1-one (1.15 g, 56.0%) as a white solid. LC-MS (ESI): m/z 178.10 [M+H]⁺5-hydroxy-2-methyl-3H-isoindol-1-one

To a stirred solution of 5-methoxy-2-methyl-3H-isoindol-1-one (1.42 g, 8.01 mmol, 1.00 equiv) in THF (150 mL) was added BBr₃ (24.0 mL, 24.0 mmol, 3.00 equiv, 1.0 mol/L in DCM dropwise at 0° C. under nitrogen atmosphere. The resulting mixture was stirred for 1 h at room temperature under nitrogen atmosphere. The reaction was quenched by the addition of Water (100 mL) at 0° C. The resulting mixture was extracted with CH₂Cl₂ (3× 100 mL). The combined organic layers were dried over anhydrous Na₂SO₄. After filtration, the filtrate was concentrated under reduced pressure. The residue was purified by silica gel column chromatography, eluted with PE/EA (3:1) to afford 5-hydroxy-2-methyl-3H-isoindol-1-one (1.02 g, 78.0%) as a white solid. LC-MS (ESI): m/z 164.10 [M+H]⁺
5-[(1-hydroxy-5-nitro-2,3-dihydro-1H-inden-4-yl)oxy]-2-methyl-3H-isoindol-1-one

A mixture of 4-fluoro-5-nitro-2,3-dihydro-1H-inden-1-ol (700 mg, 3.55 mmol, 1.00 equiv), Cs₂CO₃ (2.31 g, 7.10 mmol, 2.00 equiv) and 5-hydroxy-2-methyl-3H-isoindol-1-one (579 mg, 3.55 mmol, 1.00 equiv) in ACN (15.0 mL) was stirred overnight at 60° C. The mixture was allowed to cool down to room temperature. The reaction was quenched with water at room temperature. The resulting mixture was extracted with CH₂Cl₂ (3× 100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford 5-[(1-hydroxy-5-nitro-2,3-dihydro-1H-inden-4-yl)oxy]-2-methyl-3H-isoindol-1-one (413 mg, 34.2%) as a light brown solid. LC-MS (ESI): m/z 341.15 [M+H]⁺

4-[(2-methyl-1-oxo-3H-isoindol-5-yl)oxy]-5-nitro-2,3-dihydro-1H-inden-1-yl di((2-bromoethyl)amino)phosphinate

To a stirred solution of 5-[(1-hydroxy-5-nitro-2,3-dihydro-1H-inden-4-yl)oxy]-2-methyl-3H-isoindol-1-one (395 mg, 1.16 mmol, 1.00 equiv) in THF (20.0 mL) was added LiHMDS (2.90 mL, 2.90 mmol, 2.50 equiv, 1.0 mol/L in THF) dropwise at −60° C. under nitrogen atmosphere. The resulting mixture was stirred for 20 min at −60° C. under nitrogen atmosphere. To the above mixture was added POCl₃ (445 mg, 2.90 mmol, 2.50 equiv) dropwise at −60° C. The resulting mixture was stirred for additional 30 min at −60° C. To the above mixture was added 2-bromoethan-1-amine hydrobromide (1.43 g, 6.97 mmol, 6.00 equiv) and DIEA (1.20 g, 9.29 mmol, 8.00 equiv) in portions at −60° C. The resulting mixture was stirred for additional 20 min at −60° C. and then stirred for 1 h at room temperature under nitrogen atmosphere. The reaction was quenched with Water at room temperature. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 4-[(2-methyl-1-oxo-3H-isoindol-5-yl)oxy]-5-nitro-2,3-dihydro-1H-inden-1-yl di((2-bromoethyl)amino)phosphinate (397 mg, 54.1%) as a yellow solid. LC-MS (ESI): m/z 631.05, 633.05, 635.05 [M+H]⁺

4-[(2-methyl-1-oxo-3H-isoindol-5-yl)oxy]-5-nitro-2,3-dihydro-1H-inden-1-yl bis(aziridin-1-yl)phosphinate

A mixture of 4-[(2-methyl-1-oxo-3H-isoindol-5-yl)oxy]-5-nitro-2,3-dihydro-1H-inden-1-yl di((2-bromoethyl)amino)phosphinate (377 mg, 0.60 mmol, 1.00 equiv), DIEA (154 mg, 1.19 mmol, 2.00 equiv) and Ag₂O (677 mg, 2.92 mmol, 4.90 equiv) in THF (128 mL) was stirred overnight at 70° C. under nitrogen atmosphere. The mixture was allowed to cool down to room temperature. The resulting mixture was filtered, the filter cake was washed with CH₂Cl₂ (3×50 mL). The filtrate was concentrated under reduced pressure. The resulting mixture was diluted with water (50 mL) and extracted with EtOAc (3×50 nmL). The combined organic layers were washed with brine (2×50 mL), dried over anhydrous Na₂SO₄. After filtration, the filtrate was concentrated under reduced pressure. The crude product was purified by Prep-HPLC with the following conditions: (Column: XBridge Prep C18 OBD Column, 50*250 mm, 10 μm; Mobile Phase A: water (0.05% NH₃H₂O), Mobile Phase B: ACN; Flow rate: 100 mL/min; Gradient: 10% B to 40% B in 30 min, 40% B; Wave Length: 254/220 nm) to afford 4-[(2-methyl-1-oxo-3H-isoindol-5-yl)oxy]-5-nitro-2,3-dihydro-1H-inden-1-yl bis(aziridin-1-yl)phosphinate (159 mg, 53.4%) as a light yellow oil. LC-MS (ESI): m/z 471.15 [M+H]⁺

(R)-4-((2-methyl-1-oxoisoindolin-5-yl)oxy)-5-nitro-2,3-dihydro-1H-inden-1-yl di(aziridin-1-yl)phosphinate & (S)-4-((2-methyl-1-oxoisoindolin-5-yl)oxy)-5-nitro-2,3-dihydro-1H-inden-1-yl di(aziridin-1-yl)phosphinate

4-[(2-methyl-1-oxo-3H-isoindol-5-yl)oxy]-5-nitro-2,3-dihydro-1H-inden-1-yl bis(aziridin-1-yl)phosphinate (159 mg, 0.337 mmol, 1.00 equiv) was separated by Prep-Chiral HPLC with the following conditions: Column: CHIRALPAK IC, 2*25 cm, 5 μm; Mobile Phase A: MtBE (10 mM NH₃-MeOH), Mobile Phase B: MeOH; Flow rate: 20 mL/min; Gradient: 50% B to 50% B in 13 min; Wave Length: 208/248 nm; RT1 (min): 7.98; RT2 (min): 10.4; Sample Solvent: MeOH: DCM=1:2; InJection Volume: 0.8 mL; Number Of Runs: 5.

Isomer 1 (42), 48.5 mg, 30.6%, ee>99%, LC-MS (ESI): m/z 471.15 [M+H]^{+ 1}H NMR (400 MHz, DMSO-d₆) δ 8.08 (d, J=8.2 Hz, 1H), 7.64 (d, J=8.3 Hz, 1H), 7.58 (dd, J=8.2, 0.8 Hz, 1H), 7.12 (d, J=2.2 Hz, 1H), 7.02 (dd, J=8.3, 2.4 Hz, 1H), 5.95 (q, J=6.6 Hz, 1H), 4.40 (s, 2H), 3.04 (s, 3H), 2.80-2.44 (m, 3H), 2.22-1.98 (m, 9H).

Isomer 2 (43), 49.0 mg, 30.9%, ee>99%, LC-MS (ESI): m/z 471.15 [M+H]^{+ 1}H NMR (400 MHz, DMSO-d₆) δ 8.08 (d, J=8.2 Hz, 1H), 7.64 (d, J=8.3 Hz, 1H), 7.58 (d, J=8.2 Hz, 1H), 7.12 (d, J=2.3 Hz, 1H), 7.02 (dd, J=8.3, 2.3 Hz, 1H), 5.95 (q, J=6.5 Hz, 1H), 4.40 (s, 2H), 3.04 (s, 3H), 2.79-2.43 (m, 3H), 2.28-1.98 (m, 9H).

### Illustration 12. Synthesis of (S)-4-(4-(1-methyl-1H-pyrazol-4-yl)phenoxy)-5-nitro-2,3-dihydro-1H-inden-1-yl di(aziridin-1-yl)phosphinate & (R)-4-(4-(1-methyl-1H-pyrazol-4-yl)phenoxy)-5-nitro-2,3-dihydro-1H-inden-1-yl di(aziridin-1-yl)phosphinate (58&59)

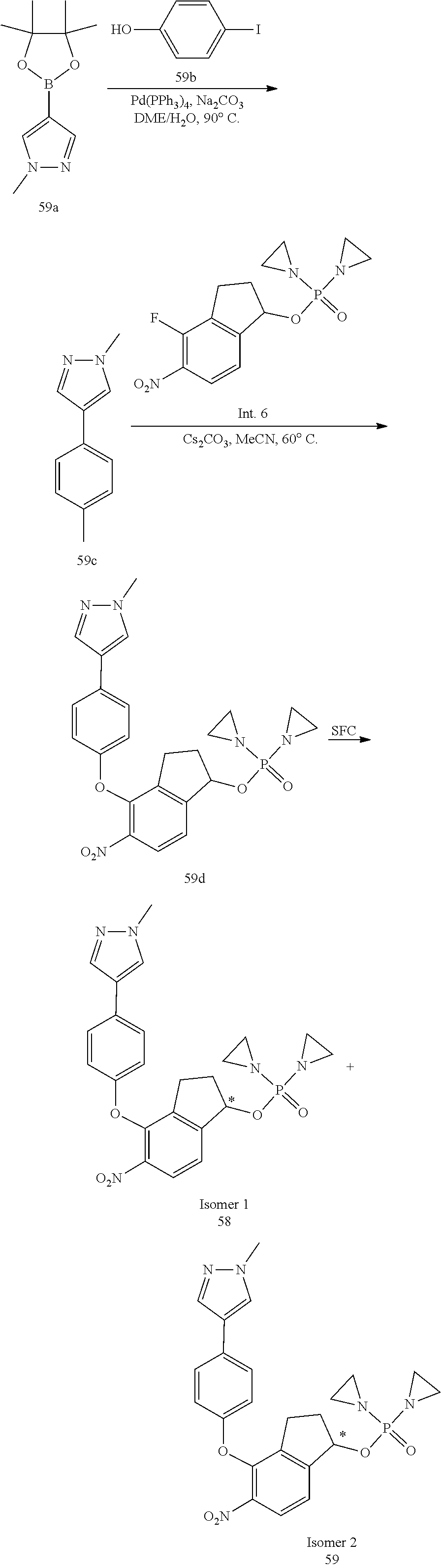

4-(1-methyl-1H-pyrazol-4-yl)phenol

To a mixture of 4-iodophenol (3.6 g, 16.3 mmol, 1.0 eq.), 1-methyl-4-(4,4,5,5-tetramethyl-1,3,2-dioxaborolan-2-yl)-1H-pyrazole (5.1 g, 24.5 mmol, 1.5 eq.) and Na₂CO₃ (5.2 g, 48.9 mmol, 3.0 eq.) in DME (100 mL) and H₂O (10 mL) was added Pd(PPh₃)₄ (1.88 g, 1.63 mmol, 0.1 eq.) at room temperature under N₂. The resulting solution was stirred at 90° C. for 16 hrs under N₂. After completion, the reaction mixture was cooled to room temperature, diluted with water (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford 4-(1-methyl-1H-pyrazol-4-yl)phenol (1.2 g, 6.90 mmol, 42%) as a yellow solid. LCMS (ESI): m/z 175 [M+H]⁺.

4-(4-(1-methyl-1H-pyrazol-4-yl)phenoxy)-5-nitro-2,3-dihydro-1H-inden-1-yl di(aziridin-1-yl)phosphinate

To a solution of 4-(1-methyl-1H-pyrazol-4-yl)phenol (73 mg, 0.42 mmol, 1.5 eq.) and 4-fluoro-5-nitro-2,3-dihydro-1H-inden-1-yl di(aziridin-1-yl)phosphinate (90 mg, 0.28 mmol, 1.0 eq.) in MeCN (5 mL) was added Cs₂CO₃ (273 mg, 0.84 mmol, 2.0 eq.). The reaction mixture was stirred at 60° C. for 1 hr. After completion, the reaction mixture was concentrated under reduced pressure. The residue was purified by Biotage® C18 column chromatography to give 4-(4-(1-methyl-1H-pyrazol-4-yl)phenoxy)-5-nitro-2,3-dihydro-1H-inden-1-yl di(aziridin-1-yl)phosphinate (85 mg, 0.18 mmol, 64%) as a gray oil in a stereo isomeric mixture form. ¹H NMR (400 MHz, CDCl₃) δ 7.88 (d, J=8.2 Hz, 1H), 7.69 (s, 1H), 7.54 (s, 1H), 7.50 (d, J=8.2 Hz, 1H), 7.40-7.36 (m, 2H), 6.90-6.84 (m, 2H), 6.06-5.94 (m, 1H), 3.94 (s, 3H), 2.94-2.83 (m, 1H), 2.67-2.50 (m, 2H), 2.33-2.17 (m, 9H). ³¹P NMR (162 MHz, CDCl₃) δ 29.99 (s). LC-MS (ESI): m/z 482.2 [M+H]⁺.

(S)-4-(4-(1-methyl-1H-pyrazol-4-yl)phenoxy)-5-nitro-2,3-dihydro-1H-inden-1-yl di(aziridin-1-yl)phosphinate & (R)-4-(4-(1-methyl-1H-pyrazol-4-yl)phenoxy)-5-nitro-2,3-dihydro-1H-inden-1-yl di(aziridin-1-yl)phosphinate

4-(4-(1-methyl-1H-pyrazol-4-yl)phenoxy)-5-nitro-2,3-dihydro-1H-inden-1-yl di(aziridin-1-yl)phosphinate (70 mg, 0.15 mmol) was further separated by Chiral SFC to give:

Isomer 1 (58), 21 mg, 30%; Retention time: 2.395 min, >99% ee. LC-MS (ESI): m/z 482.2 [M+H]⁺; ¹H NMR (400 MHz, CDCl₃) δ 7.88 (d, J=8.2 Hz, 1H), 7.69 (s, 1H), 7.55 (s, 1H), 7.50 (d, J=8.2 Hz, 1H), 7.41-7.35 (m, 2H), 6.90-6.84 (m, 2H), 6.05-5.95 (m, 1H), 3.93 (s, 3H), 2.93-2.82 (m, 1H), 2.66-2.50 (m, 2H), 2.29-2.18 (m, 9H). ³¹P NMR (162 MHz, CDCl₃) δ 29.97 (s).

Isomer 2 (59), 21 mg, 30%; Retention time: 3.070 min, >99% ee. LC-MS (ESI): m/z 482.2 [M+H]⁺; ¹H NMR (400 MHz, CDCl₃) δ 7.88 (d, J=8.2 Hz, 1H), 7.69 (s, 1H), 7.55 (s, 1H), 7.50 (d, J=8.2 Hz, 1H), 7.41-7.36 (m, 2H), 6.90-6.83 (m, 2H), 6.05-5.96 (m, 1H), 3.93 (s, 3H), 2.92-2.82 (m, 1H), 2.65-2.51 (m, 2H), 2.32-2.17 (m, 9H). ³¹P NMR (162 MHz, CDCl₃) δ 29.99 (s).

Analytical method: Column: ChiralPak IB, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

SFC Method: Instrument: Waters Thar 80 preparative SFC, Column: ChiralPak IB, 250×21.2 mm I.D., 5 μm, Mobile phase: A for CO₂ and B for MEOH+0.1% NH₃H₂O, Gradient: B 40%, Flow rate: 40 mL/min, Back pressure: 100 bar, Column temperature: 35° C., Wavelength: 254 nm, Cycle-time: 15 min, Eluted time: 2 H.

### Illustration 13. Synthesis of (S)-4-((3-methyl-2-oxo-2,3-dihydrobenzo[d]oxazol-5-yl)oxy)-5-nitro-2,3-dihydro-1H-inden-1-yl di(aziridin-1-yl)phosphinate & (S)-4-((3-methyl-2-oxo-2,3-dihydrobenzo[d]oxazol-5-yl)oxy)-5-nitro-2,3-dihydro-1H-inden-1-yl di(aziridin-1-yl)phosphinate (64&65)

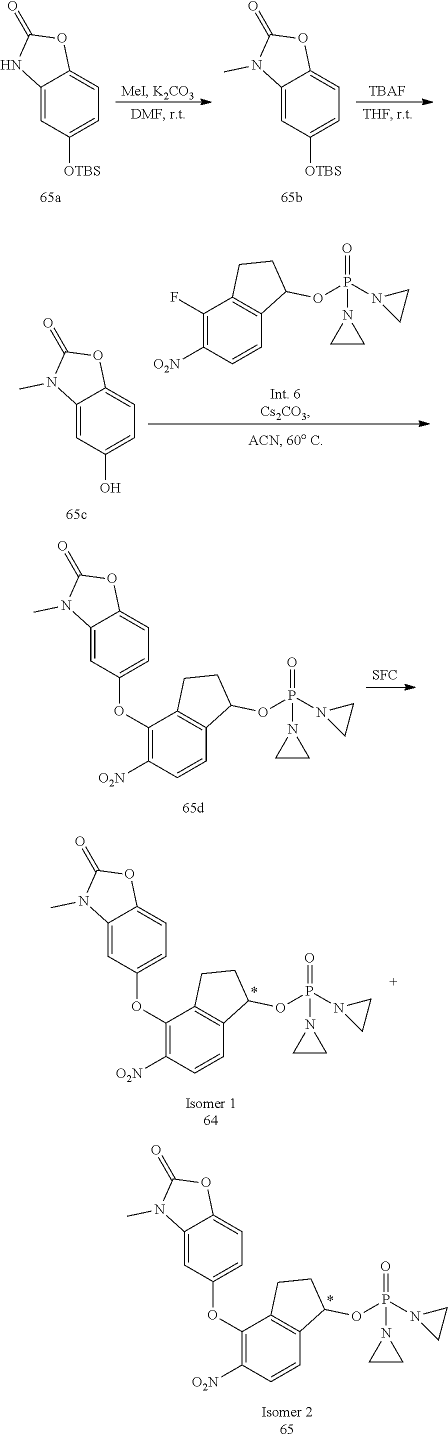

5-((tert-butyldimethylsilyl)oxy)-3-methylbenzo[d]oxazol-2 (3H)-one

To a stirred suspension of 5-((tert-butyldimethylsilyl)oxy)benzo[d]oxazol-2 (3/I)-one (2.2 g, 8.29 mmol, 1.0 eq.) and Cs₂CO₃ (5.4 g, 16.6 mmol, 2.0 eq.) in DMF (10 mL) was added CH₃I (0.77 mL, 12.4 mmol, 1.5 eq.) at room temperature. Then the mixture was stirred at room temperature for 2 hrs. After completion, the resulting mixture was poured into water (100 mL) and extracted with EtOAc (100 mL×3). The combined organic layers were washed with brine (50 mL), dried over anhydrous Na₂SO₄ and concentrated under reduced pressure. The residue was purified by flash column chromatography on silica gel to afford 5-((tert-butyldimethylsilyl)oxy)-3-methylbenzo[d]oxazol-2 (3H)-one (2 g, 7.16 mmol, 86%) as a yellow solid. LC-MS (ESI): m/z 280.2 [M+H]⁺.

5-hydroxy-3-methylbenzo[d]oxazol-2 (3H)-one

To a stirred suspension of 5-((tert-butyldimethylsilyl)oxy)-3-methylbenzo[d]oxazol-2 (3H)-one (2.0 g, 7.16 mmol, 1.0 eq.) in THF (25 mL) was added TBAF (11 mL) at room temperature. Then the mixture was stirred at room temperature for 2 hrs. After completion, the resulting mixture was poured into water (100 mL). and extracted with EtOAc (100 mL×3). The combined organic layers were washed with brine (50 mL), dried over anhydrous Na₂SO₄ and concentrated under reduced pressure. The residue was purified by flash column chromatography on silica gel to afford 5-hydroxy-3-methylbenzo[d]oxazol-2 (3H)-one (860 mg, 5.21 mmol, 73%) as a yellow solid. LCMS (ESI): m/z 166.1 [M+H]⁺.

4-((3-methyl-2-oxo-2,3-dihydrobenzo[d]oxazol-5-yl)oxy)-5-nitro-2,3-dihydro-1H-inden-1-yl di(aziridin-1-yl)phosphinate

To a solution of 5-hydroxy-3-methylbenzo[d]oxazol-2 (3H)-one (80 mg, 0.48 mmol, 1.5 eq.) and 4-fluoro-5-nitro-2,3-dihydro-1H-inden-1-yl di(aziridin-1-yl)phosphinate (105 mg, 0.32 mmol, 1.0 eq.) in MeCN (5 mL) was added Cs₂CO₃ (208 mg, 0.64 mmol, 2.0 eq.). The reaction mixture was stirred at 60° C. for 1 hr. After completion, the reaction mixture was concentrated under reduced pressure. The residue was purified by Biotage® C₁₈ column chromatography to give 4-((3-methyl-2-oxo-2,3-dihydrobenzo[d]oxazol-5-yl)oxy)-5-nitro-2,3-dihydro-1H-inden-1-yl di(aziridin-1-yl)phosphinate (40 mg, 85 μmol, 17%) as a yellow solid in a stereo isomeric mixture form. ¹H NMR (400 MHz, CDCl₃) δ 7.92-7.87 (m, 1H), 7.57-7.51 (m, 1H), 7.10-7.05 (m, 1H), 6.63-6.60 (m, 1H), 6.51 (dd, J=8.7, 2.5 Hz, 1H), 6.04-5.98 (m, 1H), 3.37 (s, 3H), 2.94-2.80 (m, 1H), 2.66-2.53 (m, 2H), 2.31-2.15 (m, 9H). ³¹P NMR (162 MHz, CDCl₃) δ 30.12 (s). LC-MS (ESI): m/z 473.1 [M+H]⁺.

(S)-4-((3-methyl-2-oxo-2,3-dihydrobenzo[d]oxazol-5-yl)oxy)-5-nitro-2,3-dihydro-1H-inden-1-yl di(aziridin-1-yl)phosphinate & (R)-4-((3-methyl-2-oxo-2,3-dihydrobenzo[d]oxazol-5-yl)oxy)-5-nitro-2,3-dihydro-1H-inden-1-yl di(aziridin-1-yl)phosphinate

4-((3-methyl-2-oxo-2,3-dihydrobenzo[d]oxazol-5-yl)oxy)-5-nitro-2,3-dihydro-1H-inden-1-yl di(aziridin-1-yl)phosphinate (40 mg, 85 μmol) was further separated by Chiral SFC to give:

Isomer 1 (64), 3.6 mg, 9%; Retention time: 4.259 min, >99% ee. LC-MS (ESI): m/z 473.1 [M+H]⁺; ¹H NMR (400 MHz, CDCl₃) δ 7.90 (d, J=8.2 Hz, 1H), 7.54 (d, J=8.3 Hz, 1H), 7.08 (d, J=8.7 Hz, 1H), 6.62 (t, J=3.4 Hz, 1H), 6.52 (dd, J=8.7, 2.5 Hz, 1H), 6.05-5.97 (m, 1H), 3.37 (s, 3H), 2.93-2.82 (m, 1H), 2.66-2.50 (m, 2H), 2.30-2.14 (m, 9H). ³¹P NMR (162 MHz, CDCl₃) δ 30.13 (s).

Isomer 2 (65), 4.0 mg, 9%; Retention time: 5.464 min, >99% ee. LC-MS (ESI): m/z 473.1 [M+H]⁺; ¹H NMR (400 MHz, CDCl₃) δ 7.93-7.87 (m, 1H), 7.56-7.49 (m, 1H), 7.11-7.04 (m, 1H), 6.66-6.59 (m, 1H), 6.55-6.48 (m, 1H), 6.04-5.98 (m, 1H), 3.40-3.32 (m, 3H), 2.93-2.82 (m, 1H), 2.66-2.51 (m, 2H), 2.33-2.14 (m, 9H). ³¹P NMR (162 MHz, CDCl₃) δ 30.13 (s).

Analytical method: Column: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

SFC Method: Instrument: SHIMADZU PREP SOLUTION SFC, Column: ChiralPak C-IG, 250×21.2 mm I.D., 5 μm, Mobile phase: A for CO₂ and B for MEOH+0.1% NH₃H₂O, Gradient: B 50%, Flow rate: 40 mL/min, Back pressure: 100 bar, Column temperature: 35° C., Wavelength: 220 nm, Cycle-time: 9 min, Eluted time: 2 h.

### Illustration 14. Synthesis of (S)-5-nitro-4-(3-(piperidine-1-carbonyl)phenoxy)-2,3-dihydro-1H-inden-1-yl di(aziridin-1-yl)phosphinate & (R)-5-nitro-4-(3-(piperidine-1-carbonyl)phenoxy)-2,3-dihydro-1H-inden-1-yl di(aziridin-1-yl)phosphinate (68&69)

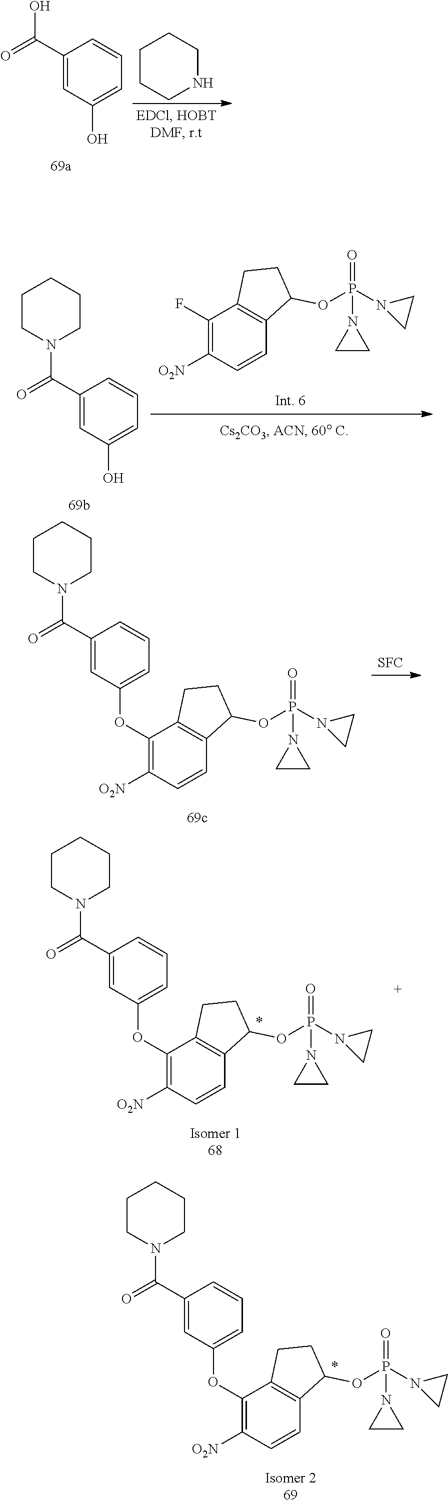

(3-hydroxyphenyl) (piperidin-1-yl) methanone

To a solution of 3-hydroxybenzoic acid (500 mg, 3.62 mmol, 1.0 eq.) and piperidine (462 mg, 5.43 mmol, 1.5 eq.) in DMF (10 mL) were added HOBt (734 mg, 5.43 mmol, 1.0 eq.) and EDCI (1.04 g, 5.43 mmol, 1.5 eq.). The mixture was stirred at room temperature for 2 hrs. After completion, the reaction mixture was diluted with H₂O (20 mL) and extracted with EtOAc (30 mL×3). The combined organic layers were dried over anhydrous Na₂SO₄ and concentrated under reduced pressure. The residue was triturated with EtOAc and filtered to afford (3-hydroxyphenyl) (piperidin-1-yl) methanone (500 mg, 2.44 mmol, 67%) as a white solid. LC-MS (ESI): m/z 206 [M+H]⁺.

5-nitro-4-(3-(piperidine-1-carbonyl)phenoxy)-2,3-dihydro-1H-inden-1-yl di(aziridin-1-yl)phosphinate

To a solution of (3-hydroxyphenyl) (piperidin-1-yl) methanone (100 mg, 0.49 mmol, 1.5 eq.) and 4-fluoro-5-nitro-2,3-dihydro-1H-inden-1-yl di(aziridin-1-yl)phosphinate (106 mg, 0.33 mmol, 1.0 eq.) in MeCN (5 mL) was added Cs₂CO₃ (215 mg, 0.66 mmol, 2.0 eq.). The reaction mixture was stirred at 60° C. for 1 hr. After completion, the reaction mixture was concentrated under reduced pressure. The residue was purified by Biotage® C₁₈ column chromatography to give 5-nitro-4-(3-(piperidine-1-carbonyl)phenoxy)-2,3-dihydro-1H-inden-1-yl di(aziridin-1-yl)phosphinate (86 mg, 17 μmol, 35%) as an oil in a stereo isomeric mixture form. ¹H NMR (400 MHz, CDCl₃) δ 7.89 (d, J=8.2 Hz, 1H), 7.53 (d, J=8.2 Hz, 1H), 7.34 (t, J=7.9 Hz, 1H), 7.09 (d, J=7.6 Hz, 1H), 6.93 (dd, J=8.2, 2.1 Hz, 1H), 6.84 (s, 1H), 6.04-5.96 (m, 1H), 3.74-3.58 (m, 2H), 3.38-3.24 (m, 2H), 2.93-2.83 (m, 1H), 2.68-2.51 (m, 2H), 2.32-2.08 (m, 9H), 1.73-1.64 (m, 4H), 1.53-1.43 (m, 2H). ³¹P NMR (162 MHz, CDCl₃) δ 29.88 (s). LC-MS (ESI): m/z 513.2 [M+H]⁺.

S)-5-nitro-4-(3-(piperidine-1-carbonyl)phenoxy)-2,3-dihydro-1H-inden-1-yl di(aziridin-1-yl)phosphinate & (R)-5-nitro-4-(3-(piperidine-1-carbonyl)phenoxy)-2,3-dihydro-1H-inden-1-yl di(aziridin-1-yl)phosphinate

5-nitro-4-(3-(piperidine-1-carbonyl)phenoxy)-2,3-dihydro-1H-inden-1-yl di(aziridin-1-yl)phosphinate (86 mg, 17 μmol) was further separated by Chiral SFC to give:

Isomer 1 (68), 25 mg, 29%; Retention time: 2.513 min, >99% ee. LC-MS (ESI): m/z 513.4 [M+H]⁺; ¹H NMR (400 MHz, CDCl₃) δ 7.87 (d, J=8.2 Hz, 1H), 7.51 (d, J=8.2 Hz, 1H), 7.32 (t, J=7.9 Hz, 1H), 7.10-7.05 (m, 1H), 6.95-6.88 (m, 1H), 6.84-6.80 (m, 1H), 6.02-5.95 (m, 1H), 3.72-3.57 (m, 2H), 3.35-3.20 (m, 2H), 2.91-2.79 (m, 1H), 2.65-2.48 (m, 2H), 2.26-2.16 (m, 9H), 1.70-1.56 (m, 4H), 1.53-1.41 (m, 2H). ³¹P NMR (162 MHz, CDCl₃) δ 29.86 (s).

Isomer 2 (69), 25 mg, 29%; Retention time: 2.746 min, >99% ee. LC-MS (ESI): m/z 513.4 [M+H]⁺; ¹H NMR (400 MHz, CDCl₃) δ 7.89 (d, J=8.2 Hz, 1H), 7.53 (d, J=8.2 Hz, 1H), 7.34 (t, J=7.9 Hz, 1H), 7.12-7.07 (m, 1H), 6.96-6.91 (m, 1H), 6.86-6.82 (m, 1H), 6.04-5.97 (m, 1H), 3.76-3.57 (m, 2H), 3.41-3.24 (m, 2H), 2.94-2.81 (m, 1H), 2.69-2.50 (m, 2H), 2.32-2.13 (m, 9H), 1.71-1.58 (m, 4H), 1.55-1.44 (m, 2H). ³¹P NMR (162 MHz, CDCl₃) δ 29.87 (s).

Analytical method: Column: ChiralPak IB, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

SFC Method: Instrument: SHIMADZU PREP SOLUTION SFC, Column: ChiralPak IB, 250×21.2 mm I.D., 5 μm, Mobile phase: A for CO₂ and B for MEOH+0.1% NH₃H₂O, Gradient: B 15%, Flow rate: 40 mL/min, Back pressure: 100 bar, Column temperature: 35° C., Wavelength: 220 nm, Cycle-time: 7 min, Eluted time: 3 h.

### Illustration 15. Synthesis of (S)-4-(3-(1-methyl-1H-pyrazol-4-yl)phenoxy)-5-nitro-2,3-dihydro-1H-inden-1-yl di(aziridin-1-yl)phosphinate & (R)-4-(3-(1-methyl-1H-pyrazol-4-yl)phenoxy)-5-nitro-2,3-dihydro-1H-inden-1-yl di(aziridin-1-yl)phosphinate (76&77)

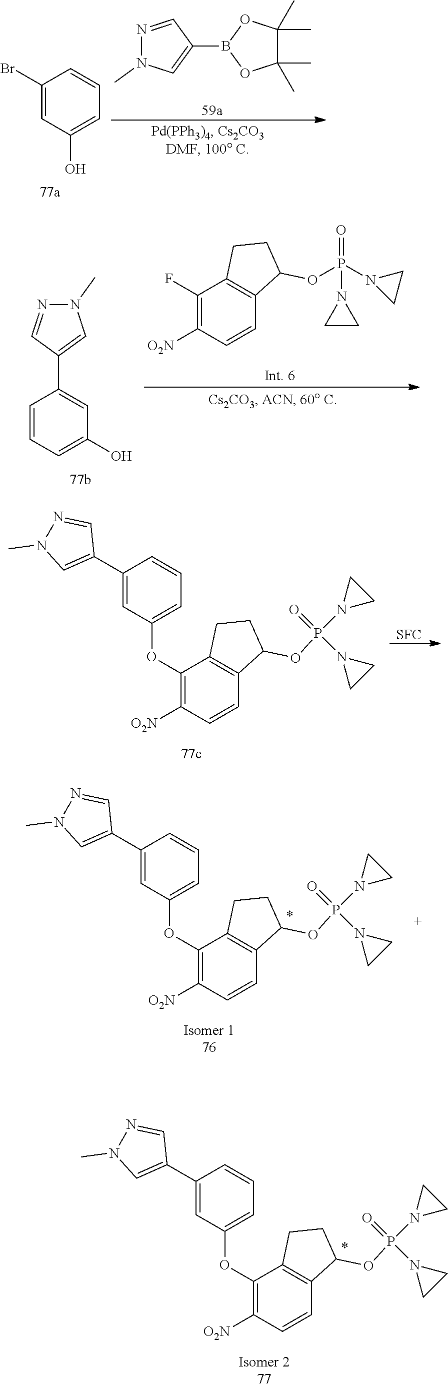

3-(1-methyl-1H-pyrazol-4-yl)phenol

To a mixture of 3-bromophenol (100 mg, 0.58 mmol, 1.0 eq.), 1-methyl-4-(4,4,5,5-tetramethyl-1,3,2-dioxaborolan-2-yl)-1H-pyrazole (144 mg, 0.69 mmol, 1.2 eq.) and Cs₂CO₃ (565 mg, 1.73 mmol, 3.0 eq.) in DMF (8 mL) was added Pd(PPh₃)₄ (33.4 mg, 29 μmol, 0.05 eq.) at room temperature under N₂. The resulting solution was stirred at 100° C. for 5 hrs under N₂. After completion, the reaction mixture was cooled to room temperature and filtered. The filtrate was diluted with water (25 mL) and extracted with EtOAc (25 mL×3). The organic layers were combined and washed with brine (25 mL), dried over anhydrous Na₂SO₄, and concentrated under reduced pressure. The residue was purified by flash column chromatography on silica gel to afford 3-(1-methyl-1H-pyrazol-4-yl)phenol (75 mg, 0.43 mmol, 74%) as a yellow solid. ¹H NMR (400 MHz, CDCl₃) δ 7.76 (s, 1H), 7.61 (s, 1H), 7.25-7.21 (m, 1H), 7.09-7.05 (m, 1H), 6.99-6.96 (m, 1H), 6.74-6.68 (m, 1H), 3.96 (s, 3H). 4-(3-(1-methyl-1H-pyrazol-4-yl)phenoxy)-5-nitro-2,3-dihydro-1H-inden-1-yl di(aziridin-1-yl)phosphinate

To a solution of 3-(1-methyl-1H-pyrazol-4-yl)phenol (40.2 mg, 0.23 mmol, 1.5 eq.) and 4-fluoro-5-nitro-2,3-dihydro-1H-inden-1-yl di(aziridin-1-yl)phosphinate (50 mg, 0.15 mmol, 1.0 eq.) in MeCN (5 mL) was added Cs₂CO₃ (100 mg, 0.31 mmol, 2.0 eq.). The reaction mixture was stirred at 60° C. for 1 hr. After completion, the reaction mixture was concentrated under reduced pressure. The residue was purified by Biotage® C18 column chromatography to give 4-(3-(1-methyl-1H-pyrazol-4-yl)phenoxy)-5-nitro-2,3-dihydro-1H-inden-1-yl di(aziridin-1-yl)phosphinate (47 mg, 98 μmol, 64%) as a gray oil in a stereo isomeric mixture form. ¹H NMR (400 MHz, CDCl₃) δ 7.89 (d, J=8.2 Hz, 1H), 7.70 (s, 1H), 7.57 (s, 1H), 7.51 (d, J=8.2 Hz, 1H), 7.30-7.15 (m, 2H), 7.05-7.00 (m, 1H), 6.65 (dd, J=8.1, 2.4 Hz, 1H), 6.04-5.97 (m, 1H), 3.93 (s, 3H), 2.94-2.84 (m, 1H), 2.68-2.49 (m, 2H), 2.34-2.16 (m, 9H). ³¹P NMR (162 MHz, CDCl₃) δ 29.97 (s). LC-MS (ESI): m/z482.2 [M+H]⁺.

(S)-4-(3-(1-methyl-1H-pyrazol-4-yl)phenoxy)-5-nitro-2,3-dihydro-1H-inden-1-yl di(aziridin-1-yl)phosphinate & (R)-4-(3-(1-methyl-1H-pyrazol-4-yl)phenoxy)-5-nitro-2,3-dihydro-1H-inden-1-yl di(aziridin-1-yl)phosphinate

4-(3-(1-methyl-1H-pyrazol-4-yl)phenoxy)-5-nitro-2,3-dihydro-1H-inden-1-yl di(aziridin-1-yl)phosphinate (55 mg, 0.11 mmol) was further separated by Chiral SFC to give:

Isomer 1 (76), 26.1 mg, 47%; Retention time: 4.999 min, >99% ee. LC-MS (ESI): m/z 482.1 [M+H]⁺; ¹H NMR (400 MHz, CDCl₃) δ 7.89 (d, J=8.2 Hz, 1H), 7.70 (s, 1H), 7.57 (s, 1H), 7.51 (d, J=8.2 Hz, 1H), 7.26-7.24 (m, 1H), 7.20-7.16 (m, 1H), 7.08-6.95 (m, 1H), 6.71-6.60 (m, 1H), 6.04-5.95 (m, 1H), 3.93 (s, 3H), 2.93-2.84 (m, 1H), 2.68-2.47 (m, 2H), 2.28-2.13 (m, 9H). ³¹P NMR (162 MHz, CDCl₃) δ 29.98 (s).

Isomer 2 (77), 26.8 mg, 49%; Retention time: 8.646 min, >99% ee. LC-MS (ESI): m/z 482.1 [M+H]⁺; ¹H NMR (400 MHz, CDCl₃) δ 7.89 (d, J=8.2 Hz, 1H), 7.70 (s, 1H), 7.57 (s, 1H), 7.51 (d, J=8.2 Hz, 1H), 7.29-7.26 (m, 1H), 7.19-7.16 (m, 1H), 7.04-7.01 (m, 1H), 6.68-6.62 (m, 1H), 6.04-5.96 (m, 1H), 3.93 (s, 3H), 2.94-2.84 (m, 1H), 2.67-2.50 (m, 2H), 2.31-2.15 (m, 9H). ³¹P NMR (162 MHz, CDCl₃) δ 29.98 (s).

Analytical method: Column: ChiralPak AD, 250×4.6 mm I.D., 5 μm, Mobile phase: A for CO₂ and B for methanol (0.05% DEA), Gradient: 8 min @B 50%, Flow rate: 1.8 mL/min, Back pressure: 100 bar, Column temperature: 35° C.

SFC Method: Instrument: SHIMADZU PREP SOLUTION SFC, Column: ChiralPak AD, 250×21.2 mm I.D., 5 μm, Mobile phase: A for CO₂ and B for MEOH+0.1% NH₃H₂O, Gradient: B 50%, Flow rate: 40 mL/min, Back pressure: 100 bar, Column temperature: 35° C., Wavelength: 220 nm, Cycle-time: 5.3 min, Eluted time: 2 h.

### Illustration 16 Synthesis of (R)-4-(3-(morpholine-4-carbonyl)phenoxy)-5-nitro-2,3-dihydro-1H-inden-1-yl di(aziridin-1-yl)phosphinate & (S)-4-(3-(morpholine-4-carbonyl)phenoxy)-5-nitro-2,3-dihydro-1H-inden-1-yl di(aziridin-1-yl)phosphinate (125&126)

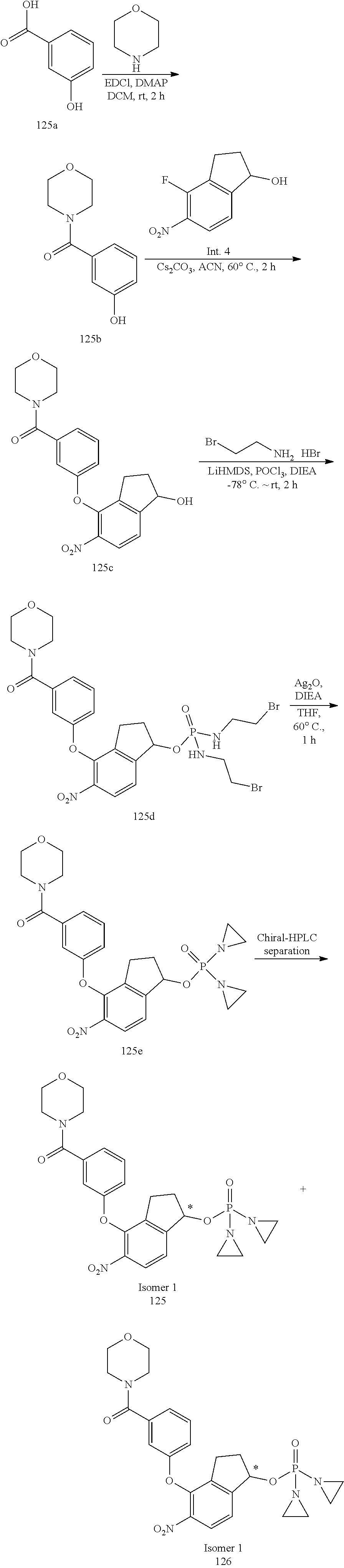

3-(morpholine-4-carbonyl)phenol

To a stirred solution of 3-hydroxybenzoic acid (10.0 g, 72.4 mmol, 1.00 equiv) and morpholine (6.94 g, 79.6 mmol, 1.10 equiv) in DCM (200 mL) at room temperature were added EDCI (15.3 g, 79.6 mmol, 1.10 equiv), DIEA (18.7 g, 145 mmol, 2.00 equiv) and DMAP (630 mg, 7.24 mmol, 0.10 equiv). The resulting mixture was stirred at room temperature for 2 h. The reaction was quenched with water. The resulting mixture was extracted with EtOAc (100 mL×3). The combined organic layers were dried over anhydrous Na₂SO₄. After filtration, the filtrate was concentrated under reduced pressure. The residue was purified by silica gel column chromatography, eluted with PE/EA (3:1) to afford 3-(morpholine-4-carbonyl)phenol as a light yellow oil (10.0 g, 66.7%). LC-MS (ESI): m/z 208.25 [M+H]⁺.

4-[3-(morpholine-4-carbonyl)phenoxy]-5-nitro-2,3-dihydro-1H-inden-1-ol

To a stirred solution of 4-fluoro-5-nitro-2,3-dihydro-1H-inden-1-ol (500 mg, 2.54 mmol, 1.00 equiv) in CH₃CN (10 mL) at room temperature were added 3-(morpholine-4-carbonyl)phenol (631 mg, 3.04 mmol, 1.20 equiv) and Cs₂CO₃ (1.65 g, 5.07 mmol, 2.00 equiv). The resulting mixture was stirred at 60° C. for 2 h. Then it was filtered through a short pad of celite. The pad was washed with EtOAc (3×10 mL). The combined filtrate was concentrated under reduced pressure. The residue was purified by silica gel column chromatography, eluted with PE/EA (1:2) to afford 4-[3-(morpholine-4-carbonyl)phenoxy]-5-nitro-2,3-dihydro-1H-inden-1-ol as a white solid (750 mg, 76.9%). LC-MS (ESI): m/z 385.20 [M+H]⁺

4-[3-(morpholine-4-carbonyl)phenoxy]-5-nitro-2,3-dihydro-1H-inden-1-yl di((2-bromoethyl)amino)phosphinate

To a stirred solution of 4-[3-(morpholine-4-carbonyl)phenoxy]-5-nitro-2,3-dihydro-1H-inden-1-ol (700 mg, 1.82 mmol, 1.00 equiv) in THF (125 mL) at −78° C. under nitrogen atmosphere was added LiHMDS (4.55 mL, 4.55 mmol, 2.5 equiv, 1.0 M in THF) dropwise. The resulting mixture was stirred at −78° C. for 30 min. To the above mixture was added POCl₃ (698 mg, 4.56 mmol, 2.50 equiv) dropwise and it was stirred at −78° C. for additional 30 min. To the above mixture at −78° C. was added 2-bromoethan-1-amine hydrobromide (2.24 g, 10.9 mmol, 6.00 equiv) and DIEA (1.88 g, 14.6 mmol, 8.00 equiv). The resulting mixture was stirred at −78° C. for 30 min. Then the mixture was warmed up to room temperature and stirred under nitrogen atmosphere for additional 2 h. The reaction was quenched with water and extracted with EtOAc (50 mL×3). The combined organic layers were dried over anhydrous Na₂SO₄. After filtration, the filtrate was concentrated under reduced pressure. The residue was purified by reversed-phase flash chromatography [with the following conditions: column, C18; mobile phase A: Water (0.1% NH₃·H₂O), mobile phase B: MeCN, 10% to 80% gradient in 30 min; detector, UV 254 nm] to afford 4-[3-(morpholine-4-carbonyl)phenoxy]-5-nitro-2,3-dihydro-1H-inden-1-yl di((2-bromoethyl)amino)phosphinate as a yellow solid (490 mg, 39.8%). LC-MS (ESI): m/z 674.80, 677.05, 679.05 [M+H]⁺

4-[3-(morpholine-4-carbonyl)phenoxy]-5-nitro-2,3-dihydro-1H-inden-1-yl bis(aziridin-1-yl)phosphinate

To a stirred solution of 4-[3-(morpholine-4-carbonyl)phenoxy]-5-nitro-2,3-dihydro-1H-inden-1-yl di((2-bromoethyl)amino)phosphinate (440 mg, 0.650 mmol, 1.00 equiv) in THF (10.0 mL) at room temperature were added Ag₂O (754 mg, 3.26 mmol, 5.00 equiv) and DIEA (420 mg, 3.26 mmol, 5.00 equiv). The resulting mixture was stirred at 60° C. for 1 h. The mixture was allowed to cool down to room temperature. The resulting mixture was filtered through a short pad of celite. The pad was washed with EtOAc (10 mL×3). The combined filtrate was diluted with water (50 mL) and extracted with EtOAc (20 mL×3). The combined organic layers were washed with brine (10 mL×3), dried over anhydrous Na₂SO₄. After filtration, the filtrate was concentrated under reduced pressure. The crude product was purified by Prep-HPLC [with the following conditions (Column: Ultimate XB-C18, 50*250 mm, 10 μm; Mobile Phase A: Water (0.05% NH₃·H₂O), Mobile Phase B: CH₃CN; Flow rate: 100 mL/min; Gradient: 25% B to 60% B in 930 min; Wave Length: 254/220 nm)] to afford 4-[3-(morpholine-4-carbonyl)phenoxy]-5-nitro-2,3-dihydro-1H-inden-1-yl bis(aziridin-1-yl)phosphinate as a yellow oil (120 mg, 35.9%). LC-MS (ESI): m/z 515.20 [M+H]⁺

(R)-4-(3-(morpholine-4-carbonyl)phenoxy)-5-nitro-2,3-dihydro-1H-inden-1-yl di(aziridin-1-yl)phosphinate & (S)-4-(3-(morpholine-4-carbonyl)phenoxy)-5-nitro-2,3-dihydro-1H-inden-1-yl di(aziridin-1-yl)phosphinate

The compound 4-[3-(morpholine-4-carbonyl)phenoxy]-5-nitro-2,3-dihydro-1H-inden-1-yl bis(aziridin-1-yl)phosphinate (110 mg, 0.21 mmol, 1.00 equiv) was separated by Prep-Chiral-HPLC [with the following conditions (Column: CHIRAL ART Cellulose-SB, 2*25 cm, 5 μm; Mobile Phase A: Hex: MtBE=1: 1 (0.5% 2M NH₃-MEOH), Mobile Phase B: MeOH; Flow rate: 20 mL/min; Gradient: 20% B to 20% B in 14 min; Wave Length: 222/230 nm; RT1 (min): 9.67; RT2 (min): 10.86; Sample Solvent: MeOH: DCM=1:1; Injection Volume: 0.4 mL; Number Of Runs: 21.

Isomer 1 (125), 30.0 mg, 27.3%, ee>97%. LC-MS (ESI): m/z 515.15 [M+H]⁺. ¹H NMR (400 MHz, Chloroform-d) § 7.89 (d, J=8.2 Hz, 1H), 7.54 (d, J=8.2 Hz, 1H), 7.36 (t, J=7.9 Hz, 1H), 7.11 (dt, J=7.5, 1.2 Hz, 1H), 6.95 (dd, J=8.2, 2.6 Hz, 1H), 6.87 (dd, J=2.7, 1.4 Hz, 1H), 6.06-5.94 (m, 1H), 4.01-3.24 (m, 8H), 2.98-2.78 (m, 1H), 2.77-2.50 (m, 2H), 2.41-2.13 (m, 9H). ³¹P NMR (162 MHz, Chloroform-d) δ 23.29.

Isomer 2 (126), 32.8 mg, 29.8%, ee>98%. LC-MS (ESI): m/z 515.20 [M+H]⁺. ¹H NMR (400 MHz, Chloroform-d) § 7.89 (d, J=8.2 Hz, 1H), 7.54 (d, J=8.2 Hz, 1H), 7.36 (t, J=7.9 Hz, 1H), 7.11 (dt, J=7.5, 1.2 Hz, 1H), 6.95 (ddd, J=8.3, 2.6, 1.0 Hz, 1H), 6.90-6.84 (m, 1H), 6.06-5.96 (m, 1H), 4.00-3.21 (m, 8H), 2.95-2.82 (m, 1H), 2.70-2.51 (m, 2H), 2.32-2.15 (m, 9H). ³¹P NMR (162 MHz, Chloroform-d) δ 23.29.

### Illustration 17. Synthesis of 7-nitro-8-phenoxy-3,4-dihydro-2H-1-benzopyran-4-yl bis(aziridin-1-yl)phosphinate (127) (General Procedure 6, 6,6 core 1-method A)

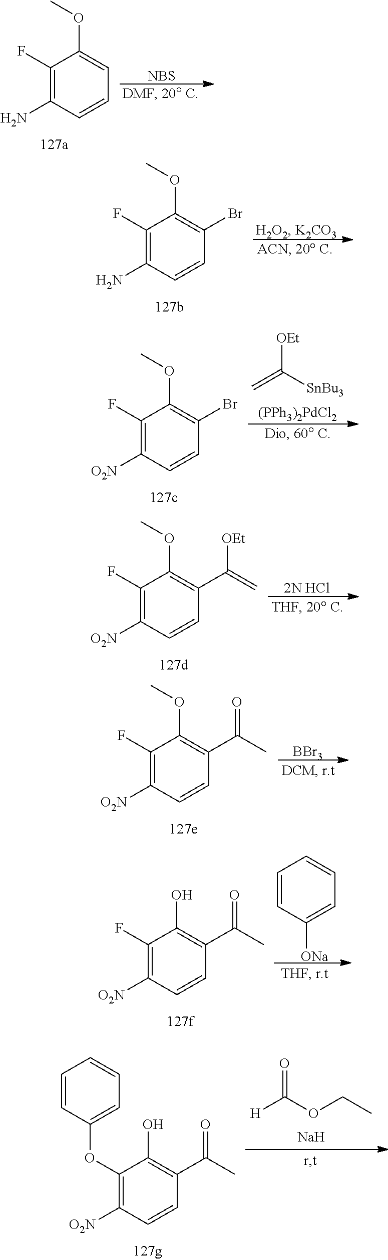

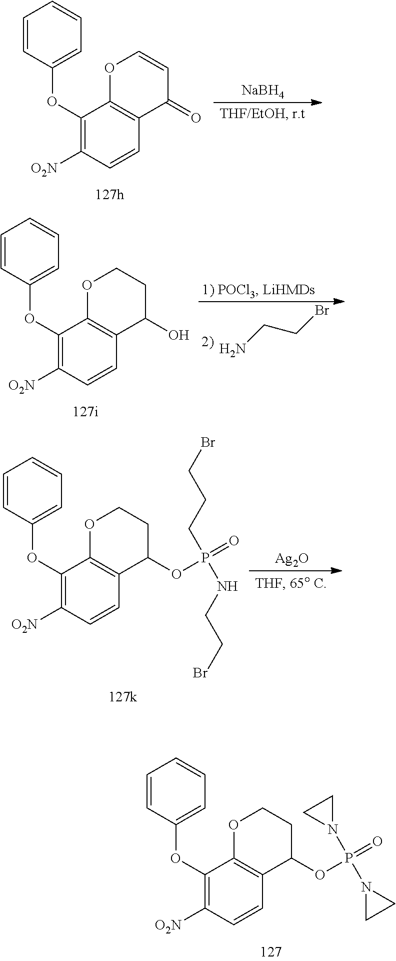

4-bromo-2-fluoro-3-methoxyaniline

To a solution of 2-fluoro-3-methoxyaniline (2.8 g, 19.83 mmol, 1.0 eq.) in DMF (50 mL) was added NBS (3.5 g, 19.84 mmol, 1.0 eq.) and the mixture was stirred at 20° C. for 4 hrs under nitrogen After completion, the mixture was poured into water (100 mL) and extracted with EtOAc (100 mL×3). The combined organic layers were dried over anhydrous Na₂SO₄, filtered and concentrated under reduced pressure to get 4-bromo-2-fluoro-3-methoxyaniline (4.3 g, 19.54 mmol, 99%) as a yellow oil. ¹H NMR (400 MHz, CDCl₃) δ 7.06 (dd, J=8.7, 2.1 Hz, 1H), 6.43-6.42 (m, 1H), 3.94 (s, 3H). 1-bromo-3-fluoro-2-methoxy-4-nitrobenzene

To a solution of 4-bromo-2-fluoro-3-methoxyaniline (3.2 g, 14.54 mmol, 1.0 eq.) in ACN (50 mL) were added K₂CO₃ (2.0 g, 14.54 mmol, 1.0 eq.) and H₂O₂ (49.5 mL, 1454.28 mmol, 30 wt %, 10.0 eq.). The mixture was stirred at 20° C. for 16 hrs under nitrogen. After completion, the mixture was quenched with saturated aqueous Na₂SO₃ solution (100 mL) and extracted with EtOAc (100 mL×3). The organic layers were dried over anhydrous Na₂SO₄, filtered and concentrated under reduced pressure. The residue was purified by flash column chromatography on silica gel to give 1-bromo-3-fluoro-2-methoxy-4-nitrobenzene (2.4 g, 9.60 mmol, 66%) as a yellow oil. ¹H NMR (400 MHz, CDCl₃) δ 7.68 (dd, J=9.0, 7.0 Hz, 1H), 7.48 (dd, J=9.0, 2.1 Hz, 1H), 4.04 (s, 3H).

1-(1-ethoxyethenyl)-3-fluoro-2-methoxy-4-nitrobenzene

To a solution of 1-bromo-3-fluoro-2-methoxy-4-nitrobenzene (2.4 g, 9.60 mmol, 1.0 eq.) in dioxane (60 mL) were added tributyl(1-ethoxyethenyl) stannane (3.5 g, 9.60 mmol, 1.0 eq.) and (PPh₃)₂PdCl₂ (0.37 g, 0.48 mmol, 0.05 eq.). The mixture was evaporated and backfilled with N₂ for three times. The mixture was stirred at 60° C. for 24 hrs under nitrogen atmosphere until the starting material was consumed completely. The reaction mixture was cooled to room temperature, quenched with KF solution (100 mL) and extracted with EtOAc (20 mL×3). The combined organic layers were dried over anhydrous Na₂SO₄, filtered and concentrated under reduced pressure. The residue was purified by flash column chromatography on silica gel to give 1-(1-ethoxyethenyl)-3-fluoro-2-methoxy-4-nitrobenzene (1.8 g, 7.46 mmol, 78%) as a yellow solid. ¹H NMR (400 MHz, DMSO-d₆) δ 7.87 (dd, J=8.9, 7.1 Hz, 1H), 7.44 (dd, J=8.9, 1.9 Hz, 1H), 4.75 (d, J=2.6 Hz, 1H), 4.62 (d, J=2.6 Hz, 1H), 3.93-3.88 (m, 2H), 3.87 (s, 3H), 1.32 (t, J=7.0 Hz, 3H).

1-(3-fluoro-2-methoxy-4-nitrophenyl) ethan-1-one

To a solution of 1-(1-ethoxyethenyl)-3-fluoro-2-methoxy-4-nitrobenzene (1.6 g, 6.63 mmol, 1.0 eq.) in THF (20 mL) was added HCl aqueous solution (20 mL, 2 N) and the mixture was stirred at 20° C. for 24 hrs under nitrogen atmosphere. After completion, the mixture was poured into water (20 mL) and extracted with EtOAc (10 mL×3). The combined organic layers were dried over anhydrous Na₂SO₄, filtered and concentrated under reduced pressure. The residue was purified by flash column chromatography on silica gel to give 1-(3-fluoro-2-methoxy-4-nitrophenyl) ethan-1-one (1.4 g, 6.57 mmol, 99%) as a yellow solid. ¹H NMR (400 MHz, CDCl₃) δ 7.73 (dd, J=8.7, 6.3 Hz, 1H), 7.51 (dd, J=8.7, 2.0 Hz, 1H), 4.12 (d, J=2.6 Hz, 3H), 2.64 (s, 3H).

1-(3-fluoro-2-hydroxy-4-nitrophenyl) ethan-1-one

To a solution of 1-(3-fluoro-2-methoxy-4-nitrophenyl) ethan-1-one (1.4 g, 6.57 mmol, 1.0 eq.) in DCM (20 mL) was added BBr₃ (4.9 g, 19.7 mmol, 3.0 eq.) and the mixture was stirred at room temperature for 24 hrs under nitrogen. After completion, the mixture was poured into water (20 mL) and extracted with DCM (10 mL×3). The combined organic layers were dried over anhydrous Na₂SO₄, filtered and concentrated under reduced pressure. The residue was purified by flash column chromatography on silica gel to give 1-(3-fluoro-2-hydroxy-4-nitrophenyl) ethan-1-one (400 mg, 2.01 mmol, 31%) as a yellow solid. ¹HNMR (400 MHz, CDCl₃) δ 12.47 (s, 1H), 7.66 (dd, J=8.9, 2.0 Hz, 1H), 7.48 (dd, J=8.9, 6.1 Hz, 1H), 2.72 (s, 3H).

1-(2-hydroxy-4-nitro-3-phenoxyphenyl) ethan-1-one

To a solution of 1-(3-fluoro-2-hydroxy-4-nitrophenyl) ethan-1-one (400 mg, 2.01 mmol, 1.0 eq.) in THF (20 mL) was added phenoxysodium (245 mg, 2.11 mmol, 1.05 eq.) at 0° C. and then warmed up at room temperature for 2 hrs until the starting material was consumed completely detected by TLC (PE:EA=4:1, Rf=0.8). The mixture was concentrated under reduced pressure. The residue was purified by flash column chromatography on silica gel to give 1-(2-hydroxy-4-nitro-3-phenoxyphenyl) ethan-1-one (180 mg, 0.66 mmol, 33%) as a yellow solid. ¹H NMR (400 MHz, CDCl₃) δ 12.59 (s, 1H), 7.75 (d, J=8.8 Hz, 1H), 7.39 (d, J=8.8 Hz, 1H), 7.34-7.27 (m, 2H), 7.08 (t, J=7.4 Hz, 1H), 6.92 (m, 2H), 2.72 (s, 3H).

7-nitro-8-phenoxy-4H-chromen-4-one

To a solution of 1-(2-hydroxy-4-nitro-3-phenoxyphenyl) ethan-1-one (180 mg, 0.66 mmol, 1.0 eq.) in ethyl formate (10 mL) was added NaH (158 mg, 3.95 mmol, 1.0 eq.) at 0° C. and then warmed up at room temperature for 2 hrs until the starting material was consumed completely detected by TLC (PE:EA=5:1, Rf=0.5). After completion, the reaction mixture was quenched with HCl aqueous solution (10 mL, 2 N) and extracted with DCM (10 mL×3). The combined organic phases were washed with water (10 mL), dried over anhydrous Na₂SO₄, filtered and concentrated under reduced pressure. The residue was purified by flash column chromatography on silica gel to give 7-nitro-8-phenoxy-4H-chromen-4-one (60 mg, 0.21 mmol, 32%) as a yellow solid. ¹H NMR (400 MHz, DMSO-d₆) δ 8.32 (d, J=6.0 Hz, 1H), 8.12 (s, 2H), 7.40-7.31 (m, 2H), 7.13 (t, J=7.4 Hz, 1H), 7.04-6.96 (m, 2H), 6.50 (d, J=6.0 Hz, 1H).

7-nitro-8-phenoxy-3,4-dihydro-2H-1-benzopyran-4-ol

To a solution of 7-nitro-8-phenoxy-4H-chromen-4-one (60 mg, 0.21 mmol, 1.0 eq.) in THF/EtOH (5 mL/5 mL) was added NaBH₄ (42 mg, 1.06 mmol, 5.0 eq.) and the resulting mixture was stirred at room temperature for 4 hrs under N₂ until the starting material was consumed completely. After completion, the reaction mixture was concentrated in vacuum to remove most of solvent. The residue was poured into water (20 mL) and extracted with EtOAc (10 mL×3). The combined organic phases were washed with water (20 mL), dried over anhydrous Na₂SO₄, filtered and concentrated under reduced pressure. The residue was purified by flash column chromatography on silica gel to give 7-nitro-8-phenoxy-3,4-dihydro-2H-1-benzopyran-4-ol (30 mg, 0.10 mmol, 49%) as a yellow solid. ¹H NMR (400 MHz, CDCl₃) δ 7.51 (d, J=8.5 Hz, 1H), 7.35 (d, J=8.6 Hz, 1H), 7.31-7.26 (m, 2H), 7.04 (t, J=7.4 Hz, 1H), 6.89-6.83 (m, 2H), 4.89 (t, J=4.6 Hz, 1H), 4.31-4.21 (m, 2H), 2.20-2.02 (m, 2H).

7-nitro-8-phenoxy-3,4-dihydro-2H-1-benzopyran-4-yl di((2-bromoethyl)amino)phosphinate

To a solution of 7-nitro-8-phenoxy-3,4-dihydro-2H-1-benzopyran-4-ol (30 mg, 0.10 mmol, 1.0 eq.) in THF (20 mL) was added LiHMDS (0.21 mL, 1N in THE solution, 2.0 eq.) at −40° C. under nitrogen atmosphere and the resulting mixture was stirred for 20 min. To the mixture was added POCl₃ (32 mg, 0.21 mmol, 2.0 eq.) at −40° C. and the reaction solution was stirred for another 20 min. Then, 2-bromoethan-1-amine hydrobromide (90 mg, 0.84 mmol, 8.4 eq.) and TEA (84.38 mg, 0.84 mmol, 8.4 eq.) were added into the mixture and stirred at −40° C. for 10 min. The reaction mixture was warmed to room temperature and stirred for 30 min, the reaction mixture was quenched with sat. NH₄Cl solution (10 mL) and extracted with DCM (10 mL×3). The combined organic phases were washed with water (10 mL), dried over anhydrous Na₂SO₄, filtered and concentrated under reduced pressure. The residue was purified by Biotage® C18 column chromatography to give 7-nitro-8-phenoxy-3,4-dihydro-2H-1-benzopyran-4-yl di((2-bromoethyl)amino)phosphinate (13 mg, 20 μmol, 21%) as a yellow oil. ¹H NMR (400 MHz, CDCl₃) δ 7.60-7.41 (m, 2H), 7.34-7.26 (m, 2H), 7.05 (t, J=6.9 Hz, 1H), 6.93-6.78 (m, 2H), 5.68-5.42 (m, 1H), 4.42-4.09 (m, 2H), 3.55-3.28 (m, 8H), 3.20-2.94 (m, 2H), 2.33-2.17 (m, 2H).

7-nitro-8-phenoxy-3,4-dihydro-2H-1-benzopyran-4-yl bis(aziridin-1-yl)phosphinate

To a solution of 7-nitro-8-phenoxy-3,4-dihydro-2H-1-benzopyran-4-yl di((2-bromoethyl)amino)phosphinate (13 mg, 20 μmol, 1.0 eq.) in THF (10 mL) was added silver (I) oxide (52 mg, 0.22 mmol, 11.0 eq.) and the mixture was stirred at 65° C. for 30 hrs under nitrogen atmosphere. After completion, the reaction mixture was filtered and the filtrate was concentrated under reduced pressure. The residue was purified by RP-prep HPLC to give 7-nitro-8-phenoxy-3,4-dihydro-2H-1-benzopyran-4-yl bis(aziridin-1-yl)phosphinate (8 mg, 19 μmol, 85%) as a yellow oil. ¹H NMR (400 MHz, CDCl₃) δ 7.55-7.46 (m, 2H), 7.32-7.27 (m, 2H), 7.05 (t, J=6.9 Hz, 1H), 6.95-6.81 (m, 2H), 5.75-5.60 (m, 1H), 4.38-4.19 (m, 2H), 2.36-2.17 (m, 10H). ³¹P NMR (162 MHz, CDCl₃) δ 29.99 (s). LCMS (ESI): m/z 440.0 [M+Na]⁺.

### Illustration 18. Synthesis of (R)-8-(4-(dimethylcarbamoyl)phenoxy)-7-nitrochroman-4-yl di(aziridin-1-yl)phosphinate & (S)-8-(4-(dimethylcarbamoyl)phenoxy)-7-nitrochroman-4-yl di(aziridin-1-yl)phosphinate (130&131) (General Procedure6, 6,6 core 1-method B)

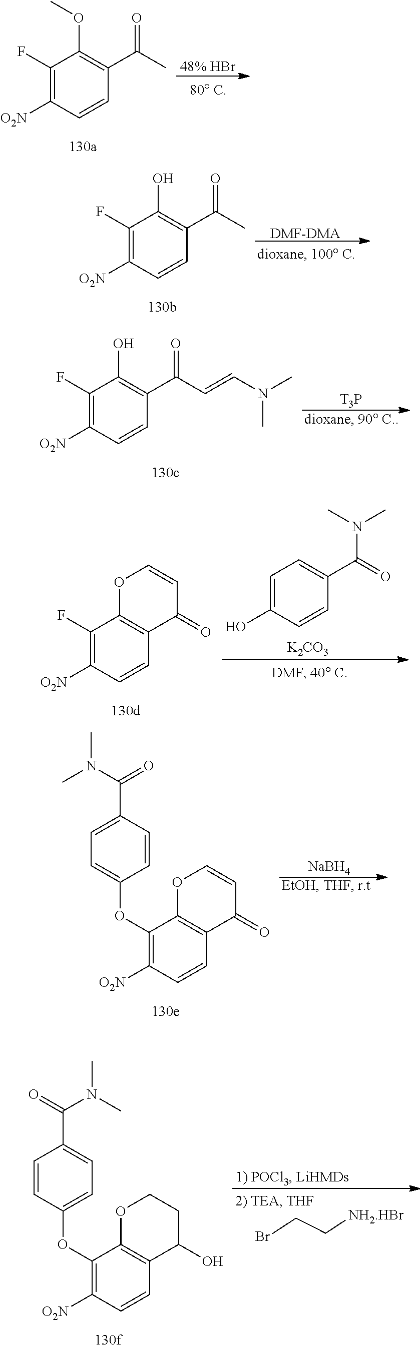

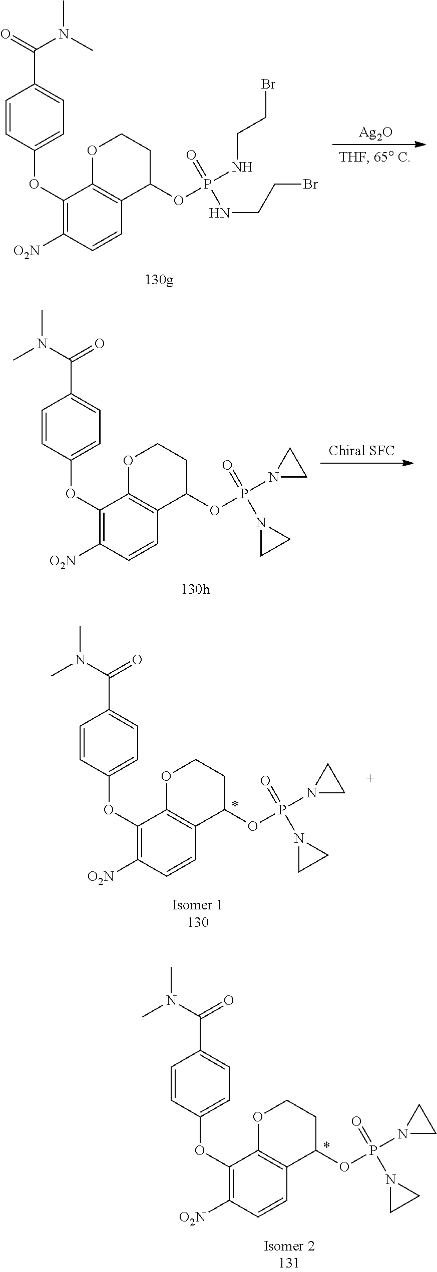

1-(3-fluoro-2-hydroxy-4-nitrophenyl) ethan-1-one

A solution of 1-(3-fluoro-2-methoxy-4-nitrophenyl) ethan-1-one (5.8 g, 27.2 mmol, 1.0 eq.) in HBr (50 mL, 48 wt %) was stirred at 80° C. overnight. After completion, the reaction mixture was concentrated under reduced pressure. The residue was purified by flash column chromatography on silica gel to afford 1-(3-fluoro-2-hydroxy-4-nitrophenyl) ethan-1-one (5 g, 25.1 mmol, 92%) as a yellow oil. LC-MS (ESI): m/z 200 [M+H]⁺.

3-(dimethylamino)-1-(3-fluoro-2-hydroxy-4-nitrophenyl) prop-2-en-1-one

To a solution of 1-(3-fluoro-2-hydroxy-4-nitrophenyl) ethan-1-one (1 g, 5.02 mmol, 1.0 eq.) in dry dioxane (10 mL) was added DMF-DMA (0.81 mL, 6.03 mmol, 1.2 eq.). The reaction mixture was stirred at 100° C. for 10 min by microwave. After completion, the reaction mixture was used directly for the next step without further purification. LC-MS (ESI): m/z 255 [M+H]⁺.

8-fluoro-7-nitro-4H-chromen-4-one

T₃P (3.00 g, 4.72 mmol, 50 wt % in EtOAc) was added into the above mixture. The reaction was stirred at 90° C. for 10 min by microwave. After completion, the mixture was diluted with H₂O (10 mL) and extracted with EtOAc (6 mL×3). The organic layers were combined and washed with brine (15 mL), dried over anhydrous Na₂SO₄, and concentrated under reduced pressure. The residue was purified by flash column chromatography on silica gel to afford 8-fluoro-7-nitro-4H-chromen-4-one (400 mg, 1.91 mmol, 41%) as a brown solid. LC-MS (ESI): m/z 210 [M+H]⁺.

N,N-dimethyl-4-((7-nitro-4-oxo-4H-chromen-8-yl)oxy)benzamide

To a solution of 8-fluoro-7-nitro-4H-chromen-4-one (120 mg, 0.57 mmol, 1.0 eq.) in DMF (3 mL) were added 4-hydroxy-N,N-dimethylbenzamide (95 mg, 0.57 mmol, 1.0 eq.) and K₂CO₃ (159 mg, 1.15 mmol, 2.0 eq.). The reaction mixture was stirred 40° C. for 1 hr. After completion, the mixture was diluted with H₂O (10 mL) and extracted with EtOAc (6 mL×3). The organic layers were combined and washed with brine (15 mL), dried over anhydrous Na₂SO₄ and concentrated under reduced pressure. The residue was purified by flash column chromatography on silica gel to afford N,N-dimethyl-4-((7-nitro-4-oxo-4H-chromen-8-yl)oxy)benzamide (160 mg, 0.45 mmol, 79%) as a yellow solid. LC-MS (ESI): m/z 355 [M+H]⁺.

4-((4-hydroxy-7-nitrochroman-8-yl)oxy)-N,N-dimethylbenzamide

To a solution of N,N-dimethyl-4-((7-nitro-4-oxo-4H-chromen-8-yl)oxy)benzamide (170 mg, 0.48 mmol, 1.0 eq.) in a mixture of EtOH (1.5 mL) and THF (1.5 mL) was added NaBH₄ (91.2 mg, 2.40 mmol, 5.0 eq.). The reaction mixture was stirred at room temperature for 1 hr. After completion, the mixture was quenched with NaHCO₃ (10 mL, aq. sat.) and extracted with EtOAc (6 mL×3). The organic layers were combined and washed with brine (15 mL), dried over anhydrous Na₂SO₄ and concentrated under reduced pressure. The residue was purified by flash column chromatography on silica gel to afford 4-((4-hydroxy-7-nitrochroman-8-yl)oxy)-N,N-dimethylbenzamide (120 mg, 0.34 mmol, 70%) as a yellow solid. LC-MS (ESI): m/z 359 [M+H]⁺.

8-(4-(dimethylcarbamoyl)phenoxy)-7-nitrochroman-4-yl di((2-bromoethyl)amino)phosphinate

To a solution of 4-((4-hydroxy-7-nitrochroman-8-yl)oxy)-N,N-dimethylbenzamide (80 mg, 0.22 mmol, 1.0 eq.) in THF (20 mL) was added LiHMDS (0.44 mL, 1 M in THF, 0.44 mmol, 2.0 eq.) dropwise at −60° C. under N₂ and the resulting solution was stirred at −60° C. for 20 min under N₂. POCl₃ (0.042 mL, 0.44 mmol, 2.0 eq.) in THF (0.5 mL) was quickly added and the resulting mixture was stirred at −60° C. for 15 min. 2-Bromoethylamine hydrobromide (320 mg, 1.56 mmol, 7.0 eq.) and TEA (0.37 mL, 2.68 mmol, 12.0 eq.) were added and the mixture was stirred at −60° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 8-(4-(dimethylcarbamoyl)phenoxy)-7-nitrochroman-4-yl di((2-bromoethyl)amino)phosphinate (60 mg, 92 μmol, 41%) as a yellow oil. LC-MS (ESI): m/z 690.2 [M+H+MeCN]⁺.

8-(4-(dimethylcarbamoyl)phenoxy)-7-nitrochroman-4-yl di(aziridin-1-yl)phosphinate

To a solution of 8-(4-(dimethylcarbamoyl)phenoxy)-7-nitrochroman-4-yl di((2-bromoethyl)amino)phosphinate (60 mg, 92 μmol, 1.0 eq.) in THF (10 mL) were added Ag₂O (214 mg, 0.92 mmol, 10.0 eq.) and DIEA (0.152 mL, 0.92 mmol, 10.0 eq.). The reaction solution was stirred at 70° C. for 18 hrs under N₂. After completion, the reaction mixture was filtered and the filtrate was concentrated under reduced pressure. The residue was purified by Biotage® C18 column chromatography to give 8-(4-(dimethylcarbamoyl)phenoxy)-7-nitrochroman-4-yl di(aziridin-1-yl)phosphinate (35 mg, 72 μmol, 78%) as a white solid in a stereo isomeric mixture form. ¹H NMR (400 MHz, CDCl₃) δ 77.51 (s, 2H), 7.43-7.33 (m, 2H), 6.93-6.81 (m, 2H), 5.78-5.55 (m, 1H), 4.40-4.13 (m, 2H), 3.08 (s, 3H), 3.04 (s, 3H), 2.41-2.06 (m, 10H). ³¹P NMR (162 MHz, CDCl₃) δ 30.01 (s). LC-MS (ESI): m/z 489.2 [M+H]⁺.

(R)-8-(4-(dimethylcarbamoyl)phenoxy)-7-nitrochroman-4-yl di(aziridin-1-yl)phosphinate & (S)-8-(4-(dimethylcarbamoyl)phenoxy)-7-nitrochroman-4-yl di(aziridin-1-yl)phosphinate,

8-(4-(dimethylcarbamoyl)phenoxy)-7-nitrochroman-4-yl di(aziridin-1-yl)phosphinate (35 mg, 72 μmol, 1.0 eq.) was further separated by Chiral SFC to give:

Isomer 1 (130), 5 mg, 14%, Retention time: 3.008 min, >99% ee. LC-MS (ESI): m/z 489.2 [M+H]⁺; ¹H NMR (400 MHz, CDCl₃) δ 7.51 (s, 2H), 7.38 (d, J=8.6 Hz, 2H), 6.87 (d, J=8.6 Hz, 2H), 5.73-5.59 (m, 1H), 4.41-4.14 (m, 2H), 3.08 (s, 3H), 3.04 (s, 3H), 2.39-2.08 (m, 10H). ³¹P NMR (162 MHz, CDCl₃) δ 30.02 (s).

Isomer 2 (131), 6 mg, 17%, Retention time: 4.757 min, 99.4% ee. LC-MS (ESI): m/z 489.2 [M+H]⁺; ¹H NMR (400 MHz, CDCl₃) δ 7.52 (d, J=8.0 Hz, 2H), 7.42-7.34 (m, 2H), 6.93-6.80 (m, 2H), 5.73-5.58 (m, 1H), 4.38-4.14 (m, 2H), 3.08 (s, 3H), 3.04 (s, 3H), 2.34-2.15 (m, 10H). ³¹P NMR (162 MHz, CDCl₃) δ 30.02 (s).

Analytical method: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C.

SFC Method: Instrument: Waters Thar 80 preparative SFC, Column: ChiralPak AD, 250×21.2 mm I.D., 5 μm, Mobile phase: A for CO₂ and B for MEOH+0.1% NH₃H₂O, Gradient: B 40%, Flow rate: 40 mL/min, Back pressure: 100 bar, Column temperature: 35° C., Wavelength: 220 nm, Cycle-time: 10 min, Eluted time: 1.5 H.

### Illustration 19. Synthesis of (R)-8-((2-cyclopropyl-3-oxoisoindolin-5-yl)oxy)-7-nitrochroman-4-yl di(aziridin-1-yl)phosphinate & (S)-8-((2-cyclopropyl-3-oxoisoindolin-5-yl)oxy)-7-nitrochroman-4-yl di(aziridin-1-yl)phosphinate

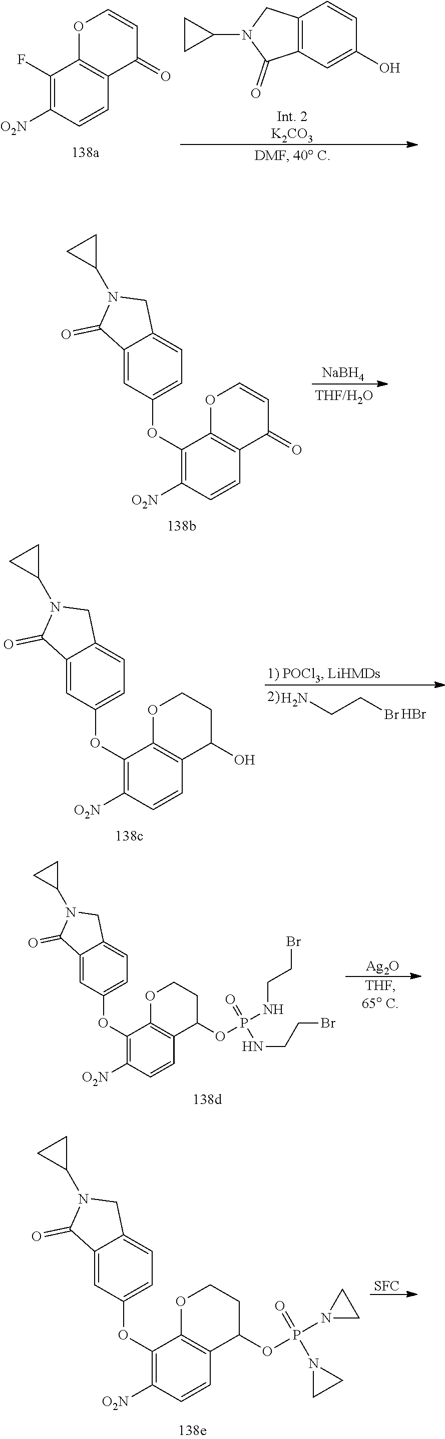

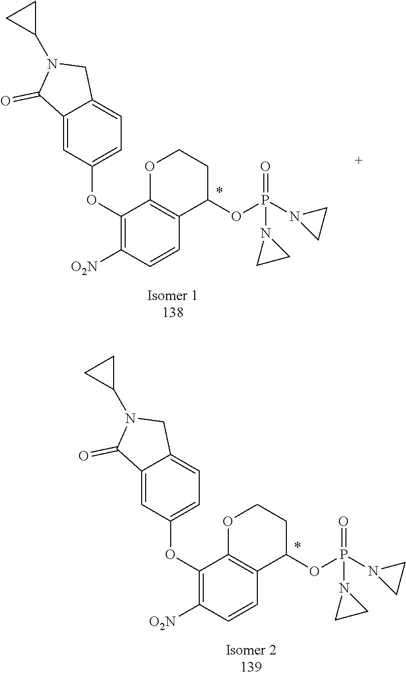 [SMILES: O=C1c2cc(Oc3c([N+](=O)[O-])ccc4c3OCCC4OP(=O)(N3CC3)N3CC3)ccc2CN1C1CC1 ; O=C1c2cc(Oc3c([N+](=O)[O-])ccc4c3OCCC4OP(=O)(N3CC3)N3CC3)ccc2CN1C1CC1]

2-cyclopropyl-6-((7-nitro-4-oxo-4H-chromen-8-yl)oxy) isoindolin-1-one

To a solution of 8-fluoro-7-nitro-4H-chromen-4-one (200 mg, 0.96 mmol, 1.0 eq.) and 2-cyclopropyl-6-hydroxyisoindolin-1-one (271 mg, 1.43 mmol, 1.5 eq.) in DMF (3 mL) was added K₂CO₃ (264 mg, 1.91 mmol, 2.0 eq) and the reaction mixture was stirred at 40° C. for 1 hr under nitrogen atmosphere. After completion, the reaction mixture was concentrated under reduced pressure. The residue was purified by Biotage® C18 column chromatography to give 2-cyclopropyl-6-((7-nitro-4-oxo-4H-chromen-8-yl)oxy) isoindolin-1-one (280 mg, 0.74 mmol, 77%) as a yellow oil. LC-MS (ESI): m/z 379 [M+H]⁺.

2-cyclopropyl-6-((4-hydroxy-7-nitrochroman-8-yl)oxy) isoindolin-1-one

To a solution of 2-cyclopropyl-6-((7-nitro-4-oxo-4H-chromen-8-yl)oxy) isoindolin-1-one (230 mg, 0.61 mmol, 1.0 eq.) in THF/H₂O (40 mL/4 mL) was added NaBH₄ (62 mg, 1.82 mmol, 3.0 eq.) at 0° C. The reaction mixture was warmed up to 25° C. and stirred for 1 hr. After completion, the reaction mixture was quenched by adding NH₄Cl (2 mL, aq. sat.) and extracted with EtOAc (10 mL×2). The organic layers were combined and washed with brine (10 mL), dried over anhydrous Na₂SO₄ and concentrated under reduced pressure. The residue was purified by flash column chromatography on silica gel to give 2-cyclopropyl-6-((4-hydroxy-7-nitrochroman-8-yl)oxy) isoindolin-1-one (200 mg, 0.52 mmol, 86%) as a yellow solid. LC-MS (ESI): m/z 383 [M+H]⁺.

8-((2-cyclopropyl-3-oxoisoindolin-5-yl)oxy)-7-nitrochroman-4-yl di((2-bromoethyl)amino)phosphinate

To a solution of 2-cyclopropyl-6-((4-hydroxy-7-nitrochroman-8-yl)oxy)isoindolin-1-one (220 mg, 0.58 mmol, 1.0 eq.) in THF (6 mL) was added LiHMDS (1.2 mL, 1.16 mmol, 2.0 eq.) at −60° C. The mixture was stirred at −60° C. for 15 min. POCl₃ (176 mg, 1.15 mmol, 2.0 eq.) was added and the reaction mixture was stirred at −60° C. for 15 min. 2-bromoethan-1-amine hydrobromide (707 mg, 3.45 mmol, 6.0 eq.) and TEA (699 mg, 6.90 mmol, 12.0 eq.) were added into the mixture. Then the mixture was stirred at −60° C. for 30 min. After completion, the reaction mixture was quenched with saturated NH₄Cl aqueous solution (10 mL) and extracted with EtOAc (10 mL). The organic layers was combined and washed brine (10 mL), dried over anhydrous Na₂SO₄ and concentrated under reduced pressure. The residue was purified by Biotage® C18 column chromatography to give 8-((2-cyclopropyl-3-oxoisoindolin-5-yl)oxy)-7-nitrochroman-4-yl di((2-bromoethyl)amino)phosphinate (160 mg, 0.24 mmol, 41%) as a yellow oil. LC-MS (ESI): m/z 673 [M+H]⁺.

8-((2-cyclopropyl-3-oxoisoindolin-5-yl)oxy)-7-nitrochroman-4-yl di(aziridin-1-yl)phosphinate

To a solution of 8-((2-cyclopropyl-3-oxoisoindolin-5-yl)oxy)-7-nitrochroman-4-yl di((2-bromoethyl)amino)phosphinate (160 mg, 0.24 mmol, 1.0 eq.) in THF (5 mL) were added DIEA (307 mg, 2.37 mmol, 10.0 eq.) and Ag₂O (548 mg, 2.37 mmol, 10.0 eq.). The reaction mixture was stirred at 70° C. for 12 hrs under nitrogen atmosphere. After completion, the reaction mixture was cooled to 25° C. and filtered. The filter cake was washed with DCM (10 mL). The combined filtrates was concentrated under reduced pressure. The residue was purified by Biotage® C18 column chromatography to give 8-((2-cyclopropyl-3-oxoisoindolin-5-yl)oxy)-7-nitrochroman-4-yl di(aziridin-1-yl)phosphinate (50 mg, 98 mmol, 41%) as a yellow oil in a stereo isomeric mixture form.

(R)-8-((2-cyclopropyl-3-oxoisoindolin-5-yl)oxy)-7-nitrochroman-4-yl di(aziridin-1-yl)phosphinate & (S)-8-((2-cyclopropyl-3-oxoisoindolin-5-yl)oxy)-7-nitrochroman-4-yl di(aziridin-1-yl)phosphinate

8-((2-cyclopropyl-3-oxoisoindolin-5-yl)oxy)-7-nitrochroman-4-yl di(aziridin-1-yl)phosphinate (10 mg, 20 μmol) was further separated by Chiral SFC to give:

Isomer 1 (138), 2.0 mg, 20%, Retention time: 4.724 min, >99% ee. LC-MS (ESI): m/z 513.1 [M+H]⁺; ¹H NMR (400 MHz, CDCl₃) δ 7.52 (s, 2H), 7.38-7.33 (m, 1H), 7.26-7.22 (m, 1H), 7.14-7.06 (m, 1H), 5.70-5.62 (m, 1H), 4.28 (s, 3H), 4.26-4.21 (m, 1H), 2.98-2.87 (m, 1H), 2.29-2.18 (m, 10H), 0.92-0.85 (m, 4H). ³¹P NMR (162 MHz, CDCl₃) δ 29.74 (s).

Isomer 2 (139), 2.0 mg, 20%, Retention time: 5.854 min, >99% ee. LC-MS (ESI): m/z 513.1 [M+H]⁺; ¹H NMR (400 MHz, CDCl₃) δ 7.54-7.50 (m, 2H), 7.39-7.34 (m, 1H), 7.26-7.23 (m, 1H), 7.11-7.04 (m, 1H), 5.71-5.59 (m, 1H), 4.28 (s, 3H), 4.25-4.20 (m, 1H), 2.96-2.88 (m, 1H), 2.29-2.19 (m, 10H), 0.97-0.85 (m, 4H). ³¹P NMR (162 MHz, CDCl₃) δ 29.74 (s).

Analytical method: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C.

SFC Method: Instrument: Waters Thar 80 preparative SFC, Column: ChiralPak AD, 250×21.2 mm I.D., 5 μm, Mobile phase: A for CO₂ and B for MEOH+0.1% NH₃H₂O, Gradient: B 32%, Flow rate: 40 mL/min, Back pressure: 100 bar, Column temperature: 35° C., Wavelength: 220 nm, Cycle-time: 15 min, Eluted time: 3 H.

### Illustration 20. Synthesis of ((R)-6-(4-(dimethylcarbamoyl)phenoxy)-5-nitro-2,3-dihydro-1H-inden-1-yl di(aziridin-1-yl)phosphinate & ((S)-6-(4-(dimethylcarbamoyl)phenoxy)-5-nitro-2,3-dihydro-1H-inden-1-yl di(aziridin-1-yl)phosphinate (140&141) (General Procedure 9, 6,5 core 4-method A)

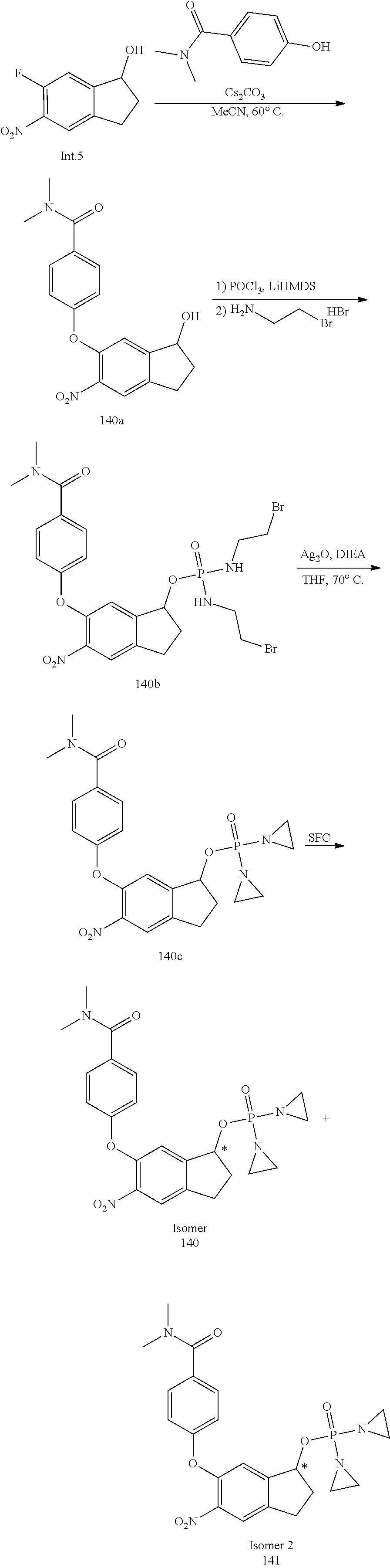

4-((3-hydroxy-6-nitro-2,3-dihydro-1H-inden-5-yl)oxy)-N,N-dimethylbenzamide

To a solution of 6-fluoro-5-nitro-2,3-dihydro-1H-inden-1-ole (140 mg, 0.71 mmol, 1.0 eq.) and 4-hydroxy-N,N-dimethylbenzamide (176 mg, 1.07 mmol, 1.5 eq.) in ACN (10 mL) was added Cs₂CO₃ (462 mg, 1.42 mmol, 2.0 eq.). The reaction mixture was stirred at 60° C. for 2 hrs. After completion, the reaction mixture was concentrated under reduced pressure. The residue was purified by Biotage® C18 column chromatography to give 4-((3-hydroxy-6-nitro-2,3-dihydro-1H-inden-5-yl)oxy)-N,N-dimethylbenzamide (135 mg, 0.39 mmol, 56%) as a white solid. LC-MS (ESI): m/z 343 [M+H]⁺.

6-(4-(dimethylcarbamoyl)phenoxy)-5-nitro-2,3-dihydro-1H-inden-1-yl di((2-bromoethyl)amino)phosphinate

To a solution of 4-((3-hydroxy-6-nitro-2,3-dihydro-1H-inden-5-yl)oxy)-N,N-dimethylbenzamide (130 mg, 0.38 mmol, 1.0 eq.) in THF (10 mL) was added LiHMDS (076 mL, 1 M in THF, 0.76 mmol, 2.0 eq.) dropwise at −60° C. under N₂ and the resulting solution was stirred at −60° C. for 20 min under N₂. POCl₃ (116 mg, 0.76 mmol, 2.0 eq.) in THF (10 mL) was quickly added and the resulting mixture was stirred at −60° C. for 15 min. 2-Bromoethylamine hydrobromide (540 mg, 2.66 mmol, 7.0 eq.) and TEA (461 mg, 4.56 mmol, 12.0 eq.) were added and the mixture was stirred at −60° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 6-(4-(dimethylcarbamoyl)phenoxy)-5-nitro-2,3-dihydro-1H-inden-1-yl di((2-bromoethyl)amino)phosphinate (55 mg, 87 μmol, 23%) as a yellow oil. LC-MS (ESI): m/z 633.1 [M+H]⁺.

((R)-6-(4-(dimethylcarbamoyl)phenoxy)-5-nitro-2,3-dihydro-1H-inden-1-yl di(aziridin-1-yl)phosphinate & ((S)-6-(4-(dimethylcarbamoyl)phenoxy)-5-nitro-2,3-dihydro-1H-inden-1-yl di(aziridin-1-yl)phosphinate

To a solution of 6-(4-(dimethylcarbamoyl)phenoxy)-5-nitro-2,3-dihydro-1H-inden-1-yl di((2-bromoethyl)amino)phosphinate (55 mg, 87 μmol, 1.0 eq.) in THF (5 mL) were added Ag₂O (202 mg, 0.87 mmol, 10.0 eq.) and DIEA (112 mg, 0.87 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 6-(4-(dimethylcarbamoyl)phenoxy)-5-nitro-2,3-dihydro-1H-inden-1-yl di(aziridin-1-yl)phosphinate (24 mg, 51 μmol, 59%) as a solid in a stereo isomeric mixture form, which was further separated by Chiral SFC to give:

Isomer 1 (140), 9.5 mg, 40%, Retention time: 4.437 min, >99% ee. LC-MS (ESI): m/z 473.2 [M+H]⁺; ¹H NMR (400 MHz, CDCl₃) δ 7.84 (s, 1H), 7.45-7.42 (m, 2H), 7.26 (s, 1H), 7.04-7.00 (m, 2H), 5.96-5.91 (m, 1H), 3.19-3.12 (m, 1H), 3.10-3.03 (m, 6H), 2.96-2.88 (m, 1H), 2.67-2.59 (m, 1H), 2.35-2.27 (m, 1H), 2.20-2.07 (m, 8H). ³¹P NMR (162 MHz, CDCl₃) δ 30.01 (s).

Isomer 2 (141), 8.0 mg, 33%, Retention time: 3.907 min, >99% ee. LC-MS (ESI): m/z 473.2 [M+H]⁺; ¹H NMR (400 MHz, CDCl₃) δ 7.83 (s, 1H), 7.47-7.39 (m, 2H), 7.05-6.98 (m, 2H), 7.26 (s, 1H), 5.95-5.91 (m, 1H), 3.22-3.13 (m, 1H), 3.12-2.99 (m, 6H), 2.96-2.86 (m, 1H), 2.70-2.56 (m, 1H), 2.38-2.25 (m, 1H), 2.22-2.04 (m, 8H). ³¹P NMR (162 MHz, CDCl₃) δ 30.01 (s).

Analytical method: Column: ChiralPak IA, 250×4.6 mm I.D., 5 μm, Mobile phase: A for CO₂ and B for EtOH (0.05% DEA), Gradient: 10 min @ 40%, Flow rate: 2.0 mL/min, Back pressure: 100 bar, Column temperature: 35° C.

SFC Method: Instrument: SHIMADZU PREP SOLUTION SFC, Column: ChiralPak AD, 250×21.2 mm I.D., 5 μm, Mobile phase: A for CO₂ and B for ETOH+0.1% NH₃H₂O, Gradient: B 30%, Flow rate: 40 mL/min, Back pressure: 100 bar, Column temperature: 35° C., Wavelength: 220 nm, Cycle-time: 5 min, Eluted time: 2 h.

### Illustration 21. Synthesis of (R)-6-(4-(methylcarbamoyl)phenoxy)-5-nitro-2,3-dihydro-1H-inden-1-yl di(aziridin-1-yl)phosphinate & (S)-6-(4-(methylcarbamoyl)phenoxy)-5-nitro-2,3-dihydro-1H-inden-1-yl di(aziridin-1-yl)phosphinate (150&151) (General Procedure 9, 6,5 core 4-method B)

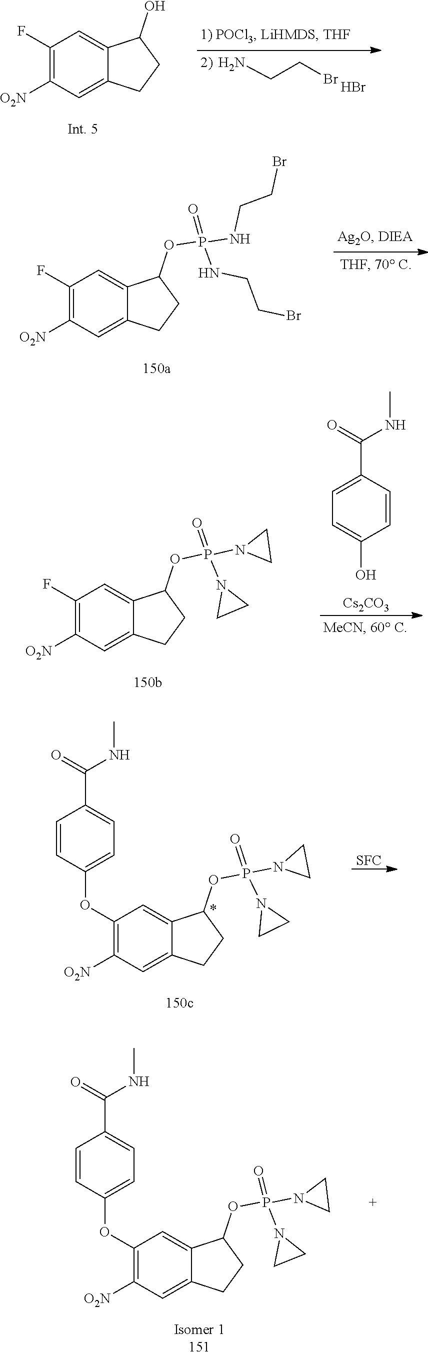

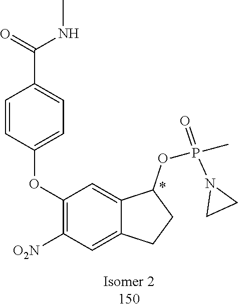 [SMILES: CNC(=O)c1ccc(Oc2cc3c(cc2[N+](=O)[O-])CCC3OP(C)(=O)N2CC2)cc1]

6-fluoro-5-nitro-2,3-dihydro-1H-inden-1-yl di((2-bromoethyl)amino)phosphinate

To a solution of 6-fluoro-5-nitro-2,3-dihydro-1H-inden-1-ol (50 mg, 0.25 mmol, 1.0 eq.) in THF (5 mL) was added LiHMDS (0.28 mL, 1 M in THF, 0.28 mmol, 1.1 eq.) dropwise at −65° C. under N₂ and the resulting solution was stirred at −65° C. for 20 min under N₂. POCl₃ (78 mg, 0.51 mmol, 2.0 eq.) was added and the resulting mixture was stirred at −65° C. for 20 min. 2-Bromoethylamine hydrobromide (312 mg, 1.52 mmol, 6.0 eq.) and TEA (0.42 mL, 3.05 mmol, 12.0 eq.) were added into the above mixture. The resulting mixture was warmed up to room temperature and stirred for 1 hr. After completion, the reaction mixture was quenched with NH₄Cl solution (10 mL, sat., aq.) and extracted with EtOAc (15 mL×2). The organic layers were combined and washed with brine (15 mL), dried over anhydrous Na₂SO₄ and concentrated under reduced pressure. The residue was purified by flash column chromatography on silica gelt to afford 6-fluoro-5-nitro-2,3-dihydro-1H-inden-1-yl di((2-bromoethyl)amino)phosphinate (32 mg, 65 μmol, 26%) as a yellow oil. LC-MS (ESI): m/z 488 [M+H]⁺.

6-fluoro-5-nitro-2,3-dihydro-1H-inden-1-yl di(aziridin-1-yl)phosphinate

To a solution of 6-fluoro-5-nitro-2,3-dihydro-1H-inden-1-yl di((2-bromoethyl)amino)phosphinate (100 mg, 0.21 mmol, 1.0 eq.) in THF (5 mL) were added Ag₂O (487 mg, 2.10 mmol, 10.0 eq.) and DIEA (271 mg, 2.10 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 6-fluoro-5-nitro-2,3-dihydro-1H-inden-1-yl di(aziridin-1-yl)phosphinate (60 mg, 0.17 mmol, 59%) as a solid. LC-MS (ESI): m/z 328 [M+H]⁺.

(R)-6-(4-(methylcarbamoyl)phenoxy)-5-nitro-2,3-dihydro-1H-inden-1-yl di(aziridin-1-yl)phosphinate & (S)-6-(4-(methylcarbamoyl)phenoxy)-5-nitro-2,3-dihydro-1H-inden-1-yl di(aziridin-1-yl)phosphinate

To a solution of 6-fluoro-5-nitro-2,3-dihydro-1H-inden-1-yl di(aziridin-1-yl)phosphinate (80 mg, 0.24 mmol, 1.0 eq.) and 4-hydroxy-N-methylbenzamide (54 mg, 0.36 mmol, 1.5 eq.) in MeCN (10 mL) was added Cs₂CO₃ (157 mg, 0.48 mmol, 2.0 eq.). The reaction mixture was stirred at 60° C. for 2 hrs. After completion, the reaction mixture was concentrated under reduced pressure. The residue was purified by Biotage® C18 column chromatography to give 6-(4-(methylcarbamoyl)phenoxy)-5-nitro-2,3-dihydro-1H-inden-1-yl di(aziridin-1-yl)phosphinate (34 mg, 74 μmol, 31%) as a white solid in a stereo isomeric mixture form. 6-(4-(methylcarbamoyl)phenoxy)-5-nitro-2,3-dihydro-1H-inden-1-yl di(aziridin-1-yl)phosphinate (30 mg, 66 μmol) was further separated by Chiral SFC to give:

Isomer 1 (151), 11.6 mg, 25 μmol, 38%, Retention time: 3.275 min, >99% ee. LC-MS (ESI): m/z 459.1 [M+H]⁺; ¹H NMR (400 MHz, CDCl₃) δ 7.78 (s, 1H), 7.71-7.67 (m, 2H), 7.21 (s, 1H), 6.97-6.93 (m, 2H), 6.13-6.03 (m, 1H), 5.89-5.84 (m, 1H), 3.13-3.06 (m, 1H), 2.94 (d, J=4.8 Hz, 3H), 2.89-2.82 (m, 1H), 2.61-2.52 (m, 1H), 2.28-2.20 (m, 1H), 2.14-2.12 (m, 1H), 2.10-1.97 (m, 7H). ³¹P NMR (162 MHz, CDCl₃) δ 29.98 (s).

Isomer 2 (150), 16.8 mg, 37 μmol, 56%, Retention time: 3.897 min, 99% ee. LC-MS (ESI): m/z 459.1 [M+H]⁺; ¹H NMR (400 MHz, CDCl₃) δ 7.78 (s, 1H), 7.72-7.66 (m, 2H), 7.21 (s, 1H), 6.98-6.91 (m, 2H), 6.14-6.04 (m, 1H), 5.89-5.84 (m, 1H), 3.15-3.04 (m, 1H), 2.94 (d, J=4.8 Hz, 3H), 2.89-2.83 (m, 1H), 2.59-2.54 (m, 1H), 2.29-2.21 (m, 1H), 2.15-2.11 (m, 1H), 2.10-1.98 (m, 7H). ³¹P NMR (162 MHz, CDCl₃) δ 30.00 (s).

Analytical method: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C.

SFC Method: Instrument: Waters Thar 80 preparative SFC, Column: ChiralPak AD, 250×21.2 mm I.D., 5 μm, Mobile phase: A for CO₂ and B for MEOH+0.1% NH₃H₂O, Gradient: B 30%, Flow rate: 40 mL/min, Back pressure: 100 bar, Column temperature: 35° C., Wavelength: 220 nm, Cycle-time: 15 min, Eluted time: 2 H.

### Illustration 22. Synthesis of (R)-6-([1,1′-biphenyl]-4-yloxy)-5-nitro-2,3-dihydro-1H-inden-1-yl di(aziridin-1-yl)phosphinate & (S)-6-([1,1′-biphenyl]-4-yloxy)-5-nitro-2,3-dihydro-1H-inden-1-yl di(aziridin-1-yl)phosphinate (152&153)

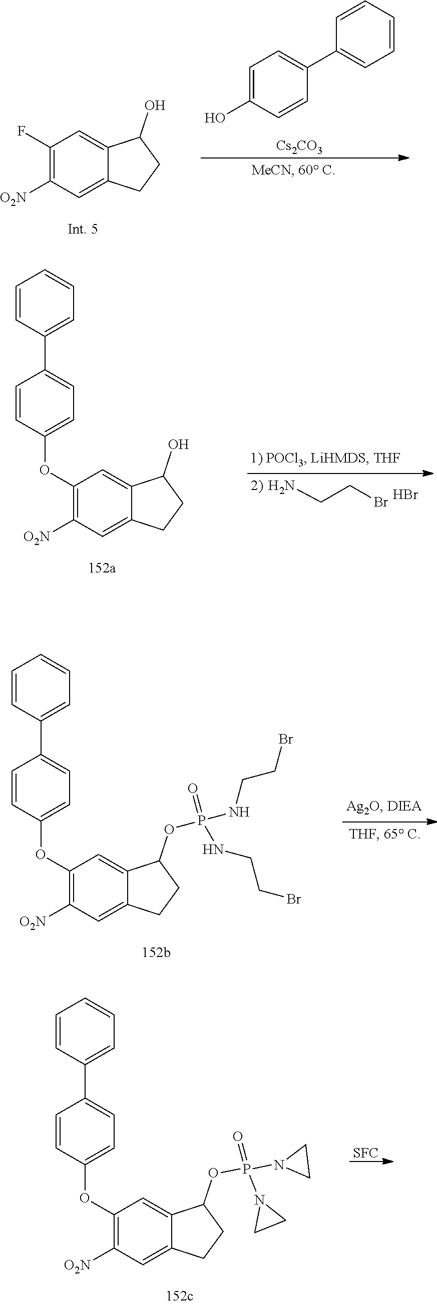

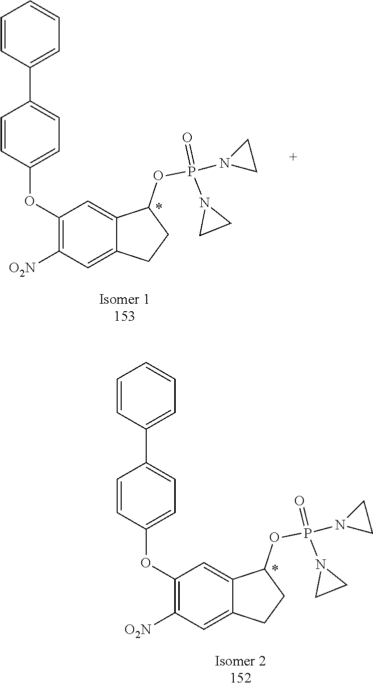 [SMILES: O=[N+]([O-])c1cc2c(cc1Oc1ccc(-c3ccccc3)cc1)C(OP(=O)(N1CC1)N1CC1)CC2 ; O=[N+]([O-])c1cc2c(cc1Oc1ccc(-c3ccccc3)cc1)C(OP(=O)(N1CC1)N1CC1)CC2]

6-([1,1′-biphenyl]-4-yloxy)-5-nitro-2,3-dihydro-1H-inden-1-ol

To a solution of 6-fluoro-5-nitro-2,3-dihydro-1H-inden-1-ole (750 mg, 3.80 mmol 1.0 eq.) and [1,1′-biphenyl]-4-ol (842 mg, 4.95 mmol, 1.3 eq.) in ACN (5 mL) was added Cs₂CO₃ (2.48 g, 7.61 mmol, 2.0 eq.). The reaction mixture was stirred at 60° C. for 1 hr. After completion, the reaction mixture was concentrated under reduced pressure. The residue was purified by Biotage® C18 column chromatography to give 6-([1,1′-biphenyl]-4-yloxy)-5-nitro-2,3-dihydro-1H-inden-1-ol (650 mg, 1.87 mmol, 49%) as a yellow solid. LC-MS (ESI): m/z 348 [M+H]⁺.

([1,1′-biphenyl]-4-yloxy)-5-nitro-2,3-dihydro-1H-inden-1-yl di((2-bromoethyl)amino)phosphinate

To a solution of 6-([1,1′-biphenyl]-4-yloxy)-5-nitro-2,3-dihydro-1H-inden-1-ol (650 mg, 1.87 mmol, 1.0 eq.) in THF (10 mL) was added LiHMDS (2.25 mL, 1 M in THE, 2.25 mmol, 1.2 eq.) dropwise at −65° C. under N₂ and the resulting solution was stirred at −65° C. for 20 min under N₂. POCl₃ (0.35 mL, 3.74 mmol, 2.0 eq.) in THF (10 mL) was quickly added and the resulting mixture was stirred at −65° C. for 15 min. 2-Bromoethylamine hydrobromide (2.69 g, 13.1 mmol, 7.0 eq.) and TEA (2.27 g, 22.4 mmol, 12.0 eq.) were added and the mixture was stirred at −65° C. for 10 min. The resulting mixture was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 6-([1,1′-biphenyl]-4-yloxy)-5-nitro-2,3-dihydro-1H-inden-1-yl di((2-bromoethyl)amino)phosphinate (465 mg, 0.73 mmol, 39%) as a yellow oil. LC-MS (ESI): m/z 638 [M+H]⁺.

(R)-6-([1,1′-biphenyl]-4-yloxy)-5-nitro-2,3-dihydro-1H-inden-1-yl di(aziridin-1-yl)phosphinate & (S)-6-([1,1′-biphenyl]-4-yloxy)-5-nitro-2,3-dihydro-1H-inden-1-yl di(aziridin-1-yl)phosphinate

To a solution of 6-([1,1′-biphenyl]-4-yloxy)-5-nitro-2,3-dihydro-1H-inden-1-yl di((2-bromoethyl)amino)phosphinate (465 mg, 0.73 mmol, 1.0 eq.) in THF (10 mL) were added Ag₂O ((843 mg, 3.64 mmol, 5.0 eq.) and DIEA (470 mg, 3.64 mmol, 5.0 eq.). The resulting solution was stirred at 65° C. for 18 hrs under N₂. After completion, the reaction mixture was filtered and the filtrate was concentrated under reduced pressure. The residue was purified by flash column chromatography on silica gel to afford 6-([1,1′-biphenyl]-4-yloxy)-5-nitro-2,3-dihydro-1H-inden-1-yl di(aziridin-1-yl)phosphinate as a yellow solid in a stereo isomeric mixture form, which was further separated by Chiral SFC to give:

Isomer 1 (153), 109.8 mg, 0.23 mmol, 32%, Retention time: 3.708 min, 95% ee. LC-MS (ESI): m/z 478.2 [M+H]⁺; ¹H NMR (400 MHz, CDCl₃) δ 7.82 (s, 1H), 7.60-7.54 (m, 4H), 7.46-7.42 (m, 2H), 7.37-7.33 (m, 1H), 7.23 (s, 1H), 7.13-7.08 (m, 2H), 5.95-5.90 (m, 1H), 3.18-3.10 (m, 1H), 2.94-2.86 (m, 1H), 2.66-2.57 (m, 1H), 2.33-2.24 (m, 1H), 2.17-2.16 (m, 1H), 2.14-2.01 (m, 7H). ³¹P NMR (162 MHz, CDCl₃) δ 29.98 (s).

Isomer 2 (152), 90.9 mg, 0.19 mmol, 26%, Retention time: 4.126 min, 98% ee. LC-MS (ESI): m/z 478.2 [M+H]⁺; ¹H NMR (400 MHz, CDCl₃) δ 7.82 (s, 1H), 7.59-7.54 (m, 4H), 7.46-7.42 (m, 2H), 7.37-7.33 (m, 1H), 7.23 (s, 1H), 7.12-7.10 (m, 2H), 5.95-5.90 (m, 1H), 3.18-3.10 (m, 1H), 2.94-2.86 (m, 1H), 2.66-2.57 (m, 1H), 2.32-2.24 (m, 1H), 2.17-2.15 (m, 1H), 2.13-2.00 (m, 7H). ³¹P NMR (162 MHz, CDCl₃) δ 29.98 (s).

Analytical method: Column: ChiralCel OJ, 250×4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C.

SFC Method: Instrument: Waters Thar 80 preparative SFC, Column: ChiralCel OD, 250×21.2 mm I.D., 5 μm, Mobile phase: A for CO₂ and B for MEOH, Gradient: B 30%, Flow rate: 40 mL/min, Back pressure: 100 bar, Column temperature: 35° C., Wavelength: 220 nm, Cycle-time: 20 min, Eluted time: 5 H.

### Illustration 23. Synthesis of (S)-6-([1,1′-biphenyl]-3-yloxy)-5-nitro-2,3-dihydro-1H-inden-1-yl di(aziridin-1-yl)phosphinate & (R)-6-([1,1′-biphenyl]-3-yloxy)-5-nitro-2,3-dihydro-1H-inden-1-yl di(aziridin-1-yl)phosphinate (168&169)

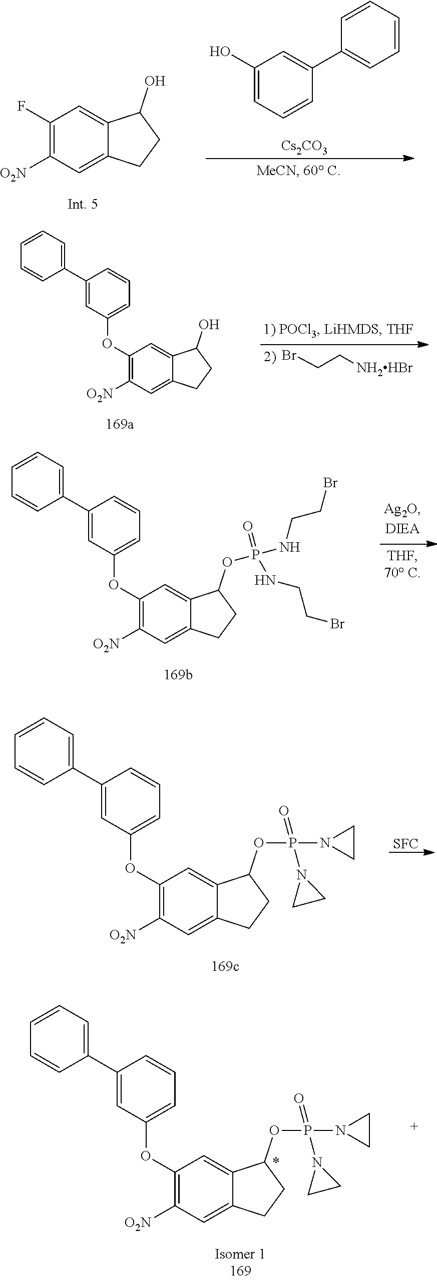

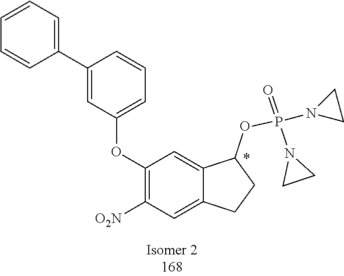 [SMILES: O=[N+]([O-])c1cc2c(cc1Oc1cccc(-c3ccccc3)c1)C(OP(=O)(N1CC1)N1CC1)CC2]

6-([1,1′-biphenyl]-3-yloxy)-5-nitro-2,3-dihydro-1H-inden-1-ol

To a solution of 6-fluoro-5-nitro-2,3-dihydro-1H-inden-1-ol (400 mg, 2.03 mmol, 1.0 eq.) and [1,1′-biphenyl]-3-ol (414 mg, 2.44 mmol, 1.4 eq.) in ACN (10 mL) was added Cs₂CO₃ (1.32 g, 4.06 mmol, 2.0 eq.). The reaction mixture was stirred at 60° C. for 1 hr. After completion, the reaction mixture was concentrated under reduced pressure. The residue was purified by Biotage® C18 column chromatography to give 6-([1,1′-biphenyl]-3-yloxy)-5-nitro-2,3-dihydro-1H-inden-1-ol (522 mg, 1.50 mmol, 74%) as a yellow solid. LCMS (ESI): m/z 348 [M+H]⁺.

6-([1,1′-biphenyl]-3-yloxy)-5-nitro-2,3-dihydro-1H-inden-1-yl di((2-bromoethyl)amino)phosphinate

To a solution of 6-([1,1′-biphenyl]-3-yloxy)-5-nitro-2,3-dihydro-1H-inden-1-ol (200 mg, 0.58 mmol, 1.0 eq.) in THF (30 mL) was added LiHMDS (0.69 mL, 1 M in THF, 0.69 mmol, 1.2 eq.) at −78° C. under N₂ and the resulting solution was stirred at −78° C. for 15 min under N₂. POCl₃ (0.11 mL, 1.15 mmol, 2.0 eq.) was added and the resulting mixture was stirred at −78° C. for 15 min. 2-Bromoethylamine hydrobromide (708 mg, 3.46 mmol, 6.0 eq.) and TEA (0.96 mL, 6.91 mmol, 12.0 eq.) were added. The mixture was warmed up to room temperature and stirred for 1 hr. After completion, the reaction mixture was quenched with NH₄Cl solution (10 mL, sat., aq.) and extracted with EtOAc (20 mL×2). The organic layers were combined and washed with brine (10 mL), dried over anhydrous Na₂SO₄ and concentrated under reduced pressure. The residue was purified by Biotage® C18 column chromatography to afford 6-([1,1′-biphenyl]-3-yloxy)-5-nitro-2,3-dihydro-1H-inden-1-yl di((2-bromoethyl)amino)phosphinate (150 mg, 0.24 mmol, 41%) as a white solid. LCMS (ESI): m/z 638 [M+H]⁺.

6-([1,1′-biphenyl]-3-yloxy)-5-nitro-2,3-dihydro-1H-inden-1-yl di(aziridin-1-yl)phosphinate

To a solution of 6-([1,1′-biphenyl]-3-yloxy)-5-nitro-2,3-dihydro-1H-inden-1-yl di((2-bromoethyl)amino)phosphinate (150 mg, 0.24 mmol, 1.0 eq.) inTHF (30 mL) were added Ag₂O (544 mg, 2.35 mmol, 10.0 eq.) and DIEA (0.39 mL, 2.35 mmol, 10.0 eq.). The resulting solution was stirred at 70° C. overnight under N₂. After completion, the reaction mixture was filtered and the filtrate was concentrated under reduced pressure. The residue was purified by Biotage® C18 column chromatography to afford 6-([1,1′-biphenyl]-3-yloxy)-5-nitro-2,3-dihydro-1H-inden-1-yl di(aziridin-1-yl)phosphinate (100 mg, 0.21 mmol, 89%) as a yellow oil. ¹H NMR (400 MHz, CDCl₃) δ 7.81 (s, 1H), 7.58-7.53 (m, 2H), 7.46-7.39 (m, 4H), 7.38-7.33 (m, 1H), 7.28-7.26 (m, 1H), 7.21 (s, 1H), 7.04-7.01 (m, 1H), 5.94-5.87 (m, 1H), 3.17-3.09 (m, 1H), 2.94-2.84 (m, 1H), 2.65-2.55 (m, 1H), 2.29-2.23 (m, 1H), 2.14-1.96 (m, 8H). ³¹P NMR (162 MHz, CDCl₃) δ 29.88 (s). LC-MS (ESI): m/z 478 [M+H]⁺.

(S)-6-([1,1′-biphenyl]-3-yloxy)-5-nitro-2,3-dihydro-1H-inden-1-yl di(aziridin-1-yl)phosphinate & (R)-6-([1,1′-biphenyl]-3-yloxy)-5-nitro-2,3-dihydro-1H-inden-1-yl di(aziridin-1-yl)phosphinate

6-([1,1′-biphenyl]-3-yloxy)-5-nitro-2,3-dihydro-1H-inden-1-yl di(aziridin-1-yl)phosphinate (200 mg, 0.42 mmol) was purified by Chiral SFC to give:

Isomer 1 (169), 86.96 mg, 0.18 mmol, 43%, Retention time: 1.618 min, >99% ee. LC-MS (ESI): m/z 478.1 [M+H]⁺; ¹H NMR (400 MHz, CDCl₃) δ 7.78 (s, 1H), 7.52 (d, J=7.4 Hz, 2H), 7.44-7.36 (m, 4H), 7.34-7.28 (m, 1H), 7.25 (s, 1H), 7.19 (s, 1H), 7.02-6.97 (m, 1H), 5.91-5.84 (m, 1H), 3.15-3.05 (m, 1H), 2.92-2.81 (m, 1H), 2.62-2.52 (m, 1H), 2.29-2.20 (m, 1H), 2.10-1.91 (m, 8H). ³¹P NMR (162 MHz, CDCl₃) δ 29.86 (s).

Isomer 2 (168), 102.02 mg, 0.21 mmol, 50%, Retention time: 1.950 min, 96% ee. LC-MS (ESI): m/z 478.1 [M+H]⁺; ¹H NMR (400 MHz, CDCl₃) δ 7.81 (s, 1H), 7.55 (d, J=7.4 Hz, 2H), 7.46-7.39 (m, 4H), 7.37-7.31 (m, 1H), 7.27 (s, 1H), 7.22 (s, 1H), 7.05-7.00 (m, 1H), 5.94-5.85 (m, 1H), 3.18-3.08 (m, 1H), 2.94-2.83 (m, 1H), 2.66-2.54 (m, 1H), 2.32-2.23 (m, 1H), 2.13-1.96 (m, 8H). ³¹P NMR (162 MHz, CDCl₃) δ 29.86 (s).

Analytical method: Column: ChiralPak IH, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

SFC Method: Instrument: Waters Thar 80 preparative SFC, Column: ChiralPak IH, 250×21.2 mm I.D., 5 μm, Mobile phase: A for CO₂ and B for MEOH+0.1% NH₃H₂O, Gradient: B 35%, Flow rate: 40 mL/min, Back pressure: 100 bar, Column temperature: 35° C., Wavelength: 220 nm, Cycle-time: 10 min, Eluted time: 2 H.

### Illustration 24. Synthesis of (R)-6-((2-cyclopropyl-3-oxoisoindolin-5-yl)oxy)-5-nitro-2,3-dihydro-1H-inden-1-yl di(aziridin-1-yl)phosphinate & (S)-6-((2-cyclopropyl-3-oxoisoindolin-5-yl)oxy)-5-nitro-2,3-dihydro-1H-inden-1-yl di(aziridin-1-yl)phosphinate (170&171)

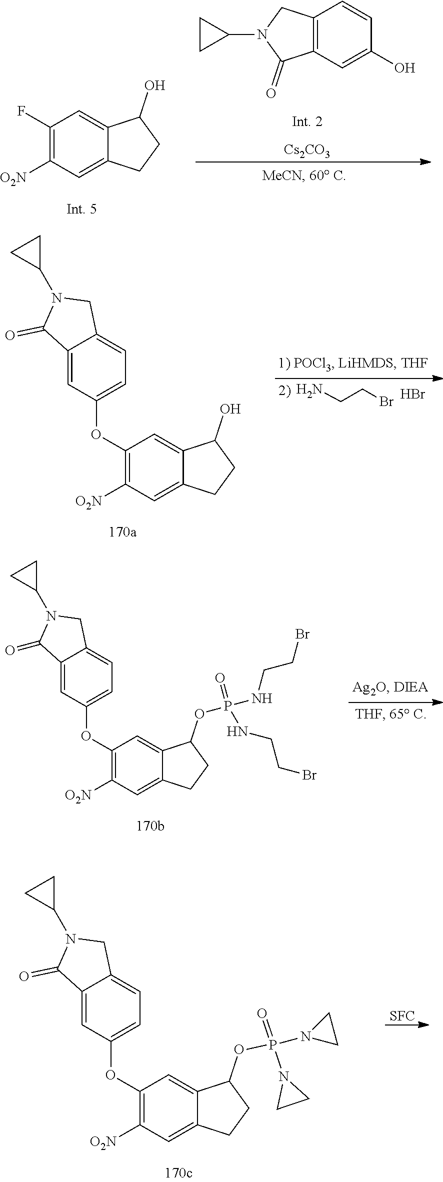

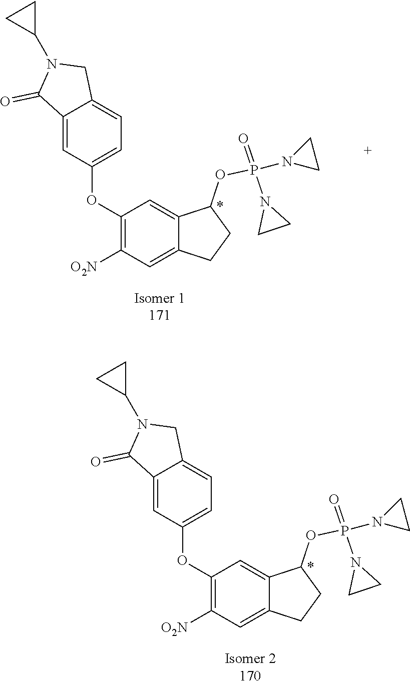 [SMILES: O=C1c2cc(Oc3cc4c(cc3[N+](=O)[O-])CCC4OP(=O)(N3CC3)N3CC3)ccc2CN1C1CC1 ; O=C1c2cc(Oc3cc4c(cc3[N+](=O)[O-])CCC4OP(=O)(N3CC3)N3CC3)ccc2CN1C1CC1]

2-cyclopropyl-6-((3-hydroxy-6-nitro-2,3-dihydro-1H-inden-5-yl)oxy) isoindolin-1-one

To a solution of 6-fluoro-5-nitro-2,3-dihydro-1H-inden-1-ole (130 mg, 0.66 mmol, 1.0 eq.) and 2-cyclopropyl-6-hydroxyisoindolin-1-one (162 mg, 0.86 mmol, 1.3 eq.) in ACN (5 mL) was added Cs₂CO₃ (430 mg, 1.32 mmol, 2.0 eq.). The reaction mixture was stirred at 60° C. for 2 hrs. After completion, the reaction mixture was concentrated under reduced pressure. The residue was purified by Biotage® C18 column chromatography to afford 2-cyclopropyl-6-((3-hydroxy-6-nitro-2,3-dihydro-1H-inden-5-yl)oxy) isoindolin-1-one (140 mg, 0.38 mmol, 58%) as a colorless oil. LC-MS (ESI): m/z 367 [M+H]⁺.

6-((2-cyclopropyl-3-oxoisoindolin-5-yl)oxy)-5-nitro-2,3-dihydro-1H-inden-1-yl di((2-bromoethyl)amino)phosphinate

To a solution of 2-cyclopropyl-6-((3-hydroxy-6-nitro-2,3-dihydro-1H-inden-5-yl)oxy)isoindolin-1-one (90 mg, 0.25 mmol, 1.0 eq.) in THF (20 mL) was added LiHMDS (0.30 mL, 1 M in THF, 0.30 mmol, 1.2 eq.) dropwise at −65° C. under N₂ and the resulting solution was stirred at −65° C. for 30 min under N₂. POCl₃ (75 mg, 0.49 mmol, 2.0 eq.) in THF (10 mL) was quickly added and the resulting mixture was stirred at −65° C. for 15 min. 2-Bromoethylamine hydrobromide (312 mg, 1.52 mmol, 6.0 eq.) and TEA (298 mg, 2.95 mmol, 12.0 eq.) were added and the mixture was stirred at −65° C. for 30 min. The resulting mixture was warmed up to room temperature and stirred for 1 hr. After completion, the reaction mixture was quenched with NH₄Cl (10 mL, sat., aq.) and extracted with EtOAc (15 mL×2). The organic layers were combined and washed with brine (5 mL), dried over anhydrous Na₂SO₄ and concentrated under reduced pressure. The residue was purified by flash column chromatography on silica gel to afford 6-((2-cyclopropyl-3-oxoisoindolin-5-yl)oxy)-5-nitro-2,3-dihydro-1H-inden-1-yl di((2-bromoethyl)amino)phosphinate (110 mg, 0.17 mmol, 68%) as a yellow oil. LC-MS (ESI): m/z 657 [M+H]⁺.

(R)-6-((2-cyclopropyl-3-oxoisoindolin-5-yl)oxy)-5-nitro-2,3-dihydro-1H-inden-1-yl di(aziridin-1-yl)phosphinate & (S)-6-((2-cyclopropyl-3-oxoisoindolin-5-yl)oxy)-5-nitro-2,3-dihydro-1H-inden-1-yl di(aziridin-1-yl)phosphinate

To a solution of 6-((2-cyclopropyl-3-oxoisoindolin-5-yl)oxy)-5-nitro-2,3-dihydro-1H-inden-1-yl di((2-bromoethyl)amino)phosphinate (30 mg, 45.7 μmol, 1.0 eq.) in THF (10 mL) were added Ag₂O (53 mg, 0.23 mmol, 5.0 eq.) and DIEA (30 mg, 0.23 mmol, 5.0 eq.). The resulting solution was stirred at 65° C. for 18 hrs under N₂. After completion, the reaction mixture was filtered and the filtrate was concentrated under reduced pressure. The residue was purified by flash column chromatography on silica gel to afford 6-((2-cyclopropyl-3-oxoisoindolin-5-yl)oxy)-5-nitro-2,3-dihydro-1H-inden-1-yl di(aziridin-1-yl)phosphinate as a yellow solid in a stereo isomeric mixture form, which was further separated by Chiral SFC to give:

Isomer 1 (171), 11.9 mg, 24 μmol, 14%, Retention time: 4.019 min, 99% ee. LC-MS (ESI): m/z 497.1 [M+H]⁺; ¹H NMR (400 MHz, DMSO) δ 8.03 (s, 1H), 7.61 (d, J=8.2 Hz, 1H), 7.39-7.31 (m, 1H), 7.21 (s, 1H), 7.14-7.08 (m, 1H), 5.95-5.72 (m, 1H), 4.39 (s, 2H), 3.12-3.03 (m, 1H), 2.97-2.84 (m, 2H), 2.61-2.53 (m, 1H), 2.21-2.10 (m, 1H), 2.08-1.91 (m, 8H), 0.86-0.76 (m, 4H). ³¹P NMR (162 MHz, DMSO) δ 29.90 (s).

Isomer 2 (170), 12.6 mg, 25 μmol, 15%, Retention time: 4.426 min, 92% ee. LC-MS (ESI): m/z 497.1 [M+H]⁺; ¹H NMR (400 MHz, DMSO) δ 8.03 (s, 1H), 7.61 (d, J=8.2 Hz, 1H), 7.36 (dd, J=8.2, 2.3 Hz), 7.21 (s, 1H), 7.13 (d, J=2.4 Hz, 1H), 5.87-5.82 (m, 1H), 4.40 (s, 2H), 3.10-3.04 (m, 1H), 2.95-2.89 (m, 2H), 2.57-2.54 (m, 1H), 2.16-2.11 (m, 1H), 2.08-1.93 (m, 8H), 0.83-0.77 (m, 4H). ³¹P NMR (162 MHz, CDCl₃) δ 29.90 (s).

Analytical method: Column: ChiralPak IH, 100×4.6 mm I.D., 5 μm; Mobile phase: A for CO₂ and B for methanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C.

SFC Method: Instrument: Waters Thar 80 preparative SFC, Column: ChiralPak IH, 250×21.2 mm I.D., 5 μm, Mobile phase: A for CO₂ and B for MEOH+0.1% NH₃H₂O, Gradient: B 35%, Flow rate: 40 mL/min, Back pressure: 100 bar, Column temperature: 35° C., Wavelength: 220 nm, Cycle-time: 10 min, Eluted time: 2 H.

### Illustration 25. Synthesis of (R)-6-(4-(cyclopropylcarbamoyl)phenoxy)-5-nitro-2,3-dihydro-1H-inden-1-yl di(aziridin-1-yl)phosphinate & (S)-6-(4-(cyclopropylcarbamoyl)phenoxy)-5-nitro-2,3-dihydro-1H-inden-1-yl di(aziridin-1-yl)phosphinate (172&173)

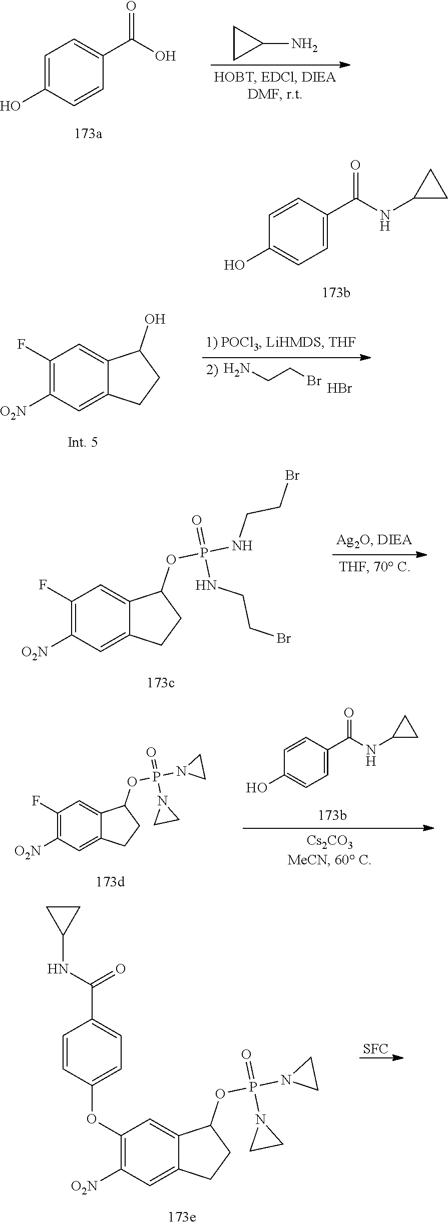

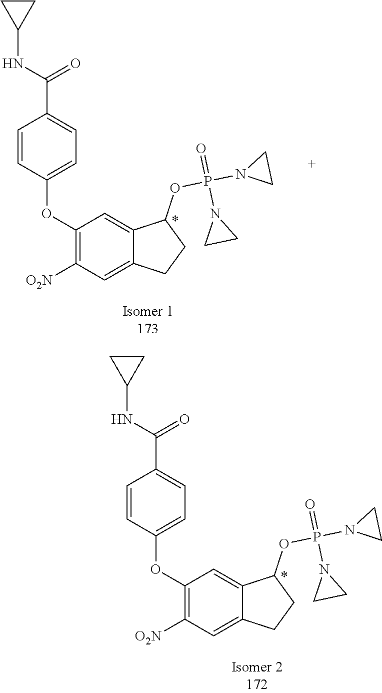 [SMILES: O=C(NC1CC1)c1ccc(Oc2cc3c(cc2[N+](=O)[O-])CCC3OP(=O)(N2CC2)N2CC2)cc1 ; O=C(NC1CC1)c1ccc(Oc2cc3c(cc2[N+](=O)[O-])CCC3OP(=O)(N2CC2)N2CC2)cc1]

N-cyclopropyl-4-hydroxybenzamide

To a solution of 4-hydroxybenzoic acid (0.69 mL, 7.24 mmol, 1.0 eq.) and cyclopropanamine (0.50 mL, 7.24 mmol, 1.0 eq.) in DMF (5 mL) was added HOBt (0.98 g, 7.24 mmol, 1.0 eq.) and EDCI (1.39 g, 7.24 mmol, 1.0 eq.). The mixture was stirred at room temperature overnight. After completion, the reaction mixture was filtered and the filtrates were concentrated under reduced pressure. The residue was purified by flash column chromatography on silica gel to afford N-cyclopropyl-4-hydroxybenzamide (5, 500 mg, 2.82 mmol, 39%) as a yellow solid. LCMS (ESI): m/z 178.0 [M+H]⁺. ¹H NMR (400 MHz, DMSO-d₆) δ 9.91 (s, 1H), 8.15 (d, J=3.5 Hz, 1H), 7.72-7.61 (m, 2H), 6.77 (d, J=8.5 Hz, 2H), 2.83-2.71 (m, 1H), 0.72-0.57 (m, 2H), 0.57-0.47 (m, 2H).

6-fluoro-5-nitro-2,3-dihydro-1H-inden-1-yl di((2-bromoethyl)amino)phosphinate

To a solution of 6-fluoro-5-nitro-2,3-dihydro-1H-inden-1-ol (50 mg, 0.25 mmol, 1.0 eq.) in THF (5 mL) was added LiHMDS (0.28 mL, 1 M in THF, 0.28 mmol, 1.1 eq.) dropwise at −65° C. under N₂ and the resulting solution was stirred at −65° C. for 20 min under N₂. POCl₃ (78 mg, 0.51 mmol, 2.0 eq.) was added and the resulting mixture was stirred at −65° C. for 20 min. 2-Bromoethylamine hydrobromide (312 mg, 1.52 mmol, 6.0 eq.) and TEA (0.42 mL, 3.05 mmol, 12.0 eq.) were added into the above mixture. The resulting mixture was warmed up to room temperature and stirred for 1 hr. After completion, the reaction mixture was quenched with NH₄Cl solution (10 mL, sat., aq.) and extracted with EtOAc (15 mL×2). The organic layers were combined and washed with brine (15 mL), dried over anhydrous Na₂SO₄ and concentrated under reduced pressure. The residue was purified by flash column chromatography on silica gelt to afford 6-fluoro-5-nitro-2,3-dihydro-1H-inden-1-yl di((2-bromoethyl)amino)phosphinate (32 mg, 65 μmol, 26%) as a yellow oil. LCMS (ESI): m/z 488 [M+H]⁺.

6-fluoro-5-nitro-2,3-dihydro-1H-inden-1-yl di(aziridin-1-yl)phosphinate

To a solution of 6-fluoro-5-nitro-2,3-dihydro-1H-inden-1-yl di((2-bromoethyl)amino)phosphinate (100 mg, 0.21 mmol, 1.0 eq.) in THF (5 mL) were added Ag₂O (487 mg, 2.10 mmol, 10.0 eq.) and DIEA (271 mg, 2.10 mmol, 10.0 eq.). The resulting solution was stirred at 70° C. for 18 hrs under N₂. After completion, the reaction mixture was filtered and the filtrate was concentrated under reduced pressure. The residue was purified by Biotage® C18 column chromatography to afford 6-fluoro-5-nitro-2,3-dihydro-1H-inden-1-yl di(aziridin-1-yl)phosphinate (60 mg, 0.17 mmol, 59%) as a solid. LCMS (ESI): m/z 328 [M+H]⁺.

6-(4-(cyclopropylcarbamoyl)phenoxy)-5-nitro-2,3-dihydro-1H-inden-1-yl di(aziridin-1-yl)phosphinate

To a solution of 6-fluoro-5-nitro-2,3-dihydro-1H-inden-1-yl di(aziridin-1-yl)phosphinate (68 mg, 0.21 mmol, 1.0 eq.) and N-cyclopropyl-4-hydroxybenzamide (56 mg, 0.32 mmol, 1.5 eq.) in MeCN (10 mL) was added Cs₂CO₃ (203 mg, 0.62 mmol, 3.0 eq.). The reaction mixture was stirred at 60° C. overnight. After completion, the reaction mixture was diluted with H₂O (10 mL) and extracted with EtOAc (20 mL×3). The organic layers were combined and washed with brine, dried over anhydrous Na₂SO₄ and concentrated under reduced pressure. The residue was purified by pre-TLC to give 6-(4-(cyclopropylcarbamoyl)phenoxy)-5-nitro-2,3-dihydro-1H-inden-1-yl di(aziridin-1-yl)phosphinat (45 mg, 93 μmol, 45%) as a colorless oil. ¹H NMR (400 MHz, CDCl₃) δ 7.77 (s, 1H), 7.67 (d, J=8.6 Hz, 2H), 7.20 (s, 1H), 6.93 (d, J=8.6 Hz, 2H), 6.21 (s, 1H), 5.91-5.81 (m, 1H), 3.13-3.03 (m, 1H), 2.92-2.77 (m, 2H), 2.63-2.50 (m, 1H), 2.30-2.21 (m, 1H), 2.15-1.99 (m, 8H), 0.87-0.72 (m, 4H). ³¹P NMR (162 MHz, CDCl₃) δ 29.98 (s). LC-MS (ESI): m/z 485.2 [M+H]⁺.

(R)-6-(4-(cyclopropylcarbamoyl)phenoxy)-5-nitro-2,3-dihydro-1H-inden-1-yl di(aziridin-1-yl)phosphinate & (S)-6-(4-(cyclopropylcarbamoyl)phenoxy)-5-nitro-2,3-dihydro-1H-inden-1-yl di(aziridin-1-yl)phosphinate

6-(4-(cyclopropylcarbamoyl)phenoxy)-5-nitro-2,3-dihydro-1H-inden-1-yl di(aziridin-1-yl)phosphinat (60 mg, 0.12 mmol) was purified by Chiral SFC to give:

Isomer 1 (173),20.7 mg, 42.8 μmol, 36%, Retention time: 3.370 min, 97% ee. LC-MS (ESI): m/z 485.1 [M+H]⁺; ¹H NMR (400 MHz, CDCl₃) δ 7.84 (s, 1H), 7.74 (d, J=8.8 Hz, 2H), 7.27 (s, 1H), 7.03-6.97 (m, 2H), 6.27 (s, 1H), 5.97-5.89 (m, 1H), 3.22-3.11 (m, 1H), 2.98-2.85 (m, 2H), 2.70-2.57 (m, 1H), 2.38-2.25 (m, 1H), 2.23-2.05 (m, 8H), 0.91-0.84 (m, 2H), 0.65-0.58 (m, 2H). ³¹P NMR (162 MHz, CDCl₃) δ 29.97 (s).

Isomer 2 (172), 13.4 mg, 27.7 μmol, 23%, Retention time: 4.064 min, 97% ee. LC-MS (ESI): m/z 485.1 [M+H]⁺; ¹H NMR (400 MHz, CDCl₃) δ 7.84 (s, 1H), 7.73 (d, J=8.7 Hz, 2H), 7.26 (s, 1H), 7.00 (t, J=8.0 Hz, 2H), 6.31 (s, 1H), 5.96-5.91 (m, 1H), 3.24-3.11 (m, 1H), 2.99-2.86 (m, 2H), 2.71-2.57 (m, 1H), 2.34-2.30 (m, 1H), 2.25-2.04 (m, 8H), 0.89-0.84 (m, 2H), 0.69-0.57 (m, 2H). ³¹P NMR (162 MHz, CDCl₃) & 29.97 (s).

Analytical method: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

SFC Method: Instrument: SHIMADZU PREP SOLUTION SFC, Column: ChiralPak C-IG, 250×30 mm I.D., 5 μm, Mobile phase: A for CO₂ and B for MEOH+0.1% NH₃H₂O, Gradient: B 40%. Flow rate: 70 mL/min, Back pressure: 100 bar, Column temperature: 35° C., Wavelength: 220 nm. Cycle-time: 4 min, Eluted time: 2 h.

### Illustration 26. Synthesis of (R)-5-nitro-6-(4-(pyridin-2-yl)phenoxy)-2,3-dihydro-1H-inden-1-yl di(aziridin-1-yl)phosphinate & (S)-5-nitro-6-(4-(pyridin-2-yl)phenoxy)-2,3-dihydro-1H-inden-1-yl di(aziridin-1-yl)phosphinate (188&189)

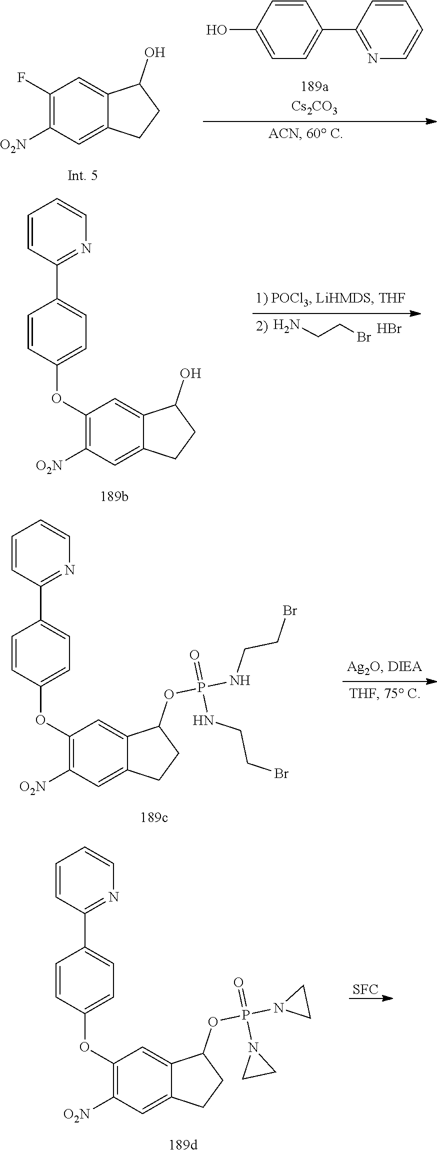

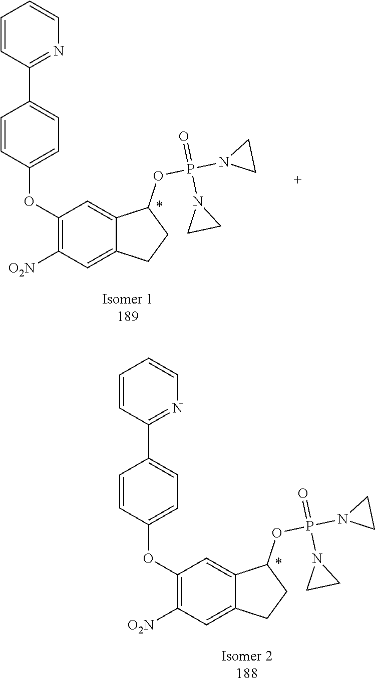 [SMILES: O=[N+]([O-])c1cc2c(cc1Oc1ccc(-c3ccccn3)cc1)C(OP(=O)(N1CC1)N1CC1)CC2 ; O=[N+]([O-])c1cc2c(cc1Oc1ccc(-c3ccccn3)cc1)C(OP(=O)(N1CC1)N1CC1)CC2]

5-nitro-6-(4-(pyridin-2-yl)phenoxy)-2,3-dihydro-1H-inden-1-ol

To a solution of 6-fluoro-5-nitro-2,3-dihydro-1H-inden-1-ol (400 mg, 2.03 mmol, 1.0 eq.) and 4-(pyridin-2-yl)phenol (486 mg, 2.84 mmol, 1.4 eq.) in ACN (5 mL) was added Cs₂CO₃ (1322 mg, 4.06 mmol, 2.0 eq.). The reaction mixture was stirred at 60° C. for 2 hrs. After completion, the reaction mixture was concentrated under reduced pressure. The residue was purified by Biotage® C18 column chromatography to give 5-nitro-6-(4-(pyridin-2-yl)phenoxy)-2,3-dihydro-1H-inden-1-ol (320 mg, 0.83 mmol, 41%) as a white solid. LCMS (ESI): m/z 349.1 [M+H]⁺.

5-nitro-6-(4-(pyridin-2-yl)phenoxy)-2,3-dihydro-1H-inden-1-yl di((2-bromoethyl)amino)phosphinate

To a stirred solution of 5-nitro-6-(4-(pyridin-2-yl)phenoxy)-2,3-dihydro-1H-inden-1-ol (50 mg, 0.14 mmol, 1.0 eq.) in THF (10 mL) was added LiHMDS (0.22 mL, 0.22 mmol, 1.6 eq.) dropwise at −65° C. and the resulting solution was stirred for 15 min under N₂. POCl₃ (0.02 mL, 0.23 mmol, 1.6 eq.) in THF (1 mL) was added to the above mixture at −65° C. and the resulting solution was stirred for 15 min under N₂. 2-bromoethan-1-amine hydrobromide (207 mg, 1.01 mmol, 7.0 eq.) and TEA (0.24 mL, 1.72 mmol, 12.3 eq.) were added to the above mixture at −65° C. and the resulting solution was stirred for 30 min under N₂. Then the stirred solution was warmed up to room temperature and stirred for 0.5 hrs. After completion, the reaction mixture was quenched with NH₄Cl solution (5 mL, sat., aq.) and extracted with EtOAc (10 mL×2). The organic layers were combined and washed with brine (5 mL), dried over anhydrous Na₂SO₄ and concentrated under reduced pressure. The residue was purified by Biotage® C18 column chromatography to afford 5-nitro-6-(4-(pyridin-2-yl)phenoxy)-2,3-dihydro-1H-inden-1-yl di((2-bromoethyl)amino)phosphinate (40 mg, 62 μmol, 44%) as a yellow oil. LCMS (ESI): m/z 641.3 [M+H]⁺.

5-nitro-6-(4-(pyridin-2-yl)phenoxy)-2,3-dihydro-1H-inden-1-yl di(aziridin-1-yl)phosphinate

To a solution of 5-nitro-6-(4-(pyridin-2-yl)phenoxy)-2,3-dihydro-1H-inden-1-yl di((2-bromoethyl)amino)phosphinate (150 mg, 0.23 mmol, 1.0 eq.) in THF (15 mL) were added Ag₂O (543 mg, 2.34 mmol, 10.0 eq.) and DIEA (302 mg, 2.34 mmol, 10.0 eq.). The resulting solution was stirred at 75° C. for 18 hrs under N₂. After completion, the reaction mixture was filtered and the filtrate was concentrated under reduced pressure. The residue was purified by flash column chromatography on silica gel to afford 5-nitro-6-(4-(pyridin-2-yl)phenoxy)-2,3-dihydro-1H-inden-1-yl di(aziridin-1-yl)phosphinate (100 mg, 0.20 mmol, 85%) as a yellow solid in a stereo isomeric mixture form. ¹H NMR (400 MHz, CDCl₃) δ 8.62-8.58 (m, 1H), 7.97-7.90 (m, 2H), 7.76 (s, 1H), 7.71-7.65 (m, 1H), 7.63 (d, J=7.9 Hz, 1H), 7.19-7.13 (m, 2H), 7.07-7.03 (m, 2H), 5.94-5.79 (m, 1H), 3.14-3.00 (m, 1H), 2.90-2.77 (m, 1H), 2.59-2.46 (m, 1H), 2.29-2.19 (m, 1H), 2.11-1.97 (m, 8H). ³¹P NMR (162 MHz, CDCl₃) δ 29.94 (s). LC-MS (ESI): m/z 479.1 [M+H]⁺.

(R)-5-nitro-6-(4-(pyridin-2-yl)phenoxy)-2,3-dihydro-1H-inden-1-yl di(aziridin-1-yl)phosphinate & (S)-5-nitro-6-(4-(pyridin-2-yl)phenoxy)-2,3-dihydro-1H-inden-1-yl di(aziridin-1-yl)phosphinate

5-nitro-6-(4-(pyridin-2-yl)phenoxy)-2,3-dihydro-1H-inden-1-yl di(aziridin-1-yl)phosphinate (80 mg, 0.17 mmol) was further separated by Chiral SFC to give:

Isomer 1 (189), 35 mg, 44%; Retention time: 3.960 min, >99% ee. LC-MS (ESI): m/z 479.1 [M+H]⁺; ¹H NMR (400 MHz, CDCl₃) δ 8.67 (d, J=4.7 Hz, 1H), 8.01 (d, J=8.8 Hz, 2H), 7.83 (s, 1H), 7.78-7.72 (m, 1H), 7.70 (d, J=7.9 Hz, 1H), 7.26-7.20 (m, 2H), 7.12 (d, J=8.8 Hz, 2H), 5.97-5.85 (m, 1H), 3.18-3.10 (m, 1H), 2.96-2.86 (m, 1H), 2.66-2.55 (m, 1H), 2.35-2.24 (m, 1H), 2.16-2.00 (m, 8H). ³¹P NMR (162 MHz, CDCl₃) δ 29.94 (s).

Isomer 2 (188), 33 mg, 41%; Retention time: 5.393 min, >99% ee. LC-MS (ESI): m/z 479.1 [M+H]⁺; ¹H NMR (400 MHz, CDCl₃) δ 8.67 (d, J=4.4 Hz, 1H), 8.01 (d, J=8.7 Hz, 2H), 7.83 (s, 1H), 7.79-7.72 (m, 1H), 7.69 (d, J=7.9 Hz, 1H), 7.26-7.19 (m, 2H), 7.12 (d, J=8.7 Hz, 2H), 6.03-5.81 (m, 1H), 3.21-3.08 (m, 1H), 2.95-2.81 (m, 1H), 2.68-2.55 (m, 1H), 2.34-2.26 (m, 1H), 2.17-2.03 (m, 8H). ³¹P NMR (162 MHz, CDCl₃) δ 29.94 (s).

Analytical method: Column: ChiralCel OJ, 250×4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C.

SFC Method: Instrument: Waters Thar 80 preparative SFC, Column: ChiralPak OJ, 250×21.2 mm I.D., 5 μm, Mobile phase: A for CO₂ and B for MEOH+0.1% NH₃H₂O, Gradient: B 30%, Flow rate: 40 mL/min, Back pressure: 100 bar, Column temperature: 35° C., Wavelength: 220 nm, Cycle-time: 15 min, Eluted time: 2 H.

### Illustration 27. Synthesis of 6-((2-methyl-3-oxoisoindolin-5-yl)oxy)-7-nitrochroman-4-yl di(aziridin-1-yl)phosphinate (199) (General Procedure7, 6,6 core 3-method A)

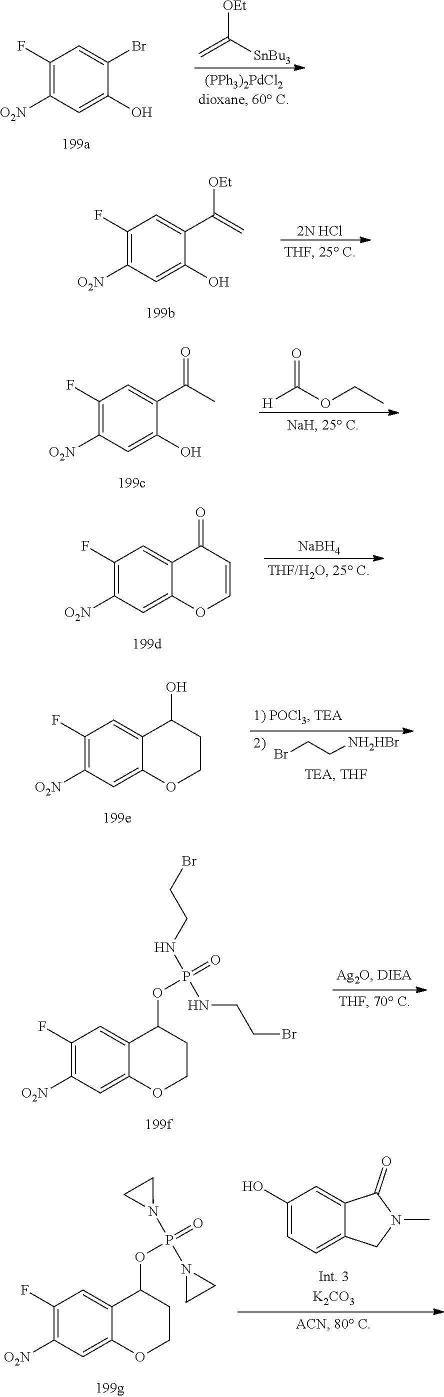

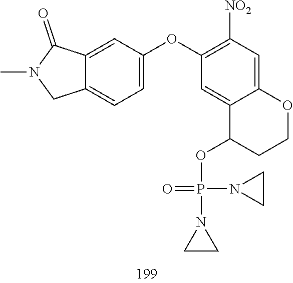 [SMILES: CN1Cc2ccc(Oc3cc4c(cc3[N+](=O)[O-])OCCC4OP(=O)(N3CC3)N3CC3)cc2C1=O]

2-(1-ethoxyvinyl)-4-fluoro-5-nitrophenol

To a solution of 2-bromo-4-fluoro-5-nitrophenol (10.0 g, 42.37 mmol, 1.0 eq.) and tributyl(1-ethoxyethenyl) stannane (18.36 g, 50.85 mmol, 1.2 eq.) in dioxane (150 mL) was added (PPh₃)₂PdCl₂ (1.09 g, 1.40 mmol, 0.03 eq.) at 25° C. The mixture was evaporated and backfilled with nitrogen for three times, then stirred at 60° C. for 24 hrs under nitrogen atmosphere. After completion, the reaction mixture was cooled to 25° C., then the reaction mixture was quenched by addition of sat. KF (50 mL) at 25° C. and stirred at 25° C. for 1 hr. The solid was removed by filtration. The filtrate was concentrated under reduced pressure to give 2-(1-ethoxyethenyl)-4-fluoro-5-nitrophenol (17 g, quant) as a brown oil, which was used in next step directly without further purification. LC-MS (ESI): m/z 228 [M+H]⁺.

1-(5-fluoro-2-hydroxy-4-nitrophenyl) ethan-1-one

To a solution of 2-(1-ethoxyethenyl)-4-fluoro-5-nitrophenol (8.50 g, 37.41 mmol, 1.0 eq.) in THF (50 mL) was added drop-wisely HCl aqueous solution (50 mL, 2 N,) at 0-5° C. The reaction mixture was stirred at 25° C. for 2 hrs. After completion, the reaction mixture was poured into water (100 mL) at 25° C. and extracted with EtOAc (50 mL×2). The combined organic layers were washed with brine (100 mL), dried over anhydrous Na₂SO₄ and concentrated under reduced pressure. The residue was purified by flash column chromatography on silica gel to give 1-(5-fluoro-2-hydroxy-4-nitrophenyl) ethan-1-one (3.20 g, 16.1 mmol, 43%) as a yellow solid. LC-MS (ESI): m/z 200 [M+H]⁺.

6-fluoro-7-nitro-4H-chromen-4-one

To a solution of 1-(5-fluoro-2-hydroxy-4-nitrophenyl) ethan-1-one (900 mg, 4.52 mmol, 1.0 eq.) in ethyl formate (18 mL) was added NaH (1.08 g, 27.12 mmol, 6.0 eq.) in portions at 0-5° C. The mixture was warmed up at 25° C. and stirred for 2 hrs. After completion, MeOH (1.8 mL) and HCl (5.4 mL) was added to the mixture at 10° C. The mixture was stirred at 25° C. for 2 hrs, then poured into water (100 mL) and extracted with EtOAc (50 mL×2). The combined organic layers were washed with H₂O (50 mL), dried over anhydrous Na₂SO₄, filtered and concentrated under reduced pressure. The residue was purified by flash column chromatography on silica gel to give 6-fluoro-7-nitro-4H-chromen-4-one (600 mg, 2.87 mmol, 63%) as a yellow solid. LC-MS (ESI): m/z 210 [M+H]⁺.

6-fluoro-7-nitrochroman-4-ol

To a solution of 6-fluoro-7-nitro-4H-chromen-4-one (600 mg, 2.87 mmol, 1.0 eq.) in THF/H₂O (6 mL/0.6 mL) was added NaBH₄ (218 mg, 5.74 mmol, 2.0 eq.) at 0° C. The reaction mixture was warmed up at 25° C. and stirred for 1 hr. After completion, the reaction mixture was quenched by addition of HCl (5 mL, aq. 1N) and extracted with DCM (10 mL×2). The combined organic phases were washed with brine (10 mL), dried over anhydrous Na₂SO₄, filtered and concentrated under reduced pressure. The residue was purified by flash column chromatography on silica gel to give 6-fluoro-7-nitrochroman-4-ol (360 mg, 1.69 mmol, 59%) as a yellow solid. ¹H NMR (400 MHz, DMSO-d₆) δ 7.51-7.43 (m, 2H), 5.82 (d, J=5.5 Hz, 1H), 4.70 (dd, J=11.5, 5.5 Hz, 1H), 4.38-4.19 (m, 2H), 2.12-1.83 (m, 2H)

6-fluoro-7-nitrochroman-4-yl di((2-bromoethyl)amino)phosphinate

To a solution of POCl₃ (0.31 mL, 3.28 mmol, 2.0 eq.) in a mixture of DCM (10 mL) and THF (5 mL) was added drop-wisely TEA (1.83 mL, 13.14 mmol, 8.0 eq.) followed with a solution of 6-fluoro-7-nitrochroman-4-ol (350 mg, 1.64 mmol, 1.0 eq.) in THF (5 mL) at −40° C. under nitrogen atmosphere. After addition, the mixture was stirred at −40° C. for 2 hrs. 2-bromoethan-1-amine hydrobromide (2.35 g, 11.49 mmol, 7.0 eq.) and TEA (1.83 mL, 13.14 mmol, 8.0 eq.) were added into the mixture. After addition, the reaction mixture was stirred at −40° C. for 1 hr. After completion, the reaction mixture was warmed up to 0° C., quenched with sat. NH₄Cl solution (10 mL) and extracted with DCM (10 mL×2). The combined organic phases were washed with brine (10 mL), dried over anhydrous Na₂SO₄, filtered and concentrated under reduced pressure. The residue was purified by Biotage® C₁₈ column chromatography to give 6-fluoro-7-nitrochroman-4-yl di((2-bromoethyl)amino)phosphinate (500 mg, 0.99 mmol, 60%) as a yellow oil. LC-MS (ESI): m/z 504 [M+H]⁺.

6-fluoro-7-nitrochroman-4-yl di(aziridin-1-yl)phosphinate

To a solution of 6-fluoro-7-nitrochroman-4-yl di((2-bromoethyl)amino)phosphinate (500 mg, 0.99 mmol, 1.0 eq.) in THF (10 mL) were added DIEA (1 mL, 6.05 mmol, 6.1 eq.) and Ag₂O (0.21 mL, 6.47 mmol, 6.5 eq.) at 25° C. The reaction mixture was heated to 70° C. and stirred at 70° C. for 12 hrs under nitrogen atmosphere. After completion, the reaction mixture was cooled to 25° C. and the suspension was filtered through a pad of celite. The filter cake was washed with DCM (100 mL). The combined filtrates was concentrated under reduced pressure to give 6-fluoro-7-nitrochroman-4-yl di(aziridin-1-yl)phosphinate (330 mg, 0.96 mmol, 97%) as a yellow solid. LC-MS (ESI): m/z 344 [M+H]⁺.

6-((2-methyl-3-oxoisoindolin-5-yl)oxy)-7-nitrochroman-4-yl di(aziridin-1-yl)phosphinate

To a solution of 6-fluoro-7-nitrochroman-4-yl di(aziridin-1-yl)phosphinate (70 mg, 0.20 mmol, 1.0 eq.) and 6-hydroxy-2-methyl-2,3-dihydro-1H-isoindol-1-one (39.93 mg, 0.24 mmol, 1.2 eq.) in ACN (4 mL) was added K₂CO₃ (140.97 mg, 1.02 mmol, 5.0 eq) at 25° C. The reaction mixture was heated to 80° C. and stirred for 4 hrs under nitrogen atmosphere. After completion, the reaction mixture was poured into ice-water (5 mL), then extracted with EtOAc (5 mL×2). The combined organic layers were washed with brine (5 mL), dried over anhydrous Na₂SO₄, filtered and concentrated under reduced pressure. The residue was purified by RP-prep HPLC to give 6-((2-methyl-3-oxoisoindolin-5-yl)oxy)-7-nitrochroman-4-yl di(aziridin-1-yl)phosphinate (5.5 mg, 11 μmol, 5%) as a white solid. ¹H NMR (400 MHz, CDCl₃) δ 7.49 (d, J=5.9 Hz, 1H), 7.41 (d, J=8.2 Hz, 1H), 7.34 (s, 1H), 7.24 (d, J=2.4 Hz, 1H), 7.19 (d, J=2.3 Hz, 1H), 5.65-5.45 (m, 1H), 4.42-4.30 (m, 4H), 3.18 (s, 3H), 2.34-2.26 (m, 2H), 2.25-2.02 (m, 8H). ³¹P NMR (162 MHz, CDCl₃) δ 29.75 (s). LC-MS (ESI): m/z 487.1 [M+H]⁺.

### Illustration 28. Synthesis of (S)-6-((2-methyl-3-oxoisoindolin-5-yl)oxy)-7-nitrochroman-4-yl di(aziridin-1-yl)phosphinate & (R)-6-((2-methyl-3-oxoisoindolin-5-yl)oxy)-7-nitrochroman-4-yl di(aziridin-1-yl)phosphinate (201&202) (General Procedure 7, 6,6 core 3-method B)

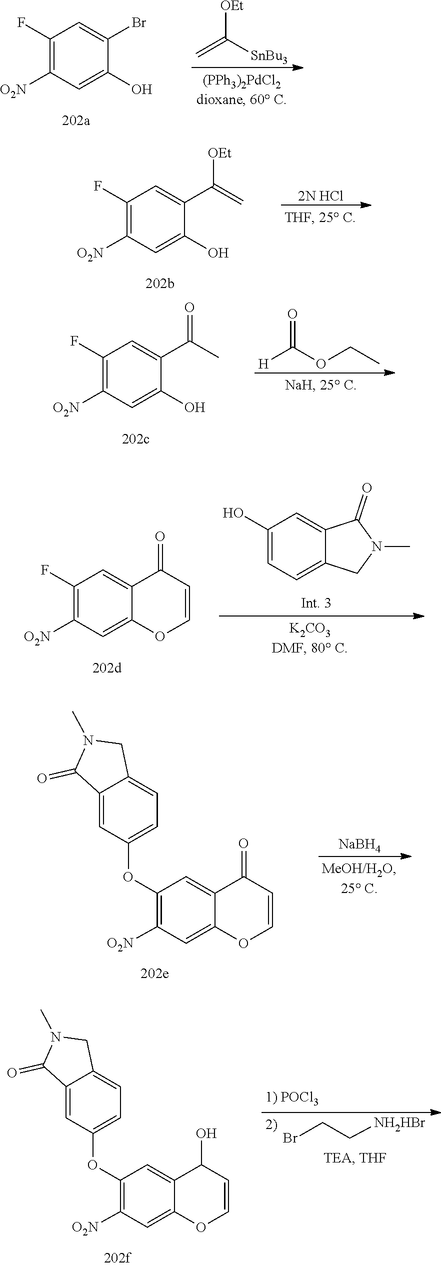

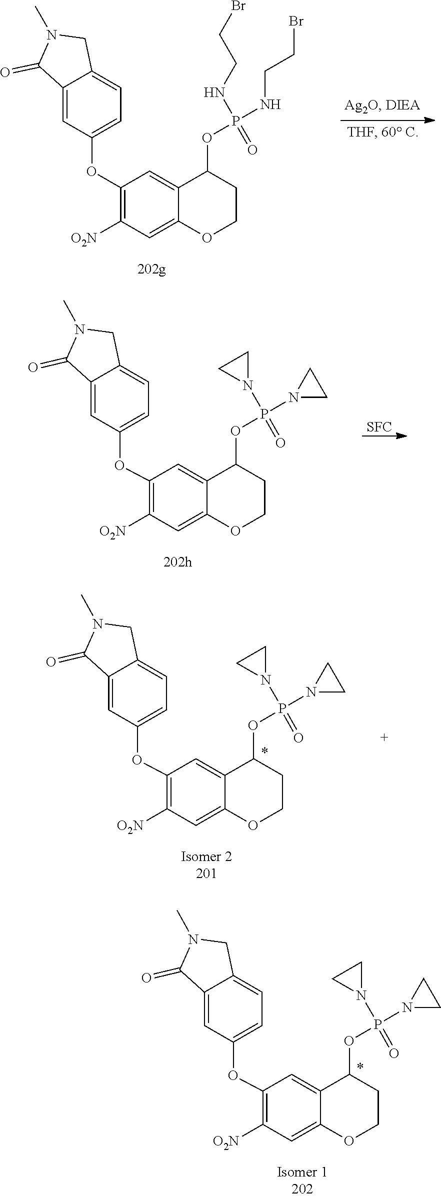

2-(1-ethoxyvinyl)-4-fluoro-5-nitrophenol

To a solution of 2-bromo-4-fluoro-5-nitrophenol (10.0 g, 42.4 mmol, 1.0 eq.) and tributyl(1-ethoxyethenyl) stannane (18.4 g, 50.9 mmol, 1.2 eq.) in dioxane (150 mL) was added (PPh₃)₂PdCl₂ (1.09 g, 1.40 mmol, 0.03 eq.) at 25° C. The mixture was evaporated and backfilled with nitrogen for three times, then stirred at 60° C. for 24 hrs under nitrogen atmosphere. After completion, the reaction mixture was cooled to 25° C., then the reaction mixture was quenched by addition of sat. KF (50 mL) and stirred at 25° C. for 1 hr. The solid was removed by filtration. The filtrate was concentrated under reduced pressure to give 2-(1-ethoxyethenyl)-4-fluoro-5-nitrophenol (17 g, quant) as a brown oil, which was used in next step directly without further purification. LC-MS (ESI): m/z 228 [M+H]⁺.

1-(5-fluoro-2-hydroxy-4-nitrophenyl) ethan-1-one

To a solution of 2-(1-ethoxyethenyl)-4-fluoro-5-nitrophenol (8.50 g, 37.4 mmol, 1.0 eq.) in THF (50 mL) was added drop-wisely HCl aqueous solution (50 mL, 2 N,) at 0-5° C. The reaction mixture was warmed up to 25° C. and stirred for 2 hrs. After completion, the reaction mixture was poured into water (100 mL) and extracted with EtOAc (50 mL×2). The combined organic layers were washed with brine (100 mL), dried over anhydrous Na₂SO₄ and concentrated under reduced pressure. The residue was purified by flash column chromatography on silica gel to give 1-(5-fluoro-2-hydroxy-4-nitrophenyl) ethan-1-one (3.20 g, 16.07 mmol, 43%) as a yellow solid. LC-MS (ESI): m/z 200 [M+H]⁺.

6-fluoro-7-nitro-4H-chromen-4-one

To a solution of 1-(5-fluoro-2-hydroxy-4-nitrophenyl) ethan-1-one (900 mg, 4.52 mmol, 1.0 eq.) in ethyl formate (18 mL) was added NaH (1.08 g, 27.12 mmol, 6.0 eq.) in portions at 0-5° C. The mixture was warmed up to 25° C. and stirred for 2 hrs. After completion, MeOH (1.8 mL) and HCl (5.4 mL) was added to the mixture at 10° C. The mixture was stirred at 25° C. for 2 hrs, then poured into water (100 mL) and extracted with EtOAc (50 mL×2). The combined organic layers were washed with H₂O (50 mL), dried over anhydrous Na₂SO₄, filtered and concentrated under reduced pressure. The residue was purified by flash column chromatography on silica gel to give 6-fluoro-7-nitro-4H-chromen-4-one (600 mg, 2.87 mmol, 63%) as a yellow solid. LC-MS (ESI): m/z 210 [M+H]⁺.

2-methyl-6-((7-nitro-4-oxo-4H-chromen-6-yl)oxy) isoindolin-1-one

To a solution of 6-fluoro-7-nitro-4H-chromen-4-one (500 mg, 2.39 mmol, 1.0 eq.) and 6-hydroxy-2-methyl-2,3-dihydro-1H-isoindol-1-one (468 mg, 2.87 mmol, 1.2 eq.) in DMF (10 mL) was added K₂CO₃ (991 mg, 7.17 mmol, 3.0 eq) and the reaction mixture was stirred at 40° C. for 18 hrs under nitrogen atmosphere. After completion, the reaction mixture was triturated with water and filtered. The filter cake was washed with water and then dried under vacuum to afford 2-methyl-6-((7-nitro-4-oxo-4H-chromen-6-yl)oxy) isoindolin-1-one (690 mg, 1.96 mmol, 82%) as a solid. LC-MS (ESI): m/z 353 [M+H]⁺.

6-((4-hydroxy-7-nitrochroman-6-yl)oxy)-2-methylisoindolin-1-one

To a solution of 2-methyl-6-((7-nitro-4-oxo-4H-chromen-6-yl)oxy) isoindolin-1-one (690 mg, 1.96 mmol, 1.0 eq.) in MeOH/H₂O (30 mL/3 mL) was added NaBH₄ (132 mg, 3.92 mmol, 2.0 eq.) at 0° C. The reaction mixture was warmed up to 25° C. and stirred for 1 hr. After completion, the reaction mixture was quenched by addition of HCl (6 mL, aq. 1N) and extracted with EtOAc (20 mL×2). The organic layers were combined and washed with brine (10 mL), dried over anhydrous Na₂SO₄ and concentrated under reduced pressure. The residue was purified by flash column chromatography on silica gel to give 6-((4-hydroxy-7-nitrochroman-6-yl)oxy)-2-methylisoindolin-1-one (340 mg, 0.95 mmol, 49%) as a yellow solid. LC-MS (ESI): m/z 357.0 [M+H]⁺.

6-((2-methyl-3-oxoisoindolin-5-yl)oxy)-7-nitrochroman-4-yl di((2-bromoethyl)amino)phosphinate

To a solution of 6-((4-hydroxy-7-nitrochroman-6-yl)oxy)-2-methylisoindolin-1-one (250 mg, 0.70 mmol, 1.0 eq.) in THF (20 mL) was added LiHMDs (1.4 mL, 1.40 mmol, 2.0 eq.) at −40° C. The mixture was stirred at −40° C. for 0.5 hrs. POCl₃ (215 mg, 1.40 mmol, 2.0 eq.) was added and the reaction mixture was stirred at −40° C. for 20 min. 2-bromoethan-1-amine hydrobromide (862 mg, 4.21 mmol, 6.0 eq.) and TEA (850 mg, 8.42 mmol, 12.0 eq.) was added into the mixture. Then the mixture was warmed up to room temperature and stirred for 40 min. After completion, the reaction mixture was quenched with saturated NH₄Cl aqueous solution (10 mL) and extracted with EtOAc (10 mL). The organic layers was combined and washed brine (10 mL), dried over anhydrous Na₂SO₄ and concentrated under reduced pressure. The residue was purified by Biotage® C18 column chromatography to give 6-((2-methyl-3-oxoisoindolin-5-yl)oxy)-7-nitrochroman-4-yl di((2-bromoethyl)amino)phosphinate (50 mg, 77 μmol, 11%) as a yellow oil.

(S)-6-((2-methyl-3-oxoisoindolin-5-yl)oxy)-7-nitrochroman-4-yl di(aziridin-1-yl)phosphinate & (R)-6-((2-methyl-3-oxoisoindolin-5-yl)oxy)-7-nitrochroman-4-yl di(aziridin-1-yl)phosphinate

To a solution of 6-((2-methyl-3-oxoisoindolin-5-yl)oxy)-7-nitrochroman-4-yl di((2-bromoethyl)amino)phosphinate (50 mg, 77 μmol, 1.0 eq.) in THF (5 mL) were added DIEA (60 mg, 0.46 mmol, 6.0 eq.) and Ag₂O (107 mg, 0.46 mmol, 6.0 eq.). The reaction mixture was stirred at 60° C. for 12 hrs under nitrogen atmosphere. After completion, the reaction mixture was cooled to 25° C. and the suspension was filtered through a pad of celite. The filter cake was washed with DCM (10 mL). The combined filtrates was concentrated under reduced pressure and purified by flash column chromatography on silica gel to give 6-((2-methyl-3-oxoisoindolin-5-yl)oxy)-7-nitrochroman-4-yl di(aziridin-1-yl)phosphinate in a stereo isomeric mixture form, which was further separated by Chiral SFC to give:

Isomer 1 (202), 2.4 mg, 12%, Retention time: 5.499 min, 99.5% ee. LC-MS (ESI): m/z 487.2 [M+H]⁺; ¹H NMR (400 MHz, CDCl₃) δ 7.52-7.46 (m, 1H), 7.44-7.38 (m, 1H), 7.34 (s, 1H), 7.27-7.24 (m, 1H), 7.21-7.18 (m, 1H), 5.61-5.52 (m, 1H), 4.45-4.28 (m, 4H), 3.19 (s, 3H), 2.37-2.26 (m, 2H), 2.23-2.03 (m, 8H). ³¹P NMR (162 MHz, CDCl₃) δ 29.76 (s).

Isomer 2 (201), 2.8 mg, 14%, Retention time: 5.967 min, 85.4% ee. LC-MS (ESI): m/z 487.2 [M+H]⁺; ¹H NMR (400 MHz, CDCl₃) δ 7.52-7.46 (m, 1H), 7.44-7.32 (m, 2H), 7.24-7.17 (m, 2H), 5.57-5.37 (m, 1H), 4.43-4.30 (m, 4H), 3.18 (s, 3H), 2.35-1.95 (m, 10H). ³¹P NMR (162 MHz, CDCl₃) δ 29.74 (s).

Analytical method: Column: ChiralCel OD, 250×4.6 mm I.D., 5 μm, Mobile phase: A for CO₂ and B for EtOH (0.05% DEA), Gradient: 8 min @ B 30%, Flow rate: 2.0 mL/min, Back pressure: 100 bar, Column temperature: 35° C.

SFC Method: Instrument: Waters Thar 80 preparative SFC, Column: ChiralCel OD, 250×21.2 mm I.D., 5 μm, Mobile phase: A for CO₂ and B for ETOH+0.1% NH₃H₂O, Gradient: B 25%, Flow rate: 40 mL/min, Back pressure: 100 bar, Column temperature: 35° C., Wavelength: 220 nm, Cycle-time: 15 min, Eluted time: 2.0 H.

### Illustration 29. Synthesis of (S)-6-((2-cyclopropyl-3-oxoisoindolin-5-yl)oxy)-7-nitrochroman-4-yl di(aziridin-1-yl)phosphinate & (R)-6-((2-cyclopropyl-3-oxoisoindolin-5-yl)oxy)-7-nitrochroman-4-yl di(aziridin-1-yl)phosphinate (203&204)

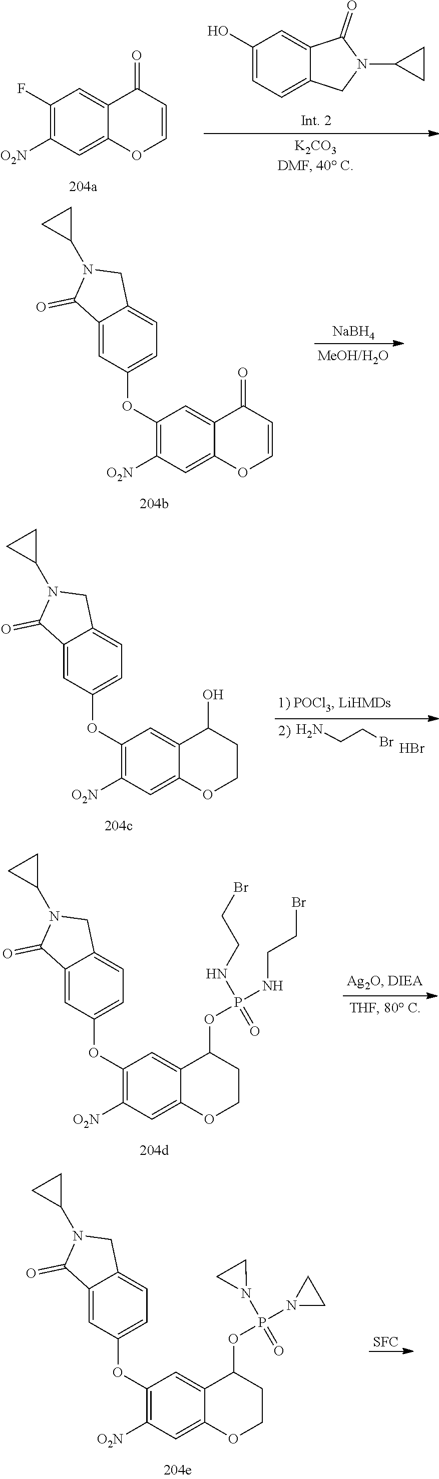

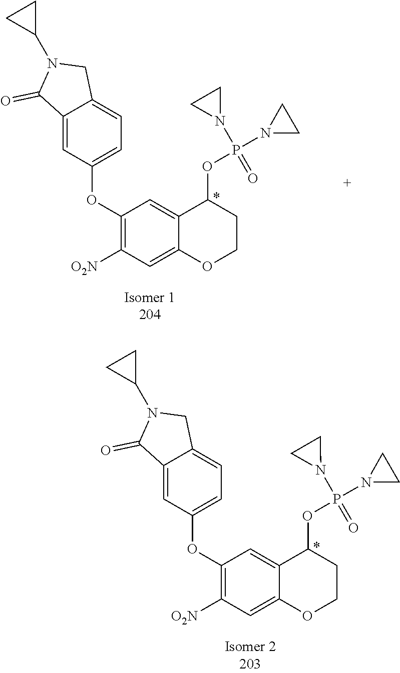 [SMILES: O=C1c2cc(Oc3cc4c(cc3[N+](=O)[O-])OCCC4OP(=O)(N3CC3)N3CC3)ccc2CN1C1CC1 ; O=C1c2cc(Oc3cc4c(cc3[N+](=O)[O-])OCCC4OP(=O)(N3CC3)N3CC3)ccc2CN1C1CC1]

2-cyclopropyl-6-((7-nitro-4-oxo-4H-chromen-6-yl)oxy) isoindolin-1-one

To a solution of 6-fluoro-7-nitro-4H-chromen-4-one (500 mg, 2.39 mmol, 1.0 eq.) and 2-cyclopropyl-6-hydroxyisoindolin-1-one (543 mg, 2.87 mmol, 1.2 eq.) in DMF (10 mL) was added K₂CO₃ (991 mg, 7.17 mmol, 3.0 eq) and the reaction mixture was stirred at 40° C. for 18 hrs under nitrogen atmosphere. After completion, the reaction mixture was triturated with water and filtered. The filter cake was washed with water and then dried under vacuum to afford 2-cyclopropyl-6-((7-nitro-4-oxo-4H-chromen-6-yl)oxy) isoindolin-1-one (880 mg, 2.33 mmol, 97%) as a solid, which was used in next step directly without purification. LC-MS (ESI): m/z 379 [M+H]⁺.

2-cyclopropyl-6-((4-hydroxy-7-nitrochroman-6-yl)oxy)isoindolin-1-one

To a solution of 2-cyclopropyl-6-((7-nitro-4-oxo-4H-chromen-6-yl)oxy) isoindolin-1-one (830 mg, 2.19 mmol, 1.0 eq.) in MeOH/H₂O (30 mL/3 mL) was added NaBH₄ (148 mg, 4.39 mmol, 2.0 eq.) at 0° C. The reaction mixture was warmed up to 25° C. and stirred for 1 hr. After completion, the reaction mixture was quenched by addition of HCl (6 mL, aq. 1N) and extracted with EtOAc (20 mL×2). The organic layers were combined and washed with brine (10 mL), dried over anhydrous Na₂SO₄ and concentrated under reduced pressure. The residue was purified by flash column chromatography on silica gel to give 2-cyclopropyl-6-((4-hydroxy-7-nitrochroman-6-yl)oxy) isoindolin-1-one (240 mg, 0.63 mmol, 29%) as a yellow solid. LC-MS (ESI): m/z 383 [M+H]⁺.

6-((2-cyclopropyl-3-oxoisoindolin-5-yl)oxy)-7-nitrochroman-4-yl di((2-bromoethyl)amino)phosphinate

To a solution of 2-cyclopropyl-6-((4-hydroxy-7-nitrochroman-6-yl)oxy)isoindolin-1-one (210 mg, 0.55 mmol, 1.0 eq.) in THF (20 mL) was added LiHMDS (1.1 mL, 1.10 mmol, 2.0 eq.) at −60° C. The mixture was stirred at −60° C. for 15 min. POCl₃ (0.06 mg, 0.61 mmol, 1.1 eq.) was added and the reaction mixture was stirred at −60° C. for 15 min. 2-bromoethan-1-amine hydrobromide (675 mg, 3.30 mmol, 6.0 eq.) and TEA (667 mg, 6.60 mmol, 12.0 eq.) were added into the mixture. Then the mixture was warmed up to room temperature and stirred for 40 min. After completion, the reaction mixture was quenched with saturated NH₄Cl aqueous solution (10 mL) and extracted with EtOAc (10 mL). The organic layers was combined and washed brine (10 mL), dried over anhydrous Na₂SO₄ and concentrated under reduced pressure. The residue was purified by Biotage® C18 column chromatography to give 6-((2-cyclopropyl-3-oxoisoindolin-5-yl)oxy)-7-nitrochroman-4-yl di((2-bromoethyl)amino)phosphinate (50 mg, 74 μmol, 14%) as a yellow oil. LC-MS (ESI): m/z 673 [M+H]⁺.

6-((2-cyclopropyl-3-oxoisoindolin-5-yl)oxy)-7-nitrochroman-4-yl di(aziridin-1-yl)phosphinate

To a solution of 6-((2-cyclopropyl-3-oxoisoindolin-5-yl)oxy)-7-nitrochroman-4-yl di((2-bromoethyl)amino)phosphinate (50 mg, 74 μmol, 1.0 eq.) in THF (5 mL) were added DIEA (57 mg, 0.45 mmol, 6.0 eq.) and Ag₂O (381 mg, 1.65 mmol, 6.0 eq.). The reaction mixture was stirred at 60° C. for 12 hrs under nitrogen atmosphere. After completion, the reaction mixture was cooled to 25° C. and filtered. The filter cake was washed with DCM (10 mL). The combined filtrates was concentrated under reduced pressure and purified by Biotage® C18 column chromatography to give 6-((2-cyclopropyl-3-oxoisoindolin-5-yl)oxy)-7-nitrochroman-4-yl di(aziridin-1-yl)phosphinate (10 mg, 20 μmol, 26%) as a yellow oil in a stereo isomeric mixture form. LC-MS (ESI): m/z 513.2 [M+H]⁺.

(S)-6-((2-cyclopropyl-3-oxoisoindolin-5-yl)oxy)-7-nitrochroman-4-yl di(aziridin-1-yl)phosphinate & (R)-6-((2-cyclopropyl-3-oxoisoindolin-5-yl)oxy)-7-nitrochroman-4-yl di(aziridin-1-yl)phosphinate

6-((2-cyclopropyl-3-oxoisoindolin-5-yl)oxy)-7-nitrochroman-4-yl di(aziridin-1-yl)phosphinate (10 mg, 20 μmol) was further separated by Chiral SFC to give:

Isomer 1 (204), 2.0 mg, 20%, Retention time: 1.536 min, 99% ee. LC-MS (ESI): m/z 513.2 [M+H]⁺; ¹H NMR (400 MHz, CDCl₃) δ 7.49 (s, 1H), 7.38 (d, J=8.3 Hz, 1H), 7.32 (s, 1H), 7.26-7.23 (m, 1H), 7.18 (d, J=2.3 Hz, 1H), 5.60-5.49 (m, 1H), 4.43-4.31 (m, 2H), 4.29 (s, 2H), 2.96-2.87 (m, 1H), 2.36-2.25 (m, 2H), 2.22-1.97 (m, 8H), 0.94-0.81 (m, 4H). ³¹P NMR (162 MHz, CDCl₃) & 29.74 (s).

Isomer 2 (203), 2.0 mg, 20%, Retention time: 2.482 min, 99% ee. LC-MS (ESI): m/z 513.2 [M+H]⁺; ¹H NMR (400 MHz, CDCl₃) δ 7.49 (s, 1H), 7.38 (d, J=8.3 Hz, 1H), 7.32 (s, 1H), 7.26-7.22 (m, 1H), 7.18 (d, J=2.3 Hz, 1H), 5.58-5.51 (m, 1H), 4.42-4.30 (m, 2H), 4.29 (s, 2H), 2.95-2.89 (m, 1H), 2.35-2.27 (m, 2H), 2.22-2.05 (m, 8H), 1.00-0.71 (m, 4H). ³¹P NMR (162 MHz, CDCl₃) ¿ 29.74 (s).

Analytical method: Column: ChiralPak IH, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

SFC Method: Instrument: Waters Thar 80 preparative SFC, Column: ChiralPak IH, 250×21.2 mm I.D., 5 μm, Mobile phase: A for CO₂ and B for MEOH+0.1% NH₃H₂O, Gradient: B 40%, Flow rate: 40 mL/min, Back pressure: 100 bar, Column temperature: 35° C., Wavelength: 220 nm, Cycle-time: 8 min, Eluted time: 1.5 H.

### Illustration 30. Synthesis of 3-(3-(dimethylcarbamoyl)phenoxy)-4-nitrobenzyl bis(2-methylaziridin-1-yl)phosphinate (206) (General Procedure 4)

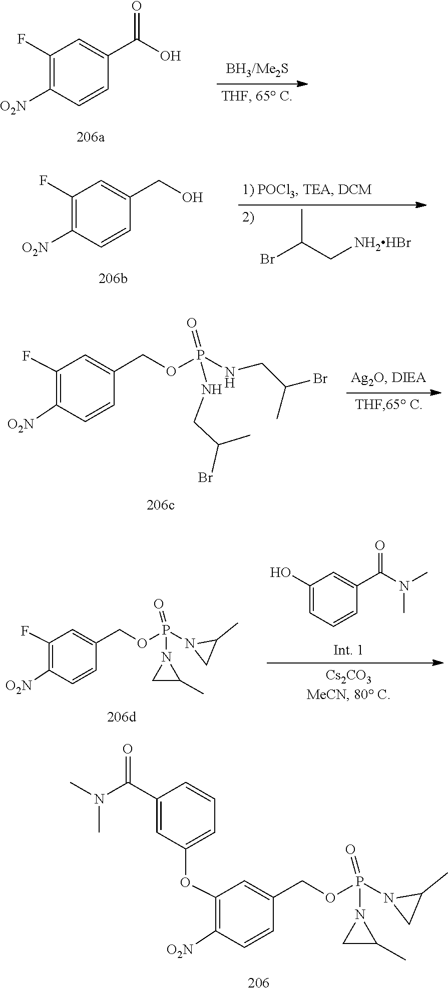

(3-fluoro-4-nitrophenyl)methaol

To a solution of 3-fluoro-4-nitrobenzoic acid (2.0 g, 10.80 mmol, 1.0 eq.) in THF (50 mL) at 0° C. was added borane-methyl sulfide complex (11.34 mL, 22.69 mmol, 2N, 2.1 eq.) drop-wisely under nitrogen. The mixture was stirred at 0° C. for 3 hrs, and then stirred at 65° C. for 18 hrs. After completion, the reaction mixture was cooled down to room temperature, then quenched with 1 N aq. HCl (20 mL). The resulting mixture was extracted with EtOAc (60 ml), and the organic layer was dried over anhydrous Na₂SO₄, then concentrated under reduced pressure to give (3-fluoro-4-nitrophenyl) methanol (1.8 g, 10.52 mmol, 97%) as a yellow solid, which was used in next step directly without further purification. LC-MS (ESI): m/z 172 [M+H]⁺. ¹HNMR (400 MHz, CDCl₃) δ 8.09-8.02 (m, 1H), 7.34 (d, J=11.7 Hz, 1H), 7.28-7.24 (m, 1H), 4.81 (s, 2H), 2.07 (s, 1H). 3-fluoro-4-nitrobenzyl di(2-bromopropan-1-yl amino)phosphinate

To a solution of POCl₃ (179.20 mg, 1.17 mmol, 2.0 eq.) in DCM (3 mL) were added (3-fluoro-4-nitrophenyl) methanol (100 mg, 0.58 mmol, 1.0 eq.) and TEA (473.04 mg, 4.68 mmol, 8.0 eq.) at −40° C. After addition, the mixture was stirred at this temperature for 3 hrs. 2-bromopropan-1-aminehydrobromide (570.23 mg, 4.68 mmol, 8.0 eq.) and TEA (473.04 mg, 4.68 mmol, 8.0 eq.) were added into the mixture. After addition, the reaction mixture was stirred at −40° C. for another 1 hr. After completion, the reaction mixture was warmed up at room temperature, quenched with sat. NH₄Cl solution (10 mL) and extracted with DCM (10 mL). The organic phase was washed with water (10 mL), dried over anhydrous Na₂SO₄, filtered and concentrated under reduced pressure. The residue was purified by Biotage® C18 column chromatography to give 3-fluoro-4-nitrobenzyl di(2-bromopropan-1-yl amino)phosphinate (180 mg, 0.37 mmol, 63%) as a yellow solid. ¹H NMR (400 MHz, CDCl₃) δ 8.17-7.90 (m, 1H), 7.32 (dd, J=18.3, 10.2 Hz, 2H), 5.10 (d, J=8.0 Hz, 2H), 4.29-4.06 (m, 2H), 3.33-3.31 (m, 2H), 3.15-3.10 (m, 4H), 1.70 (dd, J=6.7, 1.3 Hz, 6H).

3-fluoro-4-nitrobenzyl bis(2-methylaziridin-1-yl)phosphinate

To a solution of 3-fluoro-4-nitrobenzyl di(2-bromopropan-1-yl amino)phosphinate (180 mg, 0.37 mmol, 1.0 eq.) and DIEA (236.85 mg, 1.83 mmol, 5.0 eq.) in THF (20 mL) was added Ag₂O (424.7 mg, 1.83 mmol, 5.0 eq.). The mixture was stirred at 65° C. for 16 hrs. After completion, the reaction mixture was filtered. The filtrate was concentrated under reduced pressure. The residue was purified by Biotage® C18 column chromatography to give (3-fluoro-4-nitrophenyl)methyl bis(2-methylaziridin-1-yl)phosphinate (70 mg, 0.21 mmol, 58%) as a colorless oil. LC-MS (ESI): m/z 330.1 [M+H]⁺.

3-(3-(dimethylcarbamoyl)phenoxy)-4-nitrobenzyl bis(2-methylaziridin-1-yl)phosphinate

To a solution of (3-fluoro-4-nitrophenyl)methyl bis(2-methylaziridin-1-yl)phosphinate (35 mg, 0.11 mmol, 1.0 eq.) and Cs₂CO₃ (86.58 mg, 0.27 mmol, 2.5 eq.) in MeCN (3 mL) was added 3-hydroxy-N,N-dimethylbenzamide (35.12 mg, 0.21 mmol, 1.9 eq.) and the mixture was stirred at 80° C. for 2 hrs. After completion, the reaction mixture was cooled to room temperature and filtered. The filtrate was concentrated under reduced pressure to give a residue. The residue was purified by RP-prep HPLC to give 3-(3-(dimethylcarbamoyl)phenoxy)-4-nitrobenzyl bis(2-methylaziridin-1-yl)phosphinate (5.4 mg, 10.5 μmol, 11%) as a yellow oil. ¹H NMR (400 MHz, CDCl₃) δ 7.99 (dd, J=8.4, 1.8 Hz, 1H), 7.45-7.39 (m, 1H), 7.28 (d, J=1.9 Hz, 1H), 7.23 (d, J=7.7 Hz, 1H), 7.10-7.05 (m, 3H), 5.13 (dd, J=7.6, 3.8 Hz, 2H), 3.09 (s, 3H), 2.98 (s, 3H), 2.54-2.52 (m, 2H), 2.38-2.24 (m, 2H), 1.90-1.88 (m, 1H), 1.82-1.80 (m, 1H), 1.28-1.24 (m, 3H), 1.23-1.20 (m, 3H). ³¹P NMR (162 MHz, CDCl₃) δ 29.83 (t, J=26.0 Hz). LC-MS (ESI): m/z 475.2 [M+H]⁺.

### Illustration 31. Synthesis of (S)-1-(3-(3-(dimethylcarbamoyl)phenoxy)-4-nitrophenyl)ethyl bis((S)-2-methylaziridin-1-yl)phosphinate (209) (General Procedure 2)

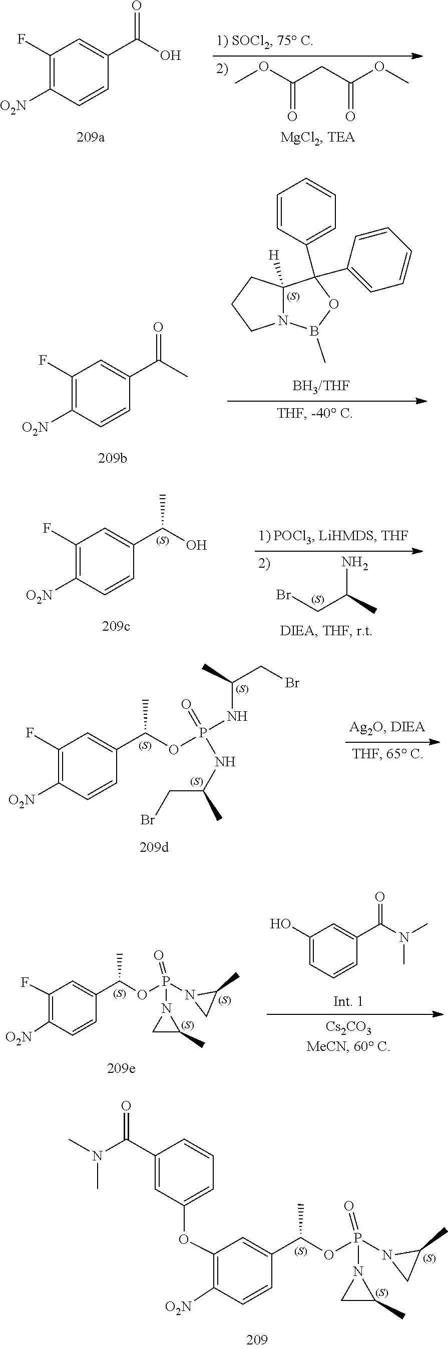

1-(3-fluoro-4-nitrophenyl) ethan-1-one

To a solution of 3-fluoro-4-nitrobenzoic acid (12 g, 64.83 mmol, 1.0 eq.) in SOCl₂ (40 mL) was added DMF (0.5 mL) and the mixture was stirred at 75° C. for 3 hrs. After completion, the mixture was concentrated under reduced pressure to give a yellow solid. The yellow solid was dissolved in toluene (120 mL) to give a solution. A mixture of MgCl₂ (3.7 g, 38.90 mmol, 0.6 eq.), TEA (18.8 mL, 135.25 mmol, 2.0 eq.) and 1,3-dimethyl propanedioate (7.6 mL, 66.45 mmol, 1.0 eq.) was stirred at room temperature for 1.5 hrs followed by addition of the above solution. The resulting mixture was stirred at room temperature for another 1.5 hrs, then conc. HCl (16 mL) was added and the reaction mixture was stirred for 10 min. The mixture was extracted with EtOAc (100 mL×2). The combined organic layers were washed with brine (50 mL), dried over anhydrous Na₂SO₄, filtered and concentrated under reduced pressure. The residue was purified by flash column chromatography on silica gel to give 1-(3-fluoro-4-nitrophenyl) ethan-1-one (7.4 g, 40.41 mmol, 62%) as a yellow solid. ¹H NMR (400 MHz, CDCl₃) δ 8.21-8.08 (m, 1H), 7.93-7.75 (m, 2H), 2.68 (s, 3H).

(S)-1-(3-fluoro-4-nitrophenyl) ethan-1-ol

To a solution of (3aS)-1,3a-dimethyl-3,3-diphenyl-hexahydropyrrolo[1,2-c][1,3,2]oxazaborole (0.87 g, 3.00 mmol, 0.3 eq.) in toluene (3 mL) was added BH₃·THF (11 mL, 1 N, 11.00 mmol, 1.1 eq.) at 0° C. under nitrogen. The solution was stirred at 0° C. for 30 min, then cooled to −40° C. A solution of 1-(3-fluoro-4-nitrophenyl) ethan-1-one (1.83 g, 9.99 mmol, 1.0 eq.) in THF (40 mL) was added slowly to the above mixture during 1 hr at −40° C. After addition, the reaction mixture was stirred at −40° C. for 2 hrs. MeOH (20 mL) was added into the reaction mixture at −40° C., and the solution was stirred for 30 min. The mixture was concentrated under reduced pressure. The residue was purified by flash column chromatography on silica gel and SFC to give (S)-1-(3-fluoro-4-nitrophenyl) ethan-1-ol (1.7 g, 9.18 mmol, 92%) as a yellow solid. ¹H NMR (400 MHz, CDCl₃) δ 8.12-7.96 (m, 1H), 7.37-7.32 (m, 1H), 7.30-7.27 (m, 1H), 4.99 (q, J=6.4 Hz, 1H), 1.52 (d, J=6.5 Hz, 3H).

(S)-1-(3-fluoro-4-nitrophenyl)ethyl di((S)-1-bromopropan-2-yl amino)phosphinate

To a solution of (S)-1-(3-fluoro-4-nitrophenyl) ethan-1-ol (200 mg, 1.08 mmol, 1.0 eq.) in THF (10 mL) was added LiHMDS (1.6 mL, 1N, 1.60 mmol, 1.5 eq.) at −78° C. After addition, the mixture was stirred for 20 min at this temperature. Then, POCl₃ (0.201 mL, 2.16 mmol, 2.0 eq.) was added and the mixture was stirred at −78° C. for 20 min, (S)-1-bromopropan-2-amine hydrobromide (1.2 g, 5.4 mmol, 5.0 eq.) and DIEA (1.43 mL, 8.64 mmol, 8.0 eq.) were added into the mixture. After addition, the reaction mixture was warmed to room temperature and stirred for 1 hr. After completion, the reaction mixture was quenched with sat. NH₄Cl solution (10 mL) and extracted with DCM (20 mL). The organic phase was washed with water (10 mL), dried over anhydrous Na₂SO₄, filtered and concentrated under reduced pressure. The residue was purified by Biotage® C18 column chromatography to give (S)-1-(3-fluoro-4-nitrophenyl)ethyl di((S)-1-bromopropan-2-yl amino)phosphinate (100 mg, 0.20 mmol, 18%) as a yellow oil. LC-MS (ESI): m/z 504 [M+H]⁺.

(S)-1-(3-fluoro-4-nitrophenyl)ethyl bis((S)-2-methylaziridin-1-yl)phosphinate

A mixture of (S)-1-(3-fluoro-4-nitrophenyl)ethyl di((S)-1-bromopropan-2-yl amino)phosphinate (100 mg, 0.20 mmol, 1.0 eq.), Ag₂O (229 mg, 0.99 mmol, 5.0 eq.) and DIEA (204 mg, 1.58 mmol, 7.9 eq.) in THF (10 mL) was stirred at 65° C. for 16 hrs under nitrogen. After completion, the reaction mixture was cooled to room temperature and filtered. The filtrate was concentrated under reduced pressure to give a residue. The residue was purified by Biotage® C18 column chromatography to give (S)-1-(3-fluoro-4-nitrophenyl)ethyl bis((S)-2-methylaziridin-1-yl)phosphinate (30 mg, 0.09 mmol, 44%) as a colorless oil. LC-MS (ESI): m/z 344.1 [M+H]⁺. ¹H NMR (400 MHz, CDCl₃) δ 8.13-8.01 (m, 1H), 7.37-7.29 (m, 2H), 5.74-5.60 (m, 1H), 2.58-2.45 (m, 2H), 2.39-2.19 (m, 2H), 1.94-1.90 (m, 1H), 1.77-1.73 (m, 1H), 1.62 (d, J=6.6 Hz, 3H), 1.24-1.21 (m, 3H), 1.16-1.15 (m, 3H).

(S)-1-(3-(3-(dimethylcarbamoyl)phenoxy)-4-nitrophenyl)ethyl bis((S)-2-methylaziridin-1-yl)phosphinate

A mixture of (S)-1-(3-fluoro-4-nitrophenyl)ethyl bis((S)-2-methylaziridin-1-yl)phosphinate (30 mg, 0.087 mmol, 1.0 eq.), 3-hydroxy-N,N-dimethylbenzamide (28.9 mg, 0.175 mmol, 2.0 eq.) and Cs₂CO₃ (71.2 mg, 0.22 mmol, 2.5 eq.) in MeCN (5 mL) was stirred at 60° C. for 1 hr. After completion, the mixture was cooled to room temperature and filtered. The filtrate was concentrated under reduced pressure to give a residue. The residue was purified by Biotage® C18 column chromatography to give (S)-1-(3-(3-(dimethylcarbamoyl)phenoxy)-4-nitrophenyl)ethyl bis((S)-2-methylaziridin-1-yl)phosphinate (38.9 mg, 0.08 mmol, 91%) as a yellow oil. ¹H NMR (400 MHz, CDCl₃) δ 7.99 (d, J=8.5 Hz, 1H), 7.44-7.38 (m, 1H), 7.31-7.28 (m, 1H), 7.24-7.22 (m, 1H), 7.11-7.05 (m, 3H), 5.63-5.55 (m, 1H), 3.10 (s, 3H), 2.99 (s, 3H), 2.54-2.40 (m, 2H), 2.35-2.18 (m, 2H), 1.91-1.87 (m, 1H), 1.73-1.56 (m, 1H), 1.57 (d, J=6.6 Hz, 3H), 1.28-1.20 (m, 3H), 1.18-1.13 (m, 3H). ³¹P NMR (162 MHz, CDCl₃) δ 29.06 (s). LC-MS (ESI): m/z 489.2 [M+H]⁺.

### Illustration 32. Synthesis of (R)-1-(3-(3-(dimethylcarbamoyl)phenoxy)-4-nitrophenyl)-2,2,2-trifluoroethyl bis((S)-2-methylaziridin-1-yl)phosphinate (225) (General Procedure 3)

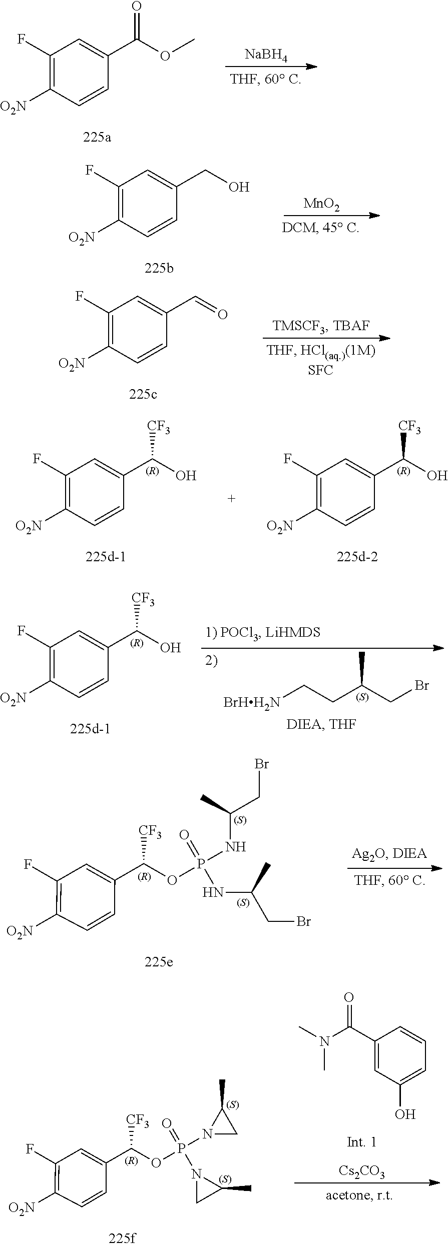

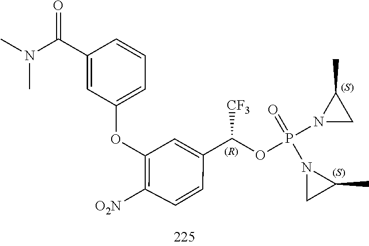

(3-fluoro-4-nitrophenyl)methanol

To a solution of methyl 3-fluoro-4-nitrobenzoate (800 mg, 4.02 mmol, 1.0 eq.) in THF (20 mL) was added NaBH₄ (1.2 g, 32.2 mmol, 8.0 eq.) in portions under nitrogen and then the reaction mixture was stirred at 60° C. for 18 hrs. After completion, the reaction mixture was quenched by adding sat. NH₄Cl aqueous solution (20 mL), then extracted with EtOAc (20 mL×3). The organic layers were combined and washed with brine (20 mL), dried over anhydrous Na₂SO₄, and concentrated under reduced pressure. The residue was purified by Biotage® C18 column chromatography to afford (3-fluoro-4-nitrophenyl) methanol (690 mg, 4.01 mmol, 98%) as a white solid. LC-MS (ESI) m/z 172 [M+H]⁺. ¹HNMR (400 MHz, CDCl₃) δ 8.09-8.02 (m, 1H), 7.34 (d, J=11.7 Hz, 1H), 7.28-7.24 (m, 1H), 4.81 (s, 2H), 2.07 (s, 1H).

fluoro-4-nitrobenzaldehyde

To a solution of (3-fluoro-4-nitrophenyl) methanol (2.5 g, 14.61 mmol, 1.0 eq.) in DCM (50 mL) was added MnO₂ (12.7 g, 146.09 mmol, 10.0 eq.) at room temperature. After addition, the reaction mixture was stirred at 45° C. overnight. After completion, the reaction mixture was cooled to room temperature and filtered. The filtrate was concentrated under reduced pressure to give 3-fluoro-4-nitrobenzaldehyde (2.4 g, 14.19 mmol, 97%) as a white solid, which was used in next step directly without further purification. ¹H NMR (400 MHz, CDCl₃) δ 10.09 (d, J=1.5 Hz, 1H), 8.24-8.20 (m, 1H), 7.89-7.73 (m, 2H).

(R)-2,2,2-trifluoro-1-(3-fluoro-4-nitrophenyl) ethan-1-ol(S)-2,2,2-trifluoro-1-(3-fluoro-4-nitrophenyl) ethan-1-ol

To a solution of 1-(3-fluoro-4-nitrophenyl) ethan-1-one (590 mg, 3.22 mmol, 1.0 eq.) and TMSCF₃ (744.17 mg, 5.23 mmol, 1.6 eq.) in THF (10 mL) was added TBAF (0.140 mL, 0.14 mmol, 0.04 eq.) at 0° C. After addition, the reaction mixture was stirred at 0° C. for 30 min. Then, 3N HCl solution (0.6 mL, aq.) was added and the reaction mixture was stirred at 0° C. for 1 hr. The mixture was extracted with DCM (30 mL). The organic phase was washed with water (10 mL×3), dried over anhydrous Na₂SO₄, filtered and concentrated under reduced pressure. The residue was purified by flash column chromatography on silica gel to give 2,2,2-trifluoro-1-(3-fluoro-4-nitrophenyl) ethan-1-ol (400 mg, 1.67 mmol, 48%) as a yellow oil.

2,2,2-trifluoro-1-(3-fluoro-4-nitrophenyl) ethan-1-ol (1.69 g, 7.10 mmol) was purified by SFC to afford (R)-2,2,2-trifluoro-1-(3-fluoro-4-nitrophenyl) ethan-1-ol (0.9 g, 3.76 mmol, 53% yield, 99.6% ee) and(S)-2,2,2-trifluoro-1-(3-fluoro-4-nitrophenyl) ethan-1-ol (0.8 g, 3.34 mmol, 47% yield, 100% ee). ¹H NMR (400 MHz, CDCl₃) δ 8.13-8.09 (m, 1H), 7.52-7342 (m, 2H), 5.23-5.06 (m, 1H), 2.98 (d, J=4.5 Hz, 1H). ¹⁹F NMR (376 MHz, CDCl₃) δ −78.13 (s), −116.16 (s).

(R)-2,2,2-trifluoro-1-(3-fluoro-4-nitrophenyl)ethyl di((S)-1-bromopropan-2-yl amino)phosphinate

To a solution of (R)-2,2,2-trifluoro-1-(3-fluoro-4-nitrophenyl) ethan-1-ol (120 mg, 0.50 mmol, 1.0 eq.) in THF (10 mL) was added LiHMDS (0.64 mL, 0.64 mmol, 1.5 eq.) at −78° C. under nitrogen. After addition, the mixture was stirred at this temperature for 30 min. Then, POCl₃ (0.094 mL, 1.00 mmol, 2.0 eq.) was added and the mixture was stirred at −78° C. for 30 min, (S)-1-bromopropan-2-amine hydrobromide (416 mg, 3.01 mmol, 6.0 eq.) and TEA (0.56 mL, 4.02 mmol 8.0 eq.) were added into the mixture. After addition, the reaction mixture was warmed to room temperature and stirred for 2 hrs. After completion, the reaction mixture was quenched with sat. NH₄Cl solution (10 mL), extracted with DCM (10 mL×2). The combined organic phases were washed with water (10 mL), dried over anhydrous Na₂SO₄, filtered and concentrated under reduced pressure. The residue was purified by Biotage® C18 column chromatography to give (R)-2,2,2-trifluoro-1-(3-fluoro-4-nitrophenyl)ethyl di((S)-1-bromopropan-2-yl amino)phosphinate (70 mg, 0.125 mmol, 25%) as a yellow oil. LC-MS (ESI): m/z 558 [M+H]⁺.

(R)-2,2,2-trifluoro-1-(3-fluoro-4-nitrophenyl)ethyl bis((S)-2-methylaziridin-1-yl)phosphinate

A solution of (R)-2,2,2-trifluoro-1-(3-fluoro-4-nitrophenyl)ethyl di((S)-1-bromopropan-2-yl amino)phosphinate (70 mg, 0.13 mmol, 1.0 eq.), DIEA (0.103 mL, 0.63 mmol, 5.0 eq.) and Ag₂O (0.021 mL, 0.63 mmol, 5.0 eq.) in THF (4 mL) was stirred at 60° C. for 12 hrs. After completion, the suspension was filtered through a pad of Celite® and the filtrate was concentrated under reduced pressure. The residue was purified by Biotage® C18 column chromatography to give (R)-2,2,2-trifluoro-1-(3-fluoro-4-nitrophenyl)ethyl bis((S)-2-methylaziridin-1-yl)phosphinate (48 mg, 0.12 mmol, 97%) as a light yellow oil. LC-MS (ESI): m/z 398 [M+H]⁺.

(R)-1-(3-(3-(dimethylcarbamoyl)phenoxy)-4-nitrophenyl)-2,2,2-trifluoroethyl bis((S)-2-methylaziridin-1-yl)phosphinate

To a solution of (R)-2,2,2-trifluoro-1-(3-fluoro-4-nitrophenyl)ethyl bis((S)-2-methylaziridin-1-yl)phosphinate (48 mg, 0.12 mmol, 1.0 eq.) and Cs₂CO₃ (98 mg, 0.30 mmol, 2.5 eq.) in acetone (4 mL) was added 3-hydroxy-N,N-dimethylbenzamide (40 mg, 0.24 mmol, 2.0 eq.) at room temperature. The reaction mixture was stirred at room temperature for 5 hrs. After completion, the reaction mixture was filtered and the filtrate was concentrated under reduced pressure. The residue was purified by Biotage® C18 column chromatography to give (R)-1-(3-(3-(dimethylcarbamoyl)phenoxy)-4-nitrophenyl)-2,2,2-trifluoroethyl bis((S)-2-methylaziridin-1-yl)phosphinate (15 mg, 28 μmol, 23%) as a yellow solid. ¹H NMR (400 MHz, CDCl₃) δ 8.03 (d, J=7.8 Hz, 1H), 7.50-7.36 (m, 2H), 7.29-7.18 (m, 2H), 7.17-7.01 (m, 2H), 5.85-5.58 (m, 1H), 3.11 (s, 3H), 3.00 (s, 3H), 2.69-2.55 (m, 1H), 2.51-2.33 (m, 2H), 2.25-2.13 (m, 1H), 2.02-1.73 (m, 2H), 1.35-1.05 (m, 6H). ¹⁹F NMR (376 MHz, CDCl₃) δ −76.81 (d, J=19.4 Hz). ³¹P NMR (162 MHz, CDCl₃) δ 30.99 (d, J=8.8 Hz). LC-MS (ESI): m/z 543.2 [M+H]⁺.

### Illustration 33. Synthesis of (5-(3-(dimethylcarbamoyl)phenoxy)-6-nitrobenzofuran-2-yl)methyl di(aziridin-1-yl)phosphinate (229) (Formular II-1)

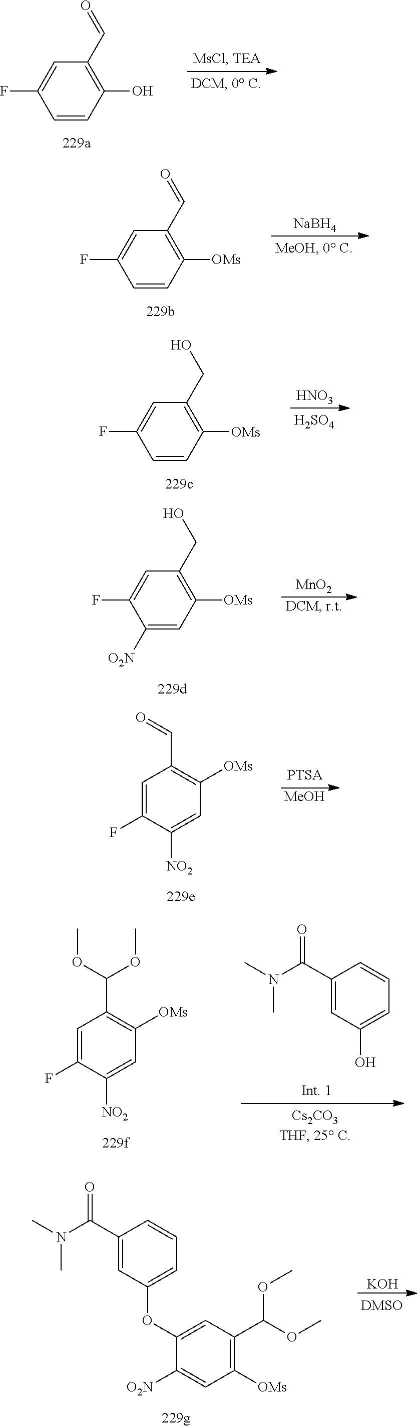

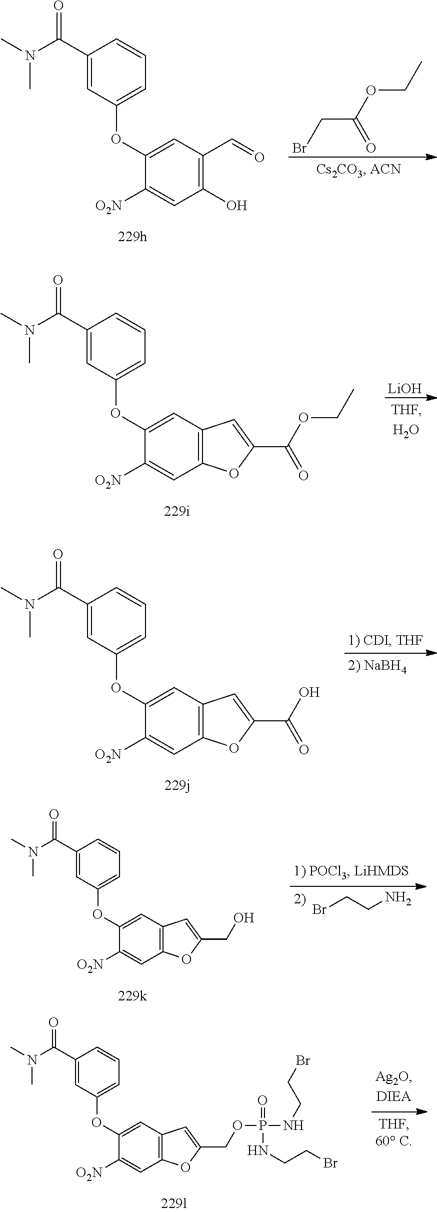

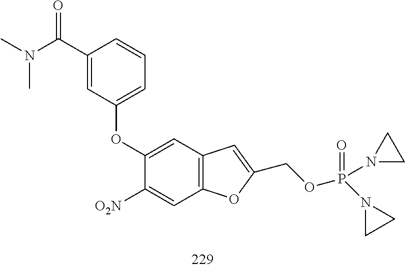 [SMILES: CN(C)C(=O)c1cccc(Oc2cc3cc(COP(=O)(N4CC4)N4CC4)oc3cc2[N+](=O)[O-])c1]

4-fluoro-2-formylphenyl methanesulfonate

To a solution of 5-fluoro-2-hydroxybenzaldehyde (5.0 g, 35.7 mmol) in DCM (50 mL) were added TEA (8.93 mL, 64.2 mmol) and MsCl (3.04 mL, 39.3 mmol) at 0° C., and the reaction mixture was stirred at 0° C. for 20 min. After completion, the reaction mixture was quenched by adding NH₄Cl (20 mL, aq.), then extracted with DCM (30 mL×3). The organic layers were combined, washed with brine, dried over anhydrous Na₂SO₄ and concentrated under reduced pressure to give 4-fluoro-2-formylphenyl methanesulfonate (7.5 g, 34.4 mmol, 96%) as a yellow oil, which was used in next step directly without further purification. 1H NMR (400 MHz, CDCl₃) δ 10.24 (d, J=2.6 Hz, 1H), 7.64 (dd, J=7.9, 3.1 Hz, 1H), 7.44 (dd, J=9.0, 4.3 Hz, 1H), 7.40-7.33 (m, 1H), 3.32 (s, 3H).

4-fluoro-2-(hydroxymethyl)phenyl methanesulfonate

To a solution of 4-fluoro-2-formylphenyl methanesulfonate (7.50 g, 34.371 mmol) in MeOH (100 mL) was added NaBH₄ (2.01 g, 52.9 mmol) and the reaction mixture was stirred at 0° C. for 30 min. After completion, the reaction mixture was quenched by adding H₂O (100 mL), then extracted with EtOAc (80 mL×3). The organic layers were combined, dried over anhydrous Na₂SO₄ and concentrated under reduced pressure to give 4-fluoro-2-(hydroxymethyl)phenyl methanesulfonate (7.0 g, 32.1 mmol, 93%) as a yellow oil, which was used in next step directly without further purification. ¹H NMR (400 MHz, CDCl₃) δ 7.30-7.26 (m, 2H), 7.08-7.00 (m, 1H), 4.72 (s, 2H), 3.20 (s, 3H).

4-fluoro-2-(hydroxymethyl)-5-nitrophenyl methanesulfonate

To a solution of 4-fluoro-2-(hydroxymethyl)phenyl methanesulfonate (6.90 g, 31.3 mmol) in H₂SO₄ (2.5 mL) were added HNO₃ (2.2 mL) and H₂SO₄ (2.5 mL) at 0° C., and the reaction mixture was stirred at room temperature for 4 hrs. After completion, the reaction mixture was quenched by adding ice water (20 mL), then extracted with EtOAc (20 mL×3). The organic layers were combined and washed with brine, dried over anhydrous Na₂SO₄, and concentrated under reduced pressure to give 4-fluoro-2-(hydroxymethyl)-5-nitrophenyl methanesulfonate (8.7 g, crude), which was used in next step directly without further purification. ¹H NMR (400 MHz, CDCl₃) δ 8.03 (d, J=6.2 Hz, 1H), 7.61 (d, J=11.0 Hz, 1H), 4.86 (d, J=5.3 Hz, 2H), 3.34 (s, 3H), 2.31-2.15 (m, 1H).

2-(dimethoxymethyl)-4-fluoro-5-nitrophenyl methanesulfonate

To a solution of 4-fluoro-2-(hydroxymethyl)-5-nitrophenyl methanesulfonate (3.5 g, 13.2 mmol) in DCM (50 mL) was added MnO₂ (15.5 g, 178 mmol) at 25° C. and the resulting mixture was stirred at 25° C. for 16 hrs. The mixture was filtered through a pad of Celite®, and the filter cake was washed with DCM (50 mL), The combined filtrates were concentrated under reduced pressure. The residue was heated under reflux for 1 hr with 100 mL of methanol and 0.25 g of p-toluene sulfonic acid monohydrate. NaHCO₃ (20 mL) was added to the solution, and the solution was extracted with DCM (40 mL×3). The organic layers were combined, dried over anhydrous Na₂SO₄ and concentrated under reduced pressure to give 2-(dimethoxymethyl)-4-fluoro-5-nitrophenyl methanesulfonate (3.5 g, 11.32 mmol, 86%) as a yellow solid. ¹H NMR (400 MHz, CDCl₃) δ 8.10 (d, J=6.2 Hz, 1H), 7.64 (d, J=11.0 Hz, 1H), 5.64 (s, 1H), 3.36 (s, 6H), 3.32 (s, 3H).

2-(dimethoxymethyl)-4-(3-(dimethylcarbamoyl)phenoxy)-5-nitrophenyl methanesulfonate

To a solution of 2-(dimethoxymethyl)-4-fluoro-5-nitrophenyl methanesulfonate (1.50 g, 4.85 mmol) and 3-hydroxy-N,N-dimethylbenzamide (0.96 g, 5.82 mmol) in THF (10 mL) was added CS₂CO₃ (3.16 g, 9.70 mmol) at 0° C. and the resulting mixture was stirred at 25° C. for 16 hrs. After completion, the reaction mixture was quenched with H₂O (40 mL) and extracted with EtOAc (30 mL×3). The organic layers were combined and washed with brine, dried over anhydrous Na₂SO₄, and concentrated under reduced pressure. The residue was purified by flash column chromatography on silica gel to afford 2-(dimethoxymethyl)-4-(3-(dimethylcarbamoyl)phenoxy)-5-nitrophenyl methanesulfonate (1.65 g, 3.63 mmol, 75%) as a yellow oil.

3-(5-formyl-4-hydroxy-2-nitrophenoxy)-N,N-dimethylbenzamide

To a solution of 2-(dimethoxymethyl)-4-[3-(dimethylcarbamoyl)phenoxy]-5-nitrophenyl methanesulfonate (1.65 g, 3.63 mmol) in DMSO (18 mL) was added KOH (0.33 g, 5.81 mmol) and the resulting mixture was stirred at room temperature for 2 hrs. The reaction mixture was then neutralized carefully with HCl (aq.) until the pH was adjusted to pH=5-6. The solution was extracted with EtOAc (60 mL×3). The organic layers were combined and washed with brine, dried over anhydrous Na₂SO₄, and concentrated under reduced pressure to give 3-(5-formyl-4-hydroxy-2-nitrophenoxy)-N,N-dimethylbenzamide (700 mg, 2.12 mmol, 58%) as a yellow oil. ¹H NMR (400 MHz, CDCl₃) δ 10.88 (s, 1H), 9.87 (s, 1H), 7.48 (s, 1H), 7.43-7.36 (m, 2H), 7.20 (d, J=7.6 Hz, 1H), 7.09-7.05 (m, 1H), 7.02 (d, J=1.3 Hz, 1H), 3.10 (s, 3H), 2.97 (s, 3H).

ethyl 5-(3-(dimethylcarbamoyl)phenoxy)-6-nitrobenzofuran-2-carboxylate

To a solution of 3-(5-formyl-4-hydroxy-2-nitrophenoxy)-N,N-dimethylbenzamide (700 mg, 0.82 mmol) and ethyl 2-bromoacetate (0.282 mL, 0.981 mmol) in ACN (5 mL) was added CS₂CO₃ (2.07 g, 2.45 mmol) at 0° C., then the resulting mixture was warmed up to 25° C. and stirred for 16 hrs. After completion, the reaction mixture was diluted with H₂O (20 mL), extracted with EtOAc (20 mL×3). The organic layers were combined and washed with brine, dried over anhydrous Na₂SO₄, and concentrated under reduced pressure to give ethyl 5-[3-(dimethylcarbamoyl)phenoxy]-6-nitro-1-benzofuran-2-carboxylate (388 mg, 0.38 mmol, 46%) as a yellow oil. ¹H NMR (400 MHz, CDCl₃) δ 8.21 (d, J=0.6 Hz, 1H), 7.47 (dd, J=5.3, 1.4 Hz, 1H), 7.40 (d, J=3.6 Hz, 1H), 7.37-7.31 (m, 1H), 7.22-7.13 (m, 1H), 7.12-7.06 (m, 1H), 7.03 (m, 1H), 7.00-6.94 (m, 1H), 4.53-4.42 (m, 2H), 3.08 (s, 3H), 2.96 (s, 3H), 1.31-1.25 (m, 3H).

5-(3-(dimethylcarbamoyl)phenoxy)-6-nitrobenzofuran-2-carboxylic acid

To a solution of ethyl 5-[3-(dimethylcarbamoyl)phenoxy]-6-nitro-1-benzofuran-2-carboxylate (390 mg, 0.138 mmol) in THF (9 mL) and H₂O (3 mL) was added LiOH (0.086 mL, 0.41 mmol) at 25° C. and the resulting mixture was stirred at 25° C. for 16 hrs. After completion, the reaction mixture was cooled down in an ice bath, then neutralized carefully with HCl (aq.) until the pH was adjusted to pH=4-5. The resulting mixture was extracted with EtOAc (20 mL×3). The combined organic layers were washed with brine (10 mL×2), dried over with anhydrous Na₂SO₄, and concentrated under reduced pressure to give crude 5-(3-(dimethylcarbamoyl)phenoxy)-6-nitrobenzofuran-2-carboxylic acid (142 mg, 0.054 mmol, 39%) as a yellow oil, which was used in next step directly without further purification. LC-MS (EST): m/z 371 [M+H]⁺.

3-((2-(hydroxymethyl)-6-nitrobenzofuran-5-yl)oxy)-N,N-dimethylbenzamide

To a solution of 5-(3-(dimethylcarbamoyl)phenoxy)-6-nitrobenzofuran-2-carboxylic acid (150 mg, 0.14 mmol) in THF (9 mL) was added CDI (26.2 mg, 0.16 mmol) and the resulting mixture was stirred at 25° C. for 1 hr. NaBH₄ (0.027 mL, 0.270 mmol) and H₂O (3 mL) was added to the solution, then the mixture was stirred at 25° C. for additional 2 hrs. After completion, the reaction mixture was quenched by adding NH₄Cl (40 mL, aq.), then extracted with EtOAc (40 mL×3). The organic layers were combined and washed with brine (150 mL), dried over anhydrous Na₂SO₄, and concentrated under reduced pressure. The residue was purified by C18 column to give 3-((2-(hydroxymethyl)-6-nitrobenzofuran-5-yl)oxy)-N,N-dimethylbenzamide (60 mg, 0.056 mmol, 42%) as a yellow oil. LC-MS (ESI): m/z 357 [M+H]⁺.

(5-(3-(dimethylcarbamoyl)phenoxy)-6-nitrobenzofuran-2-yl)methyl di((2-bromoethyl)amino)phosphinate

To a solution of 3-{[2-(hydroxymethyl)-6-nitro-1-benzofuran-5-yl]oxy}-N,N-dimethylbenzamide (110 mg, 0.309 mmol) in THF (20 mL) was added LiHMDS (0.37 mL, 0.37 mmol) at 0° C. The mixture was stirred for additional 0.5 hrs. POCl₃ (0.058 mL, 0.62 mmol) was added into the mixture and the reaction mixture was stirred for 1 h, then 2-bromoethan-1-amine (0.143 mL, 1.85 mmol) and TEA (0.26 mL, 1.85 mmol) were added into the mixture, then the mixture was stirred at room temperature for 1 hr. After completion, the reaction mixture was quenched with sat. NH₄Cl solution and extracted with EtOAc. The organic layer was washed with brine, dried over anhydrous Na₂SO₄ and concentrated under reduced pressure. The residue was purified by C18 column to afford (5-(3-(dimethylcarbamoyl)phenoxy)-6-nitrobenzofuran-2-yl)methyl di((2-bromoethyl)amino)phosphinate (60 mg, 0.093 mmol, 30%) as a yellow oil. LC-MS (ESI): m/z 647 [M+H]⁺.

(5-(3-(dimethylcarbamoyl)phenoxy)-6-nitrobenzofuran-2-yl)methyl di(aziridin-1-yl)phosphinate

To a solution of (5-(3-(dimethylcarbamoyl)phenoxy)-6-nitrobenzofuran-2-yl)methyl di((2-bromoethyl)amino)phosphinate (60 mg, 0.093 mmol) in THF (10 mL) were added Ag₂O (0.015 mL, 0.463 mmol) and DIEA (0.076 mL, 0.463 mmol). The resulting mixture was stirred at 60° C. overnight. After completion, the mixture was filtered and the filtrates were purified by C18 column to give (5-(3-(dimethylcarbamoyl)phenoxy)-6-nitrobenzofuran-2-yl)methyl di(aziridin-1-yl)phosphinate (10 mg, 0.021 mmol, 22%) as a yellow oil. ¹H NMR (400 MHz, CDCl₃) δ 8.17 (s, 1H), 7.39 (t, J=7.9 Hz, 1H), 7.29 (s, 1H), 7.18-7.14 (m, 1H), 7.07 (dd, J=8.2, 1.8 Hz, 1H), 7.00-6.97 (m, 1H), 6.81 (s, 1H), 5.26 (d, J=8.4 Hz, 2H), 3.08 (s, 3H), 2.96 (s, 3H), 2.28-2.15 (m, 8H). ³¹P NMR (162 MHz, CDCl₃) δ 31.03 (s). LC-MS (ESI): m/z 487.2 [M+H]⁺.

### Illustration 34. Synthesis of (5-([1,1′-biphenyl]-4-yloxy)-6-nitrobenzofuran-2-yl)methyl di(aziridin-1-yl)phosphinate (230)

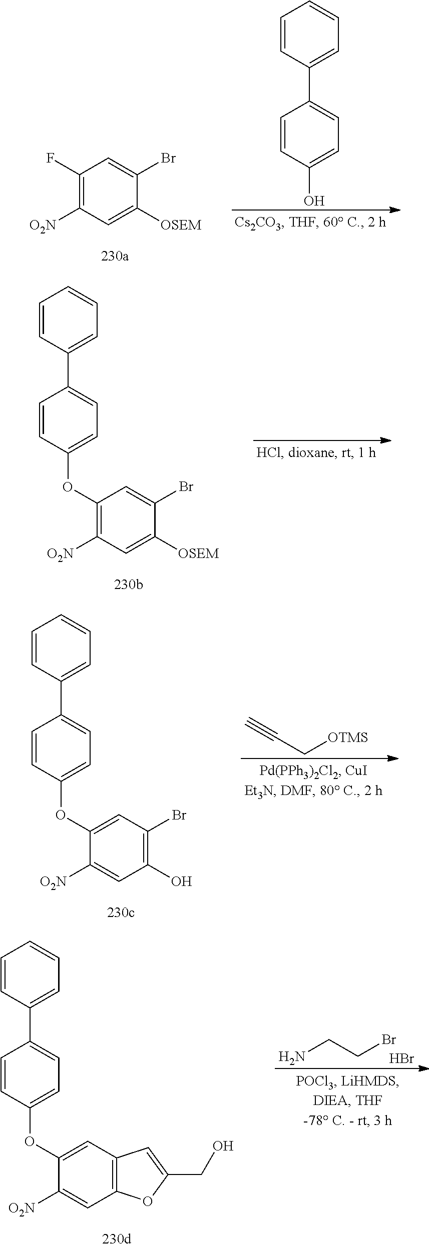

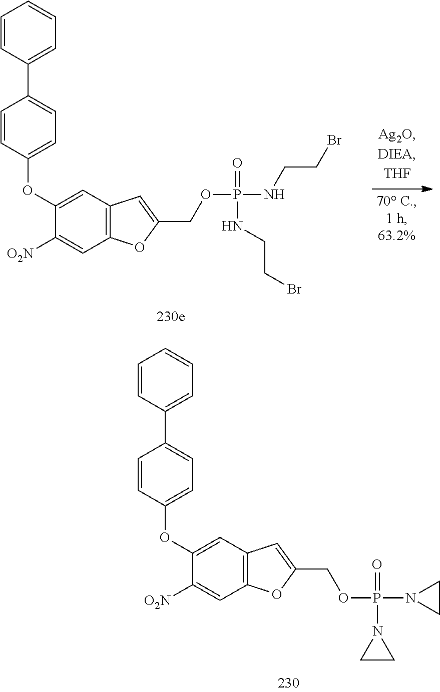 [SMILES: O=[N+]([O-])c1cc2oc(COP(=O)(NCCBr)NCCBr)cc2cc1Oc1ccc(-c2ccccc2)cc1 ; O=[N+]([O-])c1cc2oc(COP(=O)(N3CC3)N3CC3)cc2cc1Oc1ccc(-c2ccccc2)cc1]

((4-([1,1′-biphenyl]-4-yloxy)-2-bromo-5-nitrophenoxy)methoxy)ethyl)trimethylsilane

To a stirred solution [2-(2-bromo-4-fluoro-5-nitrophenoxymethoxy)ethyl]trimethylsilane (1.00 g, 2.73 mmol, 1.00 equiv) and phenylphenol (560 mg, 3.28 mmol, 1.20 equiv) in THF (100 mL) was added Cs₂CO₃ (1.78 g, 5.46 mmol, 2.00 equiv). The resulting mixture was stirred for 2 h at 60° C. The reaction was quenched by the addition of water (100 mL) and extracted with EtOAc (3× 100 mL). The combined organic layers were washed with brine (2×50 mL), dried over anhydrous na₂SO₄. After filtration, the filtrate was concentrated under reduced pressure. The residue was purified by silica gel column chromatography, eluted with PE/EA (10:1) to afford (2-((4-([1,1′-biphenyl]-4-yloxy)-2-bromo-5-nitrophenoxy)methoxy)ethyl)trimethylsilane (1.00 g, 70.9%) as a white solid.

4-([1,1′-biphenyl]-4-yloxy)-2-bromo-5-nitrophenol

To a stirred solution (2-((4-([1,1′-biphenyl]-4-yloxy)-2-bromo-5-nitrophenoxy)methoxy)ethyl)trimethylsilane (1.80 g, 3.48 mmol, 1.00 equiv) in HCl (gas) (100 mL, 4.0 M in 1,4-dioxane). The resulting mixture was stirred for 1 h at room temperature. Then the mixture was concentrated under reduced pressure. The residue was purified by silica gel column chromatography, eluted with PE/EA (3:1) to afford 4-([1,1′-biphenyl]-4-yloxy)-2-bromo-5-nitrophenol (1.03 g, 76.5%) as a yellow solid. LC-MS (ESI): m/z 383.90, 385.90 [M−H]⁻

(5-([1,1′-biphenyl]-4-yloxy)-6-nitrobenzofuran-2-yl) methanol

To a stirred solution 4-([1,1′-biphenyl]-4-yloxy)-2-bromo-5-nitrophenol (1.00 g, 2.58 mmol, 1.00 equiv) and Trimethyl(prop-2-yn-1-yloxy) silane (660 mg, 5.17 mmol, 2.00 equiv) in DMF (20.0 mL) were added Pd(PPh₃)₂Cl₂ (180 mg, 0.259 mmol, 0.10 equiv) and CuI (50 mg, 0.25 mmol, 0.10 equiv) and Et₃N (520 mg, 5.17 mmol, 2.00 equiv) at room temperature. The resulting mixture was stirred for 2 h at 80° C. under nitrogen atmosphere. The reaction was quenched by the addition of water (100 mL). The resulting mixture was extracted with EtOAc (3×100 mL). The combined organic layers were washed with brine (2×50 mL), dried over anhydrous na₂SO₄. After filtration, the filtrate was concentrated under reduced pressure. The residue was purified by silica gel column chromatography, eluted with PE/EA (3:1) to afford (5-([1,1′-biphenyl]-4-yloxy)-6-nitrobenzofuran-2-yl) methanol (480 mg, 51.3%) as a brown solid. LC-MS (ESI): m/z 359.90 [M−H]⁻

5-([1,1′-biphenyl]-4-yloxy)-6-nitrobenzofuran-2-yl)methyl di((2-bromoethyl)amino)phosphinate

To a stirred solution of (5-([1,1′-biphenyl]-4-yloxy)-6-nitrobenzofuran-2-yl) methanol (380 mg, 1.05 mmol, 1.00 equiv) in THF (50.0 mL) was added LiHMDS (2.63 mL, 2.63 mmol, 2.50 equiv, 1.0 M in THF) at −78° C. under nitrogen atmosphere. The resulting mixture was stirred for 30 min at −78° C. To the above mixture was added POCl₃ (403 mg, 2.63 mmol, 2.50 equiv). The resulting mixture was stirred for additional 30 min at −78° C. Then to the above mixture was added 2-bromoethanamine hydrobromide (1.72 g, 8.41 mmol, 8.00 equiv) and DIEA (1.36 g, 10.5 mmol, 10.0 equiv). The resulting mixture was stirred for additional 2 h at room temperature. The reaction was quenched with water at room temperature. The resulting mixture was extracted with EtOAc (3×50 mL). The combined organic layers were washed with brine (2×30 mL), dried over anhydrous na₂SO₄. After filtration, the filtrate was concentrated under reduced pressure. The residue was purified by silica gel column chromatography, eluted with PE/EA (1:8) to afford (5-([1,1′-biphenyl]-4-yloxy)-6-nitrobenzofuran-2-yl)methyl di((2-bromoethyl)amino)phosphinate (310 mg, 45.1%) as a yellow solid. LC-MS (ESI): m/z 649.95, 652.00, 653.95 [M−H]⁻

5-([1,1′-biphenyl]-4-yloxy)-6-nitrobenzofuran-2-yl)methyl di(aziridin-1-yl)phosphinate

To a stirred solution (5-([1,1′-biphenyl]-4-yloxy)-6-nitrobenzofuran-2-yl)methyl di((2-bromoethyl)amino)phosphinate (260 mg, 0.40 mmol, 1.00 equiv) and Ag₂O (461 mg, 1.99 mmol, 5.00 equiv) in THF (10.0 mL) was added DIEA (257 mg, 1.99 mmol, 5.00 equiv) at room temperature. The resulting mixture was stirred for 1 h at 70° C. The resulting mixture was filtered, the filter cake was washed with EtOAc (3×20 mL). The resulting mixture was diluted with water, and extracted with EtOAc (3×50 mL). The combined organic layers were washed with brine (2×30 mL), dried over anhydrous na₂SO₄. After filtration, the filtrate was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 44% B to 64% B in 9 min, 64% B; Wave Length: 254/220 nm; RT1 (min): 10.25) to afford (5-([1,1′-biphenyl]-4-yloxy)-6-nitrobenzofuran-2-yl)methyl di(aziridin-1-yl)phosphinate (123.7 mg, 63.2%) as a white solid. LC-MS (ESI): m/z 492.15 [M+H]⁺, ¹H NMR (300 MHz, DMSO-d₆) δ 8.55 (d, J=0.9 Hz, 1H), 7.74-7.58 (m, 5H), 7.47 (t, J=7.5 Hz, 2H), 7.36 (t, J=7.3 Hz, 1H), 7.20-7.08 (m, 3H), 5.27 (d, J=8.3 Hz, 2H), 2.23-2.00 (m, 8H). ³¹P NMR (121 MHz, DMSO-d₆) δ 31.12.

### Illustration 35. Synthesis of (R)-1-(5-(3-(dimethylcarbamoyl)phenoxy)-6-nitrobenzofuran-2-yl)ethyl di(aziridin-1-yl)phosphinate & (S)-1-(5-(3-(dimethylcarbamoyl)phenoxy)-6-nitrobenzofuran-2-yl)ethyl di(aziridin-1-yl)phosphinate (232&233)

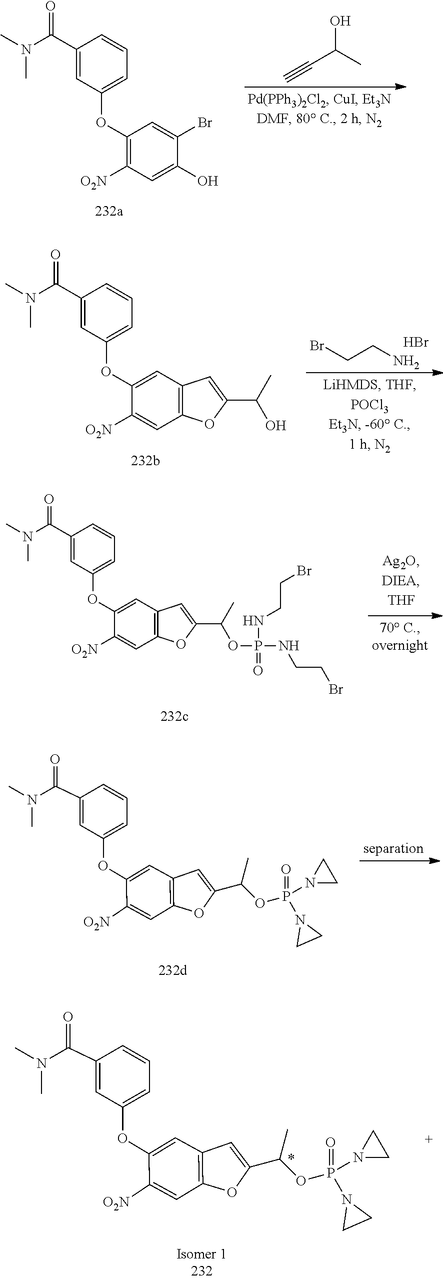

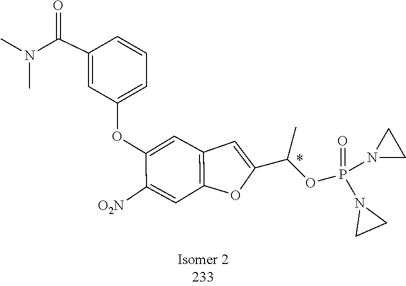 [SMILES: CC(OP(=O)(N1CC1)N1CC1)c1cc2cc(Oc3cccc(C(=O)N(C)C)c3)c([N+](=O)[O-])cc2o1]

3-((2-(1-hydroxyethyl)-6-nitrobenzofuran-5-yl)oxy)-N,N-dimethylbenzamide

To a stirred solution of were added 3-(5-bromo-4-hydroxy-2-nitrophenoxy)-N,N-dimethylbenzamide (1.60 g, 4.20 mmol, 1.00 equiv) and 3-butyn-2-ol (588 mg, 8.40 mmol, 2.00 equiv) in DMF (60.0 mL) were added Pd(PPh₃)₂Cl₂ (295 mg, 0.42 mmol, 0.10 equiv), CuI (79.9 mg, 0.420 mmol, 0.10 equiv), Et₃N (849 mg, 8.40 mmol, 2.00 equiv) at room temperature. The resulting mixture was stirred for 2 h at 80° C. under nitrogen atmosphere. The reaction was quenched with Water at room temperature. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by reversed-phase flash chromatography with the following conditions: column, C₁₈; Mobile Phase A: Water (10 mmol/L NH₄HCO₃), Mobile Phase B: ACN; Flow rate: 60 mL/min; Gradient: 10% B to 95% B in 30 min, 35% B; Wave Length: 220 nm. This resulted in 3-((2-(1-hydroxyethyl)-6-nitrobenzofuran-5-yl)oxy)-N,N-dimethylbenzamide (963 mg, 61.9%) as a brown oil. LC-MS (ESI): m/z 371.12 [M+H]⁺

1-(5-(3-(dimethylcarbamoyl)phenoxy)-6-nitrobenzofuran-2-yl)ethyl di((2-bromoethyl)amino)phosphinate

To a stirred mixture of 3-((2-(1-hydroxyethyl)-6-nitrobenzofuran-5-yl)oxy)-N,N-dimethylbenzamide (713 mg, 1.92 mmol, 1.00 equiv) in THF (128 mL) was added LiHMDS (4.81 mL, 4.81 mmol, 2.50 equiv, 1.0 mol/L in THF) dropwise at −60° C. under nitrogen atmosphere. The resulting mixture was stirred for 0.5 h at −60° C. under nitrogen atmosphere. To the above mixture was added POCl₃ (738 mg, 4.81 mmol, 2.50 equiv) dropwise at −60° C. The resulting mixture was stirred for additional 1 h at −60° C. To the above mixture was added 2-bromoethanamine hydrobromide (2.37 g, 11.5 mmol, 6.00 equiv) and Et₃N (1.56 g, 15.4 mmol, 8.00 equiv) dropwise at −60° C. under nitrogen atmosphere. The resulting mixture was stirred for additional 1 h at room temperature. The reaction was quenched by the addition of water (150 mL) and extracted with EtOAc (3×80 mL). The combined organic layers were concentrated under reduced pressure. The residue was purified by reversed-phase flash chromatography with the following conditions: column, C₁₈; Mobile Phase A: Water (10 mmol/L NH₄HCO₃), Mobile Phase B: ACN; Flow rate: 70 mL/min; Gradient: 10% B to 90% B in 35 min, 45% B; Wave Length: 220 nm) to afford 1-(5-(3-(dimethylcarbamoyl)phenoxy)-6-nitrobenzofuran-2-yl)ethyl di((2-bromoethyl)amino)phosphinate (405 mg, 31.8%) as a brown oil. LC-MS (ESI): m/z 660.90, 662.75, 664.90 [M+H]⁺

1-(5-(3-(dimethylcarbamoyl)phenoxy)-6-nitrobenzofuran-2-yl)ethyl di(aziridin-1-yl)phosphinate

To a stirred mixture of 1-(5-(3-(dimethylcarbamoyl)phenoxy)-6-nitrobenzofuran-2-yl)ethyl di((2-bromoethyl)amino)phosphinate (395 mg, 0.60 mmol, 1.00 equiv) in THF (20.0 mL) were added Ag₂O (691 mg, 2.98 mmol, 5.00 equiv) and DIEA (385 mg, 2.98 mmol, 5.00 equiv) at room temperature. The resulting mixture was stirred for overnight at 70° C. The resulting mixture was filtered; the filter cake was washed with EtOAc (3×10 mL). The filtrate was concentrated under reduced pressure. The resulting mixture was diluted with water (50 mL), and extracted with EtOAc (3×20 mL). The combined organic layers were washed with brine (3×10 mL), dried over anhydrous Na₂SO₄. After filtration, the filtrate was concentrated under reduced pressure. The crude product was purified by Prep-HPLC with the following conditions: (Column: Ultimate AQ-C18 Column, 50*250 mm, 10 μm; Mobile Phase A: Water (10 mmol/L NH₄HCO₃), Mobile Phase B: ACN; Flow rate: 100 mL/min; Gradient: 40% B to 70% B in 20 min, 60% B; Wave Length: 220 nm. This resulted in 1-(5-(3-(dimethylcarbamoyl)phenoxy)-6-nitrobenzofuran-2-yl)ethyl di(aziridin-1-yl)phosphinate (194 mg, 65.0%) as a yellow oil. LC-MS (ESI): m/z 501.15 [M+H]⁺

(R)-1-(5-(3-(dimethylcarbamoyl)phenoxy)-6-nitrobenzofuran-2-yl)ethyl di(aziridin-1-yl)phosphinate & (S)-1-(5-(3-(dimethylcarbamoyl)phenoxy)-6-nitrobenzofuran-2-yl)ethyl di(aziridin-1-yl)phosphinate

The 1-(5-(3-(dimethylcarbamoyl)phenoxy)-6-nitrobenzofuran-2-yl)ethyl di(aziridin-1-yl)phosphinate (190 mg, 0.38 mmol, 1.00 equiv) was separated by Prep-Chiral HPLC with the following conditions (Column: CHIRAL ART Cellulose-SC, 2*25 cm, 5 μm; Mobile Phase A: Hex: MtBE=1: 1 (0.5% 2M NH₃—MEOH), Mobile Phase B: MeOH; Flow rate: 20 mL/min; Gradient: 50% B to 50% B in 20 min; Wave Length: 210/240 nm; RTI (min): 13.85; RT2 (min): 15.85; Sample Solvent: MeOH: DCM=1:1; InJection Volume: 0.6 mL; Number Of Runs: 7.

Isomer 1 (232), 34.8 mg, 18.3%, ee>99%, LC-MS (ESI): m/z 500.95 [M+H]^{+ 1}H NMR (400 MHz, DMSO-d₆) δ 8.53 (d, J=1.0 Hz, 1H), 7.61 (d, J=1.4 Hz, 1H), 7.45 (t, J=7.9 Hz, 1H), 7.19-7.13 (m, 1H), 7.13-7.07 (m, 2H), 7.01-6.94 (m, 1H), 5.78-5.69 (m, 1H), 2.93 (s, 3H), 2.87 (s, 3H), 2.17-2.03 (m, 8H), 1.67 (d, J=6.6 Hz, 3H). ³¹P NMR (162 MHz, DMSO-d₆) δ 30.48 (s).

Isomer 2 (233), 29.0 mg, 15.3%, ee>99%, LC-MS (ESI): m/z 501.00 [M+H]^{+ 1}H NMR (400 MHz, DMSO-d₆) δ 8.53 (s, 1H), 7.61 (s, 1H), 7.49-7.40 (m, 1H), 7.19-7.11 (m, 1H), 7.13-7.06 (m, 2H), 7.01-6.94 (m, 1H), 5.78-5.67 (m, 1H), 2.94 (s, 3H), 2.87 (s, 3H), 2.18-2.01 (m, 8H), 1.67 (d, J=6.6 Hz, 3H). ³¹P NMR (162 MHz, DMSO-d₆) δ 30.48 (s).

Compounds of the present disclosure can be generally prepared following similar methods as described hereinabove. The tables below show additional exemplified examples and characterizations thereof.

TABLE 1A
Characterization of compounds (see General Procedure 1)

| Compound No. | Structure | LCMS; ¹H NMR (ppm); Retention time |
|---|---|---|
| 1 | 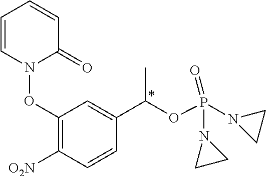 [SMILES: CC(OP(=O)(N1CC1)N1CC1)c1ccc([N+](=O)[O-])c(On2ccccc2=O)c1] | LCMS (ESI): m/z 405.2 [M + H]⁺; ¹H NMR (400 MHz, CDCl₃) δ 8.12 (d, J = 8.5 Hz, 1H), 7.54-7.31 (m, 3H), 7.35-7.32 (m, 1H), 6.65-6.61 (m, 1H), 6.27-6.24 (m, 1H), 5.62-5.57 (m, 1H), 5.53-5.47 (m, 2H), 2.22-1.94 (m, 8H), 1.56 (d, J = 6.6 Hz, 3H). ³¹P NMR (162 MHz, CDCl₃) δ 29.90 (s), |
| 2 | 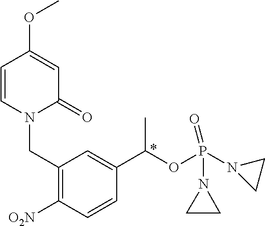 [SMILES: COc1ccn(Cc2cc(C(C)OP(=O)(N3CC3)N3CC3)ccc2[N+](=O)[O-])c(=O)c1] | LCMS (ESI): m/z 435.2 [M + H]⁺; ¹H NMR (400 MHz, CDCl₃) δ 8.12 (d, J = 8.5 Hz, 1H), 7.49 (dd, J = 8.5, 1.7 Hz, 1H), 7.18 (d, J = 7.6 Hz, 1H), 7.08 (s, 1H), 6.01-5.94 (m, 2H), 5.65-5.55 (m, 1H), 5.46 (d, J = 1.9 Hz, 2H), 2.21-1.98 (m, 8H), 1.56 (d, J = 6.5 Hz, 3H). ³¹P NMR (162 MHz, CDCl₃) δ 29.89 (s). |
| 3 | 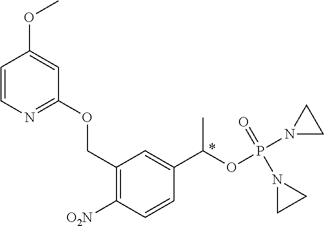 [SMILES: COc1ccnc(OCc2cc(C(C)OP(=O)(N3CC3)N3CC3)ccc2[N+](=O)[O-])c1] | LCMS (ESI): m/z 435.2 [M + H]⁺; ¹H NMR (400 MHz, CDCl₃) δ 8.19-8.08 (m, 1H), 7.97-7.87 (m, 1H), 7.76-7.67 (m, 1H), 7.54-7.41 (m, 1H), 6.57-6.44 (m, 1H), 6.41-6.27 (m, 1H), 5.87-5.73 (m, 2H), 5.72-5.47 (m, 1H), 3.85 (s, 3H), 2.23-1.90 (m, 8H), 1.60 (d, J = 6.4 Hz, 3H). ³¹P NMR (162 MHz, CDCl₃) δ 30.24 (s). |
| 4 | 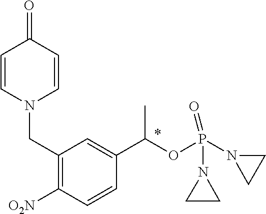 [SMILES: CC(OP(=O)(N1CC1)N1CC1)c1ccc([N+](=O)[O-])c(Cn2ccc(=O)cc2)c1] | LCMS (ESI): m/z 405.2 [M + H]⁺; ¹H NMR (400 MHz, CDCl) δ 8.22 (m, 1H), 7.63-7.51 (m, 1H), 7.35- 7.30 (m, 2H), 7.07 (s, 1H), 6.46 (m, 2H), 5.71-5.61 (m, 1H), 5.40 (m, 2H), 2.26-1.95 (m, 8H), 1.59 (d, J = 6.6 Hz, 3H). ³¹P NMR (162 MHz, CDCl₃) δ 30.02 (s). |
| 5 | 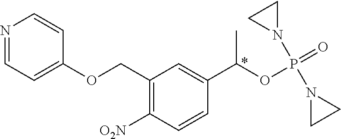 [SMILES: CC(OP(=O)(N1CC1)N1CC1)c1ccc([N+](=O)[O-])c(COc2ccncc2)c1] | LCMS (ESI): m/z 405 [M + H]⁺; ¹H NMR (400 MHz, CDCl₃) δ 8.49 (dd, J = 4.8, 1.6 Hz, 2H), 8.24-8.17 (m, 1H), 7.87-7.81 (m, 1H), 7.59-7.52 (m, 1H), 6.96-6.92 (m, 2H), 5.78- 5.66 (m, 1H), 5.55 (s, 2H), 2.27-1.98 (m, 8H), 1.65-1.63 (m, 3H). ³¹P NMR (162 MHz, CDCl₃) δ 30.33 (s). |
| 6 | 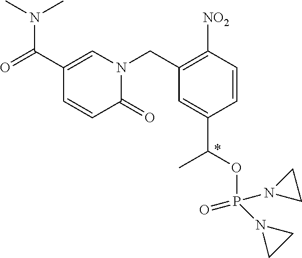 [SMILES: CC(OP(=O)(N1CC1)N1CC1)c1ccc([N+](=O)[O-])c(Cn2cc(C(=O)N(C)C)ccc2=O)c1] | LCMS (ESI): m/z 476.1 [M + H]⁺; ¹H NMR (400 MHz, DMSO-d₆) δ 8.15 (d, J = 2.4 Hz, 1H), 8.12 (d, J = 8.5 Hz, 1H), 7.63 (dd, J = 9.4, 2.5 Hz, 1H), 7.57 (dd, J = 8.5, 1.5 Hz, 1H), 7.06 (d, J = 1.1 Hz, 1H), 6.49 (d, J = 9.4 Hz, 1H), 5.63-5.53 (m, 1H), 5.52-5.40 (m, 2H), 2.98 (s, 6H), 2.07-1.81 (m, 8H), 1.43 (d, J = 6.5 Hz, 3H). ³¹P NMR (162 MHz, DMSO-d₆) δ 29.55 (s). |
| 7 | 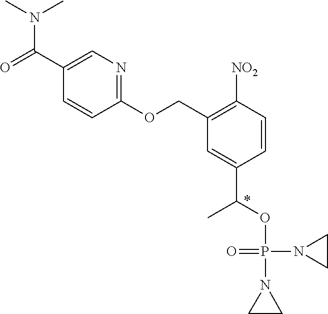 [SMILES: CC(OP(=O)(N1CC1)N1CC1)c1ccc([N+](=O)[O-])c(COc2ccc(C(=O)N(C)C)cn2)c1] | LCMS (ESI): m/z 476.2 [M + H]⁺; ¹H NMR (400 MHz, CDCl₃) δ 8.23 (d, J = 1.8 Hz, 1H), 8.13 (d, J = 8.5 Hz, 1H), 7.79-7.71 (m, 2H), 7.50 (dd, J = 8.5, 1.6 Hz, 1H), 6.90 (d, J = 8.5 Hz, 1H), 5.84 (s, 2H), 5.74-5.63 (m, 1H), 3.08 (s, 6H), 2.20-2.01 (m, 8H), 1.61 (d, J = 6.6 Hz, 3H). ³¹P NMR (162 MHz, CDCl₃) δ 30.29 (s). |

TABLE 1B
Characterization of compounds (see General Procedure 2)

| Compound | | |
|---|---|---|
| No. | Structure | LCMS; ¹H NMR (ppm); Retention time |
| 8 | 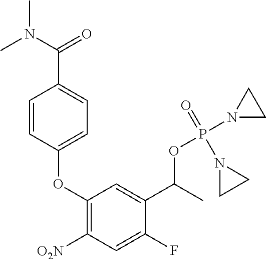 [SMILES: CC(OP(=O)(N1CC1)N1CC1)c1cc(Oc2ccc(C(=O)N(C)C)cc2)c([N+](=O)[O-])cc1F] | LCMS (ESI): m/z 479.1 [M + H]⁺; ¹H NMR (400 MHz, CDCl₃) δ 7.72 (d, J = 9.0 Hz, 1H), 7.43 (d, J = 8.6 Hz, 2H), 7.29 (d, J = 6.0 Hz, 1H), 6.98 (d, J = 8.6 Hz, 2H), 5.92-5.81 (m, 1H), 3.07 (s, 3H), 3.00 (s, 3H), 2.15-2.00 (m, 8H), 1.58 (d, J = 6.5 Hz, 3H). ³¹P NMR (162 MHz, CDCl₃) δ 30.25 (s). |
| 9 | 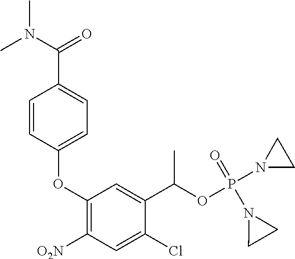 [SMILES: CC(OP(=O)(N1CC1)N1CC1)c1cc(Oc2ccc(C(=O)N(C)C)cc2)c([N+](=O)[O-])cc1Cl] | LCMS (ESI): m/z 495.1 [M + H]⁺; ¹H NMR (400 MHz, CDCl₃) δ 8.01 (s, 1H), 7.51-7.43 (m, 2H), 7.35 (s, 1H), 7.09-7.01 (m, 2H), 5.98-5.88 (m, 1H), 3.11 (s, 3H), 3.03 (s, 3H), 2.18-1.98 (m, 8H), 1.58 (d, J = 6.5 Hz, 3H). ³¹P NMR (162 MHz, CDCl₃) δ 30.05 (s). |

TABLE 1C
Characterization of compounds (see General Procedure 4)

| Compound | | |
|---|---|---|
| No. | Structure | LCMS; ¹H NMR (ppm); Retention time |
| 10 | 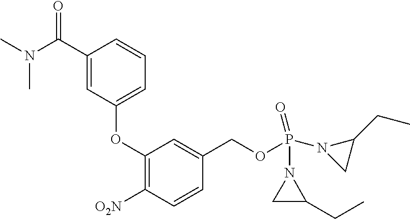 [SMILES: CCC1CN1P(=O)(OCc1ccc([N+](=O)[O-])c(Oc2cccc(C(=O)N(C)C)c2)c1)N1CC1CC] | LCMS (ESI): m/z 503.2 [M + H]⁺; ¹H NMR (400 MHz, CDCl₃) δ 8.08-7.87 (m, 1H), 7.50-7.37 (m, 1H), 7.33-7.29 (m, 1H), 7.26-7.21 (m, 1H), 7.14-7.02 (m, 3H), 5.20-5.08 (m, 2H), 3.10 (s, 3H), 2.99 (s, 3H) 2.55-2.42 (m, 2H), 2.37-2.24 (m, 2H), 1.99-1.80 (m, 2H), 1.61-1.40 (m, 4H), 1.03- 0.93 (m, 6H) ³¹P NMR (162 MHz, CDCl₃) δ 36.10-23.43 (m) |
| 11 | 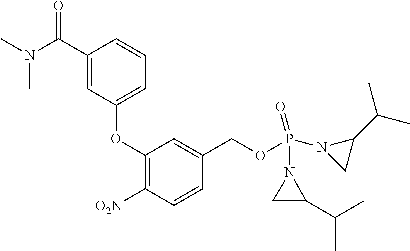 [SMILES: CC(C)C1CN1P(=O)(OCc1ccc([N+](=O)[O-])c(Oc2cccc(C(=O)N(C)C)c2)c1)N1CC1C(C)C] | LCMS (ESI): m/z 531.3 [M + H]⁺; ¹H NMR (400 MHz, CDCl₃) δ 7.94-7.88 (m, 1H), 7.37-7.30 (m, 1H), 7.24-7.21 (m, 1H), 7.17-7.12 (m, 1H), 7.08-6.91 (m, 3H), 5.17-4.93 (m, 2H), 3.02 (s, 3H), 2.90 (s, 3H), 2.32-2.13 (m, 4H), 1.90-1.73 (m, 2H), 1.46-1.26 (m, 2H), 0.98-0.82 (m, 12H). ³¹P NMR (162 MHz, CDCl₃) δ 30.86 (s), 30.38 (s), 28.90 (s). |

TABLE 1D
Characterization of compounds (see General Procedure 5)

| Compound | | |
|---|---|---|
| No. | Structure | LCMS; ¹H NMR (ppm); Retention time |
| 12 | 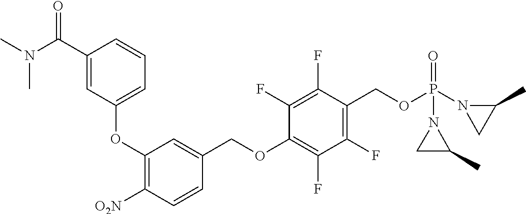 | LCMS (ESI): m/z 653.2 [M + H]⁺; ¹H NMR (400 MHz, CDCl₃) δ 8.01 (d, J = 8.4 Hz, 1H), 7.48-7.44 (m, 1H), 7.34-7.31 (m, 1H), 7.28-7.25 (m, 1H), 7.15-7.09 (m, 3H), 5.25 (s, 2H), 5.21 (d, J = 5.8 Hz, 2H), 3.11 (s, 3H), 3.00 (s, 3H), 2.64-2.52 (m, 2H), 2.43- 2.33 (m, 2H), 1.96-1.85 (m, 2H), 1.30-1.28 (m, 6H). ³¹P NMR (162 MHz, CDCl₃) δ 29.22 (s) ¹⁹F NMR (376 MHz, CDCl₃) δ −143.13- −143.20 (m), −156.15- −156.24 (m). |
| 13 | 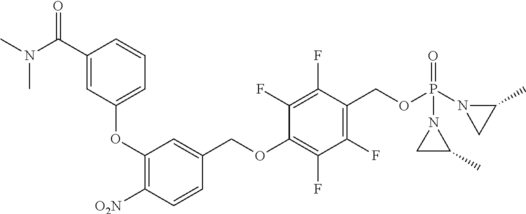 | LCMS (ESI): m/z 653.2 [M + H]⁺; ¹H NMR (400 MHz, CDCl₃) δ 8.01 (d, J = 8.4 Hz, 1H), 7.48-7.43 (m, 1H), 7.33-7.31 (m, 1H), 7.28-7.26 (m, 1H), 7.14-7.09 (m, 3H), 5.25 (s, 2H), 5.21 (d, J = 5.8 Hz, 2H), 3.11 (s, 3H), 2.99 (s, 3H), 2.65-2.50 (m, 2H), 2.43- 2.33 (m, 2H), 1.95-1.85 (m, 2H), 1.30-1.27 (m, 6H). ³¹P NMR (162 MHz, CDCl₃) δ 29.22 (s). ¹⁹F NMR (376 MHz, CDCl₃) δ −143.13- −143.21 (m), −156.15- −156.24 (m) |
| 14 | 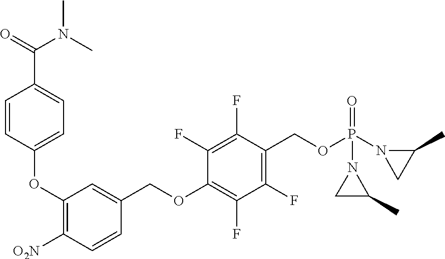 [SMILES: C[C@H]1CN1P(=O)(OCc1c(F)c(F)c(OCc2ccc([N+](=O)[O-])c(Oc3ccc(C(=O)N(C)C)cc3)c2)c(F)c1F)N1C[C@@H]1C] | LCMS (ESI): m/z 653.2 [M + H]⁺; ¹H NMR (400 MHz, CDCl₃) δ 7.98 (d, J = 8.4 Hz, 1H), 7.47 (d, J = 8.4 Hz, 2H), 7.28 (d, J = 8.3 Hz, 1H), 7.12 (s, 1H), 7.05 (d, J = 8.4 Hz, 2H), 5.27-5.09 (m, 4H), 3.10 (s, 3H), 3.02 (s, 3H), 2.62-2.47 (m, 2H), 2.43-2.26 (m, 2H), 1.94-1.80 (m, 2H), 1.32-1.21 (m, 6H). ³¹P NMR (162 MHz, CDCl₃) δ 29.22 (s) ¹⁹F NMR (376 MHz, CDCl₃) δ −141.37- −143.29 (m), −156.04-−156.42 (m) |
| 15 | 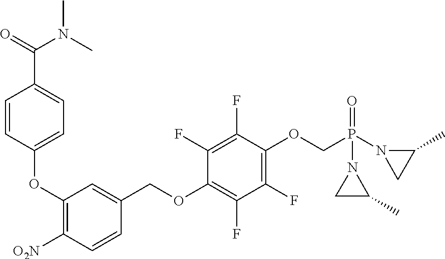 [SMILES: C[C@@H]1CN1P(=O)(COc1c(F)c(F)c(OCc2ccc([N+](=O)[O-])c(Oc3ccc(C(=O)N(C)C)cc3)c2)c(F)c1F)N1C[C@H]1C] | LCMS (ESI): m/z 653.2 [M + H]⁺; ¹H NMR (400 MHz, CDCl₃) δ 8.02 (d, J = 8.4 Hz, 1H), 7.50 (d, J = 8.3 Hz, 2H), 7.32 (d, J = 8.3 Hz, 1H), 7.15 (s, 1H), 7.09 (d, J = 8.3 Hz, 2H), 5.33-5.03 (m, 4H), 3.13 (s, 3H), 3.05 (s, 3H), 2.65-2.51 (m, 2H), 2.45-2.30 (m, 2H), 1.98-1.83 (m, 2H), 1.33-1.25 (m, 6H). ³¹P NMR (162 MHz, CDCl₃) δ 29.23 (s). ¹⁹F NMR (376 MHz, CDCl₃) δ −142.91- −143.35 (m), −156.03-−156.39 (m). |

TABLE 2
Characterization of compounds (see General Procedure 8)

| Com- | | |
|---|---|---|
| pound | | |
| No. | Structure | LCMS; ¹H NMR (ppm); Retention time |
| 16 | 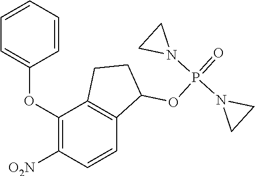 [SMILES: O=[N+]([O-])c1ccc2c(c1Oc1ccccc1)CCC2OP(=O)(N1CC1)N1CC1] | LCMS (ESI): m/z 402.2 [M + H]⁺; ¹H NMR (400 MHz, CDCl₃) δ 7.88 (d, J = 8.2 Hz, 1H), 7.50 (d, J = 8.2 Hz, 1H), 7.35-7.28 (m, 2H), 7.08 (t, J = 7.4 Hz, 1H), 6.87 (d, J = 7.8 Hz, 2H), 6.05-5.94 (m, 1H), 2.95-2.76 (m, 1H), 2.66-2.45 (m, 2H), 2.34-2.13 (m, 9H). ³¹P NMR (162 MHz, CDCl₃) δ 29.97 (s). |
| 17 | 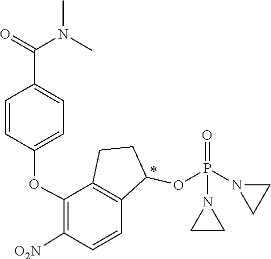 [SMILES: CN(C)C(=O)c1ccc(Oc2c([N+](=O)[O-])ccc3c2CCC3OP(=O)(N2CC2)N2CC2)cc1] | LCMS (ESI): m/z 473.2 [M + H]⁺; ¹H NMR (400 MHz, CDCl₃) δ 7.91 (d, J = 8.2 Hz, 1H), 7.54 (d, J = 8.2 Hz, 1H), 7.40 (d, J = 8.6 Hz, 2H), 6.88 (d, J = 8.7 Hz, 2H), 6.06-5.94 (m, 1H), 3.09 (s, 3H), 3.03 (s, 3H), 2.91-2.78 (m, 1H), 2.67-2.49 (m, 2H), 2.35-2.13 (m, 9H). ³¹P NMR (162 MHz, CDCl₃) δ 29.99 (s). ee: >99% Retention time: 3.963 min; Column: ChiralPak AD, 250 × 4.6 mm I.D., 5 μm, Mobile phase: A for CO₂ and B for ethanol (0.05% DEA), Gradient: 8 min @ B 40%, Flow rate: 2.0 mL/min, Back pressure: 100 bar, Column temperature: 35° C. |
| 18 | 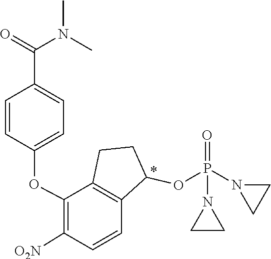 [SMILES: CN(C)C(=O)c1ccc(Oc2c([N+](=O)[O-])ccc3c2CCC3OP(=O)(N2CC2)N2CC2)cc1] | LCMS (ESI): m/z 473.2 [M + H]⁺; ¹H NMR (400 MHz, CDCl₃) δ 7.91 (d, J = 8.2 Hz, 1H), 7.54 (d, J = 8.2 Hz, 1H), 7.40 (d, J = 8.7 Hz, 2H), 6.88 (d, J = 8.7 Hz, 2H), 6.09-5.93 (m, 1H), 3.09 (s, 3H), 3.03 (s, 3H), 2.90-2.79 (m, 1H), 2.67-2.51 (m, 2H), 2.36-2.09 (m, 9H). ³¹P NMR (162 MHz, CDCl₃) δ 29.99 (s). ee: >99% Retention time: 6.352 min; Column: ChiralPak AD, 250 × 4.6 mm I.D., 5 μm, Mobile phase: A for CO₂ and B for ethanol (0.05% DEA), Gradient: 8 min @ B 40%, Flow rate: 2.0 mL/min, Back pressure: 100 bar, Column temperature: 35° C. |
| 19 | 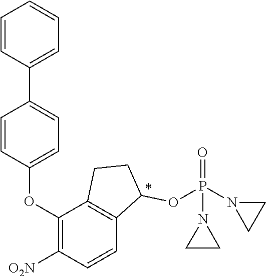 [SMILES: O=[N+]([O-])c1ccc2c(c1Oc1ccc(-c3ccccc3)cc1)CCC2OP(=O)(N1CC1)N1CC1] | LCMS (ESI): m/z 500.2 [M + Na]⁺; ¹H NMR (400 MHz, CDCl₃) δ 7.90 (d, J = 8.0 Hz, 1H), 7.62-7.47 (m, 5H), 7.47-7.38 (m, 2H), 7.37-7.29 (m, 1H), 6.94 (d, J = 7.7 Hz, 2H), 6.10-5.94 (m, 1H), 2.97-2.84 (m, 1H), 2.73-2.51 (m, 2H), 2.37-2.12 (m, 9H). ³¹P NMR (162 MHz, CDCl₃) δ 29.99 (s) ee: >99% Retention time: 4.803 min; Column: ChiralPak IH, 100 × 4.6 mm I.D., 5 μm; Mobile phase: A for CO₂ and B for ethanol (0.05% DEA); Gradient: 8 min @ 20% B; Flow rate: 2.5 mL/min; Column temperature: 40° C. |
| 20 | 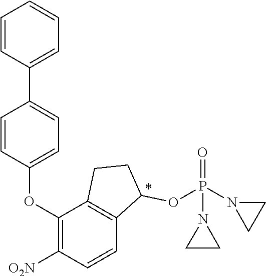 [SMILES: O=[N+]([O-])c1ccc2c(c1Oc1ccc(-c3ccccc3)cc1)CCC2OP(=O)(N1CC1)N1CC1] | LCMS (ESI): m/z 500.2 [M + Na]⁺; ¹H NMR (400 MHz, CDCl₃) δ 7.90 (d, J = 8.0 Hz, 1H), 7.59-7.47 (m, 5H), 7.46-7.37 (m, 2H), 7.36-7.28 (m, 1H), 6.94 (d, J = 7.9 Hz, 2H), 6.09-5.93 (m, 1H), 3.01-2.80 (m, 1H), 2.77-2.49 (m, 2H), 2.39-2.03 (m, 9H). ³¹P NMR (162 MHz, CDCl₃) δ 2.99 (s). ee: 99% Retention time: 5.694 min; Column: ChiralPak IH, 100 × 4.6 mm I.D., 5 μm; Mobile phase: A for CO₂ and B for ethanol (0.05% DEA); Gradient: 8 min @ 20% B; Flow rate: 2.5 mL/min; Column temperature: 40° C. |
| 21 | 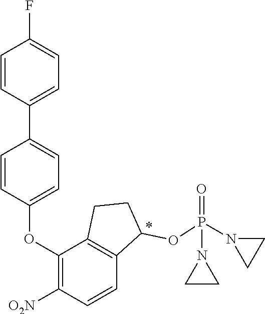 [SMILES: O=[N+]([O-])c1ccc2c(c1Oc1ccc(-c3ccc(F)cc3)cc1)CCC2OP(=O)(N1CC1)N1CC1] | LCMS (ESI): m/z 496.1 [M + H]⁺; ¹H NMR (400 MHz, CDCl₃) δ 7.90 (d, J = 8.2 Hz, 1H), 7.54-7.45 (m, 5H), 7.13-7.07 (m, 2H), 6.95-6.90 (m, 2H), 6.05-5.95 (m, 1H), 3.65- 3.54 (m, 1H), 2.95-2.85 (m, 1H), 2.67-2.53 (m, 2H), 2.35-2.10 (m, 9H), ³¹P NMR (162 MHz, CDCl₃) δ 29.97 (s). ¹⁹F NMR (376 MHz, CDCl₃) δ −114.78-−117.37 (m) ee: >99% Retention time: 1.917 min; Column: ChiralPak IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 22 | 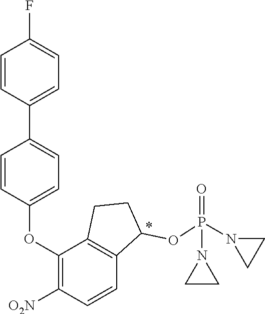 [SMILES: O=[N+]([O-])c1ccc2c(c1Oc1ccc(-c3ccc(F)cc3)cc1)CCC2OP(=O)(N1CC1)N1CC1] | LCMS (ESI): m/z 496.2 [M + H]⁺; ¹H NMR (400 MHz, CDCl₃) δ 7.90 (d, J = 8.2 Hz 1H), 7.54-7.44 (m, 5H), 7.14-7.07 (m, 2H), 6.95-6.89 (m, 2H), 6.09-5.94 (m, 1H), 2.96- 2.86 (m, 1H), 2.69-2.51 (m, 2H), 2.36-2.12 (m 9H). ³¹P NMR (162 MHz, CDCl₃) δ 30.00 (s). ¹⁹F NMR (376 MHz, CDCl₃) δ −115.94 (s). ee: 96% Retention time: 2.321 min; Column: ChiralPak IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 23 | 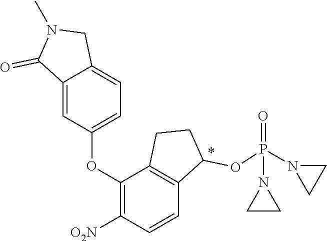 [SMILES: CN1Cc2ccc(Oc3c([N+](=O)[O-])ccc4c3CCC4OP(=O)(N3CC3)N3CC3)cc2C1=O] | LCMS (ESI): m/z 471.1 [M + H]⁺; ¹H NMR (400 MHz, CDCl₃) δ 7.93 (d, J = 8.2 Hz, 1H), 7.55 (d, J = 8.2 Hz, 1H), 7.41 (d, J = 8.2 Hz, 1H), 7.22 (dd, J = 8.2, 2.4 Hz, 1H), 7.13 (d, J = 2.3 Hz, 1H), 6.04-5.97 (m, 1H), 4.35 (s, 2H), 3.18 (s, 3H), 2.91- 2.79 (m, 1H), 2.66-2.51 (m, 2H), 2.29-2.19 (m, 9H). ³¹P NMR (162 MHz, CDCl₃) δ 29.67 (s). ee: >99% Retention time: 5.659 min; Column: ChiralPak IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 24 | 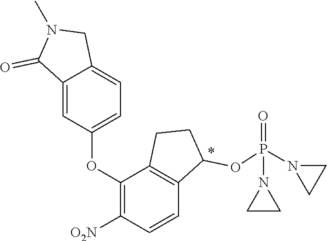 [SMILES: CN1Cc2ccc(Oc3c([N+](=O)[O-])ccc4c3CCC4OP(=O)(N3CC3)N3CC3)cc2C1=O] | LCMS (ESI): m/z 471.1 [M + H]⁺; ¹H NMR (400 MHz, CDCl₃) δ 7.93 (d, J = 8.2 Hz, 1H), 7.55 (d, J = 8.2 Hz, 1H), 7.41 (d, J = 8.3 Hz, 1H), 7.22 (dd, J = 8.2, 2.4 Hz, 1H), 7.13 (d, J = 2.3 Hz, 1H), 6.04-5.96 (m, 1H), 4.35 (s, 2H), 3.18 (s, 3H), 2.92- 2.80 (m, 1H), 2.65-2.51 (m, 2H), 2.32-2.15 (m, 9H). ³¹P NMR (162 MHz, CDCl₃) δ 29.67 (s). ee: >99% Retention time: 7.054 min; Column: ChiralPak, IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 25 | 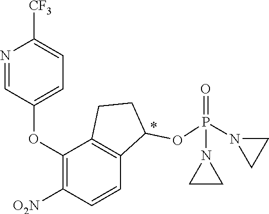 [SMILES: O=[N+]([O-])c1ccc2c(c1Oc1ccc(C(F)(F)F)nc1)CCC2OP(=O)(N1CC1)N1CC1] | LCMS (ESI): m/z 471.1 [M + H]⁺; ¹H NMR (400 MHz, CDCl₃) δ 8.39 (d, J = 2.8 Hz, 1H), 7.99 (d, J = 8.2 Hz, 1H), 7.69-7.60 (m, 2H), 7.26-7.22 (m, 1H), 6.10-5.99 (m, 1H), 2.97-2.85 (m, 1H), 2.73-2.55 (m, 2H), 2.30-2.18 (m, 9H). ³¹P NMR (162 MHz, CDCl₃) δ 30.10 (s). ¹⁹F NMR (376 MHz, CDCl₃) δ −67.04 (s). ee: >99% Retention time: 1.769 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 26 | 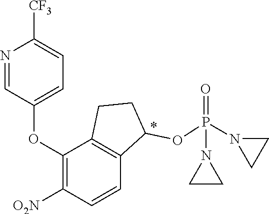 [SMILES: O=[N+]([O-])c1ccc2c(c1Oc1ccc(C(F)(F)F)nc1)CCC2OP(=O)(N1CC1)N1CC1] | LCMS (ESI): m/z 471.1 [M + H]⁺; ¹H NMR (400 MHz, CDCl₃) δ 8.39 (d, J = 2.8 Hz, 1H), 7.99 (d, J = 8.2 Hz, 1H), 7.70-7.61 (m, 2H), 7.26-7.21 (m, 1H), 6.09-5.98 (m, 1H), 2.96-2.86 (m, 1H), 2.72-2.57 (m, 2H), 2.30-2.18 (m, 9H). ³¹P NMR (162 MHz, CDCl₃) δ 30.10 (s). ¹⁹F NMR (376 MHz, CDCl₃) δ −67.04 (s). ee: >99% Retention time: 2.236 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 27 | 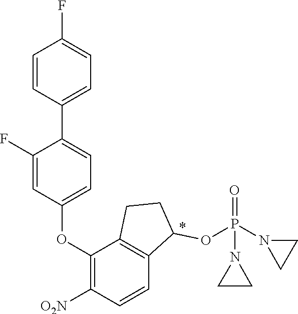 [SMILES: O=[N+]([O-])c1ccc2c(c1Oc1ccc(-c3ccc(F)cc3)c(F)c1)CCC2OP(=O)(N1CC1)N1CC1] | LCMS (ESI): m/z 536.1 [M + Na]⁺; ¹H NMR (400 MHz, CDCl₃) δ 7.93 (d, J = 8.2 Hz, 1H), 7.56 (d, J = 8.2 Hz, 1H), 7.49-7.43 (m, 2H), 7.32 (t, J = 8.6 Hz, 1H), 7.11 (t, J = 8.7 Hz, 2H), 6.76-6.65 (m, 2H), 6.07-5.99 (m, 1H), 3.01-2.90 (m, 1H), 2.75- 2.56 (m, 2H), 2.36-2.13 (m, 9H). ³¹P NMR (162 MHz, CDCl₃) δ 30.02 (s). ¹⁹F NMR (376 MHz, CDCl₃) δ −114.52 (s), −114.69 (s). ee: 99% Retention time: 2.872 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 28 | 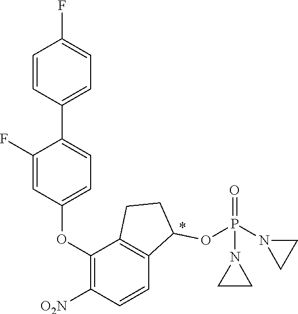 [SMILES: O=[N+]([O-])c1ccc2c(c1Oc1ccc(-c3ccc(F)cc3)c(F)c1)CCC2OP(=O)(N1CC1)N1CC1] | LCMS (ESI): m/z 536.1 [M + Na]⁺; ¹H NMR (400 MHz, CDCl₃) δ 7.93 (d, J = 8.2 Hz, 1H), 7.56 (d, J = 8.2 Hz, 1H), 7.50-7.42 (m, 2H), 7.32 (t, J = 8.6 Hz, 1H), 7.15-7.08 (m, 2H), 6.75-6.66 (m, 2H), 6.06-5.99 (m, 1H), 3.00-2.90 (m, 1H), 2.75-2.56 (m, 2H), 2.34-2.14 (m, 9H). ³¹P NMR (162 MHz, CDCl₃) δ 30.02 (s). ¹⁹F NMR (376 MHz, CDCl₃) δ −114.52 (s), −114.69 (s). ee: 99% Retention time: 4.362 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 29 | 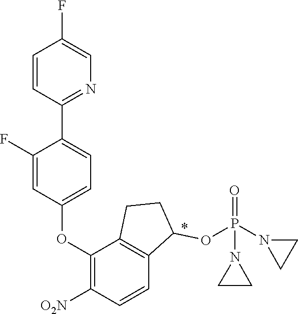 | LCMS (ESI): m/z 515.1 [M + H]⁺; ¹H NMR (400 MHz, CDCl₃) δ 8.72-8.47 (m, 1H), 8.02-7.83 (m, 2H), 7.81-7.73 (m, 1H), 7.62-7.55 (m, 1H), 7.51-7.42 (m, 1H), 6.87-6.63 (m, 2H), 6.12-5.95 (m, 1H), 3.02-2.85 (m, 1H), 2.78-2.52 (m, 2H), 2.36- 2.13 (m, 9H). ³¹P NMR (162 MHz, CDCl₃) δ 29.98 (s). ¹⁹F NMR (376 MHz, CDCl₃) δ −113.78 (s), −128.72 (s). |
| 30 | 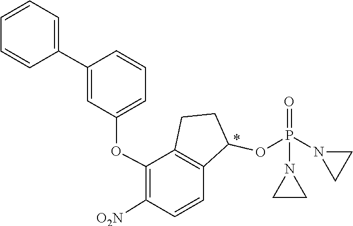 [SMILES: O=[N+]([O-])c1ccc2c(c1Oc1cccc(-c3ccccc3)c1)CCC2OP(=O)(N1CC1)N1CC1] | LCMS (ESI): m/z 478.2 [M + H]⁺; ¹H NMR (400 MHz, CDCl₃) δ 7.82 (d, J = 8.2 Hz, 1H), 7.49-7.41 (m, 3H), 7.39-7.32 (m, 2H), 7.31-7.21 (m, 3H), 7.05-7.01 (m, 1H), 6.79- 6.72 (m, 1H), 5.97-5.88 (m, 1H), 2.88-2.76 (m, 1H), 2.61-2.43 (m, 2H), 2.21-2.08 (m, 9H). ³¹P NMR (162 MHz, CDCl₃) δ 29.90 (s). ee: >99% Retention time: 2.148 min; Column: ChiralPak IB, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 31 | 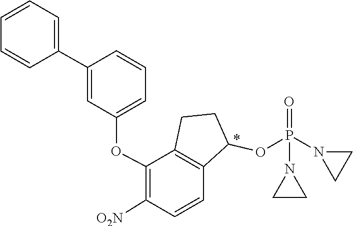 [SMILES: O=[N+]([O-])c1ccc2c(c1Oc1cccc(-c3ccccc3)c1)CCC2OP(=O)(N1CC1)N1CC1] | LCMS (ESI): m/z 478.2 [M + H]⁺; ¹H NMR (400 MHz, CDCl₃) δ 7.82 (d, J = 8.2 Hz, 1H), 7.49-7.41 (m, 3H), 7.39-7.32 (m, 2H), 7.30-7.21 (m, 3H), 7.07-7.01 (m, 1H), 6.78- 6.72 (m, 1H), 5.95-5.88 (m, 1H), 2.87-2.77 (m, 1H), 2.62-2.42 (m, 2H), 2.22-2.08 (m, 8H). ³¹P NMR (162 MHz, CDCl₃) δ 29.90 (s). ee: 99% Retention time: 2.544 min; Column: ChiralPak IB, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 32 | 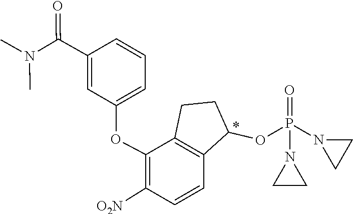 [SMILES: CN(C)C(=O)c1cccc(Oc2c([N+](=O)[O-])ccc3c2CCC3OP(=O)(N2CC2)N2CC2)c1] | LCMS (ESI): m/z 473.25 [M + H]⁺; ¹H NMR (400 MHz, DMSO-d₆) δ 8.04 (d, J = 8.2 Hz, 1H), 7.55 (d, J = 8.2 Hz, 1H), 7.43 (t, J = 7.9 Hz, 1H), 7.13 (dt, J = 7.6, 1.2 Hz, 1H), 7.01 (ddd, J = 8.3, 2.7, 1.0 Hz, 1H), 6.91-6.82 (m, 1H), 5.94 (q, J = 6.7 Hz, 1H), 2.94 (s, 3H), 2.85 (s, 3H), 2.79-2.66 (m, 1H), 2.61-2.52 (m, 2H), 2.20-2.01 (m, 9H). ³¹P NMR (162 MHz, DMSO-d₆) δ 30.10 (d, J = 2.8 Hz). ee: >99% |
| 33 | 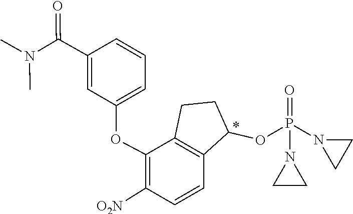 [SMILES: CN(C)C(=O)c1cccc(Oc2c([N+](=O)[O-])ccc3c2CCC3OP(=O)(N2CC2)N2CC2)c1] | LCMS (ESI): m/z 473.25 [M + H]⁺; ¹H NMR (400 MHz, DMSO-d₆) δ 8.04 (d, J = 8.2 Hz, 1H), 7.55 (d, J = 8.2 Hz, 1H), 7.43 (t, J = 7.9 Hz, 1H), 7.13 (dt, J = 7.6, 1.3 Hz, 1H), 7.01 (dd, J = 7.9, 2.7 Hz, 1H), 6.91-6.85 (m, 1H), 5.94 (q, J = 6.5 Hz, 1H), 2.94 (s, 3H), 2.85 (s, 3H), 2.78-2.66 (m, 1H), 2.62-2.52 (m, 2H), 2.26-1.98 (m, 9H). ³¹P NMR (162 MHz, DMSO-d₆) δ 30.10 (s). ee: >99% |
| 34 | 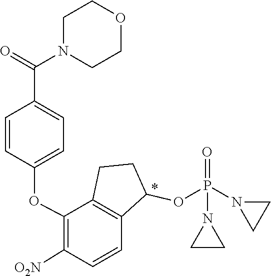 [SMILES: O=C(c1ccc(Oc2c([N+](=O)[O-])ccc3c2CCC3OP(=O)(N2CC2)N2CC2)cc1)N1CCOCC1] | LCMS (ESI): m/z 515.00 [M + H]⁺; ¹H NMR (400 MHz, DMSO-d₆) δ 8.06 (d, J = 8.4 Hz, 1H), 7.57 (d, J = 8.4 Hz, 1H), 7.47-7.39 (m, 2H), 7.00-6.92 (m, 2H), 5.95 (q, J = 6.8 Hz, 1H), 3.72-3.41 (m, 8H), 2.79-2.66 (m, 1H), 2.63-2.53 (m, 2H), 2.20- 2.03 (m, 9H). ³¹P NMR (162 MHz, DMSO-d₆) δ 30.18 (s). ee: >99% Retention time: 0.887 min. |
| 35 | 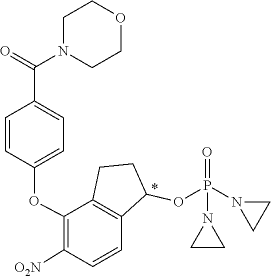 [SMILES: O=C(c1ccc(Oc2c([N+](=O)[O-])ccc3c2CCC3OP(=O)(N2CC2)N2CC2)cc1)N1CCOCC1] | LCMS (ESI): m/z 515.00 [M + H]⁺; ¹H NMR (400 MHz, DMSO-d₆) δ 8.06 (d, J = 8.4 Hz, 1H), 7.57 (d, J = 8.4 Hz, 1H), 7.45-7.40 (m, 2H), 6.98-6.93 (m, 2H), 5.95 (q, J = 6.8 Hz, 1H), 3.78-3.37 (m, 8H), 2.79-2.66 (m, 1H), 2.63-2.52 (m, 2H), 2.21- 2.03 (m, 9H). ³¹P NMR (162 MHz, DMSO-d₆) δ 30.17 (s). ee: >99% Retention time: 1.068 min. |
| 36 | 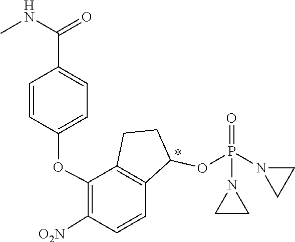 [SMILES: CNC(=O)c1ccc(Oc2c([N+](=O)[O-])ccc3c2CCC3OP(=O)(N2CC2)N2CC2)cc1] | LCMS (ESI): m/z 459.00 [M + H]⁺; ¹H NMR (400 MHz, DMSO-d₆) δ 8.38 (q, J = 4.0 Hz, 1H), 8.05 (d, J = 8.0 Hz, 1H), 7.87-7.79 (m, 2H), 7.57 (d, J = 8.0 Hz, 1H), 7.01-6.90 (m, 2H), 5.95 (q, J = 6.8 Hz, 1H), 2.77 (d, J = 4.5 Hz, 3H), 2.73-2.65 (m, 1H), 2.61-2.51 (m, 2H), 2.20-2.02 (m, 9H). ³¹P NMR (162 MHz, DMSO-d₆) δ 30.15 (s). ee: >99% Retention time: 5.260 min. |
| 37 | 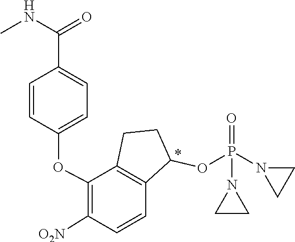 [SMILES: CNC(=O)c1ccc(Oc2c([N+](=O)[O-])ccc3c2CCC3OP(=O)(N2CC2)N2CC2)cc1] | LCMS (ESI): m/z 459.00 [M + H]⁺; ¹H NMR (400 MHz, DMSO-d₆) δ 8.38 (q, J = 4.0 Hz, 1H), 8.06 (d, J = 8.0 Hz, 1H), 7.86-7.78 (m, 2H), 7.57 (d, J = 8.0 Hz, 1H), 7.01-6.91 (m, 2H), 5.95 (q, J = 6.8 Hz, 1H), 2.79-2.66 (m, 4H), 2.60-2.52 (m, 2H), 2.20-2.02 (m, 9H). ³¹P NMR (162 MHz, DMSO-d₆) δ 30.15 (s). ee: >99% Retention time: 6.651 min. |
| 38 | 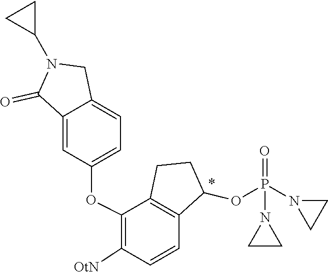 | LCMS (ESI): m/z 497.25 [M + H]⁺; ¹H NMR (400 MHz, DMSO-d₆) δ 8.06 (d, J = 8.2 Hz, 1H), 7.68-7.48 (m, 2H), 7.26 (dd, J = 8.3, 2.5 Hz, 1H), 6.95 (d, J = 2.5 Hz, 1H), 5.96 (q, J = 6.5, 6.0 Hz, 1H), 4.37 (s, 2H), 3.02-2.84 (m, 1H), 2.83-2.62 (m, 1H), 2.61-2.51 (m, 2H), 2.29-1.88 (m, 9H), 0.93-0.68 (m, 4H). ³¹P NMR (162 MHz, DMSO-d₆) δ 30.09 (s). ee: >99% |
| 39 | 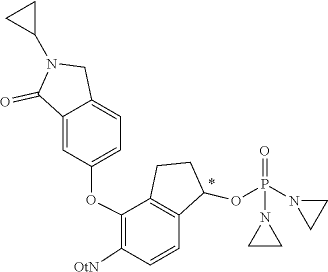 | LCMS (ESI): m/z 497.25 [M + H]⁺; ¹H NMR (400 MHz, DMSO-d₆) δ 8.06 (d, J = 8.2 Hz, 1H), 7.64-7.52 (m, 2H), 7.27 (dd, J = 8.3, 2.5 Hz, 1H), 6.95 (d, J = 2.4 Hz, 1H), 6.03- 5.90 (m, 1H), 4.37 (s, 2H), 3.00-2.85 (m, 1H), 2.81-2.65 (m, 1H), 2.61- 2.52 (m, 2H), 2.23-1.97 (m, 9H), 0.93-0.71 (m, 4H). ³¹P NMR (162 MHz, DMSO-d₆) δ 30.09 (s). ee: >99% |
| 40 | 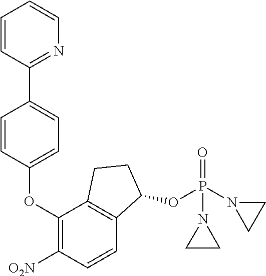 [SMILES: O=[N+]([O-])c1ccc2c(c1Oc1ccc(-c3ccccn3)cc1)CC[C@@H]2OP(=O)(N1CC1)N1CC1] | LCMS (ESI): m/z 479.1 [M + H]⁺; ¹H NMR (400 MHz, CDCl₃) δ 8.58 (d, J = 4.6 Hz, 1H), 7.92-7.79 (m, 3H), 7.71-7.56 (m, 2H), 7.45 (d, J = 8.2 Hz, 1H), 7.16-7.10 (m, 1H), 6.93-6.85 (m, 2H), 6.00-5.85 (m, 1H), 2.91-2.69 (m, 1H), 2.61-2.41 (m, 2H), 2.21-2.10 (m, 9H). ³¹P NMR (162 MHz, CDCl₃) δ 29.90 (s). ee: >99% Retention time: 1.941 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 41 | 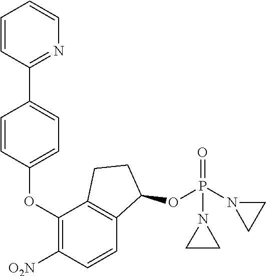 [SMILES: O=[N+]([O-])c1ccc2c(c1Oc1ccc(-c3ccccn3)cc1)CC[C@H]2OP(=O)(N1CC1)N1CC1] | LCMS (ESI): m/z 479.1 [M + H]⁺; ¹H NMR (400 MHz, CDCl₃) δ 8.67 (d, J = 4.6 Hz, 1H), 7.99-7.86 (m, 3H), 7.78-7.71 (m, 1H), 7.71-7.64 (m, 1H), 7.54 (d, J = 8.2 Hz, 1H), 7.26-7.19 (m, 1H), 7.05-6.91 (m, 2H), 6.10-5.89 (m, 1H), 2.95-2.83 (m, 1H), 2.70-2.49 (m, 2H), 2.32-2.18 (m, 9H). ³¹P NMR (162 MHz, CDCl₃) δ 29.91 (s). ee: 95% Retention time: 2.317 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 42 | 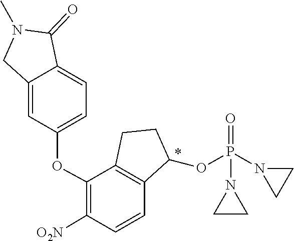 [SMILES: CN1Cc2cc(Oc3c([N+](=O)[O-])ccc4c3CCC4OP(=O)(N3CC3)N3CC3)ccc2C1=O] | LCMS (ESI): m/z 471.15 [M + H]⁺; ¹H NMR (400 MHz, DMSO-d₆) δ 8.08 (d, J = 8.2 Hz, 1H), 7.64 (d, J = 8.3 Hz, 1H), 7.58 (dd, J = 8.2, 0.8 Hz, 1H), 7.12 (d, J = 2.2 Hz, 1H), 7.02 (dd, J = 8.3, 2.4 Hz, 1H), 5.95 (q, J = 6.6 Hz, 1H), 4.40 (s, 2H), 3.04 (s, 3H), 2.80-2.44 (m, 3H), 2.22-1.98 (m, 9H). ee: >99% |
| 43 | 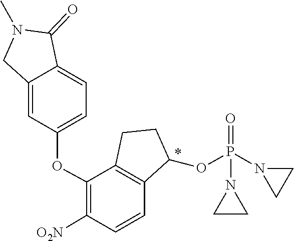 [SMILES: CN1Cc2cc(Oc3c([N+](=O)[O-])ccc4c3CCC4OP(=O)(N3CC3)N3CC3)ccc2C1=O] | LCMS (ESI): m/z 471.15 [M + H]⁺; ¹H NMR (400 MHz, DMSO-d₆) δ 8.08 (d, J = 8.2 Hz, 1H), 7.64 (d, J = 8.3 Hz, 1H), 7.58 (d, J = 8.2 Hz, 1H), 7.12 (d, J = 2.3 Hz, 1H), 7.02 (dd, J = 8.3, 2.3 Hz, 1H), 5.95 (q, J = 6.5 Hz, 1H), 4.40 (s, 2H), 3.04 (s, 3H), 2.79-2.43 (m, 3H), 2.28-1.98 (m, 9H).. ee: >99% |
| 44 | 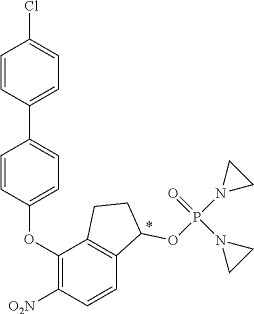 [SMILES: O=[N+]([O-])c1ccc2c(c1Oc1ccc(-c3ccc(Cl)cc3)cc1)CCC2OP(=O)(N1CC1)N1CC1] | LCMS (ESI): m/z 512.1 [M + H]⁺; ¹H NMR (400 MHz, CDCl₃) δ 7.93 (d, J = 8.2 Hz, 1H), 7.55 (d, J = 8.1 Hz, 1H), 7.61-7.48 (m, 4H), 7.41 (d, J = 8.5 Hz, 2H), 6.95 (d, J = 8.7 Hz, 2H), 6.06-6.01 (m, 1H), 3.00-2.88 (m, 1H), 2.75-2.54 (m, 2H), 2.38-2.17 (m, 9H). ³¹P NMR (162 MHz, CDCl₃) δ 30.04 (s). ee: >99% Retention time: 4.962 min. |
| 45 | 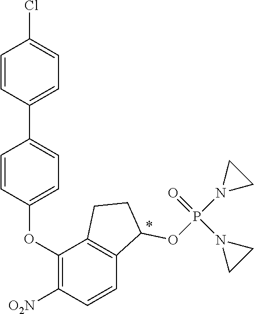 [SMILES: O=[N+]([O-])c1ccc2c(c1Oc1ccc(-c3ccc(Cl)cc3)cc1)CCC2OP(=O)(N1CC1)N1CC1] | LCMS (ESI): m/z 512 [M + H]⁺; ¹H NMR (400 MHz, CDCl₃) δ 7.93 (d, J = 8.2 Hz, 1H), 7.55 (d, J = 8.1 Hz, 1H), 7.61-7.48 (m, 4H), 7.41 (d, J = 8.5 Hz, 2H), 6.95 (d, J = 8.7 Hz, 2H), 6.06-6.01 (m, 1H), 3.00-2.88 (m, 1H), 2.75-2.54 (m, 2H), 2.38-2.17 (m, 9H). ³¹P NMR (162 MHz, CDCl₃) δ 30.04 (s). ee: >99% Retention time: 8.240 min. |
| 46 | 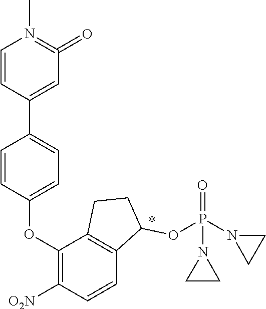 | LCMS (ESI): m/z 512 [M + H]⁺; ¹H NMR (400 MHz, CDCl₃) δ 7.93 (d, J = 8.2 Hz, 1H), 7.55 (d, J = 8.1 Hz, 1H), 7.61-7.48 (m, 4H), 7.41 (d, J = 8.5 Hz, 2H), 6.95 (d, J = 8.7 Hz, 2H), 6.06-6.01 (m, 1H), 3.00-2.88 (m, 1H), 2.75-2.54 (m, 2H), 2.38-2.17 (m, 9H). ³¹P NMR (162 MHz, CDCl₃) δ 30.05 (s). ee: >99% Retention time: 1.897 min. |
| 47 | 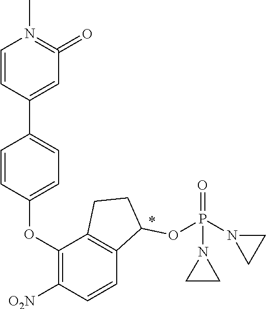 | LCMS (ESI): m/z 509.1 [M + H]⁺; ¹H NMR (400 MHz, CDCl₃) δ 7.92 (d, J = 8.2 Hz, 1H), 7.59-7.48 (m, 3H), 7.33 (d, J = 7.1 Hz, 1H), 6.94 (d, J = 8.8 Hz, 2H), 6.75 (d, J = 1.8 Hz, 1H), 6.40 (dd, J = 7.1, 2.0 Hz, 1H), 6.08-5.95 (m, 1H), 3.58 (s, 1H), 2.96-2.83 (m, 1H), 2.71-2.53 (m, 2H), 2.32-2.13 (m, 9H). ³¹P NMR (162 MHz, CDCl₃) δ 30.04 (s). ee: 99% Retention time: 2.848 min. |
| 48 | 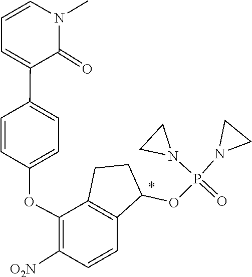 [SMILES: Cn1cccc(-c2ccc(Oc3c([N+](=O)[O-])ccc4c3CCC4OP(=O)(N3CC3)N3CC3)cc2)c1=O] | LCMS (ESI): m/z 509.2 [M + H]⁺; ¹H NMR (400 MHz, CDCl₃) δ 7.92-7.85 (m, 1H), 7.69-7.63 (m, 2H), 7.54-7.49 (m, 1H), 7.47 (dd, J = 7.0, 2.0 Hz, 1H), 7.30 (dd, J = 6.7, 2.0 Hz, 1H), 6.93-6.86 (m, 2H), 6.25 (t, J = 6.8 Hz, 1H), 6.03-5.95 (m, 1H), 3.61 (s, 3H), 2.93-2.84 (m, 1H), 2.68-2.49 (m, 2H), 2.29-2.15 (m, 9H). ³¹P NMR (162 MHz, CDCl₃) δ 29.85 (s). ee: 95% Retention time: 3.509 min. |
| 49 | 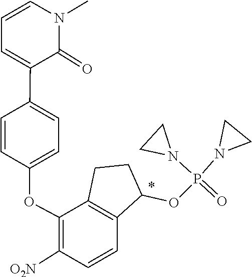 [SMILES: Cn1cccc(-c2ccc(Oc3c([N+](=O)[O-])ccc4c3CCC4OP(=O)(N3CC3)N3CC3)cc2)c1=O] | LCMS (ESI): m/z 509.1 [M + H]⁺; ¹H NMR (400 MHz, CDCl₃) δ 7.89 (d, J = 8.2 Hz, 1H), 7.69-7.63 (m, 2H), 7.53-7.43 (m, 2H), 7.30 (dd, J = 6.7, 2.0 Hz, 1H), 6.91-6.86 (m, 2H), 6.25 (t, J = 6.8 Hz, 1H), 6.03-5.96 (m, 1H), 3.61 (s, 3H), 2.93-2.84 (m, 1H), 2.68-2.49 (m, 2H), 2.30-2.15 (m, 9H). ³¹P NMR (162 MHz, CDCl₃) δ 29.84 (s). ee: >99% Retention time: 4.359 min. |
| 50 | 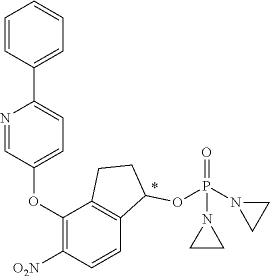 [SMILES: O=[N+]([O-])c1ccc2c(c1Oc1ccc(-c3ccccc3)nc1)CCC2OP(=O)(N1CC1)N1CC1] | LCMS (ESI): m/z 479.1 [M + H]⁺; ¹H NMR (400 MHz, CDCl₃) δ 8.36 (d, J = 2.9 Hz, 1H), 7.97-7.90 (m, 3H), 7.69 (d, J = 8.7 Hz, 1H), 7.58 (d, J = 8.2 Hz, 1H), 7.50-7.43 (m, 2H), 7.43-7.37 (m, 1H), 7.26-7.23 (m, 1H), 6.06-5.98 (m, 1H), 2.98- 2.87 (m, 1H), 2.74-2.53 (m, 2H), 2.32-2.16 (m, 9H) ³¹P NMR (162 MHz, CDCl₃) δ 30.00 (s). ee: >99% Retention time: 6.294 min. |
| 51 | 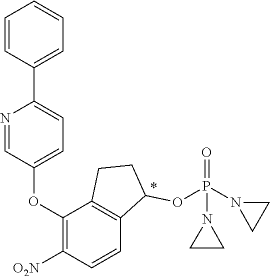 [SMILES: O=[N+]([O-])c1ccc2c(c1Oc1ccc(-c3ccccc3)nc1)CCC2OP(=O)(N1CC1)N1CC1] | LCMS (ESI): m/z 479.1 [M + H]⁺; ¹H NMR (400 MHz, CDCl₃) δ 8.36 (d, J = 2.8 Hz, 1H), 7.97-7.90 (m, 3H), 7.69 (d, J = 8.6 Hz, 1H), 7.58 (d, J = 8.2 Hz, 1H), 7.49-7.43 (m, 2H), 7.42-7.37 (m, 1H), 7.24 (d, J = 3.0 Hz, 1H), 6.06-5.99 (m, 1H), 2.98-2.87 (m, 1H), 2.72-2.53 (m, 2H), 2.31-2.16 (m, 9H). ³¹P NMR (162 MHz, CDCl₃) δ 30.00 (s). ee: >99% Retention time: 10.785 min. |
| 52 | 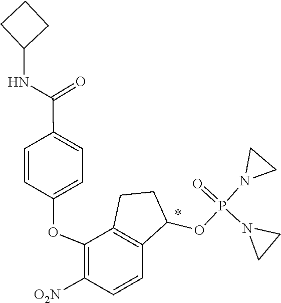 [SMILES: O=C(NC1CCC1)c1ccc(Oc2c([N+](=O)[O-])ccc3c2CCC3OP(=O)(N2CC2)N2CC2)cc1] | LCMS (ESI): m/z 499.2 [M + H]⁺; ¹H NMR (400 MHz, CDCl₃) δ 7.92 (d, J = 8.2 Hz, 1H), 7.80-7.65 (m, 2H), 7.55 (d, J = 8.2 Hz, 1H), 6.93-6.86 (m, 2H), 6.17 (d, J = 7.4 Hz, 1H), 6.08-5.95 (m, 1H), 4.65-4.50 (m, 1H), 2.90-2.79 (m, 1H), 2.69- 2.50 (m, 2H), 2.47-2.37 (m, 2H), 2.33-2.11 (m, 9H), 2.02-1.85 (m, 2H), 1.86-1.70 (m, 2H). ³¹P NMR (162 MHz, CDCl₃) δ 30.01 (s). ee: >99% Retention time: 5.903 min. |
| 53 | 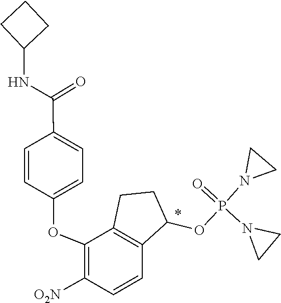 [SMILES: O=C(NC1CCC1)c1ccc(Oc2c([N+](=O)[O-])ccc3c2CCC3OP(=O)(N2CC2)N2CC2)cc1] | LCMS (ESI): m/z 499.2 [M + H]⁺; ¹H NMR (400 MHz, CDCl₃) δ 7.92 (d, J = 8.2 Hz, 1H), 7.72 (d, J = 8.7 Hz, 2H), 7.55 (d, J = 8.2 Hz, 1H), 6.96-6.84 (m, 2H), 6.17 (d, J = 7.4 Hz, 1H), 6.06-5.94 (m, 1H), 4.63-4.51 (m, 1H), 2.95-2.78 (m, 1H), 2.69-2.49 (m, 2H), 2.48-2.36 (m, 2H), 2.33-2.09 (m, 9H), 2.06-1.87 (m, 2H), 1.87-1.73 (m, 2H). ³¹P NMR (162 MHz, CDCl₃) δ 30.00 (s). ee: >99% Retention time: 8.394 min. |
| 54 | 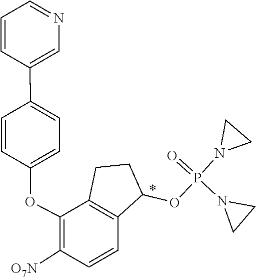 | LCMS (ESI): m/z 479.2 [M + H]⁺; ¹H NMR (400 MHz, CDCl₃) δ 8.83-8.77 (m, 1H), 8.57 (dd, J = 4.8, 1.5 Hz, 1H), 7.92 (d, J = 8.2 Hz, 1H), 7.87-7.79 (m, 1H), 7.57-7.49 (m, 3H), 7.37-7.32 (m, 1H), 6.98 (d, J = 8.8 Hz, 2H), 6.07-5.98 (m, 1H), 2.99-2.86 (m, 1H), 2.72-2.52 (m, 2H), 2.32-2.14 (m, 9H). ³¹P NMR (162 MHz, CDCl₃) δ 30.04 (s). ee: >99% Retention time: 4.620 min. |
| 55 | 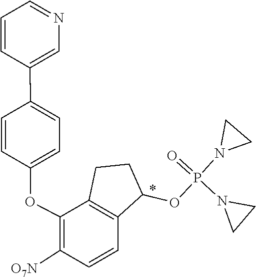 | LCMS (ESI): m/z 499.2 [M + H]⁺; ¹H NMR (400 MHz, CDCl₃) δ 8.81 (d, J = 1.9 Hz, 1H), 8.58 (dd, J = 4.8, 1.5 Hz, 1H), 7.92 (d, J = 8.2 Hz, 1H), 7.88-7.79 (m, 1H), 7.57-7.48 (m, 3H), 7.39-7.31 (m, 1H), 7.02-6.92 (m, 2H), 6.07-5.98 (m, 1H), 2.99-2.87 (m, 1H), 2.73-2.52 (m, 2H), 2.33-2.15 (m, 9H). ³¹P NMR (162 MHz, CDCl₃) δ 30.05 (s). ee: >99% Retention time: 4.860 min. |
| 56 | 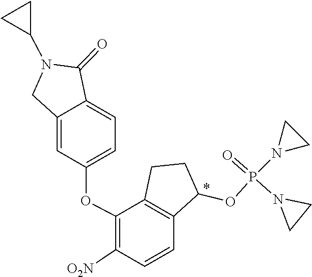 [SMILES: O=C1c2ccc(Oc3c([N+](=O)[O-])ccc4c3CCC4OP(=O)(N3CC3)N3CC3)cc2CN1C1CC1] | LCMS (ESI): m/z 497.2 [M + H]⁺; ¹H NMR (400 MHz, CDCl₃) δ 7.93 (d, J = 8.2 Hz, 1H), 7.76 (d, J = 8.3 Hz, 1H), 7.56 (d, J = 8.2 Hz, 1H), 6.93 (dd, J = 8.4, 2.2 Hz, 1H), 6.89 (d, J = 1.7 Hz, 1H), 6.11-5.96 (m, 1H), 4.27 (s, 2H), 2.99-2.79 (m, 2H), 2.72-2.52 (m, 2H), 2.33-2.13 (m, 9H), 0.97-0.75 (m, 4H). ³¹P NMR (162 MHz, CDCl₃) δ 30.11 (s). ee: >99% Retention time: 7.211 min. |
| 57 | 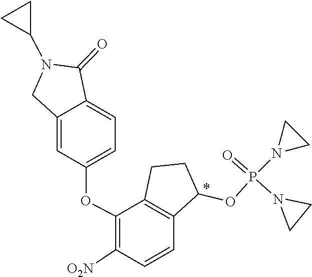 [SMILES: O=C1c2ccc(Oc3c([N+](=O)[O-])ccc4c3CCC4OP(=O)(N3CC3)N3CC3)cc2CN1C1CC1] | LCMS (ESI): m/z 497.2 [M + H]⁺; ¹H NMR (400 MHz, CDCl₃) δ 7.93 (d, J = 8.2 Hz, 1H), 7.76 (d, J = 8.3 Hz, 1H), 7.56 (d, J = 8.2 Hz, 1H), 6.93 (dd, J = 8.3, 2.2 Hz, 1H), 6.89 (d, J = 1.8 Hz, 1H), 6.07-5.99 (m, 1H), 4.27 (s, 2H), 2.98-2.74 (m, 2H), 2.70-2.50 (m, 2H), 2.34-2.14 (m, 9H), 0.96-0.82 (m, 4H). ³¹P NMR (162 MHz, CDCl₃) δ 30.12 (s). ee: >99% Retention time: 11.589 min. |
| 58 | 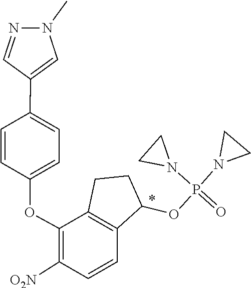 [SMILES: Cn1cc(-c2ccc(Oc3c([N+](=O)[O-])ccc4c3CCC4OP(=O)(N3CC3)N3CC3)cc2)cn1] | LCMS (ESI): m/z 482.2 [M + H]⁺; ¹H NMR (400 MHz, CDCl₃) δ 7.88 (d, J = 8.2 Hz, 1H), 7.69 (s, 1H), 7.55 (s, 1H), 7.50 (d, J = 8.2 Hz, 1H), 7.41-7.35 (m, 2H), 6.90-6.84 (m, 2H), 6.05-5.95 (m, 1H), 3.93 (s, 3H), 2.93-2.82 (m, 1H), 2.66-2.50 (m, 2H), 2.29-2.18 (m, 9H). ³¹P NMR (162 MHz, CDCl₃) δ 29.97 (s). ee: >99% Retention time: 2.395 min. |
| 59 | 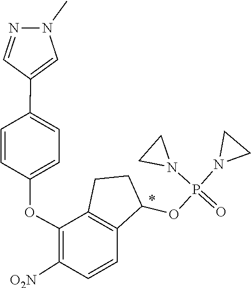 [SMILES: Cn1cc(-c2ccc(Oc3c([N+](=O)[O-])ccc4c3CCC4OP(=O)(N3CC3)N3CC3)cc2)cn1] | LCMS (ESI): m/z 482.2 [M + H]⁺; ¹H NMR (400 MHz, CDCl₃) δ 7.88 (d, J = 8.2 Hz, 1H), 7.69 (s, 1H), 7.55 (s, 1H), 7.50 (d, J = 8.2 Hz, 1H), 7.41-7.36 (m, 2H), 6.90-6.83 (m, 2H), 6.05-5.96 (m, 1H), 3.93 (s, 3H), 2.92-2.82 (m, 1H), 2.65-2.51 (m, 2H), 2.32-2.17 (m, 9H). ³¹P NMR (162 MHz, CDCl₃) δ 29.99 (s). ee: >99% Retention time: 3.070 min. |
| 60 | 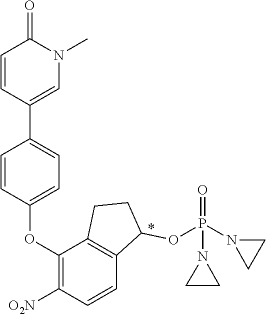 [SMILES: Cn1cc(-c2ccc(Oc3c([N+](=O)[O-])ccc4c3CCC4OP(=O)(N3CC3)N3CC3)cc2)ccc1=O] | LCMS (ESI): m/z 509.2 [M + H]⁺; ¹H NMR (400 MHz, CDCl₃) δ 7.87 (d, J = 8.2 Hz, 1H), 7.58-7.48 (m, 2H), 7.43 (d, J = 2.3 Hz, 1H), 7.29 (d, J = 8.7 Hz, 2H), 6.88 (d, J = 8.6 Hz, 2H), 6.62 (d, J = 9.4 Hz, 1H), 6.04-5.93 (m, 1H), 3.58 (s, 3H), 2.92-2.81 (m, 1H), 2.66-2.50 (m, 2H), 2.25-2.17 (m, 8H).. ³¹P NMR (162 MHz, CDCl₃) δ 30.06 (s). ee: >99% Retention time: 4.655 min. |
| 61 | 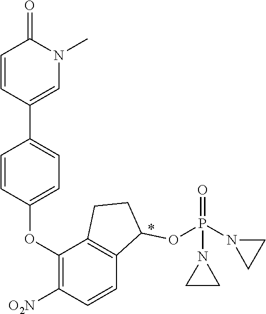 [SMILES: Cn1cc(-c2ccc(Oc3c([N+](=O)[O-])ccc4c3CCC4OP(=O)(N3CC3)N3CC3)cc2)ccc1=O] | LCMS (ESI): m/z 509.2 [M + H]⁺; ¹H NMR (400 MHz, CDCl₃) δ 7.82 (d, J = 8.2 Hz, 1H), 7.52-7.44 (m, 2H), 7.39 (d, J = 2.5 Hz, 1H), 7.25 (d, J = 8.7 Hz, 2H), 6.83 (d, J = 8.7 Hz, 2H), 6.57 (d, J = 9.4 Hz, 1H), 5.97-5.91 (m, 1H), 3.54 (s, 3H), 2.86-2.78 (m, 1H), 2.60-2.46 (m, 2H), 2.21-2.12 (m, 9H). ³¹P NMR (162 MHz, CDCl₃) δ 30.05 (s). ee: >99% Retention time: 6.684 min. |
| 62 | 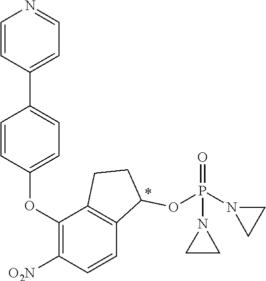 [SMILES: O=[N+]([O-])c1ccc2c(c1Oc1ccc(-c3ccncc3)cc1)CCC2OP(=O)(N1CC1)N1CC1] | LCMS (ESI): m/z 479.2 [M + H]⁺; ¹H NMR (400 MHz, CDCl₃) δ 8.65-8.61 (m, 2H), 7.93 (d, J = 8.2 Hz, 1H), 7.62-7.58 (m, 2H), 7.55 (d, J = 8.3 Hz, 1H), 7.49-7.45 (m, 2H), 6.98 (d, J = 8.8 Hz, 2H), 6.07-6.00 (m, 1H), 2.97-2.87 (m, 1H), 2.70- 2.56 (m, 2H), 2.28-2.20 (m, 9H). ³¹P NMR (162 MHz, CDCl₃) δ 30.07 (s). ee: >99% Retention time: 3.597 min. |
| 63 | 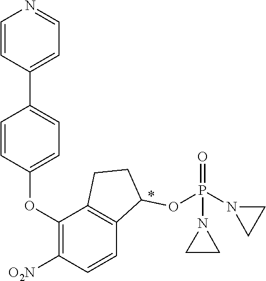 [SMILES: O=[N+]([O-])c1ccc2c(c1Oc1ccc(-c3ccncc3)cc1)CCC2OP(=O)(N1CC1)N1CC1] | LCMS (ESI): m/z 479.2 [M + H]⁺; ¹H NMR (400 MHz, CDCl₃) δ 8.65-8.59 (m, 2H), 7.93 (d, J = 8.2 Hz, 1H), 7.62-7.57 (m, 2H), 7.55 (d, J = 8.2 Hz, 1H), 7.48-7.44 (m, 2H), 7.01-6.94 (m, 2H), 6.06-5.99 (m, 1H), 2.97-2.87 (m, 1H), 2.71-2.56 (m, 2H), 2.31-2.14 (m, 9H). ³¹P NMR (162 MHz, CDCl₃) δ 30.07 (s). ee: >88% Retention time: 4.683 min. |
| 64 | 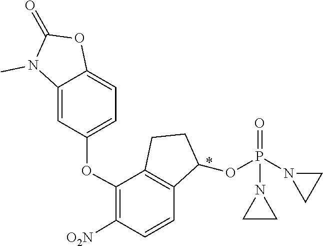 [SMILES: Cn1c(=O)oc2ccc(Oc3c([N+](=O)[O-])ccc4c3CCC4OP(=O)(N3CC3)N3CC3)cc21] | LCMS (ESI): m/z 473.1 [M + H]⁺; ¹H NMR (400 MHz, CDCl₃) δ 7.90 (d, J = 8.2 Hz, 1H), 7.54 (d, J = 8.3 Hz, 1H), 7.08 (d, J = 8.7 Hz, 1H), 6.62 (t, J = 3.4 Hz, 1H), 6.52 (dd, J = 8.7, 2.5 Hz, 1H), 6.05-5.97 (m, 1H), 3.37 (s, 3H), 2.93-2.82 (m, 1H), 2.66-2.50 (m, 2H), 2.30-2.14 (m, 9H). ³¹P NMR (162 MHz, CDCl₃) δ 30.13 (s). ee: >99% Retention time: 4.259 min. |
| 65 | 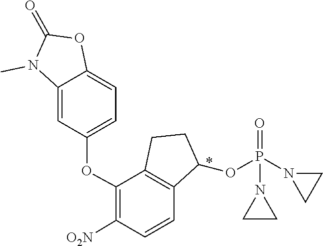 [SMILES: Cn1c(=O)oc2ccc(Oc3c([N+](=O)[O-])ccc4c3CCC4OP(=O)(N3CC3)N3CC3)cc21] | LCMS (ESI): m/z 473.1 [M + H]⁺; ¹H NMR (400 MHz, CDCl₃) δ 7.93-7.87 (m, 1H), 7.56-7.49 (m, 1H), 7.11-7.04 (m, 1H), 6.66-6.59 (m, 1H), 6.55-6.48 (m, 1H), 6.04-5.98 (m, 1H), 3.40-3.32 (m, 3H), 2.93-2.82 (m, 1H), 2.66-2.51 (m, 2H), 2.33-2.14 (m, 9H). ³¹P NMR (162 MHz, CDCl₃) δ 30.13 (s). ee: >99% Retention time: 5.464 min. |
| 66 | 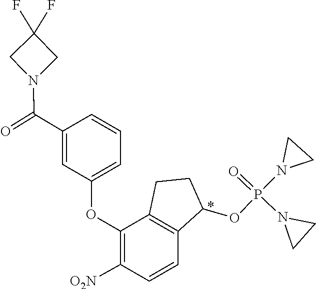 [SMILES: O=C(c1cccc(Oc2c([N+](=O)[O-])ccc3c2CCC3OP(=O)(N2CC2)N2CC2)c1)N1CC(F)(F)C1] | LCMS (ESI): m/z 521.1 [M + H]⁺; ¹H NMR (400 MHz, CDCl₃) δ 7.92 (d, J = 8.2 Hz, 1H), 7.56 (d, J = 8.2 Hz, 1H), 7.43-7.36 (m, 1H), 7.33-7.28 (m, 1H), 7.13-7.10 (m, 1H), 7.07-7.01 (m, 1H), 6.06-5.97 (m, 1H), 4.50 (t, J = 12.0 Hz, 4H), 2.92- 2.81 (m, 1H), 2.67-2.52 (m, 2H), 2.28-2.15 (m, 9H). ³¹P NMR (162 MHz, CDCl₃) δ 29.97 (s). ee: >99% Retention time: 2.789 min. |
| 67 | 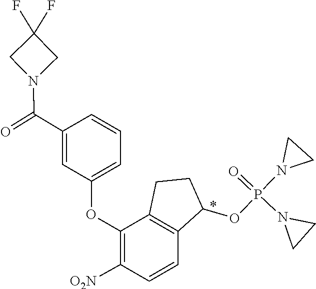 [SMILES: O=C(c1cccc(Oc2c([N+](=O)[O-])ccc3c2CCC3OP(=O)(N2CC2)N2CC2)c1)N1CC(F)(F)C1] | LCMS (ESI): m/z 521.1 [M + H]⁺; ¹H NMR (400 MHz, CDCl₃) δ 7.92 (d, J = 8.2 Hz, 1H), 7.56 (d, J = 8.2 Hz, 1H), 7.39 (t, J = 7.9 Hz, 1H), 7.33-7.29 (m, 1H), 7.13-7.10 (m, 1H), 7.06-7.02 (m, 1H), 6.08-5.96 (m, 1H), 4.50 (t, J = 12.0 Hz, 4H), 2.92-2.82 (m, 1H), 2.67-2.53 (m, 2H), 2.31-2.15 (m, 9H). ³¹P NMR (162 MHz, CDCl₃) δ 29.96 (s). ee: >99% Retention time: 3.530 min. |
| 68 | 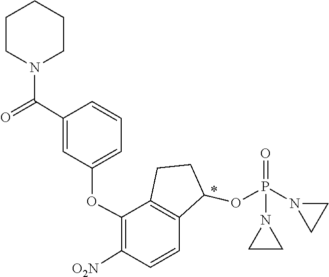 [SMILES: O=C(c1cccc(Oc2c([N+](=O)[O-])ccc3c2CCC3OP(=O)(N2CC2)N2CC2)c1)N1CCCCC1] | LCMS (ESI): m/z 513.4 [M + H]⁺; ¹H NMR (400 MHz, CDCl₃) δ 7.87 (d, J = 8.2 Hz, 1H), 7.51 (d, J = 8.2 Hz, 1H), 7.32 (t, J = 7.9 Hz, 1H), 7.10-7.05 (m, 1H), 6.95-6.88 (m, 1H), 6.84-6.80 (m, 1H), 6.02-5.95 (m, 1H), 3.72-3.57 (m, 2H), 3.35-3.20 (m, 2H), 2.91-2.79 (m, 1H), 2.65-2.48 (m, 2H), 2.26-2.16 (m, 9H), 1.70- 1.56 (m, 4H), 1.53-1.41 (m, 2H). ³¹P NMR (162 MHz, CDCl₃) δ 29.86 (s). ee: >99% Retention time: 2.513 min. |
| 69 | 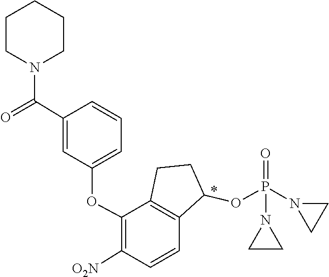 [SMILES: O=C(c1cccc(Oc2c([N+](=O)[O-])ccc3c2CCC3OP(=O)(N2CC2)N2CC2)c1)N1CCCCC1] | LCMS (ESI): m/z 513.4 [M + H]⁺; ¹H NMR (400 MHz, CDCl₃) δ 7.89 (d, J = 8.2 Hz, 1H), 7.53 (d, J = 8.2 Hz, 1H), 7.34 (t, J = 7.9 Hz, 1H), 7.12-7.07 (m, 1H), 6.96-6.91 (m, 1H), 6.86-6.82 (m, 1H), 6.04-5.97 (m, 1H), 3.76-3.57 (m, 2H), 3.41-3.24 (m, 2H), 2.94-2.81 (m, 1H), 2.69-2.50 (m, 2H), 2.32-2.13 (m, 9H), 1.71-1.58 (m, 4H), 1.55-1.44 (m, 2H). ³¹P NMR (162 MHz, CDCl₃) δ 29.87 (s). ee: >99% Retention time: 2.746 min. |
| 70 | 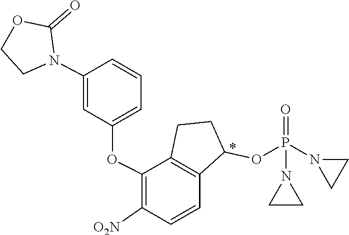 [SMILES: O=C1OCCN1c1cccc(Oc2c([N+](=O)[O-])ccc3c2CCC3OP(=O)(N2CC2)N2CC2)c1] | LCMS (ESI): m/z 487.1 [M + H]⁺; ¹H NMR (400 MHz, CDCl₃) δ 7.89 (d, J = 8.2 Hz, 1H), 7.52 (d, J = 8.2 Hz, 1H), 7.33-7.23 (m, 2H), 7.21-7.15 (m, 1H), 6.62-6.55 (m, 1H), 6.04-5.94 (m, 1H), 4.47 (t, J = 8.0 Hz, 2H), 4.09-3.97 (m, 2H), 2.97- 2.83 (m, 1H), 2.71-2.49 (m, 2H), 2.29-2.15 (m, 9H) ³¹P NMR (162 MHz, CDCl₃) δ 29.89 (s). ee: >99% Retention time: 5.822 min. |
| 71 | 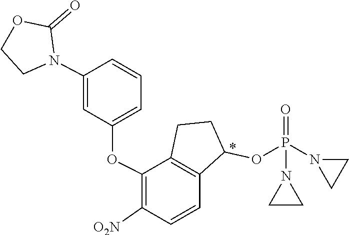 [SMILES: O=C1OCCN1c1cccc(Oc2c([N+](=O)[O-])ccc3c2CCC3OP(=O)(N2CC2)N2CC2)c1] | LCMS (ESI): m/z 487.1 [M + H]⁺; ¹H NMR (400 MHz, CDCl₃) δ 7.89 (d, J = 8.2 Hz, 1H), 7.56-7.49 (m, 1H), 7.34-7.21 (m, 2H), 7.21-7.12 (m, 1H), 6.62-6.53 (m, 1H), 6.05- 5.96 (m, 1H), 4.49-4.40 (m, 2H), 4.07-3.97 (m, 2H), 2.97-2.84 (m, 1H), 2.71-2.50 (m, 2H), 2.29-2.14 (m, 9H). ³¹P NMR (162 MHz, CDCl₃) δ 29.89 (s). ee: >99% Retention time: 7.707 min. |
| 72 | 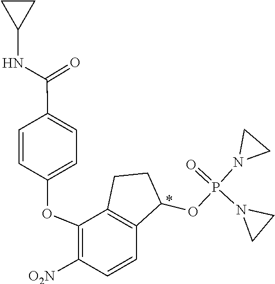 [SMILES: O=C(NC1CC1)c1ccc(Oc2c([N+](=O)[O-])ccc3c2CCC3OP(=O)(N2CC2)N2CC2)cc1] | LCMS (ESI): m/z 485.2 [M + H]⁺; ¹H NMR (400 MHz, CDCl₃) δ 7.91 (d, J = 8.2 Hz, 1H), 7.75-7.69 (m, 2H), 7.54 (d, J = 8.2 Hz, 1H), 6.89-6.84 (m, 2H), 6.45 (s, 1H), 6.03- 5.97 (m, 1H), 2.91-2.80 (m, 2H), 2.64-2.50 (m, 2H), 2.28-2.17 (m, 9H), 0.89-0.81 (m, 2H), 0.66-0.58 (m, 2H). ³¹P NMR (162 MHz, CDCl₃) δ 29.96 (s). ee: >99% Retention time: 4.522 min. |
| 73 | 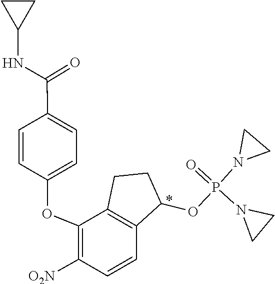 [SMILES: O=C(NC1CC1)c1ccc(Oc2c([N+](=O)[O-])ccc3c2CCC3OP(=O)(N2CC2)N2CC2)cc1] | LCMS (ESI): m/z 485.2 [M + H]⁺; ¹H NMR (400 MHz, CDCl₃) δ 7.91 (d, J = 8.2 Hz, 1H), 7.75-7.68 (m, 2H), 7.54 (d, 1H), 6.91-6.83 (m, 2H), 6.44 (s, 1H), 6.05-5.95 (m, 1H), 2.91-2.79 (m, 2H), 2.64-2.49 (m, 2H), 2.28-2.16 (m, 9H), 0.87-0.80 (m, 2H), 0.65-0.58 (m, 2H). ³¹P NMR (162 MHz, CDCl₃) δ 29.96 (s). ee: >99% Retention time: 4.970 min. |
| 74 | 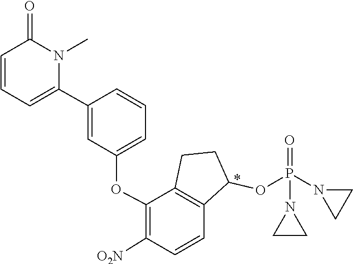 [SMILES: Cn1c(-c2cccc(Oc3c([N+](=O)[O-])ccc4c3CCC4OP(=O)(N3CC3)N3CC3)c2)cccc1=O] | LCMS (ESI): m/z 509.2 [M + H]⁺; ¹H NMR (400 MHz, CDCl₃) δ 7.91 (d, J = 8.2 Hz, 1H), 7.75-7.68 (m, 2H), 7.54 (d, 1H), 6.91-6.83 (m, 2H), 6.44 (s, 1H), 6.05-5.95 (m, 1H), 2.91-2.79 (m, 2H), 2.64-2.49 (m, 2H), 2.28-2.16 (m, 9H), 0.87-0.80 (m, 2H), 0.65-0.58 (m, 2H). ³¹P NMR (162 MHz, CDCl₃) δ 29.84 (s). ee: >99% Retention time: 2.436 min. |
| 75 | 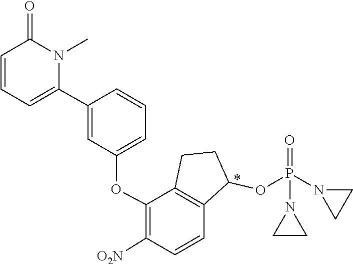 [SMILES: Cn1c(-c2cccc(Oc3c([N+](=O)[O-])ccc4c3CCC4OP(=O)(N3CC3)N3CC3)c2)cccc1=O] | LCMS (ESI): m/z 509.2 [M + H]⁺; ¹H NMR (400 MHz, CDCl₃) δ 7.86 (d, J = 8.2 Hz, 1H), 7.50-7.40 (m, 3H), 7.34-7.28 (m, 2H), 7.26-7.24 (m, 1H), 6.84-6.74 (m, 1H), 6.30- 6.20 (m, 1H), 6.02-5.96 (m, 1H), 3.60 (s, 3H), 2.97-2.82 (m, 1H), 2.70- 2.50 (m, 2H), 2.29-2.11 (m, 9H). ³¹P NMR (162 MHz, CDCl₃) δ 29.84 (s). ee: >99% Retention time: 2.855 min. |
| 76 | 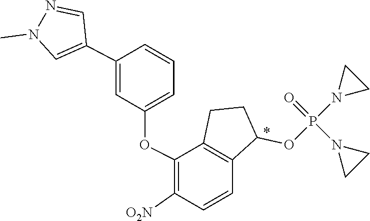 [SMILES: Cn1cc(-c2cccc(Oc3c([N+](=O)[O-])ccc4c3CCC4OP(=O)(N3CC3)N3CC3)c2)cn1] | LCMS (ESI): m/z 482.1 [M + H]⁺; ¹H NMR (400 MHz, CDCl₃) δ 7.89 (d, J = 8.2 Hz, 1H), 7.70 (s, 1H), 7.57 (s, 1H), 7.51 (d, J = 8.2 Hz, 1H), 7.26-7.24 (m, 1H), 7.20-7.16 (m, 1H), 7.08-6.95 (m, 1H), 6.71-6.60 (m, 1H), 6.04-5.95 (m, 1H), 3.93 (s, 3H), 2.93-2.84 (m, 1H), 2.68-2.47 (m, 2H), 2.28-2.13 (m, 9H). ³¹P NMR (162 MHz, CDCl₃) δ 29.98 (s). ee: >99% Retention time: 4.999 min. |
| 77 | 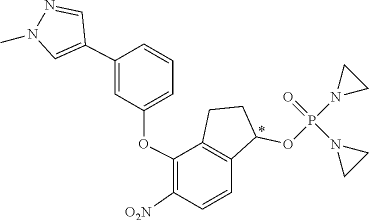 [SMILES: Cn1cc(-c2cccc(Oc3c([N+](=O)[O-])ccc4c3CCC4OP(=O)(N3CC3)N3CC3)c2)cn1] | LCMS (ESI): m/z 482.1 [M + H]⁺; ¹H NMR (400 MHz, CDCl₃) δ 7.89 (d, J = 8.2 Hz, 1H), 7.70 (s, 1H), 7.57 (s, 1H), 7.51 (d, J = 8.2 Hz, 1H), 7.29-7.26 (m, 1H), 7.19-7.16 (m, 1H), 7.04-7.01 (m, 1H), 6.68-6.62 (m, 1H), 6.04-5.96 (m, 1H), 3.93 (s, 3H), 2.94-2.84 (m, 1H), 2.67-2.50 (m, 2H), 2.31-2.15 (m, 9H). ³¹P NMR (162 MHz, CDCl₃) δ 29.98 (s). ee: >99% Retention time: 8.646 min. |
| 78 | 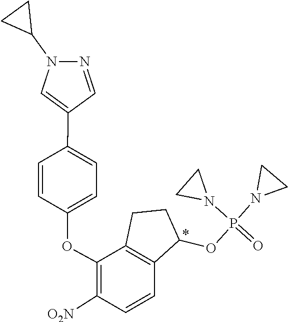 [SMILES: O=[N+]([O-])c1ccc2c(c1Oc1ccc(-c3cnn(C4CC4)c3)cc1)CCC2OP(=O)(N1CC1)N1CC1] | LCMS (ESI): m/z 508.2 [M + H]⁺; ¹H NMR (400 MHz, CDCl₃) δ 7.88 (d, J = 8.2 Hz, 1H), 7.68 (s, 1H), 7.63 (s, 1H), 7.50 (d, J = 8.2 Hz, 1H), 7.38 (d, J = 8.7 Hz, 2H), 6.87 (d, J = 8.8 Hz, 2H), 6.08-5.93 (m, 1H), 3.72-3.52 (m, 1H), 2.94-2.76 (m, 1H), 2.67-2.46 (m, 2H), 2.31-2.13 (m, 9H), 1.20-1.01 (m, 4H). ³¹P NMR (162 MHz, CDCl₃) δ 29.98 (s). ee: >99% Retention time: 4.236 min. |
| 79 | 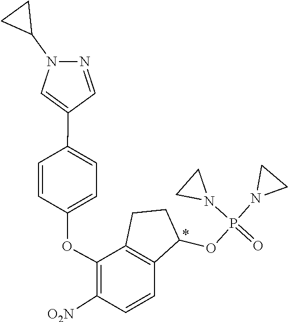 [SMILES: O=[N+]([O-])c1ccc2c(c1Oc1ccc(-c3cnn(C4CC4)c3)cc1)CCC2OP(=O)(N1CC1)N1CC1] | LCMS (ESI): m/z 508.2 [M + H]⁺; ¹H NMR (400 MHz, CDCl₃) δ 7.88 (d, J = 8.2 Hz, 1H), 7.68 (s, 1H), 7.63 (s, 1H), 7.50 (d, J = 8.2 Hz, 1H), 7.38 (d, J = 8.7 Hz, 2H), 6.87 (d, J = 8.7 Hz, 2H), 6.07-5.93 (m, 1H), 3.69-3.55 (m, 2H), 2.94-2.77 (m, 1H), 2.72-2.45 (m, 2H), 2.31-2.10 (m, 8H), 1.22-0.98 (m, 4H). ³¹P NMR (162 MHz, CDCl₃) δ 29.98 (s). ee: >99% Retention time: 5.714 min. |
| 80 | 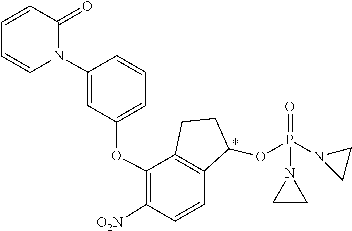 [SMILES: O=c1ccccn1-c1cccc(Oc2c([N+](=O)[O-])ccc3c2CCC3OP(=O)(N2CC2)N2CC2)c1] | LCMS (ESI): m/z 495.1 [M + H]⁺; ¹H NMR (400 MHz, CDCl₃) δ 7.88 (d, J = 8.2 Hz, 1H), 7.57-7.49 (m, 1H), 7.46-7.30 (m, 3H), 7.13-7.05 (m, 1H), 6.99-6.87 (m, 2H), 6.61 (d, J = 9.2 Hz, 1H), 6.28-6.19 (m, 1H), 6.05-5.97 (dd, J = 12.4, 7.1 Hz, 1H), 3.02-2.89 (m, 1H), 2.78-2.65 (m, 1H), 2.65-2.52 (m, 1H), 2.33- 2.11 (m, 9H). ³¹P NMR (162 MHz, CDCl₃) δ 29.87 (s). ee: >99% Retention time: 3.652 min. |
| 81 | 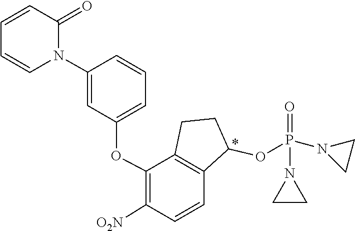 [SMILES: O=c1ccccn1-c1cccc(Oc2c([N+](=O)[O-])ccc3c2CCC3OP(=O)(N2CC2)N2CC2)c1] | LCMS (ESI): m/z 495.1 [M + H]⁺; ¹H NMR (400 MHz, CDCl₃) δ 7.89 (d, J = 8.2 Hz, 1H), 7.53 (d, J = 8.2 Hz, 1H), 7.47-7.24 (m, 3H), 7.13-7.05 (m, 1H), 6.98-6.88 (m, 2H), 6.61 (d, J = 9.2 Hz, 1H), 6.28-6.19 (m, 1H), 6.05-5.96 (m, 1H), 3.02-2.88 (m, 1H), 2.78-2.65 (m, 1H), 2.65-2.53 (m, 1H), 2.31-2.14 (m, 9H). ³¹P NMR (162 MHz, CDCl₃) δ 29.86 (s). ee: >99% Retention time: 4.841 min. |
| 82 | 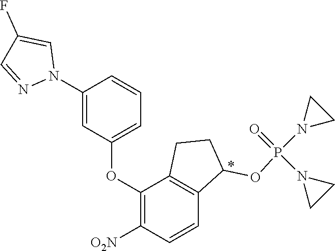 [SMILES: O=[N+]([O-])c1ccc2c(c1Oc1cccc(-n3cc(F)cn3)c1)CCC2OP(=O)(N1CC1)N1CC1] | LCMS (ESI): m/z 486.1 [M + H]⁺; ¹H NMR (400 MHz, CDCl₃) δ 7.92 (d, J = 8.2 Hz, 1H), 7.78 (d, J = 4.8 Hz, 1H), 7.56-7.52 (m, 2H), 7.36 (t, J = 8.1 Hz, 1H), 7.31-7.29 (m, 1H), 7.25 (s, 1H), 6.78-6.72 (m, 1H), 6.04-5.99 (m, 1H), 2.91-2.87 (m, 1H), 2.72-2.50 (m, 2H), 2.29-2.17 (m, 9H). ³¹P NMR (162 MHz, CDCl₃) δ 29.96 (s). ee: >99% Retention time: 2.878 min. |
| 83 | 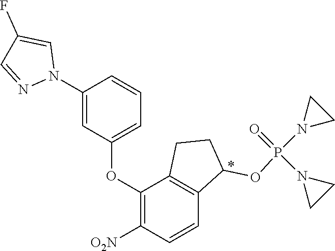 [SMILES: O=[N+]([O-])c1ccc2c(c1Oc1cccc(-n3cc(F)cn3)c1)CCC2OP(=O)(N1CC1)N1CC1] | LCMS (ESI): m/z 486.1 [M + H]⁺; ¹H NMR (400 MHz, CDCl₃) δ 7.92 (d, J = 8.2 Hz, 1H), 7.78 (d, J = 4.8 Hz, 1H), 7.58-7.52 (m, 2H), 7.36 (t, J = 8.1 Hz, 1H), 7.30-7.29 (m, 1H), 7.25 (s, 1H), 6.76-6.74 (m, 1H), 6.02-6.01 (m, 1H), 2.9-2.88 (m, 1H), 2.70-2.53 (m, 2H), 2.30-2.17 (m, 9H). ³¹P NMR (162 MHz, CDCl₃) δ 29.96 (s). ee: >99% Retention time: 3.272 min. |
| 84 | 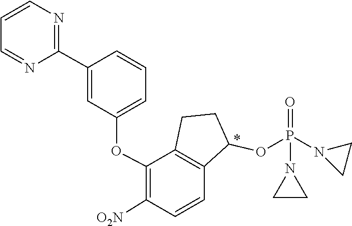 [SMILES: O=[N+]([O-])c1ccc2c(c1Oc1cccc(-c3ncccn3)c1)CCC2OP(=O)(N1CC1)N1CC1] | LCMS (ESI): m/z 480.1 [M + H]⁺; ¹H NMR (400 MHz, CDCl₃) δ 8.77 (d, J = 4.8 Hz, 2H), 8.19 (d, J = 7.8 Hz, 1H), 8.01-7.94 (m, 1H), 7.91 (d, J = 8.2 Hz, 1H), 7.52 (d, J = 8.2 Hz, 1H), 7.46-7.38 (m, 1H), 7.20-7.15 (m, 1H), 7.02-6.97 (m, 1H), 6.06-5.93 (m, 1H), 2.94-2.81 (m, 1H), 2.67-2.48 (m, 2H), 2.28-2.14 (m, 9H). ³¹P NMR (162 MHz, CDCl₃) δ 29.91 (s). ee: >99% Retention time: 2.227 min. |
| 85 | 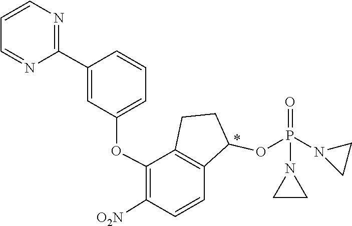 [SMILES: O=[N+]([O-])c1ccc2c(c1Oc1cccc(-c3ncccn3)c1)CCC2OP(=O)(N1CC1)N1CC1] | LCMS (ESI): m/z 480.1 [M + H]⁺; ¹H NMR (400 MHz, CDCl₃) δ 8.78 (d, J = 4.8 Hz, 2H), 8.19 (d, J = 7.9 Hz, 1H), 8.01-7.96 (m, 1H), 7.92 (d, J = 8.2 Hz, 1H), 7.55-7.48 (m, 1H), 7.46-7.40 (m, 1H), 7.21-7.17 (m, 1H), 7.02-6.97 (m, 1H), 6.04- 5.94 (m, 1H), 2.94-2.82 (m, 1H), 2.66-2.49 (m, 2H), 2.28-2.13 (m, 9H). ³¹P NMR (162 MHz, CDCl₃) δ 29.91 (s). ee: >99% Retention time: 3.044 min. |
| 86 | 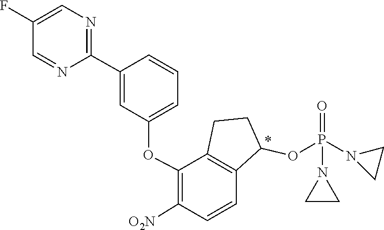 [SMILES: O=[N+]([O-])c1ccc2c(c1Oc1cccc(-c3ncc(F)cn3)c1)CCC2OP(=O)(N1CC1)N1CC1] | LCMS (ESI): m/z 498.1 [M + H]⁺; ¹H NMR (400 MHz, CDCl₃) δ 8.78 (d, J = 4.8 Hz, 2H), 8.19 (d, J = 7.9 Hz, 1H), 8.01-7.96 (m, 1H), 7.92 (d, J = 8.2 Hz, 1H), 7.55-7.48 (m, 1H), 7.46-7.40 (m, 1H), 7.21-7.17 (m, 1H), 7.02-6.97 (m, 1H), 6.04- 5.94 (m, 1H), 2.94-2.82 (m, 1H), 2.66-2.49 (m, 2H), 2.28-2.13 (m, 9H). ³¹P NMR (162 MHz, CDCl₃) δ 29.98 (s). ee: >99% Retention time: 3.849 min. |
| 87 | 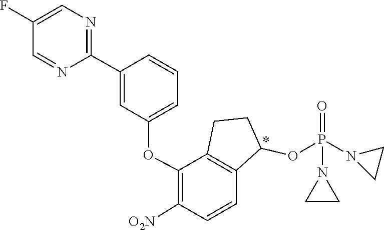 [SMILES: O=[N+]([O-])c1ccc2c(c1Oc1cccc(-c3ncc(F)cn3)c1)CCC2OP(=O)(N1CC1)N1CC1] | LCMS (ESI): m/z 498.1 [M + H]⁺; ¹H NMR (400 MHz, CDCl₃) δ 8.62 (s, 2H), 8.13 (d, J = 7.8 Hz, 1H), 7.94-7.88 (m, 2H), 7.53 (d, J = 8.2 Hz, 1H), 7.45-7.40 (m, 1H), 7.02- 6.97 (m, 1H), 6.06-5.98 (m, 1H), 2.93-2.84 (m, 1H), 2.68-2.51 (m, 2H), 2.29-2.15 (m, 9H). ³¹P NMR (162 MHz, CDCl₃) δ 29.99 (s). ee: >99% Retention time: 4.733 min. |
| 88 | 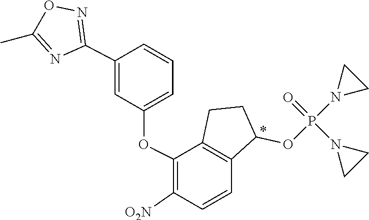 [SMILES: Cc1nc(-c2cccc(Oc3c([N+](=O)[O-])ccc4c3CCC4OP(=O)(N3CC3)N3CC3)c2)no1] | LCMS (ESI): m/z 483.1 [M + H]⁺; ¹H NMR (400 MHz, CDCl₃) δ 7.92 (d, J = 8.2 Hz, 1H), 7.80 (d, J = 7.7 Hz, 1H), 7.60-7.49 (m, 2H), 7.43 (t, J = 8.0 Hz, 1H), 7.11-6.94 (m, 1H), 6.03-5.98 (m, 1H), 2.97-2.79 (m, 1H), 2.64 (s, 3H), 2.61-2.51 (m, 2H), 2.31-2.17 (m, 9H). ³¹P NMR (162 MHz, CDCl₃) δ 29.91 (s). ee: >99% Retention time: 3.064 min. |
| 89 | 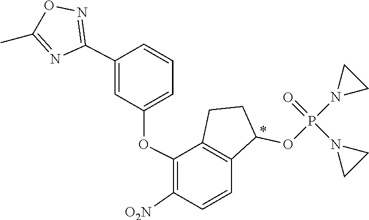 [SMILES: Cc1nc(-c2cccc(Oc3c([N+](=O)[O-])ccc4c3CCC4OP(=O)(N3CC3)N3CC3)c2)no1] | LCMS (ESI): m/z 484.1 [M + H]⁺; ¹H NMR (400 MHz, CDCl₃) δ 7.92 (d, J = 8.2 Hz, 1H), 7.80 (d, J = 7.7 Hz, 1H), 7.60-7.49 (m, 2H), 7.43 (t, J = 8.0 Hz, 1H), 7.11-6.94 (m, 1H), 6.03-5.98 (m, 1H), 2.97-2.79 (m, 1H), 2.64 (s, 3H), 2.61-2.51 (m, 2H), 2.31-2.17 (m, 9H). ³¹P NMR (162 MHz, CDCl₃) δ 29.91 (s). ee: >99% Retention time: 3.932 min. |
| 90 | 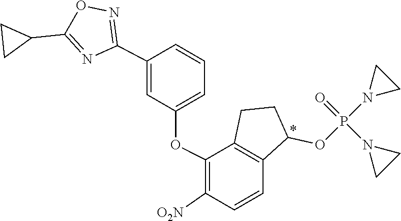 [SMILES: O=[N+]([O-])c1ccc2c(c1Oc1cccc(-c3noc(C4CC4)n3)c1)CCC2OP(=O)(N1CC1)N1CC1] | LCMS (ESI): m/z 510.1 [M + H]⁺; ¹H NMR (400 MHz, CDCl₃) δ 7.92 (d, J = 8.2 Hz, 1H), 7.77 (d, J = 7.7 Hz, 1H), 7.57-7.51 (m, 2H), 7.40 (t, J = 8.0 Hz, 1H), 7.02-6.96 (m, 1H), 6.03-5.98 (m, 1H), 2.94-2.78 (m, 1H), 2.68-2.48 (m, 2H), 2.33-2.13 (m, 10H), 1.37-1.18 (m, 4H). ³¹P NMR (162 MHz, CDCl₃) δ 29.89 (s). ee: >99% Retention time: 4.400 min. |
| 91 | 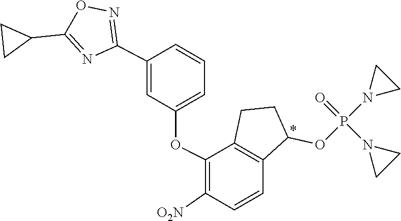 [SMILES: O=[N+]([O-])c1ccc2c(c1Oc1cccc(-c3noc(C4CC4)n3)c1)CCC2OP(=O)(N1CC1)N1CC1] | LCMS (ESI): m/z 510.1 [M + H]⁺; ¹H NMR (400 MHz, CDCl₃) δ 7.92 (d, J = 8.2 Hz, 1H), 7.77 (d, J = 7.7 Hz, 1H), 7.58-7.50 (m, 2H), 7.40 (t, J = 8.0 Hz, 1H), 7.02-6.97 (m, 1H), 6.03-5.98 (m, 1H), 2.94-2.78 (m, 1H), 2.68-2.48 (m, 2H), 2.33-2.13 (m, 10H), 1.37-1.18 (m, 4H). ³¹P NMR (162 MHz, CDCl₃) δ 9.89 (s). ee: >99% Retention time: 5.710 min. |
| 92 | 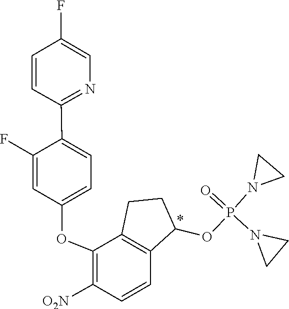 [SMILES: O=[N+]([O-])c1ccc2c(c1Oc1ccc(-c3ccc(F)cn3)c(F)c1)CCC2OP(=O)(N1CC1)N1CC1] | LCMS (ESI): m/z 515 [M + H]⁺; ¹H NMR (400 MHz, CDCl₃) δ 8.54 (d, J = 2.9 Hz, 1H), 7.99-7.84 (m, 2H), 7.79-7.69 (m, 1H), 7.57 (d, J = 8.2 Hz, 1H), 7.51-7.39 (m, 1H), 6.83-6.64 (m, 2H), 6.08-5.95 (m, 1H), 2.99-2.84 (m, 1H), 2.74-2.51 (m, 2H), 2.34-2.12 (m, 9H). ³¹P NMR (162 MHz, CDCl₃) δ 29.98 (s). ee: >95% Retention time: 3.188 min. |
| 93 | 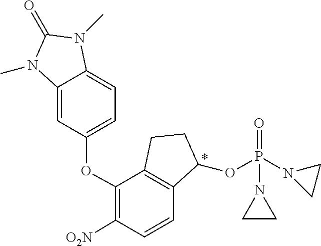 [SMILES: Cn1c(=O)n(C)c2cc(Oc3c([N+](=O)[O-])ccc4c3CCC4OP(=O)(N3CC3)N3CC3)ccc21] | LCMS (ESI): m/z 486.1 [M + H]⁺; ¹H NMR (400 MHz, CDCl₃) δ 8.54 (d, J = 2.9 Hz, 1H), 7.99-7.84 (m, 2H), 7.79-7.69 (m, 1H), 7.57 (d, J = 8.2 Hz, 1H), 7.51-7.39 (m, 1H), 6.83-6.64 (m, 2H), 6.08-5.95 (m, 1H), 2.99-2.84 (m, 1H), 2.74-2.51 (m, 2H), 2.34-2.12 (m, 9H). ³¹P NMR (162 MHz, CDCl₃) δ 30.04 (s). ee: >99% Retention time: 4.033 min. |
| 94 | 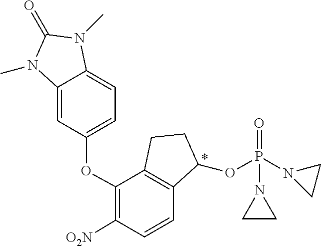 [SMILES: Cn1c(=O)n(C)c2cc(Oc3c([N+](=O)[O-])ccc4c3CCC4OP(=O)(N3CC3)N3CC3)ccc21] | LCMS (ESI): m/z 486.1 [M + H]⁺; ¹H NMR (400 MHz, CDCl₃) δ 7.87 (d, J = 8.2 Hz, 1H), 7.50 (d, J = 8.2 Hz, 1H), 6.81 (t, J = 6.7 Hz, 1H), 6.64 (d, J = 2.3 Hz, 1H), 6.55 (dd, J = 8.4, 2.3 Hz, 1H), 6.03-5.95 (m, 1H), 3.40 (s, 3H), 3.37 (s, 3H), 2.89- 2.79 (m, 1H), 2.63-2.49 (m, 2H), 2.30-2.16 (m, 9H). ³¹P NMR (162 MHz, CDCl₃) δ 30.04 (s). ee: >99% Retention time: 4.707 min. |
| 95 | 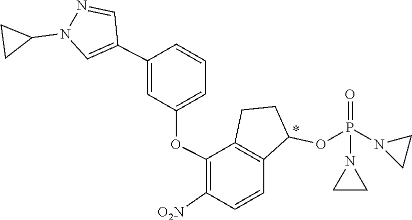 [SMILES: O=[N+]([O-])c1ccc2c(c1Oc1cccc(-c3cnn(C4CC4)c3)c1)CCC2OP(=O)(N1CC1)N1CC1] | LCMS (ESI): m/z 508.2 [M + H]⁺; ¹H NMR (400 MHz, CDCl₃) δ 7.87 (d, J = 8.2 Hz, 1H), 7.50 (d, J = 8.2 Hz, 1H), 6.81 (t, J = 6.7 Hz, 1H), 6.64 (d, J = 2.3 Hz, 1H), 6.55 (dd, J = 8.4, 2.3 Hz, 1H), 6.03-5.95 (m, 1H), 3.40 (s, 3H), 3.37 (s, 3H), 2.89- 2.79 (m, 1H), 2.63-2.49 (m, 2H), 2.30-2.16 (m, 9H). ³¹P NMR (162 MHz, CDCl₃) δ 29.96 (s). ee: >99% Retention time: 5.580 min. |
| 96 | 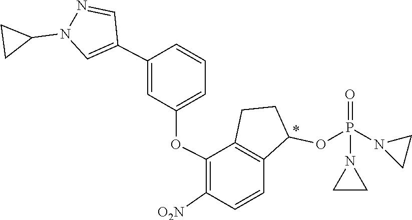 [SMILES: O=[N+]([O-])c1ccc2c(c1Oc1cccc(-c3cnn(C4CC4)c3)c1)CCC2OP(=O)(N1CC1)N1CC1] | LCMS (ESI): m/z 508.2 [M + H]⁺; ¹H NMR (400 MHz, CDCl₃) δ 7.89 (d, J = 8.2 Hz, 1H), 7.71-7.65 (m, 2H), 7.51 (d, J = 8.2 Hz, 1H), 7.26-7.23 (m, 1H), 7.20-7.16 (m, 1H), 7.04-7.01 (m, 1H), 6.68-6.62 (m, 1H), 6.04-5.96 (m, 1H), 3.66-3.58 (m, 1H), 2.93-2.83 (m, 1H), 2.67-2.48 (m, 2H), 2.29-2.16 (m, 9H), 1.18- 1.13 (m, 2H), 1.07-1.02 (m, 2H). ³¹P NMR (162 MHz, CDCl₃) δ 29.96 (s). ee: >99% Retention time: 9.647 min. |
| 97 | 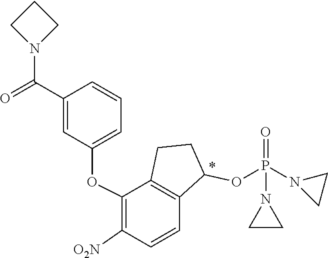 [SMILES: O=C(c1cccc(Oc2c([N+](=O)[O-])ccc3c2CCC3OP(=O)(N2CC2)N2CC2)c1)N1CCC1] | LCMS (ESI): m/z 485.1 [M + H]⁺; ¹H NMR (400 MHz, CDCl₃) δ 7.90 (d, J = 8.2 Hz, 1H), 7.53 (d, J = 8.2 Hz, 1H), 7.38-7.29 (m, 2H), 7.09-7.05 (m, 1H), 7.01 (d, J = 6.6 Hz, 1H), 6.05-5.95 (m, 1H), 4.26-4.15 (m, 4H), 2.90-2.80 (m, 1H), 2.67- 2.49 (m, 2H), 2.37-2.29 (m, 2H), 2.29-2.14 (m, 9H). ³¹P NMR (162 MHz, CDCl₃) δ 29.90 (s). ee: >99% Retention time: 4.824 min. |
| 98 | 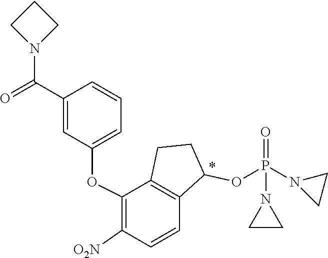 [SMILES: O=C(c1cccc(Oc2c([N+](=O)[O-])ccc3c2CCC3OP(=O)(N2CC2)N2CC2)c1)N1CCC1] | LCMS (ESI): m/z 485.1 [M + H]⁺; ¹H NMR (400 MHz, CDCl₃) δ 7.90 (d, J = 8.2 Hz, 1H), 7.53 (d, J = 8.2 Hz, 1H), 7.39-7.29 (m, 2H), 7.11-7.05 (m, 1H), 7.01 (d, J = 6.7 Hz, 1H), 6.07-5.95 (m, 1H), 4.26-4.14 (m, 4H), 2.91-2.80 (m, 1H), 2.65- 2.51 (m, 2H), 2.38-2.29 (m, 2H), 2.29-2.15 (m, 9H). ³¹P NMR (162 MHz, CDCl₃) δ 29.90 (s). ee: >99% Retention time: 6.412 min. |
| 99 | 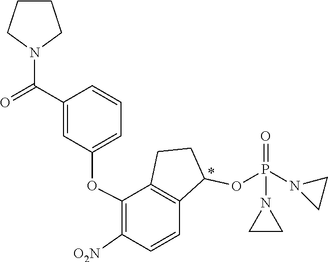 [SMILES: O=C(c1cccc(Oc2c([N+](=O)[O-])ccc3c2CCC3OP(=O)(N2CC2)N2CC2)c1)N1CCCC1] | LCMS (ESI): m/z 499.2 [M + H]⁺; ¹H NMR (400 MHz, CDCl₃) δ 7.89 (d, J = 8.2 Hz, 1H), 7.53 (d, J = 8.2 Hz, 1H), 7.34 (t, J = 7.8 Hz, 1H), 7.21 (d, J = 7.6 Hz, 1H), 6.99-6.92 (m, 2H), 6.05-5.95 (m, 1H), 3.60 (t, J = 6.8 Hz, 2H), 3.37 (t, J = 6.5 Hz, 2H), 2.92-2.82 (m, 1H), 2.67-2.51 (m, 2H), 2.29-2.18 (m, 9H), 1.97- 1.85 (m, 4H). ³¹P NMR (162 MHz, CDCl₃) δ 29.89 (s). ee: >99% Retention time: 4.289 min. |
| 100 | 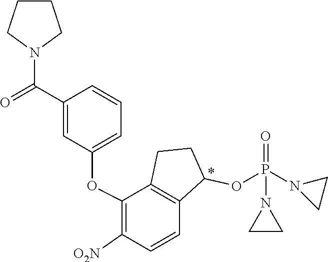 [SMILES: O=C(c1cccc(Oc2c([N+](=O)[O-])ccc3c2CCC3OP(=O)(N2CC2)N2CC2)c1)N1CCCC1] | LCMS (ESI): m/z 449.2 [M + H]⁺; ¹H NMR (400 MHz, CDCl₃) δ 7.89 (d, J = 8.2 Hz, 1H), 7.53 (d, J = 8.2 Hz, 1H), 7.35 (t, J = 7.8 Hz, 1H), 7.21 (d, J = 7.6 Hz, 1H), 6.99-6.93 (m, 2H), 6.00 (dd, J = 12.3, 7.1 Hz, 1H), 3.60 (t, J = 6.8 Hz, 2H), 3.37 (t, J = 6.5 Hz, 2H), 2.93-2.81 (m, 1H), 2.67-2.51 (m, 2H), 2.29-2.18 (m, 9H), 1.97-1.85 (m, 4H). ³¹P NMR (162 MHz, CDCl₃) δ 29.89 (s). ee: >99% Retention time: 4.654 min. |
| 101 | 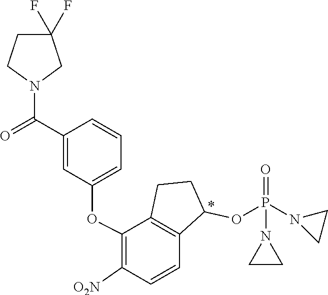 [SMILES: O=C(c1cccc(Oc2c([N+](=O)[O-])ccc3c2CCC3OP(=O)(N2CC2)N2CC2)c1)N1CCC(F)(F)C1] | LCMS (ESI): m/z 535.1 [M + H]⁺; ¹H NMR (400 MHz, CDCl₃) δ 7.83 (d, J = 8.2 Hz, 1H), 7.48 (d, J = 8.2 Hz, 1H), 7.31 (t, J = 8.3 Hz, 1H), 7.17-7.09 (m, 1H), 6.95-6.87 (m, 2H), 5.98-5.89 (m, 1H), 3.93-3.54 (m, 4H), 2.86-2.75 (m, 1H), 2.61-2.45 (m, 2H), 2.40-2.24 (m, 2H), 2.22-2.09 (m, 9H). ³¹P NMR (162 MHz, CDCl₃) δ 29.96 (s). ee: >99% Retention time: 5.742 min. |
| 102 | 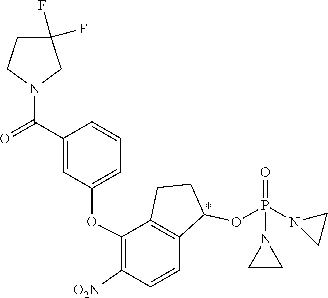 [SMILES: O=C(c1cccc(Oc2c([N+](=O)[O-])ccc3c2CCC3OP(=O)(N2CC2)N2CC2)c1)N1CCC(F)(F)C1] | LCMS (ESI): m/z 535.1 [M + H]⁺; ¹H NMR (400 MHz, CDCl₃) δ 7.83 (d, J = 8.2 Hz, 1H), 7.48 (d, J = 8.2 Hz, 1H), 7.31 (t, J = 8.3 Hz, 1H), 7.17-7.09 (m, 1H), 6.95-6.87 (m, 2H), 5.98-5.89 (m, 1H), 3.93-3.54 (m, 4H), 2.86-2.75 (m, 1H), 2.61-2.45 (m, 2H), 2.40-2.24 (m, 2H), 2.22-2.09 (m, 9H). ³¹P NMR (162 MHz, CDCl₃) δ 29.96 (s). ee: >99% Retention time: 6.231 min. |
| 103 | 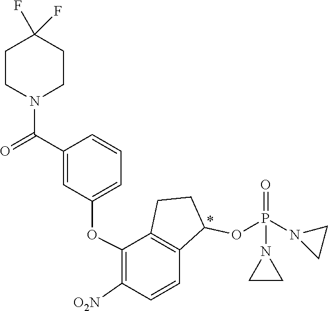 [SMILES: O=C(c1cccc(Oc2c([N+](=O)[O-])ccc3c2CCC3OP(=O)(N2CC2)N2CC2)c1)N1CCC(F)(F)CC1] | LCMS (ESI): m/z 549.2 [M + H]⁺; ¹H NMR (400 MHz, CDCl₃) δ 7.88 (d, J = 8.2 Hz, 1H), 7.54 (d, J = 8.2 Hz, 1H), 7.36 (t, J = 7.9 Hz, 1H), 7.10 (d, J = 7.6 Hz, 1H), 6.99-6.93 (m, 1H), 6.86 (s, 1H), 6.05-5.96 (m, 1H), 3.95-3.40 (m, 4H), 2.95-2.84 (m, 1H), 2.69-2.52 (m, 2H), 2.29-2.17 (m, 9H), 2.12-1.86 (m, 4H). ³¹P NMR (162 MHz, CDCl₃) δ 29.97 (s). ee: >99% Retention time: 2.527 min. |
| 104 | 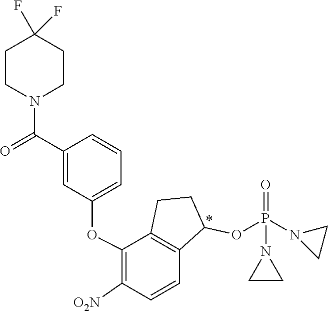 [SMILES: O=C(c1cccc(Oc2c([N+](=O)[O-])ccc3c2CCC3OP(=O)(N2CC2)N2CC2)c1)N1CCC(F)(F)CC1] | LCMS (ESI): m/z 549.2 [M + H]⁺; ¹H NMR (400 MHz, CDCl₃) δ 7.88 (d, J = 8.2 Hz, 1H), 7.53 (d, J = 8.2 Hz, 1H), 7.36 (t, J = 7.9 Hz, 1H), 7.10 (d, J = 7.6 Hz, 1H), 6.99-6.93 (m, 1H), 6.86 (s, 1H), 6.04-5.94 (m, 1H), 3.66 (d, J = 127.3 Hz, 4H), 2.98-2.83 (m, 1H), 2.71-2.50 (m, 2H), 2.29-2.17 (m, 9H), 2.11-1.88 (m, 4H). ³¹P NMR (162 MHz, CDCl₃) δ 29.97 (s). ee: >98% Retention time: 2.838 min. |
| 105 | 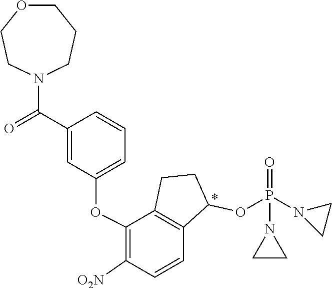 [SMILES: O=C(c1cccc(Oc2c([N+](=O)[O-])ccc3c2CCC3OP(=O)(N2CC2)N2CC2)c1)N1CCCOCC1] | LCMS (ESI): m/z 529.2 [M + H]⁺; ¹H NMR (400 MHz, CDCl₃) δ 7.89 (d, J = 8.2 Hz, 1H), 7.54 (d, J = 8.2 Hz, 1H), 7.39-7.32 (m, 1H), 7.13-7.07 (m, 1H), 6.98-6.90 (m, 1H), 6.86 (s, 1H), 6.10-5.95 (m, 1H), 3.87-3.77 (m, 4H), 3.77-3.73 (m, 1H), 3.66- 3.60 (m, 1H), 3.52-3.44 (m, 2H), 2.96-2.84 (m, 1H), 2.69-2.52 (m, 2H), 2.34-2.11 (m, 9H), 2.07-1.97 (m, 1H), 1.84-1.77 (m, 1H). ³¹P NMR (162 MHz, CDCl₃) δ 29.93 (s). ee: >99% Retention time: 3.948 min. |
| 106 | 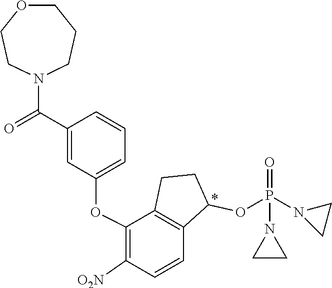 [SMILES: O=C(c1cccc(Oc2c([N+](=O)[O-])ccc3c2CCC3OP(=O)(N2CC2)N2CC2)c1)N1CCCOCC1] | LCMS (ESI): m/z 529.2 [M + H]⁺; ¹H NMR (400 MHz, CDCl₃) δ 7.89 (d, J = 8.2 Hz, 1H), 7.54 (d, J = 8.2 Hz, 1H), 7.39-7.32 (m, 1H), 7.10 (d, J = 7.5 Hz, 1H), 6.98-6.90 (m, 1H), 6.86 (s, 1H), 6.07-5.86 (m, 1H), 3.87-3.78 (m, 4H), 3.77-3.73 (m, 1H), 3.66-3.61 (m, 1H), 3.51-3.44 (m, 2H), 2.96-2.83 (m, 1H), 2.69- 2.51 (m, 2H), 2.31-2.15 (m, 9H), 2.08-1.97 (m, 1H), 1.85-1.77 (m, 1H). ³¹P NMR (162 MHz, CDCl₃) δ 29.92 (s). ee: >99% Retention time: 4.449 min. |
| 107 | 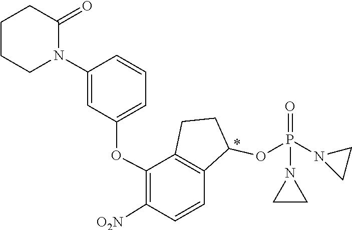 [SMILES: O=C1CCCCN1c1cccc(Oc2c([N+](=O)[O-])ccc3c2CCC3OP(=O)(N2CC2)N2CC2)c1] | LCMS (ESI): m/z 499.2 [M + H]⁺; ¹H NMR (400 MHz, CDCl₃) δ 7.87 (d, J = 8.2 Hz, 1H), 7.61-7.44 (m, 1H), 7.31 (t, J = 7.5 Hz, 1H), 6.98 (d, J = 7.8 Hz, 1H), 6.84-6.79 (m, 1H), 6.77-6.74 (m, 1H), 6.02-5.97 (m, 1H), 3.72-3.51 (m, 2H), 2.94- 2.86 (m, 1H), 2.77-2.44 (m, 4H), 2.35-2.08 (m, 9H), 2.04-1.85 (m, 4H). ³¹P NMR (162 MHz, CDCl₃) δ 29.90 (s). ee: >99% Retention time: 4.414 min. |
| 108 | 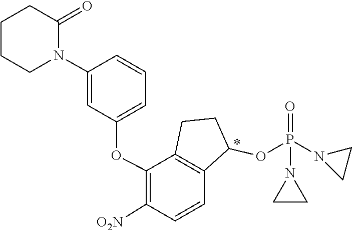 [SMILES: O=C1CCCCN1c1cccc(Oc2c([N+](=O)[O-])ccc3c2CCC3OP(=O)(N2CC2)N2CC2)c1] | LCMS (ESI): m/z 499.2 [M + H]⁺; ¹H NMR (400 MHz, CDCl₃) δ 7.87 (d, J = 8.2 Hz, 1H), 7.61-7.44 (m, 1H), 7.31 (t, J = 7.5 Hz, 1H), 6.98 (d, J = 7.8 Hz, 1H), 6.84-6.79 (m, 1H), 6.77-6.74 (m, 1H), 6.02-5.97 (m, 1H), 3.72-3.51 (m, 2H), 2.94- 2.86 (m, 1H), 2.77-2.44 (m, 4H), 2.35-2.08 (m, 9H), 2.04-1.85 (m, 4H). ³¹P NMR (162 MHz, CDCl₃) δ 29.90 (s). ee: >99% Retention time: 5.286 min. |
| 109 | 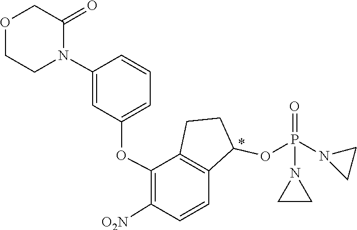 [SMILES: O=C1COCCN1c1cccc(Oc2c([N+](=O)[O-])ccc3c2CCC3OP(=O)(N2CC2)N2CC2)c1] | LCMS (ESI): m/z 501.1 [M + H]⁺; ¹H NMR (400 MHz, CDCl₃) δ 7.88 (d, J = 8.2 Hz, 1H), 7.52 (d, J = 8.1 Hz, 1H), 7.34 (t, J = 8.2 Hz, 1H), 7.08-7.03 (m, 1H), 6.95-6.89 (m, 1H), 6.81-6.75 (m, 1H), 6.06-5.97 (m, 1H), 4.30 (s, 2H), 4.01 (t, J = 5.0 Hz, 2H), 3.80-3.71 (m, 2H), 2.96-2.87 (m, 1H), 2.72-2.54 (m, 2H), 2.31- 2.16 (m, 9H). ³¹P NMR (162 MHz, CDCl₃) δ 29.94 (s). ee: >99% Retention time: 5.180 min. |
| 110 | 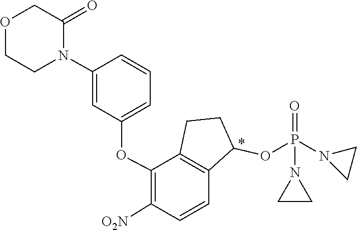 [SMILES: O=C1COCCN1c1cccc(Oc2c([N+](=O)[O-])ccc3c2CCC3OP(=O)(N2CC2)N2CC2)c1] | LCMS (ESI): m/z 501.1 [M + H]⁺; ¹H NMR (400 MHz, CDCl₃) δ 7.88 (d, J = 8.2 Hz, 1H), 7.52 (d, J = 8.1 Hz, 1H), 7.34 (t, J = 8.2 Hz, 1H), 7.09-7.03 (m, 1H), 6.95-6.90 (m, 1H), 6.81-6.74 (m, 1H), 6.05-5.96 (m, 1H), 4.30 (s, 2H), 4.01 (t, J = 5.0 Hz, 2H), 3.79-3.71 (m, 2H), 2.96-2.85 (m, 1H), 2.71-2.53 (m, 2H), 2.33- 2.15 (m, 9H). ³¹P NMR (162 MHz, CDCl₃) δ 29.94 (s). ee: 97% Retention time: 5.933 min. |
| 111 | 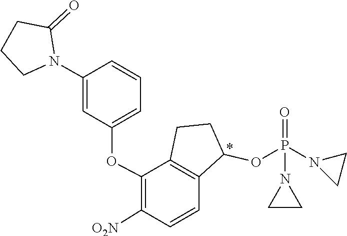 [SMILES: O=C1CCCN1c1cccc(Oc2c([N+](=O)[O-])ccc3c2CCC3OP(=O)(N2CC2)N2CC2)c1] | LCMS (ESI): m/z 485.2 [M + H]⁺; ¹H NMR (400 MHz, CDCl₃) δ 7.88 (d, J = 8.2 Hz, 1H), 7.51 (t, J = 5.7 Hz, 1H), 7.38-7.36 (m, 1H), 7.29-7.27 (m, 2H), 6.64-6.57 (m, 1H), 6.03-5.98 (m, 1H), 3.83-3.81 (m, 2H), 2.88-2.86 (m, 1H), 2.70-2.52 (m, 4H), 2.29-2.11 (m, 11H). ³¹P NMR (162 MHz, CDCl₃) δ 29.90 (s). ee: >99% Retention time: 6.743 min. |
| 112 | 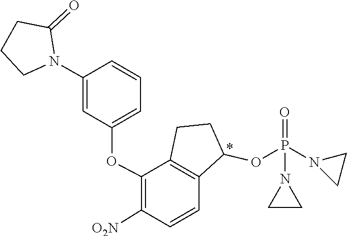 [SMILES: O=C1CCCN1c1cccc(Oc2c([N+](=O)[O-])ccc3c2CCC3OP(=O)(N2CC2)N2CC2)c1] | LCMS (ESI): m/z 485.1 [M + H]⁺; ¹H NMR (400 MHz, CDCl₃) δ 7.88 (d, J = 8.2 Hz, 1H), 7.51 (t, J = 5.7 Hz, 1H), 7.38-7.36 (m, 1H), 7.29-7.27 (m, 2H), 6.64-6.57 (m, 1H), 6.03-5.98 (m, 1H), 3.83-3.81 (m, 2H), 2.88-2.86 (m, 1H), 2.70-2.52 (m, 4H), 2.29-2.11 (m, 11H). ³¹P NMR (162 MHz, CDCl₃) δ 29.90 (s). ee: >99% Retention time: 9.686 min. |
| 113 | 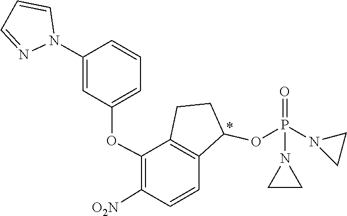 [SMILES: O=[N+]([O-])c1ccc2c(c1Oc1cccc(-n3cccn3)c1)CCC2OP(=O)(N1CC1)N1CC1] | LCMS (ESI): m/z 468.1 [M + H]⁺; ¹H NMR (400 MHz, CDCl₃) δ 7.94-7.89 (m, 2H), 7.72-7.67 (m, 1H), 7.54 (d, J = 8.2 Hz, 1H), 7.41-7.35 (m, 2H), 7.35-7.31 (m, 1H), 6.77- 6.72 (m, 1H), 6.47-6.44 (m, 1H), 6.05-5.97 (m, 1H), 2.98-2.86 (m, 1H), 2.70-2.53 (m, 2H), 2.28-2.18 (m, 9H). ³¹P NMR (162 MHz, CDCl₃) δ 29.91 (s). ee: >99% Retention time: 1.873 min. |
| 114 | 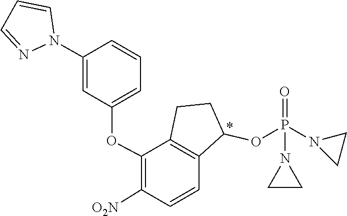 [SMILES: O=[N+]([O-])c1ccc2c(c1Oc1cccc(-n3cccn3)c1)CCC2OP(=O)(N1CC1)N1CC1] | LCMS (ESI): m/z 468.1 [M + H]⁺; ¹H NMR (400 MHz, CDCl₃) δ 7.96-7.87 (m, 2H), 7.73-7.67 (m, 1H), 7.54 (d, J = 8.2 Hz, 1H), 7.42-7.35 (m, 2H), 7.34-7.31 (m, 1H), 6.78- 6.71 (m, 1H), 6.49-6.43 (m, 1H), 6.05-5.96 (m, 1H), 2.97-2.86 (m, 1H), 2.69-2.54 (m, 2H), 2.28-2.18 (m, 9H). ³¹P NMR (162 MHz, CDCl₃) δ 29.91 (s). ee: >99% Retention time: 2.466 min. |
| 115 | 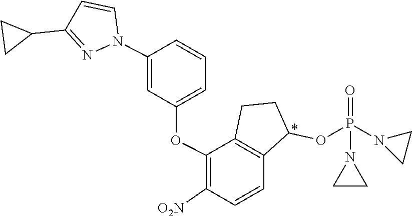 [SMILES: O=[N+]([O-])c1ccc2c(c1Oc1cccc(-n3ccc(C4CC4)n3)c1)CCC2OP(=O)(N1CC1)N1CC1] | LCMS (ESI): m/z 508.2 [M + H]⁺; ¹H NMR (400 MHz, CDCl₃) δ 7.91 (d, J = 8.2 Hz, 1H), 7.75 (d, J = 2.5 Hz, 1H), 7.53 (d, J = 7.9 Hz, 1H), 7.35-7.29 (m, 3H), 6.68-6.61 (m, 1H), 6.09 (d, J = 2.4 Hz, 1H), 6.03-5.98 (m, 1H), 2.93-2.86 (m, 1H), 2.70-2.52 (m, 2H), 2.29-2.18 (m, 9H), 2.04-1.98 (m, 1H), 0.99-0.93 (m, 2H), 0.81-0.76 (m, 2H). ³¹P NMR (162 MHz, CDCl₃) δ 29.92 (s). ee: >99% Retention time: 3.633 min. |
| 116 | 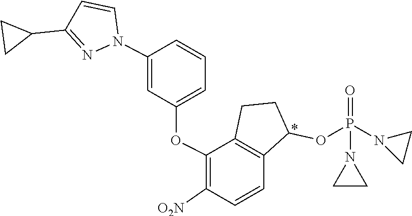 [SMILES: O=[N+]([O-])c1ccc2c(c1Oc1cccc(-n3ccc(C4CC4)n3)c1)CCC2OP(=O)(N1CC1)N1CC1] | LCMS (ESI): m/z 508.2 [M + H]⁺; ¹H NMR (400 MHz, CDCl₃) δ 7.91 (d, J = 8.2 Hz, 1H), 7.75 (d, J = 2.5 Hz, 1H), 7.53 (d, J = 7.9 Hz, 1H), 7.35-7.29 (m, 3H), 6.68-6.62 (m, 1H), 6.09 (d, J = 2.4 Hz, 1H), 6.03-5.98 (m, 1H), 2.94-2.83 (m, 1H), 2.69-2.52 (m, 2H), 2.29-2.18 (m, 9H), 2.06-1.97 (m, 1H), 0.99-0.93 (m, 2H), 0.81-0.76 (m, 2H). ee: >99% Retention time: 4.157 min. |
| 117 | 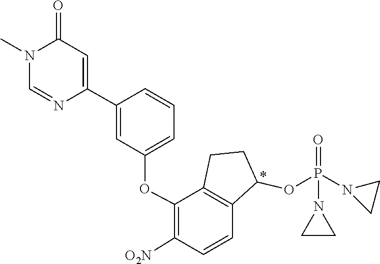 [SMILES: Cn1cnc(-c2cccc(Oc3c([N+](=O)[O-])ccc4c3CCC4OP(=O)(N3CC3)N3CC3)c2)cc1=O] | LCMS (ESI): m/z 510.1 [M + H]⁺; ¹H NMR (400 MHz, CDCl₃) δ 8.16 (s, 1H), 7.92 (d, J = 8.2 Hz, 1H), 7.65 (d, J = 7.9 Hz, 1H), 7.56-7.50 (m, 1H), 7.50-7.45 (m, 1H), 7.43- 7.36 (m, 1H), 7.01-6.93 (m, 1H), 6.82-6.76 (m, 1H), 6.06-5.96 (m, 1H), 3.55 (s, 3H), 2.94-2.81 (m, 1H), 2.66-2.50 (m, 2H), 2.29-2.14 (m, 9H). ³¹P NMR (162 MHz, CDCl₃) δ 30.00 (s). ee: >99% Retention time: 5.843 min. |
| 118 | 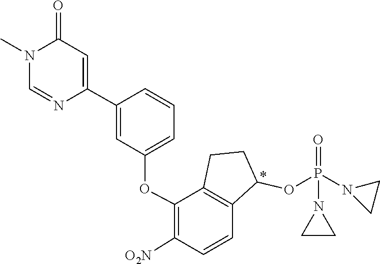 [SMILES: Cn1cnc(-c2cccc(Oc3c([N+](=O)[O-])ccc4c3CCC4OP(=O)(N3CC3)N3CC3)c2)cc1=O] | LCMS (ESI): m/z 510.1 [M + H]⁺; ¹H NMR (400 MHz, CDCl₃) δ 8.16 (s, 1H), 7.92 (d, J = 8.2 Hz, 1H), 7.65 (d, J = 7.7 Hz, 1H), 7.54 (d, J = 8.3 Hz, 1H), 7.49-7.46 (m, 1H), 7.42-7.38 (m, 1H), 6.98-6.94 (m, 1H), 6.82-6.79 (m, 1H), 6.07-5.96 (m, 1H), 3.55 (s, 3H), 2.91-2.84 (m, 1H), 2.64-2.53 (m, 2H), 2.28-2.17 (m, 9H). ³¹P NMR (162 MHz, CDCl₃) δ 29.99 (s). ee: >99% Retention time: 6.868 min. |
| 119 | 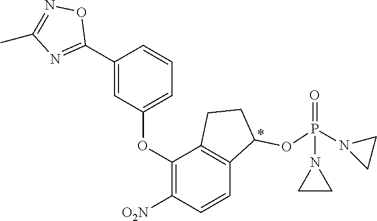 [SMILES: Cc1noc(-c2cccc(Oc3c([N+](=O)[O-])ccc4c3CCC4OP(=O)(N3CC3)N3CC3)c2)n1] | LCMS (ESI): m/z 484.1 [M + H]⁺; ¹H NMR (400 MHz, CDCl₃) δ 7.93 (d, J = 8.2 Hz, 1H), 7.84 (d, J = 7.7 Hz, 1H), 7.59-7.54 (m, 2H), 7.47 (t, J = 8.0 Hz, 1H), 7.11 (dd, J = 8.3, 2.5 Hz, 1H), 6.05-5.98 (m, 1H), 2.94-2.81 (m, 1H), 2.68-2.52 (m, 2H), 2.44 (s, 3H), 2.29-2.17 (m, 9H). ³¹P NMR (162 MHz, CDCl₃) δ 29.94 (s). ee: >99% Retention time: 3.029 min. |
| 120 | 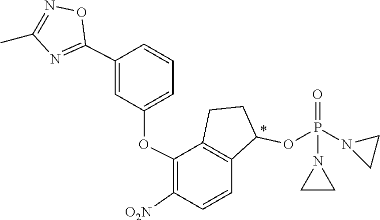 [SMILES: Cc1noc(-c2cccc(Oc3c([N+](=O)[O-])ccc4c3CCC4OP(=O)(N3CC3)N3CC3)c2)n1] | LCMS (ESI): m/z 484.1 [M + H]⁺; ¹H NMR (400 MHz, CDCl₃) δ 7.93 (d, J = 8.2 Hz, 1H), 7.83 (d, J = 7.7 Hz, 1H), 7.59-7.54 (m, 2H), 7.47 (t, J = 8.0 Hz, 1H), 7.14-7.07 (m, 1H), 6.04-5.98 (m, 1H), 2.93-2.82 (m, 1H), 2.67-2.54 (m, 2H), 2.44 (s, 3H), 2.29-2.15 (m, 9H). ³¹P NMR (162 MHz, CDCl₃) δ 29.94 (s). ee: >99% Retention time: 3.650 min. |
| 121 | 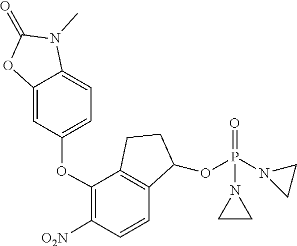 [SMILES: Cn1c(=O)oc2cc(Oc3c([N+](=O)[O-])ccc4c3CCC4OP(=O)(N3CC3)N3CC3)ccc21] | LCMS (ESI): m/z 473.0 [M + H]⁺; ¹H NMR (400 MHz, CDCl₃) δ 7.89 (d, J = 8.2 Hz, 1H), 7.53 (d, J = 8.2 Hz, 1H), 6.87-6.83 (m, 2H), 6.75-6.71 (m, 1H), 6.05-5.96 (m, 1H), 3.39 (s, 3H), 2.89-2.80 (m, 1H), 2.64-2.54 (m, 2H), 2.28-2.19 (m, 9H). ³¹P NMR (162 MHz, CDCl₃) δ 29.99 (s). |
| 122 | 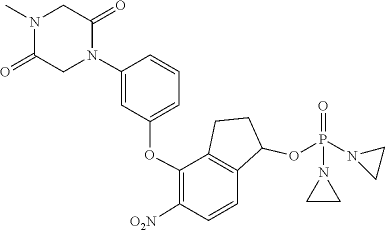 [SMILES: CN1CC(=O)N(c2cccc(Oc3c([N+](=O)[O-])ccc4c3CCC4OP(=O)(N3CC3)N3CC3)c2)CC1=O] | LCMS (ESI): m/z 528.2 [M + H]⁺; ¹H NMR (400 MHz, CDCl₃) δ 7.89 (d, J = 8.2 Hz, 1H), 7.53 (d, J = 8.2 Hz, 1H), 7.36 (t, J = 8.2 Hz, 1H), 7.01 (d, J = 7.9 Hz, 1H), 6.88-6.80 (m, 2H), 6.05-5.98 (m, 1H), 4.33 (s, 2H), 4.12 (s, 2H), 3.04 (s, 3H), 2.94-2.85 (m, 1H), 2.70-2.52 (m, 2H), 2.29-2.17 (m, 9H). ³¹P NMR (162 MHz, CDCl₃) δ 30.02 (s). |
| 123 | 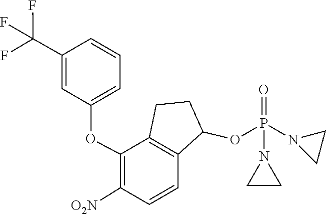 [SMILES: O=[N+]([O-])c1ccc2c(c1Oc1cccc(C(F)(F)F)c1)CCC2OP(=O)(N1CC1)N1CC1] | LCMS (ESI): m/z 939.1 [2M + H]⁺; ¹H NMR (400 MHz, CDCl₃) δ 7.93 (d, J = 8.2 Hz, 1H), 7.57 (d, J = 8.2 Hz, 1H), 7.47-7.40 (m, 1H), 7.35 (d, J = 7.7 Hz, 1H), 7.14 (s, 1H), 7.04-6.98 (m, 1H), 6.07-5.95 (m, 1H), 2.91-2.80 (m, 1H), 2.66-2.51 (m, 2H), 2.33-2.14 (m, 9H). ³¹P NMR (162 MHz, CDCl₃) δ 29.97 (s). ¹⁹F NMR (376 MHz, CDCl₃) δ −62.70 (s). |
| 124 | 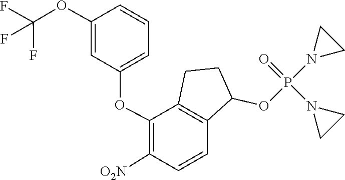 [SMILES: O=[N+]([O-])c1ccc2c(c1Oc1cccc(OC(F)(F)F)c1)CCC2OP(=O)(N1CC1)N1CC1] | LCMS (ESI): m/z 508.1 [M + Na]⁺; ¹H NMR (400 MHz, CDCl₃) δ 7.92 (d, J = 8.2 Hz, 1H), 7.56 (d, J = 8.2 Hz, 1H), 7.32 (t, J = 8.2 Hz, 1H), 6.98-6.93 (m, 1H), 6.82-6.73 (m, 2H), 6.04-5.98 (m, 1H), 2.94-2.81 (m, 1H), 2.68-2.52 (m, 2H), 2.33-2.13 (m, 9H). ³¹P NMR (162 MHz, CDCl₃) δ 29.96 (s). ¹⁹F NMR (376 MHz, CDCl₃) δ −57.84 (s), −57.84 (s). |
| 125 | 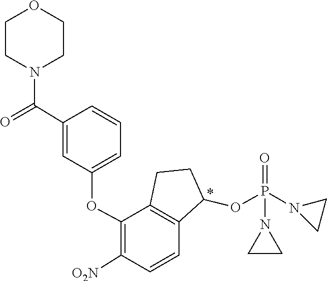 [SMILES: O=C(c1cccc(Oc2c([N+](=O)[O-])ccc3c2CCC3OP(=O)(N2CC2)N2CC2)c1)N1CCOCC1] | LCMS (ESI): m/z 515.15 [M + H]+ ¹H NMR (400 MHz, Chloroform-d) δ 7.89 (d, J = 8.2 Hz, 1H), 7.54 (d, J = 8.2 Hz, 1H), 7.36 (t, J = 7.9 Hz, 1H), 7.11 (dt, J = 7.5, 1.2 Hz, 1H), 6.95 (dd, J = 8.2, 2.6 Hz, 1H), 6.87 (dd, J = 2.7, 1.4 Hz, 1H), 6.06- 5.94 (m, 1H), 4.01-3.24 (m, 8H), 2.98-2.78 (m, 1H), 2.77-2.50 (m, 2H), 2.41-2.13 (m, 9H). ³¹P NMR (162 MHz, Chloroform-d) δ 23.29. |
| 126 | 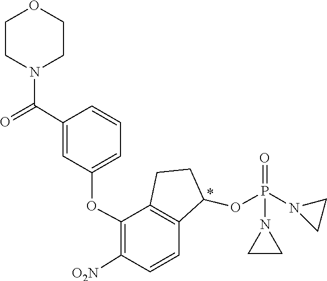 [SMILES: O=C(c1cccc(Oc2c([N+](=O)[O-])ccc3c2CCC3OP(=O)(N2CC2)N2CC2)c1)N1CCOCC1] | LCMS (ESI): m/z 515.20 [M + H]+ ¹H NMR (400 MHz, Chloroform-d) δ 7.89 (d, J = 8.2 Hz, 1H), 7.54 (d, J = 8.2 Hz, 1H), 7.36 (t, J = 7.9 Hz, 1H), 7.11 (dt, J = 7.5, 1.2 Hz, 1H), 6.95 (ddd, J = 8.3, 2.6, 1.0 Hz, 1H), 6.90-6.84 (m, 1H), 6.06-5.96 (m, 1H), 4.00-3.21 (m, 8H), 2.95-2.82 (m, 1H), 2.70-2.51 (m, 2H), 2.32- 2.15 (m, 9H). ³¹P NMR (162 MHz, Chloroform-d) δ 23.29. |

TABLE 3
Characterization of compounds (see General Procedure 6)

| Com- | | |
|---|---|---|
| pound | | |
| No. | Structure | LCMS; ¹H NMR (ppm); Retention time |
| 127 | 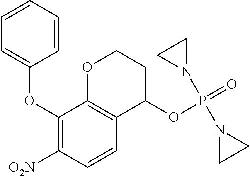 [SMILES: O=[N+]([O-])c1ccc2c(c1Oc1ccccc1)OCCC2OP(=O)(N1CC1)N1CC1] | LCMS(ESI): m/z 440.0[M + Na]⁺; ¹H NMR(400 MHz, CDCl₃) δ 7.55-7.46 (m, 2H), 7.32-7.27 (m, 2H), 7.05 (t, J = 6.9 Hz, 1H), 6.95-6.81 (m, 2H), 5.75-5.60 (m, 1H), 4.38- 4.19 (m, 2H), 2.36-2.17 (m, 10H). ³¹P NMR(162 MHz, CDCl₃) δ 29.99 (s). |
| 128 | 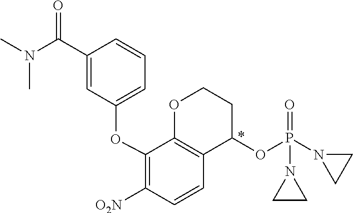 [SMILES: CN(C)C(=O)c1cccc(Oc2c([N+](=O)[O-])ccc3c2OCCC3OP(=O)(N2CC2)N2CC2)c1] | LCMS(ESI): m/z 489.2[M + H]⁺; ¹H NMR (400 MHz, CDCl₃) δ 7.56-7.43 (m, 2H), 7.33 (t, J = 7.9 Hz, 1H), 7.10 (d, J = 7.5 Hz, 1H), 6.95 (dd, J = 8.3, 2.5 Hz, 1H), 6.85 (s, 1H), 5.73- 5.57 (m, 1H), 4.38-4.16 (m, 2H), 3.08 (s, 3H), 2.95 (s, 3H), 2.40-2.14 (m, 10H). ³¹P NMR (162 MHz, CDCl₃) δ 29.93 (s). ee: >99% Retention time: 1.997 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 129 | 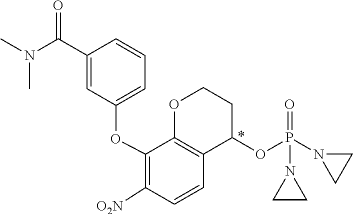 [SMILES: CN(C)C(=O)c1cccc(Oc2c([N+](=O)[O-])ccc3c2OCCC3OP(=O)(N2CC2)N2CC2)c1] | LCMS(ESI): m/z 489.3[M + H]⁺; ¹H NMR (400 MHz, CDCl₃) δ 7.55-7.45 (m, 2H), 7.33 (t, J = 7.9 Hz, 1H), 7.10 (d, J = 7.6 Hz, 1H), 6.95 (dd, J = 8.2, 2.4 Hz, 1H), 6.85 (s, 1H), 5.72-5.55 (m, 1H), 4.39-4.12 (m, 2H), 3.08 (s, 3H), 2.95 (s, 3H), 2.39-2.06 (m, 10H). ³¹P NMR (162 MHz, CDCl₃) δ 29.94 (s). ee: 99% Retention time: 2.602 min; Mobile phase: A for CO₂ and B for methanol (0.05% DEA); Gradient: 8 min @ 40% B; Flow rate: 2.5 mL/min; Column temperature: 40° C. |
| 130 | 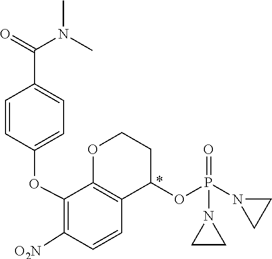 [SMILES: CN(C)C(=O)c1ccc(Oc2c([N+](=O)[O-])ccc3c2OCCC3OP(=O)(N2CC2)N2CC2)cc1] | LCMS(ESI): m/z 489.2[M + H]⁺; ¹H NMR (400 MHz, CDCl₃) δ 7.51 (s, 2H), 7.38 (d, J = 8.6 Hz, 2H), 6.87 (d, J = 8.6 Hz, 2H), 5.73-5.59 (m, 1H), 4.41-4.14 (m, 2H), 3.08 (s, 3H), 3.04 (s, 3H), 2.39-2.08 (m, 10H). ³¹P NMR (162 MHz, CDCl₃) δ 30.02 (s). ee: >99% Retention time: 3.008 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 131 | 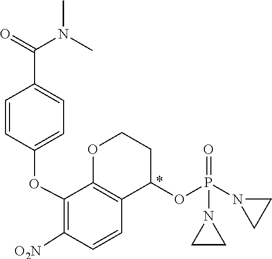 [SMILES: CN(C)C(=O)c1ccc(Oc2c([N+](=O)[O-])ccc3c2OCCC3OP(=O)(N2CC2)N2CC2)cc1] | LCMS(ESI): m/z 489.2[M + H]⁺; ¹H NMR (400 MHz, CDCl₃) δ 7.52 (d, J = 8.0 Hz, 2H), 7.42-7.34 (m, 2H), 6.93-6.80 (m, 2H), 5.73-5.58 (m, 1H), 4.38-4.14 (m, 2H), 3.08 (s, 3H), 3.04 (s, 3H), 2.34-2.15 (m, 10H). ³¹P NMR (162 MHz, CDCl₃) δ 30.02 (s). ee: 99.4% Retention time: 4.757 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 132 | 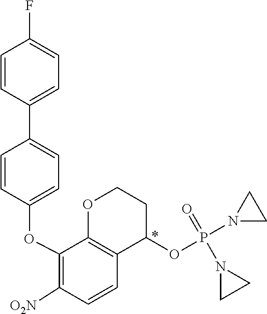 [SMILES: O=[N+]([O-])c1ccc2c(c1Oc1ccc(-c3ccc(F)cc3)cc1)OCCC2OP(=O)(N1CC1)N1CC1] | LCMS(EST): m/z 512.2[M + H]⁺; ¹H NMR (400 MHz, CDCl₃) δ 7.56-7.42 (m, 6H), 7.10 (t, J = 8.0 Hz, 2H), 6.92 (d, J = 7.7 Hz, 2H), 5.75-5.64 (m, 1H), 4.41-4.19 (m, 2H), 2.43-2.13 (m, 10H). ³¹P NMR (162 MHz, CDCl₃) δ 30.03 (s). ¹⁹F NMR (376 MHz, CDCl₃) δ −116.05-−116.44 (m) ee: 97% Retention time: 4.325 min; Column: ChiralPak IH, 100 × 4.6 mm I.D., 5 μm; Mobile phase: A for CO₂ and B for ethanol (0.05% DEA); Gradient: 8 min @ 20% B; Flow rate: 2.5 mL/min; Column temperature: 40° C. |
| 133 | 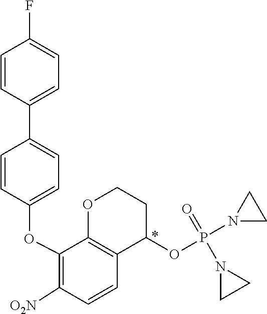 [SMILES: O=[N+]([O-])c1ccc2c(c1Oc1ccc(-c3ccc(F)cc3)cc1)OCCC2OP(=O)(N1CC1)N1CC1] | LCMS(ESI): m/z 512[M + H]⁺; ¹H NMR (400 MHz, CDCl₃) δ 7.52-7.43 (m, 6H), 7.10 (t, J = 8.0 Hz, 2H), 6.92 (d, J = 7.8 Hz, 2H), 5.80-5.56 (m, 1H), 4.39-4.23 (m, 2H), 2.47-2.08 (m, 10H). ³¹P NMR (162 MHz, CDCl₃) δ 30.03 (s). ¹⁹F NMR (376 MHz, CDCl₃) δ −116.27 (s). ee: 98% Retention time: 4.995 min; Column: ChiralPak IH, 100 × 4.6 mm I.D., 5μm; Mobile phase: A for CO₂ and B for ethanol (0.05% DEA); Gradient: 8 min @ 20% B; Flow rate: 2.5 mL/min; Column temperature: 40° C. |
| 134 | 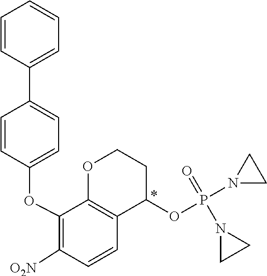 [SMILES: O=[N+]([O-])c1ccc2c(c1Oc1ccc(-c3ccccc3)cc1)OCCC2OP(=O)(N1CC1)N1CC1] | LCMS(ESI): m/z 494.3[M + H]⁺; ¹H NMR (400 MHz, CDCl₃) δ 7.58-7.47 (m, 6H), 7.45-7.37 (m, 2H), 7.34-7.28 (m, 1H), 6.96-6.88 (m, 2H), 5.73-5.60 (m, 1H), 4.43-4.14 (m, 2H), 2.42-2.08 (m, 10H). ³¹P NMR (162 MHz, CDCl₃) δ 30.01 (s). ee: 99% Retention time: 6.085 min; Column: ChiralCel OJ, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 135 | 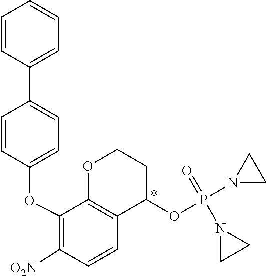 [SMILES: O=[N+]([O-])c1ccc2c(c1Oc1ccc(-c3ccccc3)cc1)OCCC2OP(=O)(N1CC1)N1CC1] | LCMS(ESI): m/z 494.2[M + H]⁺; ¹H NMR (400 MHz, CDCl₃) δ 7.58-7.47 (m, 6H), 7.45-7.38 (m, 2H), 7.35-7.28 (m, 1H), 6.96-6.89 (m, 2H), 5.72-5.63 (m, 1H), 4.43-4.18 (m, 2H), 2.40-2.13 (m, 10H). ³¹P NMR (162 MHz, CDCl₃) δ 30.01 (s). ee: >99% Retention time: 7.744 min; Column: ChiralCel OJ, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 136 | 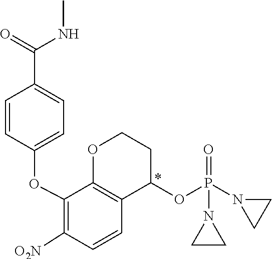 [SMILES: CNC(=O)c1ccc(Oc2c([N+](=O)[O-])ccc3c2OCCC3OP(=O)(N2CC2)N2CC2)cc1] | LCMS(ESI): m/z 475.1[M + H]⁺; ¹H NMR (400 MHz, CDCl₃) δ 7.72-7.58 (m, 2H), 7.44 (s, 2H), 6.91- 6.68 (m, 2H), 6.16-5.92 (m, 1H), 5.66-5.51 (m, 1H), 4.33-4.06 (m, 2H), 2.93 (d, J = 4.8 Hz, 3H), 2.35-2.02 (m, 10H). ³¹P NMR (162 MHz, CDCl₃) δ 30.04 (s). ee: >99% Retention time: 3.002 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 137 | 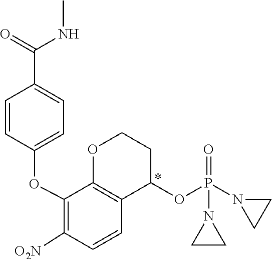 [SMILES: CNC(=O)c1ccc(Oc2c([N+](=O)[O-])ccc3c2OCCC3OP(=O)(N2CC2)N2CC2)cc1] | LCMS(ESI): m/z 475.1[M + H]⁺; ¹H NMR (400 MHz, CDCl₃) δ 7.75-7.56 (m, 2H), 7.44 (s, 2H), 6.93- 6.65 (m, 2H), 6.22-5.99 (m, 1H), 5.66-5.50 (m, 1H), 4.34- 4.03 (m, 2H), 2.92 (d, J = 4.8 Hz, 3H), 2.33-2.03 (m, 10H). ³¹P NMR (162 MHz, CDCl₃) δ 30.03 (s). ee: 99% Retention time: 3.885 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 138 | 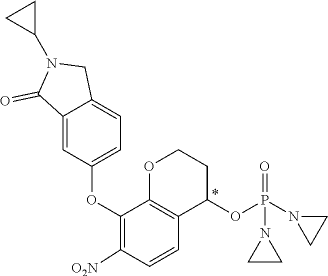 [SMILES: O=C1c2cc(Oc3c([N+](=O)[O-])ccc4c3OCCC4OP(=O)(N3CC3)N3CC3)ccc2CN1C1CC1] | LCMS(ESI): m/z 513.1 [M + H]⁺; ¹H NMR (400 MHz, CDCl₃) δ 7.52 (s, 2H), 7.38-7.33 (m, 1H), 7.26- 7.22 (m, 1H), 7.14-7.06 (m, 1H), 5.70-5.62 (m, 1H), 4.28 (s, 3H), 4.26- 4.21 (m, 1H), 2.98-2.87 (m, 1H), 2.29-2.18 (m, 10H), 0.92-0.85 (m, 4H) ³¹P NMR (162 MHz, CDCl₃) δ 29.74 (s). ee: >99% Retention time: 4.724 min; Column: ChiralPak AD, 250 × 4.6 mm I.D., 5 um, Mobile phase: A for CO₂ and B for methanol (0.05% DEA), Gradient: 8 min @B 40%, Flow rate: 2.0 mL/min, Back pressure: 100 bar, Column temperature: 35° C. |
| 139 | 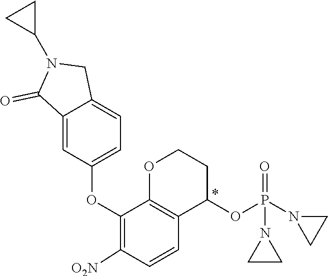 [SMILES: O=C1c2cc(Oc3c([N+](=O)[O-])ccc4c3OCCC4OP(=O)(N3CC3)N3CC3)ccc2CN1C1CC1] | LCMS(ESI): m/z 513.1 [M + H]⁺; ¹H NMR (400 MHz, CDCl₃) δ 7.54-7.50 (m, 2H), 7.39-7.34 (m, 1H), 7.26-7.23 (m, 1H), 7.11-7.04 (m, 1H), 5.71-5.59 (m, 1H), 4.28 (s, 3H), 4.25-4.20 (m, 1H), 2.96-2.88 (m, 1H), 2.29-2.19 (m, 10H), 0.97-0.85 (m, 4H). ³¹P NMR (162 MHz, CDCl₃) δ 29.74 (s). ee: >99% Retention time: 5.854 min; Column: ChiralPak AD, 250 × 4.6 mm I.D., 5 um, Mobile phase: A for CO₂ and B for methanol (0.05% DEA), Gradient: 8 min @B 40%, Flow rate: 2.0 mL/min, Back pressure: 100 bar, Column temperature: 35° C. |

TABLE 4
Characterization of compounds (see General Procedure 9)

| Com- | | |
|---|---|---|
| pound | | |
| No. | Structure | LCMS; ¹H NMR (ppm); Retention time |
| 140 | 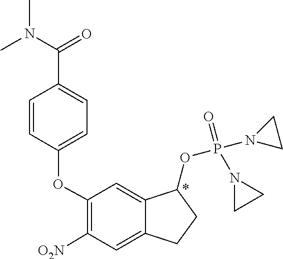 [SMILES: CN(C)C(=O)c1ccc(Oc2cc3c(cc2[N+](=O)[O-])CCC3OP(=O)(N2CC2)N2CC2)cc1] | LCMS(ESI): m/z 473.2[M + H]⁺; ¹H NMR (400 MHz, CDCl₃) δ 7.83 (s, 1H), 7.47-7.39 (m, 2H), 7.05- 6.98 (m, 2H), 7.26 (s, 1H), 5.95-5.91 (m, 1H), 3.22-3.13 (m, 1H), 3.12- 2.99 (m, 6H), 2.96-2.86 (m, 1H), 2.70-2.56 (m, 1H), 2.38-2.25 (m, 1H), 2.22-2.04 (m, 8H). ³¹P NMR (162 MHz, CDCl₃) δ 30.01 (s). ee: >99% Retention time: 4.437 min; Column: ChiralPak IA, 250 × 4.6 mm I.D., 5 μm, Mobile phase: A for CO₂ and B for EtOH(0.05% DEA), Gradient: 10 min @ 40%, Flow rate: 2.0 mL/min, Back pressure: 100 bar, Column temperature: 35° C. |
| 141 | 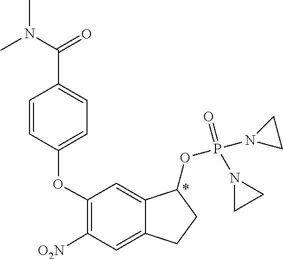 [SMILES: CN(C)C(=O)c1ccc(Oc2cc3c(cc2[N+](=O)[O-])CCC3OP(=O)(N2CC2)N2CC2)cc1] | LCMS(ESI): m/z 473.2[M + H]⁺; ¹H NMR (400 MHz, CDCl₃) δ 7.84 (s, 1H), 7.45-7.42 (m, 2H), 7.26 (s, 1H), 7.04-7.00 (m, 2H), 5.96-5.91 (m, 1H), 3.19- 3.12 (m, 1H), 3.10- 3.03 (m, 6H), 2.96-2.88 (m, 1H), 2.67-2.59 (m, 1H), 2.35-2.27 (m, 1H), 2.20-2.07 (m, 8H). ³¹P NMR (162 MHz, CDCl₃) δ 30.01 (s). ee: >99% Retention time: 3.907 min; Column: ChiralPak IA, 250 × 4.6 mm I.D., 5 μm, Mobile phase: A for CO₂ and B for EtOH(0.05% DEA), Gradient: 10 min @ 40%, Flow rate: 2.0 mL/min, Back pressure: 100 bar, Column temperature: 35° C. |
| 142 | 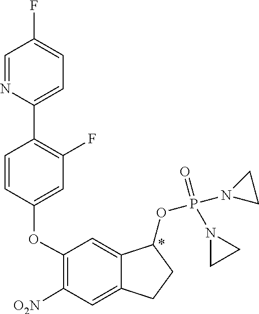 [SMILES: O=[N+]([O-])c1cc2c(cc1Oc1ccc(-c3ccc(F)cn3)c(F)c1)C(OP(=O)(N1CC1)N1CC1)CC2] | LCMS(ESI): m/z 515.1[M + H]⁺; ¹H NMR (400 MHz, CDCl₃) δ 8.55 (s, 1H), 7.96 (t, J = 8.7 Hz, 1H), 7.86 (s, 1H), 7.81-7.75 (m, 1H), 7.47 (t, J = 8.4 Hz, 1H), 7.36 (s, 1H), 6.90 (d, J = 8.6 Hz, 1H), 6.80 (d, J = 12.0 Hz, 1H), 5.99-5.91 (m, 1H), 3.21-3.14 (m, 1H), 2.99-2.89 (m, 1H), 2.68-2.60 (m, 1H), 2.37-2.29 (m, 1H), 2.22-2.08 (m, 8H). ³¹P NMR (162 MHz, CDCl₃) δ 29.97 (s). ¹⁹F NMR (377 MHz, CDCl₃) δ −113.58 (s), −128.60 (s). ee: >99% Retention time: 4.297 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 143 | 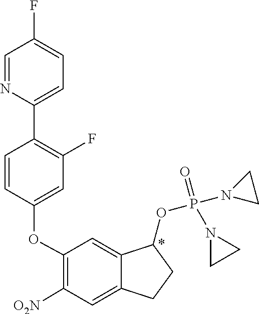 [SMILES: O=[N+]([O-])c1cc2c(cc1Oc1ccc(-c3ccc(F)cn3)c(F)c1)C(OP(=O)(N1CC1)N1CC1)CC2] | LCMS(ESI): m/z 515.1[M + H]⁺; ¹H NMR (400 MHz, CDCl₃) δ 8.55 (s, 1H), 7.97 (t, J = 8.7 Hz, 1H), 7.86 (s, 1H), 7.82-7.72 (m, 1H), 7.47 (t, J = 8.4 Hz, 1H), 7.36 (s, 1H), 6.90 (d, J = 8.6 Hz, 1H), 6.80 (d, J = 12.1 Hz, 1H), 6.01-5.90 (m, 1H), 3.23-3.10 (m, 1H), 3.00-2.88 (m, 1H), 2.72-2.57 (m, 1H), 2.42-2.27 (m, 1H), 2.22-2.09 (m, 8H). ³¹P NMR (162 MHz, CDCl₃) δ 29.96 (s). ¹⁹F NMR (377 MHz, CDCl₃) δ −113.58 (s), −128.62 (s). ee: >99% Retention time: 3.151 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 144 | 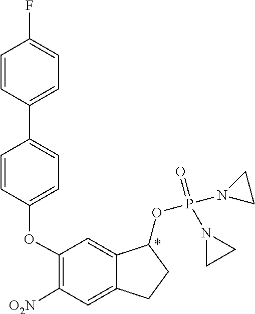 [SMILES: O=[N+]([O-])c1cc2c(cc1Oc1ccc(-c3ccc(F)cc3)cc1)C(OP(=O)(N1CC1)N1CC1)CC2] | LCMS(ESI): m/z 496.1[M + H]⁺; ¹H NMR (400 MHz, CDCl₃) δ 7.82 (s, 1H), 7.59-7.41 (m, 4H), 7.23 (s, 1H), 7.17-7.02 (m, 4H), 6.04-5.82 (m, 1H), 3.21-3.07 (m, 1H), 2.96- 2.84 (m, 1H), 2.69-2.55 (m, 1H), 2.36-2.23 (m, 1H), 2.20-2.00 (m, 8H). ³¹P NMR (162 MHz, CDCl₃) δ 29.95 (s). ¹⁹F NMR (376 MHz, CDCl₃) δ −115.59 (s). ee: 98% Retention time: 2.566 min; Column: ChiralPak IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 145 | 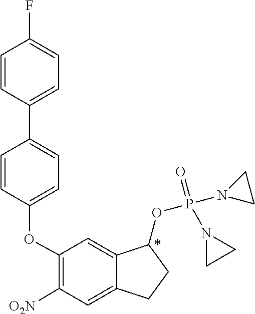 [SMILES: O=[N+]([O-])c1cc2c(cc1Oc1ccc(-c3ccc(F)cc3)cc1)C(OP(=O)(N1CC1)N1CC1)CC2] | LCMS(ESI): m/z 496.1[M + H]⁺; ¹H NMR (400 MHz, CDCl₃) δ 7.82 (s, 1H), 7.60-7.42 (m, 4H), 7.23 (s, 1H), 7.18-6.98 (m, 4H), 6.08-5.76 (m, 1H), 3.22-3.05 (m, 1H), 2.97- 2.83 (m, 1H), 2.69-2.55 (m, 1H), 2.37-2.23 (m, 1H), 2.21-1.97 (m, 8H). ³¹P NMR (162 MHz, CDCl₃) δ 29.95 (s). ¹⁹F NMR (376 MHz, CDCl₃) δ −115.59 (s). ee: 99% Retention time: 2.115 min; Column: ChiralPak IH, 100 × 4.6 mm I.D., 5 μm; Mobile phase: A for CO₂ and B for ethanol (0.05% DEA); Gradient: 8 min @ 30% B; Flow rate: 2.5 mL/min; Column temperature: 40° C. |
| 146 | 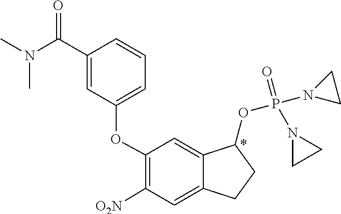 [SMILES: CN(C)C(=O)c1cccc(Oc2cc3c(cc2[N+](=O)[O-])CCC3OP(=O)(N2CC2)N2CC2)c1] | LCMS(ESI): m/z 473.1[M + H]⁺; ¹H NMR (400 MHz, CDCl₃) δ 7.80 (s, 1H), 7.37 (t, J = 7.4 Hz, 1H), 7.23 (s, 1H), 7.16 (d, J = 7.1 Hz, 1H), 7.09-6.98 (m, 2H), 5.97-5.82 (m, 1H), 3.22-3.10 (m, 1H), 3.06 (s, 3H), 2.95 (s, 3H), 2.92-2.78 (m, 1H), 2.70-2.52 (m, 1H), 2.34-2.21 (m, 1H), 2.19-2.03 (m, 8H). ³¹P NMR (162 MHz, CDCl₃) δ 29.91 (s). ee: 99% Retention time: 3.916 min; Column: ChiralPak IH, 100 × 4.6 mm I.D., 5 μm; Mobile phase: A for CO₂ and B for ethanol (0.05% DEA); Gradient: 8 min @ 20% B; Flow rate: 2.5 mL/min; Column temperature: 40° C. |
| 147 | 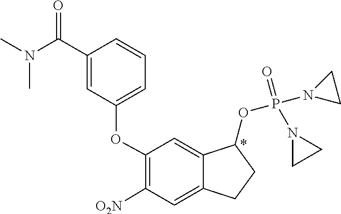 [SMILES: CN(C)C(=O)c1cccc(Oc2cc3c(cc2[N+](=O)[O-])CCC3OP(=O)(N2CC2)N2CC2)c1] | LCMS(ESI): m/z 473.1[M + H] ; ¹H NMR (400 MHz, CDCl₃) δ 7.80 (s, 1H), 7.37 (t, J = 7.2 Hz, 1H), 7.22 (s, 1H), 7.16 (d, J = 7.1 Hz, 1H), 7.08-6.91 (m, 2H), 5.96-5.82 (m, 1H), 3.25-3.11 (m, 1H), 3.05 (s, 3H), 2.95 (s, 3H), 2.91-2.78 (m, 1H), 2.68-2.53 (m, 1H), 2.37-2.24 (m, 1H), 2.18-2.03 (m, 8H). ³¹P NMR (162 MHz, CDCl₃) δ 29.91 (s). ee: >99% Retention time: 3.266 min; Column: ChiralPak IH, 100 × 4.6 mm I.D., 5 μm; Mobile phase: A for CO₂ and B for ethanol (0.05% DEA); Gradient: 8 min @ 20% B; Flow rate: 2.5 mL/min; Column temperature: 40° C. |
| 148 | 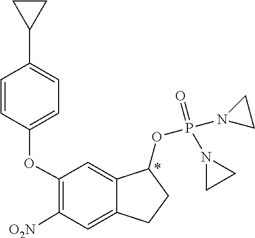 [SMILES: O=[N+]([O-])c1cc2c(cc1Oc1ccc(C3CC3)cc1)C(OP(=O)(N1CC1)N1CC1)CC2] | LCMS(ESI): m/z 442.1[M + H]⁺; ¹H NMR (400 MHz, CDCl₃) δ 7.71 (s, 1H), 7.02- 6.99 (m, 3H), 6.89- 6.86 (m, 2H), 5.84-5.79 (m, 1H), 3.08-3.00 (m, 1H), 2.84-2.76 (m, 1H), 2.56-2.48 (m, 1H), 2.23-2.16 (m, 1H), 2.10-2.08 (m, 1H), 2.06-2.04 (m, 1H), 2.02-1.93 (m, 6H), 1.86-1.79 (m, 1H), 0.92-0.87 (m, 2H), 0.60- 0.56 (m, 2H). ³¹P NMR (162 MHz, CDCl₃) δ 29.91 (s). ee: 97% Retention time: 3.295 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 149 | 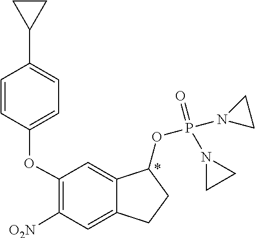 [SMILES: O=[N+]([O-])c1cc2c(cc1Oc1ccc(C3CC3)cc1)C(OP(=O)(N1CC1)N1CC1)CC2] | LCMS(EST): m/z 442.1[M + H]⁺; ¹H NMR (400 MHz, CDCl₃) δ 7.71 (s, 1H), 7.01-6.99 (m, 3H), 6.88- 6.56 (m, 2H), 5.84-5.79 (m, 1H), 3.08-3.00 (m, 1H), 2.84-2.76 (m, 1H), 2.56-2.48 (m, 1H), 2.23-2.15 (m, 1H), 2.10-2.08 (m, 1H), 2.07-2.05 (m, 1H), 2.02-1.93 (m, 6H), 1.86-1.79 (m, 1H), 0.92-0.87 (m, 2H), 0.60- 0.56 (m, 2H). ³¹P NMR (162 MHz, CDCl₃) δ 29.91 (s). ee: >99% Retention time: 2.866 min; Column: ChiralPak C-IG, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 150 | 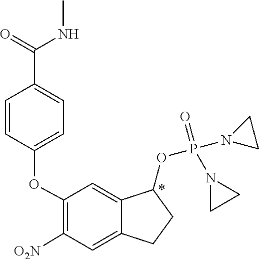 [SMILES: CNC(=O)c1ccc(Oc2cc3c(cc2[N+](=O)[O-])CCC3OP(=O)(N2CC2)N2CC2)cc1] | LCMS(ESI): m/z 459.1[M + H]⁺; ¹H NMR (400 MHz, CDCl₃) δ 7.78 (s, 1H), 7.72-7.66 (m, 2H), 7.21 (s, 1H), 6.98-6.91 (m, 2H), 6.14-6.04 (m, 1H), 5.89-5.84 (m, 1H), 3.15- 3.04 (m, 1H), 2.94 (d, J = 4.8 Hz, 3H), 2.89-2.83 (m, 1H), 2.59-2.54 (m, 1H), 2.29-2.21 (m, 1H), 2.15-2.11 (m, 1H), 2.10-1.98 (m, 7H). ³¹P NMR (162 MHz, CDCl₃) δ 30.00 (s). ee: 99% Retention time: 3.897 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 151 | 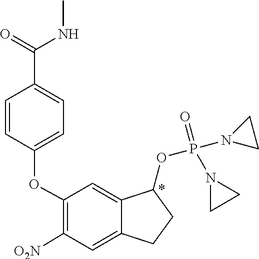 [SMILES: CNC(=O)c1ccc(Oc2cc3c(cc2[N+](=O)[O-])CCC3OP(=O)(N2CC2)N2CC2)cc1] | LCMS(ESI): m/z 459.1[M + H]⁺; ¹H NMR (400 MHz, CDCl₃) δ 7.78 (s, 1H), 7.71-7.67 (m, 2H), 7.21 (s, 1H), 6.97-6.93 (m, 2H), 6.13-6.03 (m, 1H), 5.89-5.84 (m, 1H), 3.13- 3.06 (m, 1H), 2.94 (d, J = 4.8 Hz, 3H), 2.89-2.82 (m, 1H), 2.61-2.52 (m, 1H), 2.28-2.20 (m, 1H), 2.14-2.12 (m, 1H), 2.10-1.97 (m, 7H). ³¹P NMR (162 MHz, CDCl₃) δ 29.98 (s). ee: >99% Retention time: 3.275 min; Column: ChiralPak AD, 250 × 4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. |
| 152 | 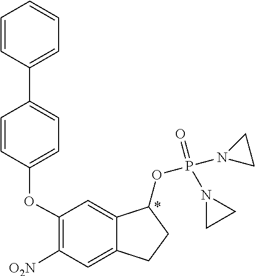 [SMILES: O=[N+]([O-])c1cc2c(cc1Oc1ccc(-c3ccccc3)cc1)C(OP(=O)(N1CC1)N1CC1)CC2] | LCMS(ESI): m/z 478.2[M + H]⁺; ¹H NMR (400 MHz, CDCl₃) δ 7.82 (s, 1H), 7.59-7.54 (m, 4H), 7.46- 7.42 (m, 2H), 7.37-7.33 (m, 1H), 7.23 (s, 1H), 7.12-7.10 (m, 2H), 5.95- 5.90 (m, 1H), 3.18-3.10 (m, 1H), 2.94-2.86 (m, 1H), 2.66-2.57 (m, 1H), 2.32-2.24 (m, 1H), 2.17-2.15 (m, 1H), 2.13-2.00 (m, 7H). ³¹P NMR (162 MHz, CDCl₃) δ 29.98 (s). ee: 98% Retention time: 4.126 min; Column: ChiralCel OJ, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 153 | 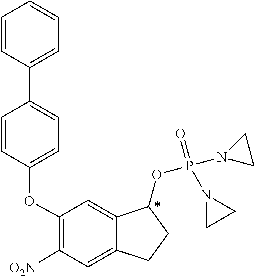 [SMILES: O=[N+]([O-])c1cc2c(cc1Oc1ccc(-c3ccccc3)cc1)C(OP(=O)(N1CC1)N1CC1)CC2] | LCMS(ESI): m/z 478.2[M + H]⁺; ¹H NMR (400 MHz, CDCl₃) 6 7.82 (s, 1H), 7.60-7.54 (m, 4H), 7.46- 7.42 (m, 2H), 7.37-7.33 (m, 1H), 7.23 (s, 1H), 7.13-7.08 (m, 2H), 5.95- 5.90 (m, 1H), 3.18-3.10 (m, 1H), 2.94-2.86 (m, 1H), 2.66-2.57 (m, 1H), 2.33-2.24 (m, 1H), 2.17-2.16 (m, 1H), 2.14-2.01 (m, 7H). ³¹P NMR (162 MHz, CDCl₃) δ 29.98 (s). ee: 95% Retention time: 3.708 min; Column: ChiralCel OJ, 250 × 4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C. |
| 154 | 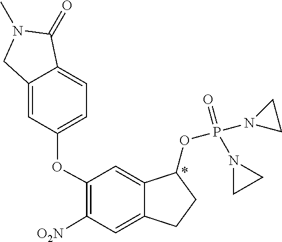 [SMILES: CN1Cc2cc(Oc3cc4c(cc3[N+](=O)[O-])CCC4OP(=O)(N3CC3)N3CC3)ccc2C1=O] | LCMS(ESI): m/z 471.1[M + H]⁺; ¹H NMR (400 MHz, CDCl₃) δ 7.84 (s, 1H), 7.79-7.77 (m, 1H), 7.28 (s, 1H), 7.04-7.02 (m, 2H), 5.95-5.90 (m, 1H), 4.34 (s, 2H), 3.19-3.12 (m, 4H), 2.96-2.90 (m, 1H), 2.65-2.60 (m, 1H), 2.34-2.26 (m, 1H), 2.19- 2.18(m, 1H), 2.16-2.03 (m, 7H). ³¹P NMR (162 MHz, CDCl₃) δ 30.02 (s). ee: 99% Retention time: 5.201 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 155 | 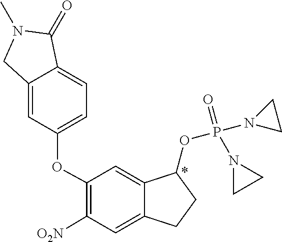 [SMILES: CN1Cc2cc(Oc3cc4c(cc3[N+](=O)[O-])CCC4OP(=O)(N3CC3)N3CC3)ccc2C1=O] | LCMS(ESI): m/z 471.1[M + H]⁺; ¹H NMR (400 MHz, CDCl₃) δ 7.84 (s, 1H), 7.79-7.77 (m, 1H), 7.28 (s, 1H), 7.04-7.02 (m, 2H), 5.95-5.90 (m, 1H), 4.34 (s, 2H), 3.19-3.12 (m, 4H), 2.96-2.88 (m, 1H), 2.67-2.59 (m, 1H), 2.34-2.26 (m, 1H), 2.20- 2.18 (m, 1H), 2.17-2.03 (m, 7H). ³¹P NMR (162 MHz, CDCl₃) δ 30.02 (s). ee: >99% Retention time: 3.968 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 156 | 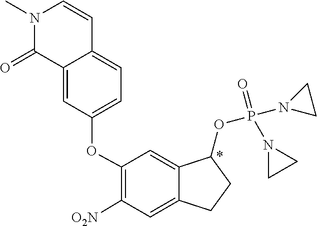 [SMILES: Cn1ccc2ccc(Oc3cc4c(cc3[N+](=O)[O-])CCC4OP(=O)(N3CC3)N3CC3)cc2c1=O] | LCMS(ESI): m/z 483.1[M + H]⁺; ¹H NMR (400 MHz, CDCl₃) δ 7.85-7.72 (m, 2H), 7.52-7.43 (m, 1H), 7.39-7.30 (m, 1H), 7.22 (s, 1H), 7.03- 6.84 (m, 1H), 6.50-6.31 (m, 1H), 5.92-5.77 (m, 1H), 3.50 (s, 3H), 3.14-3.03 (m, 1H), 2.91-2.76 (m, 1H), 2.65- 2.45 (m, 1H), 2.30-2.17 (m, 1H), 2.15-1.92 (m, 8H). ³¹P NMR (162 MHz, CDCl₃) δ 29.65 (s). ee: 99% Retention time: 2.336 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 157 | 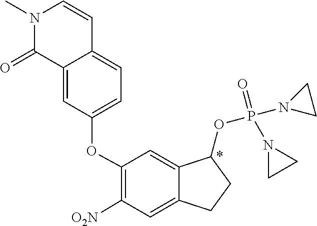 [SMILES: Cn1ccc2ccc(Oc3cc4c(cc3[N+](=O)[O-])CCC4OP(=O)(N3CC3)N3CC3)cc2c1=O] | LCMS(ESI): m/z 483.2[M + H]⁺; ¹H NMR (400 MHz, CDCl₃) δ 7.84-7.69 (m, 2H), 7.48 (d, J = 8.6 Hz, 1H), 7.34 (dd, J = 8.6, 2.6 Hz, 1H), 7.22 (s, 1H), 6.96 (d, J = 7.3 Hz, 1H), 6.42 (d, J = 7.3 Hz, 1H), 5.88-5.79 (m, 1H), 3.49 (d, J = 9.8 Hz, 3H), 3.14-3.06 (m, 1H), 2.93-2.77 (m, 1H), 2.64-2.47 (m, 1H), 2.32- 2.17 (m, 1H), 2.15-1.90 (m, 8H). ³¹P NMR (162 MHz, CDCl₃) δ 29.65 (s). ee: >99% Retention time: 1.816 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 158 | 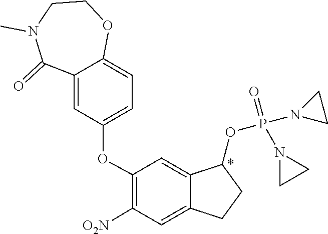 [SMILES: CN1CCOc2ccc(Oc3cc4c(cc3[N+](=O)[O-])CCC4OP(=O)(N3CC3)N3CC3)cc2C1=O] | LCMS(ESI): m/z 501.2 [M + H]⁺; ¹H NMR (400 MHz, CDCl₃) δ 7.80 (s, 1H), 7.39 (d, J = 3.0 Hz, 1H), 7.22 (s, 1H), 7.14-7.10 (m, 1H), 7.03-6.99 (m, 1H), 5.93-5.86 (m, 1H), 4.38 (t, J = 4.0 Hz, 2H), 3.53 (t, J = 4.0 Hz, 2H), 3.19 (s, 3H), 3.14-3.11 (m, 1H), 2.92-2.86 (m, 1H), 2.64-2.55 (m, 1H), 2.32-2.25 (m, 1H), 2.20-2.05 (m, 8H). ³¹P NMR (162 MHz, CDCl₃) δ 29.70 (s). ee: 97% Retention time: 4.299 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 159 | 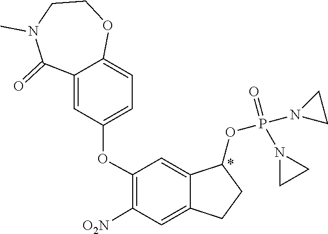 [SMILES: CN1CCOc2ccc(Oc3cc4c(cc3[N+](=O)[O-])CCC4OP(=O)(N3CC3)N3CC3)cc2C1=O] | LCMS(ESI): m/z 501.2 [M + H]⁺; ¹H NMR (400 MHz, CDCl₃) δ 7.80 (s, 1H), 7.39 (d, J = 3.0 Hz, 1H), 7.22 (s, 1H), 7.13-7.10 (m, 1H), 7.03-6.99 (d, J = 8.8 Hz, 1H), 5.94- 5.85 (m, 1H), 4.38 (t, J = 4.0 Hz, 2H), 3.53 (t, J = 4.0 Hz, 2H), 3.19 (s, 3H), 3.12-3.10 (m, 1H), 2.92-2.88 (m, 1H), 2.65-2.56 (m, 1H), 2.33- 2.26 (m, 1H), 2.18-2.06 (m, 8H). ³¹P NMR (162 MHz, CDCl₃) δ 29.71 (s). ee: >99% Retention time: 3.402 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 160 | 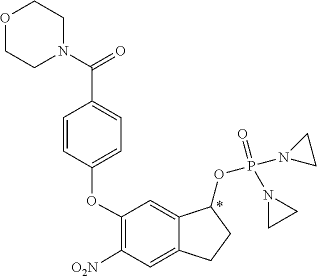 [SMILES: O=C(c1ccc(Oc2cc3c(cc2[N+](=O)[O-])CCC3OP(=O)(N2CC2)N2CC2)cc1)N1CCOCC1] | LCMS(ESI): m/z 515.2 [M + H]⁺; ¹H NMR (400 MHz, CDCl₃) δ 7.83(s, 1H), 7.42-7.40 (m, 2H), 7.28 (s, 1H), 7.02-6.99 (m, 2H), 5.94-5.90 (m, 1H), 3.79-3 .. 52 (m, 8H), 3.15- 3.11 (m, 1H), 2.93-2.89 (m, 1H), 2.63-2.60 (m, 1H), 2.34-2.26 (m, 1H), 2.20-2.07 (m, 8H). ³¹P NMR (162 MHz, CDCl₃) δ 29.99 (s). ee: 99% Retention time: 5.926 min; Column: ChiralPak AD, 250 × 4.6 mm I.D., 5 μm, Mobile phase: A for CO₂ and B for methanol, Gradient: 8 min @B 40%, Flow rate: 2.0 mL/min, Back pressure: 100 bar, Column temperature: 35° C. |
| 161 | 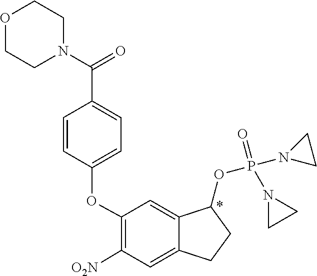 [SMILES: O=C(c1ccc(Oc2cc3c(cc2[N+](=O)[O-])CCC3OP(=O)(N2CC2)N2CC2)cc1)N1CCOCC1] | LCMS(ESI): m/z 515.2 [M + H]⁺; ¹H NMR (400 MHz, CDCl₃) δ 7.83 (s, 1H), 7.42-7.39 (m, 2H), 7.27- 7.26 (m, 1H), 7.01-6.99 (m, 2H), 5.94-5.89 (m, 1H), 3.83-3.51 (s, 8H), 3.14-3.11 (m, 1H), 2.93-2.89 (m, 1H), 2.66-2.58 (m, 1H), 2.34-2.25 (m, 1H), 2.20-2.07 (m, 8H). ³¹P NMR (162 MHz, CDCl₃) δ 29.97 (s). ee: 96% Retention time: 4.287 min; Column: ChiralPak AD, 250 × 4.6 mm I.D., 5 μm, Mobile phase: A for CO₂ and B for methanol, Gradient: 8 min @B 40%, Flow rate: 2.0 mL/min, Back pressure: 100 bar, Column temperature: 35° C. |
| 162 | 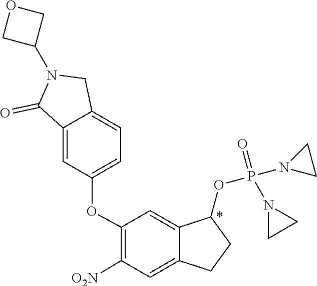 [SMILES: O=C1c2cc(Oc3cc4c(cc3[N+](=O)[O-])CCC4OP(=O)(N3CC3)N3CC3)ccc2CN1C1COC1] | LCMS(ESI): m/z 513.2 [M + H]⁺; ¹H NMR (400 MHz, CDCl₃) δ 7.86 (s, 1H), 7.54-7.50 (m, 1H), 7.35- 7.30 (m, 2H), 7.27-7.25 (m, 1H), 5.95-5.87 (m, 1H), 5.67-5.57 (m, 1H), 4.99 (t, J = 7.4 Hz, 2H), 4.87 (t, J = 6.7 Hz, 2H), 4.69 (s, 2H), 3.24-3.14 (m, 1H), 2.99-2.89 (m, 1H), 2.69-2.57 (m, 1H), 2.39-2.27 (m, 1H), 2.20-2.08 (m, 8H). ³¹P NMR (162 MHz, CDCl₃) δ 29.71 (s). ee: 99% Retention time: 2.253 min; Column: ChiralPak IH, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 163 | 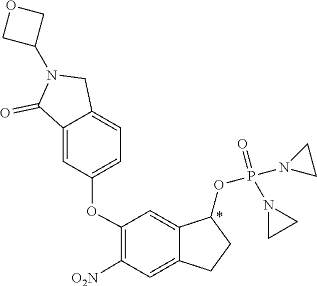 [SMILES: O=C1c2cc(Oc3cc4c(cc3[N+](=O)[O-])CCC4OP(=O)(N3CC3)N3CC3)ccc2CN1C1COC1] | LCMS(ESI): m/z 513.1 [M + H]⁺; ¹H NMR (400 MHz, CDCl₃) δ 7.86 (s, 1H), 7.55-7.50 (m, 1H), 7.35- 7.31 (m, 2H), 7.29-7.27 (m, 1H), 5.96-5.89 (m, 1H), 5.67-5.57 (m, 1H), 4.99 (t, J = 7.3 Hz, 2H), 4.87 (t, J = 6.7 Hz, 2H), 4.69 (s, 2H), 3.24-3.13 (m, 1H), 2.99-2.88 (m, 1H), 2.68-2.57 (m, 1H), 2.38-2.28 (m, 1H), 2.24-2.06 (m, 8H). ³¹P NMR (162 MHz, CDCl₃) δ 29.71 (s). ee: >99% Retention time: 1.681 min; Column: ChiralPak IH, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 164 | 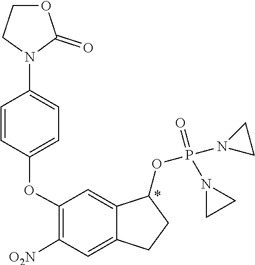 [SMILES: O=C1OCCN1c1ccc(Oc2cc3c(cc2[N+](=O)[O-])CCC3OP(=O)(N2CC2)N2CC2)cc1] | LCMS(ESI): m/z 487.2 [M + H]⁺; ¹H NMR (400 MHz, CDCl₃) δ 7.80 (s, 1H), 7.59-7.49 (m, 2H), 7.16 (s, 1H), 7.09-7.03 (m, 2H), 5.94-5.85 (m, 1H), 4.56-4.45 (m, 2H), 4.13- 4.00 (m, 2H), 3.19-3.06 (m, 1H), 2.95-2.81 (m, 1H), 2.66-2.53 (m, 1H), 2.36- 2.23 (m, 1H), 2.21-2.00 (m, 8H). ³¹P NMR (162 MHz, CDCl₃) δ 29.87 (s). ee: 94% Retention time: 3.099 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 165 | 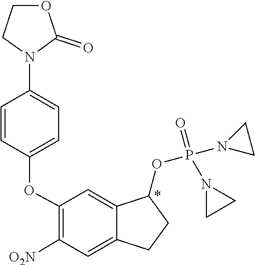 [SMILES: O=C1OCCN1c1ccc(Oc2cc3c(cc2[N+](=O)[O-])CCC3OP(=O)(N2CC2)N2CC2)cc1] | LCMS(ESI): m/z 487.1 [M + H]⁺; ¹H NMR (400 MHz, CDCl₃) δ 7.80 (s, 1H), 7.54 (d, J = 8.5 Hz, 2H), 7.16 (s, 1H), 7.06 (d, J = 8.4 Hz, 2H), 5.94-5.83 (m, 1H), 4.50 (t, J = 7.9 Hz, 2H), 4.07 (t, J = 7.9 Hz, 2H), 3.19-3.09 (m, 1H), 2.94-2.84 (m, 1H), 2.65-2.55 (m, 1H), 2.34-2.23 (m, 1H), 2.19-2.04 (m, 8H). ³¹P NMR (162 MHz, CDCl₃) δ 29.89 (s). ee: >99% Retention time: 2.635 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 166 | 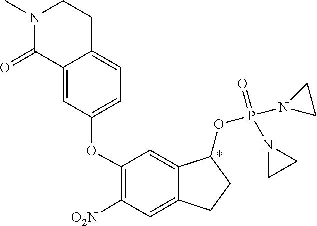 [SMILES: CN1CCc2ccc(Oc3cc4c(cc3[N+](=O)[O-])CCC4OP(=O)(N3CC3)N3CC3)cc2C1=O] | LCMS(ESI): m/z 485.1 [M + H]⁺; ¹H NMR (400 MHz, CDCl₃) δ 7.78 (s, 1H), 7.59 (d, J = 2.5 Hz, 1H), 7.16 (d, J = 8.5 Hz, 2H), 7.09 (dd, J = 8.2, 2.6 Hz, 1H), 5.89-5.81 (m, 1H), 3.53 (t, J = 6.7 Hz, 2H), 3.15-3.08 (m, 4H), 2.96 (t, J = 6.7 Hz, 2H), 2.91-2.82 (m, 1H), 2.61-2.51 (m, 1H), 2.30-2.22 (m, 1H), 2.15- 2.02 (m, 8H). ³¹P NMR (162 MHz, CDCl₃) δ 29.60 (s). ee: 98% Retention time: 4.128 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 167 | 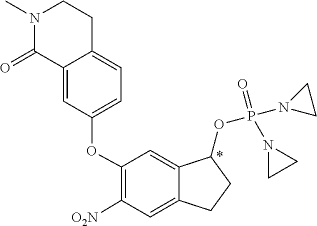 [SMILES: CN1CCc2ccc(Oc3cc4c(cc3[N+](=O)[O-])CCC4OP(=O)(N3CC3)N3CC3)cc2C1=O] | LCMS(ESI): m/z 485.1 [M + H]⁺; ¹H NMR (400 MHz, CDCl₃) δ 7.78 (s, 1H), 7.59 (d, J = 2.5 Hz, 1H), 7.15 (d, J = 8.0 Hz, 2H), 7.09 (dd, J = 8.2, 2.6 Hz, 1H), 5.89-5.82 (m, 1H), 3.53 (t, J = 6.7 Hz, 2H), 3.11-3.07 (m, 4H), 2.95 (t, J = 6.7 Hz, 2H), 2.91-2.82 (m, 1H), 2.61-2.51 (m, 1H), 2.29-2.23 (m, 1H), 2.17-2.01 (m, 8H) ³¹P NMR (162 MHz, CDCl₃) δ 29.61 (s) ee: >99% Retention time: 3.176 min; Column: ChiralPak IH, 100 × 4.6 mm I.D., 5 μm; Mobile phase: A for CO₂ and B for methanol (0.05% DEA); Gradient: 8 min @ 20% B; Flow rate: 2.5 mL/min; Column temperature: 40° C. |
| 168 | 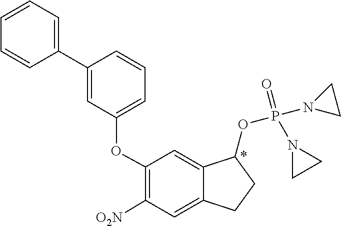 [SMILES: O=[N+]([O-])c1cc2c(cc1Oc1cccc(-c3ccccc3)c1)C(OP(=O)(N1CC1)N1CC1)CC2] | LCMS(ESI): m/z 478.1 [M + H]⁺; ¹H NMR (400 MHz, CDCl₃) δ 7.81 (s, 1H), 7.55 (d, J = 7.4 Hz, 2H), 7.46-7.39 (m, 4H), 7.37-7.31 (m, 1H), 7.27 (s, 1H), 7.22 (s, 1H), 7.05- 7.00 (m, 1H), 5.94-5.85 (m, 1H), 3.18-3.08 (m, 1H), 2.94-2.83 (m, 1H), 2.66-2.54 (m, 1H), 2.32-2.23 (m, 1H), 2.13-1.96 (m, 8H). ³¹P NMR (162 MHz, CDCl₃) δ 29.86 (s) ee: 96% Retention time: 1.950 min; Column: ChiralPak IH, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 169 | 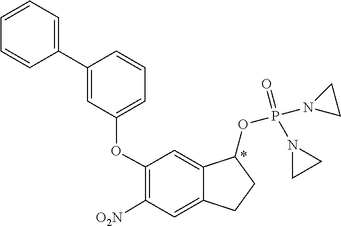 [SMILES: O=[N+]([O-])c1cc2c(cc1Oc1cccc(-c3ccccc3)c1)C(OP(=O)(N1CC1)N1CC1)CC2] | LCMS(ESI): m/z 478.1 [M + H]⁺; ¹H NMR (400 MHz, CDCl₃) δ 7.78 (s, 1H), 7.52 (d, J = 7.4 Hz, 2H), 7.44-7.36 (m, 4H), 7.34-7.28 (m, 1H), 7.25 (s, 1H), 7.19 (s, 1H), 7.02- 6.97 (m, 1H), 5.91- 5.84 (m, 1H), 3.15-3.05 (m, 1H), 2.92-2.81 (m, 1H), 2.62-2.52 (m, 1H), 2.29-2.20 (m, 1H), 2.10-1.91 (m, 8H). ³¹P NMR (162 MHz, CDCl₃) δ 29.86 (s). ee: >99% Retention time: 1.618 min; Column: ChiralPak IH, 100 × 4.6 mm I.D., 5 μm; Mobile phase: A for CO₂ and B for methanol (0.05% DEA); Gradient: 8 min @ 40% B; Flow rate: 2.5 mL/min; Column temperature: 40° C. |
| 170 | 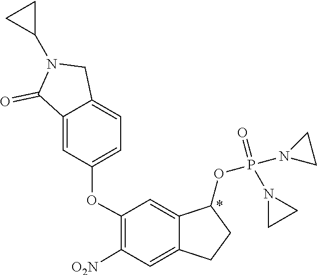 [SMILES: O=C1c2cc(Oc3cc4c(cc3[N+](=O)[O-])CCC4OP(=O)(N3CC3)N3CC3)ccc2CN1C1CC1] | LCMS(ESI): m/z 497.1 [M + H]⁺; ¹H NMR (400 MHz, CDCl₃) δ 7.85 (s, 1H), 7.43-7.38 (m, 1H), 7.28- 7.27 (m, 3H), 5.93-5.88 (m, 1H), 4.31 (s, 2H), 3.26-3.10 (m, 1H), 3.02- 2.86 (m, 2H), 2.63-2.61 (m, 1H), 2.39-2.27 (m, 1H), 2.25-2.02 (m, 8H), 1.01-0.79 (m, 4H) ³¹P NMR (162 MHz, CDCl₃) δ 29.69 (s). ee: 98% Retention time: 4.425 min; Column: ChiralPak IH, 100 × 4.6 mm I.D., 5 μm; Mobile phase: A for CO₂ and B for methanol (0.05% DEA); Gradient: 0.0 min-1.0 min @ 10% B, 1.0 min-4.5min gradient (10-40% B), 4.5min-7.0 min @ 40% B, 7.0 min-8.0 min @ 10% B; Flow rate: 2.5 mL/min; Column temperature: 40° C. |
| 171 | 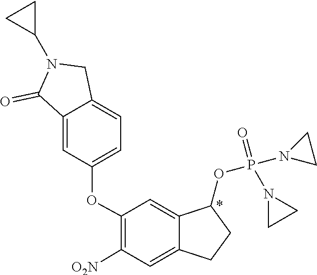 [SMILES: O=C1c2cc(Oc3cc4c(cc3[N+](=O)[O-])CCC4OP(=O)(N3CC3)N3CC3)ccc2CN1C1CC1] | LCMS(ESI): m/z 497.1 [M + H]⁺; ¹H NMR (400 MHz, CDCl₃) δ 8 7.85 (s, 1H), 7.43-7.38 (m, 1H)., 7.28- 7.27 (m, 3H), 5.93-5.88 (m, 1H), 4.31 (s, 2H), 3.26-3.10 (m, 1H), 3.02- 2.86 (m, 2H), 2.63-2.61 (m, 1H), 2.39-2.27 (m, 1H), 2.25-2.02 (m, 8H), 1.01-0.79 (m, 4H) ³¹P NMR (162 MHz, CDCl₃) δ 29.69 (s). ee: >99% Retention time: 4.029 min; Column: ChiralPak IH, 100 × 4.6 mm I.D., 5 μm; Mobile phase: A for CO₂ and B for methanol (0.05% DEA); Gradient: 0.0 min-1.0 min @ 10% B, 1.0 min-4.5min gradient (10-40% B), 4.5min-7.0 min @ 40% B, 7.0 min-8.0 min @ 10% B; Flow rate: 2.5 mL/min; Column temperature: 40° C. |
| 172 | 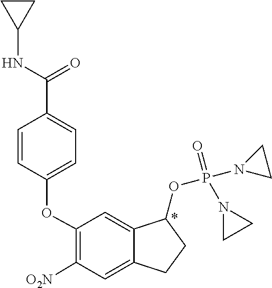 [SMILES: O=C(NC1CC1)c1ccc(Oc2cc3c(cc2[N+](=O)[O-])CCC3OP(=O)(N2CC2)N2CC2)cc1] | LC-MS (ESI): m/z 485.1 [M + H]⁺; ¹H NMR (400 MHz, CDCl₃) δ 7.84 (s, 1H), 7.74 (d, J = 8.8 Hz, 2H), 7.27 (s, 1H), 7.03-6.97 (m, 2H), 6.27 (s, 1H), 5.97-5.89 (m, 1H), 3.22-3.11 (m, 1H), 2.98-2.85 (m, 2H), 2.70-2.57 (m, 1H), 2.38-2.25 (m, 1H), 2.23-2.05 (m, 8H), 0.91-0.84 (m, 2H), 0.65-0.58 (m, 2H). ³¹P NMR (162 MHz, CDCl₃) δ 29.97 (s). ee: 97% Retention time: 3.370 min; Column: ChiralPak C-IG, 250 × 30 mm I.D., 5 μm, Mobile phase: A for CO₂ and B for MEOH + 0.1%NH₃H₂O, Gradient: B 40%; Flow rate: 70 mL/min, Back pressure: 100 bar, Column temperature: 35° C. |
| 173 | 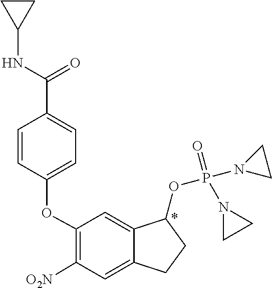 [SMILES: O=C(NC1CC1)c1ccc(Oc2cc3c(cc2[N+](=O)[O-])CCC3OP(=O)(N2CC2)N2CC2)cc1] | LC-MS (ESI): m/z 485.1 [M + H]⁺; ¹H NMR (400 MHz, CDCl₃) δ 7.84 (s, 1H), 7.73 (d, J = 8.7 Hz, 2H), 7.26(s, 1H), 7.00 (t, J = 8.0 Hz, 2H), 6.31 (s, 1H), 5.96-5.91 (m, 1H), 3.24-3.11 (m, 1H), 2.99-2.86 (m, 2H), 2.71-2.57 (m, 1H), 2.34- 2.30 (m, 1H), 2.25-2.04 (m, 8H), 0.89-0.84 (m, 2H), 0.69-0.57 (m, 2H). ³¹P NMR (162 MHz, CDCl₃) δ 29.97 (s). ee: 97% Retention time: 4.064 min; Column: ChiralPak C-IG, 250 × 30 mm I.D., 5 μm, Mobile phase: A for CO₂ and B for MEOH + 0.1%NH₃H₂O, Gradient: B 40%; Flow rate: 70 mL/min, Back pressure: 100 bar, Column temperature: 35° C. |
| 174 | 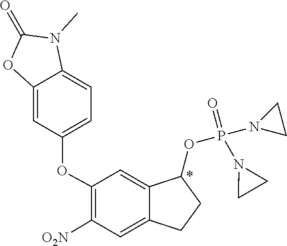 [SMILES: Cn1c(=O)oc2cc(Oc3cc4c(cc3[N+](=O)[O-])CCC4OP(=O)(N3CC3)N3CC3)ccc21] | LC-MS (ESI): m/z 473.1 [M + H]⁺; ¹H NMR (400 MHz, CDCl₃) δ 7.81 (s, 1H), 7.20 (s, 1H), 6.96-6.89 (m, 3H), 5.93-5.85 (m, 1H), 3.42 (s, 3H), 3.21-3.11 (m, 1H), 2.97-2.85 (m, 1H), 2.66-2.54 (m, 1H), 2.35-2.24 (m, 1H), 2.22-2.03 (m, 8H). ³¹P NMR (162 MHz, CDCl₃) δ 29.84 (s). ee: >99% Retention time: 2.528 min |
| 175 | 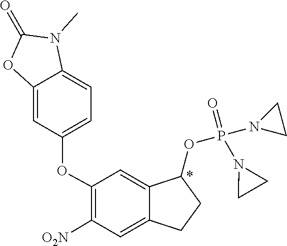 [SMILES: Cn1c(=O)oc2cc(Oc3cc4c(cc3[N+](=O)[O-])CCC4OP(=O)(N3CC3)N3CC3)ccc21] | LC-MS (ESI): m/z 495.1 [M + H]⁺; ¹H NMR (400 MHz, CDCl₃) δ 7.81 (s, 1H), 7.20 (s, 1H), 6.97-6.88 (m, 3H), 5.94-5.86 (m, 1H), 3.42 (s, 3H), 3.21-3.11 (m, 1H), 2.96-2.86 (m, 1H), 2.67-2.55 (m, 1H), 2.37-2.25 (m, 1H), 2.22-2.05 (m, 8H). ³¹P NMR (162 MHz, CDCl₃) δ 29.84 (s). ee: >99% Retention time: 2.046 min |
| 176 | 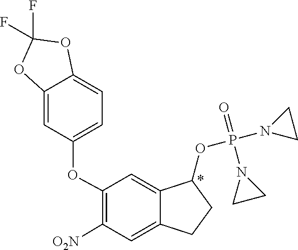 [SMILES: O=[N+]([O-])c1cc2c(cc1Oc1ccc3c(c1)OC(F)(F)O3)C(OP(=O)(N1CC1)N1CC1)CC2] | LC-MS (ESI): m/z 478 [M + H]⁺; ¹H NMR (400 MHz, CDCl₃) δ 7.82 (s, 1H), 7.21 (s, 1H), 7.03 (d, J = 8.7 Hz, 1H), 6.81 (d, J = 2.4 Hz, 1H), 6.74 (dd, J = 8.7, 2.5 Hz, 1H), 5.98-5.85 (m, 1H), 3.21-3.09 (m, 1H), 2.97-2.84 (m, 1H), 2.68-2.55 (m, 1H), 2.37-2.26 (m, 1H), 2.26-1.95 (m, 8H). ³¹P NMR (162 MHz, CDCl₃) δ 29.96 (s). ¹⁹F NMR (376 MHz, CDCl₃) δ −49.12-−50.50 (m). ee: >95% Retention time: 5.446 min |
| 177 | 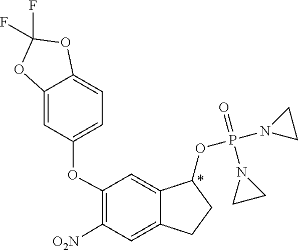 [SMILES: O=[N+]([O-])c1cc2c(cc1Oc1ccc3c(c1)OC(F)(F)O3)C(OP(=O)(N1CC1)N1CC1)CC2] | LC-MS (ESI): m/z 478 [M + H]⁺; ¹H NMR (400 MHz, CDCl₃) δ 7.82 (s, 1H), 7.21 (s, 1H), 7.03 (d, J = 8.7 Hz, 1H), 6.81 (d, J = 2.4 Hz, 1H), 6.74 (dd, J = 8.7, 2.5 Hz, 1H), 5.99-5.86 (m, 1H), 3.20-3.10 (m, 1H), 2.96-2.86 (m, 1H), 2.67-2.56 (m, 1H), 2.36-2.25 (m, 1H), 2.23-2.02 (m, 8H). ³¹P NMR (162 MHz, CDCl₃) δ 29.96 (s). ¹⁹F NMR (376 MHz, CDCl₃) δ −49.38-−50.14 (m). ee: 88% Retention time: 4.885 min |
| 178 | 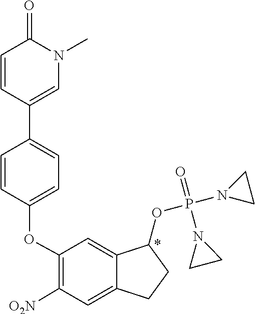 [SMILES: Cn1cc(-c2ccc(Oc3cc4c(cc3[N+](=O)[O-])CCC4OP(=O)(N3CC3)N3CC3)cc2)ccc1=O] | LC-MS (ESI): m/z 509.1 [M + H]⁺; ¹H NMR (400 MHz, CDCl₃) δ 7.80 (s, 1H), 7.56 (dd, J = 9.4, 2.7 Hz, 1H), 7.46 (d, J = 2.5 Hz, 1H), 7.39-7.34 (m, 2H), 7.21 (s, 1H), 7.07- 7.01 (m, 2H), 6.64 (d, J = 9.4 Hz, 1H), 5.93-5.87(m, 1H), 3.61 (s, 3H), 3.18-3.08 (m, 1H), 2.94-2.85 (m, 1H), 2.65-2.55 (m, 1H), 2.34-2.24 (m, 1H), 2.18-2.03 (m, 8H). ³¹P NMR (162 MHz, CDCl₃) δ 29.94 (s). ee: >99% Retention time: 10.257 min |
| 179 | 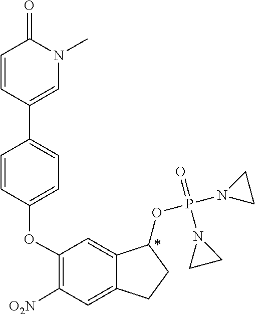 [SMILES: Cn1cc(-c2ccc(Oc3cc4c(cc3[N+](=O)[O-])CCC4OP(=O)(N3CC3)N3CC3)cc2)ccc1=O] | LC-MS (ESI): m/z 509.1 [M + H]⁺; ¹H NMR (400 MHz, CDCl₃) δ 7.81 (s, 1H), 7.57 (dd, J = 9.4, 2.7 Hz, 1H), 7.47 (d, J = 2.6 Hz, 1H), 7.40-7.34 (m, 2H), 7.22 (s, 1H), 7.07- 7.02 (m, 2H), 6.66 (d, J = 9.4 Hz, 1H), 5.94-5.87 (m, 1H), 3.62 (s, 3H), 3.19-3.09 (m, 1H), 2.96-2.84 (m, 1H), 2.66-2.56 (m, 1H), 2.35-2.24 (m, 1H), 2.19-2.03 (m, 8H). ee: >99% Retention time: 7.815 min |
| 180 | 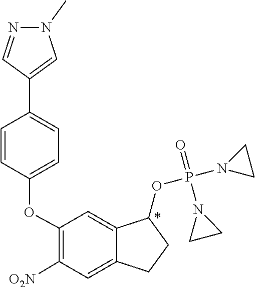 [SMILES: Cn1cc(-c2ccc(Oc3cc4c(cc3[N+](=O)[O-])CCC4OP(=O)(N3CC3)N3CC3)cc2)cn1] | LC-MS (ESI): m/z 482.2 [M + H]⁺; ¹H NMR (400 MHz, CDCl₃) δ 7.80 (s, 1H), 7.71 (s, 1H), 7.58 (s, 1H), 7.45 (d, J = 8.7 Hz, 2H), 7.18 (s, 1H), 7.04 (d, J = 8.7 Hz, 2H), 5.93- 5.86 (m, 1H), 3.95 (s, 3H), 3.17-3.07 (m, 1H), 2.93-2.84 (m, 1H), 2.65- 2.54 (m, 1H), 2.33-2.22 (m, 1H), 2.16-2.01 (m, 8H). ³¹P NMR (162 MHz, CDCl₃) δ 29.97 (s). ee: >99% Retention time: 10.086 min |
| 181 | 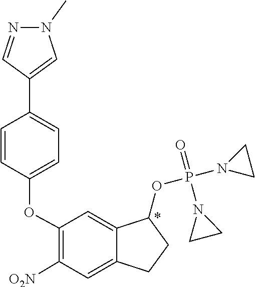 [SMILES: Cn1cc(-c2ccc(Oc3cc4c(cc3[N+](=O)[O-])CCC4OP(=O)(N3CC3)N3CC3)cc2)cn1] | LC-MS (ESI): m/z 482.2 [M + H]⁺; ¹H NMR (400 MHz, CDCl₃) δ 7.80 (s, 1H), 7.71 (s, 1H), 7.58 (s, 1H), 7.47-7.43 (m, 2H), 7.18 (s, 1H), 7.06-7.02 (m, 2H), 5.94-5.87 (m, 1H), 3.95 (s, 3H), 3.17-3.08 (m, 1H), 2.94-2.84 (m, 1H), 2.66-2.56 (m, 1H), 2.33- 2.22 (m, 1H), 2.16-2.02 (m, 8H). ³¹P NMR (162 MHz, CDCl₃) δ 29.96 (s). ee: >99% Retention time: 5.630 min |
| 182 | 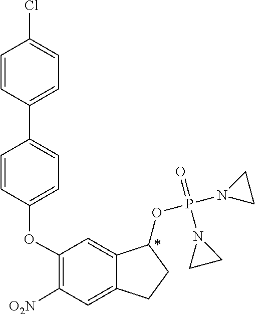 [SMILES: O=[N+]([O-])c1cc2c(cc1Oc1ccc(-c3ccc(Cl)cc3)cc1)C(OP(=O)(N1CC1)N1CC1)CC2] | LC-MS (ESI): m/z 534.1 [M + H]⁺; ¹H NMR (400 MHz, CDCl₃) δ 7.83 (s, 1H), 7.57-7.51 (m, 2H), 7.50- 7.45 (m, 2H), 7.43-7.38 (m, 2H), 7.24 (s, 1H), 7.12-7.06 (m, 2H), 5.96- 5.89 (m, 1H), 3.18-3.11 (m, 1H), 2.95-2.86 (m, 1H), 2.68-2.56 (m, 1H), 2.34- 2.24 (m, 1H), 2.20- 2.00 (m, 8H). ³¹P NMR (162 MHz, CDCl₃) δ 29.98 (s). ee: >99% Retention time: 6.716 min |
| 183 | 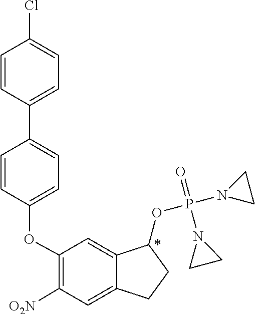 [SMILES: O=[N+]([O-])c1cc2c(cc1Oc1ccc(-c3ccc(Cl)cc3)cc1)C(OP(=O)(N1CC1)N1CC1)CC2] | LC-MS (ESI): m/z 534.2 [M + H]⁺; ¹H NMR (400 MHz, CDCl₃) δ 7.83 (s, 1H), 7.57-7.51 (m, 2H), 7.50- 7.45 (m, 2H), 7.43-7.38 (m, 2H), 7.24 (s, 1H), 7.12-7.07 (m, 2H), 5.96- 5.89 (m, 1H), 3.20-3.09 (m, 1H), 2.95-2.86 (m, 1H), 2.68-2.56 (m, 1H), 2.35-2.24 (m, 1H), 2.19-2.03 (m, 8H). ³¹P NMR (162 MHz, CDCl₃) δ 29.98 (s). ee: >99% Retention time: 4.883 min |
| 184 | 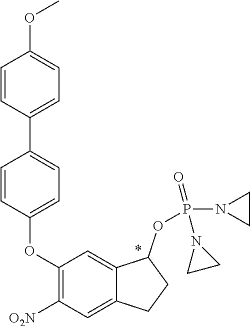 [SMILES: COc1ccc(-c2ccc(Oc3cc4c(cc3[N+](=O)[O-])CCC4OP(=O)(N3CC3)N3CC3)cc2)cc1] | LC-MS (ESI): m/z 508.1 [M + H]⁺; ¹H NMR (400 MHz, CDCl₃) δ 7.82 (s, 1H), 7.57-7.51 (m, 2H), 7.51- 7.46 (m, 2H), 7.21 (s, 1H), 7.13-7.07 (m, 2H), 7.01-6.95 (m, 2H), 5.96- 5.89 (m, 1H), 3.86 (s, 3H), 3.22-3.02 (m, 1H), 2.96-2.83 (m, 1H), 2.70- 2.53 (m, 1H), 2.34-2.21 (m, 1H), 2.17-2.01 (m, 8H). ³¹P NMR (162 MHz, CDCl₃) δ 29.97 (s). ee: >99% Retention time: 5.792 min |
| 185 | 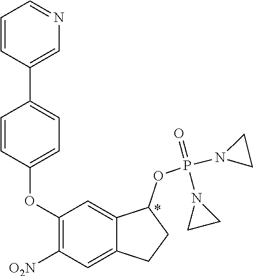 [SMILES: O=[N+]([O-])c1cc2c(cc1Oc1ccc(-c3cccnc3)cc1)C(OP(=O)(N1CC1)N1CC1)CC2] | LC-MS (ESI): m/z 508.1 [M + H]⁺; ¹H NMR (400 MHz, CDCl₃) δ 7.82 (s, 1H), 7.57-7.51 (m, 2H), 7.51- 7.45 (m, 2H), 7.21 (s, 1H), 7.12-7.07 (m, 2H), 7.02-6.94 (m, 2H), 6.05- 5.80 (m, 1H), 3.86 (s, 3H), 3.28-3.04 (m, 1H), 2.98-2.82 (m, 1H), 2.70- 2.55 (m, 1H), 2.33-2.24 (m, 1H), 2.20-1.99 (m, 8H). ³¹P NMR (162 MHz, CDCl₃) δ 29.97 (s). ee: >99% Retention time: 4.535 min |
| 186 | 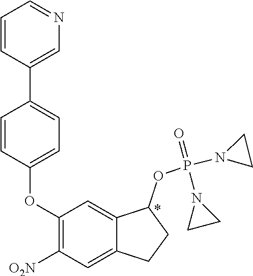 [SMILES: O=[N+]([O-])c1cc2c(cc1Oc1ccc(-c3cccnc3)cc1)C(OP(=O)(N1CC1)N1CC1)CC2] | LC-MS (ESI): m/z 508.1 [M + H]⁺; ¹H NMR (400 MHz, CDCl₃) δ 8.85-8.78 (m, 1H), 8.62-8.56 (m, 1H), 7.88-7.81 (m, 2H), 7.61-7.52 (m, 2H), 7.40-7.33 (m, 1H), 7.27 (s, 1H), 7.16-7.09 (m, 2H), 5.98-5.91 (m, 1H), 3.20-3.12 (m, 1H), 2.96-2.87 (m, 1H), 2.68-2.58 (m, 1H), 2.36-2.26 (m, 1H), 2.21-1.98 (m, 8H). ³¹P NMR (162 MHz, CDCl₃) δ 30.00 (s). ee: >99% Retention time: 5.846 min |
| 187 | 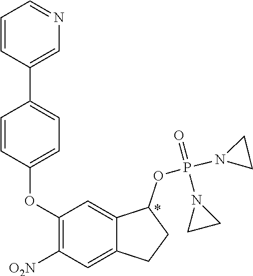 [SMILES: O=[N+]([O-])c1cc2c(cc1Oc1ccc(-c3cccnc3)cc1)C(OP(=O)(N1CC1)N1CC1)CC2] | LC-MS (ESI): m/z 479.1 [M + H]⁺; ¹H NMR (400 MHz, CDCl₃) δ 8.84- 8.79 (m, 1H), 8.59 (dd, J = 4.8, 1.6 Hz, 1H), 7.87-7.82 (m, 2H), 7.60-7.54 (m, 2H), 7.40-7.35 (m, 1H), 7.27 (s, 1H), 7.15-7.10 (m, 2H), 5.97-5.90 (m, 1H), 3.20-3.11 (m, 1H), 2.96- 2.88 (m, 1H), 2.68-2.58 (m, 1H), 2.36-2.25 (m, 1H), 2.20-2.03 (m, 8H). ³¹P NMR (162 MHz, CDCl₃) δ 29.99 (s). ee: >99% Retention time: 4.781 min |
| 188 | 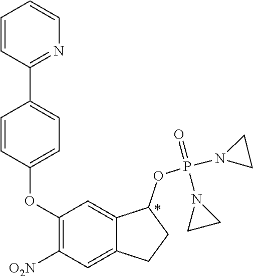 [SMILES: O=[N+]([O-])c1cc2c(cc1Oc1ccc(-c3ccccn3)cc1)C(OP(=O)(N1CC1)N1CC1)CC2] | LC-MS (ESI): m/z 479.1 [M + H]⁺; ¹H NMR (400 MHz, CDCl₃) δ 8.67 (d, J = 4.4 Hz, 1H), 8.01 (d, J = 8.7 Hz, 2H), 7.83 (s, 1H), 7.79-7.72 (m, 1H), 7.69 (d, J = 7.9 Hz, 1H), 7.26- 7.19 (m, 2H), 7.12 (d, J = 8.7 Hz, 2H), 6.03-5.81 (m, 1H), 3.21-3.08 (m, 1H), 2.95-2.81 (m, 1H), 2.68-2.55 (m, 1H), 2.34-2.26 (m, 1H), 2.17- 2.03 (m, 8H). ³¹P NMR (162 MHz, CDCl₃) δ 29.94 (s). ee: >99% Retention time: 5.393 min |
| 189 | 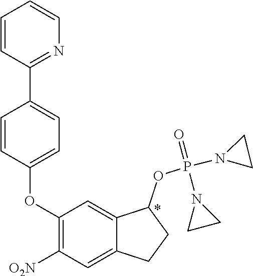 [SMILES: O=[N+]([O-])c1cc2c(cc1Oc1ccc(-c3ccccn3)cc1)C(OP(=O)(N1CC1)N1CC1)CC2] | LC-MS (ESI): m/z 479.1 [M + H]⁺; ¹H NMR (400 MHz, CDCl₃) δ 8.67 (d, J = 4.7 Hz, 1H), 8.01 (d, J = 8.8 Hz, 2H), 7.83 (s, 1H), 7.78-7.72 (m, 1H), 7.70 (d, J = 7.9 Hz, 1H), 7.26- 7.20 (m, 2H), 7.12 (d, J = 8.8 Hz, 2H), 5.97-5.85 (m, 1H), 3.18-3.10 (m, 1H), 2.96-2.86 (m, 1H), 2.66-2.55 (m, 1H), 2.35- 2.24 (m, 1H), 2.16- 2.00 (m, 8H). ³¹P NMR (162 MHz, CDCl₃) δ 29.94 (s). ee: >99% Retention time: 3.960 min |
| 190 | 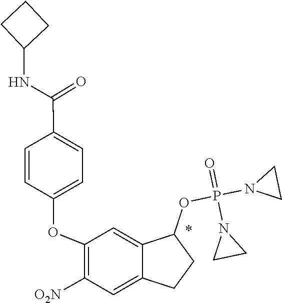 [SMILES: O=C(NC1CCC1)c1ccc(Oc2cc3c(cc2[N+](=O)[O-])CCC3OP(=O)(N2CC2)N2CC2)cc1] | LC-MS (ESI): m/z 499.1 [M + H]⁺; ¹H NMR (400 MHz, CDCl₃) δ 7.84 (s, 1H), 7.76-7.73 (m, 2H), 7.27 (s, 1H), 7.04- 6.99 (m, 2H), 6.18 (d, J = 7.6 Hz, 1H), 5.96-5.91 (m, 1H), 4.61- 4.55 (m, 1H), 3.20-3.13 (m, 1H), 2.98-2.87 (m, 1H), 2.70-2.58 (m, 1H), 2.50-2.39 (m, 2H), 2.37-2.26 (m, 1H), 2.21-2.19 (m, 1H), 2.17- 2.06 (m, 7H), 2.00-1.91 (m, 2H), 1.83-1.74 (m, 2H). ³¹P NMR (162 MHz, CDCl₃) δ 29.99 (s). ee: 98% Retention time: 4.788 min |
| 191 | 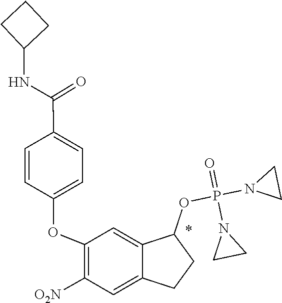 [SMILES: O=C(NC1CCC1)c1ccc(Oc2cc3c(cc2[N+](=O)[O-])CCC3OP(=O)(N2CC2)N2CC2)cc1] | LC-MS (ESI): m/z 499.1 [M + H]⁺; ¹H NMR (400 MHz, CDCl₃) δ 7.84 (s, 1H), 7.76-7.73 (m, 2H), 7.27 (s, 1H), 7.04-6.99 (m, 2H), 6.18 (d, J = 7.6 Hz, 1H), 5.96-5.91 (m, 1H), 4.61-4.55 (m, 1H), 3.20-3.13 (m, 1H), 2.98-2.87 (m, 1H), 2.70-2.58 (m, 1H), 2.50-2.39 (m, 2H), 2.37- 2.26 (m, 1H), 2.21-2.19 (m, 1H), 2.17- 2.06 (m, 7H), 2.00-1.91 (m, 2H), 1.83-1.74 (m, 2H). ³¹P NMR (162 MHz, CDCl₃) δ 29.99 (s). ee: >99% Retention time: 4.019 min |
| 192 | 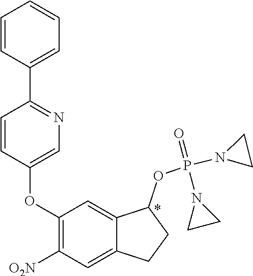 [SMILES: O=[N+]([O-])c1cc2c(cc1Oc1ccc(-c3ccccc3)nc1)C(OP(=O)(N1CC1)N1CC1)CC2] | LC-MS (ESI): m/z 479.1 [M + H]⁺; ¹H NMR (400 MHz, CDCl₃) δ 8.40 (d, J = 2.8 Hz, 1H), 7.91-7.84 (m, 2H), 7.79 (s, 1H), 7.66 (d, J = 8.7 Hz, 1H), 7.40 (t, J = 7.3 Hz, 2H), 7.37-7.31 (m, 2H), 7.22 (s, 1H), 5.87 (dd, J = 12.6, 7.2 Hz, 1H), 3.13- 3.03 (m, 1H), 2.92-2.79 (m, 1H), 2.63-2.49 (m, 1H), 2.29-2.21 (m, 1H), 2.12-1.98 (m, 8H). ³¹P NMR (162 MHz, CDCl₃) δ 29.96 (s). ee: >99% Retention time: 4.976 min |
| 193 | 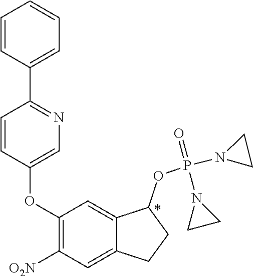 [SMILES: O=[N+]([O-])c1cc2c(cc1Oc1ccc(-c3ccccc3)nc1)C(OP(=O)(N1CC1)N1CC1)CC2] | LC-MS (ESI): m/z 479.1 [M + H]⁺; ¹H NMR (400 MHz, CDCl₃) δ 8.40 (d, J = 2.8 Hz, 1H), 7.91-7.84 (m, 2H), 7.79 (s, 1H), 7.66 (d, J = 8.7 Hz, 1H), 7.40 (t, J = 7.3 Hz, 2H), 7.37-7.31 (m, 2H), 7.22 (s, 1H), 5.87 (dd, J = 12.6, 7.2 Hz, 1H), 3.13- 3.03 (m, 1H), 2.92-2.79 (m, 1H), 2.63-2.49 (m, 1H), 2.29-2.21 (m, 1H), 2.12-1.98 (m, 8H). ³¹P NMR (162 MHz, CDCl₃) δ 29.96 (s). ee: >99% Retention time: 3.853 min |
| 194 | 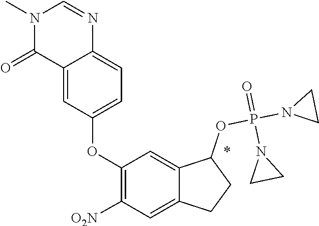 [SMILES: Cn1cnc2ccc(Oc3cc4c(cc3[N+](=O)[O-])CCC4OP(=O)(N3CC3)N3CC3)cc2c1=O] | LC-MS (ESI): m/z 484.1 [M + H]⁺; ¹H NMR (400 MHz, CDCl₃) δ 8.01-7.93 (m, 1H), 7.89-7.81 (m, 1H), 7.76-7.68 (m, 1H), 7.68-7.63 (m, 1H), 7.52-7.45 (m, 1H), 7.34-7.28 (m, 1H), 5.96-5.85 (m, 1H), 3.58-3.51 (m, 3H), 3.20-3.09 (m, 1H), 2.99- 2.88 (m, 1H), 2.66-2.54 (m, 1H), 2.36-2.26 (m, 1H), 2.19-2.04 (m, 8H). ³¹P NMR (162 MHz, CDCl₃) δ 29.76 (s). ee: >99% Retention time: 5.884 min |
| 195 | 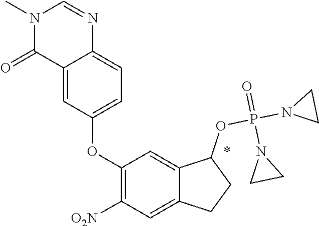 [SMILES: Cn1cnc2ccc(Oc3cc4c(cc3[N+](=O)[O-])CCC4OP(=O)(N3CC3)N3CC3)cc2c1=O] | LC-MS (ESI): m/z 484.1 [M + H]⁺; ¹H NMR (400 MHz, CDCl₃) δ 8.03-7.97 (m, 1H), 7.91-7.85 (m, 1H), 7.78-7.71 (m, 1H), 7.70-7.64 (m, 1H), 7.54-7.48 (m, 1H), 7.38-7.32 (m, 1H), 5.96-5.89 (m, 1H), 3.57 (s, 3H), 3.22-3.14 (m, 1H), 2.97-2.90 (m, 1H), 2.69-2.60 (m, 1H), 2.35-2.29 (m, 1H), 2.17-2.04 (m, 8H). ³¹P NMR (162 MHz, CDCl₃) δ 29.76 (s). ee: >99% Retention time: 4.878 min |
| 196 | 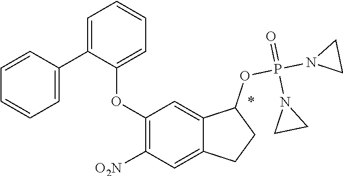 [SMILES: O=[N+]([O-])c1cc2c(cc1Oc1ccccc1-c1ccccc1)C(OP(=O)(N1CC1)N1CC1)CC2] | LC-MS (ESI): m/z 478.2 [M + H]⁺; ¹H NMR (400 MHz, CDCl₃) δ 7.70 (s, 1H), 7.57-7.45 (m, 3H), 7.39- 7.27 (m, 5H), 7.11-7.06 (m, 1H), 6.91 (s, 1H), 5.84-5.75 (m, 1H), 3.08- 2.91 (m, 2H), 2.85-2.74 (m, 1H), 2.58-2.47 (m, 1H), 2.21-1.99 (m, 9H). ³¹P NMR (162 MHz, CDCl₃) δ 29.78 (s). ee: >99% Retention time: 3.857 min |
| 197 | 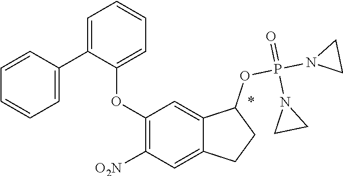 [SMILES: O=[N+]([O-])c1cc2c(cc1Oc1ccccc1-c1ccccc1)C(OP(=O)(N1CC1)N1CC1)CC2] | LC-MS (ESI): m/z 478.2 [M + H]⁺; ¹H NMR (400 MHz, CDCl₃) δ 7.71 (s, 1H), 7.55-7.47 (m, 3H), 7.38- 7.27 (m, 5H), 7.10-7.06 (m, 1H), 6.91 (s, 1H), 5.83- 5.76 (m, 1H), 3.09- 2.97 (m, 1H), 2.85-2.73 (m, 1H), 2.58-2.47 (m, 1H), 2.24-1.97 (m, 9H). ³¹P NMR (162 MHz, CDCl₃) δ 29.80 (s). ee: >99% Retention time: 3.283 min |
| 198 | 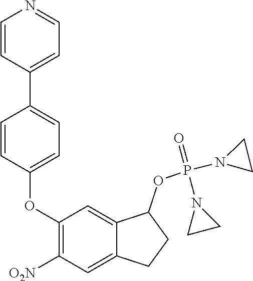 [SMILES: O=[N+]([O-])c1cc2c(cc1Oc1ccc(-c3ccncc3)cc1)C(OP(=O)(N1CC1)N1CC1)CC2] | LC-MS (ESI): m/z 479.1 [M + H]⁺; ¹H NMR (400 MHz, CDCl₃) δ 8.67 (d, J = 6.2 Hz, 2H), 7.86 (s, 1H), 7.68-7.63 (m, 2H), 7.56 (d, J = 6.0 Hz, 2H), 7.32 (s, 1H), 7.15-7.09 (m, 2H), 5.99-5.89 (m, 1H), 3.22-3.13 (m, 1H), 2.99-2.88 (m, 1H), 2.69- 2.59 (m, 1H), 2.38-2.27 (m, 1H), 2.22-2.02 (m, 8H). ³¹P NMR (162 MHz, CDCl₃) δ 30.00 (s). |

TABLE 5
Characterization of compounds (see General Procedure 7)

| Compound | | |
|---|---|---|
| No. | Structure | LCMS; ¹H NMR (ppm); Retention time |
| 199 | 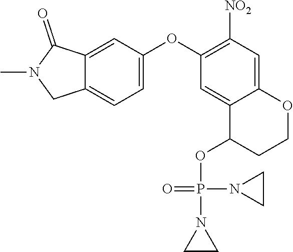 [SMILES: CN1Cc2ccc(Oc3cc4c(cc3[N+](=O)[O-])OCCC4OP(=O)(N3CC3)N3CC3)cc2C1=O] | LCMS(ESI): m/z 487.1[M + H]+; ¹H NMR (400 MHz, CDCl₃) δ 7.49 (d, J = 5.9 Hz, 1H), 7.41 (d, J = 8.2 Hz, 1H), 7.34 (s, 1H), 7.24 (d, J = 2.4 Hz, 1H), 7.19 (d, J = 2.3 Hz, 1H), 5.65-5.45 (m, 1H), 4.42-4.30 (m, 4H), 3.18 (s, 3H), 2.34-2.26 (m, 2H), 2.25-2.02 (m, 8H). ³¹P NMR(162 MHz, CDCl₃) δ 29.75 (s) |
| 200 | 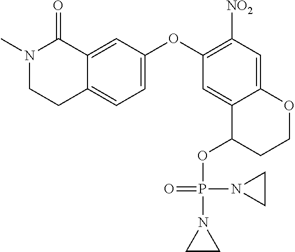 [SMILES: CN1CCc2ccc(Oc3cc4c(cc3[N+](=O)[O-])OCCC4OP(=O)(N3CC3)N3CC3)cc2C1=O] | LCMS(ESI): m/z 501.1[M + H]⁺; ¹H NMR (400 MHz, CDCl₃) δ 7.54 (d, J = 2.6 Hz, 1H), 7.46 (d, J = 4.5 Hz, 1H), 7.25 (s, 1H), 7.16 (d, J = 8.2 Hz, 1H), 7.10 (dd, J = 8.2, 2.7 Hz, 1H), 5.62- 5.44 (m, 1H), 4.40-4.27 (m, 2H), 3.55 (t, J = 6.7 Hz, 2H), 3.12 (s, 3H), 2.98 (t, J = 6.7 Hz, 2H), 2.36-2.25 (m, 2H), 2.25-2.03 (m, 8H) ³¹P NMR(162 MHz, CDCl₃) δ 29.62 (s). |
| 201 | 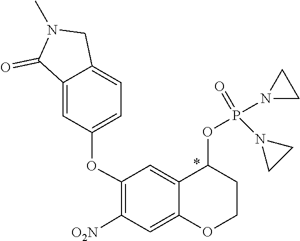 [SMILES: CN1Cc2ccc(Oc3cc4c(cc3[N+](=O)[O-])OCCC4OP(=O)(N3CC3)N3CC3)cc2C1=O] | LCMS(ESI): m/z 487.1[M + H]⁺; ¹H NMR (400 MHz, CDCl₃) δ 7.52-7.46 (m, 1H),, 7.44-7.32 (m, 2H), 7.24-7.17 (m, 2H), 5.57-5.37 (m, 1H), 4.43-4.30 (m, 4H), 3.18 (s, 3H), 2.35-1.95 (m, 10H). ³¹P NMR (162 MHz, CDCl₃) δ 29.74 (s). |
| 202 | 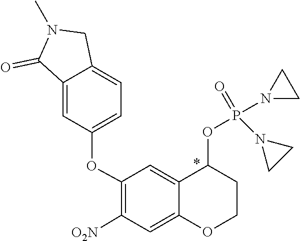 [SMILES: CN1Cc2ccc(Oc3cc4c(cc3[N+](=O)[O-])OCCC4OP(=O)(N3CC3)N3CC3)cc2C1=O] | LCMS(ESI): m/z 487.2[M + H]⁺; ¹H NMR (400 MHz, CDCl₃) δ 7.52-7.46 (m, 1H), 7.43-7.37 (m, 1H), 7.34-7.29 (m, 1H), 7.26-7.22 (m, 1H), 7.22-7.16 (m, 1H), 5.60-5.52 (m, 1H), 4.44-4.27 (m, 4H), 3.18 (s, 3H), 2.35-2.25 (m, 2H), 2.23-2.01 (m, 8H). ³¹P NMR (162 MHz, CDCl₃) δ 29.76 (s). ee: 99.5% Retention time: 5.499 min; Column: ChiralCel OD, 250 × 4.6 mm I.D., 5 μm, Mobile phase: A for CO₂ and B for EtOH(0.05% DEA), Gradient: 8 min @ B 30%, Flow rate: 2.0 mL/min, Back pressure: 100 bar, Column temperature: 35° C. |
| 203 | 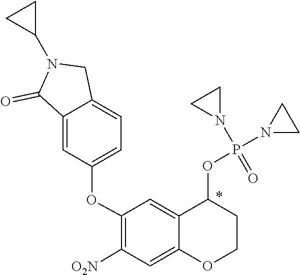 [SMILES: O=C1c2cc(Oc3cc4c(cc3[N+](=O)[O-])OCCC4OP(=O)(N3CC3)N3CC3)ccc2CN1C1CC1] | LCMS(ESI): m/z 513.2[M + H]⁺; ¹H NMR (400 MHz, CDCl₃) δ 7.49 (s, 1H), 7.38 (d, J = 8.3 Hz, 1H), 7.32 (s, 1H), 7.26-7.22 (m, 1H), 7.18 (d, J = 2.3 Hz, 1H), 5.58-5.51 (m, 1H), 4.42-4.30 (m, 2H), 4.29 (s, 2H), 2.95-2.89 (m, 1H), 2.35-2.27 (m, 2H), 2.22-2.05 (m, 8H), 1.00-0.71 (m, 4H). ³¹P NMR (162 MHz, CDCl₃) δ 29.74 (s). ee: >99% Retention time: 2.482 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |
| 204 | 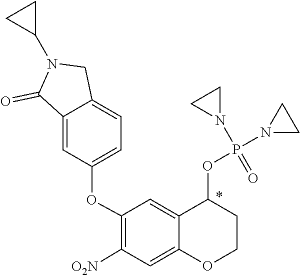 [SMILES: O=C1c2cc(Oc3cc4c(cc3[N+](=O)[O-])OCCC4OP(=O)(N3CC3)N3CC3)ccc2CN1C1CC1] | LCMS(ESI): m/z 513.2[M + H]⁺; ¹H NMR (400 MHz, CDCl₃) δ 7.49 (s, 1H), 7.38 (d, J = 8.3 Hz, 1H), 7.32 (s, 1H), 7.26-7.23 (m, 1H), 7.18 (d, J = 2.3 Hz, 1H), 5.60-5.49 (m, 1H), 4.43-4.31 (m, 2H), 4.29 (s, 2H), 2.96-2.87 (m, 1H), 2.36-2.25 (m, 2H), 2.22-1.97 (m, 8H), 0.94-0.81 (m, 4H). ³¹P NMR (162 MHz, CDCl₃) δ 29.74 (s). ee: >99% Retention time: 1.536 min; Column: ChiralPak IH, 100 × 4.6 mm I.D., 5 μm; Mobile phase: A for CO₂ and B for methanol (0.05% DEA); Gradient: 8 min @ 30% B; Flow rate: 2.5 mL/min; Column temperature: 40° C. |

TABLE 6
Characterization of compounds (see General Procedures 2-4)

| Com- | | |
|---|---|---|
| pound | | |
| No. | Structure | LCMS; ¹H NMR (ppm); Retention time |
| 205 | 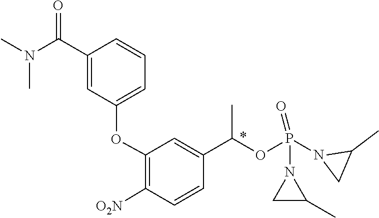 | LCMS(ESI): m/z 489.1 [M + H]⁺; ¹H NMR (400 MHz, CDCl₃) δ 8.06-7.89 (m, 1H), 7.44-7.38 (m, 1H), 7.31-7.28 (m, 1H), 7.27-7.26 (m, 1H), 7.22-7.21 (m, 1H), 7.12-7.08 (m, 1H), 7.07-7.05 (m, 1H), 5.63-5.57 (m, 1H), 3.09 (s, 3H), 2.98 (s, 3H) ,2.59-2.37 (m, 2H), 2.35-2.14 (m, 2H), 1.91-1.69 (m, 2H), 1.62-1.47 (m, 3H), 1.30-1.05 (m, 6H). ³¹P NMR (162 MHz, CDCl₃) δ 31.62-26.12 (m). |
| 206 | 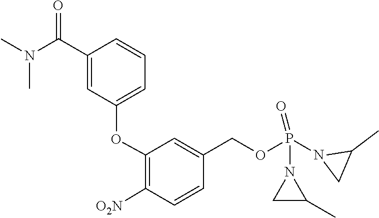 | LCMS(ESI): m/z 475.2 [M + H]⁺; ¹H NMR (400 MHz, CDCl₃) ) δ 7.99 (dd, J = 8.4, 1.8 Hz, 1H), 7.45-7.39 (m, 1H), 7.28 (d, J = 1.9 Hz, 1H), 7.23 (d, J = 7.7 Hz, 1H), 7.10-7.05 (m, 3H), 5.13 (dd, J = 7.6, 3.8 Hz, 2H), 3.09 (s, 3H), 2.98 (s, 3H), 2.54-2.52 (m, 2H), 2.38-2.24 (m, 2H), 1.90-1.88 (m, 1H), 1.82-1.80 (m, 1H), 1.28- 1.24 (m, 3H), 1.23-1.20 (m, 3H). ³¹P NMR (162 MHz, CDCl₃) 6 29.83 (t, J = 26.0 Hz). |
| 207 | 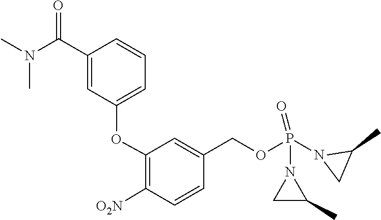 | LCMS(ESI): m/z 475.2 [M + H]⁺; ¹H NMR (400 MHz, CDCl₃) δ 7.99 (d, J = 8.4 Hz, 1H), 7.45-7.41(m, 1H), 7.29-7.21 (m, 2H), 7.11-7.05 (m, 3H), 5.13 (d, J = 7.6 Hz, 2H), 3.10 (s, 3H), 2.98 (s, 3H), 2.57-2.50 (m, 2H), 2.35-2.30 (m, 2H), 1.91-1.79 (m, 2H), 1.29-1.25 (m, 3H), 1.23-1.22 (m, 3H). ³¹P NMR (162 MHz, CDCl₃) ) δ 29.69 (s). |
| 208 | 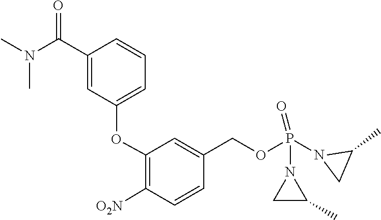 | LCMS(ESI): m/z 475.2 [M + H]⁺; ¹H NMR (400 MHz, CDCl₃) δ 7.99 (d, J = 8.4 Hz, 1H), 7.44-7.40 (m, 1H), 7.28-7.22 (m, 2H), 7.10-7.07 (m, 3H), 5.13 (d, J = 7.6 Hz, 2H), 3.09 (s, 3H), 2.98 (s, 3H), 2.60-2.45 (m, 2H), 2.35-2.28 (m, 2H), 1.91- 1.79 (m, 2H), 1.27-1.25 (m, 3H), 1.23-1.21 (m, 3H) ³¹P NMR (162 MHz, CDCl₃) ) δ 29.67 (s) |
| 209 | 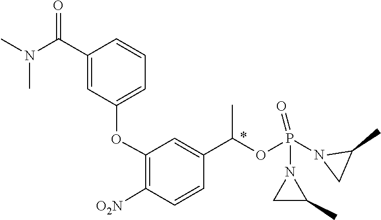 | LCMS(ESI): m/z 489.2 [M + H]⁺; ¹H NMR (400 MHz, CDCl₃) δ 7.99 (d, J = 8.5 Hz, 1H), 7.44-7.38 (m, 1H), 7.31-7.28 (m, 1H), 7.24-7.22 (m, 1H), 7.11-7.05 (m, 3H), 5.63-5.55 (m, 1H), 3.10 (s, 3H), 2.99 (s, 3H), 2.54-2.40 (m, 2H), 2.35-2.18 (m, 2H), 1.91-1.87 (m, 1H), 1.73-1.56 (m, 1H), 1.57 (d, J = 6.6 Hz, 3H), 1.28-1.20 (m, 3H), 1.18-1.13 (m, 3H). ³¹P NMR (162 MHz, CDCl₃) δ 29.06 (s). |
| 210 | 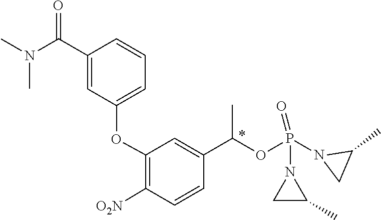 | LCMS(ESI): m/z 489.2 [M + H]⁺; ¹H NMR (400 MHz, CDCl₃) δ 7.98 (d, J = 8.4 Hz, 1H), 7.43-7.39 (m, 1H), 7.28-7.21 (m, 2H), 7.10-7.04 (m, 3H), 5.60-5.57 (m, 1H), 3.09 (s, 3H), 2.98 (s, 3H), 2.58-2.39 (m, 2H), 2.32-2.25 (m, 1H), 2.20-2.14 (m, 1H), 1.81-1.70 (m, 2H), 1.56 (d, J = 6.5 Hz, 3H), 1.27-1.19 (m, 3H), 1.17-1.12 (m, 3H). ³¹P NMR (162 MHz, CDCl₃) δ 29.07 (s). |
| 211 | 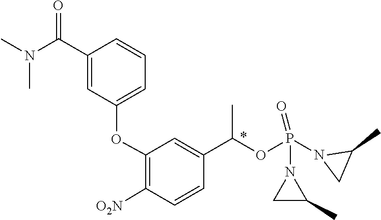 | LCMS(ESI): m/z 489.2 [M + H]⁺; ¹H NMR (400 MHz, CDCl₃) δ 7.98 (d, J = 8.4 Hz, 1H), 7.45-7.36 (m, 1H), 7.31-7.27 (m, 1H), 7.24-7.17 (m, 1H), 7.10 (d, J = 1.6 Hz, 1H), 7.07-7.03 (m, 2H), 5.65-5.52 (m, 1H), 3.09 (s, 3H), 2.98 (s, 3H), 2.55- 2.37 (m, 2H), 2.36-2.25 (m, 1H), 2.24-2.15 (m, 1H), 1.92-1.83 (m, 1H), 1.75-1.65 (m, 1H), 1.56 (d, J = 6.6 Hz, 3H), 1.29-1.07 (m, 6H). ³¹P NMR (162 MHz, CDCl₃) δ 29.03 (s) |
| 212 | 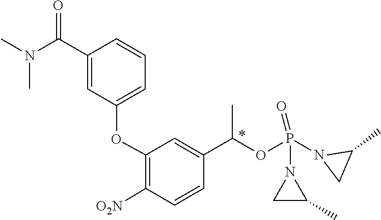 | LCMS(ESI): m/z 489.2 [M + H]⁺; ¹H NMR (400 MHz, CDCl₃) ) δ 7.91 (d, J = 8.4 Hz, 1H), 7.39-7.30 (m, 1H), 7.23-7.20 (m, 1H), 7.19-7.11 (m, 1H), 7.07-6.92 (m, 3H), 5.55-5.46 (m, 1H), 3.06-2.83 (m, 6H), 2.53-2.28 (m, 2H), 2.26-2.05 (m, 2H), 1.75- 1.62 (m, 2H), 1.53-1.44 (d, J = 6.4 Hz, 3H), 1.22-0.97 (m, 6H). ³¹P NMR (162 MHz, CDCl₃) δ 29.10 (s) |
| 213 | 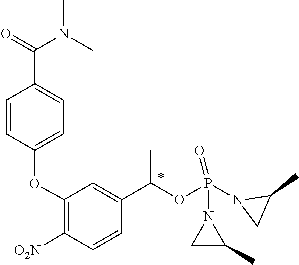 [SMILES: CC(OP(=O)(N1C[C@@H]1C)N1C[C@@H]1C)c1ccc([N+](=O)[O-])c(Oc2ccc(C(=O)N(C)C)cc2)c1] | LCMS(ESI): m/z 489.3 [M + H]+; ¹H NMR (400 MHz, CDCl3) δ 7.99 (d, J = 8.5 Hz, 1H), 7.46 (d, J = 8.6 Hz, 2H), 7.31-7.28 (m, 1H), 7.11 (d, J = 1.4 Hz, 1H), 7.03 (d, J = 8.6 Hz, 2H), 5.66-5.56 (m, 1H), 3.11 (s, 3H), 3.03 (s, 3H), 2.60-2.38 (m, 2H), 2.32 (dd, J = 17.3, 5.9 Hz, 1H), 2.20 (dd,J = 17.7, 5.9 Hz, 1H), 1.89 (dd, J = 13.5, 3.3 Hz, 1H), 1.70 (dd, J = 13.2, 3.2 Hz, 1H), 1.57 (d, J = 6.5 Hz, 3H), 1.21 (d, J = 5.4 Hz, 3H), 1.14 (d, J = 5.4 Hz, 3H). ³¹P NMR (162 MHz, CDCl₃) δ 29.11 (s). |
| 214 | 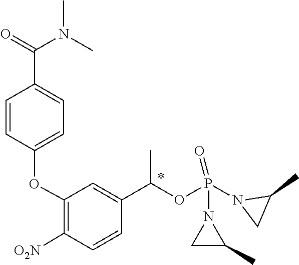 [SMILES: CC(OP(=O)(N1C[C@@H]1C)N1C[C@@H]1C)c1ccc([N+](=O)[O-])c(Oc2ccc(C(=O)N(C)C)cc2)c1] | LCMS(ESI): m/z 489.2 [M + H]⁺; ¹H NMR (400 MHz, CDCl₃) δ 7.98 (d, J = 8.4 Hz, 1H), 7.45 (d, J = 8.6 Hz, 2H), 7.28 (s, 1H), 7.10 (s, 1H), 7.03 (d, J = 8.6 Hz, 2H), 5.64-5.53 (m, 1H), 3.10 (s, 3H), 3.03 (s, 3H), 2.56-2.48 (m, 1H), 2.47-2.35 (m, 1H), 2.34-2.11 (m, 2H), 1.83-1.66 (m, 2H), 1.56 (d, J = 6.5 Hz, 3H), 1.25 (d, J = 5.3 Hz, 3H), 1.16 (d, J = 5.3 Hz, 3H). ³¹P NMR (162 MHz, CDCl₃) δ 29.12 (s). |
| 215 | 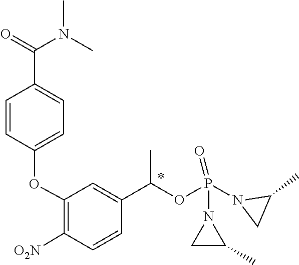 [SMILES: CC(OP(=O)(N1C[C@H]1C)N1C[C@H]1C)c1ccc([N+](=O)[O-])c(Oc2ccc(C(=O)N(C)C)cc2)c1] | LCMS(ESI): m/z 489.2 [M + H]⁺; ¹H NMR (400 MHz, CDCl₃) δ 7.95-7.87 (m, 1H), 7.44-7.33 (m, 2H), 7.25-7.15 (m, 2H), 7.06-6.91 (m, 2H), 5.58-5.46 (m, 1H), 3..03 (s, 3H), 2.96 (s, 3H), 2.53-2.30 (m, 2H), 2.27-2.05 (m, 2H), 1.75-1.60 (m, 2H), 1.51-1.44 (m, 3H), 1.30-0.98 (m, 6H). ³¹P NMR (162 MHz, CDCl₃) δ 29.16 (s). |
| 216 | 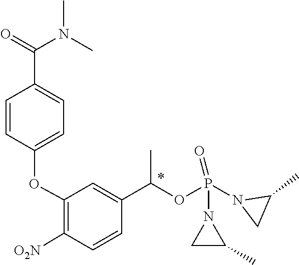 [SMILES: CC(OP(=O)(N1C[C@H]1C)N1C[C@H]1C)c1ccc([N+](=O)[O-])c(Oc2ccc(C(=O)N(C)C)cc2)c1] | LCMS(ESI): m/z 489.1 [M + H]⁺; ¹H NMR (400 MHz, CDCl₃) δ 8.06-7.94 (m, 1H), 7.52-7.40 (m, 2H), 7.39-7.29 (m, 1H), 7.17-7.08 (m, 1H), 7.08-6.99 (m, 2H), 5.79-5.44 (m, 1H), 3.11 (s, 3H), 3.04 (s, 3H), 2.58-2.37 (m, 2H), 2.37-2.27 (m, 1H), 2.26-2.15 (m, 1H), 1.93-1.82 (m, 1H), 1.77-1.66 (m, 1H), 1.57 (d, J = 6.5 Hz, 3H), 1.25-1.09 (m, 6H) ³¹P NMR (162 MHz, CDCl₃) δ 29.10 (s) |
| 217 | 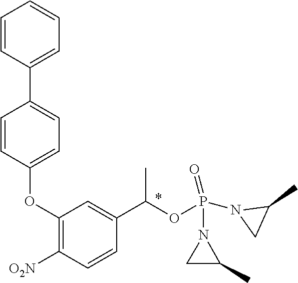 [SMILES: CC(OP(=O)(N1C[C@@H]1C)N1C[C@@H]1C)c1ccc([N+](=O)[O-])c(Oc2ccc(-c3ccccc3)cc2)c1] | LCMS(ESI): m/z 494.2 [M + H]⁺; ¹H NMR (400 MHz, CDCl₃) δ 7.98 (d, J = 8.4 Hz, 1H), 7.61-7.56 (m, 4H), 7.47-7.42 (m, 2H), 7.38-7.32 (m, 1H), 7.25-7.22 (m, 1H), 7.14- 7.08 (m, 3H), 5.61- 5.54 (m, 1H), 2.5-2.39 (m, 2H), 2.38-2.34 (m, 1H), 2.21-2.15 (m, 1H), 1.89-1.85 (m, 1H), 1.68-1.64 (m, 1H), 1.57 (d, J = 6.5 Hz, 3H), 1.18 (d, J = 5.4 Hz, 3H), 1.13 (d, J = 5.3 Hz, 3H). ³¹P NMR (162 MHz, CDCl₃) δ 29.07 (s). |
| 218 | 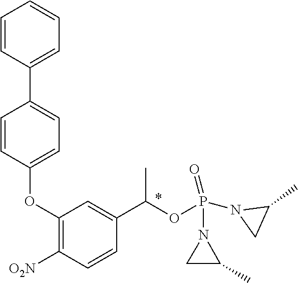 [SMILES: CC(OP(=O)(N1C[C@H]1C)N1C[C@H]1C)c1ccc([N+](=O)[O-])c(Oc2ccc(-c3ccccc3)cc2)c1] | LCMS(ESI): m/z 494.2 [M + H]⁺; ¹H NMR (400 MHz, DMSO-d₆) δ 8.12 (d, J = 8.4 Hz, 1H), 7.74 (d, J = 8.3 Hz, 2H), 7.67 (d, J = 7.9 Hz, 2H), 7.47 (t, J = 7.5 Hz, 2H), 7.41-7.35 (m, 2H), 7.23-7.15 (m, 3H), 5.62-5.49 (m, 1H), 2.41-2.36 (m, 1H), 2.27- 2.21 (m, 1H), 2.13-2.07 (m, 1H), 2.03-1.97 (m, 1H), 1.5-1.69 (m, 2H), 1.48 (d, J = 6.4 Hz, 3H), 1.12 (d, J = 5.3 Hz, 3H), 1.00 (d, J = 5.3 Hz, 3H). ³¹P NMR (162 MHz, DMSO-d₆) δ 28.44 (s). |
| 219 | 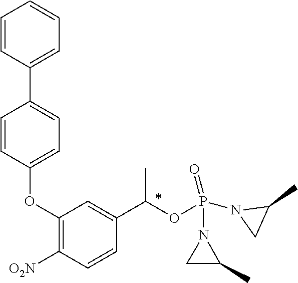 [SMILES: CC(OP(=O)(N1C[C@@H]1C)N1C[C@@H]1C)c1ccc([N+](=O)[O-])c(Oc2ccc(-c3ccccc3)cc2)c1] | LCMS(ESI): m/z 494.3 [M + H]⁺; ¹H NMR (400 MHz, CDCl₃) δ 7.98 (d, J = 2.9 Hz, 1H), 7.61-7.56 (m, 4H), 7.47-7.7.43(m, 2H), 7.37-7.34 (m, 1H), 7.22 (dd, J = 8.5, 1.6 Hz, 1H), 7.12-7.10 (m, 3H), 5.62-5.55 (m, 1H), 2.57-2.47 (m, 1H), 2.44-2.35 (m, 1H), 2.31-2.25 (m, 1H), 2.17-2.12 (m, 1H), 1.78-1.68 (m, 2H), 1.56 (d, J = 6.5 Hz, 3H), 1.25 (d, J = 5.4 Hz, 3H), 1.14 (d, J = 5.4 Hz, 3H). ³¹P NMR (162 MHz, CDCl₃) δ 29.13 (s). |
| 220 | 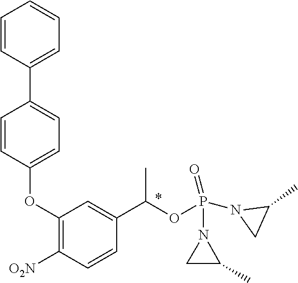 [SMILES: CC(OP(=O)(N1C[C@H]1C)N1C[C@H]1C)c1ccc([N+](=O)[O-])c(Oc2ccc(-c3ccccc3)cc2)c1] | LCMS(ESI): m/z 494.2 [M + H] ; ¹H NMR (400 MHz, CDCl₃) δ 7.98 (d, J = 8.4 Hz, 1H), 7.65-7.52 (m, 4H), 7.44 (t, J = 7.6 Hz, 2H), 7.35 (t, J = 7.3 Hz, 1H), 7.30-7.20 (m, 2H), 7.12-7.10 (m, 3H), 5.64-5.51 (m, 1H), 2.55-2.36 (m, 2H), 2.33-2.28 (m, 1H), 2.21-2.14 (m, 1H), 1.89-1.85 (m, 1H), 1.668-1.64(m, 1H), 1.56 (d, J = 6.5 Hz, 3H), 1.18 (d, J = 4.7 Hz, 3H), 1.13 (d, J = 5.2 Hz, 3H). ³¹P NMR (162 MHz, CDCl₃) δ 29.03 (s). |
| 221 | 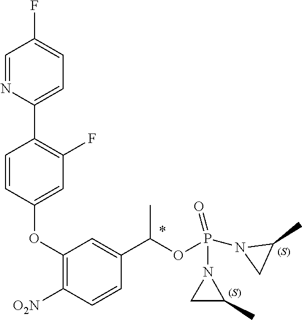 | LCMS(ESI): m/z 531.3 [M + H]⁺; ¹H NMR (400 MHz, CDCl₃) δ 8.56 (d, J = 2.9 Hz, 1H), 8.05-7.96 (m, 2H), 7.81-7.78 (m, 1H), 7.52-7.45 (m, 1H), 7.33 (dd, J = 8.5, 1.6 Hz, 1H), 7.20 (d, J = 1.5 Hz, 1H), 6.91 (dd, J = 8.7, 2.4 Hz, 1H), 6.82 (dd, J = 11.9, 2.4 Hz, 1H), 5.67-5.57 (m, 1H), 2.55-2.40 (m, 2H), 2.35-2.29 (m, 1H), 2.24-2.18 (m, 1H), 1.91-1.87 (m, 1H), 1.72-1.68 (m, 1H), 1.58 (d, J = 6.5 Hz, 3H), 1.20 (d, J = 5.4 Hz, 3H), 1.14 (d, J = 5.4 Hz, 3H). ³¹P NMR (162 MHz, CDCl₃) δ 29.19 (s). ¹⁹F NMR (377 MHz, CDCl₃) δ −113.37 (s), −128.44 (s). |
| 222 | 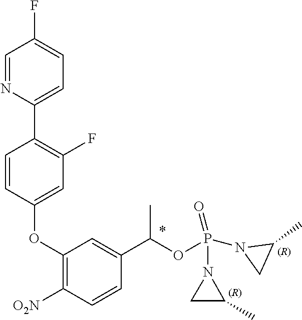 | LCMS(ESI): m/z 531.2 [M + H]⁺; ¹H NMR (400 MHz, CDCl₃) δ 8.56 (d, J = 2.9 Hz, 1H), 8.05-7.96 (m, 2H), 7.82-7.78 (m, 1H), 7.54-7.43 (m, 1H), 7.32-7.29 (m, 1H), 7.20-7.18 (m, 1H), 6.94-6.79 (m, 2H), 5.67-5.61 (m, 1H), 2.64-2.38 (m, 2H), 2.35- 2.26 (m, 1H), 2.23-2.14 (m, 1H), 1.83-1.76 (m, 1H), 1.75-1.68 (m, 1H), 1.59 (d, J = 6.5 Hz, 3H), 1.26 (d, J = 5.4 Hz, 3H), 1.16 (d, J = 5.5 Hz, 3H) ³¹P NMR (162 MHz, CDCl₃) δ 29.27 (s). ¹⁹F NMR (377 MHz, CDCl₃) δ −113.34 (s), −128.43 (s). |
| 223 | 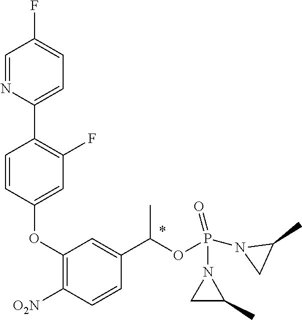 [SMILES: CC(OP(=O)(N1C[C@@H]1C)N1C[C@@H]1C)c1ccc([N+](=O)[O-])c(Oc2ccc(-c3ccc(F)cn3)c(F)c2)c1] | LCMS(ESI): m/z 531.3 [M + H]⁺; ¹H NMR (400 MHz, CDCl₃) δ 8.56 (d, J = 2.8 Hz, 1H), 8.07-7.95 (m, 2H), 7.81-7.76 (m, 1H), 7.61-7.46 (m, 1H), 7.31 (d, J = 8.5 Hz, 1H), 7.20 (s, 1H), 6.93-6.90 (m, 1H), 6.86-6.81 (m, 1H), 5.68-5.58 (m, 1H), 2.60-2.49 (m, 1H), 2.48-2.39 (m, 1H), 2.34-2.28 (m, 1H), 2.21-2.16 (m, 1H), 1.82-1.70 (m, 2H), 1.59 (d, J = 6.5 Hz, 3H), 1.26 (d, J = 5.0 Hz, 3H), 1.16 (d, J = 5.3 Hz, 3H). ³¹P NMR (162 MHz, CDCl₃) δ 29.26 (s). ¹⁹F NMR (377 MHz, CDCl₃) δ −113.33 (s), −128.43 (s). |
| 224 | 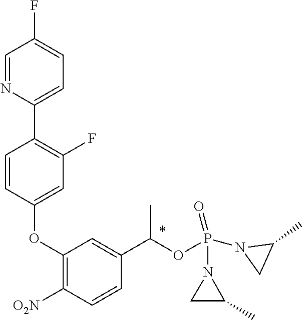 [SMILES: CC(OP(=O)(N1C[C@H]1C)N1C[C@H]1C)c1ccc([N+](=O)[O-])c(Oc2ccc(-c3ccc(F)cn3)c(F)c2)c1] | LCMS(ESI): m/z 531.3 [M + H]⁺; ¹H NMR (400 MHz, CDCl₃) δ 8.56 (d, J = 2.9 Hz, 1H), 8.08-7.93 (m, 2H), 7.82-7.75 (m, 1H), 7.52-7.44 (m, 1H), 7.33 (dd, J = 8.5, 1.5 Hz, 1H), 7.20 (d, J = 1.5 Hz, 1H), 6.91 (dd, J = 8.7, 2.3 Hz, 1H), 6.83 (dd, J = 11.9, 2.4 Hz, 1H), 5.70-5.55 (m, 1H), 2.58-2.39 (m, 2H), 2.35-2.29 (m, 1H), 2.25-2.19 (m, 1H), 1.91-1.87 (m, 1H), 1.72-1.68 (m, 1H), 1.59 (d, J = 6.3 Hz, 3H), 1.20 (dd, J = 5.4, 0.9 Hz, 3H), 1.14 (d, J = 5.3 Hz, 3H). ³¹P NMR (162 MHz, CDCl₃) δ 29.18 (s). ¹⁹F NMR (377 MHz, CDCl₃) δ −113.37 (s), −128.44 (s). |
| 225 | 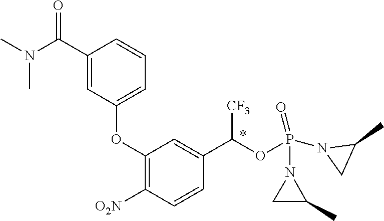 | LCMS(ESI): m/z 543.2 [M + H]⁺; ¹H NMR (400 MHz, CDCl₃) δ 8.03 (d, J = 7.8 Hz, 1H), 7.50-7.36 (m, 2H), 7.29-7.18 (m, 2H), 7.17-7.01 (m, 2H), 5.85- 5.58 (m, 1H), 3.11 (s, 3H), 3.00 (s, 3H), 2.69-2.55 (m, 1H), 2.51-2.33 (m, 2H), 2.25-2.13 (m, 1H), 2.02-1.73 (m, 2H), 1.35-1.05 (m, 6H). ³¹P NMR (162 MHz, CDCl₃) δ 30.99 (d, J = 8.8 Hz). ¹⁹F NMR (376 MHz, CDCl₃) δ −76.81 (d, J = 19.4 Hz). |
| 226 | 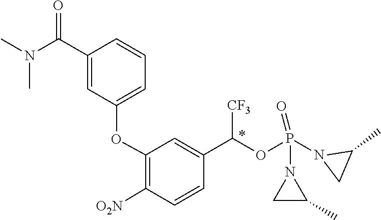 | LCMS(ESI): m/z 543.1 [M + H]⁺; ¹H NMR (400 MHz, CDCl₃) δ 8.01 (d, J = 8.4 Hz, 1H), 7.49-7.33 (m, 2H), 7.26-7.20 (m, 2H), 7.13-7.03 (m, 2H), 5.76-5.63 (m, 1H), 3.09 (s, 3H), 2.98 (s, 3H), 2.65-2.50 (m, 1H), 2.49-2.40 (m, 1H), 2.40-2.31 (m, 1H), 2.22-2.09 (m, 1H), 1.97-1.80 (m, 1H), 1.77-1.68 (m, 1H), 1.31-1.20 (m, 3H), 1.16-1.06 (m, 3H). ³¹P NMR (162 MHz, CDCl₃) ) δ 30.99 (d, J = 8.2 Hz). ¹⁹F NMR (376 MHz, CDCl₃) δ −76.81 (d, J = 19.5 Hz). |
| 227 | 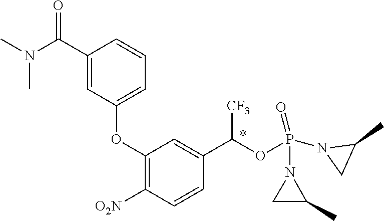 | LCMS(EST): m/z 543.2 [M + H]⁺; ¹H NMR (400 MHz, CDCl₃) δ 7.94 (d, J = 8.4 Hz, 1H), 7.39-7.29 (m, 2H), 7.20-7.13 (m, 2H), 7.04-6.96 (m, 2H), 5.70-5.59 (m, 1H), 3.02 (s, 3H), 2.90 (s, 3H), 2.58-2.46 (m, 1H), 2.42-2.23 (m, 2H), 2.14-2.04 (m, 1H), 1.90-1.65 (m, 2H), 1.21-1.13 (m, 3H), 1.09-1.01 (m, 3H). ³¹P NMR (162 MHz, CDCl₃) δ 30.98 (d, J = 8.4 Hz). ¹⁹F NMR (376 MHz, CDCl₃) δ −76.81 (d, J = 19.4 Hz), |
| 228 | 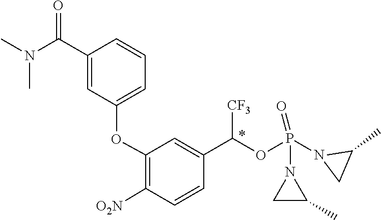 | LCMS(ESI): m/z 543.1 [M + H]⁺; ¹H NMR (400 MHz, CDCl₃) δ 8.09-7.89 (m, 1H), 7.50-7.33 (m, 2H), 7.27-7.18 (m, 2H), 7.12-7.00 (m, 2H), 5.81-5.61 (m, 1H), 3.09 (s, 3H), 2.98 (s, 3H), 2.68-2.51 (m, 1H), 2.51-2.29 (m, 2H), 2.21-2.11 (m, 1H), 1.98-1.80 (m, 1H), 1.77-1.69 (m, 1H), 1.31-1.18 (m, 3H), 1.17-1.07 (m, 3H). ³¹P NMR (162 MHz, CDCl₃) δ 30.98 (d, J = 8.1 Hz). ¹⁹F NMR (376 MHz, CDCl₃) δ −76.81 (d, J = 19.5 Hz) |

TABLE 7
Characterization of compounds (see e.g., Illustration 33)

| Com- | | |
|---|---|---|
| pound | | |
| No. | Structure | LCMS; ¹H NMR (ppm); Retention time |
| 229 | 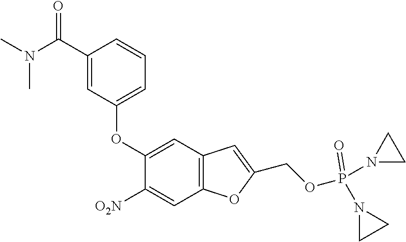 [SMILES: CN(C)C(=O)c1cccc(Oc2cc3cc(COP(=O)(N4CC4)N4CC4)oc3cc2[N+](=O)[O-])c1] | LCMS(ESI): m/z 487.00 [M + H]⁺; ¹H NMR (400 MHz, DMSO-d₆) δ 8.54 (d, J = 0.9 Hz, 1H), 7.63 (s, 1H), 7.45 (t, J = 7.9 Hz, 1H), 7.18-7.08 (m, 3H), 6.99 (dd, J = 2.7, 1.4 Hz, 1H), 5.26 (d, J = 8.2 Hz, 2H), 2.94 (s, 3H), 2.91 (s, 3H), 2.15-2.08 (m, 8H). ³¹P NMR (162 MHz, DMSO-d₆) δ 30.10 (s). |
| 230 | 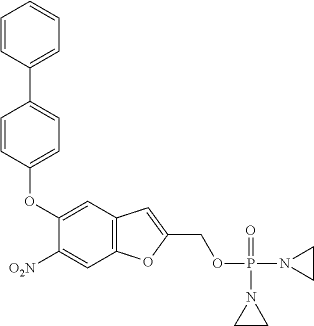 [SMILES: O=[N+]([O-])c1cc2oc(COP(=O)(N3CC3)N3CC3)cc2cc1Oc1ccc(-c2ccccc2)cc1] | LCMS(ESI): m/z 492.15 [M + H]⁺; ¹H NMR (400 MHz, DMSO-d₆) δ 8.55 (d, J = 0.9 Hz, 1H), 7.74-7.58 (m, 5H), 7.47 (t, J = 7.5 Hz, 2H), 7.36 (t, J = 7.3 Hz, 1H), 7.20-7.08 (m, 3H), 5.27 (d, J = 8.3 Hz, 2H), 2.23-2.00 (m, 8H). ³¹P NMR (162 MHz, DMSO-d₆) δ 31.12 (s). |
| 231 | 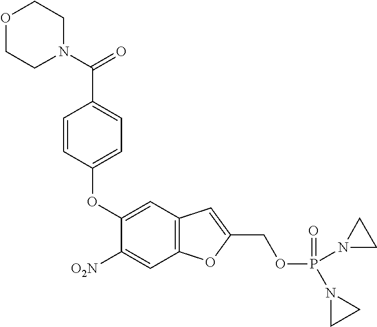 [SMILES: O=C(c1ccc(Oc2cc3cc(COP(=O)(N4CC4)N4CC4)oc3cc2[N+](=O)[O-])cc1)N1CCOCC1] | LCMS(ESI): m/z 528.95 [M + H]⁺; ¹H NMR (400 MHz, DMSO-d₆) δ 8.56 (d, J = 0.9 Hz, 1H), 7.66 (s, 1H), 7.48-7.40 (m, 2H), 7.14 (s, 1H), 7.10- 7.02 (m, 2H), 5.27 (d, J = 8.3 Hz, 2H), 3.69-3.41 (m, 8H), 2.18-2.05 (m, 8H). ³¹P NMR (162 MHz, DMSO-d₆) δ 31.12 (s). |
| 232 | 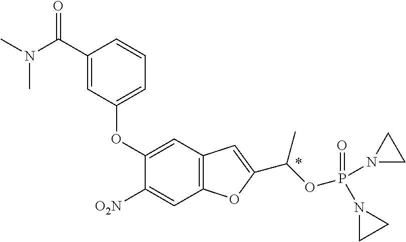 [SMILES: CC(OP(=O)(N1CC1)N1CC1)c1cc2cc(Oc3cccc(C(=O)N(C)C)c3)c([N+](=O)[O-])cc2o1] | LCMS(ESI): m/z 500.95 [M + H]⁺; ¹H NMR (400 MHz, DMSO-d₆) δ 8.53 (d, J = 1.0 Hz, 1H), 7.61 (d, J = 1.4 Hz, 1H), 7.45 (t, J = 7.9 Hz, 1H), 7.19-.13 (m, 1H), 7.13-7.07 (m, 2H), 7.01-6.94 (m, 1H), 5.78-5.69 (m, 1H), 2.93 (s, 3H), 2.87 (s, 3H), 2.17-2.03 (m, 8H), 1.67 (d, J = 6.6 Hz, 3H). ³¹P NMR (162 MHz, DMSO-d) δ 30.48 (s). ee: >99% Retention time: 2.664 min; Column: Column 2-70A1-30Ba-3.6 min- 1.67 v · M, Mobile Phase: (MtBE:Hex = 1:1) (0.1% DEA):MeOH=70:30 Flow rate: 1.67 ml/min, Temperature:Ambient. |
| 233 | 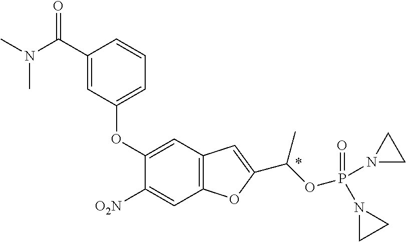 [SMILES: CC(OP(=O)(N1CC1)N1CC1)c1cc2cc(Oc3cccc(C(=O)N(C)C)c3)c([N+](=O)[O-])cc2o1] | LCMS(ESI): m/z 501.00 [M + H]⁺; ¹H NMR (400 MHz, DMSO-d₆) δ 8.53 (s, 1H), 7.61 (s, 1H), 7.49-7.40 (m, 1H), 7.19-7.11 (m, 1H), 7.13-7.06 (m, 2H), 7.01-6.94 (m, 1H), 5.78- 5.67 (m, 1H), 2.94 (s, 3H), 2.87 (s, 3H), 2.18-2.01 (m, 8H), 1.67 (d, J = 6.6 Hz, 3H). ³¹P NMR (162 MHz, DMSO-d₆) δ 30.48 (s). ee: >99% Retention time: 3.458 min; Column: Column 2-70A1-30Ba-3.6 min- 1.67 v · M, Mobile Phase: (MtBE:Hex = 1:1)(0.1% DEA):MeOH = 70:30 Flow rate: 1.67 ml/min, Temperature:Ambient. |

TABLE 8
Characterization of compounds (see Illustration 14 & 16)

| Com- | | |
|---|---|---|
| pound | | |
| No. | Structure | LCMS; ¹H NMR (ppm); Retention time |
| 234 | 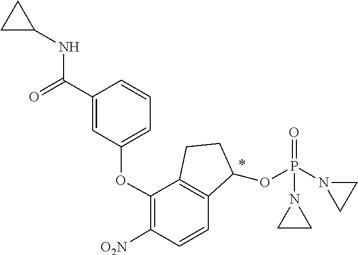 [SMILES: O=C(NC1CC1)c1cccc(Oc2c([N+](=O)[O-])ccc3c2CCC3OP(=O)(N2CC2)N2CC2)c1] | LCMS(ESI): m/z 485.20 [M + H]⁺; ¹H NMR (400 MHz, DMSO-d₆) δ 8.50 (d, J = 4.1 Hz, 1H), 8.05 (d, J = 8.2 Hz, 1H), 7.60-7.53 (m, 2H), 7.43 (t, J = 8.0 Hz, 1H), 7.38-7.33 (m, 1H), 7.03 (dd, J = 8.1, 2.7 Hz, 1H), 5.97-5.91 (m, 1H), 2.84- 2.77 (m, 1H), 2.73-2.64 (m, 1H), 2.57-2.51 (m, 1H), 2.17-2.08 (m, 9H), 0.71-0.65 (m, 2H), 0.58-0.53 (m, 2H). ee: >94% Retention time: 4.180 min; Column: Lux Cellulose-3, 0.46 × 10 cm, 3.0 um; Co-solvent: MeOH (0.1% DEA); Gradient: 5% to 20% in 4.0 min, hold 2.0 min at 20%; Back pressure (bar): 110; Flow rate: 2.0 mL/min; Temperature: 35° C. |
| 235 | 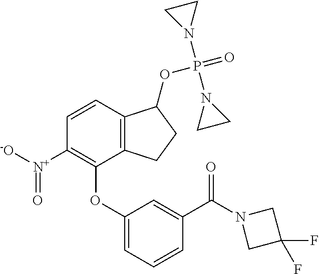 | LCMS(ESI): m/z 521.1 [M + H]⁺; ¹H NMR (400 MHz, CDCl₃) δ 7.92 (d, J = 8.2 Hz, 1H), 7.56 (d, J = 8.2 Hz, 1H), 7.42-7.37 (m, 1H), 7.31 (d, J = 7.6 Hz, 1H), 7.12 (s, 1H), 7.06-7.02 (m, 1H), 6.05-5.98 (m, 1H), 4.56-4.42 (m, 4H), 2.96-2.80 (m, 1H), 2.69-2.52 (m, 2H), 2.30-2.16 (m, 9H). |
| 236 | 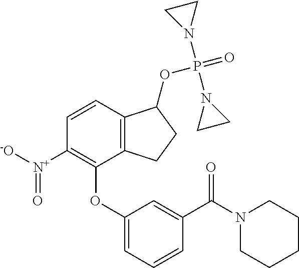 | LCMS(ESI): m/z 513.2 [M + H]⁺; ¹H NMR (400 MHz, CDCl₃) δ7.89 (d, J = 8.2 Hz, 1H), 7.53 (d, J = 8.2 Hz, 1H), 7.34 (t, J = 7.9 Hz, 1H), 7.09 (d, J = 7.6 Hz, 1H), 6.93 (dd, J = 8.2, 2.1 Hz, 1H), 6.84 (s, 1H), 6.04-5.96 (m, 1H), 3.74-3.58 (m, 2H), 3.38-3.24 (m, 2H), 2.93-2.83 (m, 1H), 2.68-2.51 (m, 2H), 2.32-2.08 (m, 9H), 1.73-1.64 (m, 4H), 1.53-1.43 (m, 2H). |
| 237 | 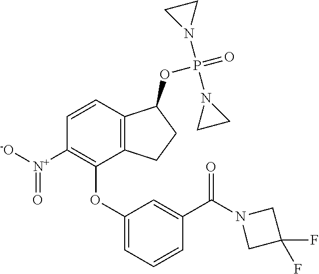 | LCMS(ESI): m/z 521.1 [M + H]⁺; ¹H NMR (400 MHz, CDCl₃) δ7.92 (d, J = 8.2 Hz, 1H), 7.56 (d, J = 8.2 Hz, 1H), 7.43-7.36 (m, 1H), 7.33-7.28 (m, 1H), 7.13-7.10 (m, 1H), 7.07-7.01 (m, 1H), 6.06-5.97 (m, 1H), 4.50 (t, J = 12.0 Hz, 4H), 2.92- 2.81 (m, 1H), 2.67-2.52 (m, 2H), 2.28-2.15 (m, 9H). ee: >99% Retention time: 2.789 min (Method 8). |
| 238 | 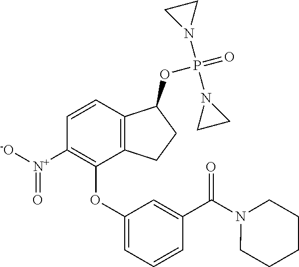 | LCMS(ESI): m/z 513.4 [M + H]⁺; ¹H NMR (400 MHz, CDCl₃) δ7.87 (d, J = 8.2 Hz, 1H), 7.51 (d, J = 8.2 Hz, 1H), 7.32 (t, J = 7.9 Hz, 1H), 7.10-7.05 (m, 1H), 6.95-6.88 (m, 1H), 6.84-6.80 (m, 1H), 6.02-5.95 (m, 1H), 3.72-3.57 (m, 2H), 3.35-3.20 (m, 2H), 2.91-2.79 (m, 1H), 2.65-2.48 (m, 2H), 2.26- 2.16 (m, 9H), 1.70- 1.56 (m, 4H), 1.53-1.41 (m, 2H). ee: >99% Retention time: 2.513 min (Method 9). |
| 239 | 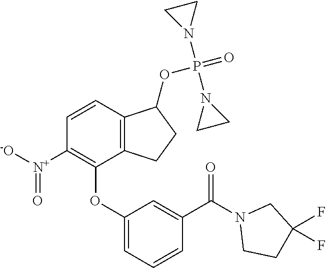 | LCMS(ESI): m/z 535.1 [M + H]⁺; ¹H NMR (400 MHz, CDCl₃) δ 7.90 (d, J = 8.2 Hz, 1H), 7.55 (d, J = 8.2 Hz, 1H), 7.38 (t, J = 8.1 Hz, 1H), 7.20 (s, 1H), 6.98 (d, J = 7.2 Hz, 2H), 6.04-5.98 (m, 1H), 4.02-3.57 (m, 5H), 2.94-2.82 (m, 1H), 2.69-2.52 (m, 2H), 2.50-2.32 (m, 2H) 2.31-2.15 (m, 9H). |
| 240 | 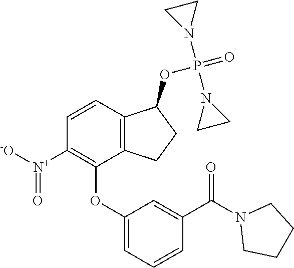 | LCMS(ESI): m/z 499.2 [M + H]⁺; ¹H NMR (400 MHz, CDCl₃) δ 7.89 (d, J = 8.2 Hz, 1H), 7.53 (d, J = 8.2 Hz, 1H), 7.34 (t, J = 7.8 Hz, 1H), 7.21 (d, J = 7.6 Hz, 1H), 6.99-6.92 (m, 2H), 6.05-5.95 (m, 1H), 3.60 (t, J = 6.8 Hz, 2H), 3.37 (t, J = 6.5 Hz, 2H), 2.92-2.82 (m, 1H), 2.67-2.51 (m, 2H), 2.29-2.18 (m, 9H), 1.97- 1.85 (m, 4H). ee: >99% Retention time: 4.289 min (Method 13). |
| 241 | 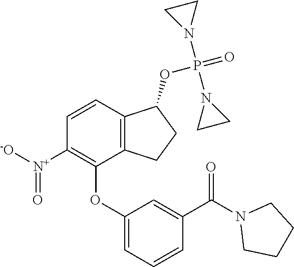 | LCMS(ESI): m/z 499.2 [M + H]⁺; ¹H NMR (400 MHz, CDCl₃) δ 7.89 (d, J = 8.2 Hz, 1H), 7.53 (d, J = 8.2 Hz, 1H), 7.35 (t, J = 7.8 Hz, 1H), 7.21 (d, J = 7.6 Hz, 1H), 6.99-6.93 (m, 2H), 6.00 (dd, J = 12.3, 7.1 Hz, 1H), 3.60 (t, J = 6.8 Hz, 2H), 3.37 (t, J = 6.5 Hz, 2H), 2.93-2.81 (m, 1H), 2.67-2.51 (m, 2H), 2.29-2.18 (m, 9H), 1.97-1.85 (m, 4H). ee: >99% Retention time: 4.654 min (Method 13). |
| 242 | 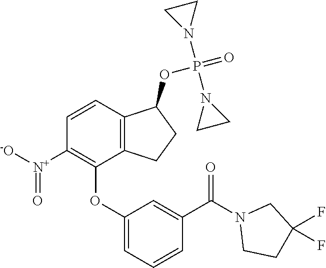 | LCMS(ESI): m/z 535.1 [M + H]⁺; ¹H NMR (400 MHz, CDCl₃) δ 7.83 (d, J = 8.2 Hz, 1H), 7.48 (d, J = 8.2 Hz, 1H), 7.31 (t, J = 8.3 Hz, 1H), 7.17-7.09 (m, 1H), 6.95-6.87 (m, 2H), 5.98-5.89 (m, 1H), 3.93-3.54 (m, 4H), 2.86-2.75 (m, 1H), 2.61-2.45 (m, 2H), 2.40-2.24 (m, 2H), 2.22-2.09 (m, 9H). ee: >99% Retention time: 5.742 min (Method 14). |
| 243 | 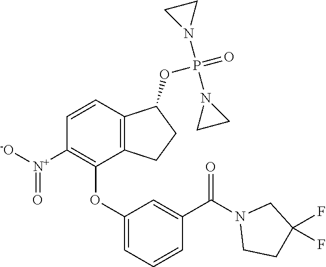 | LCMS(ESI): m/z 535.1 [M + H]⁺; ¹H NMR (400 MHz, CDCl₃) δ 7.83 (d, J = 8.2 Hz, 1H), 7.48 (d, J = 8.2 Hz, 1H), 7.31 (t, J = 8.2 Hz, 1H), 7.16-7.08 (m, 1H), 6.94-6.88 (m, 2H), 5.98-5.90 (m, 1H), 3.95-3.53 (m, 4H), 2.87-2.74 (m, 1H), 2.62-2.45 (m, 2H), 2.41-2.24 (m, 2H), 2.22-2.09 (m, 9H). ee: 97% Retention time: 6.231 min (Method 14). |
| 244 | 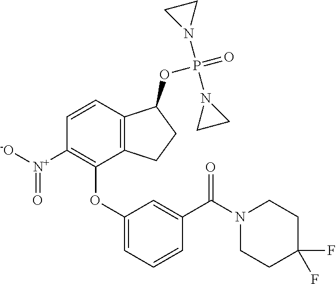 | LCMS(ESI): m/z 549.2 [M + H]⁺; ¹H NMR (400 MHz, CDCl₃) δ 7.88 (d, J = 8.2 Hz, 1H), 7.54 (d, J = 8.2 Hz, 1H), 7.36 (t, J = 7.9 Hz, 1H), 7.10 (d, J = 7.6 Hz, 1H), 6.99-6.93 (m, 1H), 6.86 (s, 1H), 6.05-5.96 (m, 1H), 3.95-3.40 (m, 4H), 2.95-2.84 (m, 1H), 2.69-2.52 (m, 2H), 2.29-2.17 (m, 9H), 2.12-1.86 (m, 4H). ee: >99% Retention time: 2.527 min (Method 13). |
| 245 | 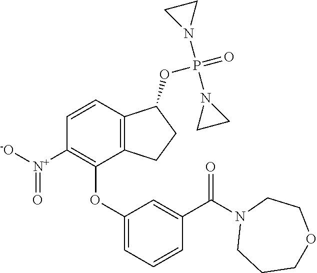 | LCMS(ESI): m/z 529.2 [M + H]⁺; ¹H NMR (400 MHz, CDCl₃) δ 7.89 (d, J = 8.2 Hz, 1H), 7.54 (d, J = 8.2 Hz, 1H), 7.39-7.32 (m, 1H), 7.10 (d, J = 7.5 Hz, 1H), 6.98-6.90 (m, 1H), 6.86 (s, 1H), 6.07-5.86 (m, 1H), 3.87-3.78 (m, 4H), 3.77-3.73 (m, 1H), 3.66-3.61 (m, 1H), 3.51-3.44 (m, 2H), 2.96-2.83 (m, 1H), 2.69- 2.51 (m, 2H), 2.31-2.15 (m, 9H), 2.08-1.97 (m, 1H), 1.85-1.77 (m, 1H). ee: 97% Retention time: 4.449 min (Method 13). |
| 246 | 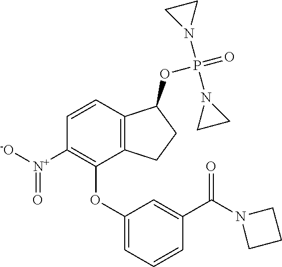 | LCMS(ESI): m/z 485.1 [M + H]⁺; ¹H NMR (400 MHz, CDCl₃) δ 7.90 (d, J = 8.2 Hz, 1H), 7.53 (d, J = 8.2 Hz, 1H), 7.38-7.29 (m, 2H), 7.09-7.05 (m, 1H), 7.01 (d, J = 6.6 Hz, 1H), 6.05-5.95 (m, 1H), 4.26-4.15 (m, 4H), 2.90-2.80 (m, 1H), 2.67- 2.49 (m, 2H), 2.37-2.29 (m, 2H), 2.29-2.14 (m, 9H). ee: >99% Retention time: 4.824 min (Method 3). |
| 247 | 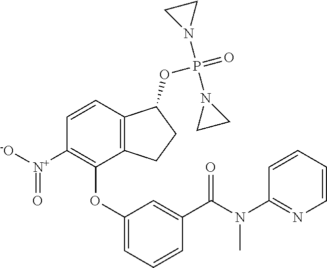 | LCMS(ESI): m/z 536.2 [M + H]⁺; ¹H NMR (400 MHz, CDCl₃) δ 8.38 (d, J = 5.8 Hz, 1H), 7.98 (d, J = 8.2 Hz, 1H), 7.61 (d, J = 8.2 Hz, 1H), 6.70-6.65 (m, 1H), 6.64-6.58 (m, 1H), 6.13-5.95 (m, 1H), 2.97-2.86 (m, 1H), 2.73-2.58 (m, 2H), 2.56 (s, 3H), 2.33-2.17 (m, 9H). ee: >99% Retention time: 3.707 min (Method 13). |
| 248 | 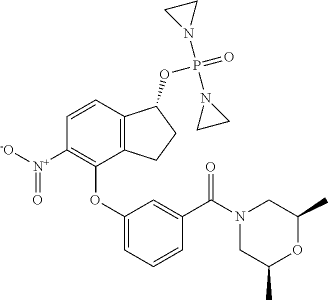 | LCMS(ESI): m/z 543.3 [M + H]⁺; ¹H NMR (400 MHz, CDCl₃) δ 7.89 (d, J = 8.2 Hz, 1H), 7.54 (d, J = 8.2 Hz, 1H), 7.39-7.34 (m, 1H), 7.11-7.08 (m, 1H), 6.98-6.93 (m, 1H), 6.86-6.83 (m, 1H), 6.05-5.97 (m, 1H), 4.58-4.45 (m, 1H), 3.65-3.59 (m, 1H), 3.55-3.44 (m, 2H), 2.94-2.85 (m, 1H), 2.82-2.70 (m, 1H), 2.68- 2.50 (m, 3H), 2.29-2.16 (m, 9H), 1.25-1.17 (m, 3H), 1.16-1.08 (m, 3H). ee: 97% Retention time: 2.792 min (Method 13). |
| 249 | 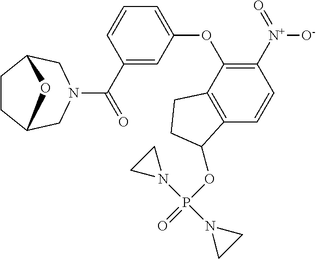 [SMILES: O=C(c1cccc(Oc2c([N+](=O)[O-])ccc3c2CCC3OP(=O)(N2CC2)N2CC2)c1)N1C[C@H]2CC[C@@H](C1)O2] | LCMS(ESI): m/z 541.3 [M + H]⁺; ¹H NMR (400 MHz, CDCl₃) δ 7.89 (d, J = 8.2 Hz, 1H), 7.54 (d, J = 8.2 Hz, 1H), 7.36 (t, J = 7.9 Hz, 1H), 7.10-7.06 (m, 1H), 6.96-6.91 (m, 1H), 6.85-6.82 (m, 1H), 6.05-5.98 (m, 1H), 4.46-4.23 (m, 3H), 3.44-3.24 (m, 2H), 3.15-3.06 (m, 1H), 2.95-2.83 (m, 1H), 2.69-2.52 (m, 2H), 2.29- 2.17 (m, 9H), 1.98-1.82 (m, 3H), 1.67-1.61 (m, 1H). LCMS(ESI): m/z 541.2 [M + H]⁺; |
| 250 | 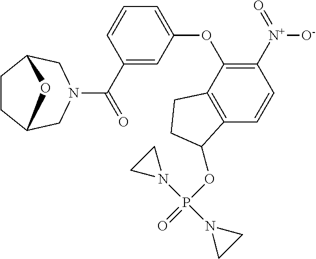 [SMILES: O=C(c1cccc(Oc2c([N+](=O)[O-])ccc3c2CCC3OP(=O)(N2CC2)N2CC2)c1)N1C[C@H]2CC[C@@H](C1)O2] | ¹H NMR (400 MHz, CDCl₃) δ 7.89 (d, J = 8.2 Hz, 1H), 7.55 (d, J = 8.2 Hz, 1H), 7.36 (t, J = 7.9 Hz, 1H), 7.12-7.05 (m, 1H), 6.94 (dd, J = 8.2, 2.1 Hz, 1H), 6.86-6.81 (m, 1H), 6.05-5.97 (m, 1H), 4.50-4.19 (m, 3H), 3.46-3.22 (m, 2H), 3.16-3.04 (m, 1H), 2.96-2.81 (m, 1H), 2.71-2.52 (m, 2H), 2.32-2.16 (m, 9H), 2.02-1.82 (m, 3H), 1.69-1.56 (m, 1H). ee: 97% Retention time: 8.161 min (Method 14). |
| 251 | 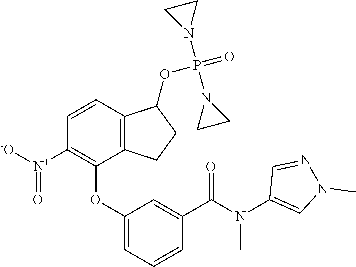 | LCMS(ESI): m/z 539.2 [M + H]⁺; ¹H NMR (400 MHz, CDCl₃) δ 7.90 (d, J = 8.2 Hz, 1H), 7.53 (d, J = 8.2 Hz, 1H), 7.43-7.30 (m, 1H), 7.25-6.67 (m, 5H), 6.05-5.96 (m, 1H), 3.94-3.71 (m, 3H), 3.38-3.26 (m, 3H), 2.93-2.77 (m, 1H), 2.64-2.51 (m, 2H), 2.30-2.17 (m, 9H). |
| 252 | 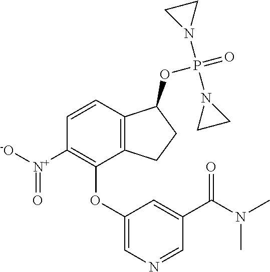 | LCMS(ESI): m/z 474.2 [M + H]⁺; ¹H NMR (400 MHz, CDCl₃) δ 8.42 (d, J = 2.9 Hz, 1H), 8.37 (d, J = 1.5 Hz, 1H), 8.09 (d, J = 8.2 Hz, 1H), 7.59 (d, J = 8.3 Hz, 1H), 7.41-7.31 (m, 1H), 5.99-5.89 (m, 1H), 2.96 (s, 3H), 2.86 (s, 3H), 2.81-2.73 (m, 1H), 2.64-2.53 (m, 2H), 2.20-2.05 (m, 9H). ee: >99% Retention time: 1.224 min (Method 7). |
| 253 | 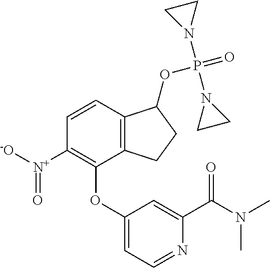 | LCMS(ESI): m/z 474.1 [M + H]⁺; ¹H NMR (400 MHz, CDCl₃) δ 8.48 (d, J = 5.7 Hz, 1H), 7.97 (d, J = 8.0 Hz, 1H), 7.62 (d, J = 8.2 Hz, 1H), 7.05 (d, J = 2.3 Hz, 1H), 6.90-6.84 (m, 1H), 6.08-6.00 (m, 1H), 3.10 (s, 3H), 3.08 (s, 3H), 3.00-2.91 (m, 1H), 2.75-2.58 (m, 2H), 2.35-2.14 (m, 9H). |
| 254 | 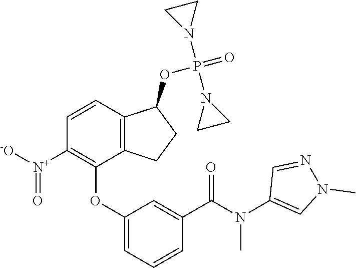 | LCMS(ESI): m/z 539.2 [M + H]⁺; ¹H NMR (400 MHz, CDCl₃) δ 7.90 (d, J = 8.2 Hz, 1H), 7.53 (d, J = 8.2 Hz, 1H), 7.26-7.10 (m, 2H), 7.06 (s, 1H), 6.80 (d, J = 58.5 Hz, 3H), 6.01 (dd, J = 12.1, 7.0 Hz, 1H), 3.94-3.72 (m, 3H), 3.34 (s, 3H), 2.85 (s, 1H), 2.63-2.50 (m, 2H), 2.29-2.18 (m, 9H). ee: >99% Retention time: 4.172 min (Method 31). |
| 255 | 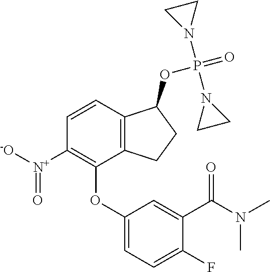 | LCMS(ESI): m/z 491.2 [M + H]⁺; ¹H NMR (400 MHz, CDCl₃) δ 7.88 (d, J = 8.2 Hz, 1H), 7.53 (d, J = 8.1 Hz, 1H), 7.05 (t, J = 8.8 Hz, 1H), 6.96-6.89 (m, 1H), 6.86-6.80 (m, 1H), 6.04-5.96 (m, 1H), 3.09 (s, 3H), 2.93 (s, 3H), 2.92-2.83 (m, 1H), 2.71- 2.53 (m, 2H), 2.31-2.15 (m, 9H). ee: >99% Retention time: 2.619 min (Method 1). |
| 256 | 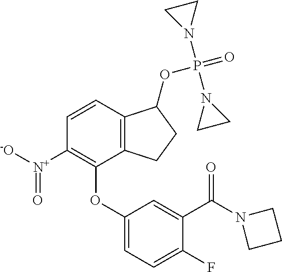 | LCMS(ESI): m/z 503.1[M + H]⁺; ¹H NMR (400 MHz, CDCl₃) δ 7.95-7.83 (m, 1H), 7.53 (d, J = 7.9 Hz, 1H), 7.08-7.01 (m, 1H), 7.00-6.88 (m, 2H), 6.05-5.93 (m, 1H), 4.21- 4.14 (m, 2H), 4.13-4.06 (m, 2H), 2.95-2.82 (m, 1H), 2.68-2.53 (m, 2H), 2.36-2.30 (m, 2H), 2.30-2.12 (m, 9H). |
| 257 | 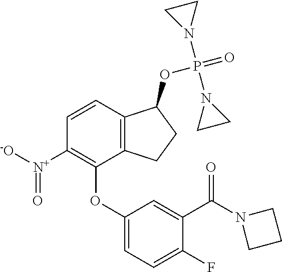 | LCMS(ESI): m/z 503.1 [M + H]⁺; ¹H NMR (400 MHz, CDCl₃) δ7.89 (d, J = 8.2 Hz, 1H), 7.54 (d, J = 8.2 Hz, 1H), 7.08-7.02 (m, 1H), 6.99-6.91 (m, 2H), 6.04-5.94 (m, 1H), 4.22-4.14 (m, 2H), 4.13-4.06 (m, 2H), 2.95-2.81 (m, 1H), 2.68-2.53 (m, 2H), 2.38-2.30 (m, 2H), 2.29-2.17 (m, 9H). ee: >99% Retention time: 5.073 min (Method 35). |
| 258 | 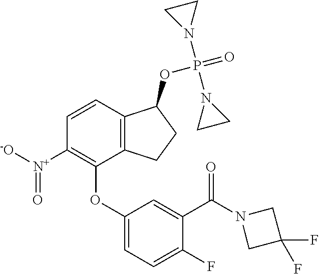 | LCMS(ESI): m/z 539.1 [M + H]⁺; ¹H NMR (400 MHz, CDCl₃) δ 7.91 (d, J = 8.2 Hz, 1H), 7.56 (d, J = 8.2 Hz, 1H), 7.13-7.06 (m, 1H), 7.05-6.99 (m, 2H), 6.07-5.96 (m, 1H), 4.52-4.39 (m, 4H), 2.96-2.83 (m, 1H), 2.69-2.55 (m, 2H), 2.30-2.17 (m, 9H). ee: >99% Retention time: 3.800 min (Method 40). |
| 259 | 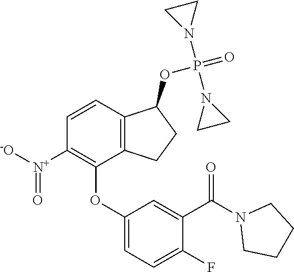 | LCMS(ESI): m/z 517.2 [M + H]⁺; ¹H NMR (400 MHz, CDCl₃) δ 7.88 (d, J = 8.2 Hz, 1H), 7.53 (d, J = 8.2 Hz, 1H), 7.08-7.01 (m, 1H), 6.96-6.89 (m, 1H), 6.87-6.82 (m, 1H), 6.05-5.95 (m, 1H), 3.63-3.57 (m, 2H), 3.34-3.27 (m, 2H), 2.95-2.83 (m, 1H), 2.69-2.53 (m, 2H), 2.32-2.14 (m, 9H), 1.99-1.87 (m, 4H). ee: >99% Retention time: 4.162 min (Method 1). |
| 260 | 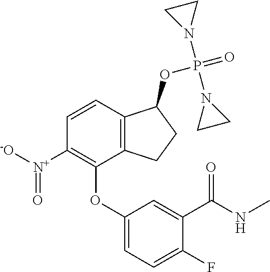 | LCMS(ESI): m/z 477.1 [M + H]⁺; ¹H NMR (400 MHz, CDCl₃) δ 7.91 (d, J = 8.2 Hz, 1H), 7.55-7.47 (m, 2H), 7.13-7.02 (m, 2H), 6.74 (s, 1H), 6.04-5.96 (m, 1H), 3.03-2.97 (m, 3H), 2.91-2.82 (m, 1H), 2.65-2.52 (m, 2H), 2.28-2.17 (m, 9H). ee: 98% Retention time: 3.574 min (Method 46). |
| 261 | 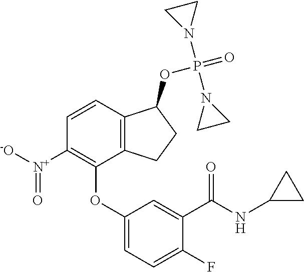 | LCMS(ESI): m/z 503.1 [M + H]⁺; ¹H NMR (400 MHz, CDCl₃) δ 7.91 (d, J = 8.2 Hz, 1H), 7.53 (d, J = 8.2 Hz, 1H), 7.50-7.45 (m, 1H), 7.10-6.99 (m, 2H), 6.87-6.75 (m, 1H), 6.03-5.96 (m, 1H), 2.94-2.79 (m, 2H), 2.68-2.51 (m, 2H), 2.32-2.14 (m, 9H), 0.89-0.84 (m, 2H), 0.65-0.57 (m, 2H). ee: 95% Retention time: 6.796 min (Method 22). |
| 262 | 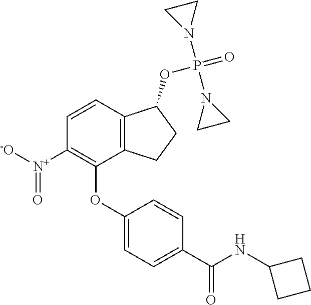 | LCMS(ESI): m/z 499.2 [M + H]⁺; ¹H NMR (400 MHz, CDCl₃) 87.92 (d, J = 8.2 Hz, 1H), 7.72 (d, J = 8.7 Hz, 2H), 7.55 (d, J = 8.2 Hz, 1H), 6.96-6.84 (m, 2H), 6.17 (d, J = 7.4 Hz, 1H), 6.06-5.94 (m, 1H), 4.63-4.51 (m, 1H), 2.95-2.78 (m, 1H), 2.69-2.49 (m, 2H), 2.48-2.36 (m, 2H), 2.33-2.09 (m, 9H), 2.06-1.87 (m, 2H), 1.87-1.73 (m, 2H). ee: >99% Retention time: 8.394 min (Method 5). |
| 263 | 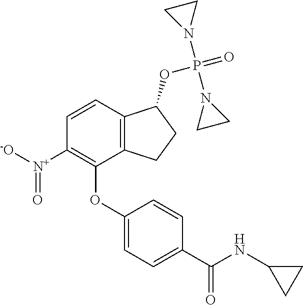 | LCMS(ESI): m/z 485.2 [M + H]⁺; ¹H NMR (400 MHz, CDCl₃) δ 7.91 (d, J = 8.2 Hz, 1H), 7.75-7.68 (m, 2H), 7.54 (d, 1H), 6.91-6.83 (m, 2H), 6.44 (s, 1H), 6.05-5.95 (m, 1H), 2.91-2.79 (m, 2H), 2.64-2.49 (m, 2H), 2.28-2.16 (m, 9H), 0.87-0.80 (m, 2H), 0.65-0.58 (m, 2H). ee: >99% Retention time: 4.970 min (Method 10). |

### Illustration 36. Synthesis of (S)-4-(4-(4-methylpyrimidin-2-yl)phenoxy)-5-nitro-2,3-dihydro-1H-inden-1-yl di(aziridin-1-yl)phosphinate (264)

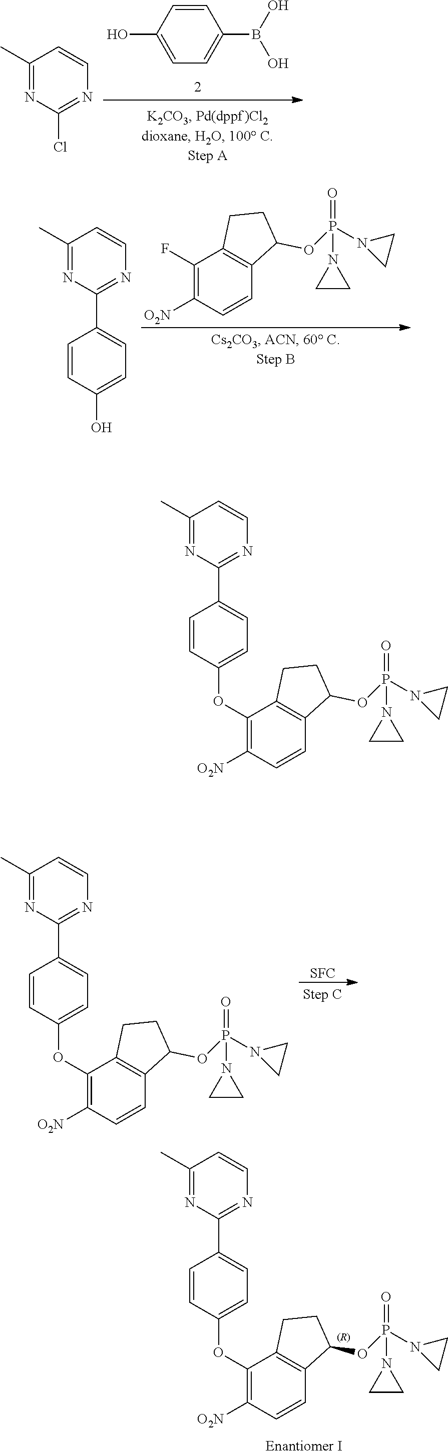

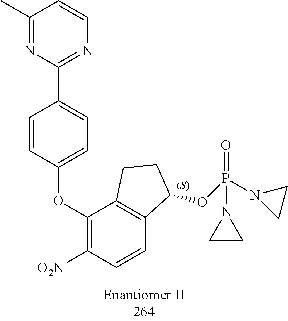

### STEP A: 4-(4-methylpyrimidin-2-yl)phenol

To a solution of 2-chloro-4-methylpyrimidine (500 mg, 3.91 mmol, 1.0 eq.) and (4-hydroxy phenyl) boronic acid (647 mg, 4.69 mmol, 1.2 eq.) and K₂CO₃ (1.62 g, 11.7 mmol, 3.0 eq.) in dioxane/H₂O (15 mL/3 mL) was added Pd(dppf)Cl₂ (285 mg, 0.39 mmol, 0.1 eq.). The resulting mixture was stirred at 100° C. for 12 hrs. After completion, the reaction mixture was concentrated under reduced pressure. The residue was purified by flash column chromatography on silica gel to afford 4-(4-methylpyrimidin-2-yl)phenol (270 mg, 1.45 mmol, 37%) as a yellow solid. LCMS (ESI): m/z 187 [M+H]⁺.

### STEP B: 4-(4-(4-methylpyrimidin-2-yl)phenoxy)-5-nitro-2,3-dihydro-1H-inden-1-yl di(aziridin-1-yl)phosphinate

To a solution of 4-fluoro-5-nitro-2,3-dihydro-1H-inden-1-yl di(aziridin-1-yl)phosphinate (88 mg, 0.27 mmol, 1.0 eq.) and 4-(4-methylpyrimidin-2-yl)phenol (50 mg, 0.27 mmol, 1.0 eq.) in ACN (5 mL) was added Cs₂CO₃ (263 mg, 0.81 mmol, 3.0 eq.). The resulting mixture was stirred at 60° C. for 2 hrs. After completion, the reaction mixture was concentrated under reduced pressure. The residue was purified by prep-HPLC to give 4-(4-(4-methylpyrimidin-2-yl)phenoxy)-5-nitro-2,3-dihydro-1H-inden-1-yl di(aziridin-1-yl)phosphinate (130 mg, 0.26 mmol, 86%) as a white solid. LCMS (ESI): m/z 494 [M+H]⁺.

### STEP C: (R)-4-(4-(4-methylpyrimidin-2-yl)phenoxy)-5-nitro-2,3-dihydro-1H-inden-1-yl di(aziridin-1-yl)phosphinate & (S)-4-(4-(4-methylpyrimidin-2-yl)phenoxy)-5-nitro-2,3-dihydro-1H-inden-1-yl di(aziridin-1-yl)phosphinate (R/S were assigned based on the biological activity)

The racemic mixture, 4-(4-(4-methylpyrimidin-2-yl)phenoxy)-5-nitro-2,3-dihydro-1H-inden-1-yl di(aziridin-1-yl)phosphinate (50 mg, 0.10 mmol) was further separated by Chiral SFC to give:

Enantiomer I, (R)-4-(4-(4-methylpyrimidin-2-yl)phenoxy)-5-nitro-2,3-dihydro-1H-inden-1-yl di(aziridin-1-yl)phosphinate (15 mg, 30%); Retention time: 5.504 min, 97% ee. LC-MS (ESI): m/z 494.1 [M+H]⁺; ¹H NMR (400 MHz, CDCl₃) δ 8.61 (d, J=5.1 Hz, 1H), 8.41 (d, J=8.8 Hz, 2H), 7.91 (d, J=8.2 Hz, 1H), 7.53 (d, J=8.2 Hz, 1H), 7.03 (d, J=5.1 Hz, 1H), 6.96 (d, J=8.8 Hz, 2H), 6.05-5.97 (m, 1H), 2.93-2.82 (m, 1H), 2.66-2.49 (m, 5H), 2.29-2.17 (m, 9H). ³¹P NMR (162 MHz, CDCl₃) δ 29.89 (s).

Enantiomer II, (S)-4-(4-(4-methylpyrimidin-2-yl)phenoxy)-5-nitro-2,3-dihydro-1H-inden-1-yl di(aziridin-1-yl)phosphinate (264, 20 mg, 40%); Retention time: 5.944 min, 98% ee. LC-MS (ESI): m/z 494.1 [M+H]⁺; ¹H NMR (400 MHz, CDCl₃) δ 8.61 (d, J=5.0 Hz, 1H), 8.41 (d, J=8.7 Hz, 2H), 7.91 (d, J=8.2 Hz, 1H), 7.53 (d, J=8.2 Hz, 1H), 7.02 (d, J=5.0 Hz, 1H), 6.96 (d, J=8.7 Hz, 2H), 6.04-5.96 (m, 1H), 2.93-2.82 (m, 1H), 2.66-2.51 (m, 5H), 2.29-2.18 (m, 9H). ³¹P NMR (162 MHz, CDCl₃) δ 29.89 (s).

Analytical method: Column: ChiralPak AD, 100; A4.6 mm I.D., 3 μm, Mobile phase: A for CO₂ and B for EtOH (0.05% DEA), Gradient: 1 min @B 5%, 4 min gradient (5-40%), 2 min @B 40%, then 1.5 min @B 5%, Flow rate: 2.5 mL/min, Back pressure: 100 bar, Column temperature: 35° C.

SFC Method: Instrument: MG II preparative SFC (SFC-14), Column: ChiralPak AD, 250×30 mm I.D., 10 μm, Mobile phase: A for CO₂ and B for Ethanol (0.1% NH₃H₂O), Gradient: B 20%, Flow rate: 70 mL/min, Back pressure: 100 bar, Column temperature: 38° C., Wavelength: 220 nm, Cycle time: ˜6 min.

### Illustration 37. Synthesis of (S)-4-(4-(5-fluoropyrimidin-2-yl)phenoxy)-5-nitro-2,3-dihydro-1H-inden-1-yl di(aziridin-1-yl)phosphinate & (R)-4-(4-(5-fluoropyrimidin-2-yl)phenoxy)-5-nitro-2,3-dihydro-1H-inden-1-yl di(aziridin-1-yl)phosphinate (265 and 266)

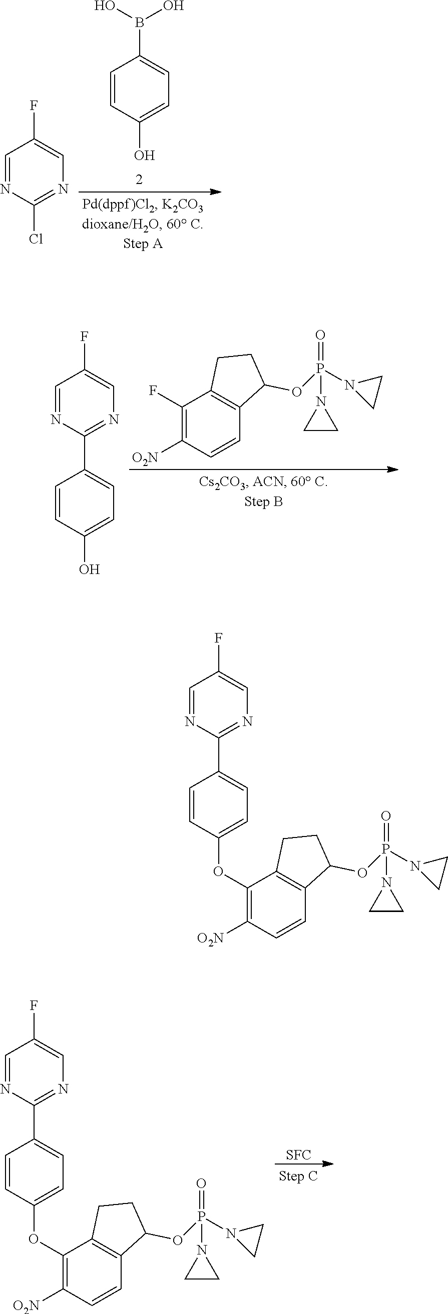

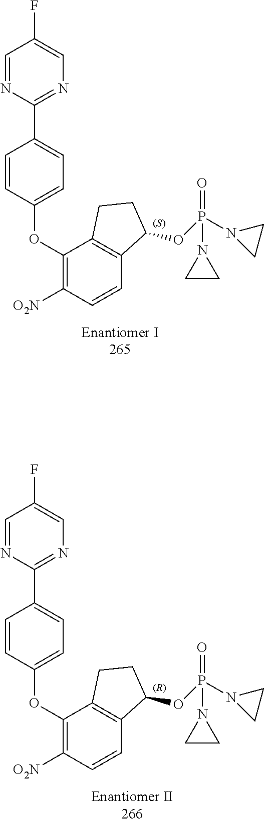

### STEP A: 4-(5-fluoropyrimidin-2-yl)phenol

To a solution of 2-chloro-5-fluoropyrimidine (500 mg, 3.79 mmol, 1.0 eq.) and (4-hydroxy phenyl) boronic acid (785 mg, 5.69 mmol, 1.5 eq.) and K₂CO₃ (1.57 g, 11.4 mmol, 3.0 eq.) in dioxane/H₂O (10 mL/2 mL) was added Pd(dppf)Cl₂ (278 mg, 0.38 mmol, 0.1 eq.). The resulting mixture was stirred at 40° C. for 18 hrs. After completion, the reaction mixture was concentrated under reduced pressure. The residue was purified by flash column chromatography on silica gel to afford 4-(5-fluoropyrimidin-2-yl)phenol (630 mg, 3.32 mmol, 87%) as a yellow solid. LCMS (ESI): m/z 191 [M+H]⁺.

### STEP B: 4-(4-(5-fluoropyrimidin-2-yl)phenoxy)-5-nitro-2,3-dihydro-1H-inden-1-yl di(aziridin-1-yl)phosphinate

To a solution of 4-fluoro-5-nitro-2,3-dihydro-1H-inden-1-yl di(aziridin-1-yl)phosphinate (100 mg, 0.31 mmol, 1.0 eq.) and 4-(5-fluoropyrimidin-2-yl)phenol (64 mg, 0.34 μmol, 1.1 eq.) in ACN (5 mL) was added Cs₂CO₃ (298 mg, 0.92 mmol, 3.0 eq.). The resulting mixture was stirred at 60° C. for 2 hrs. After completion, the reaction mixture was concentrated under reduced pressure. The residue was purified by prep-HPLC to give 4-(4-(5-fluoropyrimidin-2-yl)phenoxy)-5-nitro-2,3-dihydro-1H-inden-1-yl di(aziridin-1-yl)phosphinate (130 mg, 0.26 mmol, 86%) as a white solid. LCMS (ESI): m/z 498 [M+H]⁺.

### STEP C: (S)-4-(4-(5-fluoropyrimidin-2-yl)phenoxy)-5-nitro-2,3-dihydro-1H-inden-1-yl di(aziridin-1-yl)phosphinate & (R)-4-(4-(5-fluoropyrimidin-2-yl)phenoxy)-5-nitro-2,3-dihydro-1H-inden-1-yl di(aziridin-1-yl)phosphinate

The racemic mixture 4-(4-(5-fluoropyrimidin-2-yl)phenoxy)-5-nitro-2,3-dihydro-1H-inden-1-yl di(aziridin-1-yl)phosphinate (140 mg, 0.28 mmol) was further separated by Chiral SFC to give:

Enantiomer 1, (S)-4-(4-(5-fluoropyrimidin-2-yl)phenoxy)-5-nitro-2,3-dihydro-1H-inden-1-yl di(aziridin-1-yl)phosphinate (265, 60 mg, 43%); Retention time: 3.251 min, >99% ee. LC-MS (ESI): m/z 498.1 [M+H]⁺; ¹H NMR (400 MHz, CDCl₃) δ 8.62 (s, 2H), 8.35 (d, J=8.7 Hz, 2H), 7.92 (d, J=8.2 Hz, 1H), 7.54 (d, J=8.2 Hz, 1H), 6.96 (d, J=8.7 Hz, 2H), 6.06-5.96 (m, 1H), 2.95-2.83 (m, 1H), 2.68-2.52 (m, 2H), 2.29-2.16 (m, 9H). 19F NMR (376 MHz, CDCl₃) δ −141.19 (s). ³¹P NMR (162 MHz, CDCl₃) δ 29.95 (s).

Enantiomer 2, (R)-4-(4-(5-fluoropyrimidin-2-yl)phenoxy)-5-nitro-2,3-dihydro-1H-inden-1-yl di(aziridin-1-yl)phosphinate (266, 60 mg, 43%); Retention time: 4.437 min, >99% ee. LC-MS (ESI): m/z 498.1 [M+H]⁺; ¹H NMR (400 MHz, CDCl₃) δ 8.62 (s, 2H), 8.35 (d, J=8.8 Hz, 2H), 7.92 (d, J=8.2 Hz, 1H), 7.54 (d, J=8.2 Hz, 1H), 6.96 (d, J=8.8 Hz, 2H), 6.05-5.98 (m, 1H), 2.94-2.84 (m, 1H), 2.68-2.52 (m, 2H), 2.28-2.19 (m, 9H). ¹⁹F NMR (376 MHz, CDCl₃) δ −141.19 (s). ³¹P NMR (162 MHz, CDCl₃) δ 29.95 (s).

Analytical method: Column: ChiralPak IH, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

SFC Method: Instrument: IMADZU PREP SOLUTION SFC, Column: ChiralPak IH, 250×21.2 mm I.D., 5 μm, Mobile phase: A for CO₂ and B for MEOH+0.1% NH₃H₂O, Gradient: B 20%, Flow rate: 40 mL/min, Back pressure: 100 bar, Column temperature: 35° C., Wavelength: 220 nm, Cycle-time: 4.5 min.

Illustration 38. Synthesis of (R)-4-fluoro-5-nitro-2,3-dihydro-1H-inden-1-yl di(aziridin-1-yl)phosphinate (Int.7)

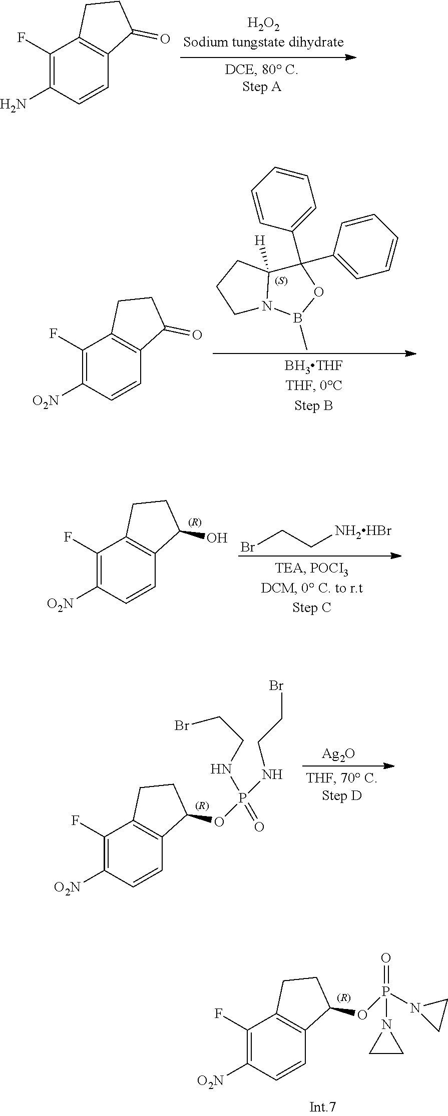

### STEP A: 4-fluoro-5-nitro-2,3-dihydro-1H-inden-1-one

To a solution of 5-amino-4-fluoro-2,3-dihydro-1H-inden-1-one (5 g, 30.3 mmol, 1.0 eq.) and Sodium tungstate dihydrate (3 g, 10.4 mmol, 0.3 eq.) in DCE (100 mL) was added H₂O₂ (50 mL, 30 wt %) at 25° C. The solution was heated to 80° C. and stirred at 80° C. for 6 hrs. After completion, the reaction mixture was diluted with H₂O (300 mL) and extracted with DCM (300 mL×3). The organic layers were combined and washed with brine (100 mL), dried over anhydrous Na₂SO₄, and concentrated under reduced pressure. The residue was purified by flash column chromatography on silica gel to afford 4-fluoro-5-nitro-2,3-dihydro-1H-inden-1-one (1.18 g, 6.05 mmol, 20%) as a yellow solid. LCMS (ESI): m/z 196 [M+H]⁺.

### STEP B: (R)-4-fluoro-5-nitro-2,3-dihydro-1H-inden-1-ol

To a solution of (S)-1-methyl-3,3-diphenyltetrahydro-1H,3H-pyrrolo[1,2-c][1,3,2]oxazaborole (2.98 g, 10.8 mmol, 0.6 eq.) in THF (80 mL) was added BH₃·THF (27 mL, 1 N, 26.9 mmol, 1.5 eq.) at 0° C. under nitrogen. The solution was stirred at 0° C. for 30 min. A solution of 4-fluoro-5-nitro-2,3-dihydro-1H-inden-1-one (3.5 g, 17.9 mmol, 1.0 eq.) in THF (80 mL) was added slowly to the above mixture at 0° C. After addition, the reaction mixture was stirred at 0° C. for 2 hrs. MeOH (80 mL) was added into the reaction mixture at 0° C., and the solution was stirred for 30 min. The mixture was concentrated under reduced pressure. The residue was purified by flash column chromatography on silica gel to give (R)-4-fluoro-5-nitro-2,3-dihydro-1H-inden-1-ol (3.3 g, 16.7 mmol, 93%) as a yellow solid, which was confirmed by XRPD. LCMS (ESI): m/z 198 [M+H]⁺. Retention time: 1.289 min, 93% ee. Analytical method: Column: ChiralPak C-IG, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

### STEP C: (1R)-4-fluoro-5-nitro-2,3-dihydro-1H-indenyl P,P-bis[(2-bromoethyl)amino]phosphinate

To a solution of POCl₃ (463 mg, 3.05 mmol, 1.5 eq.) in DCM (20 mL) was added TEA (615 mg, 6.09 mmol, 3.0 eq.) at 0° C. The resulting mixture was stirred at 0° C. for 15 min. (R)-4-fluoro-5-nitro-2,3-dihydro-1H-inden-1-ol (400 mg, 2.03 mmol, 1.0 eq.) was added into the above mixture and the resulting mixture was stirred at 0° C. for 15 min. 2-bromoethan-1-amine hydrobromide (2.47 g, 12.2 mmol, 6.0 eq.) and TEA (2.46 g, 24.4 mmol, 12.0 eq.) was added into the above mixture. The resulting mixture was warmed up to room temperature and stirred for 1 hr. After completion, the reaction mixture was quenched with NH₄Cl (20 mL, sat. aq.) and extracted with EtOAc (30 mL×3). The combined organic layers were washed with brine (20 mL), dried over anhydrous Na₂SO₄, and concentrated under reduced pressure. The residue was purified by flash column chromatography on silica gel to afford (1R)-4-fluoro-5-nitro-2,3-dihydro-1H-indenyl P,P-bis[(2-bromoethyl)amino]phosphinate (350 mg, 0.72 mmol, 35%) as a yellow oil. LCMS (ESI): m/z 488 [M+H]⁺.

### STEP D: (R)-4-fluoro-5-nitro-2,3-dihydro-1H-inden-1-yl di(aziridin-1-yl)phosphinate

To a solution of (1R)-4-fluoro-5-nitro-2,3-dihydro-1H-indenyl P,P-bis[(2-bromoethyl)amino]phosphinate (5.3 g, 10.8 mmol, 1.0 eq.) and DIEA (14.0 g, 108.4 mmol, 10.0 eq.) in THF (100 mL) was added Ag₂O (25.1 g, 108.4 mmol, 10.0 eq.). The resulting mixture was stirred at 70° C. overnight. After completion, the reaction mixture was cooled to room temperature and filtered through a short pad of Celite®. The filtrate was concentrated under reduced pressure, the residue was purified by flash column chromatography on silica gel to give (R)-4-fluoro-5-nitro-2,3-dihydro-1H-inden-1-yl di(aziridin-1-yl)phosphinate (2.0 g, 6.11 mmol, 56%) as a yellow solid. LCMS (ESI): m/z 328 [M+H]⁺. Retention time: 5.343 min, 93% ee. Analytical method: Column: ChiralPak IB, 100×4.6 mm I.D., 5 μm; Mobile phase: A for CO₂ and B for Isopropyl alcohol (0.05% DEA); Gradient: 8 min @ 10% B; Flow rate: 2.5 mL/min; Column temperature: 40° C.

¹H NMR (400 MHz, CDCl₃) δ 8.12-7.79 (m, 1H), 7.45 (d, J=8.3 Hz, 1H), 6.17-5.86 (m, 1H), 3.33-3.11 (m, 1H), 3.03-2.86 (m, 1H), 2.79-2.58 (m, 1H), 2.47-1.98 (m, 9H). ¹⁹F NMR (376 MHz, CDCl₃) δ −121.63 (s). ³¹P NMR (162 MHz, CDCl₃) δ 29.91 (s).

### Illustration 39. Synthesis of (S)-4-fluoro-5-nitro-2,3-dihydro-1H-inden-1-yl di(aziridin-1-yl)phosphinate (Int.8)

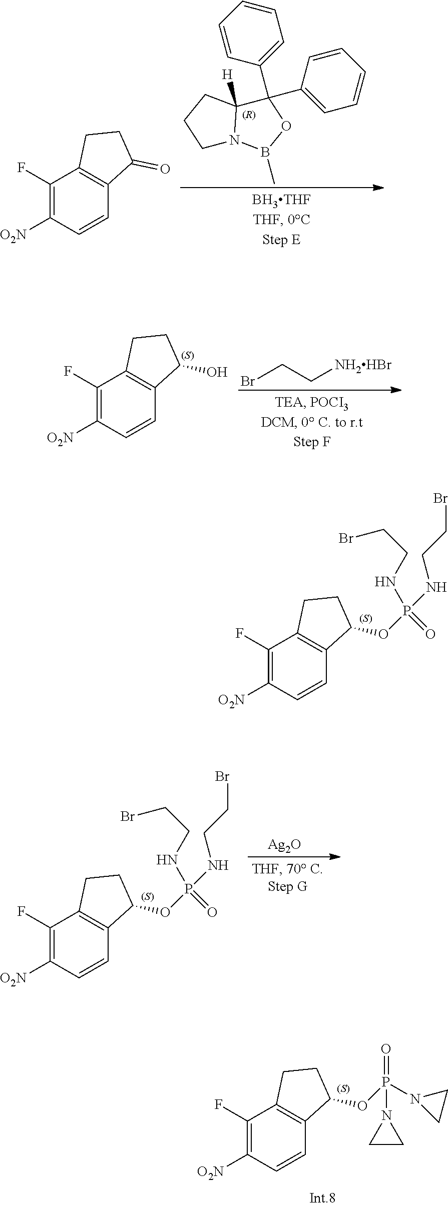

### STEP E: (S)-4-fluoro-5-nitro-2,3-dihydro-1H-inden-1-ol

To a solution of (R)-1-methyl-3,3-diphenyltetrahydro-1H,3H-pyrrolo[1,2-c][1,3,2]oxazaborole (5.96 g, 21.6 mmol, 0.6 eq.) in THF (160 mL) was added BH₃·THF (54 mL, 1 N, 53.8 mmol, 1.5 eq.) at 0° C. under nitrogen. The solution was stirred at 0° C. for 30 min. A solution of 4-fluoro-5-nitro-2,3-dihydro-1H-inden-1-one (7.0 g, 35.8 mmol, 1.0 eq.) in THF (160 mL) was added slowly to the above mixture at 0° C. After addition, the reaction mixture was stirred at 0° C. for 2 hrs. MeOH (160 mL) was added into the reaction mixture at 0° C., and the solution was stirred for 30 min. The mixture was concentrated under reduced pressure. The residue was purified by flash column chromatography on silica gel to give (S)-4-fluoro-5-nitro-2,3-dihydro-1H-inden-1-ol (6.3 g, 32.0 mmol, 89%) as a yellow solid. LCMS (EST): m/z 198 [M+H]⁺. Retention time: 1.838 min, 98% ee. Analytical method: Column: ChiralPak C-IG, 100×4.6 mm I.D., 5 μm; Mobile phase: A for CO₂ and B for methanol (0.05% DEA); Gradient: 8 min @ 20% B; Flow rate: 2.5 mL/min; Column temperature: 40 C.

### STEP F: (1S)-4-fluoro-5-nitro-2,3-dihydro-1H-indenylP,P-bis[(2-bromoethyl)amino]phosphinate

To a solution of POCl₃ (463 mg, 3.05 mmol, 1.5 eq.) in DCM (20 mL) was added TEA (615 mg, 6.09 mmol, 3.0 eq.) at 0° C. The resulting mixture was stirred at 0° C. for 15 min. (S)-4-fluoro-5-nitro-2,3-dihydro-1H-inden-1-ol (400 mg, 2.03 mmol, 1.0 eq.) was added into the above mixture and the resulting mixture was stirred at 0° C. for 15 min. 2-bromoethan-1-amine hydrobromide (2.47 g, 12.2 mmol, 6.0 eq.) and TEA (2.46 g, 24.4 mmol, 12.0 eq.) was added into the above mixture. The resulting mixture was warmed up to room temperature and stirred for 1 hr. After completion, the reaction mixture was quenched with NH₄Cl (20 mL, sat. aq.) and extracted with EtOAc (30 mL×3). The combined organic layers were washed with brine (20 mL), dried over anhydrous Na₂SO₄, and concentrated under reduced pressure. The residue was purified by flash column chromatography on silica gel to afford (1S)-4-fluoro-5-nitro-2,3-dihydro-1H-indenyl P,P-bis[(2-bromoethyl)amino]phosphinate (406 mg, 0.83 mmol, 41%) as a yellow oil. LCMS (ESI): m/z 488 [M+H]⁺.

### STEP G: (S)-4-fluoro-5-nitro-2,3-dihydro-1H-inden-1-yl di(aziridin-1-yl)phosphinate

To a solution of (1S)-4-fluoro-5-nitro-2,3-dihydro-1H-indenyl P,P-bis[(2-bromoethyl)amino]phosphinate (6.9 g, 14.1 mmol, 1.0 eq.) and DIEA (18.2 g, 141.1 mmol, 10.0 eq.) in THF (100 mL) was added Ag₂O (32.7 g, 141.1 mmol, 10.0 eq.). The resulting mixture was stirred at 70° C. overnight. After completion, the reaction mixture was cooled to room temperature and filtered through a short pad of Celite®. The filtrate was concentrated under reduced pressure, the residue was purified by flash column chromatography on silica gel to give (S)-4-fluoro-5-nitro-2,3-dihydro-1H-inden-1-yl di(aziridin-1-yl)phosphinate (3.0 g, 9.17 mmol, 65%) as a yellow solid. LCMS (ESI): m/z 328 [M+H]⁺. Retention time: 5.685 min, >99% ee. Analytical method: Column: ChiralPak IB, 100×4.6 mm I.D., 5 μm; Mobile phase: A for CO₂ and B for Isopropyl alcohol (0.05% DEA); Gradient: 8 min @ 10% B; Flow rate: 2.5 mL/min; Column temperature: 40° C. ¹H NMR (400 MHz, CDCl₃) δ 8.03-7.87 (m, 1H), 7.45 (d, J=8.3 Hz, 1H), 6.05-5.92 (m, 1H), 3.28-3.13 (m, 1H), 3.06-2.88 (m, 1H), 2.73-2.59 (m, 1H), 2.43-2.09 (m, 9H). ¹⁹F NMR (376 MHz, CDCl₃) δ −121.63 (s). ³¹P NMR (162 MHz, CDCl₃) δ 29.91 (s).

### Illustration 40. Synthesis of (S)-4-(3-fluoro-4-(4-methylpyrimidin-2-yl)phenoxy)-5-nitro-2,3-dihydro-1H-inden-1-yl di(aziridin-1-yl)phosphinate (267)

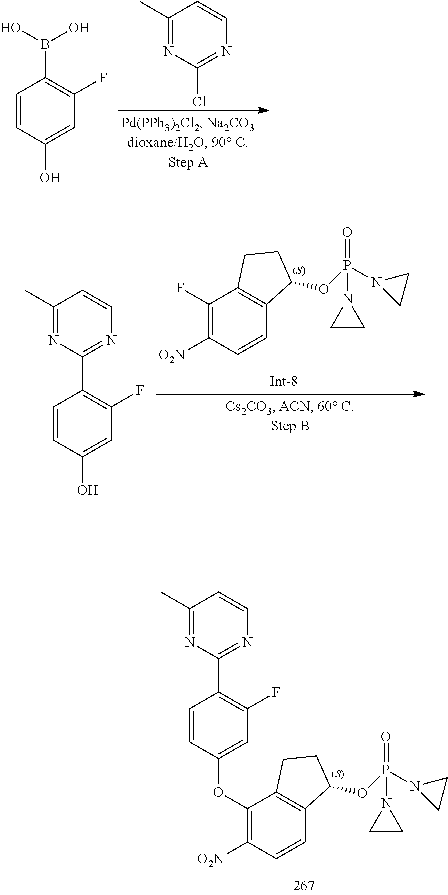

### STEP A: 3-fluoro-4-(4-methylpyrimidin-2-yl)phenol

To a solution of 2-chloro-4-methylpyrimidine (300 mg, 2.33 mmol, 1.0 eq.), (2-fluoro-4-hydroxyphenyl) boronic acid (364 mg, 2.33 mmol, 1.0 eq.) and Na₂CO₃ (742 mg, 7.00 mmol, 3.0 eq.) in a mixture of dioxane (10 mL) and H₂O (2 mL) was added Pd(PPh₃)₂Cl₂ (82 mg, 0.12 mmol, 0.05 eq.). The resulting mixture was stirred at 90° C. for 2 hrs. After completion, the reaction mixture was concentrated under reduced pressure. The residue was purified by flash column chromatography on silica gel to afford 3-fluoro-4-(4-methylpyrimidin-2-yl)phenol (130 mg, 0.64 mmol, 27%) as a white solid. LCMS (ESI): m/z 205 [M+H]⁺.

### STEP B: (S)-4-(3-fluoro-4-(4-methylpyrimidin-2-yl)phenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (25 mg, 76 μmol, 1.0 eq.) and 3-fluoro-4-(4-methylpyrimidin-2-yl)phenol (19 mg, 92 μmol, 1.2 eq.) in ACN (2 mL) was added Cs₂CO₃ (50 mg, 0.15 mmol, 2.0 eq.). The resulting mixture was stirred at 60° C. for 30 min. After completion, the reaction mixture was concentrated under reduced pressure. The residue was purified by prep-HPLC to give (S)-4-(3-fluoro-4-(4-methylpyrimidin-2-yl)phenoxy)-5-nitro-2,3-dihydro-1H-inden-1-yl di(aziridin-1-yl)phosphinate (267, 16.3 mg, 32 μmol, 42%) as a white solid.

Retention time: 2.811 min, 98% ee. LC-MS (ESI): m/z 512.2 [M+H]⁺; Analytical method: Column: ChiralPak AS, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C. ¹H NMR (400 MHz, CDCl₃) δ 8.69 (d, J=5.1 Hz, 1H), 8.03 (t, J=8.6 Hz, 1H), 7.94 (d, J=8.2 Hz, 1H), 7.57 (d, J=8.2 Hz, 1H), 7.09 (d, J=5.1 Hz, 1H), 6.78 (dd, J=8.7, 2.4 Hz, 1H), 6.70 (dd, J=11.7, 2.4 Hz, 1H), 6.04-6.00 (m, 1H), 2.95-2.88 (m, 1H), 2.69-2.55 (m, 5H), 2.30-2.17 (m, 9H). ³¹P NMR (162 MHz, CDCl₃) δ 29.90 (s).

### Illustration 41. Synthesis of (S)-4-(3-fluoro-4-(5-fluoropyrimidin-2-yl)phenoxy)-5-nitro-2,3-dihydro-1H-inden-1-yl di(aziridin-1-yl)phosphinate (268 and 269)

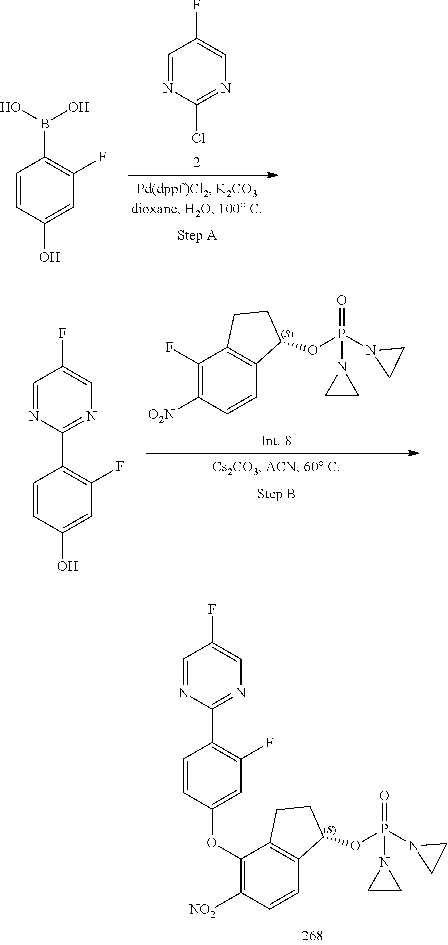

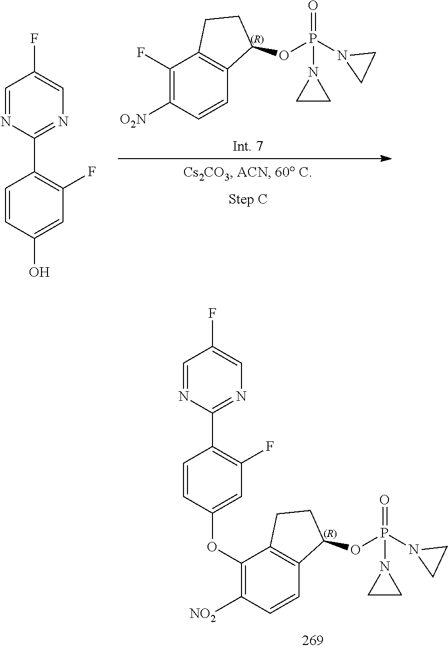

### STEP A: 3-fluoro-4-(5-fluoropyrimidin-2-yl)phenol

To a solution of (2-fluoro-4-hydroxyphenyl) boronic acid (400 mg, 2.57 mmol, 1.0 eq.), 2-chloro-5-fluoropyrimidine (374 mg, 2.82 mmol, 1.1 eq.) and K₂CO₃ (1.06 g, 7.70 mmol, 3.0 eq.) in dioxane (15 mL) and H₂O (3 mL) was added Pd(dppf)Cl₂ (94 mg, 0.13 mmol, 0.05 eq.). The resulting mixture was stirred at 100° C. for 2 hrs. After completion, the reaction mixture was concentrated under reduced pressure. The residue was purified by flash column chromatography on silica gel to afford 3-fluoro-4-(5-fluoropyrimidin-2-yl)phenol (100 mg, 0.48 mmol, 19%) as a yellow solid. LCMS (ESI): m/z 209 [M+H]⁺.

### STEP B: (S)-4-(3-fluoro-4-(5-fluoropyrimidin-2-yl)phenoxy)-5-nitro-2,3-dihydro-1H-inden-1-yl di(aziridin-1-yl)phosphinate (268)

To a solution of (S)-4-fluoro-5-nitro-2,3-dihydro-1H-inden-1-yl di(aziridin-1-yl)phosphinate, Int. 8 (20 mg, 61 μmol, 1.0 eq.) and 3-fluoro-4-(5-fluoropyrimidin-2-yl)phenol (15 mg, 73 mmol, 1.2 eq.) in ACN (1 mL) was added Cs₂CO₃ (40 mg, 0.12 mmol, 2.0 eq.). The resulting mixture was stirred at 60° C. overnight. After completion, the reaction mixture was concentrated under reduced pressure. The residue was purified by prep-HPLC to give (S)-4-(3-fluoro-4-(5-fluoropyrimidin-2-yl)phenoxy)-5-nitro-2,3-dihydro-1H-inden-1-yl di(aziridin-1-yl)phosphinate (268, 12.2 mg, 24 μmol, 31%) as a white solid. LC-MS (ESI): m/z 516.1 [M+H]⁺; Retention time: 1.389 min, >99% ee. Analytical method: Column: ChiralPak IH, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C. ¹H NMR (400 MHz, CDCl₃) δ 8.70 (s, 2H), 8.08-8.02 (m, 1H), 7.95 (d, J=8.2 Hz, 1H), 7.59 (d, J=8.2 Hz, 1H), 6.81-6.75 (m, 1H), 6.74-6.68 (m, 1H), 6.09-5.98 (m, 1H), 2.99-2.88 (m, 1H), 2.73-2.54 (m, 2H), 2.32-2.16 (m, 9H). ¹⁹F NMR (377 MHz, CDCl₃) δ −110.74 (s), −139.65 (s). ³¹P NMR (162 MHz, CDCl₃) δ 29.97 (s).

### STEP C: (R)-4-(3-fluoro-4-(5-fluoropyrimidin-2-yl)phenoxy)-5-nitro-2,3-dihydro-1H-inden-1-yl di(aziridin-1-yl)phosphinate, (269)

To a solution of (R)-4-fluoro-5-nitro-2,3-dihydro-1H-inden-1-yl di(aziridin-1-yl)phosphinate, Int. 7 (20 mg, 61 μmol, 1.0 eq.) and 3-fluoro-4-(5-fluoropyrimidin-2-yl)phenol (15 mg, 73 mmol, 1.2 eq.) in ACN (3 mL) was added Cs₂CO₃ (40 mg, 0.12 mmol, 2.0 eq.). The resulting mixture was stirred at 60° C. overnight. After completion, the reaction mixture was concentrated under reduced pressure. The residue was purified by prep-HPLC to give (R)-4-(3-fluoro-4-(5-fluoropyrimidin-2-yl)phenoxy)-5-nitro-2,3-dihydro-1H-inden-1-yl di(aziridin-1-yl)phosphinate (11.3 mg, 22 μmol, 36%) as a white solid. LC-MS (ESI): m/z 516.1 [M+H]⁺; Retention time: 1.766 min, >99% ee. Analytical method: Column: ChiralPak IH, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C. ¹H NMR (400 MHz, CDCl₃) δ 8.70 (s, 2H), 8.08-8.02 (m, 1H), 7.95 (d, J=8.2 Hz, 1H), 7.58 (d, J=8.2 Hz, 1H), 6.80-6.76 (m, 1H), 6.74-6.69 (m, 1H), 6.08-5.98 (m, 1H), 2.98-2.89 (m, 1H), 2.72-2.55 (m, 2H), 2.33-2.16 (m, 9H). ¹⁹F NMR (377 MHz, CDCl₃) δ −110.74 (s), −139.65 (s). ³¹P NMR (162 MHz, CDCl₃) δ 29.97 (s).

TABLE 9
Characterization of compounds (see Illustration 36-41)

| Compound | | |
|---|---|---|
| No. | Structure | LCMS; ¹H NMR (ppm); Retention time |
| 270 | 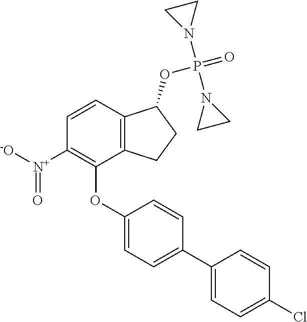 | LCMS(ESI): m/z 512 [M + H]⁺; ¹H NMR (400 MHz, CDCl₃) δ7.93 (d, J = 8.2 Hz, 1H), 7.55 (d, J = 8.1 Hz, 1H), 7.61-7.48 (m, 4H), 7.41 (d, J = 8.5 Hz, 2H), 6.95 (d, J = 8.7 Hz, 2H), 6.06-6.01 (m, 1H), 3.00-2.88 (m, 1H), 2.75-2.54 (m, 2H), 2.38-2.17 (m, 9H). ee: >99% Retention time: 8.240 min (Method 3). |
| 271 | 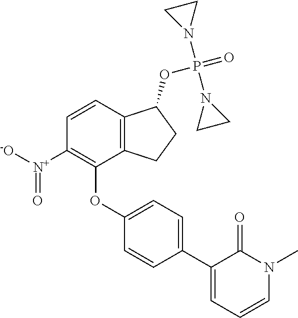 | LCMS(ESI): m/z 509.1 [M + H]⁺; ¹H NMR (400 MHz, CDCl₃) δ 7.89 (d, J = 8.2 Hz, 1H), 7.69-7.63 (m, 2H), 7.53-7.43 (m, 2H), 7.30 (dd, J = 6.7, 2.0 Hz, 1H), 6.91-6.86 (m, 2H), 6.25 (t, J = 6.8 Hz, 1H), 6.03-5.96 (m, 1H), 3.61 (s, 3H), 2.93-2.84 (m, 1H), 2.68-2.49 (m, 2H), 2.30-2.15 (m, 9H).. ee: >99% Retention time: 4.359 min (Method 4). |
| 272 | 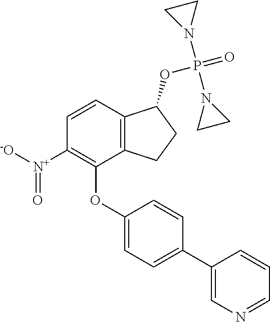 | LCMS(ESI): m/z 479.2 [M + H]⁺; ¹H NMR (400 MHz, CDCl₃) δ 8.81 (d, J = 1.9 Hz, 1H), 8.58 (dd, J = 4.8, 1.5 Hz, 1H), 7.92 (d, J = 8.2 Hz, 1H), 7.88-7.79 (m, 1H), 7.57-7.48 (m, 3H), 7.39-7.31 (m, 1H), 7.02-6.92 (m, 2H), 6.07-5.98 (m, 1H), 2.99-2.87 (m, 1H), 2.73-2.52 (m, 2H), 2.33-2.15 (m, 9H). ee: >99% Retention time: 4.860 min (Method 6). |
| 273 | 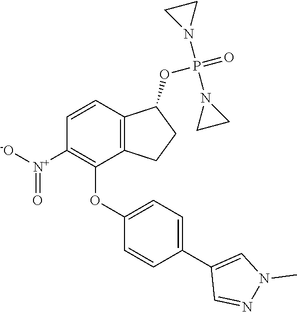 | LCMS(ESI): m/z 482.2 [M + H]⁺; ¹H NMR (400 MHz, CDCl₃) δ 7.88 (d, J = 8.2 Hz, 1H), 7.69 (s, 1H), 7.55 (s, 1H), 7.50 (d, J = 8.2 Hz, 1H), 7.41-7.36 (m, 2H), 6.90-6.83 (m, 2H), 6.05-5.96 (m, 1H), 3.93 (s, 3H), 2.92-2.82 (m, 1H), 2.65-2.51 (m, 2H), 2.32-2.17 (m, 9H). ee: >99% Retention time: 3.070 min (Method 7). |
| 274 | 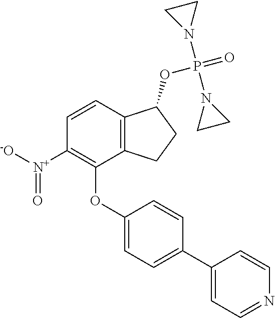 | LCMS(ESI): m/z 479.2 [M + H]⁺; ¹H NMR (400 MHz, CDCl₃) δ 8.65-8.59 (m, 2H), 7.93 (d, J = 8.2 Hz, 1H), 7.62-7.57 (m, 2H), 7.55 (d, J = 8.2 Hz, 1H), 7.48-7.44 (m, 2H), 7.01-6.94 (m, 2H), 6.06-5.99 (m, 1H), 2.97-2.87 (m, 1H), 2.71-2.56 (m, 2H), 2.31-2.14 (m, 9H). ee: 88% Retention time: 4.683 min (Method 4). |
| 275 | 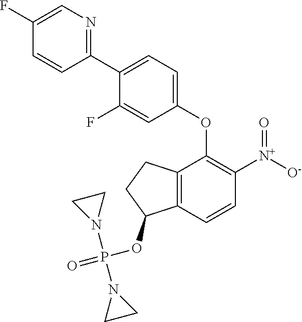 [SMILES: O=[N+]([O-])c1ccc2c(c1Oc1ccc(-c3ccc(F)cn3)c(F)c1)CC[C@@H]2OP(=O)(N1CC1)N1CC1] | LCMS(ESI): m/z 515 [M + H] ; ¹H NMR (400 MHz, CDCl₃) δ 8.54 (d, J = 2.9 Hz, 1H), 7.99-7.84 (m, 2H), 7.79-7.69 (m, 1H), 7.57 (d, J = 8.2 Hz, 1H), 7.51-7.39 (m, 1H), 6.83-6.64 (m, 2H), 6.08-5.95 (m, 1H), 2.99-2.84 (m, 1H), 2.74-2.51 (m, 2H), 2.34-2.12 (m, 9H). ee: 95% Retention time: 3.188 min (Method 13). |
| 276 | 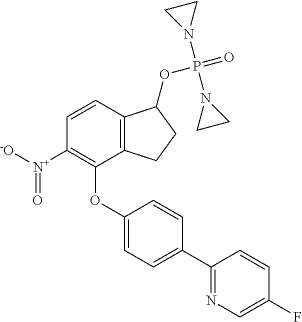 | LCMS(ESI): m/z 497.1 [M + H]⁺; ¹H NMR (400 MHz, CDCl₃) δ 8.51 (d, J = 2.9 Hz, 1H), 7.92-7.87 (m, 3H), 7.68-7.64 (m, 1H), 7.53 (d, J = 8.3 Hz, 1H), 7.48-7.43 (m, 1H), 6.98-6.94 (m, 2H), 6.06-5.96 (m, 1H), 2.94-2.84 (m, 1H), 2.66-2.52 (m, 2H), 2.30-2.14 (m, 9H). |
| 277 | 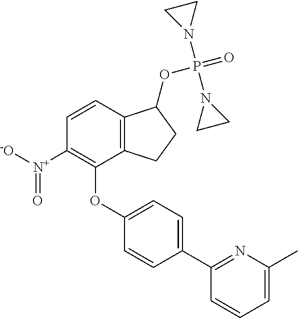 | LCMS(ESI): m/z 493.2 [M + H]⁺; ¹H NMR (400 MHz, CDCl₃) δ 7.96-7.87 (m, 3H), 7.63 (t, J = 7.7 Hz, 1H), 7.52 (d, J = 8.2 Hz, 1H), 7.46 (d, J = 7.8 Hz, 1H), 7.08 (d, J = 7.6 Hz, 1H), 6.96 (d, J = 8.8 Hz, 2H), 6.00 (dd, J = 12.1, 7.0 Hz, 1H), 2.91- 2.83 (m, 1H), 2.66-2.49 (m, 5H), 2.29-2.17 (m, 9H). |
| 278 | 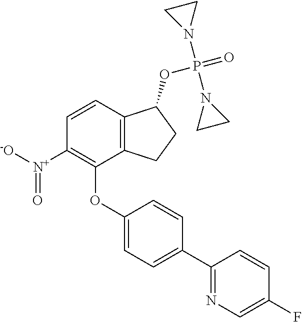 | LCMS(ESI): m/z 497.2 [M + H]⁺; ¹H NMR (400 MHz, CDCl₃) δ 8.51 (d, J = 2.8 Hz, 1H), 7.90 (t, J = 8.4 Hz, 3H), 7.69-7.64 (m, 1H), 7.53 (d, J = 8.2 Hz, 1H), 7.49-7.43 (m, 1H), 6.96 (d, J = 8.8 Hz, 2H), 6.08-5.91 (m, 1H), 2.93-2.83 (m, 1H), 2.67-2.51 (m, 2H), 2.29-2.14 (m, 9H). ee: 94% Retention time: 2.278 min (Method 7). |
| 279 | 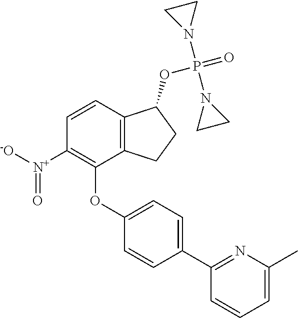 | LCMS(ESI): m/z 493.3 [M + H]⁺; ¹H NMR (400 MHz, CDCl₃) δ 7.96-7.87 (m, 3H), 7.62 (t, J = 7.7 Hz, 1H), 7.52 (d, J = 8.2 Hz, 1H), 7.46 (d, J = 7.8 Hz, 1H), 7.27 (s, 1H), 7.07 (d, J = 7.6 Hz, 1H), 6.95 (d, J = 8.7 Hz, 2H), 6.00 (dd, J = 12.1, 6.8 Hz, 1H), 2.91-2.82 (m, 1H), 2.66-2.49 (m, 5H), 2.29-2.17 (m, 9H). ee: 98% Retention time: 2.267 min (Method 7). |
| 280 | 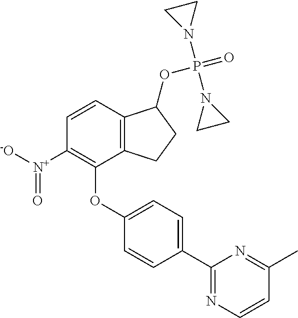 | LCMS(ESI): m/z 494.1 [M + H]⁺; ¹H NMR (400 MHz, CDCl₃) δ 8.61 (d, J = 5.1 Hz, 1H), 8.41 (d, J = 8.9 Hz, 2H), 7.91 (d, J = 8.2 Hz, 1H), 7.54 (s, 1H), 7.02 (d, J = 5.1 Hz, 1H), 6.99-6.94 (m, 2H), 6.05-5.96 (m, 1H), 2.92-2.82 (m, 1H), 2.66-2.58 (m, 1H), 2.57 (s, 3H), 2.56-2.48 (m, 1H), 2.29-2.16 (m, 9H). |
| 281 | 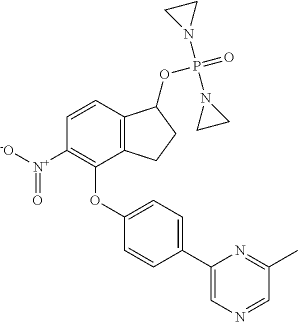 | LCMS(ESI): m/z 494.2 [M + H]⁺; ¹H NMR (400 MHz, CDCl₃) δ 8.77 (s, 1H), 8.37 (s, 1H), 8.00-7.94 (m, 2H), 7.92 (d, J = 8.2 Hz, 1H), 7.55 (d, J = 8.2 Hz, 1H), 7.01-6.97 (m, 2H), 6.05-5.98 (m, 1H), 2.95-2.83 (m, 1H), 2.68-2.51 (m, 5H), 2.29- 2.17 (m, 9H). |
| 282 | 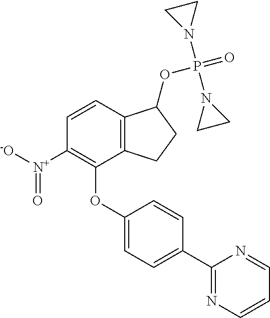 | LCMS(ESI): m/z 480.1 [M + H]⁺; ¹H NMR (400 MHz, CDCl₃) δ 8.77 (d, J = 4.8 Hz, 2H), 8.45-8.41 (m, 1H), 8.41-8.39 (m, 1H), 7.92 (d, J = 8.2 Hz, 1H), 7.54 (d, J = 8.2 Hz, 1H), 7.16 (t, J = 4.8 Hz, 1H), 7.00-6.98 (m, 1H), 6.97-6.95 (m, 1H), 6.06-5.95 (m, 1H), 2.94-2.83 (m, 1H), 2.68-2.50 (m, 2H), 2.32-2.12 (m, 9H). |
| 283 | 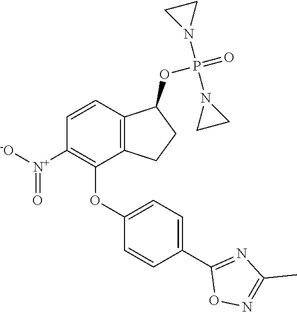 | LCMS(ESI): m/z 484.1 [M + H]⁺; ¹H NMR (400 MHz, CDCl₃) δ 8.08 (d, J = 8.8 Hz, 2H), 7.95 (d, J = 8.2 Hz, 1H), 7.59 (d, J = 8.2 Hz, 1H), 6.99 (d, J = 8.8 Hz, 2H), 6.03 (d, J = 5.4 Hz, 1H), 2.97-2.85 (m, 1H), 2.71-2.54 (m, 2H), 2.46 (s, 3H), 2.36- 2.14 (m, 9H). ee: >99% Retention time: 1.977 min (Method 37). |
| 284 | 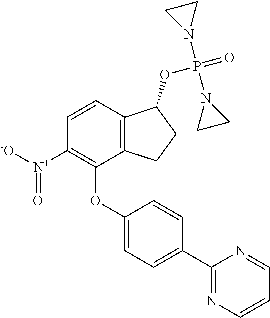 | LCMS(ESI): m/z 480.2 [M + H]⁺; ¹H NMR (400 MHz, CDCl₃) δ 8.77 (d, J = 4.8 Hz, 2H), 8.46-8.38 (m, 2H), 7.92 (d, J = 8.2 Hz, 1H), 7.54 (d, J = 8.2 Hz, 1H), 7.19-7.13 (m, 1H), 7.02-6.94 (m, 2H), 6.06-5.96 (m, 1H), 2.96-2.81 (m, 1H), 2.70- 2.48 (m, 2H), 2.30-2.16 (m, 9H). ee: >99% Retention time: 4.197 min (Method 29). |
| 285 | 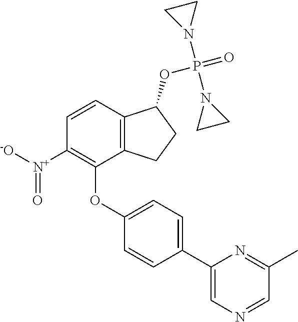 | LCMS(ESI): m/z 494.2 [M + H]⁺; ¹H NMR (400 MHz, CDCl₃) δ 8.77 (s, 1H), 8.37 (s, 1H), 8.00-7.89 (m, 3H), 7.55 (d, J = 8.2 Hz, 1H), 6.99 (d, J = 8.8 Hz, 2H), 6.02 (dd, J = 12.2, 6.9 Hz, 1H), 2.94-2.84 (m, 1H), 2.68-2.51 (m, 5H), 2.29-2.17 (m, 9H). ee: >99% Retention time: 6.505 min (Method 39). |
| 286 | 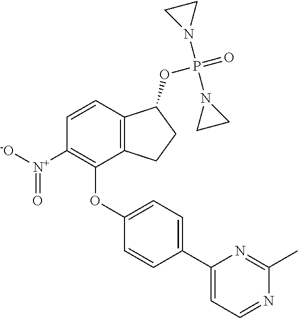 | LCMS(ESI): m/z 494.1 [M + H]⁺; ¹H NMR (400 MHz, CDCl₃) δ 8.65 (d, J = 5.4 Hz, 1H), 8.05 (d, J = 8.8 Hz, 2H), 7.93 (d, J = 8.2 Hz, 1H), 7.56 (d, J = 8.3 Hz, 1H), 7.45 (d, J = 5.3 Hz, 1H), 6.98 (d, J = 8.8 Hz, 2H), 6.10-5.97 (m, 1H), 2.94-2.85 (m, 1H), 2.78 (s, 3H), 2.69-2.51 (m, 2H), 2.31-2.16 (m, 9H). ee: 96% Retention time: 5.912 min (Method 39). |
| 287 | 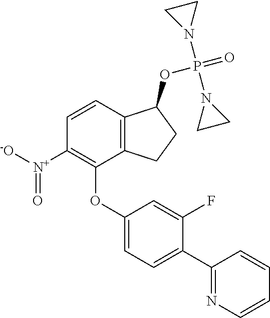 | LCMS(ESI): m/z 497.1 [M + H]⁺; ¹H NMR (400 MHz, CDCl₃) δ 8.72-8.65 (m, 1H), 8.03-7.89 (m, 2H), 7.76 (d, J = 3.6 Hz, 2H), 7.57 (d, J = 8.3 Hz, 1H), 7.24 (s, 1H), 6.82- 6.66 (m, 2H), 6.07-5.83 (m, 1H), 3.00-2.85 (m, 1H), 2.73-2.46 (m, 2H), 2.31-2.17 (m, 9H). ee: >99% Retention time: 1.384 min (Method 2). |
| 288 | 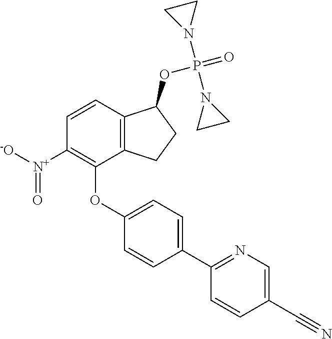 | LCMS(ESI): m/z 504.1 [M + H]⁺; ¹H NMR (400 MHz, CDCl₃) δ 9.07 (d, J = 1.9 Hz, 1H), 8.46-8.33 (m, 1H), 8.23-8.13 (m, 3H), 8.08 (d, J = 8.2 Hz, 1H), 7.59 (d, J = 8.2 Hz, 1H), 7.07 (d, J = 8.8 Hz, 2H), 6.08-5.87 (m, 1H), 2.81-2.71 (m, 1H), 2.65-2.54 (m, 2H), 2.18-2.09 (m, 9H). ee: 98% Retention time: 3.830 min (Method 7). |
| 289 | 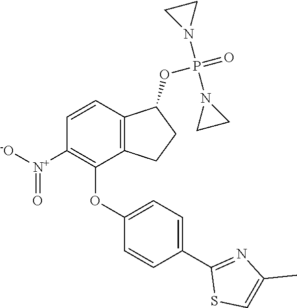 | LCMS(ESI): m/z 499.0 [M + H]⁺; ¹H NMR (400 MHz, CDCl₃) δ7.94-7.85 (m, 3H), 7.54 (d, J = 8.2 Hz, 1H), 6.92 (d, J = 8.8 Hz, 2H), 6.84 (s, 1H), 6.04-5.97 (m, 1H), 2.92- 2.82 (m, 1H), 2.69-2.51 (m, 2H), 2.49 (s, 3H), 2.28-2.17 (m, 9H). ee: >99% Retention time: 2.347 min (Method 44). |
| 290 | 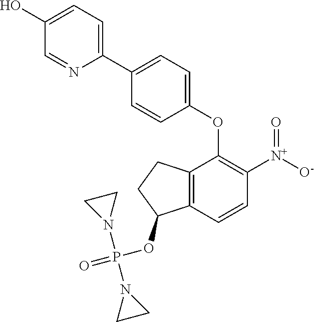 [SMILES: O=[N+]([O-])c1ccc2c(c1Oc1ccc(-c3ccc(O)cn3)cc1)CC[C@@H]2OP(=O)(N1CC1)N1CC1] | LCMS(ESI): m/z 495.2 [M + H]⁺; ¹H NMR (400 MHz, CDCl₃) δ 8.30 (d, J = 2.9 Hz, 1H), 7.93 (d, J = 8.2 Hz, 1H), 7.82 (d, J = 8.7 Hz, 2H), 7.62-7.55 (m, 2H), 7.23 (dd, J = 8.8, 2.9 Hz, 1H), 6.91 (d, J = 8.7 Hz, 2H), 6.10 (s, 1H), 6.04-5.99 (m, 1H), 2.94-2.87 (m, 1H), 2.72-2.50 (m, 2H), 2.34-2.15 (m, 9H). ee: 93% Retention time: 3.516 min (Method 7). |
| 291 | 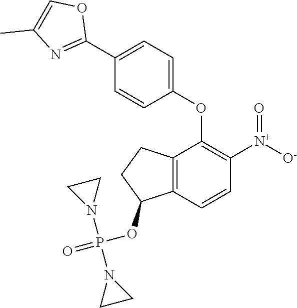 [SMILES: Cc1coc(-c2ccc(Oc3c([N+](=O)[O-])ccc4c3CC[C@@H]4OP(=O)(N3CC3)N3CC3)cc2)n1] | LCMS(ESI): m/z 483.2 [M + H]⁺; ¹H NMR (400 MHz, CDCl₃) δ 8.01-7.94 (m, 2H), 7.92 (d, J = 8.2 Hz, 1H), 7.54 (d, J = 8.2 Hz, 1H), 7.40 (d, J = 1.2 Hz, 1H), 6.98-6.89 (m, 2H), 6.06-5.97 (m, 1H), 2.93-2.81 (m, 1H), 2.69-2.48 (m, 2H), 2.33- 2.12 (m, 12H). ee: 96% Retention time: 3.699 min (Method 13). |

### Illustration 42. Synthesis of (S)-4-(3-(3-methyl-1,2,4-thiadiazol-5-yl)phenoxy)-5-nitro-2,3-dihydro-1H-inden-1-yl di(aziridin-1-yl)phosphinate (292)

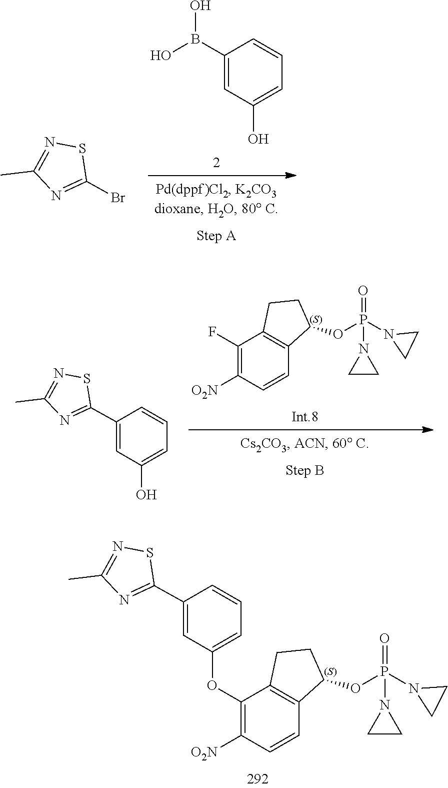

### STEP A: 3-(3-methyl-1,2,4-thiadiazol-5-yl)phenol

To a solution of 5-bromo-3-methyl-1,2,4-thiadiazole (500 mg, 2.79 mmol, 1.0 eq.) and (3-hydroxyphenyl) boronic acid (385 mg, 2.79 mmol), 1.0 eq.) and K₂CO₃ (772 mg, 5.59 mmol, 2.0 eq.) in dioxane/H₂O (30 mL/6 mL) was added Pd(dppf)Cl₂ (204 mg, 0.28 mmol, 0.1 eq.). The resulting mixture was stirred at 80° C. for 2 hrs. After completion, the reaction mixture was concentrated under reduced pressure. The residue was purified by flash column chromatography on silica gel to afford 3-(3-methyl-1,2,4-thiadiazol-5-yl)phenol (230 mg, 1.20 mmol, 43%) as a yellow solid. LCMS (ESI): m/z 193.0 [M+H]⁺.

### STEP B: (S)-4-(3-(3-methyl-1,2,4-thiadiazol-5-yl)phenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (20 mg, 61 μmol, 1.0 eq.) and 3-(3-methyl-1,2,4-thiadiazol-5-yl)phenol (13 mg, 67 μmol, 1.1 eq.) in ACN (3 mL) was added Cs₂CO₃ (60 mg, 0.18 mmol, 2.0 eq.). The resulting mixture was stirred at 60° C. for 2 hrs. After completion, the reaction mixture was concentrated under reduced pressure. The residue was purified by prep-HPLC to give (S)-4-(3-(3-methyl-1,2,4-thiadiazol-5-yl)phenoxy)-5-nitro-2,3-dihydro-1H-inden-1-yl di(aziridin-1-yl)phosphinate, 292 (15.4 mg, 31 μmol, 50%) as a pink solid. LCMS (ESI): m/z 500.1 [M+H]⁺. Retention time: 3.967 min, 95% ee. Analytical method: Column: ChiralPak IG, 100;Á4.6 mm I.D., 5 μm, Mobile phase: A for CO₂ and B for methanol (0.05% DEA), Gradient: 8 min @B 40%, Flow rate: 2.5 mL/min, Back pressure: 100 bar, Column temperature: 35° C. ¹H NMR (400 MHz, CDCl₃) δ 7.94 (d, J=8.2 Hz, 1H), 7.62 (d, J=7.7 Hz, 1H), 7.57 (d, J=8.2 Hz, 1H), 7.52 (s, 1H), 7.45-7.39 (m, 1H), 7.00-6.95 (m, 1H), 6.06-5.98 (m, 1H), 2.95-2.85 (m, 1H), 2.71 (s, 3H), 2.66-2.54 (m, 2H), 2.28-2.18 (m, 9H). ³¹P NMR (162 MHz, CDCl₃) δ 30.01 (s).

### Illustration 43. Synthesis of (S)-4-(3-(5-fluoropyrimidin-2-yl)-4-methoxyphenoxy)-5-nitro-2,3-dihydro-1H-inden-1-yl di(aziridin-1-yl)phosphinate (293)

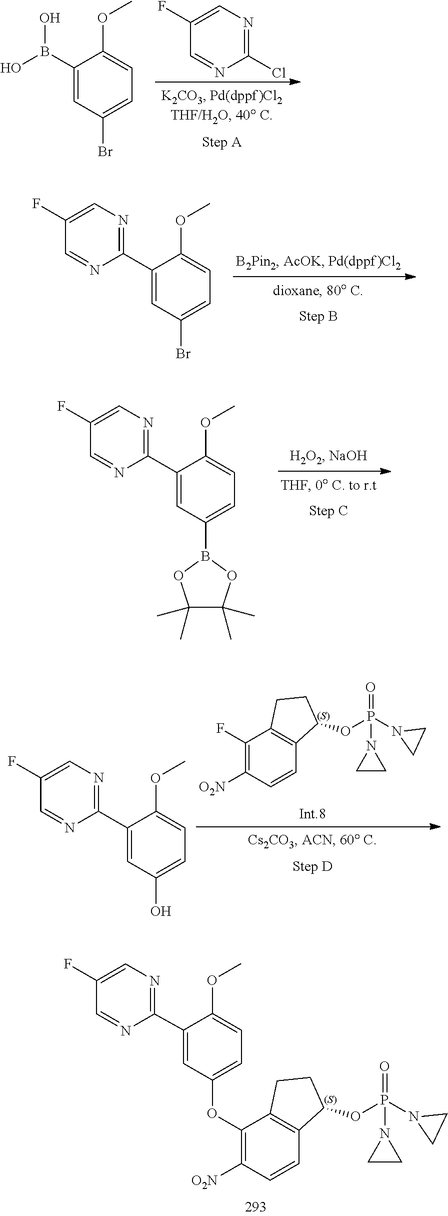

### STEP A: 2-(5-bromo-2-methoxyphenyl)-5-fluoropyrimidine

To a solution of 2-chloro-5-fluoropyrimidine (1 g, 7.58 mmol, 1.0 eq.) and (5-bromo-2-methoxyphenyl) boronic acid (2.62 g, 11.4 mmol, 1.5 eq.) and K₂CO₃ (3.14 g, 22.7 mmol, 3.0 eq.) in THF/H₂O (20 mL/4 mL) was added Pd(dppf)Cl₂ (554 mg, 0.76 mmol, 0.1 eq.). The resulting mixture was stirred at 40° C. for 18 hrs. After completion, the reaction mixture was concentrated under reduced pressure. The residue was purified by flash column chromatography on silica gel to afford 2-(5-bromo-2-methoxyphenyl)-5-fluoropyrimidine (500 mg, 1.77 mmol, 23%) as a yellow solid. LCMS (ESI): m/z 283 [M+H]⁺.

### STEP B: 5-fluoro-2-(2-methoxy-5-(4,4,5,5-tetramethyl-1,3,2-dioxaborolan-2-yl)phenyl)pyrimidine

To a solution of 2-(5-bromo-2-methoxyphenyl)-5-fluoropyrimidine (500 mg, 1.77 mmol, 1.0 eq.) and 4,4,4′,4′,5,5,5′,5′-octamethyl-2,2′-bi(1,3,2-dioxaborolane) (674 mg, 2.66 mmol, 1.5 eq.) and KOAc (520 mg, 5.31 mmol, 3.0 eq.) in dioxane (20 mL) was added Pd(dppf)Cl₂ (132 mg, 0.18 mmol, 0.1 eq.). The resulting mixture was stirred at 80° C. for 2 hrs. After completion, the reaction mixture was concentrated under reduced pressure. The residue was purified by flash column chromatography on silica gel to afford 5-fluoro-2-(2-methoxy-5-(4,4,5,5-tetramethyl-1,3,2-dioxaborolan-2-yl)phenyl)pyrimidine (550 mg, 1.67 mmol, 94%) as a pink solid. LCMS (ESI): m/z 331.0 [M+H]⁺.

### STEP C: 3-(5-fluoropyrimidin-2-yl)-4-methoxyphenol

To a solution of 5-fluoro-2-(2-methoxy-5-(4,4,5,5-tetramethyl-1,3,2-dioxaborolan-2-yl)phenyl)pyrimidine (700 mg, 2.11 mmol, 1.0 eq.) in THF (10 mL) was added KOH (236 mg, 4.21 mmol, 2.0 eq.) at 0° C. H₂O₂ (477 mg×30 wt %, 4.21 mmol, 2.0 eq.) was added to the mixture dropwise at 0° C. The mixture was warmed up to 25° C. and stirred for 1 hr. After completion, the reaction mixture was quenched by adding Na₂S₂O₃ (15 wt %, aq., 10 mL) and stirred for 1 hr, then separated. The aqueous layer was neutralized carefully with HCl (aq., 1M) until the pH was adjusted to pH=3-4. The resulting mixture was extracted with DCM (20 mL). The organic layers were combined and washed with brine (20 mL), dried over anhydrous Na₂SO₄, then concentrated under reduced pressure. The residue was purified by flash column chromatography on silica gel to afford 3-(5-fluoropyrimidin-2-yl)-4-methoxyphenol (400 mg, 1.82 mmol, 86%) as a yellow solid. LCMS (ESI): m/z 221 [M+H]⁺.

### STEP D: (S)-4-(3-(5-fluoropyrimidin-2-yl)-4-methoxyphenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (20 mg, 61 μmol, 1.0 eq.) and 3-(5-fluoropyrimidin-2-yl)-4-methoxyphenol (15 mg, 67 μmol, 1.1 eq.) in ACN (3 mL) was added Cs₂CO₃ (60 mg, 0.18 mmol, 2.0 eq.). The resulting mixture was stirred at 60° C. for 2 hrs. After completion, the reaction mixture was concentrated under reduced pressure. The residue was purified by prep-HPLC to give (S)-4-(3-(5-fluoropyrimidin-2-yl)-4-methoxyphenoxy)-5-nitro-2,3-dihydro-1H-inden-1-yl di(aziridin-1-yl)phosphinate 293 (12.8 mg, 26 μmol, 42%) as a white solid. LCMS (ESI): m/z 528.2 [M+H]⁺. Retention time: 1.924 min, 97% ee. Analytical method: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. ¹H NMR (400 MHz, CDCl₃) δ 8.69 (s, 2H), 7.86 (d, J=8.2 Hz, 1H), 7.47 (d, J=8.2 Hz, 1H), 7.30 (d, J=2.4 Hz, 1H), 6.99-6.92 (m, 2H), 6.01-5.94 (m, 1H), 3.85 (s, 3H), 2.91-2.80 (m, 1H), 2.64-2.49 (m, 2H), 2.27-2.17 (m, 9H). ¹⁹F NMR (376 MHz, CDCl₃) δ −139.87 (s). ³¹P NMR (162 MHz, CDCl₃) δ 30.00 (s).

### Illustration 44. Synthesis of (S)-4-(4-methoxy-3-(pyrimidin-2-yl)phenoxy)-5-nitro-2,3-dihydro-1H-inden-1-yl di(aziridin-1-yl)phosphinate (294)

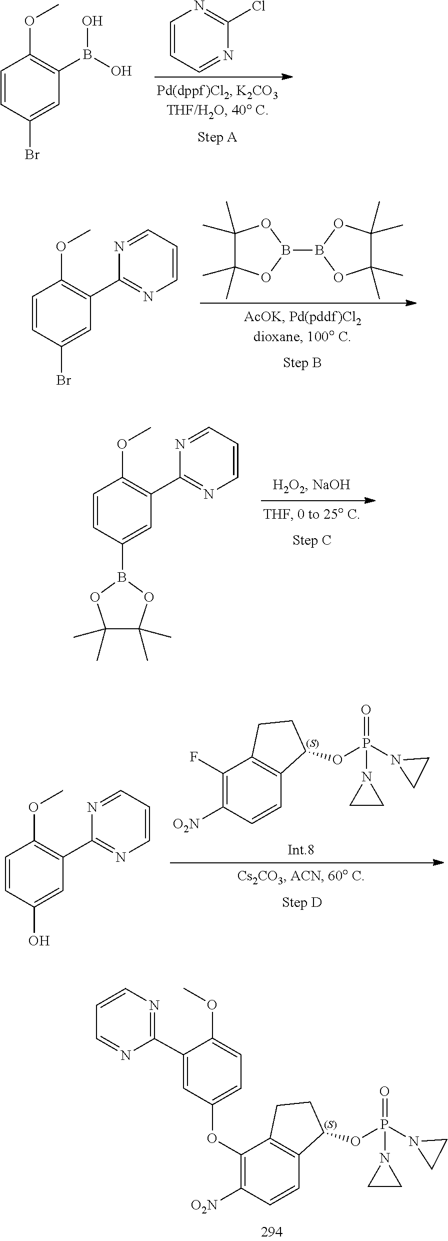

### STEP A: 2-(5-bromo-2-methoxyphenyl)pyrimidine

To a solution of (5-bromo-2-methoxyphenyl) boronic acid (500 mg, 2.17 mmol, 1.0 eq.), 2-chloropyrimidine (273 mg, 2.38 mmol, 1.1 eq.) and K₂CO₃ (898 mg, 6.50 mmol, 3.0 eq.) in THF (20 mL) and H₂O (4 mL) was added Pd(dppf)Cl₂ (79 mg, 0.11 mmol, 0.05 eq.). The resulting mixture was stirred at 40° C. under N₂ for 1 hr. After completion, the reaction mixture was concentrated under reduced pressure. The residue was purified by flash column chromatography on silica gel to afford 2-(5-bromo-2-methoxyphenyl)pyrimidine (420 mg, 1.58 mmol, 73%) as a yellow solid. LCMS (ESI): m/z 265 [M+H]⁺.

### STEP B: 2-(2-methoxy-5-(4,4,5,5-tetramethyl-1,3,2-dioxaborolan-2-yl)phenyl)pyrimidine

To a solution of 2-(5-bromo-2-methoxyphenyl)pyrimidine (300 mg, 1.13 mmol, 1.0 eq.) and 4,4,4′,4′,5,5,5′,5′-octamethyl-2,2′-bi (1,3,2-dioxaborolane) (344 mg, 1.36 mmol, 1.2 eq.) in dioxane (15 mL) were added KOAc (333 mg, 3.40 mmol, 3.0 eq.) and Pd(dppf)Cl₂ (83 mg, 0.11 mmol, 0.1 eq.) at 25° C. The mixture was headed to 100° C. and stirred for 12 hrs under N₂ atmosphere. After completion, the reaction mixture was concentrated under reduced pressure. The residue was purified by flash column chromatography on silica gel to afford 2-(2-methoxy-5-(4,4,5,5-tetramethyl-1,3,2-dioxaborolan-2-yl)phenyl)pyrimidine (140 mg, 0.45 mmol, 40%) as a yellow solid. LCMS (ESI): m/z 313 [M+H]⁺.

### STEP C: 4-methoxy-3-(pyrimidin-2-yl)phenol

To a solution of 2-(2-methoxy-5-(4,4,5,5-tetramethyl-1,3,2-dioxaborolan-2-yl)phenyl)pyrimidine (300 mg, 0.96 mmol, 1.0 eq.) in THF (4 mL) were added KOH (108 mg, 1.92 mmol, 2.0 eq.) and H₂O₂ (218 mg, 1.92 mmol, 2.0 eq.) at 0° C. dropwise. After addition, the mixture was warmed up to 25° C. and stirred for 3 hrs. After completion, the reaction mixture was cooled down in an ice bath, then neutralized carefully with HCl (aq., 2 N) until the pH was adjusted to pH=2. The resulting mixture was extracted with DCM (20 mL×3), and the combined organic layers were washed with brine (10 mL), dried over with anhydrous Na₂SO₄, then concentrated under reduced pressure. The residue was purified by flash column chromatography on silica gel to afford 4-methoxy-3-(pyrimidin-2-yl)phenol (100 mg, 0.50 mmol, 51%) as a yellow solid. LCMS (ESI): m/z 203 [M+H]⁺.

### STEP D: (S)-4-(4-methoxy-3-(pyrimidin-2-yl)phenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (25 mg, 77 μmol, 1.0 eq.) and 4-methoxy-3-(pyrimidin-2-yl)phenol (19 mg, 92 μmol, 1.2 eq.) in ACN (1 mL) was added Cs₂CO₃ (50 mg, 0.15 mmol, 2.0 eq.). The reaction mixture was stirred at 60° C. for 1 hr. After completion, the reaction mixture was purified by prep-HPLC to give (S)-4-(4-methoxy-3-(pyrimidin-2-yl)phenoxy)-5-nitro-2,3-dihydro-1H-inden-1-yl di(aziridin-1-yl)phosphinate 294 (21 mg, 41 μmol, 53%) as a yellow solid. LCMS (ESI): m/z 510.1 [M+H]⁺. ¹H NMR (400 MHz, CDCl₃) δ 8.86-8.81 (m, 2H), 7.85 (d, J=8.2 Hz, 1H), 7.46 (d, J=8.2 Hz, 1H), 7.36-7.32 (m, 1H), 7.23 (t, J=4.9 Hz, 1H), 6.99-6.92 (m, 2H), 6.02-5.93 (m, 1H), 3.85 (s, 3H), 2.90-2.81 (m, 1H), 2.67-2.48 (m, 2H), 2.28-2.14 (m, 9H). ³¹P NMR (162 MHz, CDCl₃) δ 29.91 (s). Retention time: 2.393 min, 96% ee. Analytical method: ChiralPak IB, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

### Illustration 45. Synthesis of (S)-4-(3-(5-methyloxazol-2-yl)phenoxy)-5-nitro-2,3-dihydro-1H-inden-1-yl di(aziridin-1-yl)phosphinate (295)

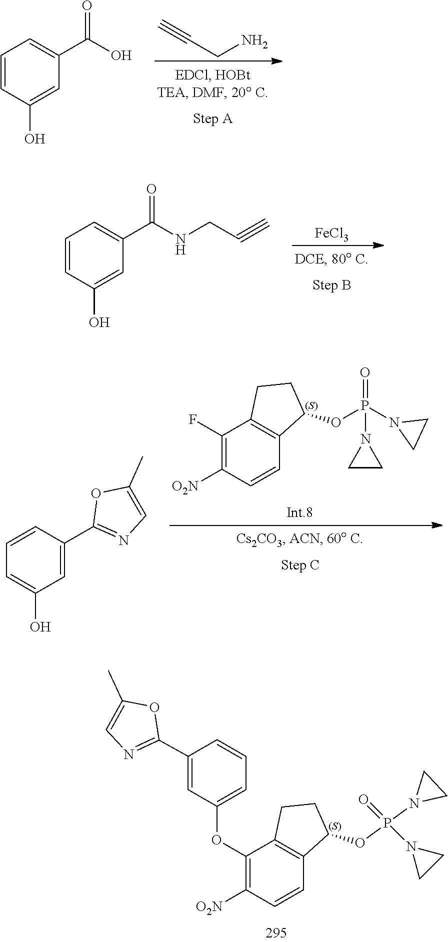

### STEP A: 3-hydroxy-N-(prop-2-yn-1-yl)benzamide

To a solution of 3-hydroxybenzoic acid (1.0 g, 7.24 mmol, 1.0 eq.), EDCI (1.67 g, 8.69 mmol, 1.2 eq.) and HOBt (1.17 g, 8.69 mmol, 1.2 eq.) in DMF (12 mL) were added prop-2-yn-1-amine (438 mg, 7.96 mmol, 1.1 eq.) and TEA (2.0 mL, 14.5 mmol, 2.0 eq.). The resulting mixture was stirred at 20° C. for 18 hrs. After completion, the reaction was diluted with H₂O (20 mL), extracted with EtOAc (20 mL×3). The organic layers were combined and washed with brine (10 mL), dried over anhydrous Na₂SO₄, and concentrated under reduced pressure. The residue was purified by flash column chromatography on silica gel to afford 3-hydroxy-N-(prop-2-yn-1-yl)benzamide (300 mg, 1.71 mmol, 24%) as a red oil. LCMS (ESI): m/z 176 [M+H]⁺.

### STEP B: 3-(5-methyloxazol-2-yl)phenol

To a solution of 3-hydroxy-N-(prop-2-yn-1-yl)benzamide (250 mg, 1.43 mmol, 1.0 eq.) in DCE (10 mL) was added ferric chloride (116 mg, 0.71 mmol, 0.5 eq.). The resulting mixture was stirred at 80° C. for 6 hrs. After completion, the reaction mixture was filtered and the filtrate was concentrated under reduced pressure. The residue was purified by prep-HPLC to afford 3-(5-methyloxazol-2-yl)phenol (200 mg, 1.14 mmol, 80%) as a white solid. LCMS (ESI): m/z 176 [M+H]⁺.

### STEP C: (S)-4-(3-(5-methyloxazol-2-yl)phenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (30 mg, 92 μmol, 1.0 eq.) and 3-(5-methyloxazol-2-yl)phenol (24 mg, 0.14 mmol, 1.5 eq.) in ACN (5 mL) was added Cs₂CO₃ (60 mg, 0.18 mmol, 2.0 eq.). The resulting mixture was stirred at 60° C. for 1 hr. After completion, the reaction mixture was concentrated under reduced pressure, the residue was purified by prep-HPLC to afford(S)-4-(3-(5-methyloxazol-2-yl)phenoxy)-5-nitro-2,3-dihydro-1H-inden-1-yl di(aziridin-1-yl)phosphinate 295 (15.4 mg, 32 μmol, 35%) as a yellow solid. LCMS (ESI): m/z 483.1 [M+H]⁺. Retention time: 1.271 min, 98% ee. Analytical method: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C. ¹H NMR (400 MHz, CDCl₃) δ 7.91 (d, J=8.2 Hz, 1H), 7.73 (d, J=7.8 Hz, 1H), 7.53 (d, J=8.2 Hz, 1H), 7.50-7.46 (m, 1H), 7.42-7.34 (m, 1H), 6.98-6.91 (m, 1H), 6.81 (d, J=1.0 Hz, 1H), 6.05-5.96 (m, 1H), 2.93-2.81 (m, 1H), 2.67-2.50 (m, 2H), 2.38 (d, J=0.9 Hz, 3H), 2.30-2.16 (m, 9H). ³¹P NMR (162 MHz, CDCl₃) δ 29.90 (s).

TABLE 10
Characterization of compounds (see Illustration 42-45)

| Compound | | |
|---|---|---|
| No. | Structure | LCMS; ¹H NMR (ppm); Retention time |
| 296 | 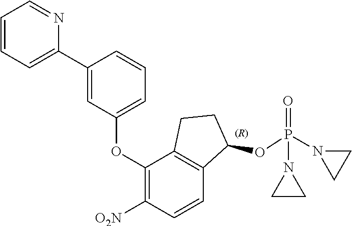 | LCMS(ESI): m/z 479.20 [M + H]⁺ ¹H NMR (400 MHz, DMSO-d₆) δ 8.63 (d, J = 4.2 Hz, 1H), 8.05 (d, J = 8.4 Hz, 1H), 7.97 (d, J = 8.0 Hz, 1H), 7.91-7.81 (m, 2H), 7.67- 7.63 (m, 1H), 7.56 (d, J = 8.2 Hz, 1H), 7.48 (t, J = 8.0 Hz, 1H), 7.40-7.33 (m, 1H), 6.99 (dd, J = 8.0, 2.6 Hz, 1H), 5.97-5.91 (m, 1H), 2.79-2.69 (m, 1H), 2.62-2.53 (m, 2H), 2.17-2.07 (m, 9H). ee: >99% Retention time: 1.006 min; Column: CHIRAL ART Cellulose-SB, 0.46 × 5 cm, 3.0 um; Mobile phase: MtBE:Hex (1:1)(0.1% DEA):MeOH = 70:30; Flow rate: 1.67 mL/min; Temperature: ambient. |
| 297 | 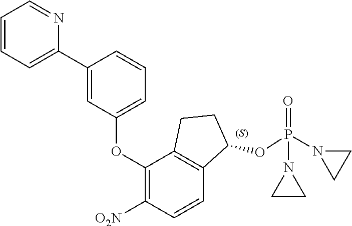 | LCMS(ESI): m/z 479.20 [M + H]⁺ ¹H NMR (400 MHz, DMSO-d₆) δ 8.64 (d, J = 4.2 Hz, 1H), 8.06 (d, J = 8.2 Hz, 1H), 7.97 (d, J = 8.0 Hz, 1H), 7.93-7.80 (m, 2H), 7.65 (t, J = 2.1 Hz, 1H), 7.56 (d, J = 8.2 Hz, 1H), 7.49 (t, J = 8.0 Hz, 1H), 7.41-7.33 (m, 1H), 6.99 (dd, J = 8.1, 2.6 Hz, 1H), 5.98- 5.91 (m, 1H), 2.79-2.71 (m, 1H), 2.62-2.53 (m, 2H), 2.17-2.06 (m, 9H). ee: >99% Retention time: 1.706 min; Column: CHIRAL ART Cellulose-SB, 0.46 × 5 cm, 3.0 um; Mobile phase: MtBE:Hex (1:1)(0.1% DEA):MeOH = 70:30; Flow rate: 1.67 mL/min; Temperature: ambient. |
| 298 | 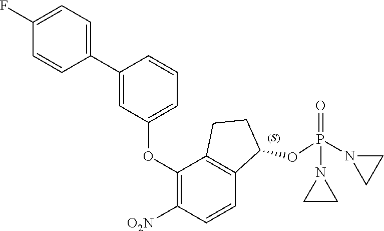 | LCMS(ESI): m/z 496.20 [M + H]⁺ ¹H NMR (400 MHz, DMSO-d₆) δ 8.04 (d, J = 8.2 Hz, 1H), 7.72- 7.63 (m, 2H), 7.55 (d, J = 8.2 Hz, 1H), 7.48-7.37 (m, 2H), 7.33 7.23 (m, 2H), 7.22-7.16 (m, 1H), 6.91-6.84 (m, 1H), 5.98-5.92 (m, 1H), 2.80-2.71 (m, 1H), 2.62-2.52 (m, 2H), 2.17-2.07 (m, 9H). ee: >99% Retention time: 1.064 min; Column: Lux 3u Cellulose-4, 0.46 × 5 cm, 3.0 um; Mobile phase: MtBE:Hex (1:1)(0.1% DEA):EtOH = 50:50; Flow rate: 1.67 mL/min; Temperature: ambient. |
| 299 | 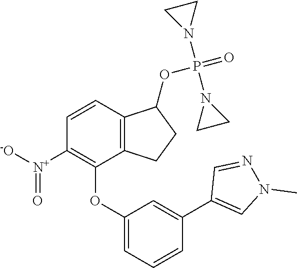 | LCMS(ESI): m/z 482.2 [M + H]⁺; ¹H NMR (400 MHz, CDCl₃) δ7.89 (d, J = 8.2 Hz, 1H), 7.70 (s, 1H), 7.57 (s, 1H), 7.51 (d, J = 8.2 Hz, 1H), 7.30-7.15 (m, 2H), 7.05- 7.00 (m, 1H), 6.65 (dd, J = 8.1, 2.4 Hz, 1H), 6.04-5.97 (m, 1H), 3.93 (s, 3H), 2.94-2.84 (m, 1H), 2.68-2.49 (m, 2H), 2.34-2.16 (m, 9H). |
| 300 | 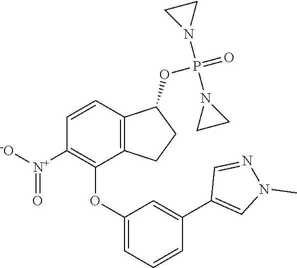 | LCMS(ESI): m/z 482.1 [M + H]⁺; ¹H NMR (400 MHz, CDCl₃) δ 7.89 (d, J = 8.2 Hz, 1H), 7.70 (s, 1H), 7.57 (s, 1H), 7.51 (d, J = 8.2 Hz, 1H), 7.29-7.26 (m, 1H), 7.19- 7.16 (m, 1H), 7.04-7.01 (m, 1H), 6.68-6.62 (m, 1H), 6.04-5.96 (m, 1H), 3.93 (s, 3H), 2.94-2.84 (m, 1H), 2.67-2.50 (m, 2H), 2.31-2.15 (m, 9H). ee: >99% Retention time: 8.646 min (Method 11). |
| 301 | 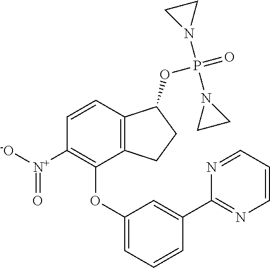 | LCMS(ESI): m/z 480.1 [M + H]⁺; ¹H NMR (400 MHz, CDCl₃) δ 8.78 (d, J = 4.8 Hz, 2H), 8.19 (d, J = 7.9 Hz, 1H), 8.01-7.96 (m, 1H), 7.92 (d, J = 8.2 Hz, 1H), 7.55-7.48 (m, 1H), 7.46-7.40 (m, 1H), 7.21-7.17 (m, 1H), 7.02-6.97 (m, 1H), 6.04-5.94 (m, 1H), 2.94-2.82 (m, 1H), 2.66-2.49 (m, 2H), 2.28-2.13 (m, 9H). ee: >99% Retention time: 3.044 min (Method 7). |
| 302 | 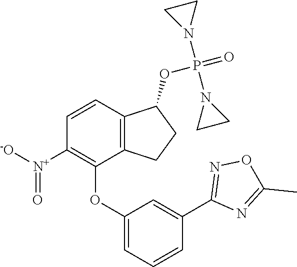 | LCMS(ESI): m/z 484.1 [M + H]⁺; ¹H NMR (400 MHz, CDCl₃) δ 7.92 (d, J = 8.2 Hz, 1H), 7.80 (d, J = 7.7 Hz, 1H), 7.60-7.49 (m, 2H), 7.43 (t, J = 8.0 Hz, 1H), 7.11-6.94 (m, 1H), 6.03-5.98 (m, 1H), 2.97-2.79 (m, 1H), 2.64 (s, 3H), 2.61- 2.51 (m, 2H), 2.31-2.17 (m, 9H). ee: >99% Retention time: 3.932 min (Method 1). |
| 303 | 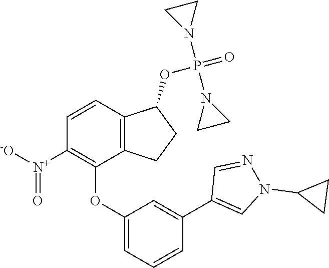 | LCMS(ESI): m/z 508.2 [M + H]⁺; ¹H NMR (400 MHz, CDCl₃) δ 7.89 (d, J = 8.2 Hz, 1H), 7.71-7.65 (m, 2H), 7.51 (d, J = 8.2 Hz, 1H), 7.26-7.23 (m, 1H), 7.20-7.16 (m, 1H), 7.04-7.01 (m, 1H), 6.68-6.62 (m, 1H), 6.04-5.96 (m, 1H), 3.66-3.58 (m, 1H), 2.93-2.83 (m, 1H), 2.67-2.48 (m, 2H), 2.29-2.16 (m, 9H), 1.18-1.13 (m, 2H), 1.07-1.02 (m, 2H). ee: >99% Retention time: 9.647 min (Method 5). |
| 304 | 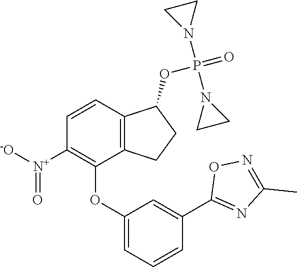 | LCMS(ESI): m/z 484.1 [M + H]⁺; ¹H NMR (400 MHz, CDCl₃) δ 7.93 (d, J = 8.2 Hz, 1H), 7.83 (d, J = 7.7 Hz, 1H), 7.59-7.54 (m, 2H), 7.47 (t, J = 8.0 Hz, 1H), 7.14-7.07 (m, 1H), 6.04-5.98 (m, 1H), 2.93-2.82 (m, 1H), 2.67-2.54 (m, 2H), 2.44 (s, 3H), 2.29-2.15 (m, 9H). ee: >99% Retention time: 3.650 min (Method 12). |
| 305 | 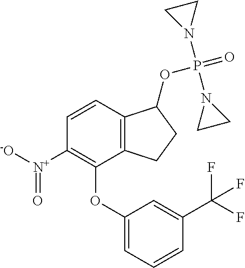 | LCMS(ESI): m/z 939.1 [2M + H]⁺; ¹H NMR (400 MHz, CDCl₃) δ 7.93 (d, J = 8.2 Hz, 1H), 7.57 (d, J = 8.2 Hz, 1H), 7.47-7.40 (m, 1H), 7.35 (d, J = 7.7 Hz, 1H), 7.14 (s, 1H), 7.04-6.98 (m, 1H), 6.07-5.95 (m, 1H), 2.91-2.80 (m, 1H), 2.66-2.51 (m, 2H), 2.33-2.14 (m, 9H). |
| 306 | 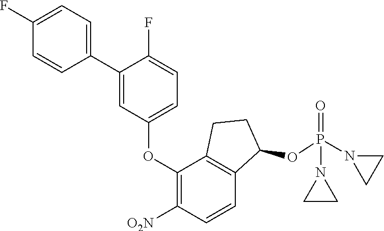 [SMILES: O=[N+]([O-])c1ccc2c(c1Oc1ccc(F)c(-c3ccc(F)cc3)c1)CC[C@H]2OP(=O)(N1CC1)N1CC1] | LCMS(ESI): m/z 514.25 [M + H]⁺ ¹H NMR (400 MHz, DMSO-d₆) δ 8.03 (d, J = 8.2 Hz, 1H), 7.61- 7.50 (m, 3H), 7.37-7.26 (m, 3H), 7.07 (dd, J = 6.3, 3.2 Hz, 1H), 6.99-6.91 (m, 1H), 5.99-5.89 (m, 1H), 2.81-2.72 (m, 1H), 2.63- 2.53 (m, 2H), 2.16-2.07 (m, 9H). ee: >99% Retention time: 1.055 min; Column: Cellulose SZ, 0.46 × 5 cm, 3.0 um; Mobile phase: MtBE:Hex (1:1)(0.1% DEA):EtOH = 70:30; Flow rate: 1.67 mL/min; Temperature: ambient. |
| 307 | 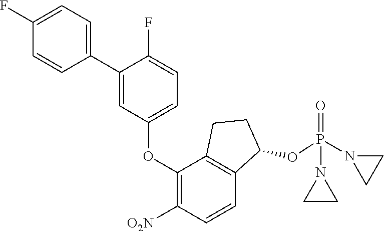 [SMILES: O=[N+]([O-])c1ccc2c(c1Oc1ccc(F)c(-c3ccc(F)cc3)c1)CC[C@@H]2OP(=O)(N1CC1)N1CC1] | LCMS(ESI): m/z 514.25 [M + H]⁺ ¹H NMR (400 MHz, DMSO-d₆) δ 8.03 (d, J = 8.2 Hz, 1H), 7.62- 7.50 (m, 3H), 7.37-7.26 (m, 3H), 7.08 (dd, J = 6.4, 3.2 Hz, 1H), 6.95 (dt, J = 9.0, 3.5 Hz, 1H), 5.99-5.89 (m, 1H), 2.80-2.72 (m, 1H), 2.63-2.53 (m, 2H), 2.19-2.04 (m, 9H). ee: >99% Retention time: 1.244 min; |
| 308 | 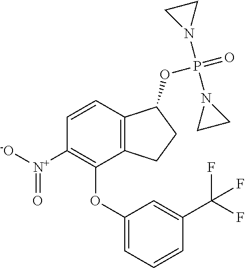 | LCMS(ESI): m/z 470.0 [M + H]⁺; ¹H NMR (400 MHz, CDCl₃) δ7.86 (d, J = 8.2 Hz, 1H), 7.50 (d, J = 8.2 Hz, 1H), 7.39-7.32 (m, 1H), 7.30-7.25 (m, 1H), 7.09-7.04 (m, 1H), 6.97-6.90 (m, 1H), 5.99-5.90 (m, 1H), 2.84-2.73 (m, 1H), 2.59-2.45 (m, 2H), 2.22-2.10 (m, 9H). ee: >99% Retention time: 4.238 min (Method 17). |
| 309 | 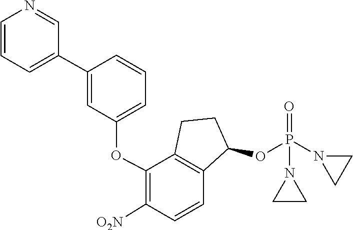 [SMILES: O=[N+]([O-])c1ccc2c(c1Oc1cccc(-c3cccnc3)c1)CC[C@H]2OP(=O)(N1CC1)N1CC1] | LCMS(ESI): m/z 479.20 [M + H]⁺ ¹H NMR (400 MHz, DMSO-d₆) δ 8.87 (d, J = 1.6 Hz, 1H), 8.58 (dd, J = 4.7, 1.6 Hz, 1H), 8.10-8.02 (m, 2H), 7.55 (d, J = 8.3 Hz, 1H), 7.52-7.44 (m, 3H), 7.33-7.28 (m, 1H), 6.97-6.89 (m, 1H), 5.99-5.92 (m, 1H), 2.80-2.72 (m, 1H), 2.63-2.53 (m, 2H), 2.18- 2.06 (m, 9H). ee: >99% Retention time: 5.901 min; Column: CHIRALPAK IE-3, 0.46 × 5 cm, 3.0 um; Mobile phase: MtBE:Hex (1:1)(0.1% DEA):MeOH = 85:15; Flow rate: 1.0 mL/min; Temperature: ambient. |
| 310 | 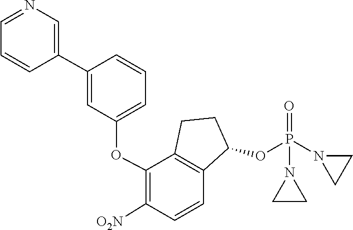 [SMILES: O=[N+]([O-])c1ccc2c(c1Oc1cccc(-c3cccnc3)c1)CC[C@@H]2OP(=O)(N1CC1)N1CC1] | LCMS(ESI): m/z 479.20 [M + H]⁺ ¹H NMR (300 MHz, DMSO-d₆) δ 8.87 (dd, J = 2.4, 0.9 Hz, 1H), 8.59 (dd, J = 4.8, 1.6 Hz, 1H), 8.10-8.02 (m, 2H), 7.56 (d, J = 8.2 Hz, 1H), 7.52-7.45 (m, 3H), 7.31 (dd, J = 2.7, 1.3 Hz, 1H), 6.97- 6.89 (m, 1H), 6.00-5.91 (m, 1H), 2.82-2.70 (m, 1H), 2.65-2.54 (m, 2H), 2.19-2.07 (m, 9H). ee: >97% Retention time: 6.755 min; (method same as 309) |
| 311 | 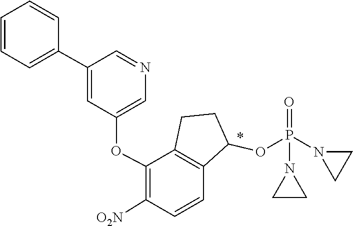 [SMILES: O=[N+]([O-])c1ccc2c(c1Oc1cncc(-c3ccccc3)c1)CCC2OP(=O)(N1CC1)N1CC1] | LCMS(ESI): m/z 479.20 [M + H]⁺ ¹H NMR (400 MHz, DMSO-d₆) δ 8.68 (d, J = 1.8 Hz, 1H), 8.30 (d, J = 2.7 Hz, 1H), 8.09 (d, J = 8.2 Hz, 1H), 7.73-7.69 (m, 2H), 7.61- 7.57 (m, 2H), 7.51-7.43 (m, 3H), 6.00-5.93 (m, 1H), 2.84- 2.76 (m, 1H), 2.66-2.53 (m, 2H), 2.18-2.07 (m, 9H). ee: >99% Retention time: 0.720 min; Column: Cellulose SB, 0.46 × 5 cm, 3.0 um; Mobile phase: MtBE:Hex (1:1)(0.1% DEA):MeOH = 50:50; Flow rate: 1.67 mL/min; Temperature: ambient. |
| 312 | 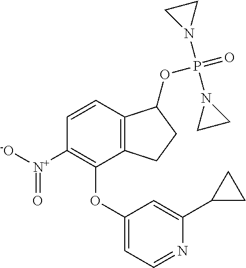 | LCMS(ESI): m/z 443.1 [M + H]⁺; ¹H NMR (400 MHz, CDCl₃) δ 8.31 (d, J = 5.7 Hz, 1H), 7.97 (d, J = 8.2 Hz, 1H), 7.60 (d, J = 8.2 Hz, 1H), 6.65 (d, J = 2.4 Hz, 1H), 6.54-6.49 (m, 1H), 6.09-5.98 (m, 1H), 2.98-2.88 (m, 1H), 2.72-2.58 (m, 2H), 2.30-2.18 (m, 9H), 2.02-1.96 (m, 1H), 1.06-0.99 (m, 4H). |
| 313 | 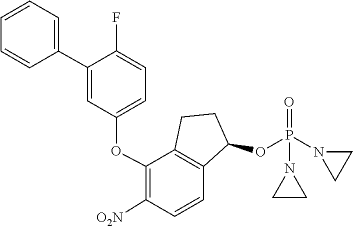 [SMILES: O=[N+]([O-])c1ccc2c(c1Oc1ccc(F)c(-c3ccccc3)c1)CC[C@H]2OP(=O)(N1CC1)N1CC1] | LCMS(ESI): m/z 496.20 [M + H]⁺ ¹H NMR (400 MHz, DMSO-d₆) δ 8.03 (d, J = 8.2 Hz, 1H), 7.56- 7.39 (m, 6H), 7.32 (dd, J = 10.2, 9.0 Hz, 1H), 7.06 (dd, J = 6.3, 3.2 Hz, 1H), 6.98-6.93 (m, 1H), 5.97-5.91 (m, 1H), 2.82-2.72 (m, 1H), 2.64-2.53 (m, 2H), 2.17-2.05 (m, 9H). ee: >99% Retention time: 1.086 min; Column: Cellulose SZ, 0.46 × 10 cm, 3.0 um; Mobile phase: Hex (0.1% DEA):EtOH = 70:30; Flow rate: 1.67 mL/min; Temperature: ambient. |
| 314 | 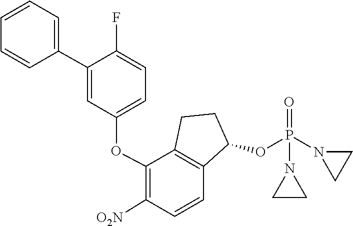 [SMILES: O=[N+]([O-])c1ccc2c(c1Oc1ccc(F)c(-c3ccccc3)c1)CC[C@@H]2OP(=O)(N1CC1)N1CC1] | LCMS(ESI): m/z 496.20 [M + H]⁺ ¹H NMR (400 MHz, DMSO-d₆) δ 8.03 (d, J = 8.2 Hz, 1H), 7.56- 7.39 (m, 6H), 7.32 (t, J = 9.6 Hz, 1H), 7.06 (dd, J = 6.3, 3.2 Hz, 1H), 6.98-6.93 (m, 1H), 5.97-5.91 (m, 1H), 2.81-2.73 (m, 1H), 2.64-2.53 (m, 2H), 2.17-2.06 (m, 9H). ee: >99% Retention time: 1.295 min; (Method same as 313) |
| 315 | 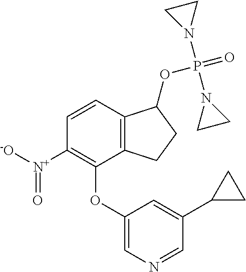 | LCMS(ESI): m/z 443.1 [M + H]⁺; ¹H NMR (400 MHz, CDCl₃) δ 8.12 (d, J = 1.3 Hz, 1H), 7.95 (d, J = 2.7 Hz, 1H), 7.89 (d, J = 8.2 Hz, 1H), 7.53 (d, J = 8.2 Hz, 1H), 6.85-6.79 (m, 1H), 6.03-5.93 (m, 1H), 2.89-2.80 (m, 1H), 2.63-2.51 (m, 2H), 2.26-2.16 (m, 9H), 1.90-1.80 (m, 1H), 1.04-0.98 (m, 2H), 0.71-0.66 (m, 2H). |
| 316 | 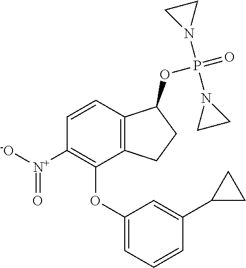 | LCMS(ESI): m/z 442.2 [M + H]⁺; ¹H NMR (400 MHz, CDCl₃) δ 7.87 (d, J = 8.2 Hz, 1H), 7.49 (d, J = 8.2 Hz, 1H), 7.14 (t, J = 8.0 Hz, 1H), 6.76 (d, J = 7.7 Hz, 1H), 6.66- 6.59 (m, 1H), 6.59-6.52 (m, 1H), 6.04-5.96 (m, 1H), 2.92-2.74 (m, 1H), 2.61-2.47 (m, 2H), 2.32-2.12 (m, 9H), 1.89-1.79 (m, 1H), 1.03-0.91 (m, 2H), 0.70-0.64 (m, 2H). ee: >99% Retention time: 2.066 min (Method 1). |
| 317 | 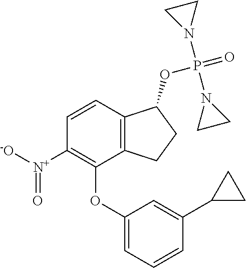 | LCMS(ESI): m/z 442.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 7.9 Hz, 1H), 6.76 (d, J = 7.7 Hz, 1H), 6.68- 6.60 (m, 1H), 6.57 (dd, J = 8.1, 2.2 Hz, 1H), 6.04-5.92 (m, 1H), 2.90-2.75 (m, 1H), 2.63-2.47 (m, 2H), 2.30-2.14 (m, 9H), 1.90-1.82 (m, 1H), 1.03-0.90 (m, 2H), 0.75-0.62 (m, 2H). ee: >99% Retention time: 2.396 min (Method 1). |
| 318 | 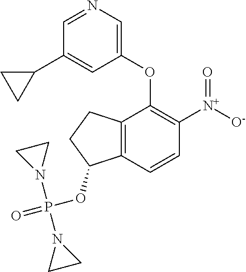 [SMILES: O=[N+]([O-])c1ccc2c(c1Oc1cncc(C3CC3)c1)CC[C@H]2OP(=O)(N1CC1)N1CC1] | LCMS(ESI): m/z 443.1 [M + H]⁺; ¹H NMR (400 MHz, CDCl₃) δ 8.15 (s, 1H), 7.98 (d, J = 2.6 Hz, 1H), 7.92 (d, J = 8.2 Hz, 1H), 7.56 (d, J = 8.2 Hz, 1H), 6.85 (s, 1H), 6.06-5.97 (m, 1H), 2.92-2.81 (m, 1H), 2.66-2.53 (m, 2H), 2.30-2.19 (m, 9H), 1.91-1.85 (m, 1H), 1.08-1.02 (m, 2H), 0.74-0.70 (m, 2H). ee: >99% Retention time: 1.236 min (Method 7). |
| 319 | 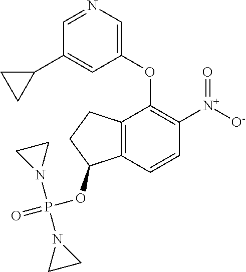 [SMILES: O=[N+]([O-])c1ccc2c(c1Oc1cncc(C3CC3)c1)CC[C@@H]2OP(=O)(N1CC1)N1CC1] | LCMS(ESI): m/z 443.1 [M + H]⁺; ¹H NMR (400 MHz, CDCl₃) δ 8.15 (s, 1H), 7.98 (d, J = 2.5 Hz, 1H), 7.92 (d, J = 8.1 Hz, 1H), 7.56 (d, J = 8.2 Hz, 1H), 6.85 (s, 1H), 6.06-5.97 (m, 1H), 2.93-2.81 (m, 1H), 2.67-2.53 (m, 2H), 2.30-2.18 (m, 9H), 1.92-1.84 (m, 1H), 1.09-1.01 (m, 2H), 0.76-0.68 (m, 2H). ee: >99% Retention time: 1.678 min (Method 7). |
| 320 | 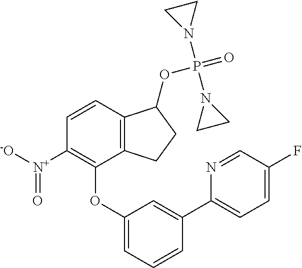 | LCMS(ESI): m/z 497.2 [M + H]⁺; ¹H NMR (400 MHz, CDCl₃) δ 8.51 (d, J = 2.8 Hz, 1H), 7.91 (d, J = 8.2 Hz, 1H), 7.71-7.66 (m, 1H), 7.64 (d, J = 7.8 Hz, 1H), 7.55-7.50 (m, 2H), 7.49-7.43 (m, 1H), 7.39 (t, J = 8.0 Hz, 1H), 6.90-6.85 (m, 1H), 6.05-5.96 (m, 1H), 2.94-2.85 (m, 1H), 2.68-2.50 (m, 2H), 2.32-2.12 (m, 9H). |
| 321 | 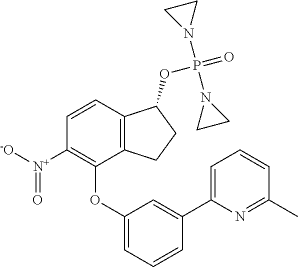 | LCMS(ESI): m/z 493.2 [M + H]⁺; ¹H NMR (400 MHz, CDCl₃) δ 7.90 (d, J = 8.2 Hz, 1H), 7.71 (d, J = 8.0 Hz, 1H), 7.63 (t, J = 7.7 Hz, 1H), 7.59-7.56 (m, 1H), 7.51 (d, J = 8.2 Hz, 1H), 7.46 (d, J = 7.8 Hz, 1H), 7.37 (t, J = 8.0 Hz, 1H), 7.10 (d, J = 7.6 Hz, 1H), 6.86-6.81 (m, 1H), 6.04-5.96 (m, 1H), 2.93-2.83 (m, 1H), 2.66-2.50 (m, 5H), 2.28-2.16 (m, 9H).ee: >99% Retention time: 3.608 min (Method 1). |
| 322 | 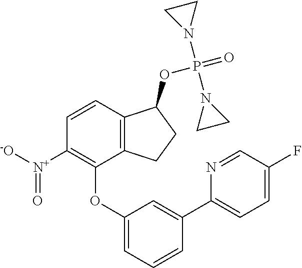 | LCMS(ESI): m/z 497.1 [M + H]⁺; ¹H NMR (400 MHz, CDCl₃) δ 8.51 (d, J = 2.8 Hz, 1H), 7.91 (d, J = 8.0 Hz, 1H), 7.71-7.66 (m, 1H), 7.64 (d, J = 7.8 Hz, 1H), 7.54-7.51 (m, 2H), 7.49-7.43 (m, 1H), 7.39 (t, J = 8.0 Hz, 1H), 6.90-6.85 (m, 1H), 6.05-5.96 (m, 1H), 2.95-2.83 (m, 1H), 2.68-2.50 (m, 2H), 2.29-2.14 (m, 9H). ee: >99% Retention time: 1.601 min (Method 15). |
| 323 | 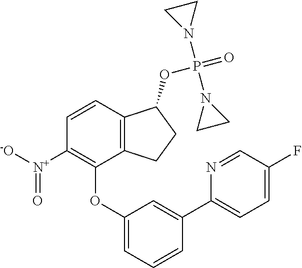 | LCMS(ESI): m/z 497.1 [M + H]⁺; ¹H NMR (400 MHz, CDCl₃) δ 8.51 (d, J = 2.8 Hz, 1H), 7.91 (d, J = 8.3 Hz, 1H), 7.71-7.66 (m, 1H), 7.64 (d, J = 7.8 Hz, 1H), 7.55-7.50 (m, 2H), 7.49-7.43 (m, 1H), 7.39 (t, J = 8.0 Hz, 1H), 6.90-6.84 (m, 1H), 6.05-5.96 (m, 1H), 2.95-2.84 (m, 1H), 2.69-2.49 (m, 2H), 2.29-2.15 (m, 9H). ee: >99% Retention time: 1.812 min (Method 15). |
| 324 | 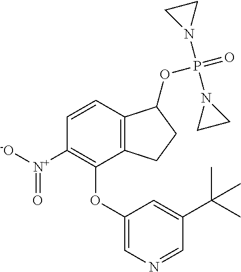 | LCMS(ESI): m/z 459.1 [M + H]⁺; ¹H NMR (400 MHz, CDCl₃) δ 8.42 (s, 1H), 7.99 (s, 1H), 7.93 (d, J = 8.2 Hz, 1H), 7.56 (d, J = 8.2 Hz, 1H), 7.27 (d, J = 6.5 Hz, 1H), 6.05-5.97 (m, 1H), 2.91-2.82 (m, 1H), 2.65-2.55 (m, 2H), 2.29-2.20 (m, 9H), 1.34 (s, 9H). |
| 325 | 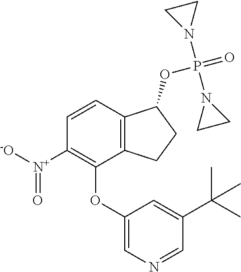 | LCMS(ESI): m/z 459.1 [M + H]⁺; ¹H NMR (400 MHz, CDCl₃) δ 8.41 (s, 1H), 8.03-7.85 (m, 2H), 7.56 (d, J = 8.1 Hz, 1H), 7.26 (d, J = 7.8 Hz, 1H), 6.05-5.95 (m, 1H), 2.93-2.76 (m, 1H), 2.66-2.49 (m, 2H), 2.29-2.16 (m, 9H), 1.33 (s, 9H). ee: >99% Retention time: 1.146 min (Method 7). |
| 326 | 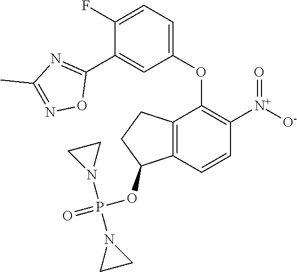 [SMILES: Cc1noc(-c2cc(Oc3c([N+](=O)[O-])ccc4c3CC[C@@H]4OP(=O)(N3CC3)N3CC3)ccc2F)n1] | LCMS(ESI): m/z 502.1 [M + H]⁺; ¹H NMR (400 MHz, CDCl₃) δ 7.94 (d, J = 8.2 Hz, 1H), 7.58 (d, J = 8.2 Hz, 1H), 7.55-7.51 (m, 1H), 7.24 (t, J = 9.3 Hz, 1H), 7.15-7.08 (m, 1H), 6.07-5.99 (m, 1H), 2.96-2.83 (m, 1H), 2.70-2.55 (m, 2H), 2.48 (s, 3H), 2.32-2.18 (m, 9H). ee: >99% Retention time: 3.565 min (Method 1). |
| 327 | 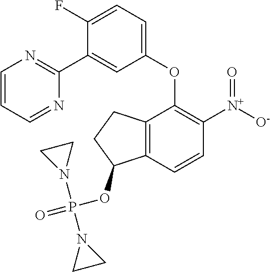 [SMILES: O=[N+]([O-])c1ccc2c(c1Oc1ccc(F)c(-c3ncccn3)c1)CC[C@@H]2OP(=O)(N1CC1)N1CC1] | LCMS(ESI): m/z 498.1 [M + H]⁺; ¹H NMR (400 MHz, CDCl₃) δ 8.85 (d, J = 4.9 Hz, 2H), 7.90 (d, J = 8.2 Hz, 1H), 7.66-7.60 (m, 1H), 7.52 (d, J = 8.2 Hz, 1H), 7.25 (d, J = 4.9 Hz, 1H), 7.19-7.12 (m, 1H), 6.99-6.93 (m, 1H), 6.04-5.95 (m, 1H), 2.93-2.85 (m, 1H), 2.68-2.52 (m, 2H), 2.27-2.17 (m, 9H). ee: >99% Retention time: 1.813 min (Method 5). |
| 328 | 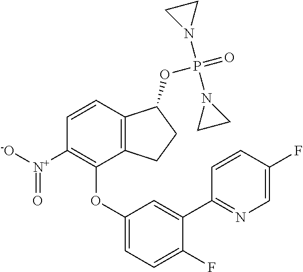 | LCMS(ESI): m/z 515.1 [M + H]⁺; ¹H NMR (400 MHz, CDCl₃) δ 8.52 (d, J = 2.8 Hz, 1H), 7.90 (d, J = 8.2 Hz, 1H), 7.83 (dd, J = 8.3, 4.1 Hz, 1H), 7.55-7.43 (m, 3H), 7.14-7.05 (m, 1H), 6.89-6.82 (m, 1H), 6.01 (dd, J = 12.3, 6.9 Hz, 1H), 2.95-2.86 (m, 1H), 2.70-2.50 (m, 2H), 2.28-2.17 (m, 9H). ee: 95% Retention time: 4.411 min (Method 39). |
| 329 | 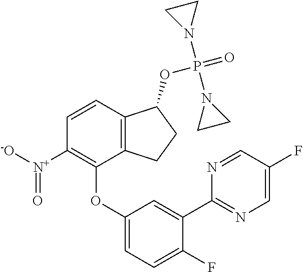 | LCMS(ESI): m/z 516.1[M + H]⁺; ¹H NMR (400 MHz, CDCl₃) δ 8.70 (s, 2H), 7.90 (d, J = 8.2 Hz, 1H), 7.63-7.45 (m, 2H), 7.16 (t, J = 9.6 Hz, 1H), 7.03-6.87 (m, 1H), 6.08-5.90 (m, 1H), 2.97-2.76 (m, 1H), 2.74-2.48 (m, 2H), 2.35-2.10 (m, 9H). ee: >99% Retention time: 2.917 min (Method 38). |
| 330 | 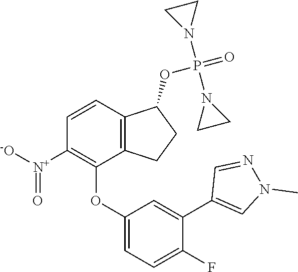 | LCMS(ESI): m/z 500.1 [M + H]⁺; ¹H NMR (400 MHz, CDCl₃) δ 7.88 (d, J = 8.2 Hz, 1H), 7.79-7.72 (m, 2H), 7.52 (d, J = 8.2 Hz, 1H), 7.12-7.07 (m, 1H), 7.02 (t, J = 9.6 Hz, 1H), 6.64-6.57 (m, 1H), 6.03-5.96 (m, 1H), 3.95 (s, 3H), 2.94-2.81 (m, 1H), 2.67-2.49 (m, 2H), 2.30-2.15 (m, 9H). ee: >99% Retention time: 5.732 min (Method 15). |
| 331 | 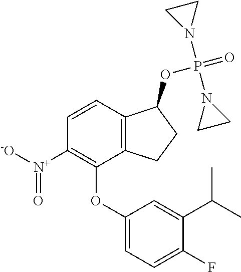 | LCMS(ESI): m/z 462.2 [M + H]⁺; ¹H NMR (400 MHz, CDCl₃) δ 7.85 (d, J = 8.1 Hz, 1H), 7.49 (d, J = 8.2 Hz, 1H), 6.93-6.87 (m, 1H), 6.86-6.80 (m, 1H), 6.59-6.51 (m, 1H), 6.03-5.94 (m, 1H), 3.24-3.15 (m, 1H), 2.86-2.76 (m, 1H), 2.60-2.50 (m, 2H), 2.27-2.17 (m, 9H), 1.22 (d, J = 6.9 Hz, 6H). ee: 82% Retention time: 3.249 min (Method 45). |
| 332 | 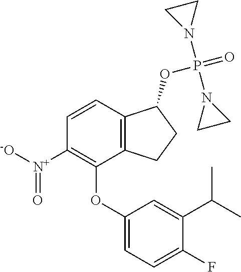 | LCMS(ESI): m/z 462.2 [M + H]⁺; ¹H NMR (400 MHz, CDCl₃) δ 7.86 (d, J = 8.2 Hz, 1H), 7.49 (d, J = 8.2 Hz, 1H), 6.92-6.87 (m, 1H), 6.85-6.81 (m, 1H), 6.58-6.53 (m, 1H), 6.03-5.95 (m, 1H), 3.25-3.14 (m, 1H), 2.88-2.75 (m, 1H), 2.61-2.50 (m, 2H), 2.29-2.15 (m, 9H), 1.26-1.18 (m, 6H). ee: 94% Retention time: 3.629 min (Method 45). |
| 333 | 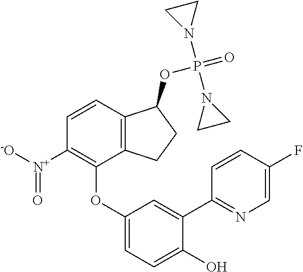 | LCMS(ESI): m/z 513.1 [M + H]⁺; ¹H NMR (400 MHz, DMSO) δ 8.66 (d, J = 2.9 Hz, 1H), 8.29-8.21 (m, 1H), 7.99 (d, J = 8.2 Hz, 1H), 7.95-7.86 (m, 1H), 7.59 (d, J = 2.9 Hz, 1H), 7.49 (d, J = 8.2 Hz, 1H), 6.94 (d, J = 8.9 Hz, 1H), 6.91-6.86 (m, 1H), 5.95-5.87 (m, 1H), 2.75-2.65 (m, 1H), 2.57-2.51 (m, 2H), 2.19-2.05 (m, 9H). ee: 99% Retention time: 5.318 min (Method 13). |
| 334 | 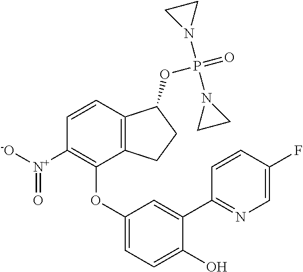 | LCMS(ESI): m/z 513.1 [M + H]⁺; ¹H NMR (400 MHz, CDCl₃) δ 13.15 (s, 1H), 8.41 (d, J = 2.8 Hz, 1H), 7.85 (d, J = 8.2 Hz, 1H), 7.82-7.73 (m, 1H), 7.63-7.54 (m, 1H), 7.48 (d, J = 8.2 Hz, 1H), 7.35 (d, J = 2.9 Hz, 1H), 7.01-6.92 (m, 1H), 6.87-6.78 (m, 1H), 6.04-5.94 (m, 1H), 2.92-2.76 (m, 1H), 2.65-2.46 (m, 2H), 2.31-2.09 (m, 9H). ee: >99% Retention time: 4.871 min (Method 13). |
| 335 | 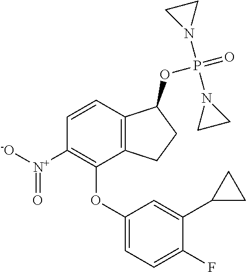 | LCMS(ESI): m/z 460.1 [M + H]⁺; ¹H NMR (400 MHz, CDCl₃) δ 7.85 (d, J = 8.2 Hz, 1H), 7.49 (d, J = 8.2 Hz, 1H), 6.93-6.85 (m, 1H), 6.54-6.45 (m, 2H), 6.02-5.93 (m, 1H), 2.86-2.73 (m, 1H), 2.61-2.47 (m, 2H), 2.29-2.16 (m, 9H), 2.11-2.01 (m, 1H), 1.03-0.94 (m, 2H), 0.70-0.63 (m, 2H). ee: 93% Retention time: 1.932 min (Method 1). |
| 336 | 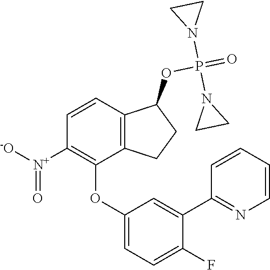 | LCMS(ESI): m/z 497.2 [M + H]⁺; ¹H NMR (400 MHz, CDCl₃) δ 8.73-8.61 (m, 1H), 7.89 (d, J = 8.2 Hz, 1H), 7.84-7.69 (m, 2H), 7.61-7.54 (m, 1H), 7.51 (d, J = 8.2 Hz, 1H), 7.28-7.23 (m, 1H), 7.16-7.04 (m, 1H), 6.92-6.78 (m, 1H), 6.07-5.92 (m, 1H), 2.99-2.82 (m, 1H), 2.73-2.48 (m, 2H), 2.34-2.12 (m, 9H). ee: >99% Retention time: 2.837 min (Method 9). |
| 337 | 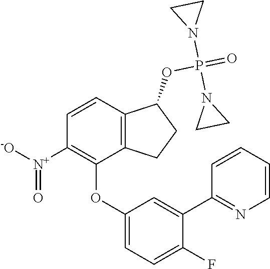 | LCMS(ESI): m/z 497.2 [M + H]⁺; ¹H NMR (400 MHz, CDCl₃) δ 8.73-8.61 (m, 1H), 7.89 (d, J = 8.2 Hz, 1H), 7.84-7.69 (m, 2H), 7.63-7.54 (m, 1H), 7.51 (d, J = 8.2 Hz, 1H), 7.32-7.21 (m, 1H), 7.18-7.04 (m, 1H), 6.96-6.74 (m, 1H), 6.12-5.85 (m, 1H), 2.99-2.81 (m, 1H), 2.78-2.43 (m, 2H), 2.34-2.12 (m, 9H). ee: 98% Retention time: 3.330 min (Method 9). |
| 338 | 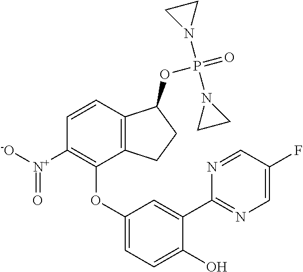 | LCMS(ESI): m/z 514.4 [M + H]⁺; ¹H NMR (400 MHz, CDCl₃) δ 12.12 (s, 1H), 8.64 (s, 2H), 7.93 (d, J = 2.6 Hz, 1H), 7.87 (d, J = 8.2 Hz, 1H), 7.48 (d, J = 8.2 Hz, 1H), 7.05-6.97 (m, 2H), 6.02-5.95 (m, 1H), 2.92-2.78 (m, 1H), 2.64-2.49 (m, 2H), 2.27-2.17 (m, 9H). ee: >99% Retention time: 3.603 min (Method 2). |
| 339 | 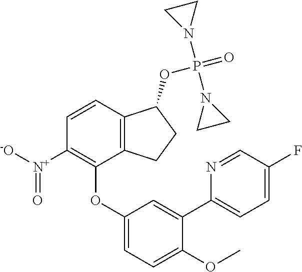 | LCMS(ESI): m/z 527.2 [M + H]⁺; ¹H NMR (400 MHz, DMSO) δ 8.61 (d, J = 3.0 Hz, 1H), 8.04-7.95 (m, 2H), 7.80-7.71 (m, 1H), 7.51 (d, J = 8.2 Hz, 1H), 7.32 (d, J = 3.2 Hz, 1H), 7.16 (d, J = 9.1 Hz, 1H), 7.01 (dd, J = 9.0, 3.2 Hz, 1H), 5.96-5.86 (m, 1H), 3.83 (s, 3H), 2.77-2.52 (m, 3H), 2.16-2.07 (m, 9H). ee: 96% Retention time: 3.775 min (Method 1). |
| 340 | 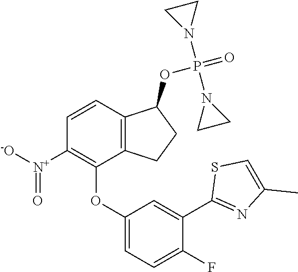 | LCMS(ESI): m/z 517.1 [M + H]⁺; ¹H NMR (400 MHz, CDCl₃) δ 7.91 (d, J = 8.1 Hz, 1H), 7.86-7.81 (m, 1H), 7.53 (d, J = 8.2 Hz, 1H), 7.12 (t, J = 9.6 Hz, 1H), 7.02 (s, 1H), 6.89-6.80 (m, 1H), 6.05-5.97 (m, 1H), 2.96-2.83 (m, 1H), 2.69-2.53 (m, 2H), 2.50 (s, 3H), 2.29-2.17 (m, 9H). ee: 95% Retention time: 1.602 min (Method 5). |
| 341 | 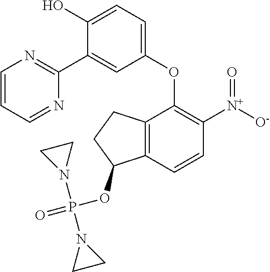 [SMILES: O=[N+]([O-])c1ccc2c(c1Oc1ccc(O)c(-c3ncccn3)c1)CC[C@@H]2OP(=O)(N1CC1)N1CC1] | LCMS(ESI): m/z 496.2 [M + H]⁺; ¹H NMR (400 MHz, CDCl₃) δ 12.84 (s, 1H), 8.76 (d, J = 4.9 Hz, 2H), 8.00 (d, J = 2.9 Hz, 1H), 7.87 (d, J = 8.2 Hz, 1H), 7.47 (d, J = 8.2 Hz, 1H), 7.23 (t, J = 4.9 Hz, 1H), 7.04-6.98 (m, 2H), 6.03-5.93 (m, 1H), 2.89-2.82 (m, 1H), 2.63-2.48 (m, 2H), 2.35-2.08 (m, 9H).. ee: >99% Retention time: 3.528 min (Method 7). |
| 342 | 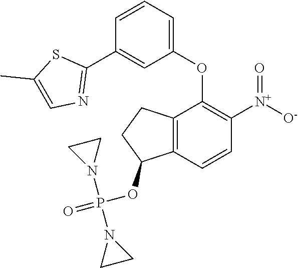 [SMILES: Cc1cnc(-c2cccc(Oc3c([N+](=O)[O-])ccc4c3CC[C@@H]4OP(=O)(N3CC3)N3CC3)c2)s1] | LCMS(ESI): m/z 499.1 [M + H]⁺; ¹H NMR (400 MHz, CDCl₃) δ 7.91 (d, J = 8.2 Hz, 1H), 7.57 (d, J = 7.8 Hz, 1H), 7.53 (d, J = 8.2 Hz, 1H), 7.49-7.43 (m, 2H), 7.37-7.32 (m, 1H), 6.91-6.85 (m, 1H), 6.05-5.94 (m, 1H), 2.94-2.83 (m, 1H), 2.67-2.54 (m, 2H), 2.50 (s, 3H), 2.28-2.18 (m, 9H). ee: >99% Retention time: 3.791 min (Method 7). |
| 343 | 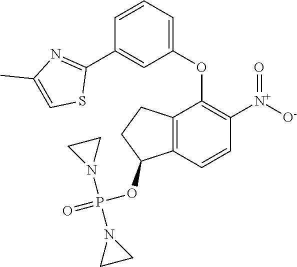 [SMILES: Cc1csc(-c2cccc(Oc3c([N+](=O)[O-])ccc4c3CC[C@@H]4OP(=O)(N3CC3)N3CC3)c2)n1] | LCMS(ESI): m/z 499.1 [M + H]⁺; ¹H NMR (400 MHz, CDCl₃) δ 7.91 (d, J = 8.2 Hz, 1H), 7.63 (d, J = 7.9 Hz, 1H), 7.56-7.51 (m, 2H), 7.34 (t, J = 8.0 Hz, 1H), 6.89 (d, 0.8 Hz, 1H), 6.86-6.81 (m, 1H), 6.04-5.97 (m, 1H), 2.92-2.84 (m, J = 1H), 2.68-2.51 (m, 2H), 2.49 (s, 3H), 2.29-2.17 (m, 9H). ee: 94% Retention time: 4.376 min (Method 1). |
| 344 | 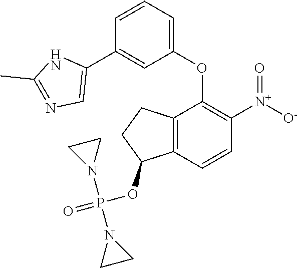 [SMILES: Cc1ncc(-c2cccc(Oc3c([N+](=O)[O-])ccc4c3CC[C@@H]4OP(=O)(N3CC3)N3CC3)c2)[nH]1] | LCMS(ESI): m/z 482.2 [M + H]⁺; ¹H NMR (400 MHz, CDCl₃) δ 7.87 (d, J = 8.2 Hz, 1H), 7.47 (d, J = 8.1 Hz, 1H), 7.37 (d, J = 7.5 Hz, 1H), 7.31 (d, J = 8.0 Hz, 1H), 7.18 (s, 1H), 7.11 (s, 1H), 6.80 (d, J = 7.0 Hz, 1H), 5.95-5.85 (m, 1H), 2.97-2.89 (m, 1H), 2.68-2.62 (m, 1H), 2.53-2.48 (m, 1H), 2.44 (s, 3H), 2.24-2.15 (m, 9H). ee: 94% Retention time: 2.704 min (Method 1). |
| 345 | 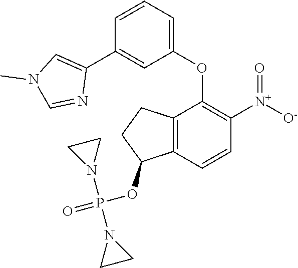 [SMILES: Cn1cnc(-c2cccc(Oc3c([N+](=O)[O-])ccc4c3CC[C@@H]4OP(=O)(N3CC3)N3CC3)c2)c1] | LCMS(ESI): m/z 482.2 [M + H]⁺; ¹H NMR (400 MHz, CDCl₃) δ 7.88 (d, J = 8.2 Hz, 1H), 7.50-7.42 (m, 3H), 7.34-7.27 (m, 2H), 7.15 (d, J = 1.1 Hz, 1H), 6.75-6.71 (m, 1H), 6.02-5.94 (m, 1H), 3.71 (s, 3H), 2.91-2.81 (m, 1H), 2.64-2.48 (m, 2H), 2.27-2.17 (m, 9H). ee: 97% Retention time: 5.525 min (Method 30). |
| 346 | 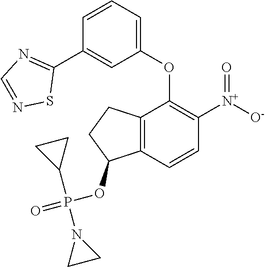 [SMILES: O=[N+]([O-])c1ccc2c(c1Oc1cccc(-c3ncns3)c1)CC[C@@H]2OP(=O)(C1CC1)N1CC1] | LCMS(ESI): m/z 486.1 [M + H]⁺; ¹H NMR (400 MHz, CDCl₃) δ 8.68 (s, 1H), 7.94 (d, J = 8.2 Hz, 1H), 7.67 (d, J = 8.0 Hz, 1H), 7.56 (dd, J = 7.7, 5.1 Hz, 2H), 7.45 (t, J = 8.0 Hz, 1H), 7.02 (dd, J = 7.9, 2.2 Hz, 1H), 6.03 (dd, J = 12.2, 6.9 Hz, 1H), 2.96-2.87 (m, 1H), 2.70-2.53 (m, 2H), 2.29-2.18 (m, 9H). ee: 96% Retention time: 2.194 min (Method 5). |
| 347 | 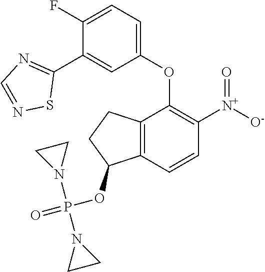 [SMILES: O=[N+]([O-])c1ccc2c(c1Oc1ccc(F)c(-c3ncns3)c1)CC[C@@H]2OP(=O)(N1CC1)N1CC1] | LCMS(ESI): m/z 504.2 [M + H]⁺; ¹H NMR (400 MHz, CDCl₃) δ 8.71 (d, J = 3.5 Hz, 1H), 7.94 (d, J = 8.2 Hz, 1H), 7.81 (dd, J = 5.4, 3.2 Hz, 1H), 7.58 (d, J = 8.2 Hz, 1H), 7.24 (d, J = 9.5 Hz, 1H), 7.12-7.05 (m, 1H), 6.03 (dd, J = 12.3, 7.0 Hz, 1H), 2.97-2.86 (m, 1H), 2.71-2.53 (m, 2H), 2.31-2.18 (m, 9H). ee: 97% Retention time: 2.067 min (Method 5). |
| 348 | 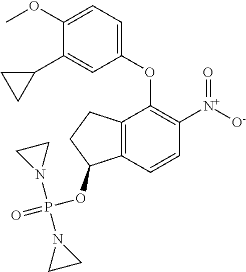 [SMILES: COc1ccc(Oc2c([N+](=O)[O-])ccc3c2CC[C@@H]3OP(=O)(N2CC2)N2CC2)cc1C1CC1] | LCMS(ESI): m/z 472.1 [M + H]⁺; ¹H NMR (400 MHz, CDCl₃) δ 7.82 (d, J = 8.2 Hz, 1H), 7.44 (d, J = 8.2 Hz, 1H), 6.71 (d, J = 8.8 Hz, 1H), 6.58-6.50 (m, 1H), 6.47 (d, J = 3.0 Hz, 1H), 6.04-5.88 (m, 1H), 3.83 (s, 3H), 2.92-2.64 (m, 1H), 2.58-2.41 (m, 2H), 2.34-2.08 (m, 10H), 0.99-0.86 (m, 2H), 0.67- 0.52 (m, 2H). ee: 88% Retention time: 2.303 min (Method 1). |
| 349 | 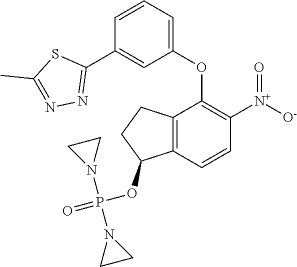 [SMILES: Cc1nnc(-c2cccc(Oc3c([N+](=O)[O-])ccc4c3CC[C@@H]4OP(=O)(N3CC3)N3CC3)c2)s1] | LCMS(ESI): m/z 500.1 [M + H]⁺; ¹H NMR (400 MHz, CDCl₃) δ 7.93 (d, J = 8.2 Hz, 1H), 7.59-7.53 (m, 2H), 7.53-7.50 (m, 1H), 7.43-7.38 (m, 1H), 7.01-6.94 (m, 1H), 6.06-5.97 (m, 1H), 2.95-2.86 (m, 1H), 2.81 (s, 3H), 2.69-2.53 (m, 2H), 2.34-2.12 (m, 9H). ee: 95% Retention time: 5.754 min (Method 9). |
| 350 | 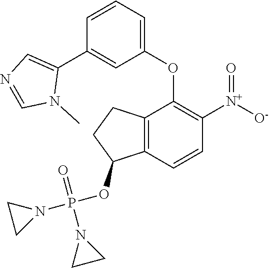 [SMILES: Cn1cncc1-c1cccc(Oc2c([N+](=O)[O-])ccc3c2CC[C@@H]3OP(=O)(N2CC2)N2CC2)c1] | LCMS(ESI): m/z 482.2 [M + H]⁺; ¹H NMR (400 MHz, CDCl₃) δ 7.90 (d, J = 8.2 Hz, 1H), 7.54 (d, J = 8.2 Hz, 1H), 7.51 (s, 1H), 7.39-7.34 (m, 1H), 7.13-7.09 (m, 1H), 7.09-7.06 (m, 1H), 6.92-6.88 (m, 1H), 6.88-6.83 (m, 1H), 6.06-5.97 (m, 1H), 3.66-3.63 (m, 3H), 2.96-2.87 (m, 1H), 2.70-2.53 (m, 2H), 2.33-2.13 (m, 9H). ee: 95% Retention time: 5.153 min (Method 30). |
| 351 | 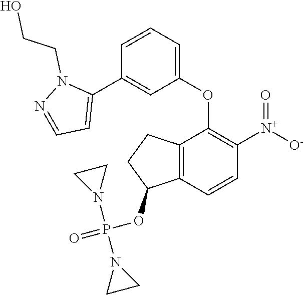 [SMILES: O=[N+]([O-])c1ccc2c(c1Oc1cccc(-c3ccnn3CCO)c1)CC[C@@H]2OP(=O)(N1CC1)N1CC1] | LCMS(EST): m/z 512.4 [M + H]⁺; ¹H NMR (400 MHz, CDCl₃) δ 7.90 (d, J = 8.2 Hz, 1H), 7.57-7.49 (m, 2H), 7.39 (t, J = 7.9 Hz, 1H), 7.12 (d, J = 7.6 Hz, 1H), 6.94 (d, J = 7.4 Hz, 2H), 6.30 (s, 1H), 6.00 (dd, J = 11.9, 6.8 Hz, 1H), 4.23- 4.11 (m, 2H), 4.00-3.91 (m, 2H), 3.54 (s, 1H), 3.01-2.85 (m, 1H), 2.77-2.49 (m, 2H), 2.35-2.13 (m, 9H). ee: 96% Retention time: 2.900 min (Method 1). |
| 352 | 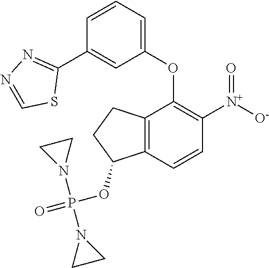 [SMILES: O=[N+]([O-])c1ccc2c(c1Oc1cccc(-c3nncs3)c1)CC[C@H]2OP(=O)(N1CC1)N1CC1] | LCMS(ESI): m/z 486.1 [M + H]⁺; ¹H NMR (400 MHz, CDCl₃) δ9.12 (s, 1H), 7.94 (d, J = 8.2 Hz, 1H), 7.64 (d, J = 7.8 Hz, 1H), 7.61-7.54 (m, 2H), 7.47-7.40 (m, 1H), 7.05-7.00 (m, 1H), 6.06-5.99 (m, 1H), 2.96-2.87 (m, 1H), 2.71-2.54 (m, 2H), 2.29-2.19 (m, 9H). ee: 98% Retention time: 4.469 min (Method 3). |
| 353 | 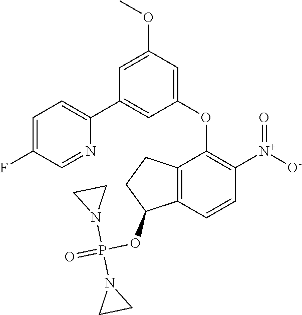 [SMILES: COc1cc(Oc2c([N+](=O)[O-])ccc3c2CC[C@@H]3OP(=O)(N2CC2)N2CC2)cc(-c2ccc(F)cn2)c1] | LCMS(ESI): m/z 527.1 [M + H]⁺; ¹H NMR (400 MHz, CDCl₃) δ 8.49 (d, J = 2.0 Hz, 1H), 7.91 (d, J = 8.2 Hz, 1H), 7.64 (dd, J = 8.7, 4.2 Hz, 1H), 7.53 (d, J = 8.2 Hz, 1H), 7.45 (td, J = 8.2, 2.0 Hz, 1H), 7.25 (d, J = 8.9 Hz, 2H), 7.04 (s, 1H), 6.46 (s, 1H), 6.01 (dd, J = 12.5, 6.7 Hz, 1H), 3.86 (s, 3H), 3.02-2.84 (m, 1H), 2.77-2.46 (m, 2H), 2.35-2.10 (m, 10H). ee: >99% Retention time: 3.445 min (Method 47). |
| 354 | 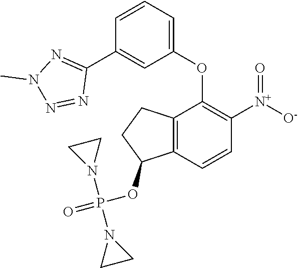 [SMILES: Cn1nnc(-c2cccc(Oc3c([N+](=O)[O-])ccc4c3CC[C@@H]4OP(=O)(N3CC3)N3CC3)c2)n1] | LCMS(ESI): m/z 484.1 [M + H]⁺; ¹H NMR (400 MHz, CDCl₃) δ 7.95-7.84 (m, 2H), 7.63 (s, 1H), 7.54 (d, J = 8.2 Hz, 1H), 7.47-7.40 (m, 1H), 7.05-6.92 (m, 1H), 6.08-5.93 (m, 1H), 4.38 (s, 3H), 2.95-2.80 (m, 1H), 2.70-2.47 (m, 2H), 2.29-2.17 (m, 9H). ee: 97% Retention time: 4.957 min (Method 1). |
| 355 | 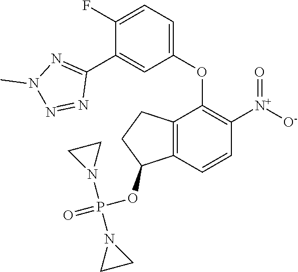 [SMILES: Cn1nnc(-c2cc(Oc3c([N+](=O)[O-])ccc4c3CC[C@@H]4OP(=O)(N3CC3)N3CC3)ccc2F)n1] | LCMS(ESI): m/z 502.1 [M + H]⁺; ¹H NMR (400 MHz, CDCl₃) δ 7.92 (d, J = 8.2 Hz, 1H), 7.68-7.58 (m, 1H), 7.55 (d, J = 8.2 Hz, 1H), 7.24-7.16 (m, 1H), 7.05-6.96 (m, 1H), 6.08-5.95 (m, 1H), 4.41 (d, J = 17.6 Hz, 3H), 2.95-2.84 (m, 1H), 2.69-2.51 (m, 2H), 2.31-2.12 (m, 9H). ee: 97% Retention time: 4.361 min (Method 1). |
| 356 | 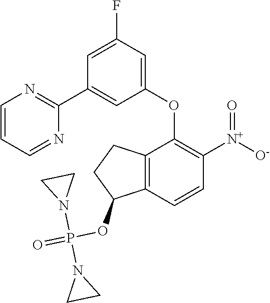 [SMILES: O=[N+]([O-])c1ccc2c(c1Oc1cc(F)cc(-c3ncccn3)c1)CC[C@@H]2OP(=O)(N1CC1)N1CC1] | LCMS(ESI): m/z 498.1 [M + H]⁺; ¹H NMR (400 MHz, CDCl₃) δ 8.85-8.74 (m, 2H), 8.00-7.89 (m, 2H), 7.86-7.74 (m, 1H), 7.64-7.52 (m, 1H), 7.24-7.19 (m, 1H), 6.92-6.58 (m, 1H), 6.15-5.93 (m, 1H), 2.98-2.86 (m, 1H), 2.72- 2.52 (m, 2H), 2.35-2.13 (m, 9H). ee: >99% Retention time: 2.079 min (Method 50). |
| 357 | 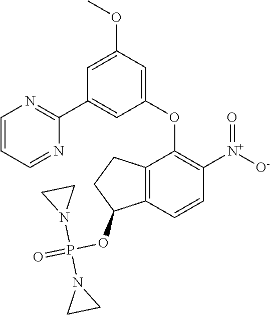 [SMILES: COc1cc(Oc2c([N+](=O)[O-])ccc3c2CC[C@@H]3OP(=O)(N2CC2)N2CC2)cc(-c2ncccn2)c1] | LCMS(ESI): m/z 510.1 [M + H]⁺; ¹H NMR (400 MHz, CDCl₃) δ 8.77 (d, J = 4.8 Hz, 2H), 7.92 (d, J = 8.2 Hz, 1H), 7.78 (dd, J = 2.1, 1.3 Hz, 1H), 7.59 (dd, J = 2.2, 1.2 Hz, 1H), 7.52 (d, J = 8.2 Hz, 1H), 7.19 (t, J = 4.8 Hz, 1H), 6.58 (t, J = 2.4 Hz, 1H), 6.03-5.98 (m, 1H), 3.89 (s, 3H), 2.96-2.88 (m, 1H), 2.71-2.51 (m, 2H), 2.29-2.17 (m, 9H). ee: 96% Retention time: 5.374 min (Method 30). |
| 358 | 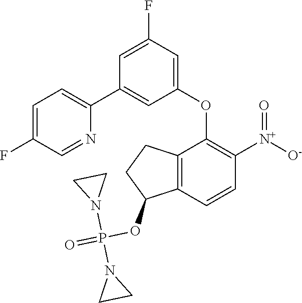 [SMILES: O=[N+]([O-])c1ccc2c(c1Oc1cc(F)cc(-c3ccc(F)cn3)c1)CC[C@@H]2OP(=O)(N1CC1)N1CC1] | LCMS(ESI): m/z 515.1 [M + H]⁺; ¹H NMR (400 MHz, CDCl₃) δ 8.51 (d, J = 2.9 Hz, 1H), 7.94 (d, J = 8.2 Hz, 1H), 7.68-7.62 (m, 1H), 7.57 (d, J = 8.2 Hz, 1H), 7.51-7.43 (m, 1H), 7.42-7.36 (m, 1H), 7.34-7.28 (m, 1H), 6.63-6.54 (m, 1H), 6.07-5.98 (m, 1H), 2.98-2.89 (m, 1H), 2.72-2.53 (m, 2H), 2.32-2.15 (m, 9H). ee: >99% Retention time: 3.324 min (Method 9). |
| 359 | 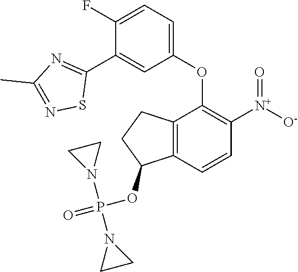 [SMILES: Cc1nsc(-c2cc(Oc3c([N+](=O)[O-])ccc4c3CC[C@@H]4OP(=O)(N3CC3)N3CC3)ccc2F)n1] | LCMS(ESI): m/z 518.1 [M + H]⁺; ¹H NMR (400 MHz, CDCl₃) δ 7.94 (d, J = 8.2 Hz, 1H), 7.83-7.78 (m, 1H), 7.57 (d, J = 8.2 Hz, 1H), 7.24-7.18 (m, 1H), 7.04-6.98 (m, 1H), 6.07-5.98 (m, 1H), 2.96-2.85 (m, 1H), 2.71 (s, 3H), 2.69-2.54 (m, 2H), 2.34-2.12 (m, 9H). ee: 96% Retention time: 2.358 min (Method 51). |
| 360 | 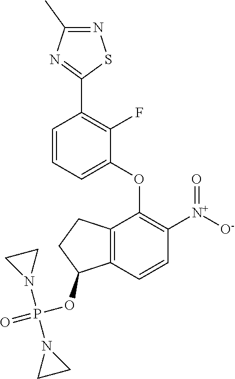 [SMILES: Cc1nsc(-c2cccc(Oc3c([N+](=O)[O-])ccc4c3CC[C@@H]4OP(=O)(N3CC3)N3CC3)c2F)n1] | LCMS(ESI): m/z 518.1 [M + H]⁺; ¹H NMR (400 MHz, CDCl₃) δ 8.06-7.99 (m, 1H), 7.96 (d, J = 8.2 Hz, 1H), 7.58 (d, J = 8.2 Hz, 1H), 7.20-7.15 (m, 1H), 6.89-6.83 (m, 1H), 6.06-5.98 (m, 1H), 2.97-2.89 (m, 1H), 2.77 (s, 3H), 2.72-2.55 (m, 2H), 2.31-2.17 (m, 9H). ee: >99% Retention time: 2.569 min (Method 51). |
| 361 | 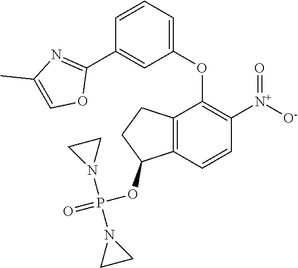 [SMILES: Cc1coc(-c2cccc(Oc3c([N+](=O)[O-])ccc4c3CC[C@@H]4OP(=O)(N3CC3)N3CC3)c2)n1] | LCMS(ESI): m/z 483.1 [M + H]⁺; ¹H NMR (400 MHz, CDCl₃) δ 7.91 (d, J = 8.2 Hz, 1H), 7.75 (d, J = 7.8 Hz, 1H), 7.58-7.49 (m, 2H), 7.45-7.33 (m, 2H), 6.95 (dd, J = 8.1, 2.2 Hz, 1H), 6.01 (t, J = 6.2 Hz, 1H), 2.99-2.77 (m, 1H), 2.71- 2.50 (m, 2H), 2.37-2.09 (m, 13H). ee: >99% Retention time: 1.871 min (Method 7). |
| 362 | 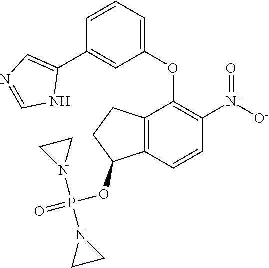 [SMILES: O=[N+]([O-])c1ccc2c(c1Oc1cccc(-c3cnc[nH]3)c1)CC[C@@H]2OP(=O)(N1CC1)N1CC1] | LCMS(ESI): m/z 468.1 [M + H]⁺; ¹H NMR (400 MHz, CDCl₃) δ 7.87 (d, J = 8.2 Hz, 1H), 7.70 (s, 1H), 7.48 (d, J = 8.2 Hz, 1H), 7.42 (d, J = 7.8 Hz, 1H), 7.36-7.29 (m, 2H), 7.18 (s, 1H), 6.88-6.79 (m, 1H), 6.06-5.89 (m, 1H), 3.01- 2.86 (m, 1H), 2.73-2.60 (m, 1H), 2.57-2.46 (m, 1H), 2.30-2.15 (m, 9H). ee: >99% Retention time: 4.701 min (Method 27). |
| 363 | 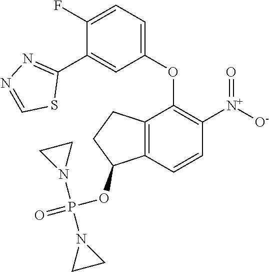 [SMILES: O=[N+]([O-])c1ccc2c(c1Oc1ccc(F)c(-c3nncs3)c1)CC[C@@H]2OP(=O)(N1CC1)N1CC1] | LCMS(ESI): m/z 504.1 [M + H]⁺; ¹H NMR (400 MHz, CDCl₃) δ 9.24 (s, 1H), 7.94 (d, J = 8.2 Hz, 1H), 7.89-7.81 (m, 1H), 7.57 (d, J = 8.3 Hz, 1H), 7.25-7.21 (m, 1H), 7.15-7.10 (m, 1H), 6.07-5.85 (m, 1H), 2.98-2.89 (m, 1H), 2.72-2.55 (m, 2H), 2.32-2.14 (m, 9H). ee: 96% Retention time: 6.223 min (Method 30). |
| 364 | 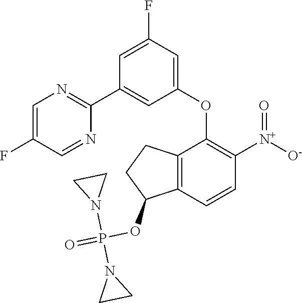 [SMILES: O=[N+]([O-])c1ccc2c(c1Oc1cc(F)cc(-c3ncc(F)cn3)c1)CC[C@@H]2OP(=O)(N1CC1)N1CC1] | LCMS(ESI): m/z 538.1 [M + Na]⁺; ¹H NMR (400 MHz, CDCl₃) δ 8.63 (s, 2H), 7.95 (d, J = 8.2 Hz, 1H), 7.86 (d, J = 9.5 Hz, 1H), 7.71 (s, 1H), 7.58 (d, J = 8.2 Hz, 1H), 6.76-6.68 (m, 1H), 6.08-5.94 (m, 1H), 2.98-2.88 (m, 1H), 2.72-2.52 (m, 2H), 2.32-2.12 (m, 9H). ee: 98% Retention time: 4.516 min (Method 1). |
| 365 | 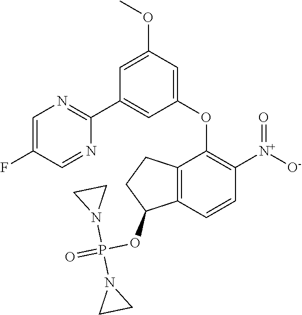 [SMILES: COc1cc(Oc2c([N+](=O)[O-])ccc3c2CC[C@@H]3OP(=O)(N2CC2)N2CC2)cc(-c2ncc(F)cn2)c1] | LCMS(ESI): m/z 528.2 [M + H]⁺; ¹H NMR (400 MHz, CDCl₃) δ 8.61 (s, 2H), 7.92 (d, J = 8.2 Hz, 1H), 7.71 (s, 1H), 7.56-7.48 (m, 2H), 6.62-6.54 (m, 1H), 6.09-5.89 (m, 1H), 3.89 (s, 3H), 2.97-2.87 (m, 1H), 2.72-2.51 (m, 2H), 2.30- 2.14 (m, 9H). ee: 92% Retention time: 3.463 min (Method 5). |
| 366 | 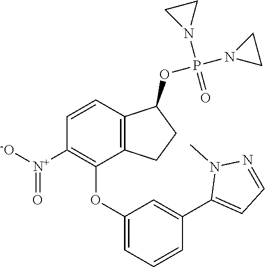 | LCMS(ESI): m/z 482.2 [M + H]⁺; ¹H NMR (400 MHz, CDCl₃) δ 7.90 (d, J = 8.2 Hz, 1H), 7.54 (d, J = 8.2 Hz, 1H), 7.49 (d, J = 1.6 Hz, 1H), 7.38 (t, J = 7.9 Hz, 1H), 7.13 (d, J = 7.6 Hz, 1H), 6.95-6.86 (m, 2H), 6.29 (d, J = 1.6 Hz, 1H), 6.01 (dd, J = 12.1, 7.0 Hz, 1H), 3.86 (s, 3H), 2.97-2.87 (m, 1H), 2.73-2.51 (m, 2H), 2.31-2.15 (m, 9H). ee: 97% Retention time: 3.290 min (Method 1). |
| 367 | 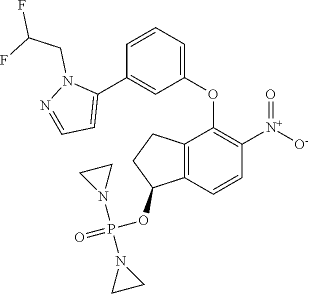 [SMILES: O=[N+]([O-])c1ccc2c(c1Oc1cccc(-c3ccnn3CC(F)F)c1)CC[C@@H]2OP(=O)(N1CC1)N1CC1] | LCMS(ESI): m/z 532.1 [M + H]⁺; ¹H NMR (400 MHz, CDCl₃) δ7.91 (d, J = 8.2 Hz, 1H), 7.58 (d, J = 1.8 Hz, 1H), 7.54 (d, J = 8.2 Hz, 1H), 7.45-7.39 (m, 1H), 7.11 (d, J = 7.7 Hz, 1H), 7.03-6.95 (m, 1H), 6.89-6.84 (m, 1H), 6.35-5.96 (m, 3H), 4.48-4.22 (m, 2H), 2.99-2.86 (m, 1H), 2.72-2.52 (m, 2H), 2.31-2.14 (m, 9H). ee: 97% Retention time: 2.138 min (Method 1). |
| 368 | 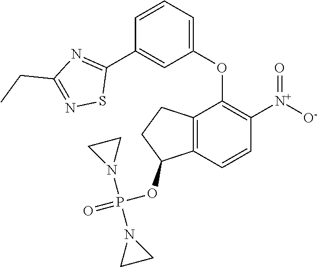 [SMILES: CCc1nsc(-c2cccc(Oc3c([N+](=O)[O-])ccc4c3CC[C@@H]4OP(=O)(N3CC3)N3CC3)c2)n1] | LCMS(ESI): m/z 514.1 [M + H]⁺; ¹H NMR (400 MHz, CDCl₃) δ 7.94 (d, J = 8.2 Hz, 1H), 7.64 (d, J = 7.7 Hz, 1H), 7.59-7.52 (m, 2H), 7.41 (t, J = 8.0 Hz, 1H), 6.98-6.93 (m, 1H), 6.06-5.97 (m, 1H), 3.09-3.00 (m, 2H), 2.95-2.84 (m, 1H), 2.70-2.51 (m, 2H), 2.32-2.16 (m, 9H), 1.43 (t, J = 7.6 Hz, 3H). ee: >99% Retention time: 2.100 min (Method 42). |
| 369 | 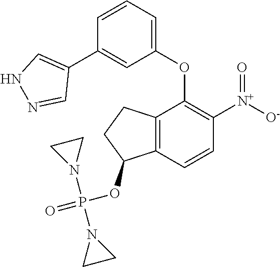 [SMILES: O=[N+]([O-])c1ccc2c(c1Oc1cccc(-c3cn[nH]c3)c1)CC[C@@H]2OP(=O)(N1CC1)N1CC1] | LCMS(ESI): m/z 468.1 [M + H]⁺; ¹H NMR (400 MHz, CDCl₃) δ 7.90 (d, J = 8.2 Hz, 1H), 7.82 (s, 2H), 7.52 (d, J = 8.2 Hz, 1H), 7.30 (d, J = 7.8 Hz, 1H), 7.23 (d, J = 7.5 Hz, 1H), 7.08 (s, 1H), 6.67 (d, J = 7.0 Hz, 1H), 6.08-5.96 (m, 1H), 2.94-2.84 (m, 1H), 2.68-2.50 (m, 2H), 2.29-2.18 (m, 9H). ee: 98% Retention time: 5.466 min (Method 27). |
| 370 | 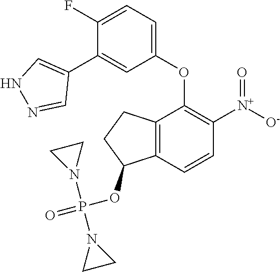 [SMILES: O=[N+]([O-])c1ccc2c(c1Oc1ccc(F)c(-c3cn[nH]c3)c1)CC[C@@H]2OP(=O)(N1CC1)N1CC1] | LCMS(ESI): m/z 486.1 [M + H]⁺; ¹H NMR (400 MHz, CDCl₃) δ 7.94 (d, J = 1.5 Hz, 2H), 7.89 (d, J = 8.2 Hz, 1H), 7.54-7.50 (m, 1H), 7.16-7.11 (m, 1H), 7.07-7.01 (m, 1H), 6.67-6.58 (m, 1H), 6.05-5.96 (m, 1H), 2.94-2.84 (m, 1H), 2.68-2.50 (m, 2H), 2.29-2.16 (m, 10H). ee: 97% Retention time: 4.765 min (Method 3). |
| 371 | 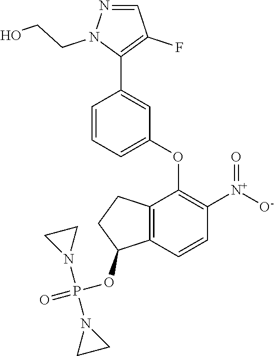 [SMILES: O=[N+]([O-])c1ccc2c(c1Oc1cccc(-c3c(F)cnn3CCO)c1)CC[C@@H]2OP(=O)(N1CC1)N1CC1] | LCMS(ESI): m/z 530.1 [M + H]⁺; ¹H NMR (400 MHz, CDCl₃) δ 7.90 (d, J = 8.2 Hz, 1H), 7.54 (d, J = 8.2 Hz, 1H), 7.49-7.38 (m, 2H), 7.16 (d, J = 7.6 Hz, 1H), 7.04-6.97 (m, 1H), 6.95 (s, 1H), 6.07-5.94 (m, 1H), 4.17-4.04 (m, 2H), 4.01- 3.87 (m, 2H), 3.11 (s, 1H), 2.98-2.86 (m, 1H), 2.75-2.63 (m, 1H), 2.62-2.49 (m, 1H), 2.34-2.12 (m, 9H). ee: 93% Retention time: 4.416 min (Method 56). |
| 372 | 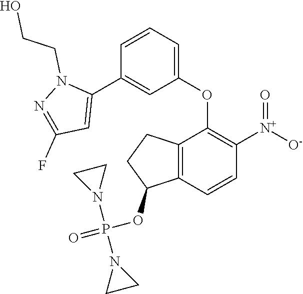 [SMILES: O=[N+]([O-])c1ccc2c(c1Oc1cccc(-c3cc(F)nn3CCO)c1)CC[C@@H]2OP(=O)(N1CC1)N1CC1] | LCMS(ESI): m/z 530.2 [M + H]⁺; ¹H NMR (400 MHz, CDCl₃) δ 7.90 (d, J = 8.2 Hz, 1H), 7.55 (d, J = 8.2 Hz, 1H), 7.40 (td, J = 7.6, 1.1 Hz, 1H), 7.17-7.09 (m, 1H), 7.04- 6.94 (m, 2H), 6.00 (td, J = 7.1, 4.6 Hz, 1H), 5.84 (d, J = 5.9 Hz, 1H), 4.03 (dd, J = 7.0, 3.0 Hz, 2H), 3.92 (dd, J = 10.2, 5.2 Hz, 2H), 2.91 (ddd, J = 15.9, 10.1, 4.4 Hz, 2H), 2.70 (ddd, J = 16.7, 8.2, 5.5 Hz, 1H), 2.56 (dd, J = 13.3, 6.4 Hz, 1H), 2.36-2.09 (m, 9H). ee: 95% Retention time: 2.331 min (Method 52). |
| 373 | 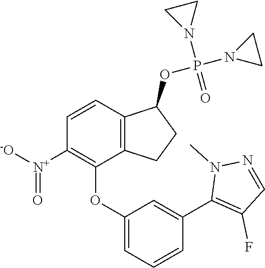 | LCMS(ESI): m/z 500.1 [M + H]⁺; ¹H NMR (400 MHz, CDCl₃) δ 7.90 (d, J = 7.8 Hz, 1H), 7.54 (d, J = 8.1 Hz, 1H), 7.47-7.40 (m, 1H), 7.39-7.34 (m, 1H), 7.15 (d, J = 7.5 Hz, 1H), 7.00-6.88 (m, 2H), 6.07-5.95 (m, 1H), 3.82 (d, J = 1.0 Hz, 3H), 3.04-2.80 (m, 1H), 2.73-2.48 (m, 2H), 2.31-2.12 (m, 9H). ee: 98% Retention time: 1.986 min (Method 52). |
| 374 | 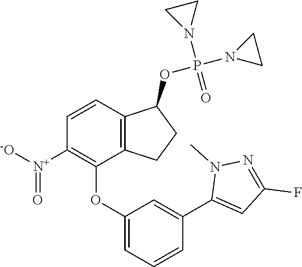 | LCMS(ESI): m/z 500.2 [M + H]⁺; ¹H NMR (400 MHz, CDCl₃) δ 7.91 (d, J = 8.2 Hz, 1H), 7.55 (d, J = 8.2 Hz, 1H), 7.45-7.32 (m, 1H), 7.11 (d, J = 7.7 Hz, 1H), 6.91 (dd, J = 7.6, 1.6 Hz, 2H), 6.02 (dd, J = 12.1, 7.0 Hz, 1H), 5.82 (d, J = 5.9 Hz, 1H), 3.72 (s, 3H), 2.92 (ddd, J = 16.0, 8.6, 4.7 Hz, 1H), 2.75-2.49 (m, 2H), 2.39-2.09 (m, 9H). ee: 96% Retention time: 2.109 min (Method 52). |
| 375 | 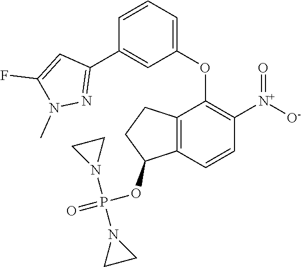 [SMILES: Cn1nc(-c2cccc(Oc3c([N+](=O)[O-])ccc4c3CC[C@@H]4OP(=O)(N3CC3)N3CC3)c2)cc1F] | LCMS(ESI): m/z 500.1 [M + H]⁺; ¹H NMR (400 MHz, CDCl₃) δ 7.89 (d, J = 8.2 Hz, 1H), 7.51 (d, J = 8.2 Hz, 1H), 7.43 (d, J = 7.9 Hz, 1H), 7.30 (dd, J = 9.3, 6.5 Hz, 2H), 6.82-6.74 (m, 1H), 6.10-5.88 (m, 2H), 3.75 (t, J = 8.0 Hz, 3H), 3.01-2.76 (m, 1H), 2.68-2.45 (m, 2H), 2.36-2.09 (m, 9H). ee: >99% Retention time: 2.636 min (Method 42). |
| 376 | 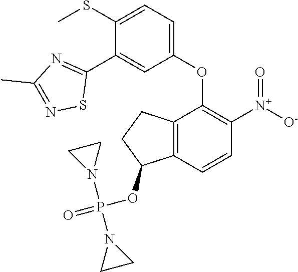 [SMILES: CSc1ccc(Oc2c([N+](=O)[O-])ccc3c2CC[C@@H]3OP(=O)(N2CC2)N2CC2)cc1-c1nc(C)ns1] | LCMS(ESI): m/z 546.1 [M + H]⁺; ¹H NMR (400 MHz, CDCl₃) δ 8.04 (d, J = 2.9 Hz, 1H), 7.95 (d, J = 8.2 Hz, 1H), 7.63-7.52 (m, 2H), 6.96-6.88 (m, 1H), 6.08-5.99 (m, 1H), 2.96-2.85 (m, 1H), 2.69 (s, 3H), 2.68-2.52 (m, 2H), 2.48 (s, 3H), 2.30-2.17 (m, 9H). |

### Illustration 46. Synthesis of (R)-4-((4-fluoro-1-methyl-1H-indol-6-yl)oxy)-5-nitro-2,3-dihydro-1H-inden-1-yl di(aziridin-1-yl)phosphinate & (S)-4-((4-fluoro-1-methyl-1H-indol-6-yl)oxy)-5-nitro-2,3-dihydro-1H-inden-1-yl di(aziridin-1-yl)phosphinate (377)

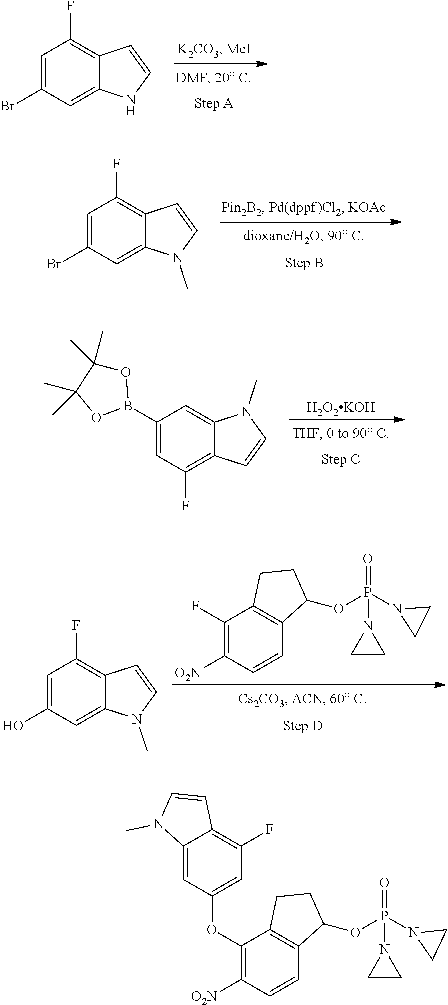

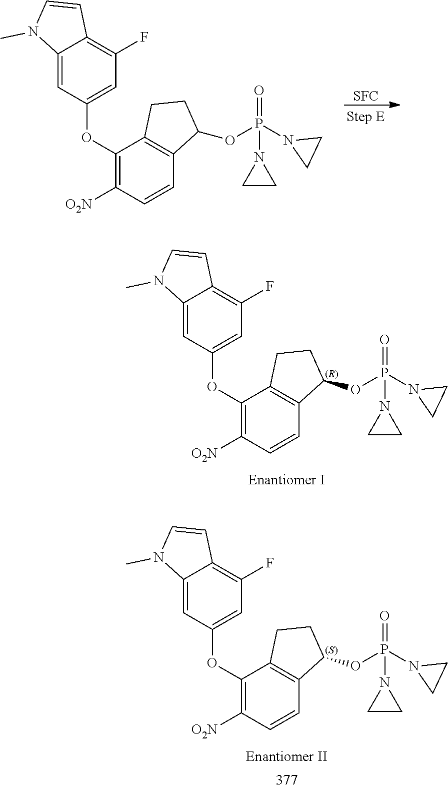

### STEP A: 6-bromo-4-fluoro-1-methyl-1H-indole

To a solution of 6-bromo-4-fluoro-1H-indole (850 mg, 3.97 mmol, 1.0 eq.) and K₂CO₃ (1.09 g, 7.94 mmol, 2.0 eq.) in DMF (10 mL) was added CH₃I (0.32 mL, 5.16 mmol, 1.3 eq.). The resulting mixture was stirred at 20° C. for 18 hrs. After completion, the reaction was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford 6-bromo-4-fluoro-1-methyl-1H-indole (790 mg, 3.46 mmol, 87%) as a yellow solid. LCMS (ESI): m/z 228 [M+H]⁺.

### STEP B: 4-fluoro-1-methyl-6-(4,4,5,5-tetramethyl-1,3,2-dioxaborolan-2-yl)-1H-indole

To a solution of 6-bromo-4-fluoro-1-methyl-1H-indole (590 mg, 2.59 mmol, 1.0 eq.) and 4,4,4′,4′,5,5,5′,5′-octamethyl-2,2′-bi (1,3,2-dioxaborolane) (919 mg, 3.62 mmol, 1.4 eq.) and KOAc (762 mg, 7.76 mmol, 3.0 eq.) in dioxane (20 mL) was added Pd(dppf)Cl₂ (95 mg, 0.13 mmol, 0.05 eq.). The resulting mixture was stirred at 90° C. for 18 hrs. After completion, the reaction mixture was concentrated under reduced pressure. The residue was purified by flash column chromatography on silica gel to afford 4-fluoro-1-methyl-6-(4,4,5,5-tetramethyl-1,3,2-dioxaborolan-2-yl)-1H-indole (40 mg, 0.18 mmol, 75%) as a yellow solid. LCMS (ESI): m/z 276 [M+H]⁺.

### STEP C: 4-fluoro-1-methyl-1H-indol-6-ol

To a solution of 4-fluoro-1-methyl-6-(4,4,5,5-tetramethyl-1,3,2-dioxaborolan-2-yl)-1H-indole (600 mg, 2.18 mmol, 1.0 eq.) in THF (10 mL) was added KOH (253 mg, 4.36 mmol, 2.0 eq.) at 0° C. H₂O₂ (494 mg×30 wt %, 4.36 mmol, 2.0 eq.) was added to the mixture dropwise at 0° C. The mixture was heated to 90° C. and stirred for 18 hrs. After completion, the reaction mixture was quenched by adding Na₂S₂O₃ (15 wt %, aq., 10 mL) and stirred for 1 hr, then separated. The aqueous layer was neutralized carefully with HCl (aq., 1M) until the pH was adjusted to pH=3-4. The resulting mixture was extracted with DCM (20 mL). The organic layers were combined and washed with brine (20 mL), dried over anhydrous Na₂SO₄, then concentrated under reduced pressure. The residue was purified by flash column chromatography on silica gel to afford 4-fluoro-1-methyl-1H-indol-6-ol (220 mg, 1.33 mmol, 61%) as a yellow solid. LCMS (ESI): m/z 166 [M+H]⁺.

### STEP D: 4-((4-fluoro-1-methyl-1H-indol-6-yl)oxy)-5-nitro-2,3-dihydro-1H-inden-1-yl di(aziridin-1-yl)phosphinate

To a solution of 4-fluoro-5-nitro-2,3-dihydro-1H-inden-1-yl di(aziridin-1-yl)phosphinate (50 mg, 0.15 mmol, 1.0 eq.) and 4-fluoro-1-methyl-1H-indol-6-ol (38 mg, 0.23 mmol, 1.5 eq.) in ACN (3 mL) was added Cs₂CO₃ (100 mg, 0.31 mmol, 2.0 eq.). The resulting mixture was stirred at 60° C. for 1 hr. After completion, the reaction mixture was concentrated under reduced pressure. The residue was purified by prep-HPLC to give 4-((4-fluoro-1-methyl-1H-indol-6-yl)oxy)-5-nitro-2,3-dihydro-1H-inden-1-yl di(aziridin-1-yl)phosphinate (50 mg, 0.11 mmol, 69%) as a yellow solid. LCMS (ESI): m/z 473.2 [M+H]⁺.

### STEP E: (R)-4-((4-fluoro-1-methyl-1H-indol-6-yl)oxy)-5-nitro-2,3-dihydro-1H-inden-1-yl di(aziridin-1-yl)phosphinate & (S)-4-((4-fluoro-1-methyl-1H-indol-6-yl)oxy)-5-nitro-2,3-dihydro-1H-inden-1-yl di(aziridin-1-yl)phosphinate

4-((4-fluoro-1-methyl-1H-indol-6-yl)oxy)-5-nitro-2,3-dihydro-1H-inden-1-yl di(aziridin-1-yl)phosphinate (70 mg, 0.14 mmol) was further separated by Chiral SFC to give:

Enantiomer I, (R)-4-((4-fluoro-1-methyl-1H-indol-6-yl)oxy)-5-nitro-2,3-dihydro-1H-inden-1-yl di(aziridin-1-yl)phosphinate (29.7 mg, 42%); Retention time: 1.217 min, >99% ee. LC-MS (ESI): m/z 473.0 [M+H]⁺; ¹H NMR (400 MHz, CDCl₃) δ 7.88 (d, J=8.2 Hz, 1H), 7.51 (d, J=8.2 Hz, 1H), 6.96 (d, J=3.2 Hz, 1H), 6.59 (d, J=1.6 Hz, 1H), 6.53-6.41 (m, 2H), 6.05-5.94 (m, 1H), 3.68 (s, 3H), 2.92-2.76 (m, 1H), 2.67-2.45 (m, 2H), 2.39-2.12 (m, 9H). ¹⁹F NMR (377 MHz, CDCl₃) δ −119.20 (s). ³¹P NMR (162 MHz, CDCl₃) δ 29.94 (s).

Enantiomer II, (R)-4-((4-fluoro-1-methyl-1H-indol-6-yl)oxy)-5-nitro-2,3-dihydro-1H-inden-1-yl di(aziridin-1-yl)phosphinate (377), (30.6 mg, 44%); Retention time: 1.434 min, 94% ee. LC-MS (ESI): m/z 473.0 [M+H]⁺; ¹H NMR (400 MHz, CDCl₃) δ 7.88 (d, J=8.2 Hz, 1H), 7.51 (d, J=8.2 Hz, 1H), 6.96 (d, J=3.2 Hz, 1H), 6.59 (d, J=1.3 Hz, 1H), 6.54-6.33 (m, 2H), 6.11-5.77 (m, 1H), 3.68 (s, 3H), 2.96-2.71 (m, 1H), 2.69-2.40 (m, 2H), 2.39-2.02 (m, 9H). ¹⁹F NMR (377 MHz, CDCl₃) δ −119.20 (s). ³¹P NMR (162 MHz, CDCl₃) δ 29.94 (s).

Analytical method: Column: ChiralCel OJ, 250×4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C.

SFC Method: Instrument: Waters Thar 80 preparative SFC, Column: ChiralCel OJ, 250×21.2 mm I.D., 5 μm, Mobile phase: A for CO₂ and B for MEOH+0.1% NH₃H₂O, Gradient: B 35%, Flow rate: 40 mL/min, Back pressure: 100 bar, Column temperature: 35° C., Wavelength: 220 nm, Cycle-time: 2.9 min, Eluted time: 2H.

TABLE 11
Characterization of compounds (see Illustration 46)

| Compound No. | Structure | LCMS; ¹H NMR (ppm); Retention time |
|---|---|---|
| 378 | 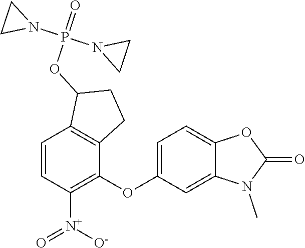 [SMILES: Cn1c(=O)oc2ccc(Oc3c([N+](=O)[O-])ccc4c3CCC4OP(=O)(N3CC3)N3CC3)cc21] | LCMS (ESI): m/z 473.1 [M + H]⁺; ¹H NMR (400 MHz, CDCl₃) δ 7.92-7.87 (m, 1H), 7.57-7.51 (m, 1H), 7.10-7.05 (m, 1H), 6.63-6.60 (m, 1H), 6.51 (dd, J = 8.7, 2.5 Hz, 1H), 6.04-5.98 (m, 1H), 3.37 (s, 3H), 2.94-2.80 (m, 1H), 2.66-2.53 (m, 2H), 2.31-2.15 (m, 9H). |
| 379 | 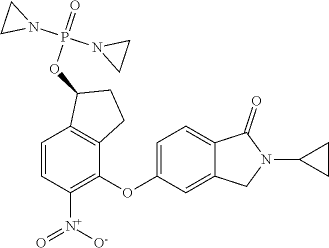 [SMILES: O=C1c2ccc(Oc3c([N+](=O)[O-])ccc4c3CC[C@@H]4OP(=O)(N3CC3)N3CC3)cc2CN1C1CC1] | LCMS (ESI): m/z 497.2 [M + H]⁺; ¹H NMR (400 MHz, CDCl₃) δ 7.93 (d, J = 8.2 Hz, 1H), 7.76 (d, J = 8.3 Hz, 1H), 7.56 (d, J = 8.2 Hz, 1H), 6.93 (dd, J = 8.4, 2.2 Hz, 1H), 6.89 (d, J = 1.7 Hz, 1H), 6.11-5.96 (m, 1H), 4.27 (s, 2H), 2.99-2.79 (m, 2H), 2.72-2.52 (m, 2H), 2.33-2.13 (m, 9H), 0.97-0.75 (m, 4H). ee: >99% Retention time: 7.211 min (Method 5). |
| 380 | 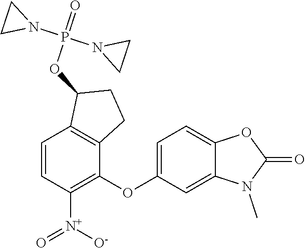 [SMILES: Cn1c(=O)oc2ccc(Oc3c([N+](=O)[O-])ccc4c3CC[C@@H]4OP(=O)(N3CC3)N3CC3)cc21] | LCMS (ESI): m/z 473.1 [M + H]⁺; ¹H NMR (400 MHz, CDCl₃) δ 7.90 (d, J = 8.2 Hz, 1H), 7.54 (d, J = 8.3 Hz, 1H), 7.08 (d, J = 8.7 Hz, 1H), 6.62 (t, J = 3.4 Hz, 1H), 6.52 (dd, J = 8.7, 2.5 Hz, 1H), 6.05-5.97 (m, 1H), 3.37 (s, 3H), 2.93-2.82 (m, 1H), 2.66-2.50 (m, 2H), 2.30-2.14 (m, 9H).. ee: >99% Retention time: 4.259 min (Method 1). |
| 381 | 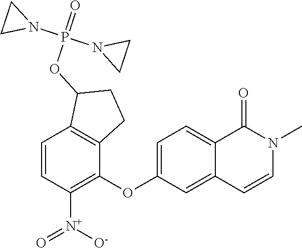 [SMILES: Cn1ccc2cc(Oc3c([N+](=O)[O-])ccc4c3CCC4OP(=O)(N3CC3)N3CC3)ccc2c1=O] | LCMS (ESI): m/z 483.1 [M + H]⁺; ¹H NMR (400 MHz, CDCl₃) δ 8.41 (d, J = 8.9 Hz, 1H), 7.95 (d, J = 8.3 Hz, 1H), 7.57 (d, J = 8.2 Hz, 1H), 7.09-7.01 (m, 2H), 6.81 (d, J = 2.4 Hz, 1H), 6.33 (d, J = 7.4 Hz, 1H), 6.08-5.97 (m, 1H), 3.58 (s, 3H), 2.95-2.79 (m, 1H), 2.72-2.48 (m, 2H), 2.39-2.13 (m, 9H). |
| 382 | 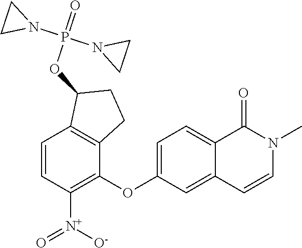 [SMILES: Cn1ccc2cc(Oc3c([N+](=O)[O-])ccc4c3CC[C@@H]4OP(=O)(N3CC3)N3CC3)ccc2c1=O] | LCMS (ESI): m/z 483.1 [M + H]⁺; ¹H NMR (400 MHz, CDCl₃) δ 8.40 (d, J = 8.9 Hz, 1H), 7.95 (d, J = 8.2 Hz, 1H), 7.57 (d, J = 8.2 Hz, 1H), 7.13-6.94 (m, 2H), 6.81 (d, J = 2.5 Hz, 1H), 6.34 (d, J = 7.4 Hz, 1H), 6.17-5.91 (m, 1H), 3.58 (s, 3H), 2.99-2.80 (m, 1H), 2.75-2.46 (m, 2H), 2.35-2.09 (m, 9H). ee: >99% Retention time: 5.941 min (Method 20). |
| 383 | 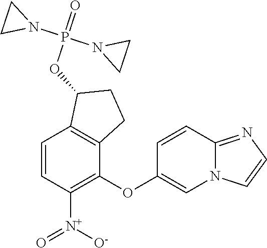 [SMILES: O=[N+]([O-])c1ccc2c(c1Oc1ccc3nccn3c1)CC[C@H]2OP(=O)(N1CC1)N1CC1] | LCMS (ESI): m/z 442.2 [M + H]⁺; ¹H NMR (400 MHz, DMSO) δ 8.27 (d, J = 2.0 Hz, 1H), 8.07 (d, J = 8.2 Hz, 1H), 7.85 (s, 1H), 7.63-7.55 (m, 3H), 7.20-7.15 (m, 1H), 5.98- 5.89 (m, 1H), 2.89-2.77 (m, 1H), 2.70-2.52 (m, 2H), 2.18-2.10 (m, 9H). ee: >99% Retention time: 4.360 min (Method 5). |
| 384 | 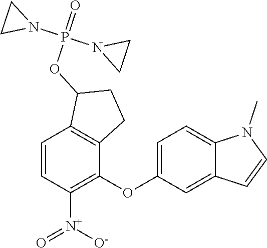 [SMILES: Cn1ccc2cc(Oc3c([N+](=O)[O-])ccc4c3CCC4OP(=O)(N3CC3)N3CC3)ccc21] | LCMS (ESI): m/z 455.1 [M + H]⁺; ¹H NMR (400 MHz, CDCl₃) δ 7.84 (d, J = 8.2 Hz, 1H), 7.44 (d, J = 8.2 Hz, 1H), 7.25-7.22 (m, 1H), 7.08-7.00 (m, 2H), 6.97-6.89 (m, 1H), 6.38-6.33 (m, 1H), 6.01-5.93 (m, 1H), 3.78 (s, 3H), 2.81-2.72 (m, 1H), 2.56-2.42 (m, 2H), 2.27-2.11 (m, 9H). |
| 385 | 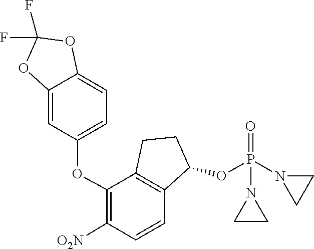 [SMILES: O=[N+]([O-])c1ccc2c(c1Oc1ccc3c(c1)OC(F)(F)O3)CC[C@@H]2OP(=O)(N1CC1)N1CC1] | LCMS (ESI): m/z 482.10 [M + H]⁺; ¹H NMR (400 MHz, DMSO-d₆) δ 8.05 (d, J = 8.2 Hz, 1H), 7.55 (d, J = 8.2 Hz, 1H), 7.37 (d, J = 8.8 Hz, 1H), 7.21 (d, J = 2.7 Hz, 1H), 6.72 (dd, J = 8.9, 2.6 Hz, 1H), 5.97-5.90 (m, 1H), 2.79-2.67 (m, 1H), 2.62-2.53 (m, 2H), 2.18-2.04 (m, 9H). ee: >99% Retention time: 1.957 min; Column: Lux 3 u Cellulose-4, 0.46 × 5 cm, 3.0 um; Mobile phase: Hex (0.1% DEA):IPA = 50:50, Flow rate: 1.67 mL/min; Temperature: ambient. |
| 386 | 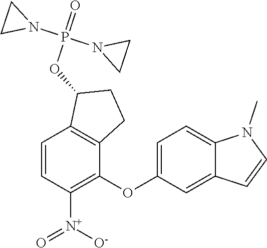 [SMILES: Cn1ccc2cc(Oc3c([N+](=O)[O-])ccc4c3CC[C@H]4OP(=O)(N3CC3)N3CC3)ccc21] | LCMS (ESI): m/z 455.1 [M + H]⁺; ¹H NMR (400 MHz, CDCl₃) δ 7.84 (d, J = 8.2 Hz, 1H), 7.44 (d, J = 8.2 Hz, 1H), 7.25-7.21 (m, 1H), 7.07-7.01 (m, 2H), 6.96-6.90 (m, 1H), 6.38-6.35 (m, 1H), 6.01-5.92 (m, 1H), 3.78 (s, 3H), 2.82-2.71 (m, 1H), 2.55-2.43 (m, 2H), 2.27-2.12 (m, 9H). ee: >99% Retention time: 5.837 min (Method 1). |
| 387 | 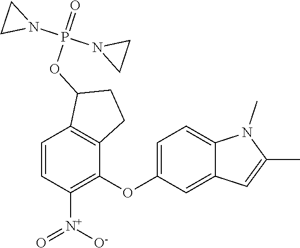 [SMILES: Cc1cc2cc(Oc3c([N+](=O)[O-])ccc4c3CCC4OP(=O)(N3CC3)N3CC3)ccc2n1C] | LCMS (ESI): m/z 469.1 [M + H]⁺; ¹H NMR (400 MHz, CDCl₃) δ 7.82 (d, J = 8.2 Hz, 1H), 7.46-7.36 (m, 1H), 7.15 (d, J = 8.8 Hz, 1H), 7.00-6.87 (m, 1H), 6.86-6.79 (m, 1H), 6.14 (s, 1H), 6.02-5.85 (m, 1H), 3.65 (s, 3H), 2.89-2.61 (m, 1H), 2.53- 2.42 (m, 2H), 2.40 (s, 3H), 2.27-2.09 (m, 9H). |
| 388 | 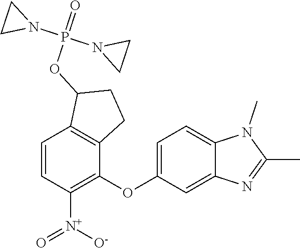 [SMILES: Cc1nc2cc(Oc3c([N+](=O)[O-])ccc4c3CCC4OP(=O)(N3CC3)N3CC3)ccc2n1C] | LCMS (ESI): m/z 470.1 [M + H]⁺; ¹H NMR (400 MHz, CDCl₃) δ 7.86 (d, J = 6.2 Hz, 1H), 7.47 (d, J = 8.2 Hz, 1H), 7.23-7.18 (m, 1H), 7.10-7.06 (m, 1H), 6.99-6.92 (m, 1H), 6.01-5.92 (m, 1H), 3.72 (s, 3H), 2.86-2.70 (m, 1H), 2.59 (s, 3H), 2.56- 2.43 (m, 2H), 2.27-2.14 (m, 9H). |
| 389 | 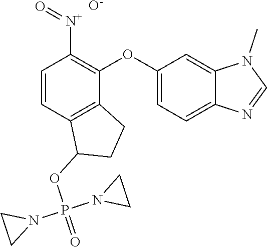 | LCMS (ESI): m/z 456.1 [M + H]⁺; ¹H NMR (400 MHz, CDCl₃) δ 7.96-7.87 (m, 2H), 7.72 (d, J = 9.5 Hz, 1H), 7.52 (d, J = 8.3 Hz, 1H), 6.94-6.87 (m, 2H), 6.04-5.95 (m, 1H), 3.79 (s, 3H), 2.90-2.78 (m, 1H), 2.64-2.47 (m, 2H), 2.29-2.11 (m, 9H). |
| 390 | 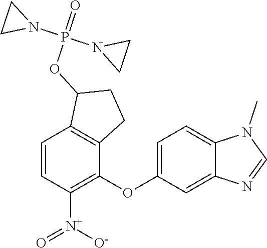 [SMILES: Cn1cnc2cc(Oc3c([N+](=O)[O-])ccc4c3CCC4OP(=O)(N3CC3)N3CC3)ccc21] | LCMS (ESI): m/z 456.1 [M + H]⁺; ¹H NMR (400 MHz, CDCl₃) δ 7.88 (d, J = 8.2 Hz, 1H), 7.83 (s, 1H), 7.49 (d, J = 8.2 Hz, 1H), 7.32 (d, J = 8.8 Hz, 1H), 7.16-7.13 (dd, J = 9.7, 2.3 Hz, 1H), 7.09-7.05 (m, 1H), 6.01-5.96 (m, 1H), 3.84 (s, 3H), 2.88-2.75 (m, 1H), 2.61-2.46 (m, 2H), 2.29-2.14 (m, 9H). |
| 391 | 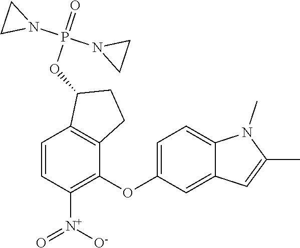 [SMILES: Cc1cc2cc(Oc3c([N+](=O)[O-])ccc4c3CC[C@H]4OP(=O)(N3CC3)N3CC3)ccc2n1C] | LCMS (ESI): m/z 469.1 [M + H]⁺; ¹H NMR (400 MHz, CDCl₃) δ 7.82 (d, J = 8.2 Hz, 1H), 7.42 (d, J = 8.2 Hz, 1H), 7.15 (d, J = 8.8 Hz, 1H), 6.94 (d, J = 2.3 Hz, 1H), 6.88- 6.73 (m, 1H), 6.14 (s, 1H), 5.99-5.90 (m, 1H), 3.65 (s, 3H), 2.79-2.67 (m, 1H), 2.51-2.42 (m, 2H), 2.40 (s, 3H), 2.29-2.11 (m, 9H). ee: 98% Retention time: 4.933 min (Method 9). |
| 392 | 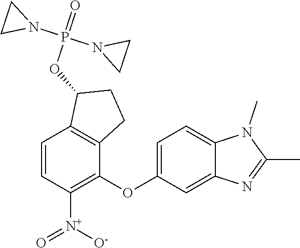 [SMILES: Cc1nc2cc(Oc3c([N+](=O)[O-])ccc4c3CC[C@H]4OP(=O)(N3CC3)N3CC3)ccc2n1C] | LCMS (ESI): m/z 470.1 [M + H]⁺; ¹H NMR (400 MHz, CDCl₃) δ 7.87 (d, J = 8.2 Hz, 1H), 7.48 (d, J = 8.3 Hz, 1H), 7.21 (d, J = 8.7 Hz, 1H), 7.09 (d, J = 2.1 Hz, 1H), 7.00- 6.94 (m, 1H), 6.01-5.92 (m, 1H), 3.73 (s, 3H), 2.86-2.75 (m, 1H), 2.60 (s, 3H), 2.57-2.42 (m, 2H), 2.29-2.15 (m, 9H). ee: 94% Retention time: 7.538 min (Method 27). |
| 393 | 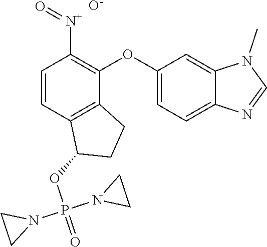 | LCMS (ESI): m/z 456.1 [M + H]⁺; ¹H NMR (400 MHz, CDCl₃) δ 7.89 (d, J = 8.2 Hz, 1H), 7.83 (s, 1H), 7.70 (d, J = 9.5 Hz, 1H), 7.51 (d, J = 8.2 Hz, 1H), 6.91-6.84 (m, 2H), 6.03-5.97 (m, 1H), 3.77 (s, 3H), 2.87-2.76 (m, 1H), 2.61-2.48 (m, 2H), 2.29-2.16 (m, 9H). ee: >99% Retention time: 1.839 min (Method 28). |
| 394 | 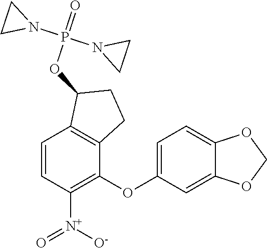 [SMILES: O=[N+]([O-])c1ccc2c(c1Oc1ccc3c(c1)OCO3)CC[C@@H]2OP(=O)(N1CC1)N1CC1] | LCMS (ESI): m/z 446.0 [M + H]⁺; ¹H NMR (400 MHz, CDCl₃) δ 7.84 (d, J = 8.2 Hz, 1H), 7.47 (d, J = 8.2 Hz, 1H), 6.68 (d, J = 8.5 Hz, 1H), 6.50 (d, J = 2.5 Hz, 1H), 6.30- 6.25 (m, 1H), 6.03-5.92 (m, 3H), 2.97-2.80 (m, 1H), 2.67-2.49 (m, 2H), 2.29-2.16 (m, 9H). ee: 98% Retention time: 3.934 min (Method 1). |
| 395 | 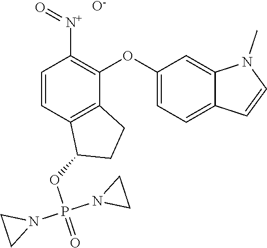 | LCMS (ESI): m/z 455.1 [M + H]⁺; ¹H NMR (400 MHz, CDCl₃) δ 7 7.98 (d, J = 8.2 Hz, 1H), 7.53-7.44 (m, 2H), 7.26 (t, J = 3.7 Hz, 1H), 6.97 (t, J = 5.2 Hz, 1H), 6.65 (dd, J = 8.6, 2.3 Hz, 1H), 6.41-6.35 (m, 1H), 5.95-5.84 (m, 1H), 3.68 (s, 3H), 2.67-2.58 (m, 1H), 2.48-2.37 (m, 2H), 2.17-1.95 (m, 9H). ee: >99% Retention time: 5.196 min (Method 1). |
| 396 | 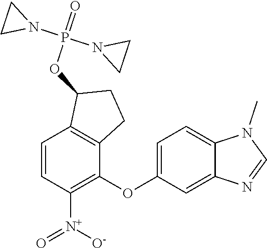 [SMILES: Cn1cnc2cc(Oc3c([N+](=O)[O-])ccc4c3CC[C@@H]4OP(=O)(N3CC3)N3CC3)ccc21] | LCMS (ESI): m/z 456.2 [M + H]⁺; ¹H NMR (400 MHz, CDCl₃) δ 7.92-7.81 (m, 2H), 7.49 (d, J = 8.1 Hz, 1H), 7.33 (d, J = 8.6 Hz, 1H), 7.16 (s, 1H), 7.07 (d, J = 8.7 Hz, 1H), 6.07-5.89 (m, 1H), 3.84 (s, 3H), 2.85-2.80 (m, 1H), 2.59-2.50 (m, 2H), 2.25-2.15 (m, 9H). ee: 96% Retention time: 4.703 min (Method 33). |
| 397 | 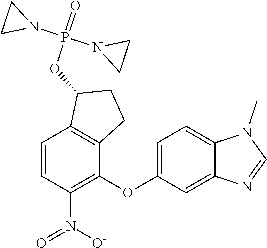 [SMILES: Cn1cnc2cc(Oc3c([N+](=O)[O-])ccc4c3CC[C@H]4OP(=O)(N3CC3)N3CC3)ccc21] | LCMS (ESI): m/z 456.2 [M + H]⁺; ¹H NMR (400 MHz, CDCl₃) δ 7.92-7.81 (m, 2H), 7.49 (d, J = 8.4 Hz, 1H), 7.32 (d, J = 8.7 Hz, 1H), 7.16 (s, 1H), 7.07 (d, J = 8.4 Hz, 1H), 6.00-5.96 (m, 1H), 3.84 (s, 3H), 2.89-2.76 (m, 1H), 2.59-2.50 (m, 2H), 2.25-2.20 (m, 9H). ee: 96% Retention time: 5.688 min (Method 33). |
| 398 | 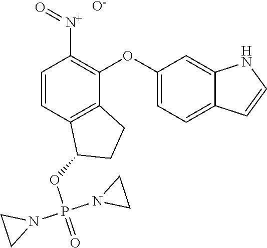 | LCMS (ESI): m/z 441.0 [M + H]⁺; ¹H NMR (400 MHz, CDCl₃) δ 8.10 (s, 1H), 7.85 (d, J = 8.2 Hz, 1H), 7.54 (d, J = 8.6 Hz, 1H), 7.47 (d, J = 8.2 Hz, 1H), 7.20-7.12 (m, 1H), 6.87 (d, J = 2.0 Hz, 1H), 6.82-6.75 (m, 1H), 6.52 (s, 1H), 6.03-5.93 (m, 1H), 2.86-2.74 (m, 1H), 2.62-2.42 (m, 2H), 2.31-2.11 (m, 9H). ee: >99% Retention time: 2.341 min (Method 1). |
| 399 | 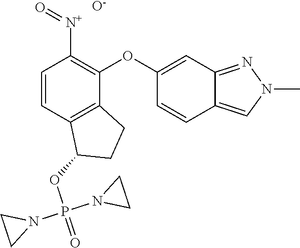 | LCMS (ESI): m/z 456.2 [M + H]⁺; ¹H NMR (400 MHz, CDCl₃) δ 7.91 (d, J = 8.2 Hz, 1H), 7.85 (s, 1H), 7.62 (d, J = 9.0 Hz, 1H), 7.52 (d, J = 8.2 Hz, 1H), 6.96-6.91 (m, 1H), 6.79 (d, J = 2.0 Hz, 1H), 6.02-5.95 (m, 1H), 4.15 (s, 3H), 2.94-2.85 (m, 1H), 2.71-2.49 (m, 2H), 2.29-2.17 (m, 9H). ee: >99% Retention time: 1.631 min (Method 42). |
| 400 | 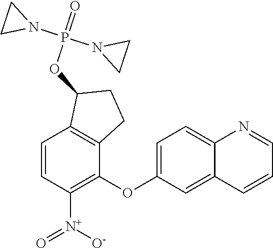 [SMILES: O=[N+]([O-])c1ccc2c(c1Oc1ccc3ncccc3c1)CC[C@@H]2OP(=O)(N1CC1)N1CC1] | LCMS (ESI): m/z 453.2 [M + H]⁺; ¹H NMR (400 MHz, CDCl₃) δ 8.88-8.80 (m, 1H), 8.11 (d, J = 9.2 Hz, 1H), 7.97 (t, J = 8.6 Hz, 2H), 7.58 (d, J = 8.2 Hz, 1H), 7.48 (dd, J = 9.2, 2.8 Hz, 1H), 7.38 (dd, J = 8.3, 4.2 Hz, 1H), 7.00 (d, J = 2.7 Hz, 1H), 6.09-5.99 (m, 1H), 2.95-2.84 (m, 1H), 2.69-2.51 (m, 2H), 2.29- 2.19 (m, 9H). ee: >99% Retention time: 5.595 min (Method 39). |
| 401 | 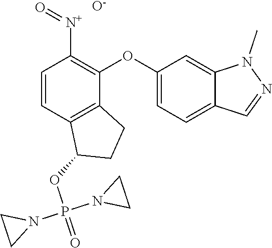 | LCMS (ESI): m/z 456.3 [M + H]⁺; ¹H NMR (400 MHz, CDCl₃) δ 8.01-7.84 (m, 2H), 7.64 (d, J = 8.8 Hz, 1H), 7.55 (d, J = 8.2 Hz, 1H), 6.86-6.77 (m, 1H), 6.73 (s, 1H), 6.09- 5.97 (m, 1H), 3.96 (s, 3H), 2.97-2.73 (m, 1H), 2.69-2.49 (m, 2H), 2.32-2.12 (m, 9H). ee: >99% Retention time: 6.407 min (Method 1). |
| 402 | 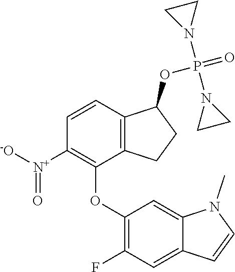 | LCMS (ESI): m/z 473.2 [M + H]⁺; ¹H NMR (400 MHz, CDCl₃) δ 7.87 (d, J = 8.2 Hz, 1H), 7.47 (d, J = 8.2 Hz, 1H), 7.35 (d, J = 11.1 Hz, 1H), 7.03 (d, J = 3.1 Hz, 1H), 6.74 (d, J = 6.7 Hz, 1H), 6.42 (d, J = 2.8 Hz, 1H), 6.00-5.93 (m, 1H), 3.66 (s, 3H), 2.84-2.76 (m, 1H), 2.59-2.45 (m, 2H), 2.26-2.14 (m, 9H). ee: 96% Retention time: 2.558 min (Method 3). |

### Illustration 47. Synthesis of (R)-4-(4-fluoro-3-(1H-pyrazol-1-yl)phenoxy)-5-nitro-2,3-dihydro-1H-inden-1-yl di(aziridin-1-yl)phosphinate & (S)-4-(4-fluoro-3-(1H-pyrazol-1-yl)phenoxy)-5-nitro-2,3-dihydro-1H-inden-1-yl di(aziridin-1-yl)phosphinate (403)

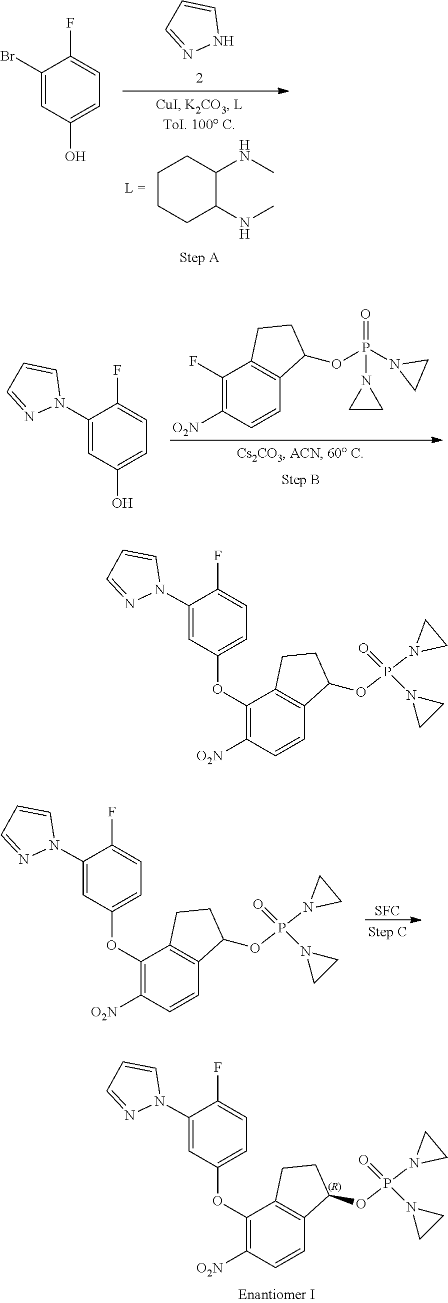

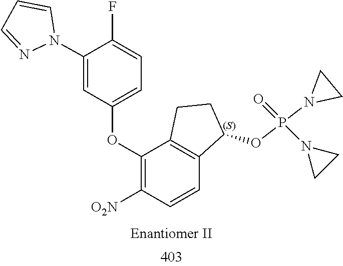

### STEP A: 4-fluoro-3-(1H-pyrazol-1-yl)phenol

To a solution of 3-bromo-4-fluorophenol (500 mg, 2.62 mmol, 1.0 eq.), 1H-pyrazole (214 mg, 3.14 mmol, 1.2 eq.) in tolune (20 mL) were added CuI (498 mg, 2.62 mmol, 1.0 eq.), N₁,N₂-dimethylcyclohexane-1,2-diamine (744 mg, 5.24 mmol, 2.0 eq.) and K₂CO₃ (1.08 g, 7.86 mmol, 3.0 eq.). The resulting mixture was stirred at 110° C. overnight under N₂. After completion, the reaction mixture was diluted with H₂O (20 mL) and extracted with EtOAc (20 mL×3). The organic layers were combined and washed with brine (10 mL), dried over anhydrous Na₂SO₄, and concentrated under reduced pressure. The residue was purified by flash column chromatography on silica gel to afford 4-fluoro-3-(1H-pyrazol-1-yl)phenol (190 mg, 1.07 mmol, 41%) as a brown oil. LCMS (ESI): m/z 179 [M+H]⁺.

### STEP B: 4-(4-fluoro-3-(1H-pyrazol-1-yl)phenoxy)-5-nitro-2,3-dihydro-1H-inden-1-yl di(aziridin-1-yl)phosphinate

To a solution of 4-fluoro-5-nitro-2,3-dihydro-1H-inden-1-yl di(aziridin-1-yl)phosphinate (50 mg, 0.15 mmol, 1.0 eq.) and 4-fluoro-3-(1H-pyrazol-1-yl)phenol (41 mg, 0.23 mmol, 1.5 eq.) in ACN (5 mL) was added Cs₂CO₃ (100 mg, 0.31 mmol, 2.0 eq.). The resulting mixture was stirred at 60° C. for 1 hr. After completion, the reaction mixture was concentrated under reduced pressure. The residue was purified by prep-HPLC to give 4-(4-fluoro-3-(1H-pyrazol-1-yl)phenoxy)-5-nitro-2,3-dihydro-1H-inden-1-yl di(aziridin-1-yl)phosphinate (50 mg, 0.10 mmol, 67%) as a yellow solid. LCMS (ESI): m/z 486 [M+H]⁺.

### STEP C: (R)-4-(4-fluoro-3-(1H-pyrazol-1-yl)phenoxy)-5-nitro-2,3-dihydro-1H-inden-1-yl di(aziridin-1-yl)phosphinate & (S)-4-(4-fluoro-3-(1H-pyrazol-1-yl)phenoxy)-5-nitro-2,3-dihydro-1H-inden-1-yl di(aziridin-1-yl)phosphinate (Peak 2 was confirmed as (S) enantiomer)

4-(4-fluoro-3-(1H-pyrazol-1-yl)phenoxy)-5-nitro-2,3-dihydro-1H-inden-1-yl di(aziridin-1-yl)phosphinate (50 mg, 0.10 mmol) was further separated by Chiral SFC to give:

Enantiomer I, (R)-4-(4-fluoro-3-(1H-pyrazol-1-yl)phenoxy)-5-nitro-2,3-dihydro-1H-inden-1-yl di(aziridin-1-yl)phosphinate (20 mg, 40%); Retention time: 2.500 min, >99% ee. LC-MS (ESI): m/z 486.1 [M+H]⁺; ¹H NMR (400 MHz, CDCl₃) δ 8.04 (t, J=2.7 Hz, 1H), 7.90 (d, J=8.2 Hz, 1H), 7.68 (d, J=1.6 Hz, 1H), 7.53 (d, J=8.2 Hz, 1H), 7.50-7.46 (m, 1H), 7.20-7.12 (m, 1H), 6.78-6.71 (m, 1H), 6.48-6.44 (m, 1H), 6.03-5.97 (m, 1H), 2.96-2.84 (m, 1H), 2.72-2.51 (m, 2H), 2.28-2.17 (m, 9H). ¹⁹F NMR (376 MHz, CDCl₃) δ −131.81 (s). ³¹P NMR (162 MHz, CDCl₃) δ 29.89 (s).

Enantiomer II, (S)-4-(4-fluoro-3-(1H-pyrazol-1-yl)phenoxy)-5-nitro-2,3-dihydro-1H-inden-1-yl di(aziridin-1-yl)phosphinate (403, 20 mg, 40%); Retention time: 2.882 min, >99% ee. LC-MS (ESI): m/z 486.1 [M+H]⁺; ¹H NMR (400 MHz, CDCl₃) δ 8.03 (t, J=2.7 Hz, 1H), 7.89 (d, J=8.2 Hz, 1H), 7.68 (d, J=1.6 Hz, 1H), 7.53 (d, J=8.2 Hz, 1H), 7.50-7.45 (m, 1H), 7.20-7.13 (m, 1H), 6.79-6.70 (m, 1H), 6.47-6.44 (m, 1H), 6.04-5.96 (m, 1H), 2.96-2.85 (m, 1H), 2.70-2.52 (m, 2H), 2.28-2.17 (m, 9H). ¹⁹F NMR (376 MHz, CDCl₃) δ −131.81 (s). ³¹P NMR (162 MHz, CDCl₃) δ 29.89 (s).

Analytical method: Column: ChiralPak AD, 100;Á4.6 mm I.D., 3 μm, Mobile phase: A for CO₂ and B for methanol (0.05% DEA), Gradient: 8 min @B 20%, Flow rate: 2.5 mL/min, Back pressure: 100 bar, Column temperature: 35° C.

SFC Method: Instrument: MG II preparative SFC (SFC-13), Column: ChiralPak AD, 250×30 mm I.D., 10 μm, Mobile phase: A for CO₂ and B for Ethanol (0.1% NH₃H₂O), Gradient: B 25%, Flow rate: 70 mL/min, Back pressure: 100 bar, Column temperature: 38° C., Wavelength: 220 nm, Cycle time: ˜5 min.

TABLE 12
Characterization of compounds (see Illustration 47)

| Compound No. | Structure | LCMS; ¹H NMR (ppm); Retention time |
|---|---|---|
| 404 | 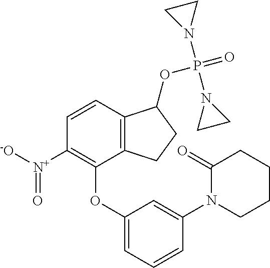 | LCMS (ESI): m/z 499.2 [M + H]⁺; ¹H NMR (400 MHz, CDCl₃) δ 7.92-7.83 (m, 1H), 7.50 (d, J = 8.2 Hz, 1H), 7.31 (t, J = 8.1 Hz, 1H), 7.04-6.94 (m, 1H), 6.81 (t, J = 2.2 Hz, 1H), 6.82-6.81 (m, 1H), 6.77-6.74 (m, 1H), 3.68-3.58 (m, 2H), 2.94-2.86 (m, 1H), 2.72-2.46 (m, 4H), 2.31-2.14 (m, 9H), 1.95-1.92 (m, 4H). |
| 405 | 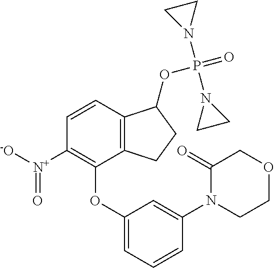 | LCMS (ESI): m/z 501.2 [M + H]⁺; ¹H NMR (400 MHz, CDCl₃) δ 7.88 (d, J = 8.2 Hz, 1H), 7.52 (d, J = 8.2 Hz, 1H), 7.34 (t, J = 8.2 Hz, 1H), 7.10-7.02 (m, 1H), 6.92 (t, J = 2.2 Hz, 1H), 6.79-6.77 (m, 1H), 6.03-6.59 (m, 1H), 4.30 (s, 2H), 4.02 (t, J = 5.1 Hz, 2H), 3.83-3.67 (m, 2H), 2.96-2.87 (m, 1H), 2.75-2.50 (m, 2H), 2.34-2.12 (m, 9H). |
| 406 | 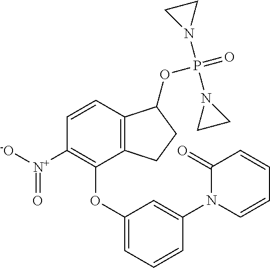 | LCMS (ESI): m/z 495.2 [M + H]⁺; ¹H NMR (400 MHz, CDCl₃) δ 7.82 (d, J = 8.2 Hz, 1H), 7.46 (d, J = 8.2 Hz, 1H), 7.40-7.22 (m, 3H), 7.06-7.00 (m, 1H), 6.91-6.83 (m, 2H), 6.57- 6.51 (m, 1H), 6.19-6.13 (m, 1H), 5.98-5.90 (m, 1H), 2.94-2.83 (m, 1H), 2.70-2.60 (m, 1H), 2.58-2.47 (m, 1H), 2.23-2.08 (m, 9H). |
| 407 | 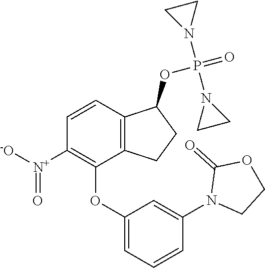 | LCMS (ESI): m/z 487.1 [M + H]⁺; ¹H NMR (400 MHz, CDCl₃) δ 7.89 (d, J = 8.2 Hz, 1H), 7.52 (d, J = 8.2 Hz, 1H), 7.33-7.23 (m, 2H), 7.21-7.15 (m, 1H), 6.62-6.55 (m, 1H), 6.04- 5.94 (m, 1H), 4.47 (t, J = 8.0 Hz, 2H), 4.09-3.97 (m, 2H), 2.97-2.83 (m, 1H), 2.71-2.49 (m, 2H), 2.29-2.15 (m, 9H). ee: >99% Retention time: 5.822 min (Method 1). |
| 408 | 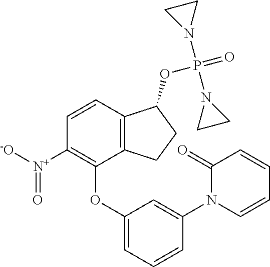 | LCMS (ESI): m/z 495.1 [M + H]⁺; ¹H NMR (400 MHz, CDCl₃) δ 7.89 (d, J = 8.2 Hz, 1H), 7.53 (d, J = 8.2 Hz, 1H), 7.47-7.24 (m, 3H), 7.13-7.05 (m, 1H), 6.98-6.88 (m, 2H), 6.61 (d, J = 9.2 Hz, 1H), 6.28-6.19 (m, 1H), 6.05-5.96 (m, 1H), 3.02-2.88 (m, 1H), 2.78-2.65 (m, 1H), 2.65-2.53 (m, 1H), 2.31-2.14 (m, 9H). ee: >99% Retention time: 4.841 min (Method 9). |
| 409 | 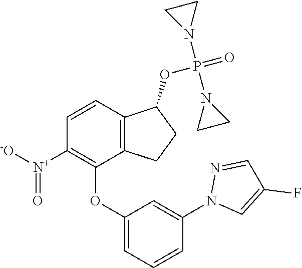 | LCMS (ESI): m/z 486.1 [M + H]⁺; ¹H NMR (400 MHz, CDCl₃) δ 7.92 (d, J = 8.2 Hz, 1H), 7.78 (d, J = 4.8 Hz, 1H), 7.58-7.52 (m, 2H), 7.36 (t, J = 8.1 Hz, 1H), 7.30-7.29 (m, 1H), 7.25 (s, 1H), 6.76-6.74 (m, 1H), 6.02-6.01 (m, 1H), 2.9-2.88 (m, 1H), 2.70-2.53 (m, 2H), 2.30-2.17 (m, 9H). ee: >99% Retention time: 3.272 min (Method 9). |
| 410 | 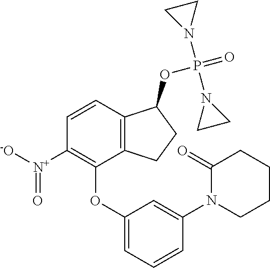 | LCMS (ESI): m/z 499.2 [M + H]⁺; ¹H NMR (400 MHz, CDCl₃) δ 7.87 (d, J = 8.2 Hz, 1H), 7.61-7.44 (m, 1H), 7.31 (t, J = 7.5 Hz, 1H), 6.98 (d, J = 7.8 Hz, 1H), 6.84-6.79 (m, 1H), 6.77-6.74 (m, 1H), 6.02-5.97 (m, 1H), 3.72-3.51 (m, 2H), 2.94- 2.86 (m, 1H), 2.77-2.44 (m, 4H), 2.35-2.08 (m, 9H), 2.04-1.85 (m, 4H). ee: >99% Retention time: 4.414 min (Method 1). |
| 411 | 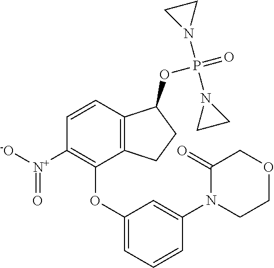 | LCMS (ESI): m/z 501.1 [M + H]⁺; ¹H NMR (400 MHz, CDCl₃) δ 7.88 (d, J = 8.2 Hz, 1H), 7.52 (d, J = 8.1 Hz, 1H), 7.34 (t, J = 8.2 Hz, 1H), 7.08-7.03 (m, 1H), 6.95-6.89 (m, 1H), 6.81-6.75 (m, 1H), 6.06-5.97 (m, 1H), 4.30 (s, 2H), 4.01 (t, J = 5.0 Hz, 2H), 3.80-3.71 (m, 2H), 2.96-2.87 (m, 1H), 2.72-2.54 (m, 2H), 2.31-2.16 (m, 9H). ee: >99% Retention time: 5.180 min (Method 1). |
| 412 | 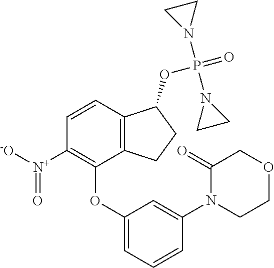 | LCMS (ESI): m/z 501.1 [M + H]⁺; ¹H NMR (400 MHz, CDCl₃) δ 7.88 (d, J = 8.2 Hz, 1H), 7.52 (d, J = 8.1 Hz, 1H), 7.34 (t, J = 8.2 Hz, 1H), 7.09-7.03 (m, 1H), 6.95-6.90 (m, 1H), 6.81-6.74 (m, 1H), 6.05-5.96 (m, 1H), 4.30 (s, 2H), 4.01 (t, J = 5.0 Hz, 2H), 3.79-3.71 (m, 2H), 2.96-2.85 (m, 1H), 2.71-2.53 (m, 2H), 2.33-2.15 (m, 9H). ee: 97% Retention time: 5.933 min (Method 1). |
| 413 | 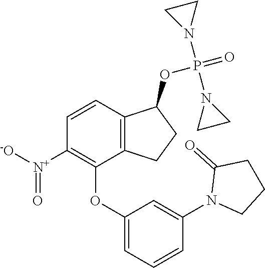 | LCMS (ESI): m/z 485.2 [M + H]⁺; ¹H NMR (400 MHz, CDCl₃) δ 7.88 (d, J = 8.2 Hz, 1H), 7.51 (t, J = 5.7 Hz, 1H), 7.38-7.36 (m, 1H), 7.29-7.27 (m, 2H), 6.64-6.57 (m, 1H), 6.03- 5.98 (m, 1H), 3.83-3.81 (m, 2H), 2.88-2.86 (m, 1H), 2.70-2.52 (m, 4H), 2.29-2.11 (m, 11H). ee: >99% Retention time: 6.743 min (Method 1). |
| 414 | 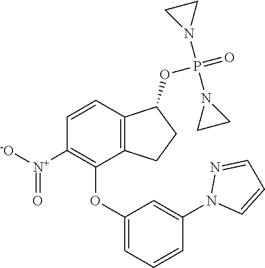 | LCMS (ESI): m/z 468.1 [M + H]⁺; ¹H NMR (400 MHz, CDCl₃) δ 7.96-7.87 (m, 2H), 7.73-7.67 (m, 1H), 7.54 (d, J = 8.2 Hz, 1H), 7.42-7.35 (m, 2H), 7.34-7.31 (m, 1H), 6.78- 6.71 (m, 1H), 6.49-6.43 (m, 1H), 6.05-5.96 (m, 1H), 2.97-2.86 (m, 1H), 2.69-2.54 (m, 2H), 2.28-2.18 (m, 9H). ee: >99% Retention time: 2.466 min (Method 7). |
| 415 | 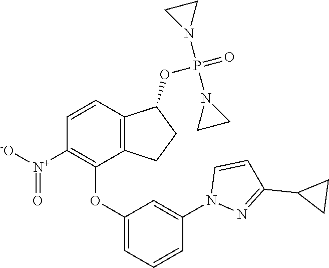 | LCMS (ESI): m/z 508.2 [M + H]⁺; ¹H NMR (400 MHz, CDCl₃) δ 7.91 (d, J = 8.2 Hz, 1H), 7.75 (d, J = 2.5 Hz, 1H), 7.53 (d, J = 7.9 Hz, 1H), 7.35-7.29 (m, 3H), 6.68-6.62 (m, 1H), 6.09 (d, J = 2.4 Hz, 1H), 6.03-5.98 (m, 1H), 2.94-2.83 (m, 1H), 2.69- 2.52 (m, 2H), 2.29-2.18 (m, 9H), 2.06-1.97 (m, 1H), 0.99-0.93 (m, 2H), 0.81-0.76 (m, 2H). ee: >99% Retention time: 4.157 min (Method 15). |
| 416 | 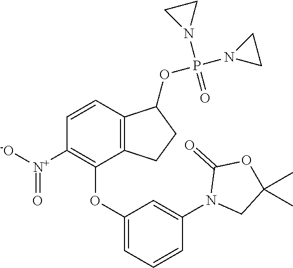 | LCMS (ESI): m/z 515.1 [M + H]⁺; ¹H NMR (400 MHz, CDCl₃) δ 7.89 (d, J = 8.2 Hz, 1H), 7.54-7.48 (m, 1H), 7.32-7.27 (m, 1H), 7.26-7.22 (m, 1H), 7.20-7.13 (m, 1H), 6.61-6.51 (m, 1H), 6.06-5.94 (m, 1H), 3.77-3.70 (m, 2H), 2.95-2.85 (m, 1H), 2.70- 2.52 (m, 1H), 2.31-2.14 (m, 9H), 1.58 (s, 6H). |
| 417 | 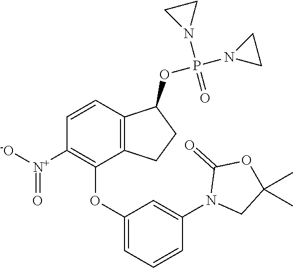 | LCMS (ESI): m/z 515.2 [M + H]⁺; ¹H NMR (400 MHz, CDCl₃) δ 7.89 (d, J = 8.2 Hz, 1H), 7.52 (d, J = 8.2 Hz, 1H), 7.29-7.27 (m, 1H), 7.24 (s, 1H), 7.19-7.15 (m, 1H), 6.58-6.53 (m, 1H), 6.05-5.96 (m, 1H), 3.74 (d, 2H), 2.96-2.84 (m, 1H), 2.70-2.52 (m, 2H), 2.30-2.13 (m, 9H), 1.55 (s, 6H). ee: >99% Retention time: 3.783 min (Method 1). |
| 418 | 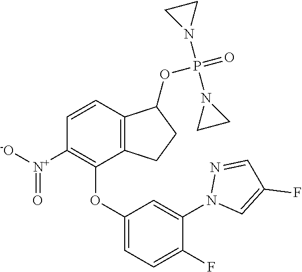 | LCMS (ESI): m/z 504.1 [M + H]⁺; ¹H NMR (400 MHz, CDCl₃) δ 7.96-7.93 (m, 1H), 7.91 (d, J = 8.2 Hz, 1H), 7.57-7.52 (m, 2H), 7.47-7.42 (m, 1H), 7.20-7.12 (m, 1H), 6.79-6.72 (m, 1H), 6.05-5.97 (m, 1H), 2.98-2.87 (m, 1H), 2.72-2.53 (m, 2H), 2.30- 2.18 (m, 9H). |
| 419 | 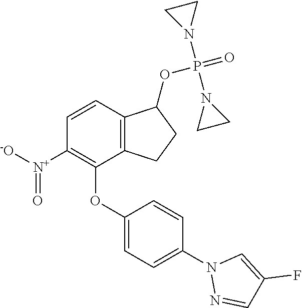 | LCMS (ESI): m/z 486.1 [M + H]⁺; ¹H NMR (400 MHz, CDCl₃) δ 7.90 (d, J = 8.2 Hz, 1H), 7.73 (d, J = 4.8 Hz, 1H), 7.68-7.42 (m, 4H), 6.95 (d, J = 9.0 Hz, 2H), 6.04-5.99 (m, 1H), 3.00-2.73 (m, 1H), 2.81-2.44 (m, 2H), 2.42-2.07 (m, 9H). |
| 420 | 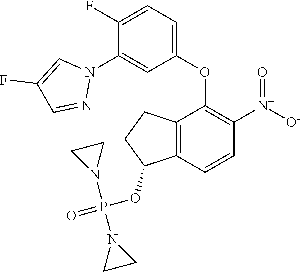 [SMILES: O=[N+]([O-])c1ccc2c(c1Oc1ccc(F)c(-n3cc(F)cn3)c1)CC[C@H]2OP(=O)(N1CC1)N1CC1] | LCMS (ESI): m/z 504.1 [M + H]⁺; ¹H NMR (400 MHz, CDCl₃) δ 7.97-7.93 (m, 1H), 7.91 (d, J = 8.2 Hz, 1H), 7.58-7.53 (m, 2H), 7.47-7.42 (m, 1H), 7.20-7.13 (m, 1H), 6.79-6.73 (m, 1H), 6.05-5.98 (m, 1H), 2.96-2.86 (m, 1H), 2.71-2.55 (m, 2H), 2.33- 2.19 (m, 9H). ee: 96% Retention time: 1.823 min (Method 36). |
| 421 | 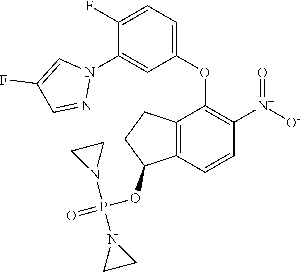 [SMILES: O=[N+]([O-])c1ccc2c(c1Oc1ccc(F)c(-n3cc(F)cn3)c1)CC[C@@H]2OP(=O)(N1CC1)N1CC1] | LCMS (ESI): m/z 504.1 [M + H]⁺; ¹H NMR (400 MHz, CDCl₃) δ 7.97-7.93 (m, 1H), 7.91 (d, J = 8.2 Hz, 1H), 7.58-7.53 (m, 2H), 7.47-7.43 (m, 1H), 7.20-7.13 (m, 1H), 6.80-6.72 (m, 1H), 6.06-5.97 (m, 1H), 2.97-2.86 (m, 1H), 2.72-2.55 (m, 2H), 2.30- 2.18 (m, 9H). ee: 99% Retention time: 2.272 min (Method 36). |
| 422 | 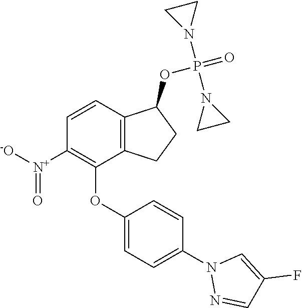 | LCMS (ESI): m/z 486.1 [M + H]⁺; ¹H NMR (400 MHz, CDCl₃) δ 7.91 (d, J = 8.2 Hz, 1H), 7.73 (d, J = 4.8 Hz, 1H), 7.63-7.48 (m, 4H), 6.96 (d, J = 9.0 Hz, 2H), 6.01 (dd, J = 12.2, 7.0 Hz, 1H), 2.99-2.79 (m, 1H), 2.72-2.51 (m, 2H), 2.33-2.06 (m, 9H). ee: >99% Retention time: 1.475 min (Method 2). |
| 423 | 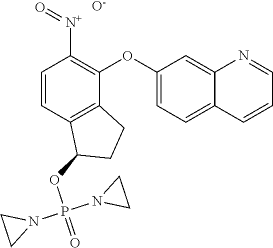 | LCMS (ESI): m/z 453.0 [M + H]⁺; ¹H NMR (400 MHz, CDCl₃) δ 8.82 (d, J = 3.3 Hz, 1H), 8.15 (d, J = 8.1 Hz, 1H), 7.99 (d, J = 8.2 Hz, 1H), 7.84 (d, J = 8.9 Hz, 1H), 7.59 (d, J = 8.2 Hz, 1H), 7.44-7.37 (m, 1H), 7.35-7.29 (m, 1H), 7.20 (d, J = 2.1 Hz, 1H), 6.09-5.97 (m, 1H), 2.99-2.90 (m, 1H), 2.74-2.53 (m, 2H), 2.29-2.16 (m, 9H). ee: 98% Retention time: 6.004 min (Method 39). |
| 424 | 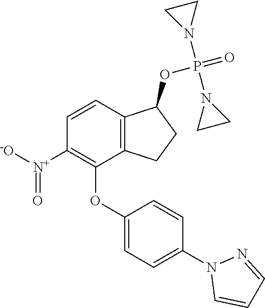 | LCMS (ESI): m/z 468.0 [M + H]⁺; ¹H NMR (400 MHz, CDCl₃) δ 7.90 (d, J = 8.2 Hz, 1H), 7.85 (d, J = 2.2 Hz, 1H), 7.71 (s, 1H), 7.62 (d, J = 8.9 Hz, 2H), 7.53 (d, J = 8.2 Hz, 1H), 6.96 (d, J = 8.9 Hz, 2H), 6.46 (s, 1H), 6.08-5.94 (m, 1H), 2.96-2.80 (m, 1H), 2.67-2.49 (m, 2H), 2.30-2.14 (m, 9H). ee: >99% Retention time: 1.675 min (Method 43). |
| 425 | 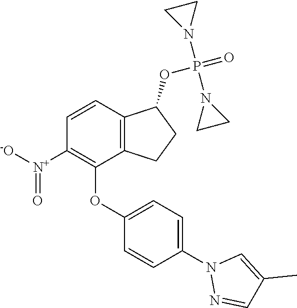 | LCMS (ESI): m/z 482.2 [M + H]⁺; ¹H NMR (400 MHz, CDCl₃) δ 7.89 (d, J = 8.2 Hz, 1H), 7.64-7.50 (m, 5H), 6.94 (d, J = 9.0 Hz, 2H), 6.05-5.84 (m, 1H), 2.92-2.80 (m, 1H), 2.65-2.51 (m, 2H), 2.28-2.18 (m, 9H), 2.16 (s, 3H). ee: >99% Retention time: 2.525 min (Method 12). |
| 426 | 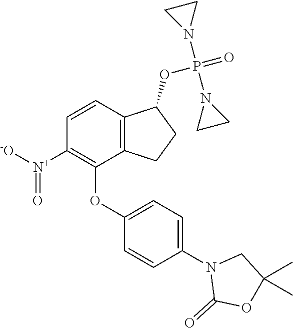 | LCMS (ESI): m/z 515.1 [M + H]⁺; ¹H NMR (400 MHz, CDCl₃) δ 7.87 (d, J = 8.2 Hz, 1H), 7.50 (d, J = 8.2 Hz, 1H), 7.48-7.43 (m, 2H), 6.91-6.85 (m, 2H), 6.03-5.95 (m, 1H), 3.74 (s, 2H), 2.89-2.78 (m, 1H), 2.65-2.49 (m, 2H), 2.29-2.16 (m, 9H), 1.55 (s, 6H). ee: 98% Retention time: 4.335 min (Method 37). |
| 427 | 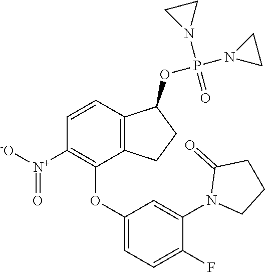 | LCMS (ESI): m/z 503.1 [M + H]⁺; ¹H NMR (400 MHz, CDCl₃) δ 7.87 (d, J = 8.2 Hz, 1H), 7.52 (d, J = 8.2 Hz, 1H), 7.10-7.03 (m, 1H), 7.00-6.96 (m, 1H), 6.78-6.71 (m, 1H), 6.03- 5.96 (m, 1H), 3.92-3.76 (m, 2H), 2.96-2.86 (m, 1H), 2.73-2.56 (m, 2H), 2.55-2.47 (m, 2H), 2.31-2.15 (m, 11H). ee: 95% Retention time: 6.615 min (Method 27). |
| 428 | 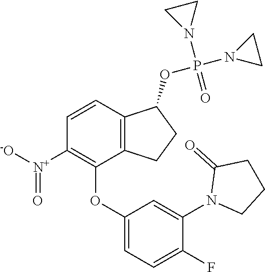 | LCMS (ESI): m/z 503.1 [M + H]⁺; ¹H NMR (400 MHz, CDCl₃) δ 7.87 (d, J = 8.2 Hz, 1H), 7.52 (d, J = 8.2 Hz, 1H), 7.11-7.03 (m, 1H), 7.01-6.97 (m, 1H), 6.77-6.70 (m, 1H), 6.04- 5.95 (m, 1H), 3.89-3.77 (m, 2H), 2.96-2.85 (m, 1H), 2.73-2.55 (m, 2H), 2.55-2.48 (m, 2H), 2.30-2.14 (m, 11H). ee: 95% Retention time: 6.064 min (Method 27). |
| 429 | 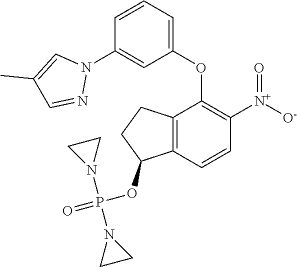 [SMILES: Cc1cnn(-c2cccc(Oc3c([N+](=O)[O-])ccc4c3CC[C@@H]4OP(=O)(N3CC3)N3CC3)c2)c1] | LCMS (ESI): m/z 482.2 [M + H]⁺; ¹H NMR (400 MHz, CDCl₃) δ 7.91 (d, J = 8.2 Hz, 1H), 7.67 (s, 1H), 7.53 (d, J = 8.2 Hz, 1H), 7.50 (s, 1H), 7.34 (d, J = 5.1 Hz, 2H), 7.27 (s, 1H), 6.75-6.67 (m, 1H), 6.03-5.98 (m, 1H), 2.94-2.87 (m, 1H), 2.71-2.50 (m, 2H), 2.31-2.17 (m, 9H), 2.15 (s, 3H). ee: >99% Retention time: 4.687 min (Method 47). |
| 430 | 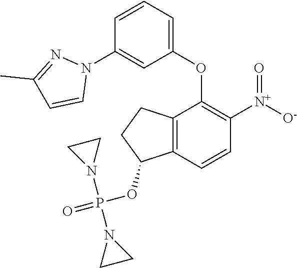 [SMILES: Cc1ccn(-c2cccc(Oc3c([N+](=O)[O-])ccc4c3CC[C@H]4OP(=O)(N3CC3)N3CC3)c2)n1] | LCMS (ESI): m/z 482.2 [M + H]⁺; ¹H NMR (400 MHz, DMSO) δ 8.39 (d, J = 2.4 Hz, 1H), 8.06 (d, J = 8.2 Hz, 1H), 7.64-7.52 (m, 2H), 7.45-7.37 (m, 2H), 6.74 (dd, J = 8.1, 2.3 Hz, 1H), 6.34 (d, J = 2.4 Hz, 1H), 5.97-5.89 (m, 1H), 2.82-2.55 (m, 3H), 2.24 (s, 3H), 2.17-2.07 (m, 9H). ee: >99% Retention time: 2.970 min (Method 47). |
| 431 | 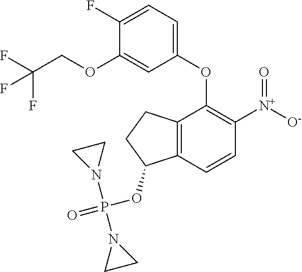 [SMILES: O=[N+]([O-])c1ccc2c(c1Oc1ccc(F)c(OCC(F)(F)F)c1)CC[C@H]2OP(=O)(N1CC1)N1CC1] | LCMS (ESI): m/z 518.1 [M + H]⁺; ¹H NMR (400 MHz, CDCl₃) δ 7.89 (d, J = 8.2 Hz, 1H), 7.54 (d, J = 8.2 Hz, 1H), 7.03 (t, J = 9.8 Hz, 1H), 6.65 (dd, J = 6.6, 2.6 Hz, 1H), 6.49- 6.41 (m, 1H), 6.01 (t, J = 6.3 Hz, 1H), 4.41 (q, J = 8.1 Hz, 2H), 2.91- 2.81 (m, 1H), 2.66-2.51 (m, 2H), 2.32-2.15 (m, 9H). ee: 98% Retention time: 1.241 min (Method 48). |
| 432 | 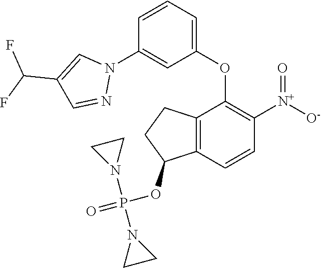 [SMILES: O=[N+]([O-])c1ccc2c(c1Oc1cccc(-n3cc(C(F)F)cn3)c1)CC[C@@H]2OP(=O)(N1CC1)N1CC1] | LCMS (ESI): m/z 518.1 [M + H]⁺; ¹H NMR (400 MHz, CDCl₃) δ 8.07 (s, 1H), 7.93 (d, J = 8.2 Hz, 1H), 7.81 (s, 1H), 7.56 (d, J = 8.2 Hz, 1H), 7.42-7.35 (m, 2H), 7.32-7.28 (m, 1H), 6.92-6.63 (m, 2H), 6.06-5.96 (m, 1H), 2.96-2.88 (m, 1H), 2.71-2.53 (m, 2H), 2.34-2.11 (m, 9H). ee: 95% Retention time: 3.271 min (Method 1). |
| 433 | 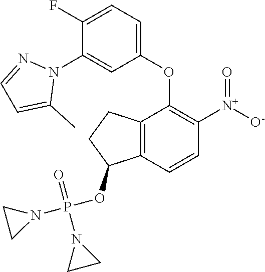 [SMILES: Cc1ccnn1-c1cc(Oc2c([N+](=O)[O-])ccc3c2CC[C@@H]3OP(=O)(N2CC2)N2CC2)ccc1F] | LCMS (ESI): m/z 500.1 [M + H]⁺; ¹H NMR (400 MHz, CDCl₃) δ 7.95-7.88 (m, 2H), 7.56-7.49 (m, 2H), 7.16-7.08 (m, 1H), 6.68-6.61 (m, 1H), 6.30-6.21 (m, 1H), 6.05-5.97 (m, 1H), 2.96-2.86 (m, 1H), 2.72-2.51 (m, 2H), 2.34 (s, 3H), 2.29-2.16 (m, 9H). ee: 95% Retention time: 2.251 min (Method 49). |
| 434 | 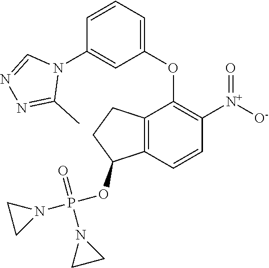 [SMILES: Cc1nncn1-c1cccc(Oc2c([N+](=O)[O-])ccc3c2CC[C@@H]3OP(=O)(N2CC2)N2CC2)c1] | LCMS (ESI): m/z 483.1 [M + H]⁺; ¹H NMR (400 MHz, CDCl₃) δ 8.42 (s, 1H), 7.94 (d, J = 8.2 Hz, 1H), 7.57 (d, J = 8.2 Hz, 1H), 7.43-7.37 (m, 1H), 7.36-7.32 (m, 1H), 7.28- 7.27 (m, 1H), 6.82-6.76 (m, 1H), 6.07-5.98 (m, 1H), 2.97-2.86 (m, 1H), 2.71-2.53 (m, 2H), 2.48 (s, 3H), 2.31-2.15 (m, 9H). ee: >99% Retention time: 4.619 min (Method 9). |
| 435 | 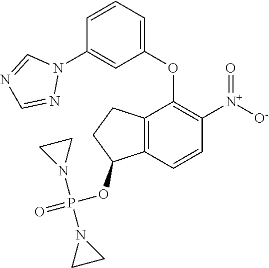 [SMILES: O=[N+]([O-])c1ccc2c(c1Oc1cccc(-n3cncn3)c1)CC[C@@H]2OP(=O)(N1CC1)N1CC1] | LCMS (ESI): m/z 469.1 [M + H]⁺; ¹H NMR (400 MHz, CDCl₃) δ 8.56 (d, J = 4.4 Hz, 1H), 8.07 (d, J = 4.8 Hz, 1H), 7.98-7.86 (m, 1H), 7.60-7.50 (m, 1H), 7.48-7.34 (m, 2H), 7.30 (t, J = 2.1 Hz, 1H), 6.90-6.77 (m, 1H), 6.11-5.89 (m, 1H), 3.01-2.88 (m, 1H), 2.75-2.50 (m, 3H), 2.34-2.12 (m, 9H). ee: >99% Retention time: 2.294 min (Method 32). |
| 436 | 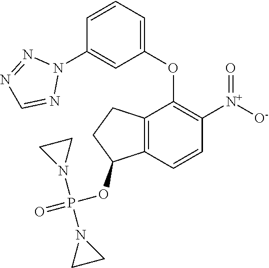 [SMILES: O=[N+]([O-])c1ccc2c(c1Oc1cccc(-n3ncnn3)c1)CC[C@@H]2OP(=O)(N1CC1)N1CC1] | LCMS (ESI): m/z 470.1 [M + H]⁺; ¹H NMR (400 MHz, CDCl₃) δ 8.64 (s, 1H), 7.96 (d, J = 8.2 Hz, 1H), 7.92-7.86 (m, 1H), 7.70-7.64 (m, 1H), 7.59 (d, J = 8.2 Hz, 1H), 7.54- 7.47 (m, 1H), 7.05-6.96 (m, 1H), 6.10-5.91 (m, 1H), 2.99-2.89 (m, 1H), 2.73-2.54 (m, 2H), 2.36-2.15 (m, 9H). ee: 99% Retention time: 4.428 min (Method 27). |
| 437 | 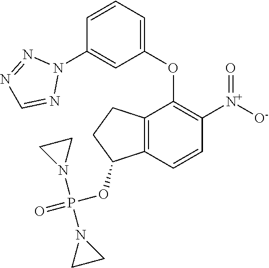 [SMILES: O=[N+]([O-])c1ccc2c(c1Oc1cccc(-n3ncnn3)c1)CC[C@H]2OP(=O)(N1CC1)N1CC1] | LCMS (ESI): m/z 470.1 [M + H]⁺; ¹H NMR (400 MHz, CDCl₃) δ 8.64 (s, 1H), 7.96 (d, J = 8.2 Hz, 1H), 7.92-7.85 (m, 1H), 7.73-7.65 (m, 1H), 7.60 (d, J = 8.2 Hz, 1H), 7.55- 7.48 (m, 1H), 7.05-6.96 (m, 1H), 6.10-5.92 (m, 1H), 3.00-2.88 (m, 1H), 2.73-2.55 (m, 2H), 2.36-2.12 (m, 9H). ee: 85% Retention time: 4.113 min (Method 27). |
| 438 | 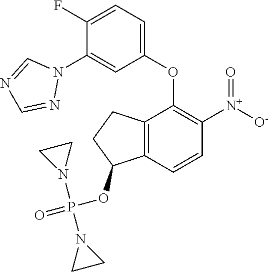 [SMILES: O=[N+]([O-])c1ccc2c(c1Oc1ccc(F)c(-n3cncn3)c1)CC[C@@H]2OP(=O)(N1CC1)N1CC1] | LCMS (ESI): m/z 487.1 [M + H]⁺; ¹H NMR (400 MHz, CDCl₃) δ 8.71 (d, J = 2.7 Hz, 1H), 8.08 (s, 1H), 7.94 (d, J = 8.2 Hz, 1H), 7.58 (d, J = 8.2 Hz, 1H), 7.50-7.40 (m, 1H), 7.23 (d, J = 10.6 Hz, 1H), 6.90-6.79 (m, 1H), 6.11-5.94 (m, 1H), 3.00- 2.88 (m, 1H), 2.72-2.55 (m, 2H), 2.36-2.11 (m, 9H). ee: 98% Retention time: 2.101 min (Method 54). |
| 439 | 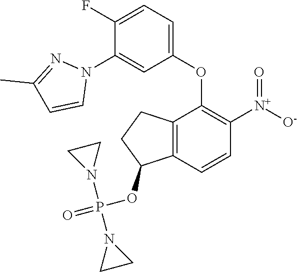 [SMILES: Cc1ccn(-c2cc(Oc3c([N+](=O)[O-])ccc4c3CC[C@@H]4OP(=O)(N3CC3)N3CC3)ccc2F)n1] | LCMS (ESI): m/z 500.2 [M + H]⁺; ¹H NMR (400 MHz, CDCl₃) δ 7.92 (dd, J = 10.0, 5.4 Hz, 2H), 7.52 (dd, J = 10.1, 5.8 Hz, 2H), 7.12 (dd, J = 11.1, 9.1 Hz, 1H), 6.69-6.62 (m, 1H), 6.26 (d, J = 2.3 Hz, 1H), 6.01 (dd, J = 12.2, 6.9 Hz, 1H), 2.96-2.85 (m, 1H), 2.70-2.52 (m, 2H), 2.34 (s, 3H), 2.32-2.13 (m, 9H). ee: 98% Retention time: 2.327 min (Method 49). |
| 440 | 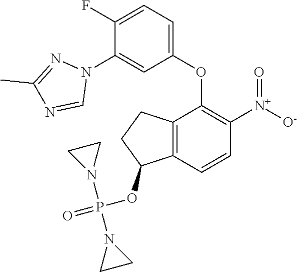 [SMILES: Cc1ncn(-c2cc(Oc3c([N+](=O)[O-])ccc4c3CC[C@@H]4OP(=O)(N3CC3)N3CC3)ccc2F)n1] | LCMS (ESI): m/z 501.2 [M + H]⁺; ¹H NMR (400 MHz, CDCl₃) δ 8.58 (d, J = 2.7 Hz, 1H), 7.96-7.90 (m, 1H), 7.57 (d, J = 8.2 Hz, 1H), 7.46 (dd, J = 6.1, 3.1 Hz, 1H), 7.24-7.16 (m, 1H), 6.81-6.75 (m, 1H), 6.02 (dd, J = 12.3, 7.0 Hz, 1H), 2.97-2.86 (m, 1H), 2.71-2.56 (m, 2H), 2.47 (s, 3H), 2.34-2.14 (m, 9H). ee: >99% Retention time: 3.856 min (Method 21). |
| 441 | 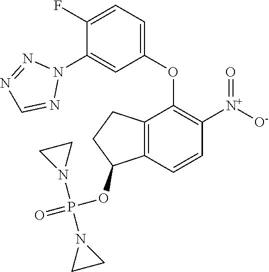 [SMILES: O=[N+]([O-])c1ccc2c(c1Oc1ccc(F)c(-n3ncnn3)c1)CC[C@@H]2OP(=O)(N1CC1)N1CC1] | LCMS (ESI): m/z 488.2 [M + H]⁺; ¹H NMR (400 MHz, CDCl₃) δ 8.71 (s, 1H), 7.95 (d, J = 8.2 Hz, 1H), 7.59 (d, J = 8.2 Hz, 1H), 7.42-7.37 (m, 1H), 7.37-7.30 (m, 1H), 7.09- 7.02 (m, 1H), 6.07-5.99 (m, 1H), 3.00-2.88 (m, 1H), 2.74-2.56 (m, 2H), 2.34-2.16 (m, 9H). ee: >99% Retention time: 3.122 min (Method 47). |
| 442 | 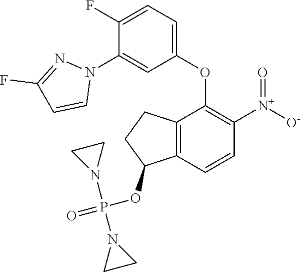 [SMILES: O=[N+]([O-])c1ccc2c(c1Oc1ccc(F)c(-n3ccc(F)n3)c1)CC[C@@H]2OP(=O)(N1CC1)N1CC1] | LCMS (ESI): m/z 504.1 [M + H]⁺; ¹H NMR (400 MHz, CDCl₃) δ 7.95-7.85 (m, 2H), 7.56 (d, J = 8.2 Hz, 1H), 7.41-7.32 (m, 1H), 7.19-7.12 (m, 1H), 6.79-6.71 (m, 1H), 6.09-5.96 (m, 2H), 2.96-2.86 (m, 1H), 2.70-2.52 (m, 2H), 2.31-2.16 (m, 9H). ee: 98% Retention time: 3.078 min (Method 22). |
| 443 | 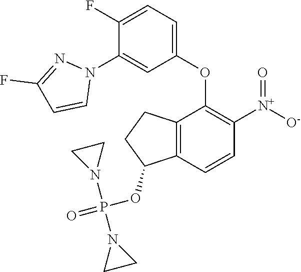 [SMILES: O=[N+]([O-])c1ccc2c(c1Oc1ccc(F)c(-n3ccc(F)n3)c1)CC[C@H]2OP(=O)(N1CC1)N1CC1] | LCMS (ESI): m/z 504.1 [M + H]⁺; ¹H NMR (400 MHz, CDCl₃) δ 7.96-7.85 (m, 2H), 7.56 (d, J = 8.2 Hz, 1H), 7.40-7.32 (m, 1H), 7.19-7.12 (m, 1H), 6.79-6.69 (m, 1H), 6.08-5.94 (m, 2H), 2.96-2.86 (m, 1H), 2.70-2.52 (m, 2H), 2.32-2.14 (m, 9H). ee: >99% Retention time: 2.500 min (Method 22). |
| 444 | 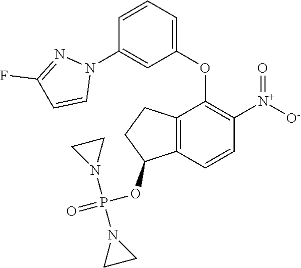 [SMILES: O=[N+]([O-])c1ccc2c(c1Oc1cccc(-n3ccc(F)n3)c1)CC[C@@H]2OP(=O)(N1CC1)N1CC1] | LCMS (ESI): m/z 486.1 [M + H]⁺; ¹H NMR (400 MHz, CDCl₃) δ 7.92 (d, J = 8.2 Hz, 1H), 7.74 (t, J = 2.5 Hz, 1H), 7.55 (d, J = 8.2 Hz, 1H), 7.35 (t, J = 8.1 Hz, 1H), 7.31-7.27 (m, 1H), 7.22 (t, J = 2.2 Hz, 1H), 6.75-6.71 (m, 1H), 6.05-5.98 (m, 2H), 2.96-2.85 (m, 1H), 2.70-2.51 (m, 2H), 2.30-2.17 (m, 9H). ee: 98% Retention time: 2.249 min (Method 32). |
| 445 | 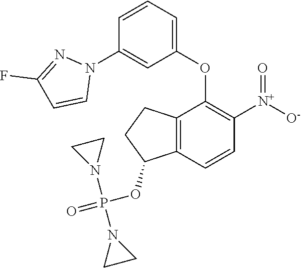 [SMILES: O=[N+]([O-])c1ccc2c(c1Oc1cccc(-n3ccc(F)n3)c1)CC[C@H]2OP(=O)(N1CC1)N1CC1] | LCMS (ESI): m/z 486.1 [M + H]⁺; ¹H 7NMR (400 MHz, CDCl₃) δ 7.92 (d, J = 8.2 Hz, 1H), 7.74 (t, J = 2.5 Hz, 1H), 7.55 (d, J = 8.2 Hz, 1H), 7.35 (t, J = 8.1 Hz, 1H), 7.31-7.27 (m, 1H), 7.22 (t, J = 2.2 Hz, 1H), 6.76-6.71 (m, 1H), 6.04-5.98 (m, 2H), 2.95-2.86 (m, 1H), 2.70-2.52 (m, 2H), 2.30-2.17 (m, 9H). ee: >99% Retention time: 2.049 min (Method 32). |

### Illustration 48. Synthesis of (S)-4-(3-(cyclopropylmethoxy)-5-hydroxyphenoxy)-5-nitro-2,3-dihydro-1H-inden-1-yl di(aziridin-1-yl)phosphinate (446)

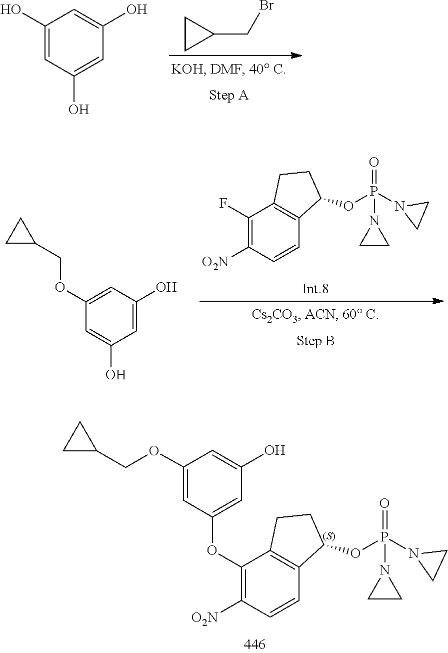

### STEP A: 5-(cyclopropylmethoxy)benzene-1,3-diol

To a solution of benzene-1,3,5-triol (3 g, 23.8 mmol, 1.0 eq.) and KOH (2.67 g, 47.6 mmol, 2.0 eq.) in DMF (20 mL) was added (bromomethyl)cyclopropane (4.8 g, 35.7 mmol, 1.5 eq.). The resulting mixture was stirred at 40° C. overnight. After completion, the reaction mixture was concentrated under reduced pressure. The residue was purified by flash column chromatography on silica gel to afford 5-(cyclopropylmethoxy)benzene-1,3-diol (200 mg, 1.11 mmol, 5%) as a white solid. LC-MS (ESI): m/z 181 [M+H]⁺.

### STEP B: (S)-4-(3-(cyclopropylmethoxy)-5-hydroxyphenoxy)-5-nitro-2,3-dihydro-1H-inden-1-yl di(aziridin-1-yl)phosphinate

To a solution of (S)-4-fluoro-5-nitro-2,3-dihydro-1H-inden-1-yl di(aziridin-1-yl)phosphinate (20 mg, 61 μmol, 1.0 eq.) and 5-(cyclopropylmethoxy)benzene-1,3-diol (55 mg, 0.31 mmol, 5.0 eq.) in ACN (1 mL) was added Cs₂CO₃ (40 mg, 0.12 mmol, 2.0 eq.). The resulting mixture was stirred at 60° C. for 1 hr. After completion, the reaction mixture was concentrated under reduced pressure. The residue was purified by prep-HPLC to give (S)-4-(3-(cyclopropylmethoxy)-5-hydroxyphenoxy)-5-nitro-2,3-dihydro-1H-inden-1-yl di(aziridin-1-yl)phosphinate 446 (9.2 mg, 19 μmol, 31%) as yellow solid. LC-MS (ESI): m/z 488.2 [M+H]⁺; Retention time: 3.580 min, 98% ee. Analytical method: Column: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C. ¹H NMR (400 MHz, CDCl₃) δ 7.86 (d, J=8.2 Hz, 1H), 7.50-7.43 (m, 1H), 6.13-6.10 (m, 1H), 6.03-6.01 (m, 1H), 5.99-5.93 (m, 2H), 5.73 (s, 1H), 3.71 (d, J=7.0 Hz, 2H), 2.96-2.87 (m, 1H), 2.69-2.50 (m, 2H), 2.29-2.15 (m, 9H), 1.24-1.18 (m, 1H), 0.65-0.60 (m, 2H), 0.34-0.30 (m, 2H). ³¹P NMR (162 MHz, CDCl₃) δ 29.85 (s).

TABLE 13
Characterization of compounds (see Illustration 48)

| Compound | | |
|---|---|---|
| No. | Structure | LCMS; ¹H NMR (ppm); Retention time |
| 447 | 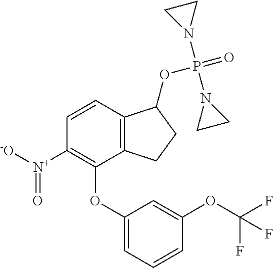 | LCMS(ESI): m/z 508.1 [M + Na]⁺; ¹H NMR (400 MHz, CDCl₃) δ7.92 (d, J = 8.2 Hz, 1H), 7.56 (d, J = 8.2 Hz, 1H), 7.32 (t, J = 8.2 Hz, 1H), 6.98-6.93 (m, 1H), 6.82-6.73 (m, 2H), 6.04-5.98 (m, 1H), 2.94-2.81 (m, 1H), 2.68-2.52 (m, 2H), 2.33-2.13 (m, 9H). |
| 448 | 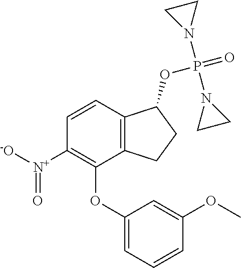 | LCMS(ESI): m/z 454.1 [M + Na]⁺; ¹H NMR (400 MHz, CDCl₃) δ7.88 (d, J = 8.2 Hz, 1H), 7.50 (d, J = 8.2 Hz, 1H), 7.18 (t, J = 8.2 Hz, 1H), 6.62 (dd, J = 8.1, 2.1 Hz, 1H), 6.47 (t, J = 2.3 Hz, 1H), 6.44-6.38 (m, 1H), 6.04-5.95 (m, 1H), 3.78 (s, 3H), 2.94-2.81 (m, 1H), 2.69-2.49 (m, 2H), 2.29-2.15 (m, 9H). ee: >99% Retention time: 2.559 min (Method 1). |
| 449 | 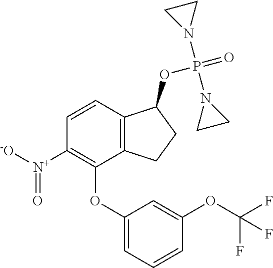 | LCMS(ESI): m/z 508.1 [M + Na]⁺; ¹H NMR (400 MHz, CDCl₃) δ 7.92 (d, J = 8.2 Hz, 1H), 7.56 (d, J = 8.2 Hz, 1H), 7.34-7.28 (m, 1H), 6.98-6.93 (m, 1H), 6.80-6.73 (m, 2H), 6.06- 5.97 (m, 1H), 2.92-2.82 (m, 1H), 2.68-2.52 (m, 2H), 2.30-2.15 (m, 9H). ee: >99% Retention time: 1.084 min (Method 18). |
| 450 | 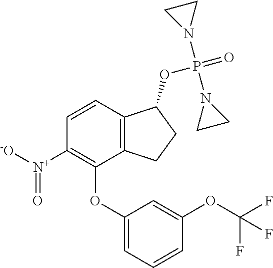 | LCMS(ESI): m/z 508.1 [M + Na]⁺; ¹H NMR (400 MHz, CDCl₃) δ 7.92 (d, J = 8.2 Hz, 1H), 7.56 (d, J = 8.2 Hz, 1H), 7.35-7.29 (m, 1H), 6.98-6.92 (m, 1H), 6.80-6.74 (m, 2H), 6.06- 5.96 (m, 1H), 2.91-2.81 (m, 1H), 2.69-2.52 (m, 2H), 2.29-2.15 (m, 9H). ee: >99% Retention time: 1.287 min (Method 18). |
| 451 | 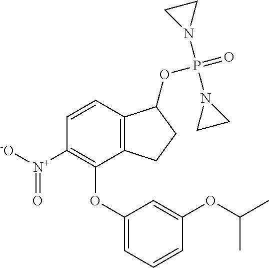 | LCMS(ESI): m/z 482.2 [M + Na]⁺; ¹H NMR (400 MHz, CDCl₃) δ 7.87 (d, J = 8.2 Hz, 1H), 7.48 (t, J = 7.1 Hz, 1H), 7.16 (t, J = 8.2 Hz, 1H), 6.60 (dd, J = 8.2, 2.0 Hz, 1H), 6.45- 6.35 (m, 2H), 6.02-5.95 (m, 1H), 4.57-4.45 (m, 1H), 2.92-2.82 (m, 1H), 2.67-2.48 (m, 2H), 2.29-2.12 (m, 9H), 1.32 (d, J = 6.1 Hz, 6H). |
| 452 | 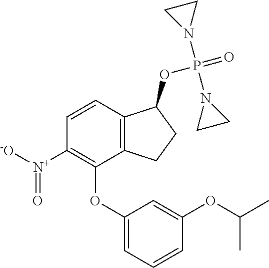 | LCMS(ESI): m/z 460.2 [M + H]⁺; ¹H NMR (400 MHz, CDCl₃) δ 7.87 (d, J = 8.2 Hz, 1H), 7.53-7.45 (m, 1H), 7.16 (t, J = 8.2 Hz, 1H), 6.60 (dd, J = 8.2, 2.0 Hz, 1H), 6.45-6.35 (m, 2H), 6.03-5.95 (m, 1H), 4.55-4.44 (m, 1H), 2.93-2.83 (m, 1H), 2.67- 2.50 (m, 2H), 2.29-2.14 (m, 9H), 1.32 (d, J = 6.1 Hz, 6H). ee: >99% Retention time: 1.660 min (Method 22). |
| 453 | 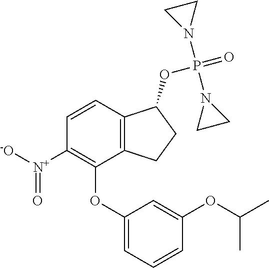 | LCMS(ESI): m/z 460.2 [M + H]⁺; ¹H NMR (400 MHz, CDCl₃) δ 7.87 (d, J = 8.2 Hz, 1H), 7.49 (d, J = 8.2 Hz, 1H), 7.16 (t, J = 8.2 Hz, 1H), 6.60 (dd, J = 8.3, 2.0 Hz, 1H), 6.46- 6.37 (m, 2H), 6.03-5.96 (m, 1H), 4.56-4.44 (m, 1H), 2.94-2.80 (m, 1H), 2.67-2.48 (m, 2H), 2.31-2.15 (m, 9H), 1.32 (d, J = 6.1 Hz, 6H). ee: >99% Retention time: 1.846 min (Method 22). |
| 454 | 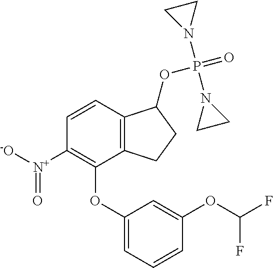 | LCMS(ESI): m/z 490.1 [M + Na]⁺; ¹H NMR (400 MHz, CDCl₃) δ 7.91 (d, J = 8.2 Hz, 1H), 7.54 (d, J = 8.2 Hz, 1H), 7.31-7.27 (m, 1H), 6.87-6.82 (m, 1H), 6.72-6.30 (m, 3H), 6.05- 5.97 (m, 1H), 2.94-2.82 (m, 1H), 2.68-2.52 (m, 2H), 2.29-2.15 (m, 9H). |
| 455 | 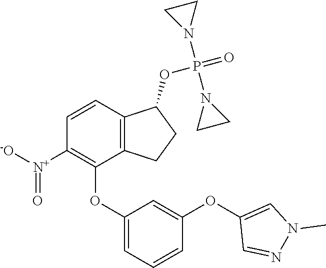 | LCMS(ESI): m/z 498.1 [M + H]⁺; ¹H NMR (400 MHz, CDCl₃) δ 7.88 (d, J = 8.2 Hz, 1H), 7.51 (d, J = 8.2 Hz, 1H), 7.34-7.31 (m, 1H), 7.27-7.24 (m, 1H), 7.19-7.14 (m, 1H), 6.74- 6.67 (m, 1H), 6.61-6.58 (m, 1H), 6.50-6.45 (m, 1H), 6.04-5.96 (m, 1H), 3.88 (s, 3H), 2.95-2.84 (m, 1H), 2.69-2.50 (m, 2H), 2.29-2.14 (m, 9H). ee: >99% Retention time: 5.952 min (Method 1). |
| 456 | 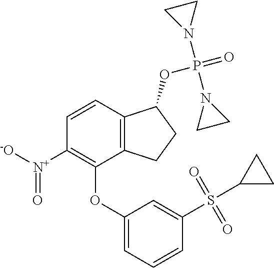 | LCMS(ESI): m/z 506.1 [M + H]⁺; ¹H NMR (400 MHz, CDCl₃) δ 7.93 (d, J = 8.2 Hz, 1H), 7.66-7.44 (m, 3H), 7.39-7.33 (m, 1H), 7.16 (dd, J = 8.1, 2.4 Hz, 1H), 6.08-5.93 (m, 1H), 2.94-2.79 (m, 1H), 2.70-2.52 (m, 2H), 2.49-2.38 (m, 1H), 2.32-2.13 (m, 9H), 1.37-1.28 (m, 2H), 1.11-0.99 (m, 2H). ee: 97% Retention time: 4.156 min (Method 1). |
| 457 | 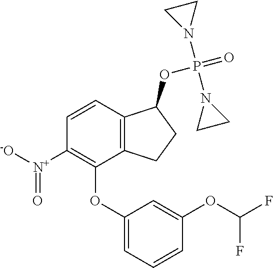 | LCMS(ESI): m/z 490.1 [M + Na]⁺; ¹H NMR (400 MHz, CDCl₃) δ 7.91 (d, J = 8.2 Hz, 1H), 7.54 (d, J = 8.2 Hz, 1H), 7.28-7.23 (m, 1H), 6.87-6.81 (m, 1H), 6.74-6.30 (m, 3H), 6.05- 5.95 (m, 1H), 2.96-2.82 (m, 1H), 2.68-2.52 (m, 2H), 2.31-2.14 (m, 9H). ee: >99% Retention time: 2.091 min (Method 23). |
| 458 | 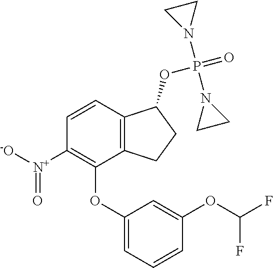 | LCMS(ESI): m/z 490.1 [M + Na]⁺; ¹H NMR (400 MHz, CDCl₃) δ 7.91 (d, J = 8.2 Hz, 1H), 7.54 (d, J = 8.2 Hz, 1H), 7.28-7.23 (m, 1H), 6.87-6.81 (m, 1H), 6.74-6.30 (m, 3H), 6.05- 5.95 (m, 1H), 2.96-2.82 (m, 1H), 2.68-2.52 (m, 2H), 2.31-2.14 (m, 9H). ee: >99% Retention time: 2.405 min (Method 23). |
| 459 | 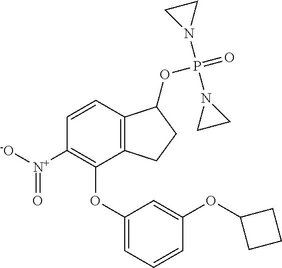 | LCMS(ESI): m/z 472.1 [M + H]⁺; ¹H NMR (400 MHz, CDCl₃) δ 7.87 (d, J = 8.2 Hz, 1H), 7.59-7.43 (m, 1H), 7.15 (t, J = 8.2 Hz, 1H), 6.52 (dd, J = 8.0, 1.9 Hz, 1H), 6.42 (dd, J = 8.2, 2.2 Hz, 1H), 6.36-6.32 (m, 1H), 6.05-5.83 (m, 1H), 4.63-4.54 (m, 1H), 3.69-3.55 (m, 1H), 2.93-2.81 (m, 1H), 2.67-2.49 (m, 2H), 2.45-2.35 (m, 2H), 2.30-2.08 (m, 10H), 1.89-1.78 (m, 1H), 1.73-1.62 (m, 1H). |
| 460 | 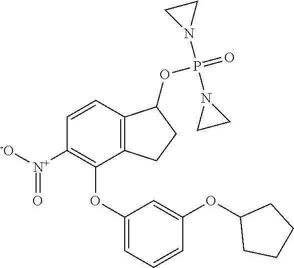 | LCMS(ESI): m/z 486.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 8.2 Hz, 1H), 6.62-6.55 (m, 1H), 6.43-6.36 (m, 2H), 6.06-5.92 (m, 1H), 4.75-4.66 (m, 1H), 2.94-2.81 (m, 1H), 2.68-2.48 (m, 2H), 2.32-2.13 (m, 9H), 1.91-1.74 (m, 6H), 1.65-1.58 (m, 2H). |
| 461 | 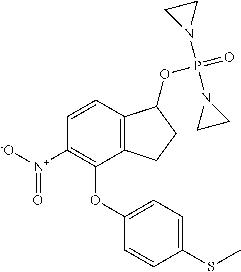 | LCMS(ESI): m/z 448.0 [M + H]⁺; ¹H NMR (400 MHz, CDCl₃) δ7.80 (d, J = 8.2 Hz, 1H), 7.43 (d, J = 8.2 Hz, 1H), 7.18-7.13 (m, 2H), 6.78-6.72 (m, 2H), 5.96-5.85 (m, 1H), 2.84- 2.71 (m, 1H), 2.58-2.44 (m, 2H), 2.39 (s, 3H), 2.24-2.06 (m, 9H). |
| 462 | 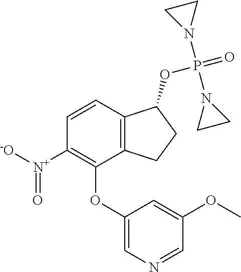 | LCMS(ESI): m/z 433.1 [M + H]⁺; ¹H NMR (400 MHz, CDCl₃) δ 8.01 (d, J = 2.4 Hz, 1H), 7.86 (d, J = 8.2 Hz, 1H), 7.77 (d, J = 2.4 Hz, 1H), 7.50 (d, J = 8.2 Hz, 1H), 6.72-6.66 (m, 1H), 5.98-5.90 (m, 1H), 3.78 (s, 3H), 2.89-2.79 (m, 1H), 2.63-2.48 (m, 2H), 2.21-2.12 (m, 9H). ee: >99% Retention time: 3.785 min (Method 24). |
| 463 | 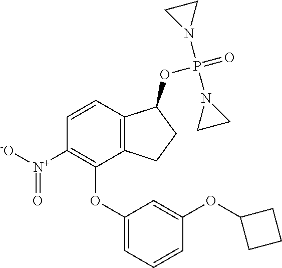 | LCMS(ESI): m/z 472.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 8.2 Hz, 1H), 6.56-6.48 (m, 1H), 6.45-6.39 (m, 1H), 6.34 (t, J = 2.3 Hz, 1H), 6.03-5.95 (m, 1H), 4.63-4.54 (m, 1H), 2.92-2.81 (m, 1H), 2.67-2.49 (m, 2H), 2.45-2.35 (m, 2H), 2.28-2.09 (m, 11H), 1.90-1.80 (m, 1H), 1.73-1.62 (m, 1H). ee: >99% Retention time: 2.236 min (Method 1). |
| 464 | 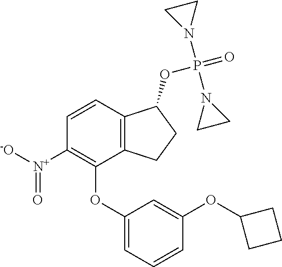 | LCMS(ESI): m/z 472.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 8.2 Hz, 1H), 6.55-6.49 (m, 1H), 6.45-6.40 (m, 1H), 6.34 (t, J = 2.3 Hz, 1H), 6.03-5.95 (m, 1H), 4.63-4.55 (m, 1H), 2.93-2.83 (m, 1H), 2.67-2.49 (m, 2H), 2.45-2.35 (m, 2H), 2.29-2.08 (m, 11H), 1.90-1.79 (m, 1H), 1.73-1.62 (m, 1H). ee: >99% Retention time: 2.759 min (Method 1). |
| 465 | 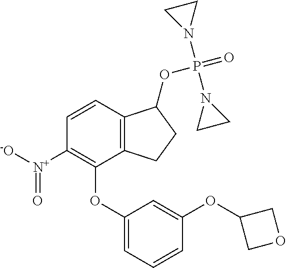 | LCMS(ESI): m/z 474.1[M + H]⁺; ¹H NMR (400 MHz, CDCl₃) δ 7.89 (d, J = 8.2 Hz, 1H), 7.52 (d, J = 8.2 Hz, 1H), 7.17 (t, J = 8.3 Hz, 1H), 6.49-6.41 (m, 1H), 6.39-6.33 (m, 1H), 6.30 (t, J = 2.3 Hz, 1H), 6.09-5.93 (m, 1H), 5.21-5.13 (m, 1H), 4.94 (t, J = 6.9 Hz, 2H), 4.77-4.71 (m, 2H), 2.94-2.82 (m, 1H), 2.69-2.51 (m, 2H), 2.29-2.16 (m, 9H). |
| 466 | 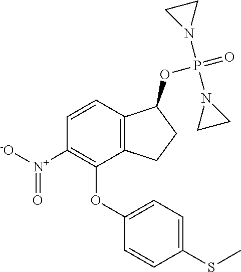 | LCMS(ESI): m/z 448.1 [M + H]⁺; ¹H NMR (400 MHz, CDCl₃) δ 7.87 (d, J = 8.2 Hz, 1H), 7.50 (d, J = 8.2 Hz, 1H), 7.25-7.21 (m, 2H), 6.84-6.80 (m, 2H), 6.03-5.94 (m, 1H), 2.89- 2.80 (m, 1H), 2.63-2.52 (m, 2H), 2.46 (s, 3H), 2.28-2.18 (m, 9H). ee: >99% Retention time: 3.554 min (Method 12). |
| 467 | 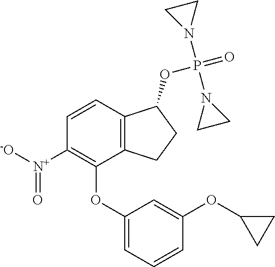 | LCMS(ESI): m/z 458.2 [M + H]⁺; ¹H NMR (400 MHz, CDCl₃) δ 7.88 (d, J = 8.2 Hz, 1H), 7.49 (d, J = 8.2 Hz, 1H), 7.20-7.16 (m, 1H), 6.79 (dd, J = 8.2, 2.2 Hz, 1H), 6 .. 60-6.56 (m, 1H), 6.43 (dd, J = 8.2, 2.3 Hz, 1H), 6.02-5.97 (m, 1H), 3.79-3.63 (m, 1H), 2.91-2.85 (m, 1H), 2.74-2.48 (m, 2H), 2.37-2.10 (m, 9H), 0.76 (d, J = 4.5 Hz, 4H). ee: >99% Retention time: 2.508 min (Method 1). |
| 468 | 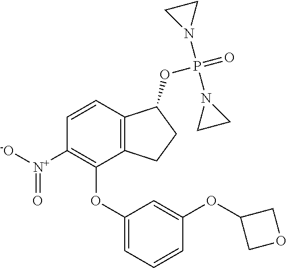 | LCMS(ESI): m/z 474.2 [M + H]⁺; ¹H NMR (400 MHz, CDCl₃) δ 7.89 (d, J = 8.2 Hz, 1H), 7.52 (d, J = 8.2 Hz, 1H), 7.17 (t, J = 8.2 Hz, 1H), 6.50-6.40 (m, 1H), 6.39-6.33 (m, 1H), 6.30 (t, J = 2.3 Hz, 1H), 6.05-5.96 (m, 1H), 5.22-5.13 (m, 1H), 4.94 (t, J = 6.8 Hz, 2H), 4.77-4.71 (m, 2H), 2.96-2.81 (m, 1H), 2.69-2.51 (m, 2H), 2.31-2.15 (m, 9H). ee: 99% Retention time: 4.136 min (Method 1). |
| 469 | 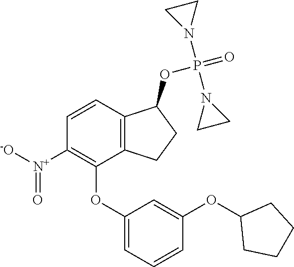 | LCMS(ESI): m/z 486.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 8.2 Hz, 1H), 6.65-6.54 (m, 1H), 6.44-6.35 (m, 2H), 6.07-5.89 (m, 1H), 4.80-4.60 (m, 1H), 2.94-2.81 (m, 1H), 2.68-2.49 (m, 2H), 2.32-2.12 (m, 9H), 1.93-1.75 (m, 6H), 1.70-1.61 (m, 2H). ee: 97% Retention time: 2.342 min (Method 21). |
| 470 | 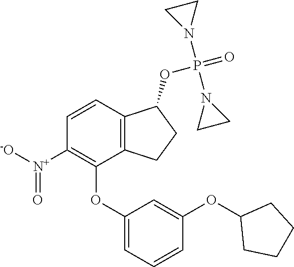 | LCMS(ESI): m/z 486.2 [M + H]⁺; ¹H NMR (400 MHz, CDCl₃) δ 7.87 (d, J = 8.2 Hz, 1H), 7.49 (d, J = 8.2 Hz, 1H), 7.15 (t, J = 8.2 Hz, 1H), 6.65-6.54 (m, 1H), 6.44-6.35 (m, 2H), 6.07-5.89 (m, 1H), 4.80-4.60 (m, 1H), 2.94-2.81 (m, 1H), 2.68-2.49 (m, 2H), 2.32-2.12 (m, 9H), 1.93-1.75 (m, 6H), 1.70-1.61 (m, 2H). ee: 98% Retention time: 2.872 min (Method 21). |
| 471 | 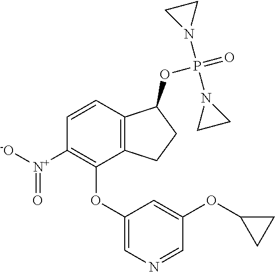 | LCMS(ESI): m/z 459.2 [M + H]⁺; ¹H NMR (400 MHz, CDCl₃) δ 8.20 (d, J = 2.2 Hz, 1H), 7.92 (t, J = 6.2 Hz, 1H), 7.86 (d, J = 2.3 Hz, 1H), 7.56 (d, J = 8.2 Hz, 1H), 6.92-6.89 (m, 1H), 6.04-5.99 (m, 1H), 3.85-3.70 (m, 1H), 2.90-2.89 (m, 1H), 2.76-2.52 (m, 2H), 2.37-2.13 (m, 9H), 0.82-0.76(m, 4H). ee: >99% Retention time: 1.220 min (Method 7). |
| 472 | 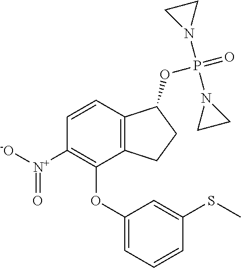 | LCMS(ESI): m/z 448.2 [M + H]⁺; ¹H NMR (400 MHz, CDCl₃) δ 7.89 (d, J = 8.2 Hz, 1H), 7.51 (d, J = 8.2 Hz, 1H), 7.19 (t, J = 8.0 Hz, 1H), 6.95 (dd, J = 7.9, 0.8 Hz, 1H), 6.82- 6.79 (m, 1H), 6.60-6.54 (m, 1H), 6.03-5.97 (m, 1H), 2.92-2.82 (m, 1H), 2.66-2.51 (m, 2H), 2.46 (s, 3H), 2.29-2.17 (m, 9H). ee: >99% Retention time: 3.259 min (Method 1). |
| 473 | 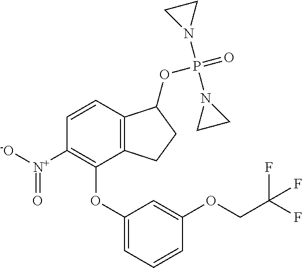 | LCMS(ESI): m/z 500.1 [M + H]⁺; ¹H NMR (400 MHz, CDCl₃) δ 7.93 (d, J = 8.2 Hz, 1H), 7.55 (d, J = 8.2 Hz, 1H), 7.25 (d, J = 8.1 Hz, 1H), 6.67 (d, J = 8.5 Hz, 1H), 6.55 (d, J = 8.0 Hz, 2H), 6.06-6.00 (m, 1H), 4.40-4.30 (m, 2H), 2.96-2.87 (m, 1H), 2.70-2.54 (m, 2H), 2.31-2.20 (m, 9H). |
| 474 | 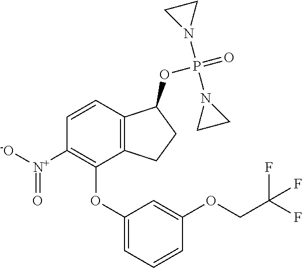 | LCMS(ESI): m/z 500.1 [M + H]⁺; ¹H NMR (400 MHz, CDCl₃) δ 7.90 (d, J = 8.4 Hz, 1H), 7.53 (d, J = 8.1 Hz, 1H), 7.22 (d, J = 7.2 Hz, 1H), 6.64 (d, J = 8.8 Hz, 1H), 6.52 (d, J = 8.1 Hz, 2H), 6.00 (s, 1H), 4.39-4.26 (m, 2H), 2.87 (s, 1H), 2.61 (t, J = 18.5 Hz, 2H), 2.31-2.13 (m, 9H). ee: >99% Retention time: 1.045 min (Method 1). |
| 475 | 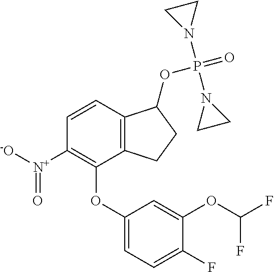 | LCMS(ESI): m/z 486 [M + H]⁺; ¹H NMR (400 MHz, CDCl₃) δ 7.90 (d, J = 7.9 Hz, 1H), 7.55 (d, J = 8.0 Hz, 1H), 7.10 (t, J = 9.7 Hz, 1H), 6.84-6.79 (m, 1H), 6.70-6.64 (m, 1H), 6.55 (t, J = 74 Hz, 1H), 6.04-5.97 (m, 1H), 2.93-2.82 (m, 1H), 2.67-2.53 (m, 2H), 2.31-2.15 (m, 9H). |
| 476 | 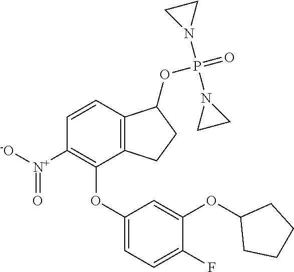 | LCMS(ESI): m/z 504.2 [M + H]⁺; ¹H NMR (400 MHz, CDCl₃) δ 7.86 (d, J = 7.7 Hz, 1H), 7.50 (d, J = 7.5 Hz, 1H), 7.27-7.24 (m, 1H), 6.94 (t, J = 9.2 Hz, 1H), 6.65-6.54 (m, 1H), 6.28-6.17 (m, 1H), 6.08-5.89 (m, 1H), 4.73 (s, 1H), 2.93-2.78 (m, 1H), 2.67-2.48 (m, 2H), 2.33-2.12 (m, 9H), 1.91-1.78 (m, 6H), 1.70-1.59 (m, 2H). |
| 477 | 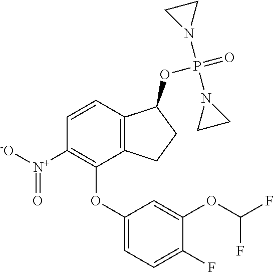 | LCMS(ESI): m/z 486.1 [M + H]⁺; ¹H NMR (400 MHz, CDCl₃) δ 7.90 (d, J = 8.2 Hz, 1H), 7.55 (d, J = 8.2 Hz, 1H), 7.10 (t, J = 9.4 Hz, 1H), 6.84-6.78 (m, 1H), 6.71-6.63 (m, 1H), 6.37-6.74 (m, 1H), 6.03-5.97 (m, 1H), 2.93-2.81 (m, 1H), 2.69-2.51 (m, 2H), 2.30-2.15 (m, 9H). ee: >99% Retention time: 1.141 min (Method 22). |
| 478 | 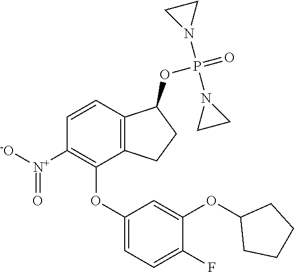 | LCMS(ESI): m/z 504.3 [M + H]⁺; ¹H NMR (400 MHz, CDCl₃) δ 7.86 (d, J = 8.2 Hz, 1H), 7.49 (d, J = 8.2 Hz, 1H), 6.97-6.89 (m, 1H), 6.63-6.57 (m, 1H), 6.27-6.18 (m, 1H), 6.07- 5.93 (m, 1H), 4.78-4.69 (m, 1H), 2.91-2.78 (m, 1H), 2.65-2.50 (m, 2H), 2.28-2.17 (m, 9H), 1.91-1.76 (m, 6H), 1.66-1.60 (m, 2H). ee: >99% Retention time: 1.068 min (Method 32). |
| 479 | 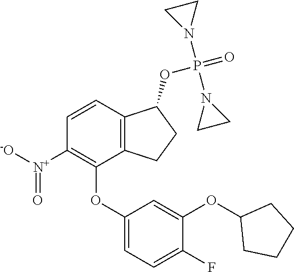 | LCMS(ESI): m/z 504.1 [M + H]⁺; ¹H NMR (400 MHz, CDCl₃) δ 7.86 (d, J = 8.2 Hz, 1H), 7.49 (d, J = 8.2 Hz, 1H), 6.97-6.90 (m, 1H), 6.63-6.57 (m, 1H), 6.28-6.20 (m, 1H), 6.04- 5.94 (m, 1H), 4.76-4.69 (m, 1H), 2.95-2.75 (m, 1H), 2.65-2.50 (m, 2H), 2.28-2.16 (m, 9H), 1.90-1.76 (m, 6H), 1.66-1.61 (m, 2H). ee: >99% Retention time: 1.283 min (Method 32). |
| 480 | 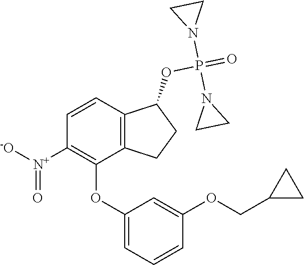 | LCMS(ESI): m/z 472.3 [M + H]⁺; ¹H NMR (400 MHz, CDCl₃) δ 7.88 (d, J = 8.2 Hz, 1H), 7.49 (d, J = 8.2 Hz, 1H), 7.20-7.14 (m, 1H), 6.63-6.58 (m, 1H), 6.45-6.41 (m, 2H), 5.99 (dd, J = 12.3, 6.8 Hz, 1H), 3.76 (d, J = 7.0 Hz, 2H), 2.93-2.82 (m, 1H), 2.68-2.50 (m, 2H), 2.30-2.14 (m, 9H), 1.28-1.19 (m, 1H), 0.69-0.58 (m, 2H), 0.38-0.29 (m, 2H). |
| 481 | 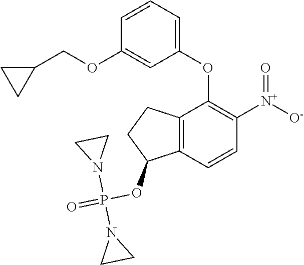 [SMILES: O=[N+]([O-])c1ccc2c(c1Oc1cccc(OCC3CC3)c1)CC[C@@H]2OP(=O)(N1CC1)N1CC1] | LCMS(ESI): m/z 472.2 [M + H]⁺; ¹H NMR (400 MHz, CDCl₃) δ 7.87 (d, J = 8.2 Hz, 1H), 7.49 (d, J = 8.3 Hz, 1H), 7.17 (t, J = 8.3 Hz, 1H), 6.60 (d, J = 8.2 Hz, 1H), 6.46-6.41 (m, 2H), 6.03-5.96 (m, 1H), 3.76 (d, J = 6.9 Hz, 2H), 2.93-2.82 (m, 1H), 2.68-2.49 (m, 2H), 2.29-2.14 (m, 9H), 1.27-1.21 (m, 1H), 0.63 (d, J = 7.3 Hz, 2H), 0.33 (d, J = 4.6 Hz, 2H). ee: 98% Retention time: 4.354 min (Method 41). |
| 482 | 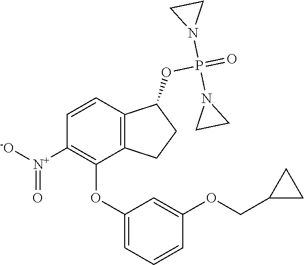 | LCMS(ESI): m/z 472.2 [M + H]⁺; ¹H NMR (400 MHz, CDCl₃) δ 7.88 (d, J = 8.2 Hz, 1H), 7.49 (d, J = 8.2 Hz, 1H), 7.20-7.13 (m, 1H), 6.63-6.57 (m, 1H), 6.45-6.41 (m, 2H), 6.03- 5.95 (m, 1H), 3.76 (d, J = 7.0 Hz, 2H), 2.93-2.83 (m, 1H), 2.68-2.49 (m, 2H), 2.28-2.15 (m, 9H), 1.28-1.20 (m, 1H), 0.63 (q, J = 5.3 Hz, 2H), 0.33 (q, J = 5.1 Hz, 2H). ee: 98% Retention time: 4.554 min (Method 41). |
| 483 | 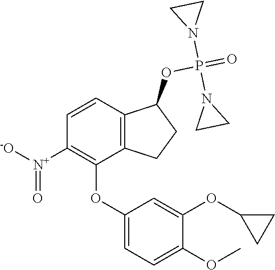 | LCMS(ESI): m/z 488.1 [M + H]⁺; ¹H NMR (400 MHz, CDCl₃) δ 7.84 (d, J = 8.2 Hz, 1H), 7.47 (d, J = 8.2 Hz, 1H), 6.99 (d, J = 2.9 Hz, 1H), 6.71 (d, J = 8.8 Hz, 1H), 6.28 (dd, J = 8.8, 2.9 Hz, 1H), 6.04-5.93 (m, 1H), 3.81 (s, 3H), 3.73-3.68 (m, 1H), 2.93-2.79 (m, 1H), 2.69-2.47 (m, 2H), 2.29-2.16 (m, 9H), 0.88-0.73 (m, 4H). ee: >99% Retention time: 2.871 min (Method 1). |
| 484 | 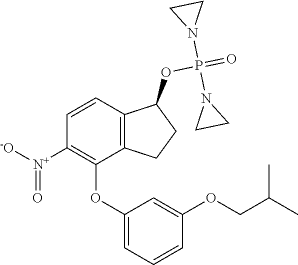 | LCMS(ESI): m/z 474.1 [M + H]⁺; ¹H NMR (400 MHz, CDCl₃) δ 7.88 (d, J = 8.2 Hz, 1H), 7.49 (d, J = 8.2 Hz, 1H), 7.19-7.12 (m, 1H), 6.63-6.58 (m, 1H), 6.48-6.43 (m, 1H), 6.41- 6.35 (m, 1H), 6.04-5.95 (m, 1H), 3.68 (d, J = 6.5 Hz, 2H), 2.94-2.80 (m, 1H), 2.68-2.48 (m, 2H), 2.30-2.18 (m, 9H), 2.10-2.01 (m, 1H), 1.01 (d, J = 6.7 Hz, 6H). ee: >99% Retention time: 1.889 min (Method 1). |
| 485 | 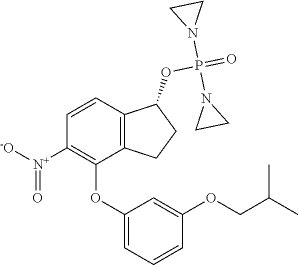 | LCMS(ESI): m/z 474.1 [M + H]⁺; ¹H NMR (400 MHz, CDCl₃) δ 7.88 (d, J = 8.2 Hz, 1H), 7.49 (d, J = 8.2 Hz, 1H), 7.19-7.11 (m, 1H), 6.65-6.56 (m, 1H), 6.50-6.43 (m, 1H), 6.42- 6.33 (m, 1H), 6.04-5.95 (m, 1H), 3.68 (d, J = 6.5 Hz, 2H), 2.94-2.83 (m, 1H), 2.68-2.50 (m, 2H), 2.29-2.17 (m, 9H), 2.10-2.00 (m, 1H), 1.01 (d, J = 6.7 Hz, 6H). ee: >99% Retention time: 1.651 min (Method 1). |
| 486 | 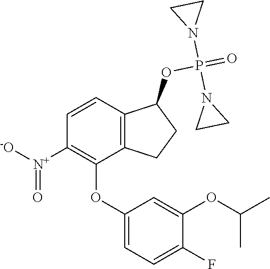 | LCMS(ESI): m/z 478.2 [M + H]⁺; ¹H NMR (400 MHz, DMSO) δ 8.01 (d, J = 8.2 Hz, 1H), 7.52 (d, J = 8.2 Hz, 1H), 7.21-7.07 (m, 1H), 6.87-6.82 (m, 1H), 6.38-6.31 (m, 1H), 5.95- 5.88 (m, 1H), 4.66-4.53 (m, 1H), 2.77-2.52 (m, 3H), 2.17-2.03 (m, 9H), 1.27-1.22 (m, 6H). ee: 96% Retention time: 1.203 min (Method 1). |
| 487 | 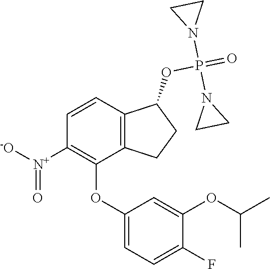 | LCMS(ESI): m/z 478.2 [M + H]⁺; ¹H NMR (400 MHz, DMSO) δ 8.01 (d, J = 8.2 Hz, 1H), 7.52 (d, J = 8.2 Hz, 1H), 7.20-7.10 (m, 1H), 6.87-6.78 (m, 1H), 6.41-6.28 (m, 1H), 5.96- 5.87 (m, 1H), 4.65-4.56 (m, 1H), 2.76-2.52 (m, 3H), 2.20-2.00 (m, 9H), 1.31-1.19 (m, 6H). ee: 95% Retention time: 1.396 min (Method 1). |
| 488 | 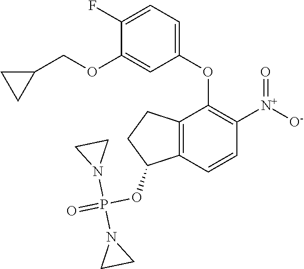 [SMILES: O=[N+]([O-])c1ccc2c(c1Oc1ccc(F)c(OCC3CC3)c1)CC[C@H]2OP(=O)(N1CC1)N1CC1] | LCMS(ESI): m/z 490.2 [M + H]⁺; ¹H NMR (400 MHz, DMSO) δ 8.01 (d, J = 8.2 Hz, 1H), 7.52 (d, J = 8.2 Hz, 1H), 7.15 (dd, J = 11.1, 8.9 Hz, 1H), 6.82 (dd, J = 7.1, 2.9 Hz, 1H), 6.36-6.28 (m, 1H), 5.97-5.85 (m, 1H), 3.90-3.80 (m, 2H), 2.76-2.62 (m, 1H), 2.59-2.51 (m, 2H), 2.20-1.99 (m, 9H), 1.28-1.16 (m, 1H), 0.62-0.53 (m, 2H), 0.36-0.25 (m, 2H). ee: >99% Retention time: 1.762 min (Method 1). |
| 489 | 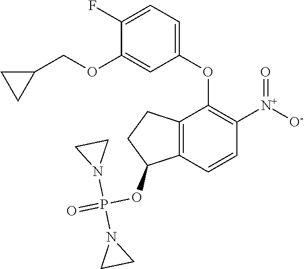 [SMILES: O=[N+]([O-])c1ccc2c(c1Oc1ccc(F)c(OCC3CC3)c1)CC[C@@H]2OP(=O)(N1CC1)N1CC1] | LCMS(ESI): m/z 490.2 [M + H]⁺; ¹H NMR (400 MHz, DMSO) δ 8.01 (d, J = 8.2 Hz, 1H), 7.52 (d, J = 8.2 Hz, 1H), 7.15 (dd, J = 11.1, 8.9 Hz, 1H), 6.82 (dd, J = 7.1, 2.9 Hz, 1H), 6.36-6.24 (m, 1H), 5.96-5.87 (m, 1H), 3.86 (d, J = 7.1 Hz, 2H), 2.75- 2.65 (m, 1H), 2.59-2.52 (m, 2H), 2.19-2.05 (m, 9H), 1.23-1.14 (m, 1H), 0.62-0.51 (m, 2H), 0.37-0.25 (m, 2H). ee: >99% Retention time: 2.419 min (Method 1). |
| 490 | 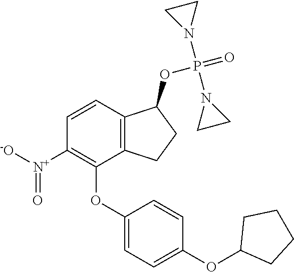 | LCMS(ESI): m/z 486.2 [M + H]⁺; ¹H NMR (400 MHz, CDCl₃) δ 7.82 (d, J = 8.2 Hz, 1H), 7.45 (d, J = 8.2 Hz, 1H), 6.82-6.76 (m, 4H), 6.02-5.92 (m, 1H), 4.71-4.65 (m, 1H), 2.86- 2.75 (m, 1H), 2.59-2.47 (m, 2H), 2.30-2.13 (m, 9H), 1.89-1.76 (m, 6H), 1.65-1.57 (m, 2H). ee: 96% Retention time: 6.106 min (Method 1). |
| 491 | 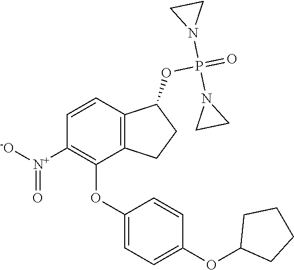 | LCMS(ESI): m/z 486.2 [M + H]⁺; ¹H NMR (400 MHz, CDCl₃) δ 7.82 (d, J = 8.2 Hz, 1H), 7.45 (d, J = 8.2 Hz, 1H), 6.82-6.76 (m, 4H), 6.02-5.92 (m, 1H), 4.72-4.65 (m, 1H), 2.86- 2.74 (m, 1H), 2.60-2.46 (m, 2H), 2.31-2.12 (m, 9H), 1.91-1.75 (m, 6H), 1.65-1.59 (m, 2H). ee: 98% Retention time: 2.970 min (Method 1). |
| 492 | 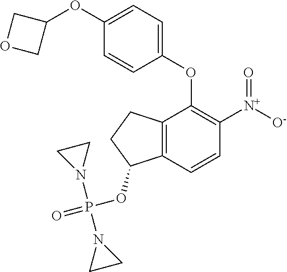 [SMILES: O=[N+]([O-])c1ccc2c(c1Oc1ccc(OC3COC3)cc1)CC[C@H]2OP(=O)(N1CC1)N1CC1] | LCMS(ESI): m/z 474.1 [M + H]⁺; ¹H NMR (400 MHz, CDCl₃) δ 7.84 (d, J = 8.2 Hz, 1H), 7.47 (d, J = 8.2 Hz, 1H), 6.84-6.78 (m, 2H), 6.68-6.58 (m, 2H), 6.04-5.93 (m, 1H), 5.18- 5.10 (m, 1H), 4.98-4.91 (m, 2H), 4.78-4.70 (m, 2H), 2.88-2.76 (m, 1H), 2.63-2.48 (m, 2H), 2.30-2.18 (m, 9H). ee: >99% Retention time: 4.199 min (Method 47). |
| 493 | 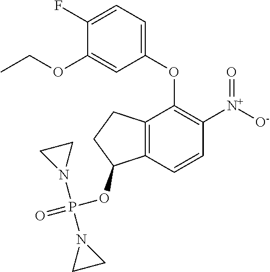 [SMILES: CCOc1cc(Oc2c([N+](=O)[O-])ccc3c2CC[C@@H]3OP(=O)(N2CC2)N2CC2)ccc1F] | LCMS(ESI): m/z 464.1 [M + H]⁺; ¹H NMR (400 MHz, CDCl₃) δ 7.85 (d, J = 8.2 Hz, 1H), 7.49 (d, J = 8.2 Hz, 1H), 6.98-6.89 (m, 1H), 6.65-6.58 (m, 1H), 6.27-6.18 (m, 1H), 6.02- 5.95 (m, 1H), 4.05 (q, J = 7.0 Hz, 2H), 2.90-2.79 (m, 1H), 2.65-2.50 (m, 2H), 2.39-2.05 (m, 9H), 1.43 (t, J = 7.0 Hz, 3H). ee: 95% Retention time: 1.995 min (Method 1). |
| 494 | 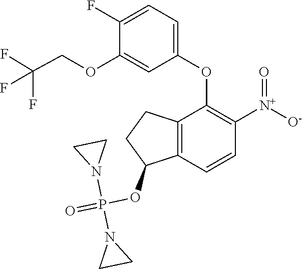 [SMILES: O=[N+]([O-])c1ccc2c(c1Oc1ccc(F)c(OCC(F)(F)F)c1)CC[C@@H]2OP(=O)(N1CC1)N1CC1] | LCMS(ESI): m/z 518.1 [M + H]⁺; ¹H NMR (400 MHz, CDCl₃) δ 7.89 (d, J = 8.2 Hz, 1H), 7.54 (d, J = 8.2 Hz, 1H), 7.03 (dd, J = 10.5, 9.1 Hz, 1H), 6.65 (dd, J = 6.7, 2.9 Hz, 1H), 6.45 (dt, J = 9.0, 3.1 Hz, 1H), 6.00 (dd, J = 12.2, 7.1 Hz, 1H), 4.41 (q, J = 8.1 Hz, 2H), 2.92-2.81 (m, 1H), 2.66-2.51 (m, 2H), 2.30-2.16 (m, 9H). ee: 95% Retention time: 1.645 min (Method 48). |
| 495 | 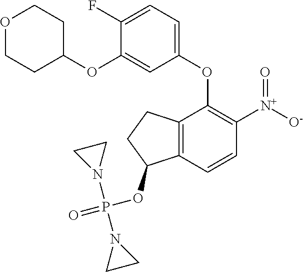 [SMILES: O=[N+]([O-])c1ccc2c(c1Oc1ccc(F)c(OC3CCOCC3)c1)CC[C@@H]2OP(=O)(N1CC1)N1CC1] | LCMS(ESI): m/z 520.2 [M + H]⁺; ¹H NMR (400 MHz, CDCl₃) δ 7.87 (d, J = 8.2 Hz, 1H), 7.51 (d, J = 8.2 Hz, 1H), 6.97 (dd, J = 10.4, 9.1 Hz, 1H), 6.65 (dd, J = 6.7, 2.9 Hz, 1H), 6.31 (dt, J = 8.8, 3.1 Hz, 1H), 6.00 (dd, J = 12.1, 7.0 Hz, 1H), 4.43 (dt, J = 11.7, 3.9 Hz, 1H), 4.03-3.94 (m, 2H), 3.61-3.50 (m, 2H), 2.92-2.81 (m, 1H), 2.67-2.51 (m, 2H), 2.30-2.16 (m, 9H), 2.05-1.97 (m, 2H), 1.86-1.76 (m, 2H). ee: >99% Retention time: 1.285 min (Method 5). |
| 496 | 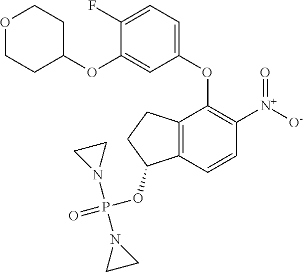 [SMILES: O=[N+]([O-])c1ccc2c(c1Oc1ccc(F)c(OC3CCOCC3)c1)CC[C@H]2OP(=O)(N1CC1)N1CC1] | LCMS(ESI): m/z 520.2 [M + H]⁺; ¹H NMR (400 MHz, CDCl₃) δ 7.87 (d, J = 8.2 Hz, 1H), 7.51 (d, J = 8.3 Hz, 1H), 6.97 (dd, J = 10.6, 9.0 Hz, 1H), 6.65 (dd, J = 6.8, 2.9 Hz, 1H), 6.31 (dt, J = 8.9, 3.1 Hz, 1H), 6.00 (dd, J = 12.1, 7.1 Hz, 1H), 4.48-4.39 (m, 1H), 4.04-3.94 (m, 2H), 3.60-3.51 (m, 2H), 2.93-2.80 (m, 1H), 2.68- 2.50 (m, 2H), 2.31-2.13 (m, 9H), 2.05-1.97 (m, 2H), 1.86-1.77 (m, 2H). ee: 94% Retention time: 1.074 min (Method 5). |
| 497 | 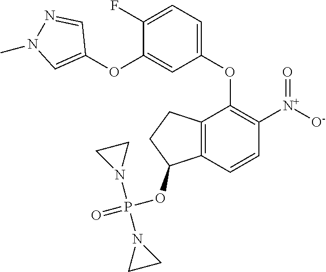 [SMILES: Cn1cc(Oc2cc(Oc3c([N+](=O)[O-])ccc4c3CC[C@@H]4OP(=O)(N3CC3)N3CC3)ccc2F)cn1] | LCMS(ESI): m/z 516.1 [M + H]⁺; ¹H NMR (400 MHz, CDCl₃) δ 7.85 (d, J = 8.2 Hz, 1H), 7.50 (d, J = 8.2 Hz, 1H), 7.34 (s, 1H), 7.28 (s, 1H), 7.07-6.99 (m, 1H), 6.72-6.65 (m, 1H), 6.40-6.32 (m, 1H), 6.03-5.94 (m, 1H), 3.88 (s, 3H), 2.91-2.79 (m, 1H), 2.66-2.51 (m, 2H), 2.29-2.15 (m, 9H). ee: 98% Retention time: 2.875 min (Method 30). |
| 498 | 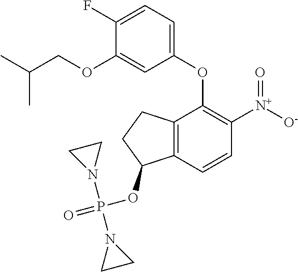 [SMILES: CC(C)COc1cc(Oc2c([N+](=O)[O-])ccc3c2CC[C@@H]3OP(=O)(N2CC2)N2CC2)ccc1F] | LCMS(ESI): m/z 492.1 [M + H]⁺; ¹H NMR (400 MHz, CDCl₃) δ 7.87 (d, J = 8.2 Hz, 1H), 7.50 (d, J = 8.2 Hz, 1H), 6.99-6.89 (m, 1H), 6.66-6.58 (m, 1H), 6.25-6.17 (m, 1H), 6.05- 5.95 (m, 1H), 3.74 (d, J = 6.6 Hz, 2H), 2.91-2.81 (m, 1H), 2.65-2.51 (m, 2H), 2.31-2.16 (m, 9H), 2.15-2.04 (m, 1H), 1.03 (d, J = 6.7 Hz, 6H). ee: 96% Retention time: 1.635 min (Method 1). |
| 499 | 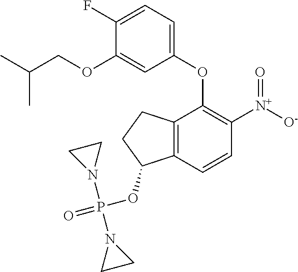 [SMILES: CC(C)COc1cc(Oc2c([N+](=O)[O-])ccc3c2CC[C@H]3OP(=O)(N2CC2)N2CC2)ccc1F] | LCMS(ESI): m/z 492.1 [M + H]⁺; ¹H NMR (400 MHz, CDCl₃) δ 7.86 (d, J = 8.2 Hz, 1H), 7.50 (d, J = 8.2 Hz, 1H), 6.97-6.88 (m, 1H), 6.66-6.58 (m, 1H), 6.25-6.17 (m, 1H), 6.04- 5.93 (m, 1H), 3.74 (d, J = 6.6 Hz, 2H), 2.91-2.79 (m, 1H), 2.65-2.49 (m, 2H), 2.30-2.15 (m, 9H), 2.15-2.07 (m, 1H), 1.03 (d, J = 6.7 Hz, 6H). ee: 95% Retention time: 1.311 min (Method 1). |
| 500 | 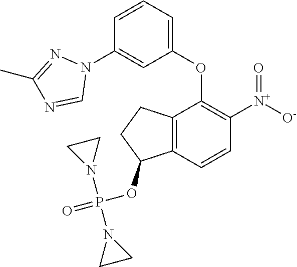 [SMILES: Cc1ncn(-c2cccc(Oc3c([N+](=O)[O-])ccc4c3CC[C@@H]4OP(=O)(N3CC3)N3CC3)c2)n1] | LCMS(ESI): m/z 483.2 [M + H]⁺; ¹H NMR (400 MHz, CDCl₃) δ 8.43-8.40 (m, 1H), 7.94 (d, J = 8.2 Hz, 1H), 7.57 (d, J = 8.2 Hz, 1H), 7.42-7.37 (m, 1H), 7.36-7.32 (m, 1H), 7.28-7.27 (m, 1H), 6.82-6.77 (m, 1H), 6.07-5.97 (m, 1H), 2.96-2.87 (m, 1H), 2.70-2.54 (m, 2H), 2.47 (s, 3H), 2.32-2.15 (m, 9H). ee: 98% Retention time: 4.813 min (Method 13). |
| 501 | 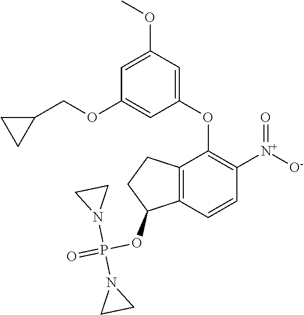 [SMILES: COc1cc(OCC2CC2)cc(Oc2c([N+](=O)[O-])ccc3c2CC[C@@H]3OP(=O)(N2CC2)N2CC2)c1] | LCMS(ESI): m/z 502.2 [M + H]⁺; ¹H NMR (400 MHz, CDCl₃) δ 7.88 (d, J = 8.2 Hz, 1H), 7.49 (d, J = 8.2 Hz, 1H), 6.20-6.14 (m, 1H), 6.06-6.02 (m, 1H), 6.02-5.95 (m, 2H), 3.75 (s, 3H), 3.72 (d, J = 7.0 Hz, 2H), 2.97-2.88 (m, 1H), 2.71-2.52 (m, 2H), 2.31-2.14 (m, 9H), 1.27-1.17 (m, 1H), 0.66-0.58 (m, 2H), 0.36-0.29 (m, 2H). ee: 95% Retention time: 3.183 min (Method 52). |
| 502 | 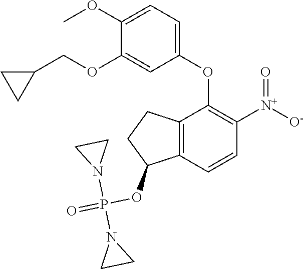 [SMILES: COc1ccc(Oc2c([N+](=O)[O-])ccc3c2CC[C@@H]3OP(=O)(N2CC2)N2CC2)cc1OCC1CC1] | LCMS(ESI): m/z 502.2 [M + H]⁺; ¹H NMR (400 MHz, CDCl₃) δ 7.83 (d, J = 8.2 Hz, 1H), 7.46 (d, J = 8.2 Hz, 1H), 6.73 (d, J = 8.8 Hz, 1H), 6.60 (d, J = 2.8 Hz, 1H), 6.28 (dd, J = 8.8, 2.8 Hz, 1H), 5.98 (dd, J = 12.1, 6.9 Hz, 1H), 3.84 (s, 3H), 3.81 (d, J = 7.0 Hz, 2H), 2.88-2.77 (m, 1H), 2.63-2.47 (m, 2H), 2.28-2.16 (m, 9H), 1.36-1.25 (m, 1H), 0.67-0.60 (m, 2H), 0.38-0.31 (m, 2H). ee: >99% Retention time: 1.656 min (Method 5). |
| 503 | 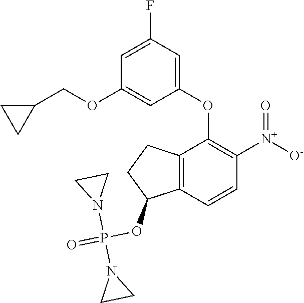 [SMILES: O=[N+]([O-])c1ccc2c(c1Oc1cc(F)cc(OCC3CC3)c1)CC[C@@H]2OP(=O)(N1CC1)N1CC1] | LCMS(ESI): m/z 490.1 [M + H]⁺; ¹H NMR (400 MHz, CDCl₃) δ 7.91 (d, J = 8.2 Hz, 1H), 7.53 (d, J = 8.2 Hz, 1H), 6.36-6.29 (m, 1H), 6.22-6.16 (m, 2H), 6.04-5.87 (m, 1H), 3.73 (d, J = 7.0 Hz, 2H), 2.97-2.88 (m, 1H), 2.71-2.54 (m, 2H), 2.32-2.13 (m, 9H), 1.26-1.18 (m, 1H), 0.67-0.61 (m, 2H), 0.36-0.31 (m, 2H). ee: 96% Retention time: 2.710 min (Method 55). |

TABLE 14
Characterization of compounds (see General Procedure 1-8)

| Compound | | |
|---|---|---|
| No. | Structure | LCMS; ¹H NMR (ppm); Retention time |
| 504 | 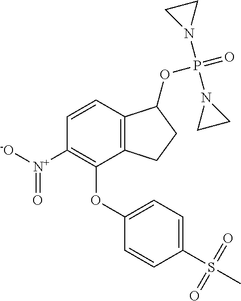 | LCMS(ESI): m/z 480.1 [M + H]⁺; ¹H NMR (400 MHz, CDCl₃) δ 7.97 (d, J = 8.2 Hz, 1H), 7.94-7.88 (m, 2H), 7.61 (d, J = 8.2 Hz, 1H), 7.05-6.96 (m, 2H), 6.05-6.02 (m, 1H), 3.07 (s, 3H), 2.97-2.82 (m, 1H), 2.73-2.53 (m, 2H), 2.36-2.14 (m, 9H). |
| 505 | 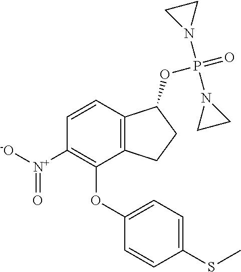 | LCMS(ESI): m/z 448.1 [M + H]⁺; ¹H NMR (400 MHz, CDCl₃) δ 7.87 (d, J = 8.2 Hz, 1H), 7.50 (d, J = 8.2 Hz, 1H), 7.25-7.21 (m, 2H), 6.83-6.79 (m, 2H), 6.04-5.95 (m, 1H), 2.88- 2.79 (m, 1H), 2.63-2.50 (m, 2H), 2.46 (s, 3H), 2.29-2.15 (m, 9H). ee: >99% Retention time: 4.429 min (Method 12). |
| 506 | 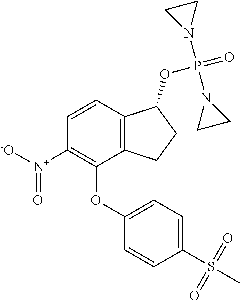 | LCMS(ESI): m/z 480.2 [M + H]⁺; ¹H NMR (400 MHz, CDCl₃) δ 7.97 (d, J = 8.2 Hz, 1H), 7.93-7.87 (m, 2H), 7.61 (d, J = 8.2 Hz, 1H), 7.06-6.97 (m, 2H), 6.06-6.01 (m, 1H), 3.07 (s, 3H), 2.94-2.84 (m, 1H), 2.70-2.57 (m, 2H), 2.31-2.18 (m, 9H). ee: >99% Retention time: 6.202 min (Method 1). |
| 507 | 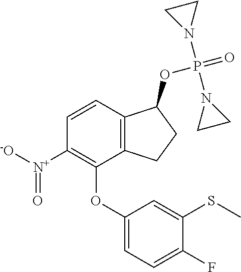 | LCMS(ESI): m/z 466.1 [M + H]* ; ¹H NMR (400 MHz, CDCl₃) δ 7.86 (d, J = 8.2 Hz, 1H), 7.50 (d, J = 8.2 Hz, 1H), 6.92 (t, J = 9.0 Hz, 1H), 6.86-6.78 (m, 1H), 6.57-6.48 (m, 1H), 6.05-5.95 (m, 1H), 2.91-2.78 (m, 1H), 2.63-2.51 (m, 2H), 2.42 (s, 3H), 2.29-2.16 (m, 9H). ee: 98% Retention time: 2.898 min (Method 1). |
| 508 | 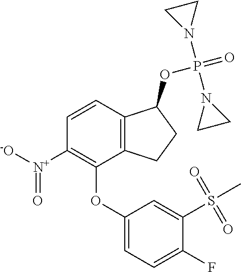 | LCMS(ESI): m/z 498.0 [M + H]⁺; ¹H NMR (400 MHz, CDCl₃) δ 7.93 (d, J = 8.2 Hz, 1H), 7.58 (d, J = 8.2 Hz, 1H), 7.39-7.35 (m, 1H), 7.22 (t, J = 8.9 Hz, 1H), 7.18-7.13 (m, 1H), 6.05-5.97 (m, 1H), 3.22 (s, 3H), 2.94-2.82 (m, 1H), 2.67-2.53 (m, 2H), 2.32-2.15 (m, 9H). ee: >99% Retention time: 2.584 min (Method 1). |
| 509 | 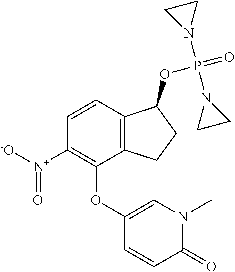 | LCMS(ESI): m/z 433.1 [M + H]⁺; ¹H NMR (400 MHz, CDCl₃) δ 7.83 (d, J = 8.2 Hz, 1H), 7.51 (d, J = 8.2 Hz, 1H), 7.21 (dd, J = 9.9, 3.2 Hz, 1H), 6.95 (d, J = 3.2 Hz, 1H), 6.57 (d, J = 9.9 Hz, 1H), 6.05-5.93 (m, 1H), 3.49 (s, 3H), 2.99-2.91 (m, 1H), 2.74- 2.55 (m, 2H), 2.32-2.16 (m, 9H). ee: >99% Retention time: 4.926 min (Method 1). |
| 510 | 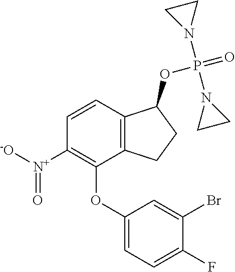 | LCMS(ESI): m/z 498.0 [M + H]⁺; ¹H NMR (400 MHz, CDCl₃) δ 7.91 (d, J = 8.2 Hz, 1H), 7.58-7.52 (m, 1H), 7.10-7.03 (m, 2H), 6.82-6.77 (m, 1H), 6.06-5.97 (m, 1H), 2.92-2.82 (m, 1H), 2.67-2.53 (m, 2H), 2.29-2.17 (m, 9H). ee: >99% Retention time: 2.233 min (Method 1). |
| 511 | 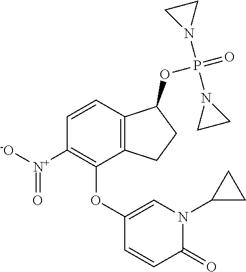 | LCMS(ESI): m/z 459.1 [M + H]⁺; ¹H NMR (400 MHz, CDCl₃) δ 7.82 (d, J = 8.2 Hz, 1H), 7.51 (d, J = 8.2 Hz, 1H), 7.16 (dd, J = 9.9, 3.2 Hz, 1H), 6.99 (d, J = 3.2 Hz, 1H), 6.53 (d, J = 9.9 Hz, 1H), 6.03-5.96 (m, 1H), 3.35-3.28 (m, 1H), 2.99-2.88 (m, 1H), 2.73-2.55 (m, 2H), 2.32-2.14 (m, 9H), 1.14-1.08 (m, 2H), 0.86-0.79 (m, 2H). . ee: 96% Retention time: 6.491 min (Method 1). |
| 512 | 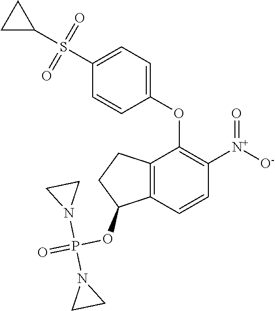 [SMILES: O=[N+]([O-])c1ccc2c(c1Oc1ccc(S(=O)(=O)C3CC3)cc1)CC[C@@H]2OP(=O)(N1CC1)N1CC1] | LCMS(ESI): m/z 506.0 [M + H]⁺; ¹H NMR (400 MHz, CDCl₃) δ 7.97 (d, J = 8.2 Hz, 1H), 7.86 (d, 2H), 7.61 (d, J = 8.2 Hz, 1H), 6.99 (d, 2H), 6.09-5.97 (m, 1H), 2.95-2.79 (m, 1H), 2.73-2.53 (m, 2H), 2.51-2.41 (m, 1H), 2.33-2.18 (m, 9H), 1.39-1.31 (m, 2H), 1.09-1.01 (m, 2H). ee: 97% Retention time: 2.313 min (Method 19). |
| 513 | 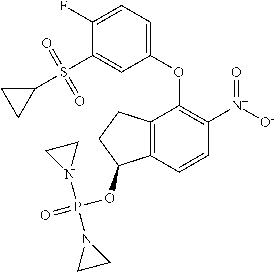 [SMILES: O=[N+]([O-])c1ccc2c(c1Oc1ccc(F)c(S(=O)(=O)C3CC3)c1)CC[C@@H]2OP(=O)(N1CC1)N1CC1] | LCMS(ESI): m/z 524.1 [M + H]⁺; ¹H NMR (400 MHz, CDCl₃) δ 7.92 (d, J = 8.2 Hz, 1H), 7.58 (d, J = 8.2 Hz, 1H), 7.35-7.30 (m, 1H), 7.21 (t, J = 8.9 Hz, 1H), 7.16-7.11 (m, 1H), 6.05-5.97 (m, 1H), 2.92-2.82 (m, 1H), 2.78-2.70 (m, 1H), 2.67-2.54 (m, 2H), 2.30-2.17 (m, 9H), 1.40-1.33 (m, 2H), 1.15-1.06 (m, 2H). ee: 97% Retention time: 3.276 min (Method 1). |
| 514 | 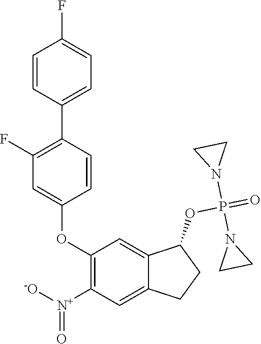 | LCMS(ESI): m/z 536.2 [M + Na]⁺; ¹H NMR (400 MHz, CDCl₃) δ 7.86 (s, 1H), 7.51-7.44 (m, 2H), 7.40-7.33 (m, 2H), 7.16-7.10 (m, 2H), 6.90-6.78 (m, 2H), 5.99-5.93 (m, 1H), 3.23- 3.13 (m, 1H), 2.98-2.88 (m, 1H), 2.69-2.60 (m, 1H), 2.37-2.27 (m, 1H), 2.24-2.07 (m, 8H). ee: 99% Retention time: 2.712 min (Method 37). |
| 515 | 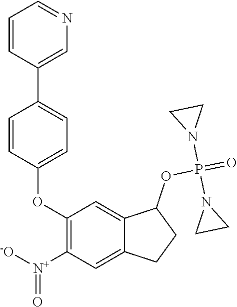 | LCMS(ESI): m/z 479.1 [M + H]⁺; ¹H NMR (400 MHz, CDCl₃) δ 8.84-8.78 (m, 1H), 8.61-8.54 (m, 1H), 7.87-7.81 (m, 2H), 7.60-7.51 (m, 2H), 7.41-7.33 (m, 1H), 7.27 (s, 1H), 7.17-7.10 (m, 2H), 5.97-5.89 (m, 1H), 3.22-3.11 (m, 1H), 2.97-2.86 (m, 1H), 2.68-2.55 (m, 1H), 2.36-2.24 (m, 1H), 2.20-1.99 (m, 8H). |
| 516 | 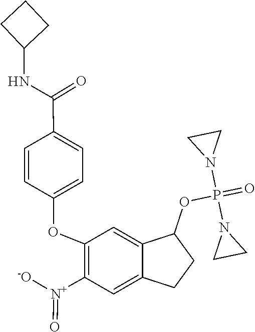 | LCMS(ESI): m/z 499.1 [M + H]⁺; ¹H NMR (400 MHz, CDCl₃) δ 7.84 (s, 1H), 7.76-7.74 (m, 2H), 7.27 (s, 1H), 7.05-6.98 (m, 2H), 6.18 (d, J = 7.2 Hz, 1H), 5.96-5.91 (m, 1H), 4.61- 4.55 (m, 1H), 3.23-3.10 (m, 1H), 2.98-2.87 (m, 1H), 2.68-2.59 (m, 1H), 2.48-2.44 (m, 2H), 2.35-2.27 (m, 1H), 2.21-2.06 (m, 8H), 1.99-1.92 (m, 2H), 1.82-1.72 (m, 2H). |
| 517 | 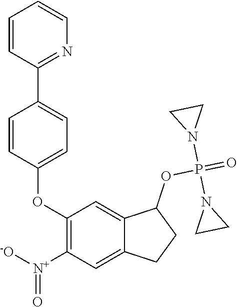 | LCMS(ESI): m/z 479.1 [M + H]⁺; ¹H NMR (400 MHz, CDCl₃) δ 8.62-8.58 (m, 1H), 7.97-7.90 (m, 2H), 7.76 (s, 1H), 7.71-7.65 (m, 1H), 7.63 (d, J = 7.9 Hz, 1H), 7.19-7.13 (m, 2H), 7.07-7.03 (m, 2H), 5.94-5.79 (m, 1H), 3.14-3.00 (m, 1H), 2.90-2.77 (m, 1H), 2.59-2.46 (m, 1H), 2.29-2.19 (m, 1H), 2.11-1.97 (m, 8H). |
| 518 | 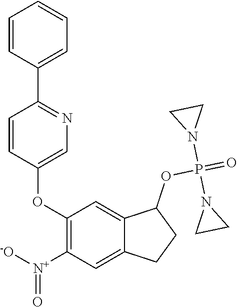 | LCMS(ESI): m/z 479.2 [M + H]⁺; ¹H NMR (400 MHz, CDCl₃) δ 8.40 (d, J = 2.8 Hz, 1H), 7.90-7.85 (m, 2H), 7.79 (s, 1H), 7.66 (d, J = 8.7 Hz, 1H), 7.43-7.38 (m, 2H), 7.36-7.31 (m, 2H), 7.22 (s, 1H), 5.94-5.77 (m, 1H), 3.15-3.04 (m, 1H), 2.93-2.79 (m, 1H), 2.62-2.50 (m, 1H), 2.28-2.19 (m, 1H), 2.08-1.97 (m, 8H). |
| 519 | 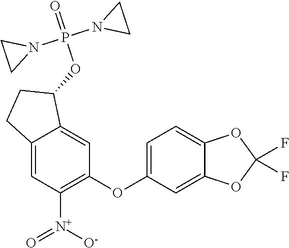 [SMILES: O=[N+]([O-])c1cc2c(cc1Oc1ccc3c(c1)OC(F)(F)O3)[C@@H](OP(=O)(N1CC1)N1CC1)CC2] | LCMS(ESI): m/z 478 [M + H]⁺; ¹H NMR (400 MHz, CDCl₃) δ 7.82 (s, 1H), 7.21 (s, 1H), 7.03 (d, J = 8.7 Hz, 1H), 6.81 (d, J = 2.4 Hz, 1H), 6.74 (dd, J = 8.7, 2.5 Hz, 1H), 5.98-5.85 (m, 1H), 3.21-3.09 (m, 1H), 2.97-2.84 (m, 1H), 2.68-2.55 (m, 1H), 2.37- 2.26 (m, 1H), 2.26-1.95 (m, 8H). ee: 95% Retention time: 5.446 min (Method 53). |
| 520 | 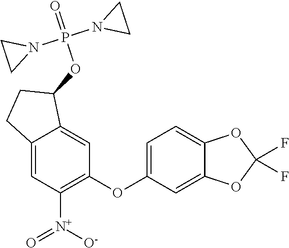 [SMILES: O=[N+]([O-])c1cc2c(cc1Oc1ccc3c(c1)OC(F)(F)O3)[C@H](OP(=O)(N1CC1)N1CC1)CC2] | LCMS(ESI): m/z 478 [M + H]⁺; ¹H NMR (400 MHz, CDCl₃) δ 7.82 (s, 1H), 7.21 (s, 1H), 7.03 (d, J = 8.7 Hz, 1H), 6.81 (d, J = 2.4 Hz, 1H), 6.74 (dd, J = 8.7, 2.5 Hz, 1H), 5.99-5.86 (m, 1H), 3.20-3.10 (m, 1H), 2.96-2.86 (m, 1H), 2.67-2.56 (m, 1H), 2.36- 2.25 (m, 1H), 2.23-2.02 (m, 8H). ee: 88% Retention time: 4.885 min (Method 53). |
| 521 | 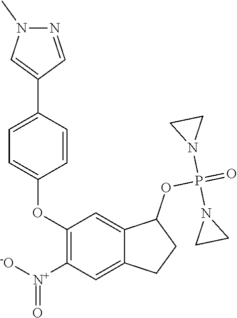 | LCMS(ESI): m/z 482.2 [M + H]⁺; ¹H NMR (400 MHz, CDCl₃) δ7.80 (s, 1H), 7.71 (s, 1H), 7.58 (s, 1H), 7.45 (d, J = 8.7 Hz, 2H), 7.18 (s, 1H), 7.04 (d, J = 8.7 Hz, 2H), 5.93-5.86 (m, 1H), 3.95 (s, 3H), 3.17-3.07 (m, 1H), 2.93-2.84 (m, 1H), 2.65-2.54 (m, 1H), 2.33-2.22 (m, 1H), 2.16-2.01 (m, 8H). ee: >99% Retention time: 10.086 min (Method 3). |
| 522 | 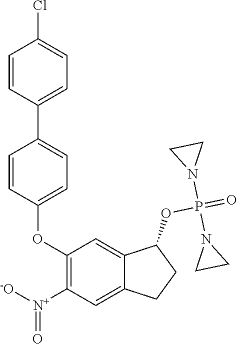 | LCMS(ESI): m/z 534.1 [M + Na]⁺; ¹H NMR (400 MHz, CDCl₃) δ 7.83 (s, 1H), 7.57-7.51 (m, 2H), 7.50-7.45 (m, 2H), 7.43-7.38 (m, 2H), 7.24 (s, 1H), 7.12-7.07 (m, 2H), 5.96-5.89 (m, 1H), 3.20-3.09 (m, 1H), 2.95-2.86 (m, 1H), 2.68-2.56 (m, 1H), 2.35-2.24 (m, 1H), 2.19-2.03 (m, 8H). ee: >99% Retention time: 4.883 min (Method 3). |
| 523 | 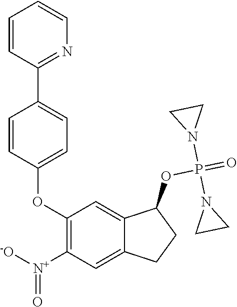 | LCMS(ESI): m/z 479.1 [M + H]⁺; ¹H NMR (400 MHz, CDCl₃) δ 8.67 (d, J = 4.4 Hz, 1H), 8.01 (d, J = 8.7 Hz, 2H), 7.83 (s, 1H), 7.79-7.72 (m, 1H), 7.69 (d, J = 7.9 Hz, 1H), 7.26- 7.19 (m, 2H), 7.12 (d, J = 8.7 Hz, 2H), 6.03-5.81 (m, 1H), 3.21-3.08 (m, 1H), 2.95-2.81 (m, 1H), 2.68-2.55 (m, 1H), 2.34-2.26 (m, 1H), 2.17-2.03 (m, 8H). ee: >99% Retention time: 5.393 min (Method 34). |
| 524 | 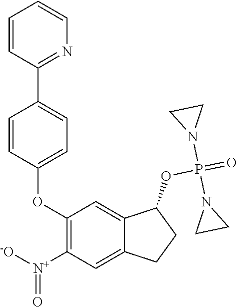 | LCMS(ESI): m/z 479.1 [M + H]⁺; ¹H NMR (400 MHz, CDCl₃) δ 8.67 (d, J = 4.7 Hz, 1H), 8.01 (d, J = 8.8 Hz, 2H), 7.83 (s, 1H), 7.78-7.72 (m, 1H), 7.70 (d, J = 7.9 Hz, 1H), 7.26- 7.20 (m, 2H), 7.12 (d, J = 8.8 Hz, 2H), 5.97-5.85 (m, 1H), 3.18-3.10 (m, 1H), 2.96-2.86 (m, 1H), 2.66-2.55 (m, 1H), 2.35-2.24 (m, 1H), 2.16-2.00 (m, 8H). ee: >99% Retention time: 3.960 min (Method 34). |
| 525 | 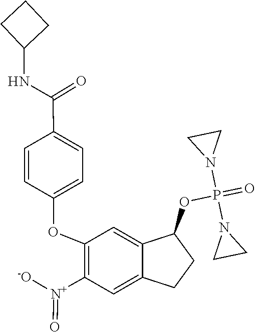 | LCMS(ESI): m/z 499.1 [M + H]⁺; ¹H NMR (400 MHz, CDCl₃) δ 7.84 (s, 1H), 7.76-7.73 (m, 2H), 7.27 (s, 1H), 7.04-6.99 (m, 2H), 6.18 (d, J = 7.6 Hz, 1H), 5.96-5.91 (m, 1H), 4.61- 4.55 (m, 1H), 3.20-3.13 (m, 1H), 2.98-2.87 (m, 1H), 2.70-2.58 (m, 1H), 2.50-2.39 (m, 2H), 2.37-2.26 (m, 1H), 2.21-2.19 (m, 1H), 2.17-2.06 (m, 7H), 2.00-1.91 (m, 2H), 1.83-1.74 (m, 2H). ee: 98% Retention time: 4.788 min (Method 1). |
| 526 | 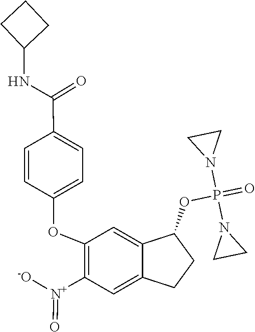 | LCMS(ESI): m/z 499.1 [M + H]⁺; ¹H NMR (400 MHz, CDCl₃) δ 7.84 (s, 1H), 7.76-7.73 (m, 2H), 7.27 (s, 1H), 7.04-6.99 (m, 2H), 6.18 (d, J = 7.6 Hz, 1H), 5.96-5.91 (m, 1H), 4.61- 4.55 (m, 1H), 3.20-3.13 (m, 1H), 2.98-2.87 (m, 1H), 2.70-2.58 (m, 1H), 2.50-2.39 (m, 2H), 2.37-2.26 (m, 1H), 2.21-2.19 (m, 1H), 2.17-2.06 (m, 7H), 2.00-1.91 (m, 2H), 1.83-1.74 (m, 2H). ee: >99% Retention time: 4.019 min (Method 1). |
| 527 | 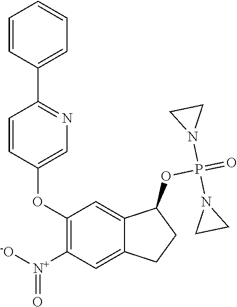 | LCMS(ESI): m/z 479.1 [M + H]⁺; ¹H NMR (400 MHz, CDCl₃) δ 8.40 (d, J = 2.8 Hz, 1H), 7.91-7.84 (m, 2H), 7.79 (s, 1H), 7.66 (d, J = 8.7 Hz, 1H), 7.40 (t, J = 7.3 Hz, 2H), 7.37- 7.31 (m, 2H), 7.22 (s, 1H), 5.87 (dd, J = 12.6, 7.2 Hz, 1H), 3.13-3.03 (m, 1H), 2.92-2.79 (m, 1H), 2.63-2.49 (m, 1H), 2.29-2.21 (m, 1H), 2.12-1.98 (m, 8H). ee: >99% Retention time: 4.976 min (Method 3). |
| 528 | 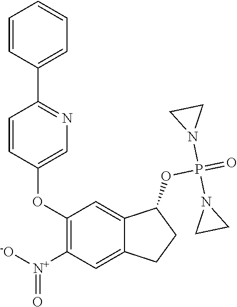 | LCMS(ESI): m/z 479.1 [M + H]⁺; ¹H NMR (400 MHz, CDCl₃) δ 8.50-8.44 (m, 1H), 7.98-7.91 (m, 2H), 7.86 (s, 1H), 7.73 (d, J = 8.6 Hz, 1H), 7.51-7.44 (m, 2H), 7.44-7.37 (m, 2H), 7.29 (s, 1H), 6.00-5.89 (m, 1H), 3.22-3.10 (m, 1H), 2.98-2.87 (m, 1H), 2.68-2.57 (m, 1H), 2.37-2.26 (m, 1H), 2.20-2.03 (m, 8H). ee: >99% Retention time: 3.853 min (Method 3). |
| 529 | 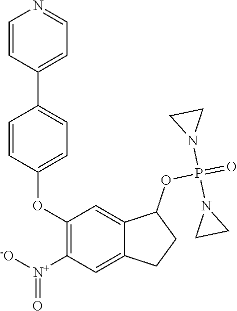 | LCMS(ESI): m/z 479.1 [M + H]⁺; ¹H NMR (400 MHz, CDCl₃) δ 8.67 (d, J = 6.2 Hz, 2H), 7.86 (s, 1H), 7.68- 7.63 (m, 2H), 7.56 (d, J = 6.0 Hz, 2H), 7.32 (s, 1H), 7.15-7.09 (m, 2H), 5.99-5.89 (m, 1H), 3.22-3.13 (m, 1H), 2.99-2.88 (m, 1H), 2.69-2.59 (m, 1H), 2.38-2.27 (m, 1H), 2.22-2.02 (m, 8H). |

### Illustration 49. Synthesis of (6-nitro-5-(3-(pyrrolidine-1-carbonyl)phenoxy)benzofuran-2-yl)methyl di(aziridin-1-yl)phosphinate (530)

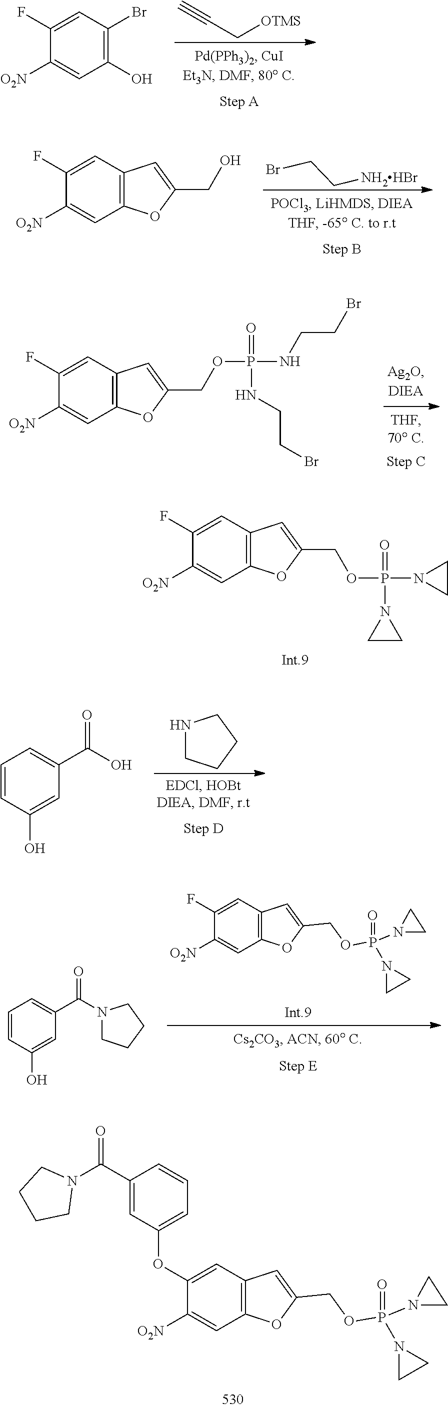

### STEP A: (5-fluoro-6-nitrobenzofuran-2-yl) methanol

To a stirred solution of 2-bromo-4-fluoro-5-nitrophenol (1.0 g, 4.26 mmol, 1.0 eq.) and trimethyl(prop-2-yn-1-yloxy) silane (1.64 g, 12.8 mmol, 3.0 eq.) in DMF (20 mL) were added Pd(PPh₃)₂Cl₂ (301 mg, 0.43 mmol, 0.1 eq.), CuI (82 mg, 0.43 mmol, 0.1 eq.) and TEA (861 mg, 8.52 mmol, 2.0 eq.) at room temperature. The resulting mixture was stirred at 80° C. for 2 hrs under nitrogen atmosphere. After completion, the reaction mixture was diluted with H₂O (20 mL) and extracted with EtOAc (20 mL×3). The combined organic layers were washed with brine (20 mL), dried over anhydrous Na₂SO₄, and concentrated under reduced pressure. The residue was purified by flash column chromatography on silica gel to afford (5-fluoro-6-nitrobenzofuran-2-yl) methanol (550 mg, 2.61 mmol, 61%) as a brown oil. LCMS (ESI): m/z 212 [M+H]⁺.

### STEP B: (5-fluoro-6-nitro-1-benzofuran-2-yl)methyl P,P-bis[(2-bromoethyl)amino]phosphinate

To a solution of (5-fluoro-6-nitrobenzofuran-2-yl) methanol (1 g, 4.74 mmol, 1.0 eq.) in THF (40 mL) was added LiHMDS (7.1 mL, 7.11 mmol, 1.5 eq., 1 M in THF) at −60° C. The resulting mixture was stirred at −60° C. for 30 min. POCl₃ (1.15 g, 7.58 mmol, 1.6 eq.) was added into the above mixture and the resulting mixture was stirred at −60° C. for 10 min. 2-bromoethan-1-amine hydrobromide (9.6 g, 47.4 mmol, 10.0 eq.) and TEA (5.74 g, 56.9 mmol, 12.0 eq.) was added into the above mixture. The resulting mixture was stirred at −60° C. for additional 30 min, then warmed up to room temperature and stirred for 1 hr. After completion, the reaction mixture was quenched with NH₄Cl (20 mL, sat. aq.) and extracted with EtOAc (20 mL×3). The combined organic layers were washed with brine (20 mL), dried over anhydrous Na₂SO₄, and concentrated under reduced pressure. The residue was purified by flash column chromatography on silica gel to afford (5-fluoro-6-nitro-1-benzofuran-2-yl)methyl P,P-bis[(2-bromoethyl)amino]phosphinate (1.5 g, 2.99 mmol, 63%) as a yellow oil. LCMS (ESI): m/z 502 [M+H]⁺.

### STEP C: (5-fluoro-6-nitrobenzofuran-2-yl)methyl di(aziridin-1-yl)phosphinate

To a solution of (5-fluoro-6-nitro-1-benzofuran-2-yl)methyl P,P-bis[(2-bromoethyl)amino]phosphinate (1.5 g, 2.99 mmol, 1.0 eq.) and DIEA (3.86 g, 29.9 mmol, 10 eq.) in THF (15 mL) was added Ag₂O (8.25 g, 35.9 mmol, 12 eq.). The resulting mixture was stirred at 70° C. overnight. After completion, the reaction mixture was cooled to room temperature and filtered through a short pad of Celite®. The filtrate was concentrated under reduced pressure, the residue was purified by prep-HPLC to give (5-fluoro-6-nitrobenzofuran-2-yl)methyl di(aziridin-1-yl)phosphinate (917 mg, 2.69 mmol, 12%) as a yellow oil. LCMS (ESI): m/z=342 [M+H]⁺.

### STEP D: (4-hydroxypyridin-2-yl) (pyrrolidin-1-yl) methanone

To a solution of 3-hydroxybenzoic acid (1 g, 7.25 mmol, 1.0 eq.), EDCI (1.39 g, 7.25 mmol, 1.0 eq.) and HOBt (979 mg, 7.25 mmol, 1.0 eq.) in DMF (20 mL) were added DIEA (2.8 g, 21.8 mmol, 3.0 eq.) and pyrrolidine (618 mg, 8.70 mmol, 1.2 eq.). The resulting mixture was stirred at 25° C. for 2 hrs. After completion, the reaction mixture was purified by prep-HPLC to give (4-hydroxypyridin-2-yl) (pyrrolidin-1-yl) methanone (400 mg, 2.08 mmol, 29%) as a yellow oil. LCMS (ESI): m/z 193 [M+H]⁺.

### STEP E: (6-nitro-5-(3-(pyrrolidine-1-carbonyl)phenoxy)benzofuran-2-yl)methyl di(aziridin-1-yl)phosphinate, 530

To a solution of (5-fluoro-6-nitrobenzofuran-2-yl)methyl di(aziridin-1-yl)phosphinate (100 mg, 0.29 mmol, 1.0 eq.) and (4-hydroxypyridin-2-yl)(pyrrolidin-1-yl)methanone (56 mg, 0.29 mmol, 1.0 eq.) in ACN (5 mL) was added Cs₂CO₃ (283 mg, 0.87 mmol, 3.0 eq.). The resulting mixture was stirred at 60° C. for 2 hrs. After completion, the reaction mixture was concentrated under reduced pressure. The residue was purified by prep-HPLC to give (6-nitro-5-(3-(pyrrolidine-1-carbonyl)phenoxy)benzofuran-2-yl)methyl di(aziridin-1-yl)phosphinate, (50 mg, 98 μmol, 33%) as a white solid. LCMS (ESI): m/z 513 [M+H]⁺. ¹H NMR (400 MHz, DMSO) δ 8.54 (s, 1H), 7.61 (s, 1H), 7.45 (t, J=8.0 Hz, 1H), 7.28 (d, J=7.6 Hz, 1H), 7.15-7.07 (m, 3H), 5.26 (d, J=8.3 Hz, 2H), 3.42 (s, 2H), 3.34 (s, 2H), 2.12 (d, J=15.4 Hz, 8H), 1.87-1.75 (m, 4H).

### Illustration 50. Synthesis of (R)-1-(6-nitro-5-(3-(trifluoromethoxy) phenoxy)benzofuran-2-yl)ethyl di(aziridin-1-yl)phosphinate (531) & (S)-1-(6-nitro-5-(3-(trifluoromethoxy) phenoxy)benzofuran-2-yl)ethyl di(aziridin-1-yl)phosphinate (532)

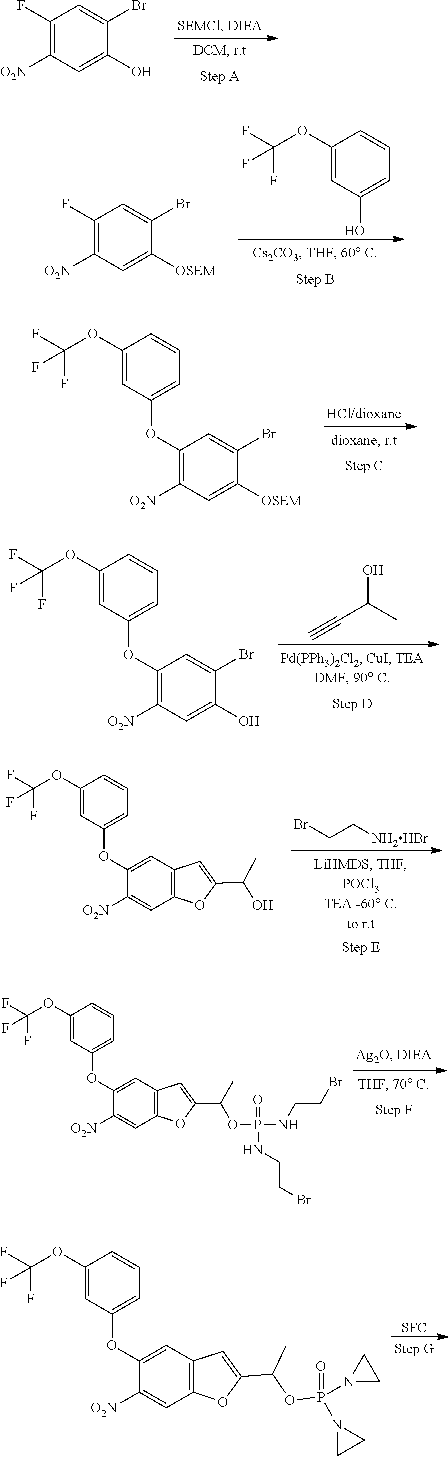

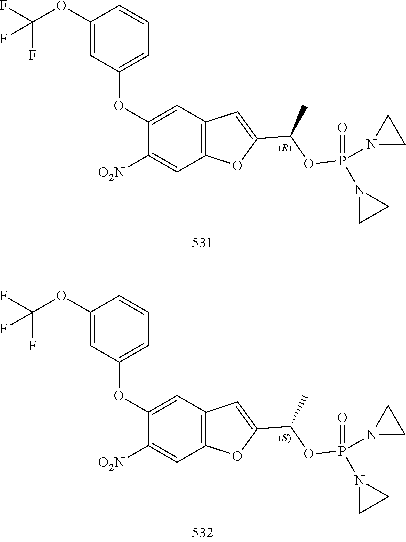

### STEP A: (2-((2-bromo-4-fluoro-5-nitrophenoxy)methoxy)ethyl)trimethylsilane

To a stirred mixture of 2-bromo-4-fluoro-5-nitrophenol (5.0 g, 21.2 mmol, 1.0 eq.) in DCM (200 mL) was added SEMCl (3.53 g, 21.2 mmol, 1.0 eq.) dropwise at room temperature. To the above mixture was added DIEA (5.48 g, 42.4 mmol, 2.0 equiv) dropwise at 0° C. The resulting mixture was stirred for additional 2 hrs at room temperature. The resulting mixture was concentrated under reduced pressure. The residue was purified by flash column chromatography on silica gel to afford [2-(2-bromo-4-fluoro-5-nitrophenoxymethoxy)ethyl]trimethylsilane (5.2 g, 14.2 mmol, 67%) as a yellow oil. LCMS (ESI): m/z 366 [M+H]⁺.

### STEP B: (2-((2-bromo-5-nitro-4-(3-(trifluoromethoxy) phenoxy) phenoxy)methoxy)ethyl)trimethylsilane

To a solution of (2-((2-bromo-4-fluoro-5 nitrophenoxy)methoxy)ethyl)trimethylsilane (1.5 g, 4.10 mmol, 1.0 eq.) and 3-(trifluoromethoxy) phenol (948 mg, 5.32 mmol, 1.3 eq.) in THF (20 mL) was added Cs₂CO₃ (2.67 g, 8.19 mmol, 2.0 eq.). The resulting mixture was stirred at 60° C. for 1 hr. After completion, the reaction mixture was concentrated under reduced pressure, the residue was purified by flash column chromatography on silica gel to afford (2-((2-bromo-5-nitro-4-(3-(trifluoromethoxy) phenoxy) phenoxy)methoxy)ethyl)trimethylsilane (2.1 g, 4.01 mmol, 98%) as a while solid. LCMS (ESI): m/z 524 [M+H]⁺.

### STEP C: 2-bromo-5-nitro-4-(3-(trifluoromethoxy) phenoxy) phenol

To a solution of (2-((2-bromo-5-nitro-4-(3-(trifluoromethoxy) phenoxy) phenoxy)methoxy)ethyl)trimethylsilane (2.1 g, 4.005 mmol, 1.0 eq.) in dioxane (5 mL) was added HCl/dioxane (5 mL, 4 M). The mixture was stirred at room temperature for 2 hrs. After completion, the reaction mixture was concentrated under reduced pressure. The residue was purified by flash column chromatography on silica gel to afford 2-bromo-5-nitro-4-(3-(trifluoromethoxy) phenoxy) phenol (1.3 g, 3.30 mmol, 82%) as a yellow oil. LCMS (ESI): m/z 393 [M+H]⁺.

### STEP D: 1-(6-nitro-5-(3-(trifluoromethoxy) phenoxy)benzofuran-2-yl) ethan-1-ol

To a stirred solution of 2-bromo-5-nitro-4-(3-(trifluoromethoxy) phenoxy) phenol (1.5 g, 3.81 mmol, 1.0 eq.) and but-3-yn-2-ol (0.8 mL, 11.4 mmol, 3.0 eq.) in DMF (20 mL) were added Pd(PPh₃)₂Cl₂ (300 mg, 0.38 mmol, 0.1 eq.), CuI (72 mg, 0.38 mmol, 0.1 eq.) and TEA (1.1 mL, 7.61 mmol, 2.0 eq.) at room temperature. The resulting mixture was stirred at 90° C. for 2 hrs under nitrogen atmosphere. After completion, the reaction mixture was diluted with H₂O (20 mL) and extracted with EtOAc (20 mL×3). The combined organic layers were washed with brine (20 mL), dried over anhydrous Na₂SO₄, and concentrated under reduced pressure. The residue was purified by flash column chromatography on silica gel to afford 1-(6-nitro-5-(3-(trifluoromethoxy) phenoxy)benzofuran-2-yl) ethan-1-ol (770 mg, 2.01 mmol, 53%) as a brown oil. ¹H NMR (400 MHz, CDCl₃) δ 8.12 (s, 1H), 7.30-7.22 (m, 2H), 6.93-6.74 (m, 4H), 6.61 (s, 1H), 5.04-4.95 (m, 1H), 1.60 (d, J=6.6 Hz, 3H).

### STEP E: 1-[6-nitro-5-({3-[(trifluoromethyl)oxy]phenyl}oxy)-1-benzofuran-2-yl]ethyl P,P-bis[(2-bromoethyl)amino]phosphinate

To a solution of 1-(6-nitro-5-(3-(trifluoromethoxy) phenoxy)benzofuran-2-yl) ethan-1-ol (300 mg, 0.78 mmol, 1.0 eq.) in THF (35 mL) was added LiHMDS (1.2 mL, 1.17 mmol, 1.5 eq., 1 M in THF) at −60° C. The resulting mixture was stirred at −60° C. for 30 min. POCl₃ (0.1 mL, 1.25 mmol, 1.6 eq.) was added into the above mixture and the resulting mixture was stirred at −60° C. for 10 min. 2-bromoethan-1-amine hydrobromide (970 mg, 7.83 mmol, 10.0 eq.) and TEA (1.3 mL, 9.39 mmol, 12.0 eq.) was added into the above mixture. The resulting mixture was stirred at −60° C. for an additional 30 min, then warmed up to room temperature and stirred for 1 hr. After completion, the reaction mixture was quenched with NH₄Cl (10 mL, sat. aq.) and extracted with EtOAc (15 mL×3). The combined organic layers were washed with brine (20 mL), dried over anhydrous Na₂SO₄, and concentrated under reduced pressure. The residue was purified by flash column chromatography on silica gel to afford 1-[6-nitro-5-({3-[(trifluoromethyl)oxy]phenyl}oxy)-1-benzofuran-2-yl]ethyl P,P-bis[(2-bromoethyl)amino]phosphinate (280 mg, 0.42 mmol, 53%) as a yellow oil. LCMS (ESI): m/z 674 [M+H]⁺.

### STEP F: 1-(6-nitro-5-(3-(trifluoromethoxy) phenoxy)benzofuran-2-yl)ethyl di(aziridin-1-yl)phosphinate

To a solution of 1-[6-nitro-5-({3-[(trifluoromethyl)oxy]phenyl}oxy)-1-benzofuran-2-yl]ethyl P,P-bis[(2-bromoethyl)amino]phosphinate (220 mg, 0.32 mmol, 1.0 eq.) and DIEA (0.7 mL, 4.15 mmol, 13 eq.) in THF (15 mL) was added Ag₂O (891 mg, 3.85 mmol, 12 eq.). The resulting mixture was stirred at 70° C. overnight. After completion, the reaction mixture was cooled to room temperature and filtered through a short pad of Celite®. The filtrate was concentrated under reduced pressure, the residue was purified by prep-HPLC to give 1-(6-nitro-5-(3-(trifluoromethoxy) phenoxy)benzofuran-2-yl)ethyl di(aziridin-1-yl)phosphinate (20 mg, 38 μmol, 12%) as a yellow oil. LCMS (ESI): m/z 514 [M+H]⁺.

### STEP G: (R)-1-(6-nitro-5-(3-(trifluoromethoxy) phenoxy)benzofuran-2-yl)ethyl di(aziridin-1-yl)phosphinate & (S)-1-(6-nitro-5-(3-(trifluoromethoxy) phenoxy)benzofuran-2-yl)ethyl di(aziridin-1-yl)phosphinate (531 & 532)

1-(6-nitro-5-(3-(trifluoromethoxy) phenoxy)benzofuran-2-yl)ethyl di(aziridin-1-yl)phosphinate (200 mg, 0.39 mmol) was further separated by Chiral SFC to give:

Enantiomer I, (R)-1-(6-nitro-5-(3-(trifluoromethoxy) phenoxy)benzofuran-2-yl)ethyl di(aziridin-1-yl)phosphinate (531) (80 mg, 40%); Retention time: 1.247 min, >99% ee. LC-MS (ESI): m/z 514 [M+H]⁺; ¹H NMR (400 MHz, CDCl₃) δ 8.21 (s, 1H), 7.39-7.30 (m, 2H), 6.99 (d, J=8.3 Hz, 1H), 6.92-6.83 (m, 2H), 6.78 (s, 1H), 5.83-5.75 (m, 1H), 2.29-2.15 (m, 8H), 1.78 (d, J=6.6 Hz, 3H). ¹⁹F NMR (376 MHz, CDCl₃) δ −57.82 (s). ³¹P NMR (162 MHz, CDCl₃) δ 30.44 (s).

Enantiomer II, (S)-1-(6-nitro-5-(3-(trifluoromethoxy) phenoxy)benzofuran-2-yl)ethyl di(aziridin-1-yl)phosphinate (532) (80 mg, 40%); Retention time: 1.475 min, >99% ee. LC-MS (ESI): m/z 514 [M+H]⁺; ¹H NMR (400 MHz, CDCl₃) δ 8.21 (s, 1H), 7.39-7.30 (m, 2H), 6.99 (d, J=8.1 Hz, 1H), 6.92-6.83 (m, 2H), 6.78 (s, 1H), 5.85-5.74 (m, 1H), 2.32-2.10 (m, 9H), 1.78 (d, J=6.6 Hz, 3H). ¹⁹F NMR (376 MHz, CDCl₃) δ −57.82 (s). ³¹P NMR (162 MHz, CDCl₃) δ 30.42 (s).

Analytical method: Column: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

SFC Method: Instrument: Waters Thar 80 preparative SFC, Column: ChiralPak C-IG, 250×30 mm I.D., 5 μm, Mobile phase: A for CO₂ and B for 0.1% 7 mol/L NH₃ in MeOH, Gradient: B 30%, Flow rate: 40 mL/min, Back pressure: 100 bar, Column temperature: 35° C., Wavelength: 220 nm, Cycle-time: 9 min, Eluted time: 3 H.

### Illustration 51. Synthesis of (S)-1-(5-(3-(bis(methyl-d3) carbamoyl)phenoxy)-6-nitrobenzofuran-2-yl)ethyldi(aziridin-1-yl)phosphinate (533)

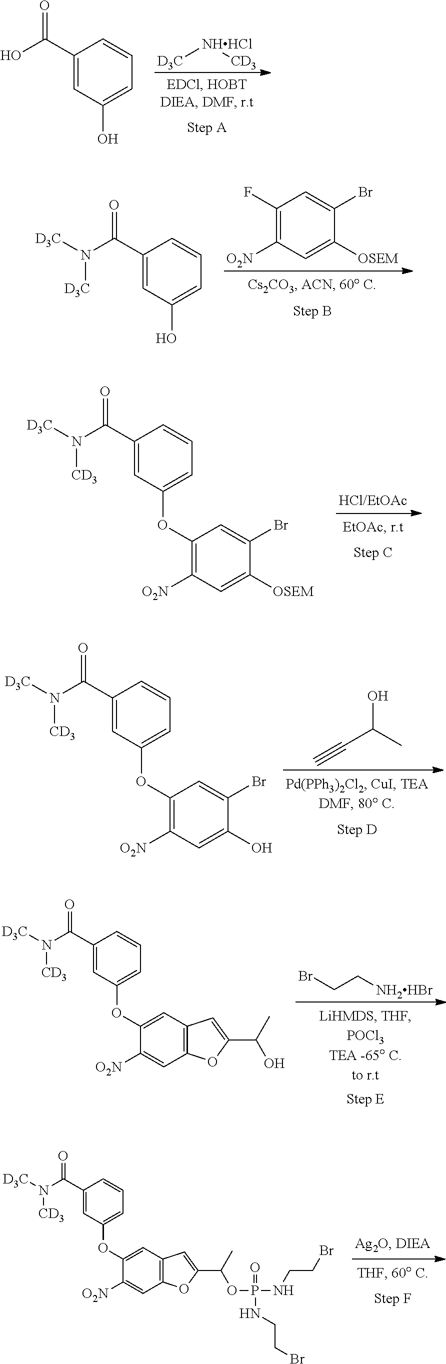

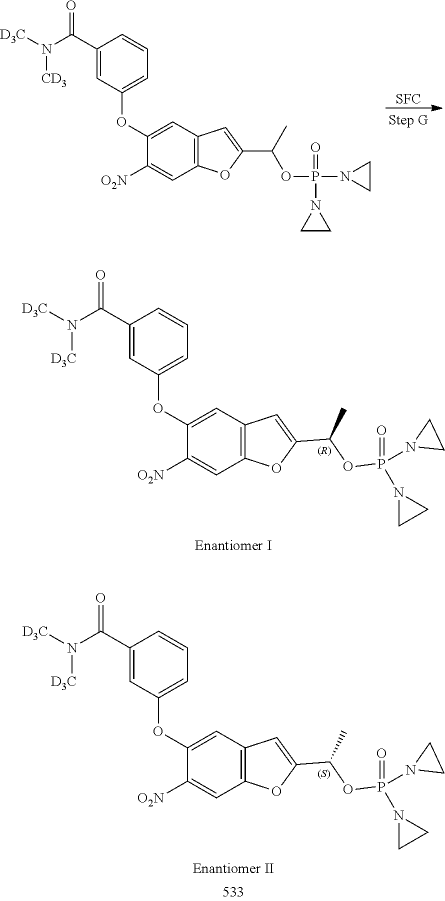

### STEP A: 3-hydroxy-N,N-bis(methyl-d3)benzamide

To a solution of 3-hydroxybenzoic acid (0.81 L, 8.69 mmol, 1.0 eq.), EDCI (2.17 g, 11.3 mmol, 1.3 eq.) and HOBt (1.41 g, 10.4 mmol, 1.2 eq.) in DMF (25 mL) were added DIEA (3.36 g, 26.1 mmol, 3.0 eq.) and bis(methyl-d3)amine hydrochloride (0.76 g, 8.69 mmol, 1.0 eq.). The resulting mixture was stirred at room temperature for 4 hrs. After completion, the reaction mixture was diluted with H₂O (20 mL), extracted with DCM (30 mL×3). The organic layers were combined and washed with brine (20 mL), dried over anhydrous Na₂SO₄, and concentrated under reduced pressure. The residue was purified by flash column chromatography on silica gel to afford 3-hydroxy-N, N-bis(methyl-d3)benzamide (0.56 g, 3.27 mmol, 38%) as a white solid. LCMS (ESI): m/z 172 [M+H]⁺.

### STEP B: 3-(5-bromo-2-nitro-4-((2-(trimethylsilyl) ethoxy)methoxy) phenoxy)-N,N-bis(methyl-d3)benzamide

To a solution of (2-((2-bromo-4-fluoro-5-nitrophenoxy)methoxy)ethyl)trimethylsilane (1 g, 2.73 mmol, 1.0 eq.) and 3-hydroxy-N,N-bis(methyl-d3)benzamide (0.56 g, 3.28 mmol, 1.2 eq.) in ACN (15 mL) was added Cs₂CO₃ (1.78 g, 5.46 mmol, 2.0 eq.). The resulting mixture was stirred at 60° C. for 0.5 hrs. After completion, the reaction mixture was concentrated under reduced pressure, the residue was purified by flash column chromatography on silica gel to afford 3-(5-bromo-2-nitro-4-((2-(trimethylsilyl)ethoxy)methoxy)phenoxy)-N,N-bis(methyl-d3)benzamide (1.2 g, 2.32 mmol, 85%) as a yellow oil. LCMS (ESI): m/z 517 [M+H]⁺.

### STEP C: 3-(5-bromo-4-hydroxy-2-nitrophenoxy)-N,N-bis(methyl-d3)benzamide

To a solution of 3-(5-bromo-2-nitro-4-((2-(trimethylsilyl)ethoxy)methoxy)phenoxy)-N,N-bis(methyl-d3)benzamide (1.2 g, 2.32 mmol, 1.0 eq.) in EtOAc (5 mL) was added HCl/EtOAc (5 mL). The mixture was stirred at room temperature for 2 hrs. After completion, the reaction mixture was concentrated under reduced pressure to give 3-(5-bromo-4-hydroxy-2-nitrophenoxy)-N,N-bis(methyl-d3)benzamide (880 mg, quant.) as a white solid, which was used in next step directly without further purification. LCMS (ESI): m/z 387 [M+H]⁺.

### STEP D: 3-((2-(1-hydroxyethyl)-6-nitrobenzofuran-5-yl)oxy)-N,N-bis(methyl-d3)benzamide

To a stirred solution of 3-(5-bromo-4-hydroxy-2-nitrophenoxy)-N,N-bis(methyl-d3)benzamide (880 mg, 2.27 mmol, 1.0 eq.) and but-3-yn-2-ol (319 mg, 4.55 mmol, 2.0 eq.) in DMF (10 mL) were added Pd(PPh₃)₂Cl₂ (160 mg, 0.23 mmol, 0.1 eq.), CuI (43 mg, 0.23 mmol, 0.1 eq.) and TEA (690 mg, 6.82 mmol, 3.0 eq.) at room temperature. The resulting mixture was stirred at 80° C. for 2 hrs under nitrogen atmosphere. After completion, the reaction mixture was diluted with H₂O (20 mL) and extracted with EtOAc (20 mL×3). The combined organic layers were washed with brine (20 mL), dried over anhydrous Na₂SO₄, and concentrated under reduced pressure. The residue was purified by flash column chromatography on silica gel to afford 3-((2-(1-hydroxyethyl)-6-nitrobenzofuran-5-yl)oxy)-N,N-bis(methyl-d3)benzamide (380 mg, 1.01 mmol, 44%) as a brown oil. LCMS (ESI): m/z 377 [M+H]⁺.

### STEP E: 1-{5-[(3-{[bis(trideuteriomethyl)amino]carbonyl}phenyl)oxy]-6-nitro-1-benzofuran-2-yl}ethyl P,P-bis[(2-bromoethyl)amino]phosphinate

To a solution of 3-((2-(1-hydroxyethyl)-6-nitrobenzofuran-5-yl)oxy)-N,N-bis(methyl-d3)benzamide (350 mg, 0.93 mmol, 1.0 eq.) in THF (70 mL) was added LiHMDS (1.9 mL, 1.86 mmol, 2.0 eq., 1 M in THF) at −65° C. The resulting mixture was stirred at −65° C. for 30 min. POCl₃ (285 mg, 1.86 mmol, 2.0 eq.) was added into the above mixture and the resulting mixture was stirred at −65° C. for 10 min. 2-bromoethan-1-amine hydrobromide (1.14 g, 5.58 mmol, 6.0 eq.) and TEA (1.13 g, 11.2 mmol, 12.0 eq.) was added into the above mixture. The resulting mixture was stirred at −65° C. for additional 30 min, then warmed up to room temperature and stirred for 1 hr. After completion, the reaction mixture was quenched with NH₄Cl (10 mL, sat. aq.) and extracted with EtOAc (15 mL×3). The combined organic layers were washed with brine (20 mL), dried over anhydrous Na₂SO₄, and concentrated under reduced pressure. The residue was purified by flash column chromatography on silica gel to afford 1-{5-[(3-{[bis(trideuteriomethyl)amino]carbonyl}phenyl)oxy]-6-nitro-1-benzofuran-2-yl}ethyl P,P-bis[(2-bromoethyl)amino]phosphinate (230 mg, 0.34 mmol, 37%) as a yellow oil. LCMS (ESI): m/z 667 [M+H]⁺.

### STEP F: 1-(5-(3-(bis(methyl-d3)carbamoyl)phenoxy)-6-nitrobenzofuran-2-yl)ethyl di(aziridin-1-yl)phosphinate

To a solution of 1-{5-[(3-{[bis(trideuteriomethyl)amino]carbonyl}phenyl)oxy]-6-nitro-1-benzofuran-2-yl}ethyl P,P-bis[(2-bromoethyl)amino]phosphinate (230 mg, 0.34 mmol, 1.0 eq.) and DIEA (222 mg, 1.72 mmol, 5.0 eq.) in THF (5 mL) was added Ag₂O (398 mg, 1.72 mmol, 5.0 eq.). The resulting mixture was stirred at 60° C. overnight. After completion, the reaction mixture was cooled to room temperature and filtered through a short pad of Celite®. The filtrate was concentrated under reduced pressure, the residue was purified by flash column chromatography on silica gel to give 1-(5-(3-(bis(methyl-d3) carbamoyl)phenoxy)-6-nitrobenzofuran-2-yl)ethyl di(aziridin-1-yl)phosphinate (95 mg, 0.19 mmol, 55%) as a yellow solid. LCMS (ESI): m/z 507 [M+H]⁺.

### STEP G: (R)-1-(5-(3-(bis(methyl-d3) carbamoyl)phenoxy)-6-nitrobenzofuran-2-yl)ethyl di(aziridin-1-yl)phosphinate & (S)-1-(5-(3-(bis(methyl-d3) carbamoyl)phenoxy)-6-nitrobenzofuran-2-yl)ethyl di(aziridin-1-yl)phosphinate (533)

1-(5-(3-(bis(methyl-d3) carbamoyl)phenoxy)-6-nitrobenzofuran-2-yl)ethyl di(aziridin-1-yl)phosphinate (95 mg, 0.19 mmol) was further separated by Chiral SFC to give:

Enantiomer I, (R)-1-(5-(3-(bis(methyl-d3) carbamoyl)phenoxy)-6-nitrobenzofuran-2-yl)ethyl di(aziridin-1-yl)phosphinate (10.8 mg, 23%); Retention time: 5.797 min, >99% ee. LC-MS (ESI): m/z 507.2 [M+H]⁺; ¹H NMR (400 MHz, CDCl₃) δ 8.17 (s, 1H), 7.39 (t, J=7.9 Hz, 1H), 7.28 (s, 1H), 7.16 (d, J=7.6 Hz, 1H), 7.10-7.02 (m, 1H), 6.98 (s, 1H), 6.74 (s, 1H), 5.81-5.74 (m, 1H), 2.29-2.12 (m, 8H), 1.77 (d, J=6.6 Hz, 3H). ³¹P NMR (162 MHz, CDCl₃) δ 30.38 (s).

Enantiomer II, (S)-1-(5-(3-(bis(methyl-d3) carbamoyl)phenoxy)-6-nitrobenzofuran-2-yl)ethyl di(aziridin-1-yl)phosphinate (533) (12.7 mg, 27%); Retention time: 8.922 min, >99% ee. LC-MS (ESI): m/z 507.2 [M+H]⁺; ¹H NMR (400 MHz, CDCl₃) δ 8.17 (s, 1H), 7.39 (t, J=7.9 Hz, 1H), 7.28 (s, 1H), 7.16 (d, J=7.6 Hz, 1H), 7.06 (d, J=8.3 Hz, 1H), 6.98 (s, 1H), 6.74 (s, 1H), 5.81-5.74 (m, 1H), 2.29-2.12 (m, 8H), 1.77 (d, J=6.6 Hz, 3H). ³¹P NMR (162 MHz, CDCl₃) δ 30.38 (s).

Analytical method: Column: Chiralpak AY-3 150; Á4.6 mm I.D., 3 μm, Mobile phase: 40% of ethanol (0.05% DEA) in CO₂, Flow rate: 2.5 mL/min, Column temp.: 35° C., ABPR: 1500 psi.

SFC Method: Instrument: MG II preparative SFC (SFC-13), Column: ChiralPak AY, 250×30 mm I.D., 10 μm, Mobile phase: A for CO₂ and B for Ethanol (0.1% NH₃H₂O), Gradient: B 40%, Flow rate: 70 mL/min, Back pressure: 100 bar, Column temperature: 38° C., Wavelength: 220 nm, Cycle time: ˜19 min.

TABLE 15
Characterization of compounds (see Illustration 49-51)

| Com- | | |
|---|---|---|
| pound | | |
| No. | Structure | LCMS, ¹H NMR (ppm); Retention time |
| 534 | 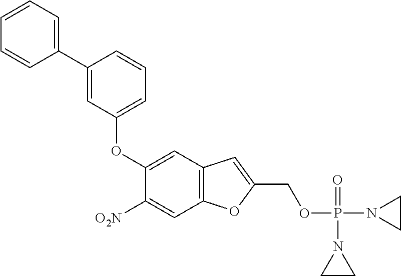 [SMILES: O=[N+]([O-])c1cc2oc(COP(=O)(N3CC3)N3CC3)cc2cc1Oc1cccc(-c2ccccc2)c1] | LCMS(ESI): m/z 482.10 [M + H]⁺ ¹H NMR (400 MHz, DMSO-d₆) δ 8.53 (d, J = 0.9 Hz, 1H), 7.68- 7.63 (m, 2H), 7.61 (s, 1H), 7.50- 7.43 (m, 4H), 7.40-7.35 (m, 1H), 7.34-7.32 (m, 1H), 7.13 (d, J = 0.9 Hz, 1H), 7.04-7.00 (m, 1H), 5.26 (d, J = 8.3 Hz, 2H), 2.15-2.07 (m, 8H). |
| 535 | 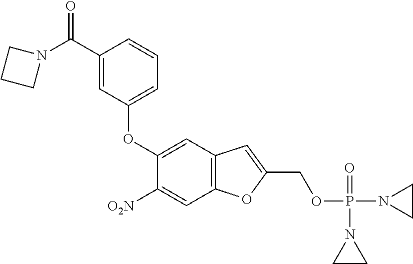 [SMILES: O=C(c1cccc(Oc2cc3cc(COP(=O)(N4CC4)N4CC4)oc3cc2[N+](=O)[O-])c1)N1CCC1] | LCMS(ESI): m/z 499.20 [M + H]⁺ ¹H NMR (400 MHz, DMSO-d₆) δ 8.55 (d, J = 0.9 Hz, 1H), 7.62 (s, 1H), 7.47 (t, J = 7.9 Hz, 1H), 7.41- 7.34 (m, 1H), 7.22-7.17 (m, 1H), 7.16-7.13 (m, 2H), 5.27 (d, J = 8.3 Hz, 2H), 4.24 (t, J = 7.6 Hz, 2H), 4.00 (t, J = 7.8 Hz, 2H), 2.22 (p, J = 7.7 Hz, 2H), 2.16- 2.07 (m, 8H). |
| 536 | 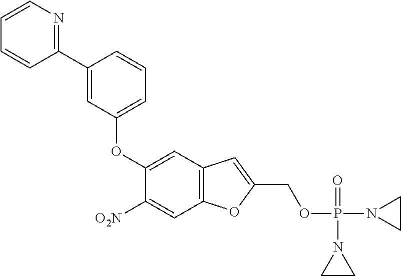 [SMILES: O=[N+]([O-])c1cc2oc(COP(=O)(N3CC3)N3CC3)cc2cc1Oc1cccc(-c2ccccn2)c1] | LCMS(ESI): m/z 493.20 [M + H]⁺ ¹H NMR (400 MHz, DMSO-d₆) δ 8.63 (d, J = 4.1 Hz, 1H), 8.55 (s, 1H), 7.98 (d, J = 8.0 Hz, 1H), 7.92-7.83 (m, 2H), 7.74 (t, J = 2.1 Hz, 1H), 7.62 (s, 1H), 7.52 (t, J = 8.0 Hz, 1H), 7.40-7.32 (m, 1H), 7.16-7.09 (m, 2H), 5.26 (d, J = 8.3 Hz, 2H), 2.17-2.07 (m, 8H). |
| 537 | 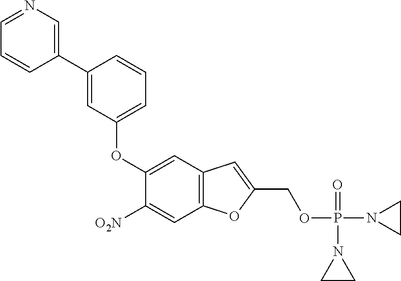 [SMILES: O=[N+]([O-])c1cc2oc(COP(=O)(N3CC3)N3CC3)cc2cc1Oc1cccc(-c2cccnc2)c1] | LCMS(ESI): m/z 493.10 [M + H]⁺ ¹H NMR (400 MHz, DMSO-d₆) δ 8.92-8.87 (m, 1H), 8.60-8.52 (m, 2H), 8.08 (d, J = 8.0 Hz, 1H), 7.61 (s, 1H), 7.57-7.43 (m, 4H), 7.13 (s, 1H), 7.07 (d, J = 7.6 Hz, 1H), 5.26 (d, J = 8.3 Hz, 2H), 2.18- 2.05 (m, 8H). |
| 538 | 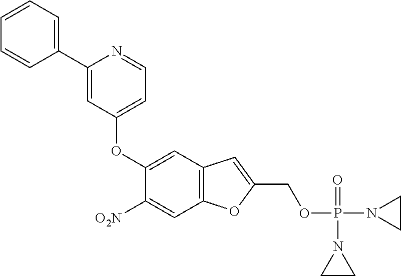 [SMILES: O=[N+]([O-])c1cc2oc(COP(=O)(N3CC3)N3CC3)cc2cc1Oc1ccnc(-c2ccccc2)c1] | LCMS(ESI): m/z 493.10 [M + H]⁺ ¹H NMR (400 MHz, DMSO-d₆) δ 8.64 (d, J = 0.9 Hz, 1H), 8.56 (d, J = 5.6 Hz, 1H), 8.08-8.02 (m, 2H), 7.86 (s, 1H), 7.57 (d, J = 2.4 Hz, 1H), 7.51-7.41 (m, 3H), 7.20 (s, 1H), 6.93 (dd, J = 5.6, 2.4 Hz, 1H), 5.30 (d, J = 8.2 Hz, 2H), 2.18-2.09 (m, 8H). |
| 539 | 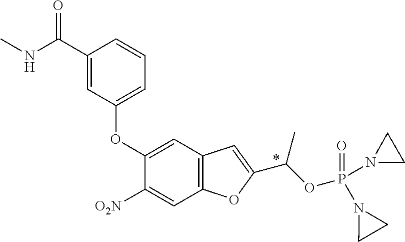 [SMILES: CNC(=O)c1cccc(Oc2cc3cc(C(C)OP(=O)(N4CC4)N4CC4)oc3cc2[N+](=O)[O-])c1] | LCMS(EST): m/z 487.10 [M + H]⁺ ¹H NMR (400 MHz, DMSO-d₆) δ 8.55 (d, J = 0.9 Hz, 1H), 8.49- 8.44 (m, 1H), 7.64-7.61 (m, 1H), 7.58 (s, 1H), 7.48 (t, J = 7.9 Hz, 1H), 7.43-7.41 (m, 1H), 7.22-7.18 (m, 1H), 7.08 (s, 1H), 5.78- 5.67 (m, 1H), 2.75 (d, J = 4.5 Hz, 3H), 2.17-2.02 (m, 8H), 1.67 (d, J = 6.6 Hz, 3H). ee: >99% Retention time: 1.433 min; Column: Cellulose SZ, 0.46 × 5 cm, 3.0 um; Mobile phase: Hex (0.1% DEA): EtOH = 50:50; Flow rate: 1.67 |
| | | mL/min; Temperature: ambient. |
| 540 | 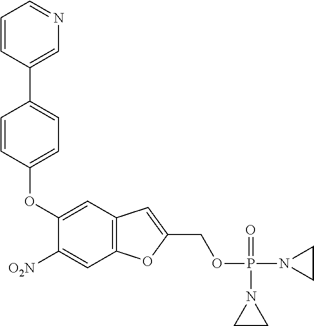 [SMILES: O=[N+]([O-])c1cc2oc(COP(=O)(N3CC3)N3CC3)cc2cc1Oc1ccc(-c2cccnc2)cc1] | LCMS(ESI): m/z 492.95 [M + H]⁺ ¹H NMR (400 MHz, Chloroform-d) δ 8.81 (d, J = 2.3 Hz, 1H), 8.59 (dd, J = 4.8, 1.6 Hz, 1H), 8.20 (s, 1H), 7.87-7.81 (m, 1H), 7.46 (t, J = 8.0 Hz, 1H), 7.38-7.32 (m, 3H), 7.23-7.20 (m, 1H), 7.04-7.00 (m, 1H), 6.82 (s, 1H), 5.27 (d, J = 8.3 Hz, 2H), 2.29-2.17 (m, 8H). |
| 541 | 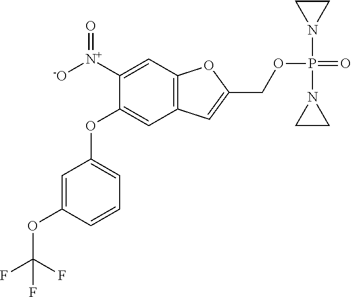 | LCMS(ESI): m/z 493.1 [M + H]⁺; ¹H NMR (400 MHz, CDCl₃) δ 8.47 (d, J = 2.8 Hz, 1H), 8.22 (s, 1H), 7.95 (d, J = 7.3 Hz, 2H), 7.73 (d, J = 8.7 Hz, 1H), 7.49-7.45 (m, 2H), 7.43-7.36 (m, 2H), 7.34 (s, 1H), 6.83 (s, 1H), 5.28 (d, J = 8.4 Hz, 2H), 2.29-2.16 (m, 8H). |
| 542 | 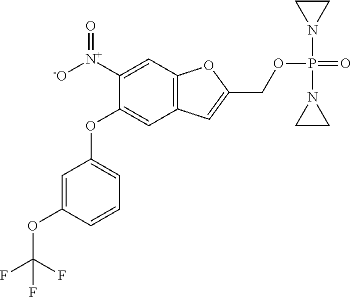 | LCMS(ESI): m/z 500.1 [M + H]⁺; ¹H NMR (400 MHz, CDCl₃) δ 8.21 (s, 1H), 7.38-7.32 (m, 2H), 7.01- 6.87 (m, 2H), 6.87-6.81 (m, 2H), 5.28 (d, J = 8.4 Hz, 2H), 2.28-2.18 (m, 8H). |
| 543 | 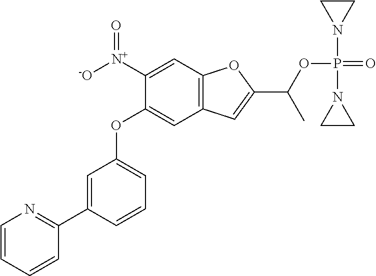 | LCMS(ESI): m/z 507.1 [M + H]⁺; ¹H NMR (400 MHz, CDCl₃) δ 8.66 (d, J = 4.5 Hz, 1H), 8.17 (s, 1H), 7.81-7.64 (m, 4H), 7.47 (t, J = 7.9 Hz, 1H), 7.26-7.21 (m, 2H), 7.07 (d, J = 7.7 Hz, 1H), 6.71 (s, 1H), 5.79-5.73 (m, 1H), 2.227-2.13 (m, 8H), 1.76 (d, J = 6.6 Hz, 3H). |
| 544 | 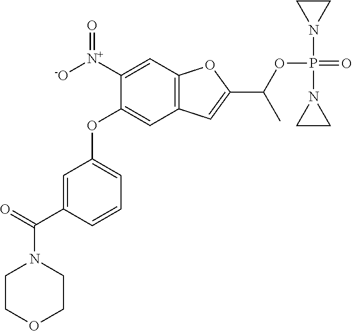 | LCMS(ESI): m/z 543.2 [M + H]⁺; ¹H NMR (400 MHz, CDCl₃) δ 8.18 (s, 1H), 7.40 (t, J = 7.9 Hz, 1H), 7.30 (s, 1H), 7.14 (d, J = 7.6 Hz, 1H), 7.07 (d, J = 8.4 Hz, 1H), 6.96 (s, 1H), 6.76 (s, 1H), 5.83-5.74 (m, 1H), 3.76-3.37 (m, 8H), 2.34-2.10 (m, 8H), 1.78 (d, J = 6.6 Hz, 3H). |
| 545 | 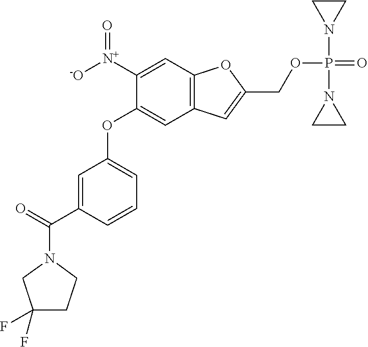 | LCMS(ESI): m/z 549.1 [M + H]⁺; ¹H NMR (400 MHz, CDCl₃) δ 8.19 (s, 1H), 7.42 (t, J = 7.6 Hz, 1H), 7.31 (s, 1H), 7.25-7.21 (m, 1H), 7.11 (d, J = 8.2 Hz, 1H), 7.06 (s, 1H), 6.83 (s, 1H), 5.27 (d, J = 8.4 Hz, 2H), 4.00-3.84 (m, 2H), 3.79- 3.65 (m, 2H), 2.45-2.33 (m, 2H), 2.30-2.18 (m, 8H). |
| 546 | 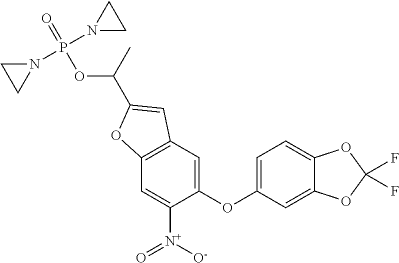 [SMILES: CC(OP(=O)(N1CC1)N1CC1)c1cc2cc(Oc3ccc4c(c3)OC(F)(F)O4)c([N+](=O)[O-])cc2o1] | LCMS(ESI): m/z 510.1 [M + H]⁺; ¹H NMR (400 MHz, CDCl₃) δ 8.17 (s, 1H), 7.23 (s, 1H), 7.01 (d, J = 8.7 Hz, 1H), 6.84-6.74 (m, 2H), 6.69 (d, J = 8.7 Hz, 1H), 5.84-5.72 (m, 1H), 2.39-2.06 (m, 8H), 1.77 (d, J = 6.6 Hz, 3H). |
| 547 | 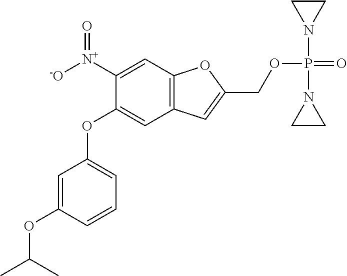 | LCMS(ESI): m/z 474.1 [M + H]⁺; ¹H NMR (400 MHz, CDCl₃) δ 8.15 (s, 1H), 7.22 (t, J = 8.1 Hz, 2H), 6.79 (s, 1H), 6.67 (d, J = 8.2 Hz, 1H), 6.60-6.51 (m, 2H), 5.25 (d, J = 8.4 Hz, 2H), 4.58-4.46 (m, 1H), 2.29-2.17 (m, 8H), 1.32 (d, J = 6.0 Hz, 6H). |
| 548 | 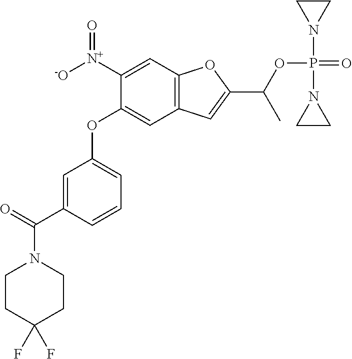 | LCMS(ESI): m/z 577.2 [M + H]⁺; ¹H NMR (400 MHz, CDCl₃) δ 8.18 (s, 1H), 7.41 (t, J = 7.5 Hz, 1H), 7.30 (s, 1H), 7.14 (d, J = 7.4 Hz, 1H), 7.08 (d, J = 8.5 Hz, 1H), 6.96 (s, 1H), 6.76 (s, 1H), 5.85-5.71 (m, 1H), 3.83-3.53 (m, 4H), 2.27- 2.15 (m, 8H), 2.09-1.90 (m, 4H), 1.78 (d, J = 6.6 Hz, 3H). |
| 549 | 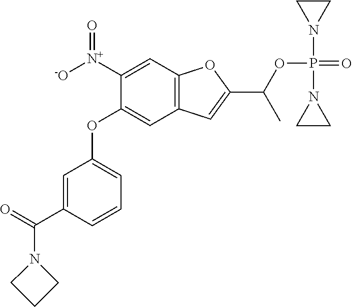 | LCMS(ESI): m/z 513.1 [M + H]⁺; ¹H NMR (400 MHz, CDCl₃) δ 8.18 (s, 1H), 7.45-7.30 (m, 2H), 7.18 (s, 1H), 7.12 (d, J = 7.9 Hz, 1H), 6.75 (s, 1H), 5.86-5.68 (m, 1H), 4.22 (m, 4H), 2.45-2.03 (m, 10H), 1.90-1.72 (m, 3H). |
| 550 | 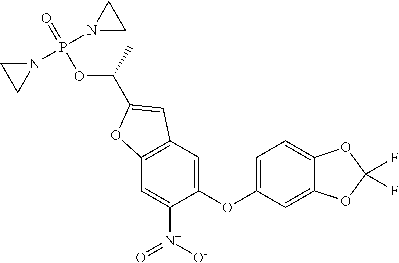 [SMILES: C[C@@H](OP(=O)(N1CC1)N1CC1)c1cc2cc(Oc3ccc4c(c3)OC(F)(F)O4)c([N+](=O)[O-])cc2o1] | LCMS(ESI): m/z 510.0 [M + H]⁺; ¹H NMR (400 MHz, CDCl₃) δ 8.17 (s, 1H), 7.23 (s, 1H), 7.01 (d, J = 8.6 Hz, 1H), 6.80 (s, 1H), 6.76 (s, 1H), 6.69 (d, J = 8.7 Hz, 1H), 5.85- 5.70 (m, 1H), 2.36-2.09 (m, 8H), 1.77 (d, J = 6.6 Hz, 3H). ee: >99% Retention time: 2.270 min (Method 1). |
| 551 | 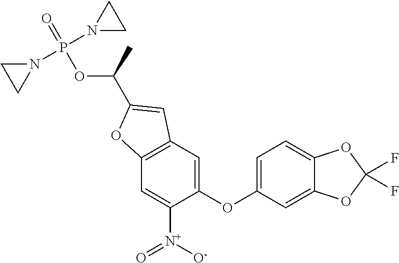 [SMILES: C[C@H](OP(=O)(N1CC1)N1CC1)c1cc2cc(Oc3ccc4c(c3)OC(F)(F)O4)c([N+](=O)[O-])cc2o1] | LCMS(ESI): m/z 510.0 [M + H]⁺; ¹H NMR (400 MHz, CDCl₃) δ 8.17 (s, 1H), 7.23 (s, 1H), 7.01 (d, J = 8.6 Hz, 1H), 6.80 (s, 1H), 6.76 (s, 1H), 6.69 (d, J = 8.5 Hz, 1H), 5.83- 5.72 (m, 1H), 2.28-2.12 (m, 9H), 1.77 (d, J = 6.5 Hz, 3H). ee: >99% Retention time: 2.608 min (Method 1). |
| 552 | 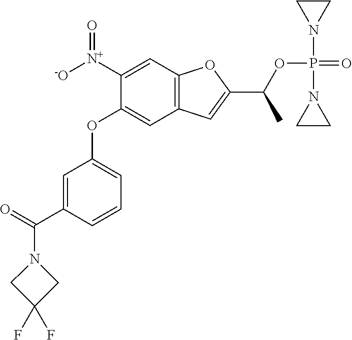 | LCMS(ESI): m/z 549.2 [M + H]⁺; ¹H NMR (400 MHz, CDCl₃) δ 8.21 (s, 1H), 7.42 (t, J = 7.8 Hz, 1H), 7.37-7.29 (m, 2H), 7.23-7.11 (m, 2H), 6.78 (s, 1H), 5.90-5.67 (m, 1H), 4.64 (m, 4H), 2.27-2.19 (m, 8H), 1.78 (d, J = 6.5 Hz, 3H). ee: >99% Retention time: 5.395 min (Method 1). |
| 553 | 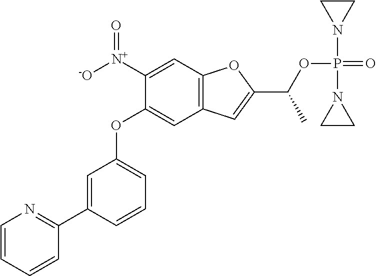 | LCMS(ESI): m/z 507.1 [M + H]⁺; ¹H NMR (400 MHz, CDCl₃) δ 8.66 (d, J = 3.8 Hz, 1H), 8.18 (s, 1H), 7.81-7.65 (m, 4H), 7.47 (t, J = 7.9 Hz, 1H), 7.6-7.23 (m, 2H), 7.07 (d, J = 8.6 Hz, 1H), 6.72 (s, 1H), 5.86- 5.68 (m, 1H), 2.35-2.08 (m, 8H), 1.77 (d, J = 6.6 Hz, 3H). ee: 97% Retention time: 3.708 min (Method 7). |
| 554 | 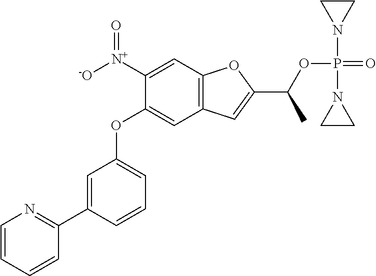 | LCMS(ESI): m/z 507.1 [M + H]⁺; ¹H NMR (400 MHz, CDCl₃) δ 8.66 (d, J = 3.8 Hz, 1H), 8.18 (s, 1H), 7.81-7.65 (m, 4H), 7.47 (t, J = 7.9 Hz, 1H), 7.6-7.23 (M, 2H), 7.07 (d, J = 8.6 Hz, 1H), 6.72 (s, 1H), 5.86- 5.68 (m, 1H), 2.35-2.08 (m, 8H), 1.77 (d, J = 6.6 Hz, 3H). ee: 97% Retention time: 5.249 min (Method 7). |
| 555 | 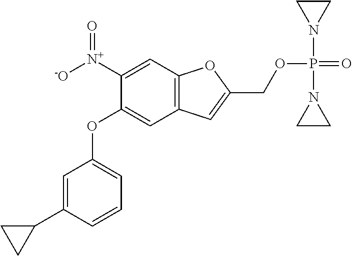 | LCMS(ESI): m/z 456.1 [M + H]⁺; ¹H NMR (400 MHz, CDCl₃) δ 8.14 (s, 1H), 7.25-7.19 (m, 2H), 6.86 (d, J = 7.6 Hz, 1H), 6.76 (d, J = 8.0 Hz, 3H), 5.25 (d, J = 8.4 Hz, 2H), 2.30-2.16 (m, 8H), 1.86 (d, J = 4.8 Hz, 1H), 0.97 (d, J = 8.0 Hz, 2H), 0.69 (d, J = 4.7 Hz, 2H). |
| 556 | 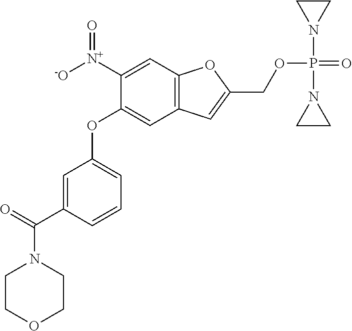 | LCMS(ESI): m/z 529.2 [M + H]⁺; ¹H NMR (400 MHz, CDCl₃) δ 8.17 (s, 1H), 7.39 (t, J = 7.9 Hz, 1H), 7.30 (s, 1H), 7.14 (d, J = 7.5 Hz, 1H), 7.06 (d, J = 8.3 Hz, 1H), 6.96 (s, 1H), 6.82 (s, 1H), 5.26 (d, J = 8.3 Hz, 2H), 3.80-3.43 (m, 8H), 2.29-2.18 (m, 8H). |
| 557 | 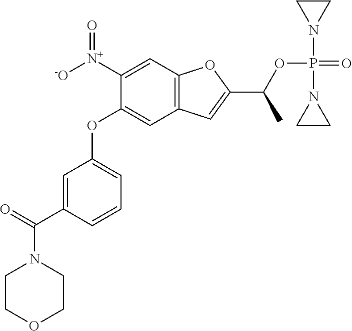 | LCMS(ESI): m/z 543.1 [M + H] ; ¹H NMR (400 MHz, CDCl₃) δ 8.16 (s, 1H), 7.38 (t, J = 7.8 Hz, 1H), 7.28 (s, 1H), 7.13 (d, J = 7.4 Hz, 1H), 7.05 (d, J = 8.1 Hz, 1H), 6.95 (s, 1H), 6.74 (s, 1H), 5.83-5.71 (m, 1H), 3.80-3.36 (m, 8H), 2.30-2.10 (m, 8H), 1.76 (d, J = 6.5 Hz, 3H). ee: 94% Retention time: 2.426 min (Method 50). |
| 558 | 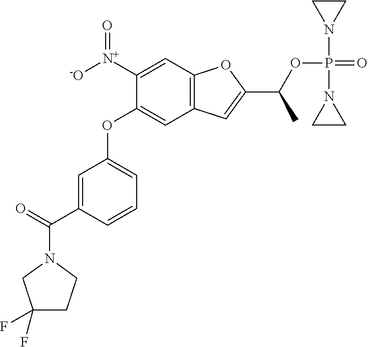 | LCMS(ESI): m/z 563.2 [M + H]⁺; ¹H NMR (400 MHz, CDCl₃) δ 8.19 (s, 1H), 7.41 (t, J = 7.9 Hz, 1H), 7.30 (s, 1H), 7.23 (s, 1H), 7.11 (d, J = 8.4 Hz, 1H), 7.06 (s, 1H), 6.77 (s, 1H), 5.82-5.73 (m, 1H), 4.02-3.61 (m, 4H), 2.39 (s, 2H), 2.30-2.12 (m, 8H), 1.78 (d, J = 6.5 Hz, 3H). ee: >99% Retention time: 5.231 min (Method 1). |
| 559 | 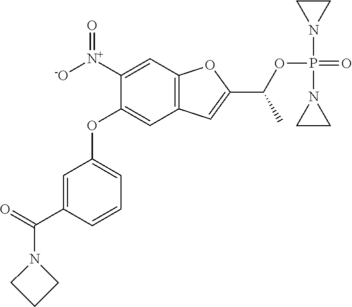 | LCMS(ESI): m/z 513.1 [M + H]⁺; ¹H NMR (400 MHz, CDCl₃) δ 8.18 (s, 1H), 7.42-7.32 (m, 2H), 7.27 (s,1H), 7.18 (s, 1H), 7.12 (d, J = 7.8 Hz, 1H), 6.75 (s, 1H), 5.84-5.71 (m, 1H), 4.28-4.17( 4H), 2..32-2.23 (m, 10H), 1.78 (d, J = 6.4 Hz, 3H). ee: >99% Retention time: 6.871 min (Method 3). |
| 560 | 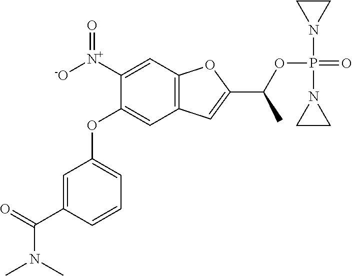 | LCMS(ESI): m/z 502.1 [M + H]⁺; ¹H NMR (400 MHz, DMSO) δ 8.58 (s, 1H), 8.48 (s, 1H), 8.39 (s, 1H), 7.73 (s, 1H), 7.46 (s, 1H), 7.10 (s, 1H), 5.79-5.68 (m, 1H), 2.95 (s, 3H), 2.88 (s, 3H), 2.20-2.01 (m, 8H), 1.68 (d, J = 5.5 Hz, 3H). ee: 97% Retention time: 3.281 min (Method 26) |
| 561 | 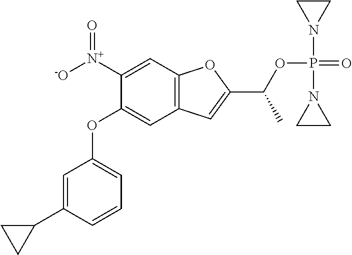 | LCMS(ESI): m/z 470 [M + H]⁺; ¹H NMR (400 MHz, CDCl₃) δ 8.14 (s, 1H), 7.24-7.14 (m, 2H), 6.85 (d, J = 7.8 Hz, 1H), 6.80-6.67 (m, 3H), 5.84-5.72 (m, 1H), 2.29-2.11 (m, 8H), 1.90-1.82 (m, 1H), 1.76 (d, J = 6.0 Hz, 3H), 1.01-0.89 (m, 2H), 0.72-0.62 (m, 2H). ee: 99% Retention time: 4.580 min (Method 1). |
| 562 | 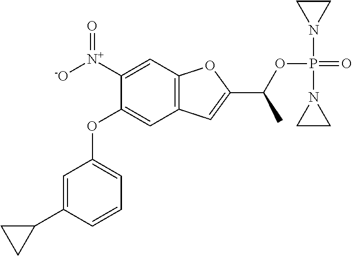 | LCMS(ESI): m/z 470 [M + H]⁺; ¹H NMR (400 MHz, CDCl₃) δ 8.14 (s, 1H), 7.24-7.14 (m, 2H), 6.85 (d, J = 7.3 Hz, 1H), 6.79-6.70 (m, 3H), 5.81-5.68 (m, 1H), 2.28-2.10 (m, 8H), 1.94-1.82 (m, 1H), 1.76 (d, J = 6.3 Hz, 3H), 0.97 (d, J = 7.7 Hz, 2H), 0.73-0.61 (m, 2H). ee: 96% Retention time: 5.615 min (Method 1). |
| 563 | 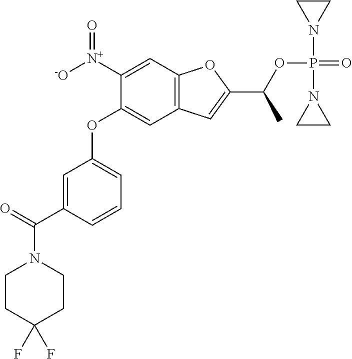 | LCMS(ESI): m/z 577.1 [M + H]⁺; ¹H NMR (400 MHz, CDCl₃) δ 8.18 (s, 1H), 7.41 (t, J = 7.3 Hz, 1H), 7.31 (s, 1H), 7.14 (d, J = 7.4 Hz, 1H), 7.08 (d, J = 7.5 Hz, 1H), 6.96 (s, 1H), 6.76 (s, 1H), 5.84-5.70 (m, 1H), 3.83-3.54 (m, 4H), 2.37-2.09 (m, 8H), 2.04-1.88 (m, 4H), 1.78 (d, J = 6.3 Hz, 3H). ee: 98% Retention time: 4.650 min (Method 30). |
| 564 | 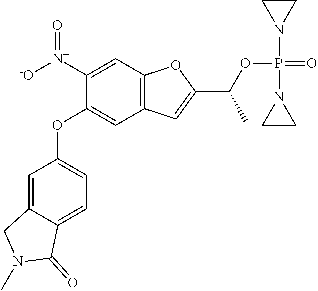 | LCMS(ESI): m/z 499.2 [M + H] * ; ¹H NMR (400 MHz, CDCl₃) δ 8.22 (s, 1H), 7.79 (d, J = 8.2 Hz, 1H), 7.33 (s, 1H), 7.05-6.99 (m, 2H), 6.78 (s, 1H), 5.82-5.71 (m, 1H), 4.33 (s, 2H), 3.18 (s, 3H), 2.29-2.03 (m, 8H), 1.82-1.74 (m, 3H). ee: >99% Retention time: 3.328 min (Method 50). |
| 565 | 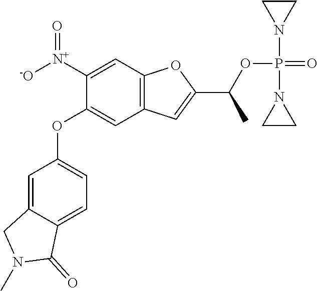 | LCMS(ESI): m/z 499.2 [M + H]⁺; ¹H NMR (400 MHz, CDCl₃) δ 8.22 (s, 1H), 7.79 (d, J = 8.2 Hz, 1H), 7.33 (s, 1H), 7.05-6.99 (m, 2H), 6.78 (s, 1H), 5.82-5.71 (m, 1H), 4.33 (s, 2H), 3.18 (s, 3H), 2.29-2.03 (m, 8H), 1.82-1.74 (m, 3H). ee: 80% Retention time: 3.862 min (Method 50). |
| 566 | 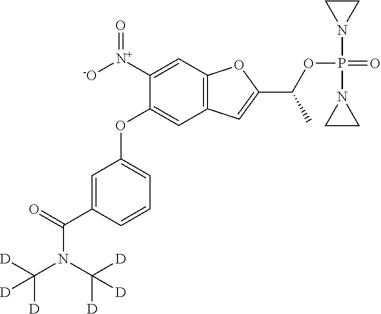 | LCMS(ESI): m/z 507.2 [M + H]⁺; ¹H NMR (400 MHz, CDCl₃) δ 8.17 (s, 1H), 7.39 (t, J = 7.9 Hz, 1H), 7.28 (s, 1H), 7.16 (d, J = 7.6 Hz, 1H), 7.10-7.02 (m, 1H), 6.98 (s, 1H), 6.74 (s, 1H), 5.81-5.74 (m, 1H), 2.29-2.12 (m, 8H), 1.77 (d, J = 6.6 Hz, 3H). ee: >99% Retention time: 5.797 min (Method 25). |
| 567 | 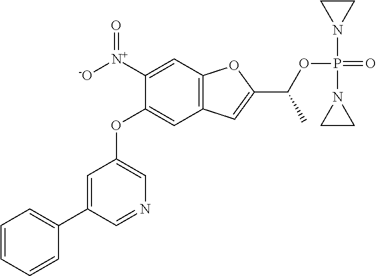 | LCMS(ESI): m/z 507.1 [M + H]⁺; ¹H NMR (400 MHz, CDCl₃) δ 8.64 (s, 1H), 8.35 (s, 1H), 8.23 (s, 1H), 7.57-7.53 (m, 2H), 7.49-7.47 (m, 2H), 7.46-7.39 (m, 2H), 7.35 (s, 1H), 6.78 (s, 1H), 5.90-5.68 (m, 1H), 2.30-2.12 (m, 8H), 1.78 (d, J = 6.6 Hz, 3H). ee: >99% Retention time: 3.762 min (Method 26). |
| 568 | 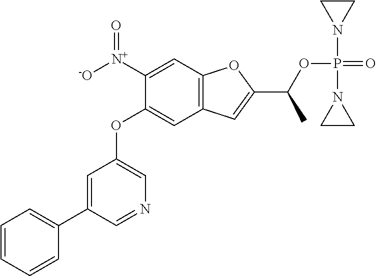 | LCMS(ESI): m/z 507.1 [M + H]⁺; ¹H NMR (400 MHz, CDCl₃) δ 8.63 (s, 1H), 8.35 (d, J = 2.6 Hz, 1H), 8.23 (s, 1H), 7.57-7.53 (m, 2H), 7.49-7.46 (m, 2H), 7.46-7.38 (m, 2H), 7.35 (d, J = 2.4 Hz, 1H), 6.78 (s, 1H), 5.87-5.66 (m, 1H), 2.30- 2.11 (m, 8H), 1.78 (d, J = 6.6 Hz, 3H). ee: >99% Retention time: 4.254 min (Method 26). |
| 569 | 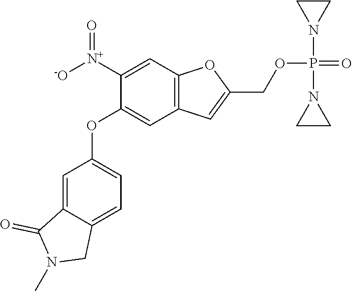 | LCMS(ESI): m/z 485.1 [M + H]⁺; ¹H NMR (400 MHz, CDCl₃) δ 8.21 (s, 1H), 7.44 (d, J = 8.2 Hz, 1H), 7.34-7.27 (m, 2H), 7.24 (d, J = 2.3 Hz, 1H), 6.82 (s, 1H), 5.28 (d, J = 8.4 Hz, 2H), 4.36 (s, 2H), 3.19 (s, 3H), 2.32-2.13 (m, 8H). |

### Chiral Separation Methods and Conditions

Method 1: Column: ChiralPak C-IG, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

Method 2: Column: ChiralPak IH, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

Method 3: Column: ChiralPak C-IG, 100×4.6 mm I.D., 5 μm; Mobile phase: A for CO₂ and B for methanol (0.05% DEA); Gradient: 8 min @ 50% B; Flow rate: 2.5 mL/min; Column temperature: 40° C.

Method 4: Column: ChiralCel OJ, 250×4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C.

Method 5: Column: ChiralPak AD, 250×4.6 mm I.D., 5 μm, Mobile phase: A for CO₂ and B for methanol (0.05% DEA), Gradient: 8 min @B 40%, Flow rate: 2.0 mL/min, Back pressure: 100 bar, Column temperature: 35° C.

Method 6: Column: ChiralPak IB, 100×4.6 mm I.D., 5 μm; Mobile phase: A for CO₂ and B for methanol (0.05% DEA); Gradient: 0.0 min-1.0 min @ 10% B, 1.0 min-4.5 min gradient (10-40% B), 4.5 min-7.0 min @ 40% B, 7.0 min-8.0 min @ 10% B; Flow rate: 2.5 mL/min; Column temperature: 40° C.

Method 7: Column: ChiralPak IB, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

Method 8: Column: ChiralCel OZ, 100×4.6 mm I.D., 5 μm; Mobile phase: A for CO₂ and B for Isopropyl alcohol (0.05% DEA); Gradient: 8 min @ 40% B; Flow rate: 2.5 mL/min; Column temperature: 40° C.

Method 9: Column: ChiralPak IB, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

Method 10: Column: ChiralCel OD, 250×4.6 mm I.D., 5 μm, Mobile phase: A for CO₂ and B for MeOH (0.05% DEA), Gradient: 8 min @ B 30%, Flow rate: 2.0 mL/min, Back pressure: 100 bar, Column temperature: 35° C.

Method 11: Column: ChiralPak AD, 250×4.6 mm I.D., 5 μm, Mobile phase: A for CO₂ and B for methanol (0.05% DEA), Gradient: 8 min @B 50%, Flow rate: 1.8 mL/min, Back pressure: 100 bar, Column temperature: 35° C.

Method 12: Column: ChiralCel OJ, 250×4.6 mm I.D., 5 μm, Mobile phase: A for CO₂ and B for MeOH (0.05% DEA), Gradient: 8 min @ B 40%, Flow rate: 2.0 mL/min, Back pressure: 100 bar, Column temperature: 35° C.

Method 13: Column: ChiralPak IB, 100×4.6 mm I.D., 5 μm; Mobile phase: A for CO₂ and B for ethanol (0.05% DEA); Gradient: 8 min @ 20% B; Flow rate: 2.5 mL/min; Column temperature: 40° C.

Method 14: Column: ChiralPak IB, 100×4.6 mm I.D., 5 μm; Mobile phase: A for CO₂ and B for Isopropyl alcohol (0.05% DEA); Gradient: 8 min @ 20% B; Flow rate: 2.5 mL/min; Column temperature: 40° C.

Method 15: Column: ChiralPak AD, 250×4.6 mm I.D., 5 μm, Mobile phase: A for CO₂ and B for ethanol (0.05% DEA), Gradient: 8 min @B 40%, Flow rate: 2.0 mL/min, Back pressure: 100 bar, Column temperature: 35° C.

Method 16: Column: ChiralPak IA, 100×4.6 mm I.D., 5 μm; Mobile phase: A for CO₂ and B for ethanol (0.05% DEA); Gradient: 8 min @ 10% B; Flow rate: 2.5 mL/min; Column temperature: 40° C.

Method 17: Column: ChiralCel OZ, 100×4.6 mm I.D., 5 μm; Mobile phase: A for CO₂ and B for Isopropyl alcohol (0.05% DEA); Gradient: 8 min @ 20% B; Flow rate: 2.5 mL/min; Column temperature: 40° C.

Method 18: Column: ChiralCel OZ, 100×4.6 mm I.D., 5 μm; Mobile phase: A for CO₂ and B for ethanol (0.05% DEA); Gradient: 8 min @ 30% B; Flow rate: 2.5 mL/min; Column temperature: 40° C.

Method 19: Column: ChiralPak AS, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

Method 20: Column: ChiralPak AY, 100×4.6 mm I.D., 5 μm; Mobile phase: A for CO₂ and B for Isopropyl alcohol (0.05% DEA); Gradient: 8 min @ 20% B; Flow rate: 2.5 mL/min; Column temperature: 40° C.

Method 21: Column: ChiralPak C-IG, 100×4.6 mm I.D., 5 μm; Mobile phase: A for CO₂ and B for ethanol (0.05% DEA); Gradient: 8 min @ 30% B; Flow rate: 2.5 mL/min; Column temperature: 40° C.

Method 22: Column: ChiralPak C-IG, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

Method 23: Column: ChiralPak C-IG, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

Method 24: Column: ChiralCel OZ, 100×4.6 mm I.D., 5 μm; Mobile phase: A for CO₂ and B for methanol (0.05% DEA); Gradient: 8 min @ 40% B; Flow rate: 2.5 mL/min; Column temperature: 40° C.

Method 25: Column: Chiralpak AY-3 150; Á4.6 mm I.D., 3 μm, Mobile phase: 40% of ethanol (0.05% DEA) in CO₂, Flow rate: 2.5 mL/min, Column temp.: 35° C., ABPR: 1500 psi.

Method 26: Column: ChiralPak IB, 100×4.6 mm I.D., 5 μm; Mobile phase: A for CO₂ and B for ethanol (0.05% DEA); Gradient: 8 min @ 25% B; Flow rate: 2.5 mL/min; Column temperature: 40° C.

Method 27: Column: ChiralPak IC, 100;Á4.6 mm I.D., 3 μm, Mobile phase: A for CO₂ and B for methanol (0.05% DEA), Gradient: 8 min @B 40%, Flow rate: 2.0 mL/min, Back pressure: 100 bar, Column temperature: 35° C.

Method 28: Column: ChiralCel OJ, 100;Á4.6 mm I.D., 3 μm, Mobile phase: A for CO₂ and B for methanol (0.05% DEA), Gradient: 8 min @B 20%, Flow rate: 2.5 mL/min, Back pressure: 100 bar, Column temperature: 35° C.

Method 29: Column: Column: ChiralPak AD, 100;Á4.6 mm I.D., 3 μm, Mobile phase: A for CO₂ and B for ethanol (0.05% DEA), Gradient: 8 min @B 30%, Flow rate: 2.5 mL/min, Back pressure: 100 bar, Column temperature: 35° C.

Method 30: Column: ChiralPak IG, 100;Á4.6 mm I.D., 5 μm, Mobile phase: A for CO₂ and B for methanol (0.05% DEA), Gradient: 8 min @B 40%, Flow rate: 2.5 mL/min, Back pressure: 100 bar, Column temperature: 35° C.

Method 31: Column: ChiralPak IH, 100×4.6 mm I.D., 5 μm; Mobile phase: A for CO₂ and B for ethanol (0.05% DEA); Gradient: 8 min @ 20% B; Flow rate: 2.5 mL/min; Column temperature: 40° C.

Method 32: Column: ChiralPak AD, 100;Á4.6 mm I.D., 3 μm, Mobile phase: A for CO₂ and B for methanol (0.05% DEA), Gradient: 8 min @B 30%, Flow rate: 2.5 mL/min, Back pressure: 100 bar, Column temperature: 35° C.

Method 33: Column: ChiralPak AD, 100;Á4.6 mm I.D., 3 μm, Mobile phase: A for CO₂ and B for isopropanol (0.05% DEA), Gradient: 8 min @B 30%, Flow rate: 2.5 mL/min, Back pressure: 100 bar, Column temperature: 35° C.

Method 34: Column: ChiralCel OZ, 100×4.6 mm I.D., 5 μm; Mobile phase: A for CO₂ and B for methanol (0.05% DEA); Gradient: 8 min @ 50% B; Flow rate: 1.8 mL/min; Column temperature: 40° C.

Method 35: Column: ChiralCel OX, 100×4.6 mm I.D., 5 μm; Mobile phase: A for CO₂ and B for Isopropyl alcohol (0.05% DEA); Gradient: 8 min @ 40% B; Flow rate: 2.5 mL/min; Column temperature: 40° C.

Method 36: Column: ChiralPak AD, 100; Á4.6 mm I.D., 3 μm, Mobile phase: A for CO₂ and B for ethanol (0.05% DEA), Gradient: 8 min @B 20%, Flow rate: 2.5 mL/min, Back pressure: 100 bar, Column temperature: 35° C.

Method 37: Column: ChiralPak IH, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

Method 38: Column: Column: Chiralpak AD-3 50, Á4.6 mm I.D., 3 μm Mobile phase: A: CO₂ B: ethanol (0.05% DEA) Gradient: from 5% to 40% of B in 4 min and from 40% to 5% of B in 0.2 min, then hold 5% of B for 1.8 minFlow rate: 3 mL/minColumn temp.: 35° C. ABPR: 1500 psi.

Method 39: Column: Column: ChiralPak AD, 100;Á4.6 mm I.D., 3 μm, Mobile phase: A for CO₂ and B for EtOH (0.05% DEA), Gradient: 1 min @B 5%, 4 min gradient (5-40%), 2 min @B 40%, then 1.5 min @B 5%, Flow rate: 2.5 mL/min, Back pressure: 100 bar, Column temperature: 35° C.

Method 40: Column: ChiralCel OX, 100; Á4.6 mm I.D., 5 μm; Mobile phase: A for CO₂ and B for ethanol (0.05% DEA); Gradient: 8 min @ 20% B; Flow rate: 2.5 mL/min; Column temperature: 40° C.

Method 41: Column: ChiralCel OD, 100; Á4.6 mm I.D., 3 μm, Mobile phase: A for CO₂ and B for EtOH (0.05% DEA), Gradient: 1 min @B 5%, 4 min gradient (5-40%), 2 min @B 40%, then 1.5 min @B 5%, Flow rate: 2.5 mL/min, Back pressure: 100 bar, Column temperature: 35° C.

Method 42: Column: ChiralCel OD, 100;Á4.6 mm I.D., 3 μm, Mobile phase: A for CO₂ and B for methanol (0.05% DEA), Gradient: 8 min @B 40%, Flow rate: 2.0 mL/min, Back pressure: 100 bar, Column temperature: 35° C.

Method 43: Column: ChiralPak AD, 100; Á4.6 mm I.D., 3 μm, Mobile phase: A for CO₂ and B for isopropanol (0.05% DEA), Gradient: 8 min @B 40%, Flow rate: 2.0 mL/min, Back pressure: 100 bar, Column temperature: 35° C.

Method 44: Column: ChiralPak IA, 100;Á4.6 mm I.D., 3 μm, Mobile phase: A for CO₂ and B for methanol (0.05% DEA), Gradient: 8 min @B 40%, Flow rate: 2.0 mL/min, Back pressure: 100 bar, Column temperature: 35° C.

Method 45: Column: ChiralCel OZ, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

Method 46: Column: ChiralPak AY, 100×4.6 mm I.D., 5 μm; Mobile phase: A for CO₂ and B for methanol (0.05% DEA); Gradient: 8 min @ 30% B; Flow rate: 2.5 mL/min; Column temperature: 40° C.

Method 47: Column: ChiralPak C-IC, 100×4.6 mm I.D., 5 μm; Mobile phase: A for CO₂ and B for methanol (0.05% DEA); Gradient: 8 min @ 40% B; Flow rate: 2.0 mL/min; Column temperature: 40° C.

Method 48: Column: ChiralPak C-IG, 100×4.6 mm I.D., 5 μm; Mobile phase: A for CO₂ and B for ethanol (0.05% DEA); Gradient: 8 min @ 20% B; Flow rate: 2.5 mL/min; Column temperature: 40° C.

Method 49: Column: Column: ChiralPak AD, 100;Á4.6 mm I.D., 3 μm, Mobile phase: A for CO₂ and B for methanol (0.05% DEA), Gradient: 8 min @B 20%, Flow rate: 2.5 mL/min, Back pressure: 100 bar, Column temperature: 35° C.

Method 50: Column: ChiralPak IB, 100×4.6 mm I.D., 5 μm; Mobile phase: A for CO₂ and B for ethanol (0.05% DEA); Gradient: 8 min @ 30% B; Flow rate: 2.5 mL/min; Column temperature: 40° C.

Method 51: Column: Column: (R,R)-WHELK, 100; Á4.6 mm I.D., 3.5 μm, Mobile phase: A for CO₂ and B for methanol (0.05% DEA), Gradient: 8 min @B 40%, Flow rate: 2.0 mL/min, Back pressure: 100 bar, Column temperature: 35° C.

Method 52: Column: ChiralPak AS, 100×4.6 mm I.D., 5 μm; Mobile phase: A for CO₂ and B for methanol (0.05% DEA); Gradient: 8 min @ 20% B; Flow rate: 2.5 mL/min; Column temperature: 40° C.

Method 53: Column: ChiralPak C-IG, 100×4.6 mm I.D., 5 μm; Mobile phase: A for CO₂ and B for methanol; Gradient: 8 min @ 10% B; Flow rate: 2.5 mL/min; Column temperature: 40° C.

Method 54: Column: ChiralPak AS, 100×4.6 mm I.D., 5 μm; Mobile phase: A for CO₂ and B for methanol; Gradient: 8 min @ 20% B; Flow rate: 2.5 mL/min; Column temperature: 40° C.

Method 55: Column: ChiralPak IG, 100;Á4.6 mm I.D., 5 μm, Mobile phase: A for CO₂ and B for methanol (0.05% DEA), Gradient: 8 min @B 30%, Flow rate: 2.5 mL/min, Back pressure: 100 bar, Column temperature: 35° C.

Method 56: Column: ChiralPak C-IC, 100×4.6 mm I.D., 5 μm; Mobile phase: A for CO₂ and B for methanol (0.05% DEA); Gradient: 8 min @ 30% B; Flow rate: 2.0 mL/min; Column temperature: 40° C.

### Biological Example 1. In Vitro Human Cancer Cell Line Cytotoxicity Assay

IC₅₀ values in nanomolar reported here were derived from in vitro proliferation assay data in non-small cell lung cancer cell line H460.

Specifically, exponentially growing cells were seeded at density of 3×10³ cells per well in 96-well plate and incubated at 37° C. in 5% CO₂, 95% air and 100% relative humidity for 24 hours before adding compounds. Compounds were solubilized in 100% DMSO at 10 mM. Compounds were added to the plate using Tecan D300e® digital Dispenser. After drug addition, the plates were incubated for an additional 72 hours at 37° C. in 5% CO₂, 95% air and 100% relative humidity. At the end of the incubation, cell viability was quantified with CellTiter-Glo® assay. The drug concentration resulting in growth inhibition of 50% (IC₅₀) was calculated using CDD Vault tool.

To further confirm that compounds were activated by human AKR1C3, H460 proliferation assay were also performed in the presence of 3 uM ASP951, an AKR1C₃ specific inhibitor. ASP9521 were added to the plates 4 hours prior to the addition of compounds.

Selected results according to this example are shown in the Tables below:

| | CTG H460: | CTG H460: |
|---|---|---|
| | Avg IC₅₀ | Avg IC₅₀ |
| | (nM) [Assay | (nM) [Assay |
| Compound | Condition: | Condition: |
| No. | DMSO] | ASP9521] |
| 1 | D | >10.0E+03 |
| 2 | D | >10.0E+03 |
| 3 | D | >3.41E+03 |
| 4 | D | >10.0E+03 |
| 5 | D | >10.0E+03 |
| 6 | D | >10.0E+03 |
| 7 | B | 3950 |
| 8 | A | 1790 |
| 9 | A | 1720 |
| 10 | A | 271 |
| 11 | D | 2050 |
| 12 | A | 89 |
| 13 | A | 195 |
| 14 | A | 553 |
| 15 | A | 947 |
| 16 | B | 261 |
| 17 | B | 758 |
| 18 | A | 501 |
| 19 | A | 593 |
| 20 | A | 1190 |
| 21 | A | 1420 |
| 22 | B | 1640 |
| 23 | A | 75.9 |
| 24 | A | 916 |
| 25 | A | 92.9 |
| 26 | A | 298 |
| 27 | A | 1170 |
| 28 | A | 870 |
| 29 | A | 1220 |
| 30 | A | 1510 |
| 31 | A | 1550 |
| 32 | A | 145 |
| 33 | A | 476 |
| 34 | B | 822 |
| 35 | A | 472 |
| 36 | B | 527 |
| 37 | A | 178 |
| 38 | A | 537 |
| 39 | A | 561 |
| 40 | A | 929 |
| 41 | A | 1090 |
| 42 | A | 123 |
| 43 | A | 314 |
| 44 | D | 1940 |
| 45 | B | 5550 |
| 46 | D | 973 |
| 47 | B | 1050 |
| 48 | D | 1010 |
| 49 | A | 920 |
| 50 | A | 335 |
| 51 | A | 327 |
| 52 | B | 700 |
| 53 | A | 945 |
| 54 | A | 516 |
| 55 | B | 3120 |
| 56 | A | 447 |
| 57 | A | 219 |
| 58 | C | 1010 |
| 59 | A | 1360 |
| 60 | D | 1010 |
| 61 | B | 1010 |
| 62 | D | 6100 |
| 63 | B | 7430 |
| 64 | A | 722 |
| 65 | A | 279 |
| 66 | A | 736 |
| 67 | A | 541 |
| 68 | A | 1160 |
| 69 | A | 573 |
| 70 | A | 380 |
| 71 | A | 31.9 |
| 72 | C | 1390 |
| 73 | A | 943 |
| 74 | D | 1270 |
| 75 | B | 2720 |
| 76 | B | 1120 |
| 77 | A | 1890 |
| 78 | D | 1150 |
| 79 | B | 1610 |
| 80 | A | 909 |
| 81 | A | 829 |
| 82 | A | 181 |
| 83 | A | 1170 |
| 84 | B | 1020 |
| 85 | A | 5280 |
| 86 | B | 1230 |
| 87 | B | 5890 |
| 88 | C | 3570 |
| 89 | B | 6150 |
| 90 | D | 9470 |
| 91 | D | >10.0E+03 |
| 92 | A | 1420 |
| 93 | D | 878 |
| 94 | B | 1450 |
| 95 | C | 1310 |
| 96 | B | 5270 |
| 97 | A | 497 |
| 98 | A | 205 |
| 99 | A | 1010 |
| 100 | A | 618 |
| 101 | A | 1210 |
| 102 | A | 919 |
| 103 | A | 811 |
| 104 | A | 180 |
| 105 | B | 1300 |
| 106 | A | 416 |
| 107 | B | 6500 |
| 108 | A | 1710 |
| 109 | B | 6450 |
| 110 | A | 2420 |
| 111 | A | 1100 |
| 112 | A | 243 |
| 113 | A | 650 |
| 114 | A | 1280 |
| 115 | B | 4730 |
| 116 | B | 5560 |
| 117 | D | 1030 |
| 118 | B | 4510 |
| 119 | A | 644 |
| 120 | A | 2080 |
| 121 | A | 33.8 |
| 122 | B | 3090 |
| 123 | A | 572 |
| 124 | A | 547 |
| 125 | A | 766 |
| 126 | A | 159 |
| 127 | B | 291 |
| 128 | A | 491 |
| 129 | B | 982 |
| 130 | B | 389 |
| 131 | A | 314 |
| 132 | A | 1250 |
| 133 | B | 1620 |
| 134 | B | 1510 |
| 135 | A | 482 |
| 136 | C | 913 |
| 137 | A | 538 |
| 138 | A | 579 |
| 139 | A | 839 |
| 140 | A | 436 |
| 141 | A | 366 |
| 142 | A | 4130 |
| 143 | A | 1720 |
| 144 | A | 347 |
| 145 | A | 1120 |
| 146 | A | 453 |
| 147 | A | 311 |
| 148 | A | 955 |
| 149 | A | 372 |
| 150 | A | 297 |
| 151 | A | 306 |
| 152 | A | 1400 |
| 153 | A | 477 |
| 154 | A | 309 |
| 155 | A | 128 |
| 156 | B | 895 |
| 157 | B | 1240 |
| 158 | C | 4680 |
| 159 | D | >10.0E+03 |
| 160 | A | 270 |
| 161 | A | 339 |
| 162 | A | 969 |
| 163 | A | 1340 |
| 164 | A | 237 |
| 165 | A | 89.7 |
| 166 | B | 976 |
| 167 | B | 3150 |
| 168 | A | 755 |
| 169 | A | 766 |
| 170 | A | 801 |
| 171 | A | 1.10E+03 |
| 172 | B | 4900 |
| 173 | A | 2980 |
| 174 | A | 244 |
| 175 | A | 139 |
| 176 | A | 2370 |
| 177 | A | 1350 |
| 178 | D | 1630 |
| 179 | D | 1100 |
| 180 | A | 1950 |
| 181 | A | 942 |
| 182 | D | >10.0E+03 |
| 183 | B | 1840 |
| 184 | B | 2640 |
| 185 | B | 889 |
| 186 | B | >10.0E+03 |
| 187 | B | >10.0E+03 |
| 188 | A | 2070 |
| 189 | A | 1190 |
| 190 | A | 984 |
| 191 | A | 661 |
| 192 | A | 1090 |
| 193 | A | 880 |
| 194 | B | 1290 |
| 195 | B | 4980 |
| 196 | D | 6370 |
| 197 | D | 1760 |
| 198 | A | 2230 |
| 199 | A | 416 |
| 200 | B | 434 |
| 201 | A | 605 |
| 202 | A | 1390 |
| 203 | A | 959 |
| 204 | B | 1550 |
| 205 | A | 184 |
| 206 | A | 82.3 |
| 207 | A | 217 |
| 208 | A | 184 |
| 209 | A | 517 |
| 210 | A | 294 |
| 211 | A | 1270 |
| 212 | A | 958 |
| 213 | A | 1200 |
| 214 | A | 913 |
| 215 | A | 3780 |
| 216 | A | 3240 |
| 217 | A | 1600 |
| 218 | A | 1370 |
| 219 | A | 2750 |
| 220 | A | 5050 |
| 221 | A | 1210 |
| 222 | B | 3260 |
| 223 | A | 2590 |
| 224 | A | 6460 |
| 225 | C | 9530 |
| 226 | B | >10.0E+03 |
| 227 | B | 9760 |
| 228 | B | >10.0E+03 |
| 229 | A | 515 |
| 230 | A | 846 |
| 231 | A | 278 |
| 232 | B | 4110 |
| 233 | A | 3890 |

| | CTG H460: | CTG H460: |
|---|---|---|
| | Avg IC₅₀ (nM) | Avg IC₅₀ |
| | [Assay | (nM) [Assay |
| Compound | Condition: | Condition: |
| No. | DMSO] | ASP9521] |
| 234 | A | 865 |
| 235 | A | 717 |
| 236 | A | 715 |
| 237 | A | 736 |
| 238 | A | 1160 |
| 239 | A | 1060 |
| 240 | A | 1150 |
| 241 | A | 618 |
| 242 | A | 1270 |
| 243 | A | 919 |
| 244 | A | 834 |
| 245 | A | 416 |
| 246 | A | 522 |
| 247 | A | 764 |
| 248 | A | 1650 |
| 249 | A | 1340 |
| 250 | A | 1220 |
| 251 | A | 1090 |
| 252 | B | 2640 |
| 253 | A | 697 |
| 254 | A | 786 |
| 255 | A | 832 |
| 256 | A | 1240 |
| 257 | A | 1810 |
| 258 | A | 2060 |
| 259 | A | 1550 |
| 260 | A | 734 |
| 261 | B | 5690 |
| 262 | A | 945 |
| 263 | A | 943 |
| 264 | A | 1370 |
| 265 | A | 1300 |
| 266 | B | 716 |
| 267 | A | 639 |
| 268 | A | 1110 |
| 269 | B | 761 |
| 270 | B | 5550 |
| 271 | A | 920 |
| 272 | B | 3120 |
| 273 | A | 1400 |
| 274 | B | 7430 |
| 275 | A | 1350 |
| 276 | A | 1340 |
| 277 | A | 1530 |
| 278 | A | 1560 |
| 279 | A | 1310 |
| 280 | A | 1300 |
| 281 | A | 787 |
| 282 | A | 1240 |
| 283 | A | 1460 |
| 284 | A | 1150 |
| 285 | A | 754 |
| 286 | A | 1870 |
| 287 | A | 1010 |
| 288 | A | 1940 |
| 289 | A | 1090 |
| 290 | A | 274 |
| 291 | A | 1970 |
| 292 | A | 1810 |
| 293 | A | 1050 |
| 294 | A | 1080 |
| 295 | B | 3620 |
| 296 | A | 976 |
| 297 | A | 1300 |
| 298 | A | 1690 |
| 299 | A | 744 |
| 300 | A | 1890 |
| 301 | A | 3730 |
| 302 | B | 6150 |
| 303 | B | 5270 |
| 304 | A | 2130 |
| 305 | A | 572 |
| 306 | A | 1990 |
| 307 | A | 2360 |
| 308 | A | 357 |
| 309 | B | 3070 |
| 310 | A | 2010 |
| 311 | A | 485 |
| 312 | A | 312 |
| 313 | A | 1260 |
| 314 | A | 1590 |
| 315 | A | 679 |
| 316 | A | 781 |
| 317 | A | 1010 |
| 318 | A | 1070 |
| 319 | A | 521 |
| 320 | A | 2230 |
| 321 | B | 6690 |
| 322 | A | 1610 |
| 323 | A | 2690 |
| 324 | A | 445 |
| 325 | A | 608 |
| 326 | A | 1270 |
| 327 | A | 1710 |
| 328 | A | 4230 |
| 329 | A | 2440 |
| 330 | A | 2980 |
| 331 | A | 1050 |
| 332 | A | 1960 |
| 333 | A | 1310 |
| 334 | A | 824 |
| 335 | A | 1910 |
| 336 | A | 1680 |
| 337 | B | 5800 |
| 338 | B | 5560 |
| 339 | A | 1360 |
| 340 | B | 4390 |
| 341 | A | 1920 |
| 342 | B | 4270 |
| 343 | A | 2300 |
| 344 | A | 1510 |
| 345 | A | 2110 |
| 346 | A | 604 |
| 347 | A | 1300 |
| 348 | A | 827 |
| 349 | A | 498 |
| 350 | A | 1870 |
| 351 | A | 1570 |
| 352 | A | 268 |
| 353 | A | 1010 |
| 354 | A | 768 |
| 355 | A | 1130 |
| 356 | A | 1990 |
| 357 | A | 467 |
| 358 | A | 1740 |
| 359 | A | 1470 |
| 360 | A | 391 |
| 361 | B | 3910 |
| 362 | A | 1660 |
| 363 | A | 584 |
| 364 | B | 4030 |
| 365 | A | 1320 |
| 366 | A | 636 |
| 367 | A | 321 |
| 368 | B | 3360 |
| 369 | A | 938 |
| 370 | A | 967 |
| 371 | A | 591 |
| 372 | A | 332 |
| 373 | A | 672 |
| 374 | A | 468 |
| 375 | A | 2520 |
| 376 | A | 1850 |
| 377 | A | 1330 |
| 378 | A | 440 |
| 379 | A | 447 |
| 380 | A | 722 |
| 381 | A | 312 |
| 382 | A | 320 |
| 383 | B | 4240 |
| 384 | A | 1490 |
| 385 | A | 819 |
| 386 | A | 1410 |
| 387 | A | 1860 |
| 388 | A | 1430 |
| 389 | A | 520 |
| 390 | A | 1410 |
| 391 | A | 1810 |
| 392 | A | 785 |
| 393 | A | 1230 |
| 394 | A | 388 |
| 395 | A | 1260 |
| 396 | B | 2480 |
| 397 | A | 956 |
| 398 | A | 1910 |
| 399 | A | 1110 |
| 400 | A | 452 |
| 401 | A | 454 |
| 402 | A | 712 |
| 403 | A | 1460 |
| 404 | A | 1020 |
| 405 | A | 871 |
| 406 | A | 1320 |
| 407 | A | 380 |
| 408 | A | 829 |
| 409 | A | 1170 |
| 410 | B | 4660 |
| 411 | B | 6450 |
| 412 | A | 2420 |
| 413 | A | 1180 |
| 414 | A | 1280 |
| 415 | B | 5560 |
| 416 | A | 275 |
| 417 | A | 712 |
| 418 | A | 1210 |
| 419 | A | 1180 |
| 420 | A | 675 |
| 421 | A | 1400 |
| 422 | A | 821 |
| 423 | A | 524 |
| 424 | A | 779 |
| 425 | A | 5270 |
| 426 | A | 638 |
| 427 | A | 756 |
| 428 | A | 1200 |
| 429 | A | 1960 |
| 430 | A | 4860 |
| 431 | A | 238 |
| 432 | A | 4170 |
| 433 | A | 2590 |
| 434 | A | 1140 |
| 435 | A | 482 |
| 436 | A | 138 |
| 437 | A | 235 |
| 438 | A | 416 |
| 439 | A | 2090 |
| 440 | A | 897 |
| 441 | A | 158 |
| 442 | A | 667 |
| 443 | A | 571 |
| 444 | A | 192 |
| 445 | A | 357 |
| 446 | A | 1210 |
| 447 | A | 547 |
| 448 | A | 647 |
| 449 | A | 570 |
| 450 | A | 1060 |
| 451 | A | 1030 |
| 452 | A | 2240 |
| 453 | A | 819 |
| 454 | A | 559 |
| 455 | A | 1660 |
| 456 | A | 940 |
| 457 | A | 768 |
| 458 | A | 506 |
| 459 | A | 845 |
| 460 | A | 1230 |
| 461 | A | 547 |
| 462 | A | 917 |
| 463 | A | 478 |
| 464 | A | 1390 |
| 465 | A | 705 |
| 466 | A | 975 |
| 467 | A | 4160 |
| 468 | A | 660 |
| 469 | A | 1130 |
| 470 | A | 1150 |
| 471 | A | 3060 |
| 472 | A | 583 |
| 473 | A | 329 |
| 474 | A | 918 |
| 475 | A | 509 |
| 476 | A | 1090 |
| 477 | A | 627 |
| 478 | A | 1690 |
| 479 | A | 1630 |
| 480 | A | 1760 |
| 481 | A | 1120 |
| 482 | A | 1340 |
| 483 | A | 735 |
| 484 | A | 1990 |
| 485 | A | 1150 |
| 486 | A | 967 |
| 487 | A | 1010 |
| 488 | A | 857 |
| 489 | A | 1660 |
| 490 | A | 788 |
| 491 | A | 1940 |
| 492 | A | 778 |
| 493 | A | 341 |
| 494 | A | 160 |
| 495 | A | 635 |
| 496 | A | 783 |
| 497 | A | 1010 |
| 498 | A | 899 |
| 499 | A | 918 |
| 500 | A | 1300 |
| 501 | A | 1080 |
| 502 | A | 546 |
| 503 | A | 1660 |
| 504 | A | 288 |
| 505 | A | 327 |
| 506 | A | 625 |
| 507 | A | 952 |
| 508 | A | 519 |
| 509 | A | 844 |
| 510 | A | 535 |
| 511 | A | 429 |
| 512 | A | 83.3 |
| 513 | A | 682 |
| 514 | A | 993 |
| 515 | B | 7150 |
| 516 | A | 778 |
| 517 | A | 2050 |
| 518 | A | 746 |
| 519 | A | 2370 |
| 520 | A | 1350 |
| 521 | A | 1950 |
| 522 | B | 1840 |
| 523 | A | 2070 |
| 524 | A | 1190 |
| 525 | A | 984 |
| 526 | A | 661 |
| 527 | A | 1090 |
| 528 | A | 880 |
| 529 | A | 2230 |
| 530 | A | 697 |
| 531 | A | 1430 |
| 532 | A | 1230 |
| 533 | B | 7430 |
| 534 | A | 1200 |
| 535 | A | 378 |
| 536 | A | 651 |
| 537 | A | 3710 |
| 538 | A | 711 |
| 539 | A | 2730 |
| 540 | A | 1830 |
| 541 | A | 3260 |
| 542 | A | 963 |
| 543 | A | 2230 |
| 544 | B | 3490 |
| 545 | A | 970 |
| 546 | A | 4690 |
| 547 | A | 1260 |
| 548 | A | 2140 |
| 549 | B | 5130 |
| 550 | A | 2510 |
| 551 | A | 2600 |
| 552 | B | 5870 |
| 553 | B | 2950 |
| 554 | A | 6070 |
| 555 | A | 1490 |
| 556 | A | 1060 |
| 557 | B | 7310 |
| 558 | B | 3960 |
| 559 | B | 7000 |
| 560 | B | 3940 |
| 561 | B | 4230 |
| 562 | A | 3040 |
| 563 | A | 2110 |
| 564 | B | 2690 |
| 565 | B | 5930 |
| 566 | B | 6380 |
| 567 | A | 2110 |
| 568 | A | 1920 |
| 569 | A | 937 |

CTG H460: Avg IC₅₀ (nM) [Assay Condition: DMSO]
A: IC₅₀ < 10 nM;
B: 10 nM ≤ IC₅₀ < 50 nM;
C: 50 nM ≤ IC₅₀ < 100 nM;
D: IC₅₀ ≥ 100 nM

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 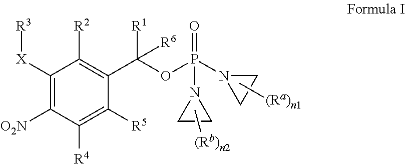
wherein:
(2) R¹ and R², together with the intervening atoms, are joined to form an optionally substituted 4-8 membered carbocyclic or heterocyclic ring, and
R⁴ and R⁵ are as defined in (1); or
(3) R¹ and R⁵, together with the intervening atoms, are joined to form an optionally substituted 4-8 membered carbocyclic or heterocyclic ring; and
R² and R⁴ are as defined in (1); or
(4) R⁴ and R⁵, together with the intervening atoms, are joined to form an optionally substituted 4-8 membered ring; or
R¹ and R² are as defined in (1) or (2); or
(1) R¹ is hydrogen, deuterium, optionally substituted C₁₋₄ alkyl, optionally substituted C₂₋₄ alkenyl, or optionally substituted C₂₋₄ alkynyl; and
R², R⁴, and R⁵ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring; and
wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
R³ is hydrogen, an optionally substituted C₁₋₄ alkyl, or an optionally substituted 3-10 membered ring;
R⁶ is hydrogen, deuterium, optionally substituted C₁₋₄ alkyl, optionally substituted C₂₋₄ alkenyl, or optionally substituted C₂₋₄ alkynyl;
the integers n1 and n2 are each independently 0, 1, 2, 3, or 4;
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atom(s), are joined together to form an optionally substituted 3-6 membered ring, and any remaining instances of R^{a} and/or R^{b} are as defined above.

2. The compound of claim 1, or a pharmaceutically acceptable salt thereof, wherein R¹ and R², together with the intervening atoms, are joined to form an optionally substituted 5-7 membered carbocyclic ring, when substituted, the 5-7 membered carbocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring, and two substituents are optionally joined with the intervening atom(s) to form an optionally substituted 3-6 membered ring.

3. The compound of claim 2, or a pharmaceutically acceptable salt thereof, characterized as having a structure according to Formula I-1, I-2, I-3, I-1-B-E1, or I-1-B-E2: 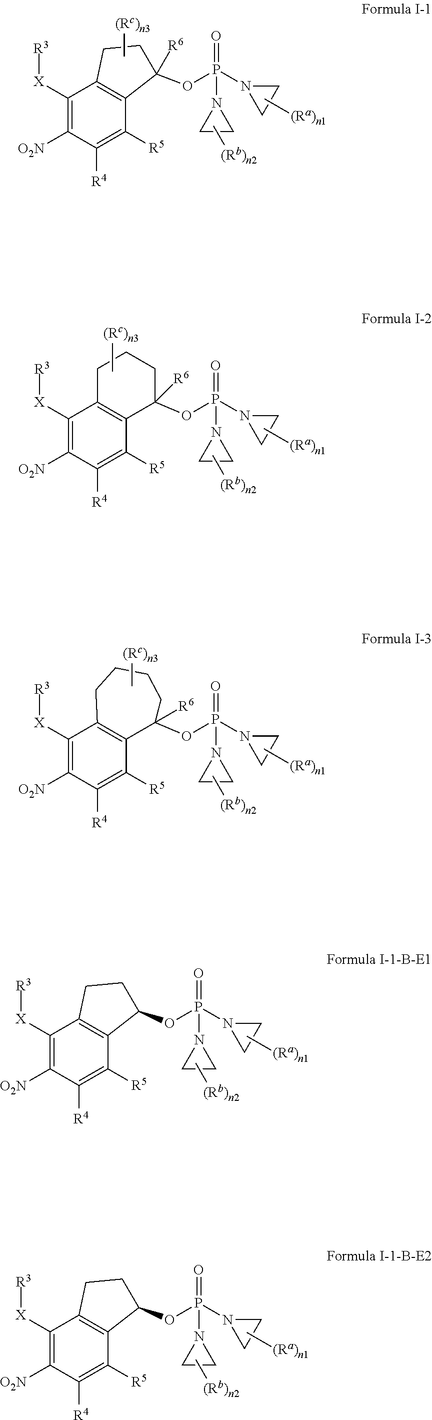
wherein:
the integer n3 is 0, 1, or 2; and
R^{c} at each occurrence is independently oxo, F, OH, NH₂, C₁₋₄ alkyl optionally substituted with F, or C₁₋₄ heteroalkyl having one or two heteroatoms and optionally substituted with F; or two instances of R^{c} are joined with the intervening atom(s) to form an optionally substituted 3-6 membered ring.

4. The compound of claim 3, or a pharmaceutically acceptable salt thereof, wherein n3 is 0.

5. The compound of claim 1, or a pharmaceutically acceptable salt thereof, wherein R¹ and R², together with the intervening atoms, are joined to form an optionally substituted 5-7 membered heterocyclic ring having one or two ring heteroatoms independently selected from O, N, and S, when substituted, the 5-7 membered heterocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring, and two substituents are optionally joined with the intervening atom(s) to form an optionally substituted 3-6 membered ring.

6. The compound of claim 5, or a pharmaceutically acceptable salt thereof, characterized as having a structure according to Formula I-4, I-5, or I-6: 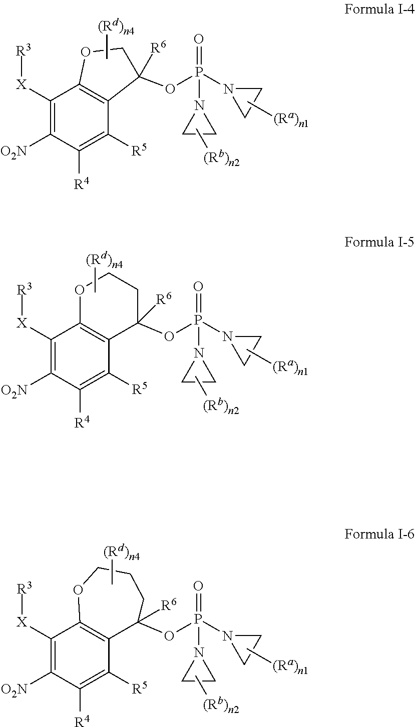
wherein:
the integer n4 is 0, 1, or 2; and
R^{d} at each occurrence is independently oxo, F, OH, NH₂, C₁₋₄ alkyl optionally substituted with F, or C₁₋₄ heteroalkyl having one or two heteroatoms and optionally substituted with F; or two instances of R^{d} are joined with the intervening atom(s) to form an optionally substituted 3-6 membered ring.

7. The compound of claim 6, or a pharmaceutically acceptable salt thereof, wherein n4 is 0.

8. The compound of claim 1, or a pharmaceutically acceptable salt thereof, wherein R¹ and R⁵, together with the intervening atoms, are joined to form an optionally substituted 5-7 membered carbocyclic ring, when substituted, the 5-7 membered carbocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring, and two substituents are optionally joined with the intervening atom(s) to form an optionally substituted 3-6 membered ring.

9. The compound of claim 8, or a pharmaceutically acceptable salt thereof, characterized as having a structure according to Formula I-7, I-8, or I-9: 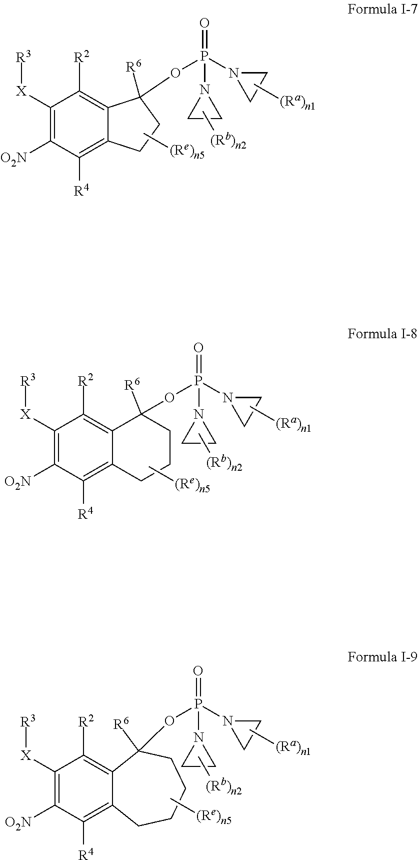
wherein:
the integer n5 is 0, 1, or 2; and
R^{c} at each occurrence is independently oxo, F, OH, NH₂, C₁₋₄ alkyl optionally substituted with F, or C₁₋₄ heteroalkyl having one or two heteroatoms and optionally substituted with F; or two instances of R^{c} are joined with the intervening atom(s) to form an optionally substituted 3-6 membered ring.

10. The compound of claim 9, or a pharmaceutically acceptable salt thereof, wherein n5 is 0.

11. The compound of claim 1, or a pharmaceutically acceptable salt thereof, wherein R¹ and R⁵, together with the intervening atoms, are joined to form an optionally substituted 5-7 membered heterocyclic ring having one or two ring heteroatoms independently selected from O, N, and S, when substituted, the 5-7 membered heterocyclic ring is substituted with 1-3 substituents independently selected from oxo, halogen, OH, NH₂, C₁₋₄ alkyl optionally substituted with F, C₁₋₄ heteroalkyl having one or two heteroatoms and optionally substituted with F, and 3-6 membered ring, and two substituents are optionally joined with the intervening atom(s) to form an optionally substituted 3-6 membered ring.

12. The compound of claim 11, or a pharmaceutically acceptable salt thereof, characterized as having a structure according to Formula I-10, I-11, or I-12: 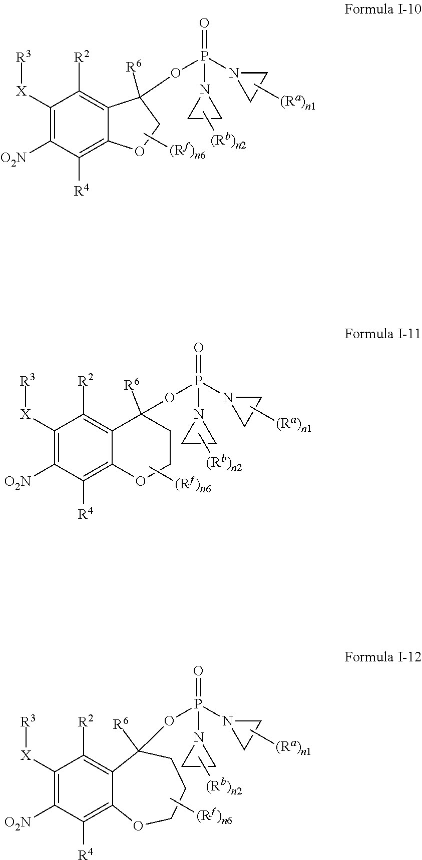
wherein:
the integer n6 is 0, 1, or 2; and
R^{f} at each occurrence is independently oxo, F, OH, NH₂, C₁₋₄ alkyl optionally substituted with F, or C₁₋₄ heteroalkyl having one or two heteroatoms and optionally substituted with F; or two instances of R^{f} are joined with the intervening atom(s) to form an optionally substituted 3-6 membered ring.

13. The compound of claim 12, or a pharmaceutically acceptable salt thereof, wherein n6 is 0.

14. The compound of any one of claims 1-13, or a pharmaceutically acceptable salt thereof, wherein both n1 and n2 are 0.

15. The compound of any one of claims 1-13, or a pharmaceutically acceptable salt thereof, wherein the compound is characterized as having a structure according to Formula I-13:

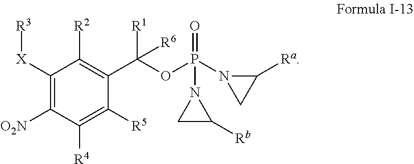

16. The compound of any one of claims 1-15, or a pharmaceutically acceptable salt thereof, wherein R⁶ is hydrogen, deuterium, CH₃, or CF₃.

17. The compound of any one of claims 1-16, or a pharmaceutically acceptable salt thereof, wherein R⁴ is hydrogen.

18. The compound of any one of claims 1-7 and 14-17, or a pharmaceutically acceptable salt thereof, wherein as applicable, R⁵ is hydrogen.

19. The compound of any one of claims 1 and 8-18, or a pharmaceutically acceptable salt thereof, wherein as applicable, R² is hydrogen.

20. The compound of any one of claims 1 and 14-19, or a pharmaceutically acceptable salt thereof, wherein as applicable, R¹ is hydrogen, deuterium, or C₁₋₄ alkyl optionally substituted with F, such as methyl, CF₃, etc.

21. The compound of any one of claims 1-20, or a pharmaceutically acceptable salt thereof, wherein X is O.

22. The compound of any one of claims 1-21, or a pharmaceutically acceptable salt thereof, wherein R³ is an optionally substituted phenyl, an optionally substituted 5 or 6-membered heteroaryl, or an optionally substituted bicyclic heteroaryl (e.g., 8-10 membered bicyclic heteroaryl).

23. The compound of any one of claims 1-21, or a pharmaceutically acceptable salt thereof, wherein R³ is a phenyl which is substituted with 1-3 substituents each independently selected from halogen, CN, OH, NH₂, COOH, CONH₂, G¹, OG¹, SG₁, NHG¹, NG¹G¹, C(O)G¹, COOG¹, CONHG¹, CONG¹G¹, OC(O)G¹, OCOOG¹, OCONHG¹, OCONG¹G¹, NHC(O)G¹, NHCOOG¹, NHCONHG¹, NHCONG¹G¹, NG¹C(O)G¹, NG¹COOG¹, NG¹CONHG¹, NG¹CONG¹G¹, SO₂G¹, SO₂NHG¹, or SO₂NG¹G¹, wherein G¹ at each occurrence is independently an optionally substituted C₁₋₄ alkyl, an optionally substituted C₂₋₄ alkenyl, an optionally substituted C₂₋₄ alkynyl, or an optionally substituted 3-6 membered ring structure, such as cyclopropyl, cyclobutyl, phenyl, pyridyl, etc., or two G¹ of an NG¹G¹ can join together with the nitrogen atom to form an optionally substituted 4-8 membered heterocyclic ring.

24. The compound of any one of claims 1-21, or a pharmaceutically acceptable salt thereof, wherein R³ is a phenyl which is substituted with 1-3 substituents each independently selected from F, Cl, CN, OH, NH₂, COOH, CONH₂, G², OG², NHG², NG²G², C(O)G², COOG², CONHG², CONG²G², SO₂G², SO₂NHG², or SO₂NG²G², wherein G² at each occurrence is independently a C₁₋₄ alkyl, C₃₋₆ cycloalkyl, 3-6 membered heterocyclic ring having 1-2 ring heteroatoms, phenyl, or 5 or 6-membered heteroaryl, each of which is optionally substituted with 1-3 substituents independently selected from F, C₁, OH, NH₂, C₁₋₄ alkyl optionally substituted with 1-3 F, or C₁₋₄ heteroalkyl having 1 or 2 heteroatoms and optionally substituted with 1-3 F, or two G² of an NG²G² can join together with the nitrogen atom to form an optionally substituted 4-8 membered heterocyclic ring having 0 or 1 additional ring heteroatom.

25. The compound of any one of claims 1-21, or a pharmaceutically acceptable salt thereof, wherein R³ is: 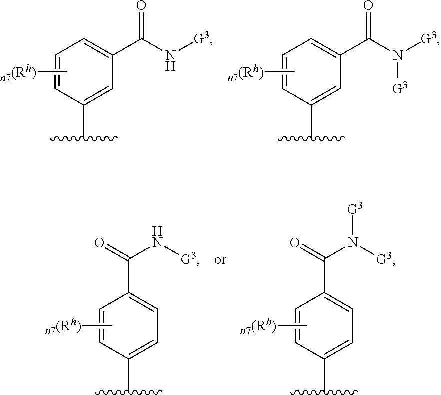
wherein:
G³ at each occurrence is independently an optionally substituted C₁₋₄ alkyl or optionally substituted 3-6 membered ring, such as cyclopropyl, cyclobutyl, oxetanyl (e.g., 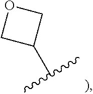 [SMILES: *C1COC1]
etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-8 membered heterocyclic ring having 0 or 1 additional ring heteroatom, wherein the 4-8 membered heterocyclic ring is optionally substituted with 1-3 substituents each independently oxo, F, C₁₋₄ alkyl, OH, NH₂, or C₁₋₄ heteroalkyl having 1-2 heteroatoms;
and wherein:
the integer n7 is 0, 1, or 2; and
R^{h} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

26. The compound of any one of claims 1-21, or a pharmaceutically acceptable salt thereof, characterized as having a structure according to Formula IV-1, IV-7, or IV-8: 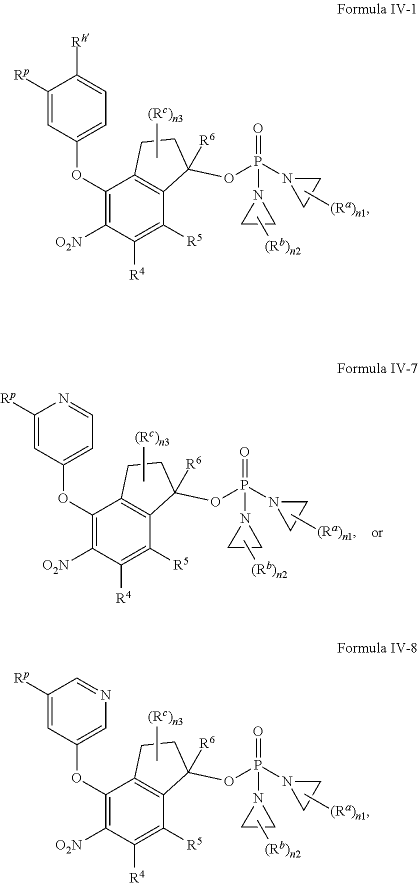
wherein:
R^{P} is halogen, CN, OH, NH₂, COOH, CONH₂, G³, OG³, SG₃, NHG³, NG³G³, C(O)G³, COOG³, CONHG³, CONG³G³, OC(O)G³, OCOOG³, OCONHG³, OCONG³G³, NHC(O)G³, NHCOOG³, NHCONHG³, NHCONG³G³, NG³C(O)G³, NG³COOG³, NG³CONHG³, NG³CONG³G³, SO₂G³, SO₂NHG³, or SO₂NG³G³,
G³ at each occurrence is independently an optionally substituted C₁₋₄ alkyl or optionally substituted 3-6 membered ring, such as cyclopropyl, cyclobutyl, oxetanyl (e.g., 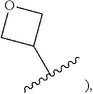 [SMILES: *C1COC1]
etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-8 membered heterocyclic ring having 0 or 1 additional ring heteroatom, wherein the 4-8 membered heterocyclic ring is optionally substituted with 1-3 substituents each independently oxo, F, C₁₋₄ alkyl, OH, NH₂, or C₁₋₄ heteroalkyl having 1-2 heteroatoms;
R^{h′} is hydrogen or R^{h}, and
R^{h} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

27. The compound of claim 25 or 26, or a pharmaceutically acceptable salt thereof, wherein each G³ is independently methyl, 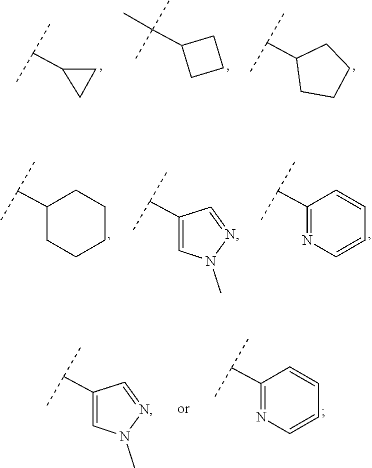
or two G³ together with the nitrogen atom they are both attached to are joined to form

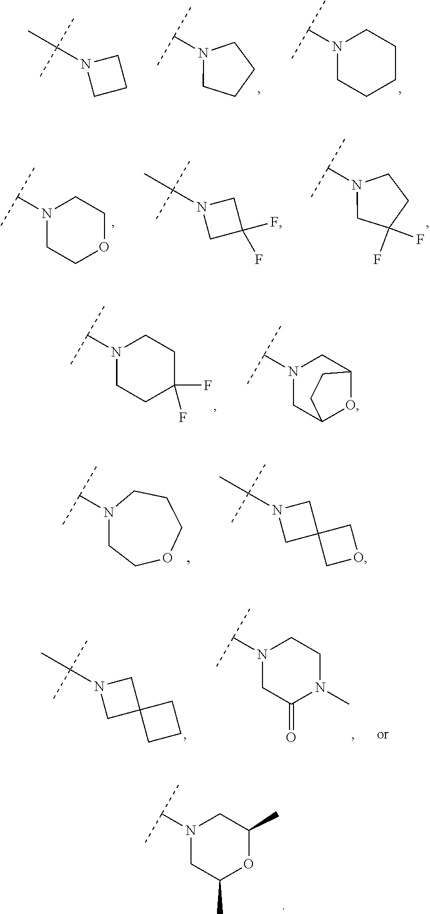

28. The compound of any one of claims 25-27, or a pharmaceutically acceptable salt thereof, wherein n7 is 0, or R^{h′} is hydrogen.

29. The compound of any one of claims 25-27, or a pharmaceutically acceptable salt thereof, wherein n7 is 1, and R^{h} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

30. The compound of any one of claims 1-21, or a pharmaceutically acceptable salt thereof, wherein R³ is: 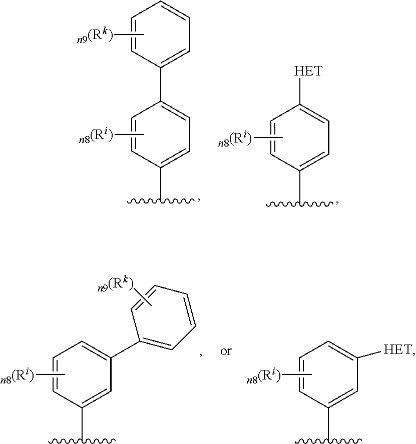
wherein:
the integers n8 and n9 are independently 0, 1, or 2;
HET is a 5 or 6 membered heteroaryl optionally substituted with 1-2 R^{j}; and
each of Rⁱ, R^{j}, and R^{k} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F or OH, or C₁₋₄ heteroalkyl having 1-2 heteroatoms and optionally substituted with F.

31. The compound of claim 30, or a pharmaceutically acceptable salt thereof, characterized as having a structure according to Formula IV-2, IV-3, IV-4, or IV-5: 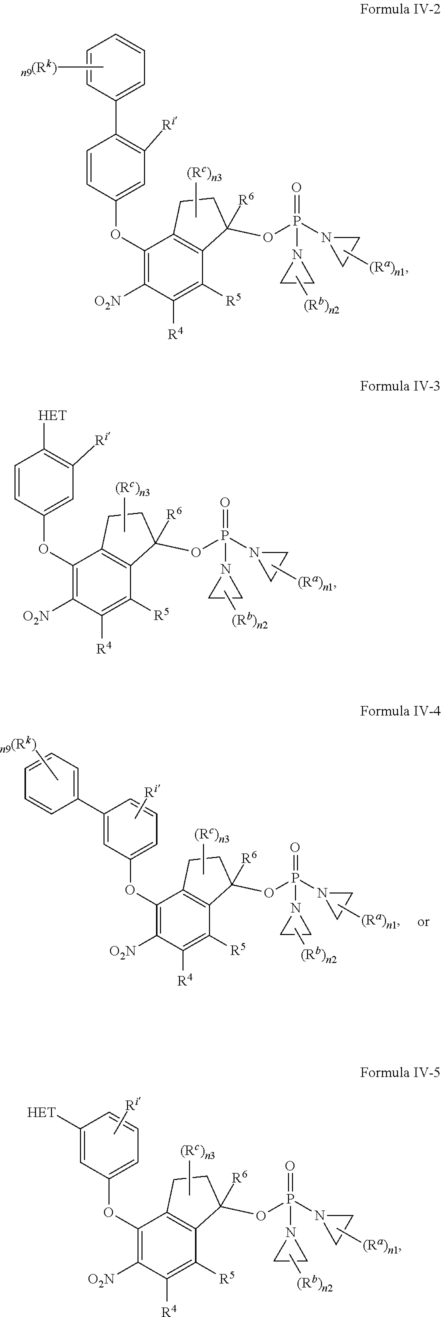
wherein R^{i′} is hydrogen or Rⁱ.

32. The compound of claim 30 or 31, or a pharmaceutically acceptable salt thereof, wherein n8 is 0, or R^{i′} is hydrogen.

33. The compound of claim 30 or 31, or a pharmaceutically acceptable salt thereof, wherein n8 is 1 and Rⁱ is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

34. The compound of any one of claims 30-33, or a pharmaceutically acceptable salt thereof, wherein n9 is 0.

35. The compound of any one of claims 30-33, or a pharmaceutically acceptable salt thereof, wherein n9 is 1 and R^{k} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

36. The compound of any one of claims 30-35, or a pharmaceutically acceptable salt thereof, wherein HET is (1) a pyridyl 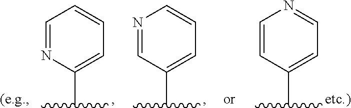
pyrimidinyl 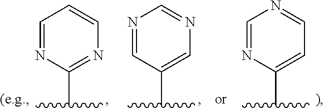
pyrazinyl 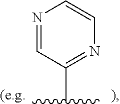 [SMILES: *c1cnccn1]
pyridinonyl 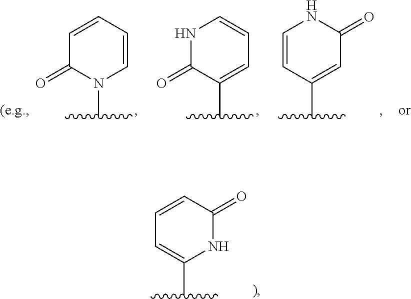
or pyrimidinonyl 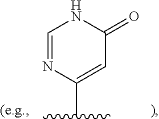 [SMILES: *c1cc(=O)[nH]cn1]
optionally substituted with 1-2 R^{j}, wherein R^{j} at each occurrence is independently F, OH, C₁₋₄ alkyl optionally substituted with F, or C₁₋₄ heteroalkyl having 1-2 heteroatoms and optionally substituted with F; or (2) a pyrazole, oxadiazole, thiadiazole, triazole, tetrazole, thiazole, oxazole, or imidazole 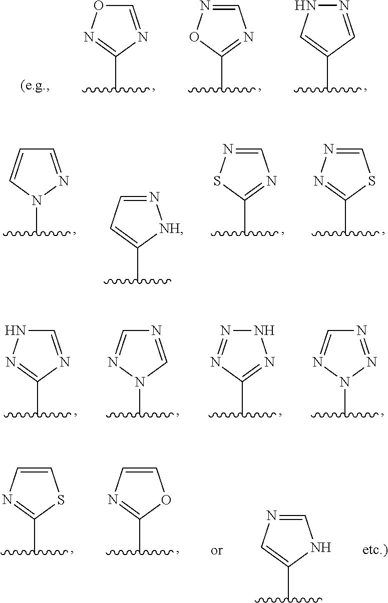
optionally substituted with 1-2 R^{j}, wherein R^{j} at each occurrence is independently F, OH, C₁₋₄ alkyl optionally substituted with F, C₁₋₄ heteroalkyl having 1-2 heteroatoms and optionally substituted with F or 3-4 membered ring such as cyclopropyl or cyclobutyl.

37. The compound of any one of claims 1-21, or a pharmaceutically acceptable salt thereof, wherein R³ is: 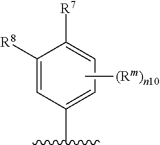
wherein:
the integer n10 is 0, 1, or 2; and
R⁷ and R⁸, together with the intervening atoms, are joined to form a 4-8 membered ring, which is optionally substituted with 1-3 Rⁿ; and
R^{m} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F; and
Rⁿ at each occurrence is independently oxo (as valency permits), halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

38. The compound of claim 37, or a pharmaceutically acceptable salt thereof, characterized as having a structure according to Formula IV-6: 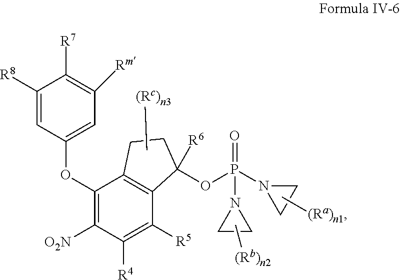
wherein R^{m′} is hydrogen or R^{m}.

39. The compound of claim 37 or 38, or a pharmaceutically acceptable salt thereof, wherein R⁷ and R⁸, together with the intervening atoms, are joined to form a 5 or 6 membered heteroaryl ring having 1-3 ring heteroatoms, which is optionally substituted with 1-2 Rⁿ, wherein Rⁿ is as defined in claim 37.

40. The compound of claim 37 or 38, or a pharmaceutically acceptable salt thereof, wherein R⁷ and R⁸, together with the intervening atoms, are joined to form a 4-7 membered heterocyclyl ring having 1 or 2 ring heteroatoms, which is optionally substituted with 1-2 Rⁿ, wherein Rⁿ is as defined in claim 37.

41. The compound of claim 37 or 38, or a pharmaceutically acceptable salt thereof, wherein R³ is 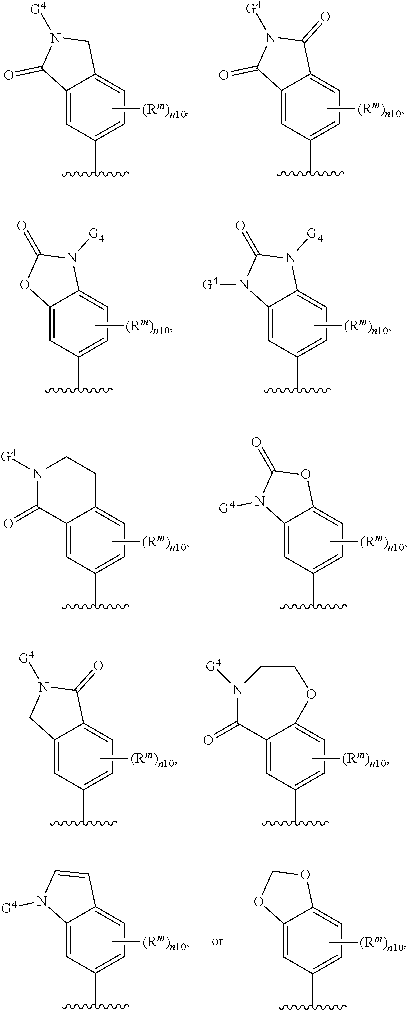
wherein:
G⁴ at each occurrence is independently an optionally substituted C₁₋₄ alkyl, or optionally substituted 3-6 membered ring, such as cyclopropyl, when substituted, the C₁₋₄ alkyl or 3-6 membered ring is substituted with 1-3 substituents each independently F, OH, C₁₋₄ alkyl optionally substituted with F, or C₁₋₄ heteroalkyl having 1-2 heteroatoms and optionally substituted with F.

42. The compound of any one of claims 37-41, or a pharmaceutically acceptable salt thereof, wherein n10 is 0, or R^{m′} is hydrogen.

43. The compound of any one of claims 37-41, or a pharmaceutically acceptable salt thereof, wherein n10 is 1, and R^{m} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

44. A compound of Formula II, or a pharmaceutically acceptable salt thereof: 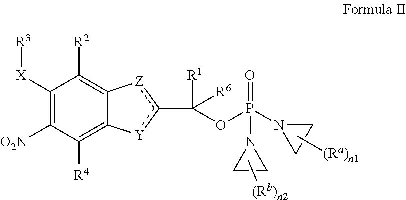
wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
Y and Z are each independently O, S, NR¹¹ or CR¹², as valency permits, provided that the 5-membered ring containing Y and Z is aromatic; wherein:
(i) R¹¹ is hydrogen, an optionally substituted C₁₋₄ alkyl, an optionally substituted 3-6 membered ring, or a nitrogen protecting group, R¹² is hydrogen, halogen, CN, an optionally substituted C₁₋₄ alkyl, an optionally substituted C₁₋₄ heteroalkyl, or an optionally substituted 3-6 membered ring; or
(ii) R¹¹ or R¹², as applicable, together with R¹ and the intervening atoms are joined together to form an optionally substituted 5-8 membered ring;
R¹ is defined in (ii) or is hydrogen, deuterium, optionally substituted C₁₋₄ alkyl, optionally substituted C₂₋₄ alkenyl, or optionally substituted C₂₋₄ alkynyl;
R² and R⁴ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring;
R³ is hydrogen, an optionally substituted C₁₋₄ alkyl, or an optionally substituted 3-10 membered ring;
R⁶ is hydrogen, deuterium, optionally substituted C₁₋₄ alkyl, optionally substituted C₂₋₄ alkenyl, or optionally substituted C₂₋₄ alkynyl;
the integers n1 and n2 are each independently 0, 1, 2, 3, or 4; and
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atoms, are joined together to form an optionally substituted 3-6 membered ring, and any remaining instances of R^{a} and/or R^{b} are as defined above.

45. The compound of claim 44, or a pharmaceutically acceptable salt thereof, characterized as having a structure according to Formula II-1:

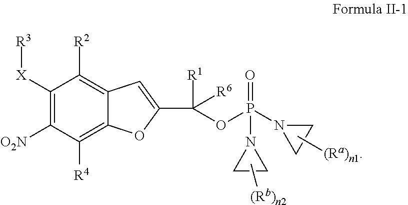

46. The compound of claim 44 or 45, or a pharmaceutically acceptable salt thereof, wherein both n1 and n2 are 0.

47. The compound of claim 44 or 45, or a pharmaceutically acceptable salt thereof, wherein the compound is characterized as having a structure according to Formula II-2:

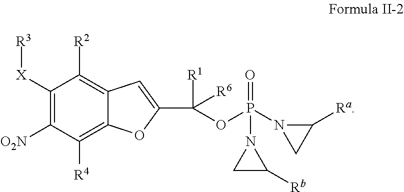

48. The compound of any one of claims 44-47, or a pharmaceutically acceptable salt thereof, wherein R⁶ is hydrogen, deuterium, CH₃, or CF₃.

49. The compound of any one of claims 44-48, or a pharmaceutically acceptable salt thereof, wherein R⁴ is hydrogen.

50. The compound of any one of claims 44-49, or a pharmaceutically acceptable salt thereof, wherein as applicable, R² is hydrogen.

51. The compound of any one of claims 44-50, or a pharmaceutically acceptable salt thereof, wherein as applicable, R¹ is hydrogen, deuterium, or C₁₋₄ alkyl optionally substituted with F, such as methyl, CF₃, etc.

52. The compound of any one of claims 44-51, or a pharmaceutically acceptable salt thereof, wherein X is O.

53. The compound of any one of claims 44-52, or a pharmaceutically acceptable salt thereof, wherein R³ is an optionally substituted phenyl, an optionally substituted 5 or 6-membered heteroaryl, or an optionally substituted bicyclic heteroaryl (e.g., 8-10 membered bicyclic heteroaryl).

54. The compound of any one of claims 44-52, or a pharmaceutically acceptable salt thereof, wherein R³ is a phenyl which is substituted with 1-3 substituents each independently selected from halogen, CN, OH, NH₂, COOH, CONH₂, G¹, OG¹, SG₁, NHG¹, NG¹G¹, C(O)G¹, COOG¹, CONHG¹, CONG¹G¹, OC(O)G¹, OCOOG¹, OCONHG¹, OCONG¹G¹, NHC(O)G¹, NHCOOG¹, NHCONHG¹, NHCONG¹G¹, NG¹C(O)G¹, NG¹COOG¹, NG¹CONHG¹, NG¹CONG¹G¹, SO₂G¹, SO₂NHG¹, or SO₂NG¹G¹, wherein G¹ at each occurrence is independently an optionally substituted C₁₋₄ alkyl, an optionally substituted C₂₋₄ alkenyl, an optionally substituted C₂₋₄ alkynyl, or an optionally substituted 3-6 membered ring structure, such as cyclopropyl, cyclobutyl, phenyl, pyridyl, etc., or two G¹ of an NG¹G¹ can join together with the nitrogen atom to form an optionally substituted 4-8 membered heterocyclic ring.

55. The compound of any one of claims 44-52, or a pharmaceutically acceptable salt thereof, wherein R³ is a phenyl which is substituted with 1-3 substituents each independently selected from F, Cl, CN, OH, NH₂, COOH, CONH₂, G², OG², NHG², NG²G², C(O)G², COOG², CONHG², CONG²G², SO₂G², SO₂NHG², or SO₂NG²G², wherein G² at each occurrence is independently a C₁₋₄ alkyl, C₃₋₆ cycloalkyl, 3-6 membered heterocyclic ring having 1-2 ring heteroatoms, phenyl, or 5 or 6-membered heteroaryl, each of which is optionally substituted with 1-3 substituents independently selected from F, Cl, OH, NH₂, C₁₋₄ alkyl optionally substituted with 1-3 F, or C₁₋₄ heteroalkyl having 1 or 2 heteroatoms and optionally substituted with 1-3 F, or two G² of an NG²G² can join together with the nitrogen atom to form an optionally substituted 4-8 membered heterocyclic ring having 0 or 1 additional ring heteroatoms.

56. The compound of any one of claims 44-52, or a pharmaceutically acceptable salt thereof, wherein R³ is: 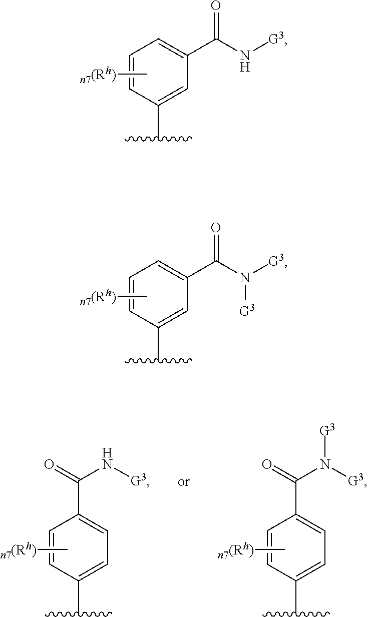
wherein:
G³ at each occurrence is independently an optionally substituted C₁₋₄ alkyl or optionally substituted 3-6 membered ring, such as cyclopropyl, cyclobutyl, oxetanyl 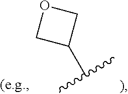 [SMILES: *C1COC1]
etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-8 membered heterocyclic ring having 0 or 1 additional ring heteroatom, wherein the 4-8 membered heterocyclic ring is optionally substituted with 1-3 substituents each independently oxo, F, C₁₋₄ alkyl, OH, NH₂, or C₁₋₄ heteroalkyl having 1-2 heteroatoms;
and wherein:
the integer n7 is 0, 1, or 2; and
R^{h} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

57. The compound of any one of claims 44-52, or a pharmaceutically acceptable salt thereof, characterized as having a structure according to Formula V-1: 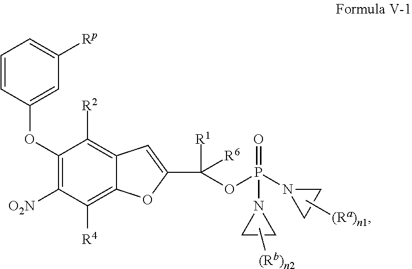
wherein:
R^{P} is halogen, CN, OH, NH₂, COOH, CONH₂, G³, OG³, SG₃, NHG³, NG³G³, C(O)G³, COOG³, CONHG³, CONG³G³, OC(O)G³, OCOOG³, OCONHG³, OCONG³G³, NHC(O)G³, NHCOOG³, NHCONHG³, NHCONG³G³, NG³C(O)G³, NG³COOG³, NG³CONHG³, NG³CONG³G³, SO₂G³, SO₂NHG³, or SO₂NG³G³,
G³ at each occurrence is independently an optionally substituted C₁₋₄ alkyl or optionally substituted 3-6 membered ring, such as cyclopropyl, cyclobutyl, oxetanyl 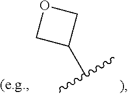 [SMILES: *C1COC1]
etc., or a deuterated analog of the C₁₋₄ alkyl or 3-6 membered ring, when substituted, the C₁₋₄ alkyl or 3-6 membered ring is substituted with 1-3 substituents each independently F, OH, C₁₋₄ alkyl optionally substituted with F, C₁₋₄ heteroalkyl having 1-2 heteroatoms and optionally substituted with F, or 3-6 membered ring (e.g., cyclopropyl, cyclobutyl, or oxetanyl) optionally substituted with F; or
two G³ together with the nitrogen atom they are both attached to are joined to form a 4-7 membered heterocyclic ring having 0 or 1 additional ring heteroatom, wherein the 4-7 membered heterocyclic ring is optionally substituted with 1-3 substituents each independently oxo, F, C₁₋₄ alkyl, OH, NH₂, or C₁₋₄ heteroalkyl having 1-2 heteroatoms.

58. The compound of claim 56 or 57, or a pharmaceutically acceptable salt thereof, wherein G³ is independently methyl, 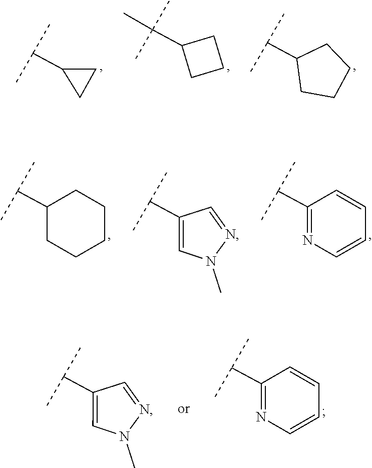
or two G³ together with the nitrogen atom they are both attached to are joined to form

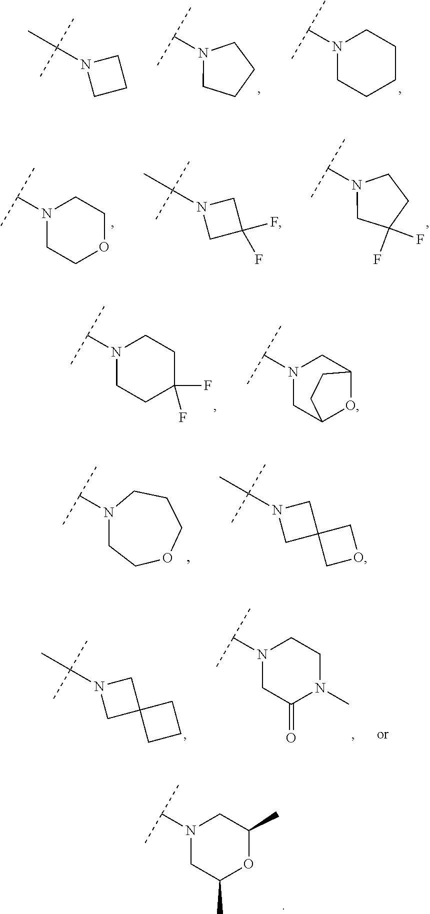

59. The compound of claim 56 or 58, or a pharmaceutically acceptable salt thereof, wherein n7 is 0.

60. The compound of claim 56 or 58, or a pharmaceutically acceptable salt thereof, wherein n7 is 1, and R^{h} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

61. The compound of any one of claims 44-52, or a pharmaceutically acceptable salt thereof, wherein R³ is: 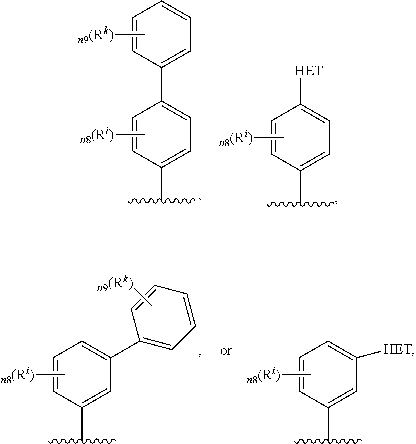
wherein:
the integers n8 and n9 are independently 0, 1, or 2;
HET is a 5 or 6 membered heteroaryl optionally substituted with 1-2 R^{j}; and
each of Rⁱ, R^{j}, and R^{k} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

62. The compound of claim 61, or a pharmaceutically acceptable salt thereof, wherein n8 is 0.

63. The compound of claim 61, or a pharmaceutically acceptable salt thereof, wherein n8 is 1 and Rⁱ is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

64. The compound of any one of claims 61-63, or a pharmaceutically acceptable salt thereof, wherein n9 is 0.

65. The compound of any one of claims 61-63, or a pharmaceutically acceptable salt thereof, wherein n9 is 1 and R^{k} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

66. The compound of any one of claims 61-65, or a pharmaceutically acceptable salt thereof, wherein HET is (1) a pyridyl 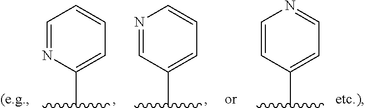
pyrimidinyl 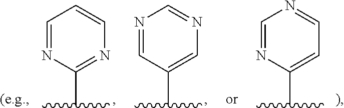
pyrazinyl 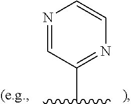 [SMILES: *c1cnccn1]
pyridinonyl 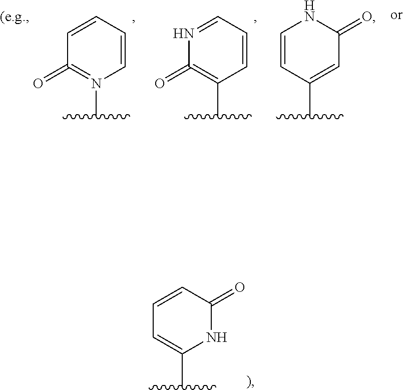
or pyrimidinonyl 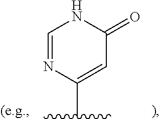 [SMILES: *c1cc(=O)[nH]cn1]
optionally substituted with 1-2 R^{j}, wherein R^{j} at each occurrence is independently F, OH, C₁₋₄ alkyl optionally substituted with F, or C₁₋₄ heteroalkyl having 1-2 heteroatoms and optionally substituted with F; or (2) a pyrazole, oxadiazole, thiadiazole, triazole, tetrazole, thiazole, oxazole, or imidazole 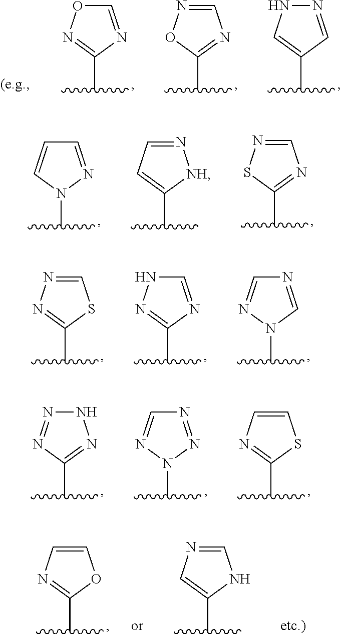
optionally substituted with 1-2 Rⁱ, wherein R^{j} at each occurrence is independently F, OH, C₁₋₄ alkyl optionally substituted with F, C₁₋₄ heteroalkyl having 1-2 heteroatoms and optionally substituted with F or 3-4 membered ring such as cyclopropyl or cyclobutyl.

67. The compound of any one of claims 44-52, or a pharmaceutically acceptable salt thereof, wherein R³ is: 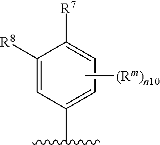
wherein:
the integer n10 is 0, 1, or 2; and
R⁷ and R⁸, together with the intervening atoms, are joined to form a 4-8 membered ring, which is optionally substituted with 1-3 Rⁿ; and
R^{m} at each occurrence is independently a halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F; and
Rⁿ at each occurrence is independently oxo (as valency permits), halogen (e.g., F), CN, OH, C₁₋₄ alkyl, C₁₋₄ alkoxy, or a 3-6 membered ring, wherein the C₁₋₄ alkyl, C₁₋₄ alkoxy, or 3-6 membered ring is optionally substituted with 1-3 substituents each independently F, OH, C₁₋₄ alkyl optionally substituted with F, or C₁₋₄ heteroalkyl having 1-2 heteroatoms and optionally substituted with F.

68. The compound of claim 67, or a pharmaceutically acceptable salt thereof, wherein R⁷ and R⁸, together with the intervening atoms, are joined to form a 5 or 6 membered heteroaryl ring having 1-3 ring heteroatoms, which is optionally substituted with 1-2 Rⁿ, wherein Rⁿ is as defined in claim 67.

69. The compound of claim 67, or a pharmaceutically acceptable salt thereof, wherein R⁷ and R⁸, together with the intervening atoms, are joined to form a 4-7 membered heterocyclyl ring having 1 or 2 ring heteroatoms, which is optionally substituted with 1-2 Rⁿ, wherein Rⁿ is as defined in claim 67.

70. The compound of claim 67, or a pharmaceutically acceptable salt thereof, wherein R³ is 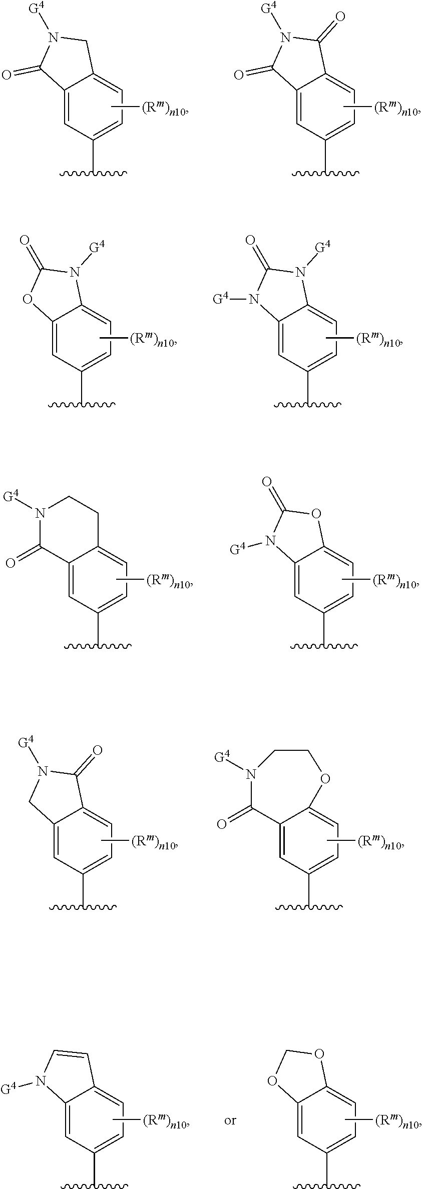
wherein:
G⁴ at each occurrence is independently an optionally substituted C₁₋₄ alkyl, or optionally substituted 3-6 membered ring, such as cyclopropyl, when substituted, the C₁₋₄ alkyl or 3-6 membered ring is substituted with 1-3 substituents each independently F, OH, C₁₋₄ alkyl optionally substituted with F, or C₁₋₄ heteroalkyl having 1-2 heteroatoms and optionally substituted with F.

71. The compound of any one of claims 67-70, or a pharmaceutically acceptable salt thereof, wherein n10 is 0.

72. The compound of any one of claims 67-70, or a pharmaceutically acceptable salt thereof, wherein n10 is 1, and R^{m} is a halogen (e.g., F), CN, C₁₋₄ alkyl optionally substituted with F, C₁₋₄ alkoxy optionally substituted with F, or a 3-6 membered ring optionally substituted with 1-2 substituents each independently F, OH, C₁₋₄ alkyl optionally substituted with F, or C₁₋₄ heteroalkyl having 1-2 heteroatoms and optionally substituted with F.

73. A compound of Formula III, or a pharmaceutically acceptable salt thereof: 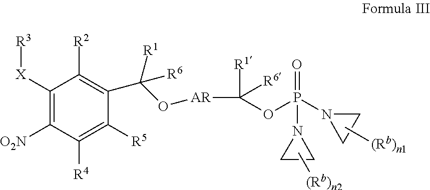
(1) R¹ is hydrogen, deuterium, optionally substituted C₁₋₄ alkyl, optionally substituted C₂₋₄ alkenyl, or optionally substituted C₂₋₄ alkynyl; and
R², R⁴, and R⁵ are each independently hydrogen, halogen (e.g., F), optionally substituted C₁₋₄ alkyl, optionally substituted C₂₋₄ alkenyl, optionally substituted C₂₋₄ alkynyl, optionally substituted C₁₋₄ alkoxy, or an optionally substituted 3-5 membered ring; or
(2) R¹ and R², together with the intervening atoms, are joined to form an optionally substituted 4-8 membered carbocyclic or heterocyclic ring, and
R⁴ and R⁵ are as defined in (1); or
(3) R¹ and R⁵, together with the intervening atoms, are joined to form an optionally substituted 4-8 membered carbocyclic or heterocyclic ring; and
R² and R⁴ are as defined in (1); or
(4) R⁴ and R⁵, together with the intervening atoms, are joined to form an optionally substituted 4-8 membered ring; and
R¹ and R² are as defined in (1) or (2); and
wherein:
X is O, S, NR¹⁰, an optionally substituted C₁₋₄ alkylene, or an optionally substituted C₁₋₄ heteroalkylene, wherein R¹⁰ is hydrogen, an optionally substituted C₁₋₄ alkyl, an optionally substituted 3-6 membered ring, or a nitrogen protecting group;
AR represents an optionally substituted arylene or optionally substituted heteroarylene;
R³ is hydrogen, an optionally substituted C₁₋₄ alkyl, or an optionally substituted 3-10 membered ring;
R^{l′} is hydrogen, deuterium, optionally substituted C₁₋₄ alkyl, optionally substituted C₂₋₄ alkenyl, or optionally substituted C₂₋₄ alkynyl;
R⁶ and R^{6′} are each independently hydrogen, deuterium, optionally substituted C₁₋₄ alkyl, optionally substituted C₂₋₄ alkenyl, or optionally substituted C₂₋₄ alkynyl;
the integers n1 and n2 are each independently 0, 1, 2, 3, or 4; and
each of R^{a} and R^{b} at each occurrence is independently an optionally substituted C₁₋₄ alkyl or an optionally substituted C₁₋₄ heteroalkylene; or two instances of R^{a} or two instances of R^{b}, together with the intervening atoms, are joined together to form an optionally substituted 3-6 membered ring, and any remaining instances of R^{a} and/or R^{b} are as defined above.

74. The compound of any one of claims 1-73, wherein the carbon connected to R⁶ has S-configuration.

75. The compound of any one of claims 1-73, wherein the carbon connected to R⁶ has R-configuration.

76. A compound selected from Examples 1-569 or the compounds shown in Tables A1-A18 herein, a stereoisomer thereof, a deuterated analog thereof, or a pharmaceutically acceptable salt thereof.

77. A pharmaceutical composition comprising the compound of any one of claims 1-76, or a pharmaceutically acceptable salt thereof, and a pharmaceutically acceptable excipient.

78. A method of treating cancer in a subject in need thereof, the method comprising administering to the subject a therapeutically effective amount of the compound of any one of claims 1-76, or a pharmaceutically acceptable salt thereof, or the pharmaceutical composition of claim 77.

79. The method of claim 78, wherein the cancer is selected from the group consisting of a cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid; and acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyo sarcoma, renal cell tumor, topical skin lesion, veticulum cell sarcoma, and Wilm's tumor.

80. The method of claim 78, wherein the cancer is liver cancer, non-small cell lung cancer, melanoma, renal cell carcinoma, or prostate cancer.

81. The method of any one of claims 78-80, wherein the cancer is characterized as having abnormal AKR1C₃ activity and/or overexpression of AKR1C₃.
